(19) United States
(12) Patent Application Publication
(10) Pub. No.: US 2024/0344062 A1
(43) Pub. Date: Oct. 17, 2024

(54) RIBOSOMAL RNA (rRNA) VARIANTS POSSESSING ENHANCED PROTEIN PRODUCTION CAPABILITIES

(71) Applicant: THE BROAD INSTITUTE, INC., Cambridge, MA (US)

(72) Inventors: Ahmed H. Badran, Somerville, MA (US); Fan Liu, Cambridge, MA (US); Sinisa Bratulic, Cambridge, MA (US)

(73) Assignee: THE BROAD INSTITUTE, INC., Cambridge, MA (US)

(21) Appl. No.: 18/682,754

(22) PCT Filed: Aug. 10, 2022
(86) PCT No.: PCT/US2022/074736

§ 371 Date: Feb. 9, 2024

Related U.S. Application Data
(60) Provisional application No. 63/232,155, filed on Aug. 11, 2021.

(51) Int. Cl. C12N 15/11; C12N 15/10; C12N 15/67; C12N 15/70

(52) U.S. Cl. CPC C12N 15/11; C12N 15/1058; C12N 15/67; C12N 15/70

(57) ABSTRACT

The present disclosure relates to compositions, methods and kits for enhancing ribosomal activities in a host cell, especially improvement of the translation activity of heterologous ribosomes within a host cell. Specifically, the instant disclosure provides a number of evolved rRNA sequences, which were remarkably identified to possess enhanced translation activities, improved orthogonal-ribosome binding site (o-RBS) and orthogonal anti-ribosome binding site (o-antiRBS) sequences, host cells possessing deletion or disruption of ribosome hibernation promoting factor (HPF) that thereby exhibit enhanced propagation of selection phage constructs during (PACE), among other aspects. New transgenic organisms harboring heterologous ribosomes and operons are also provided.

### CROSS-REFERENCE TO RELATED APPLICATION

This application is an International Patent Application which claims the benefit of priority under 35 U.S.C. § 119 (e) to U.S. Provisional Application No. 63/232,155, filed on Aug. 11, 2022, entitled, “Ribosomal RNA (rRNA) Variants Possessing Enhanced Protein Production Capabilities.” The entire contents of this patent application are hereby incorporated by reference herein.

### STATEMENT REGARDING FEDERALLY SPONSORED RESEARCH

This invention was made with government support under Grant No. OD024590 awarded by the National Institutes of Health and under Grant No. NNH17ZDA00IN-EXO awarded by the National Aeronautics and Space Administration. The government has certain rights in the invention.

### FIELD OF THE INVENTION

The invention relates generally to compositions, methods and kits for enhancing ribosomal activities in host cells.

### SEQUENCE LISTING

The instant application contains a Sequence Listing which has been filed electronically in eXtensible Markup Language (XML) format and is hereby incorporated by reference in its entirety. Said XML copy, created on Aug. 4, 2022, is named BN00007_1441_SL.xml and is 406 KB in size. A tabulated description of sequences presented in the instant Sequence Listing is provided in Table 1 herein.

### BACKGROUND OF THE INVENTION

Directed modulation of the extraordinary catalytic capabilities of the ribosome represent a promising avenue for synthetic biology, enhancing industrial peptide production, and for identification of new, ribosome-targeting agents/therapeutics, yet the complexity and essentiality of the ribosome have previously hindered significant engineering efforts. Despite these limitations, the existence of extensive sequence identity among ribosomal RNAs (rRNAs) from closely related species has enabled limited heterologous rRNA evaluation in engineered E. coli strains via complementation of a genomic ribosome deficiency. As the ability to perform rRNA complementation has improved, the prospect of optimizing orthogonal rRNA (o-rRNA) properties can now be contemplated. However, a need exists for methods for directing optimization of rRNAs, and for rRNA compositions identified to possess improved qualities (e.g., enhanced translation rate and/or other activities) in host cells, for synthetic biology/evolution and ribosome-targeting antibiotic screening purposes, among others.

### SUMMARY OF THE INVENTION

The current disclosure relates, at least in part, to discovery and use of a phage-assisted continuous evolution (PACE)-compatible selection for orthogonal translation, which was successfully employed to identify ribosomal sequences possessing enhanced activity (e.g., increased translational activity), as compared to wild-type ribosome sequences. The disclosure therefore provides, among other aspects, a number of evolved rRNA sequences, which were remarkably identified to possess enhanced translation activities: improved orthogonal-ribosome binding site (o-RBS) and orthogonal anti-ribosome binding site (o-antiRBS) sequences; and host cells possessing deletion or disruption of ribosome hibernation promoting factor (HPF), which were herein identified to exhibit enhanced propagation of selection phage constructs. New transgenic organisms harboring the heterologous ribosomes and operons of the instant disclosure are also provided.

In one aspect, the instant disclosure provides a synthetic variant 16S ribosomal RNA (rRNA) that includes one or more of the following mutations: U409C, C440U, U904C, A906G, C1098U, G1415A and/or G1487A, where residue numbering is relative to the E. coli 16S rRNA sequence of SEQ ID NO: 40.

In one embodiment, the one or more mutations is present in a 16S rRNA sequence of E. coli, S. enterica, C. freundii, K. aerogenes, K. pneumoniae, K. oxytoca, E. cloacae, S. marcescens, P. mirabilis, P. stuartii, V. cholerae, A. macelodii, M. minitulum, P. aeruginosa, A. baumannii, A. faecalis, B. pertussis, B. cenocepacia, N. gonorrhoeae, M. ferrooxydans or C. crescentus.

In certain embodiments, the variant 16S rRNA includes U409C and G1487A mutations.

Another aspect of the disclosure provides a host cell that includes a variant 16S rRNA sequence as disclosed herein.

An additional aspect of the instant disclosure provides a host cell that includes a nucleic acid sequence having a non-host cell 16S ribosomal RNA (rRNA) variant sequence that includes one or more of the following mutations: U409C, C440U, U904C, A906G, C1098U, G1415A and/or G1487A, where residue numbering is relative to the E. coli 16S rRNA sequence of SEQ ID NO: 40.

In certain embodiments, the non-host cell is Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii or Shigella spp. (e.g., Shigella flexneri, Shigella dysenteriae, Shigella sonnei, Shigella boydii).

In some embodiments, the non-host cell is a commensal microbe. In a related embodiment, the commensal microbe is of one or more of the following phylum/phyla: Firmicutes, Bacteroidetes, Bifidobacteria, Eubacteria, Ruminococcus, Lactobacillus, Peptococcus, Proteobacteria, Verrumicrobia, Actinobacteria, Fusobacteria, Cyanobacteria, and a combination of phyla thereof.

In certain embodiments, the nucleic acid sequence that includes a non-host cell 16S ribosomal RNA (rRNA) variant sequence further includes intergenic sequences. Optionally, the intergenic sequences are host cell intergenic sequences.

In some embodiments, the non-host cell 16S rRNA variant sequence further includes an o-antiRBS sequence.

In one embodiment, the host cell further includes a nucleic acid sequence encoding for S20, S16, S1 and/or S15 r-protein(s) of the non-host cell.

In certain embodiments, translational output of the host cell that includes the variant 16S rRNA sequence is increased as compared to a control host cell that includes a wild-type 16S rRNA. Optionally, translational output is increased by at least 10% relative to the appropriate control.

In some embodiments, the host cell is Escherichia coli. Optionally, the host cell is an E. coli strain that includes a genomic deletion for rRNA sequences. Optionally the E. coli strain further includes a counter-selectable plasmid that includes E. coli rRNA sequences. Optionally, the E. coli strain is S1021, S2057, S2060, S3300, S3301, S3302, S3303, S3314, S3317, S3318, S3319, S3320, S3322, S3485 or S3489.

In certain embodiments, the host cell is Bacillus subtilis. Optionally, the host cell is a B. subtilis strain that includes a genomic deletion for rRNA sequences. Optionally, the host cell further includes a counter-selectable plasmid that includes B. subtilis rRNA sequence.

Another aspect of the instant disclosure provides a nucleic acid construct that includes an orthogonal anti-ribosome binding site (o-antiRBS) sequence of SEQ ID NOs: 8-10.

In one embodiment, the nucleic acid construct includes a 16S ribosomal RNA (rRNA) sequence. Optionally, the nucleic acid construct further includes 23S and/or 5S ribosomal RNA (rRNA) sequence. Optionally, the nucleic acid construct further includes phage genes. Optionally, the nucleic acid construct includes a sequence of SEQ ID NOs: 97 or 98.

An additional aspect of the instant disclosure provides a nucleic acid construct that includes an orthogonal-ribosome binding site (o-RBS) sequence of SEQ ID NO: 7.

In certain embodiments, the nucleic acid construct includes a gIII sequence. Optionally, the nucleic acid construct includes a sequence of SEQ ID NOs: 89, 91 or 92.

Another aspect of the instant disclosure provides a first nucleic acid construct that includes an orthogonal anti-ribosome binding site (o-antiRBS) sequence of SEQ ID NOs: 8-10 and a second nucleic acid construct that includes an orthogonal-ribosome binding site (o-RBS) sequence of SEQ ID NO: 7.

A further aspect of the instant disclosure provides a host cell that includes one or more nucleic acid constructs of SEQ ID NOs: 14-18. Optionally, at least one of the one or more nucleic acid constructs includes a non-host cell 16S rRNA sequence.

An additional aspect of the instant disclosure provides a host cell that includes a deletion or disruption of ribosome hibernation promoting factor (HPF) in the host cell genome and a nucleic acid sequence that includes a non-host cell ribosomal RNA (rRNA) sequence.

In certain embodiments, the host cell includes a variant 16S ribosomal RNA (rRNA) of the instant disclosure.

In some embodiments, the host cell harbors one or more nucleic acid construct(s) of the instant disclosure.

In one embodiment, the host cell is Escherichia coli. Optionally, the E. coli strain is S3300, S3314, S3317, S3322, S3485 or S3489.

In some embodiments, propagation of an orthogonal translation system in the host cell is improved by at least 100-fold (optionally by at least 3000-fold), as compared to an appropriate control host cell that possesses genomic ribosome hibernation promoting factor (HPF).

In certain embodiments, the host cell includes one or more of SEQ ID NOs: 89, 91, 92, 97 and/or 98.

Another aspect of the instant disclosure provides a nucleic acid construct that includes a truncated 16S ribosomal RNA (rRNA).

In certain embodiments, the nucleic acid construct includes one or more of SEQ ID NOs: 105-113.

A further aspect of the instant disclosure provides a host cell that includes a nucleic acid construct of the instant disclosure having a non-host cell truncated 16S ribosomal RNA (rRNA). Optionally, the nucleic acid construct includes an E. coli 16S rRNA.

In some embodiments, the host cell possesses o-rRNA-mediated translation activity that is retained or enhanced relative to an appropriate control host cell. Optionally, the host cell possesses o-rRNA-mediated translation activity levels of at least 80% that of an appropriate control host cell (i.e., a corresponding host cell having a full-length 16S o-rRNA).

Another aspect of the instant disclosure provides a nucleic acid construct that includes an orthogonal anti-ribosome binding site (o-antiRBS) and a 16S ribosomal RNA (rRNA) sequence. Optionally, the nucleic acid construct further includes 23S and/or 5S ribosomal RNA (rRNA) sequence. Optionally, the nucleic acid construct further includes phage genes. Optionally, the nucleic acid construct further includes one or more of SEQ ID NOs: 85-87.

A further aspect of the instant disclosure provides a nucleic acid construct that includes an orthogonal-ribosome binding site (o-RBS) sequence, an intein sequence, and a gIII sequence.

In certain embodiments, the intein sequence includes or is a GGS2 linker sequence (SEQ ID NO: 83), a MBP sequence (SEQ ID NO: 84) and/or a dT7RNAP sequence (SEQ ID NO: 114). Optionally, the nucleic acid construct includes SEQ ID NO: 93.

Another aspect of the instant disclosure provides a host cell that includes a nucleic acid construct of the instant disclosure.

An additional aspect of the instant disclosure provides a method for identifying in a host cell a non-host cell ribosomal RNA (rRNA) possessing enhanced translation activity, the method involving: (a) performing phage-assisted continuous directed evolution upon a population of host cells, where each host cell harbors: (i) a first nucleic acid construct that includes an orthogonal anti-ribosome binding site (o-antiRBS) and a 16S ribosomal RNA (rRNA) sequence (optionally, also including 23S and/or 5S ribosomal RNA (rRNA) sequence, phage genes, and/or one or more of SEQ ID NOs: 85-87); and (ii) a second nucleic acid construct that includes an orthogonal-ribosome binding site (o-RBS) sequence, an intein sequence, and a gIII sequence (optionally where the intein sequence is or includes a GGS2 linker sequence, a maltose binding protein (MBP) sequence and/or a dT7RNAP sequence, optionally where the nucleic acid construct includes SEQ ID NO: 93): (b) selecting for host cells that exhibit increased phage titer, as compared to a starting host cell; and (c) identifying a non-host cell ribosomal RNA (rRNA) sequence of a selected host cell of step (b), thereby identifying in a host cell a non-host cell ribosomal RNA (rRNA) that possesses enhanced translation activity.

A final aspect of the instant disclosure provides a method for enhancing non-host cell protein synthesis in a host cell, the method involving introducing a non-host cell translation system that includes a 16S rRNA sequence of SEQ ID NOs: 13, 15, 17, 19, 21, 23, 27, 31, 34 and/or 41-82 to the host cell, where non-host cell protein synthesis is enhanced in the host cell relative to an appropriate control (i.e., a host cell harboring a non-host cell translation system that does not include a 16S rRNA sequence of SEQ ID NOs: 13, 15, 17, 19, 21, 23, 27, 31, 34 and 41-82), thereby enhancing non-host cell protein synthesis in the host cell.

### Definitions

The term “host cell” is used herein to denote any cell, wherein any foreign or exogenous genetic material has been introduced. In its broadest sense, “host cell” is used to denote a cell which has been genetically manipulated. In certain embodiments, “host cell” refers to a microbe, optionally a prokaryotic cell, optionally a tractable prokaryotic cell (e.g., E. coli, B. subtilis, etc.).

As used herein, “heterologous sequence” or “heterologous protein” (e.g., heterologous ribosome) means any sequence or protein other than the one that naturally occurs within a host cell (optionally, in a host cell that has not been genetically modified). In certain embodiments, a heterologous sequence or protein is one for which a corresponding homologous sequence or protein exists within an unmodified host cell.

As used herein, the term “pathogenic microbe” refers to a biological microorganism that is capable of producing an undesirable effect upon a host animal, and includes, for example, without limitation, bacteria, viruses, bacterial spores, molds, mildews, fungi, and the like. This includes all such biological microorganisms, regardless of their origin or of their method of production, and regardless of whether they exist in facilities, in munitions, weapons, or elsewhere. In certain embodiments, the pathogenic microbe of the instant disclosure is a pathogenic bacteria.

As used herein, the term “commensal microbe” refers to a biological microorganism that lives on or in another organism without causing harm. A commensal microbe may refer, without limitation, to bacteria, viruses, fungi, and the like. The term therefore includes all such biological microorganisms, regardless of their origin or of their method of production, and regardless of where they exist. In certain embodiments, the commensal microbe of the instant disclosure is a commensal bacteria.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “homologous sequence” is meant a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant disclosure (e.g., 99%, 98%, 97%, 96%, 95%, 94%, 93%, 92%, 91%, 90%, 89%, 88%, 87%, 86%, 85%, 84%, 83%, 82%, 81%, 80% etc.). Indeed, design and use of the nucleotide sequences of the instant disclosure contemplates the possibility of using nucleotide sequences that are, e.g., only 99%, 98%, 97%, 96%, 95%, 94%, 93%, 92%, 91%, 90%, 89%, 88%, 87%, 86%, 85%, 84%, 83%, 82%, 81%, 80% etc. homologous to nucleotide sequences recited herein. Indeed, it is contemplated that nucleotide sequences with insertions, deletions, and single point mutations relative to the specific sequences disclosed herein can also be effective, e.g., for use in nucleic acid constructs (and in certain embodiments, in encoded polypeptide sequences) of the instant disclosure. In addition, it is expressly contemplated that nucleotide and/or amino acid sequences with analog substitutions or insertions can also be employed.

Sequence identity may be determined by sequence comparison and alignment algorithms known in the art. To determine the percent identity of two nucleic acid sequences (or of two amino acid sequences), the sequences are aligned for comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, a gapped alignment, the alignment is optimized by introducing appropriate gaps, and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25 (17): 3389-3402. In another embodiment, a global alignment the alignment is optimized by introducing appropriate gaps, and percent identity is determined over the entire length of the sequences aligned. (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1G show the development of a phage-assisted continuous evolution (PACE)-compatible selection for orthogonal translation. FIG. 1A shows a schematic representation of an orthogonal rRNA-dependent PACE selection. An engineered M13 bacteriophage (selection phage: SP) encodes the o-rRNA operon in place of gIII. Upon infection, functional orthogonal ribosomes efficiently translate gIII from the accessory plasmid (AP), yielding infectious phage progeny. Efficient o-rRNA diversification is implemented via a mutagenesis plasmid (MP). FIG. 1B shows AP and SP designs used in directed evolution campaigns. FIG. 1C shows a comparison of native and orthogonal RBS/antiRBS pairs used in this study. Sequences for wt RBS (SEQ ID NO: 1), wt antiRBS (SEQ ID NO: 2), o-RBSB (SEQ ID NO: 3), o-antiRBS_{B} (SEQ ID NO: 4), o-RBS_{lib} (SEQ ID NO: 5), o-antiRBS_{lib} (SEQ ID NO: 6), o-RBS_{H3} (SEQ ID NO: 7), o-antiRBS_{H3} (SEQ ID NO: 8), o-antiRBS_{H3-1} (SEQ ID NO: 9) and o-antiRBS_{H3-2} (SEQ ID NO: 10), are shown.

FIG. 1D shows a preliminary analysis of o-rRNA-dependent phage production under low (0 mM IPTG) or high (1 mM IPTG) mRNA concentrations using AP1H3. FIG. 1E shows the discovery of novel o-antiRBS variants under continuous culturing conditions using a degenerate library in the SP-borne o-rRNA. FIG. 1F shows a schematic representation of known ribosome hibernation factors. FIG. 1G shows a comparison of phage enrichment assays using the constitutive AP2H3 (top) in wild-type host (S2060) or host cells where ribosome hibernation factors have been deleted: hibernation promoting factor (Δhpf), ribosome modulation factor (Δrmf), ribosome associated inhibitor A (ΔraiA), or ribosomal silencing factor S (ΔrsfS). All data reflects the mean and standard deviation of 1-3 biological replicates.

FIGS. 2A to 2G show the identification of an optimal o-RBS/o-antiRBS system for SP-borne o-rRNAs. FIG. 2A shows a degenerate 47 (16,384) member library of RBS variants (o-RBS_{lib}, FIG. 1C above) was introduced to E. coli S2060 cells. The resultant cells were infected by phagemids (PDs) carrying the resistance gene for chloramphenicol (cat) and packed using a cognate helper phage (HP). APs encoding o-RBSs with significant crosstalk with the host's translational machinery would lead to gIII expression, rendering their hosts uninfectable and sensitive to chloramphenicol. O-RBSs with high orthogonality would allow phagemid infection and protection against chloramphenicol. FIG. 2B shows Sanger sequencing of 96 colonies yielded 33 unique o-RBS sequences, where the number following each sequence indicates frequency of occurrence. The seven most abundant variants (highlighted) were further characterized. FIG. 2C shows that to discover cognate o-antiRBSs, SPs bearing a degenerate library of 46 (4,096) antiRBS variants (o-antiRBS_{lib}, FIG. 1C above) were used to transform E. coli host cells that carry APs encoding each of the seven discovered o-RBSs. Functional o-antiRBS sequences should efficiently translate gIII, yielding pIII and giving rise to progeny phage. FIG. 2D shows that the RBS variant H3 (o-RBS_{H3}, FIG. 1C above) provided the most efficient propagation (highest titers) post infection. FIG. 2E shows that sequencing of clonal phage plaques identified up to 8 unique o-antiRBSs for each o-RBS sequence. For o-RBS_{H3}, the CTTGTA sequence (o-antiRBS_{H3}, FIG. 1C above) occurred with the highest frequency. FIG. 2F shows the effect of spacer sequence of o-antiRBS that was investigated using an orthogonal sfGFP reporter. The four base pair spacer sequence was found to be optimal, and was used for all further studies of the instant disclosure. FIG. 2G shows the experimental validation of two new o-antiRBSs discovered through continuous culturing (FIG. 2C above). Data reflects the mean and standard deviation of 3 biological replicates.

FIGS. 3A to 3F show the establishment of EP-SP correspondence via E. coli 16S rRNA truncation analysis. FIG. 3A shows the nucleotide conservation of the 16S rRNA which was used to guide truncated rRNA studies of the instant disclosure. The structure was generated via Ribovision (Bernier et al., 2014). FIG. 3B shows a composite of tested 16S rRNA truncations binned by their effects on orthogonal sfGFP reporter translation. Variants with sfGFP output below: 25% were considered “inactive”. FIG. 3C shows the key deletions used in the SP analysis as mapped on the E. coli 16S rRNA secondary structure. FIG. 3D shows that the single and double 16S rRNA truncations variably affected orthogonal GFP reporter translation, providing a gradient of activities for SP-based analyses. Data were normalized to untruncated E. coli 16S o-rRNA. FIG. 3E shows enrichment assays of SPs encoding full-length and truncated E. coli 16S o-rRNAs. FIG. 3F shows plaque assays that demonstrated the relationship between 16S o-rRNA activity and plaque formation. Labels indicate the truncation and activity in orthogonal GFP reporter translation relative to the untruncated 16S E. coli o-rRNA. Data reflect the mean and standard deviation of 1-11 biological replicates.

FIGS. 4A to 4L show the design and validation of an orthogonal translation-based genetic circuit for continuous directed evolution. Unless otherwise noted, all APs encode the wild-type replication initiation protein RepA (5-7 copies per cell) (Peterson and Phillips, 2008). RepA bearing the E93K mutation increased plasmid copy number to 26-29 copies per cell (Peterson and Phillips, 2008). FIG. 4A shows a comparison of insulated constitutive promoters (Davis et al., 2010) of varying strengths driving luxAB expression. FIG. 4B shows an overview of the expression plasmid (EP) and reporter plasmid (RP) used in luminescence assays. A schematic of the sequence-confirmed recombined phage (RC) referenced in FIG. 4H below is also included.

FIG. 4C shows a comparison of luminescence assays using a luciferase reporter with o-RBS_{H3} and an E. coli o-ribosome EP. FIG. 4D shows that promoter strengths correlated with phage propagation, as demonstrated by a comparison of phage enrichment assays using the constitutive AP2H3 (FIG. 1B above) and SP_{H3}. FIG. 4E shows a comparison of the AP1 and AP2 architectures and the effect of HPF deletion on SP propagation. FIG. 4F shows that O-rRNA-dependent SP propagation was severely attenuated in late stationary phase host cells (under low lagoon flowrates). FIG. 4G shows a comparison of phage enrichment assays using AP2_{H3} (FIG. 1B above) in S3317 cells using various constitutive promoters. FIG. 4H shows phage enrichment assays using AP2H3 in S3489 cells using various constitutive promoters. S3489 cells were identical to S3317 but were deleted for the fhuA gene to protect host cells from infections by lytic bacteriophages (Killmann et al., 1995). FIG. 4I shows continuous propagation of SP_{H3-1} using ^{ProA}AP2_{H3} (RepA E93K: 26-29 copies per cell (Davis et al., 2010)) in S3317 cells under high mutagenesis conditions using MP6 (Badran and Liu, 2015). M13-like recombinants developed after 28 h of continuous culture (indicated by *), which was attributed to high AP copy number. FIG. 4J shows PACE of SP_{H3-1} using proC AP2_{H3} (wt RepA: 5-7 copies per cell) in S3317 cells showed high phage titers at low lagoon flow rates (t=0-50 h: lagoons 3 and 4) but SP washout occurred at high lagoon flow rates (t=0-50 h: lagoons 1 and 2). The combination of wt RepA with proC promoter in this continuous evolution experiment should generate pIII transcript levels comparable to those in FIG. 4I above while maintaining a lower number of AP2_{H3} to ameliorate AP/SP recombination. Further reducing promoter strength to proA (t=50 h) resulted in M13-like recombinant phages by t=70 h. Sequencing analysis revealed that recombination occurred in the rmB terminator in AP2_{H3}, yielding a recombinant SP (RC in FIG. 4B above) that retained the o-antiRBS_{H3-1} sequence yet additionally integrated the AP-borne gIII cassette, controlled by o-RBS_{H3}. FIG. 4K shows that to limit this recombination, two synthetic terminator sequences were evaluated against the rmnB T1 terminator (Reynolds et al., 1992) in AP2_{H3} in phage enrichment assays. The T0 (21 imm)+BS7 dual terminator (McDowell et al., 1994) was found to provide the highest enrichment of SP_{H3} titers, and this final architecture is referred to as AP3 (FIG. 1B above). FIG. 4L shows the results of phage enrichment assays using AP3_{H3} in S3489 cells using various constitutive promoters. Data reflects the mean and standard deviation of 1-8 biological replicates.

FIGS. 5A to 5E show the continuous directed evolution of orthogonal ribosomes. FIG. 5A shows the starting o-ribosome activity of E. coli (Ec), P. aeruginosa (Pa) and V. cholerae (Vc) o-rRNAs, quantified using sfGFP production. FIG. 5B shows phage enrichment assays of SP_{Ec}, SP_{Pa}, and SP_{Vc} in S3489 cells using APs encoding promoters of decreasing strength. FIG. 5C shows phage enrichment assays of SP_{Ec}, SP_{Pa}, and SP_{Vc} in S3489 cells encoding variable inserts within the intein-proBAP_{H3} architecture: GGS2 linker, MBP and dT7RNAP. FIG. 5D shows a summary of PACE evolution trajectories. In the first trajectory, oRibo-PACE was carried out in three segments (segments 1→2→3). In the second trajectory, a shorter o-Ribo-PACE campaign was carried out in two segments (segments 1→4). In all segments, high levels of mutagenesis (MP6) (Badran and Liu, 2015a)) were induced. Phage titers sampled during o-ribosome evolutions and lagoon flowrates are shown on the bottom. FIG. 5E shows the average number of mutations per sequenced clone was highest in SP-borne o-rRNA derived from V. cholerae, followed by that of E. coli, while o-ribosome from P. aeruginosa on average had the lowest number of mutations at the end of each PACE segment. Data reflect the mean and standard deviation of 1-8 biological replicates.

FIGS. 6A to 6I show the design and validation of a split-intein pIII AP for continuous directed evolution. FIG. 6A shows the schematic representation of a split-intein PACE selection. Functional orthogonal ribosomes encoded by the SP must efficiently translate an intein-gIII transcript from the accessory plasmid (AP), which undergoes trans-splicing to produce functional pIII. FIG. 6B shows lengths of genes that were used as the “insert” in FIG. 6A. In all cases, prefix “d” indicates that the enzymatic activity has been inactivated by substitution at a catalytic residue. FIG. 6C shows overnight enrichment assays of SP_{H3-1} in S3489 using AP_{H3} VS. intein AP_{H3} with a GSS2 insert and driven by indicated promoters of varying strengths. FIG. 6D shows phage enrichment assay's of SP_{H3-1} in S3489 using ^{intein-proB}AP3_{H3} and insertions of various lengths. FIG. 6E shows a comparison of phage enrichment assays of SP_{Ec} starting sequences and after S1 and S2 of directed evolution in S3489 with AP3_{H3} and ^{intein}AP_{H3} variants. FIG. 6F shows a comparison of phage enrichment assays of SP_{Pa} starting sequences and after S1 and S2 of directed evolution in S3489 with AP3_{H3} and intein AP_{H3} variants. FIG. 6G shows a comparison of phage enrichment assays of SP_{Vc} starting sequences and after S1 and S2 of directed evolution in S3489 with AP3_{H3} and intein AP_{H3} variants. FIG. 6H shows that O-rRNA operons encoded in the SPs underwent truncations after 27-43 h of PACE, as shown by PCR products from amplifications of the entire rRNA operons throughout segment 1 (0-68 h). It was notable that loss of the 23S subunit in SP_{Pa} and SP_{Vc} occurred concurrently with truncation of the 5S subunit while SPEC retained its 5S subunit throughout the first segment. At the end of the segments 3 and 4, several clones of SP_{Ec} underwent further truncation of the remaining 5S subunit. The truncated o-RNAs in all SP populations retained the 16S rRNA 3′ processing sequence, highlighting its important role in base-paring with the 16S rRNA 5′ leader sequence (Brosius et al., 1981). A similar rRNA truncation point was found in prior attempts to discover a minimal o-rRNA (An and Chin, 2009).

FIG. 6I shows a pairwise sequence alignment of partial 16S rRNAs from E. coli, P. aeruginosa, and V. cholerae, noting consensus mutations in individual organisms and positions that were mutated independently in multiple organisms. Respective sequence segments shown are: E. coli wt residues 391-460 (SEQ ID NO: 11), P. aeruginosa wt residues 385-454 (SEQ ID NO: 12), V. cholerae wt residues 391-460 (SEQ ID NO: 14), E. coli wt residues 871-940 (SEQ ID NO: 16), P. aeruginosa wt residues 865-934 (SEQ ID NO: 18), V. cholerae wt residues 871-940 (SEQ ID NO: 20), E. coli wt residues 1061-1130 (SEQ ID NO: 22), P. aeruginosa wt residues 1055-1124 (SEQ ID NO: 24), V. cholerae wt residues 1061-1130 (SEQ ID NO: 25), E. coli wt residues 1381-1450 (SEQ ID NO: 26), P. aeruginosa wt residues 1375-1444 (SEQ ID NO: 28), V. cholerae wt residues 1382-1451 (SEQ ID NO: 29), E. coli wt residues 1451-1520 (SEQ ID NO: 30), P. aeruginosa wt residues 1445-1514 (SEQ ID NO: 32) and V. cholerae wt residues 1452-1521 (SEQ ID NO: 33). Indicated variant sequence segments of those displayed are: P. aeruginosa A434U (SEQ ID NO: 13), V. cholerae U409C (SEQ ID NO: 15), E. coli U904C, A906G (SEQ ID NO: 17), P. aeruginosa A900G (SEQ ID NO: 19), V. cholerae U904C, A906G (SEQ ID NO: 21), E. coli C1098U (SEQ ID NO: 23), E. coli G1415A (SEQ ID NO: 27), E. coli G1487A (SEQ ID NO: 31) and V. cholerae G1487A (SEQ ID NO: 34).

FIGS. 7A to 7F show shared consensus mutations identified in 16S rRNAs following continuous evolution. FIG. 7A shows an overview of consensus rRNA mutations observed in oRibo-PACE for E. coli with selection. Values represent % of sequenced clones from each segment. FIG. 7B shows an overview of consensus rRNA mutations observed in oRibo-PACE for P. aeruginosa with selection. FIG. 7C shows an overview of consensus rRNA mutations observed in oRibo-PACE for V. cholerae with selection. FIG. 7D shows Shannon entropy values as determined for positions where consensus mutations were discovered in oRibo-PACE. FIG. 7E shows that phylogenetic divergence at positions mutated during oRibo-PACE (outlined squares) showed no correlation between a discovered o-rRNA mutation and nucleotide conservation at that position. Shannon entropy values and nucleotide abundance were both obtained from RiboVision (Bernier et al. 2014). FIG. 7F shows consensus rRNA mutations discovered in PACE and their locations on the ribosome. Most ribosomal proteins have been omitted for clarity. A close-up view of h37 in the 16S rRNA and the C1098U mutation in relation to ribosomal protein uS2 is shown. Close up locations of U409C (V. cholerae only) and C440U (P. aeruginosa only) mutations in relation to ribosomal protein uS4 are also shown. And a close-up view of mutations discovered by >2 rRNA evolution campaigns on h27 and h44 in relation to uS12 is shown. For all parts, images were generated from a 2.8-A Thermus thermophilus 70S ribosome structure (PDB 4v51 (Selmer et al., 2006)). All positions are numbered using E. coli 16S rRNA nomenclature.

FIGS. 8A to 8J show an analysis of evolved o-rRNA activities and transplantation of consensus mutations in heterologous o-rRNAs. FIG. 8A shows a schematic of luminescence assays: upon induction of the o-rRNA EP, o-ribosomes are produced and translate an orthogonal luxAB transcript to produce luminescence. FIG. 8B shows the evaluation of oRibo-PACE-evolved E. coli o-rRNAs using an orthogonal luciferase reporter. For all luminescence assays, o-ribosome activities are expressed as % of starting E. coli o-ribosome. FIG. 8C shows the evaluation of oRibo-PACE-evolved P. aeruginosa o-rRNAs using an orthogonal luciferase reporter. For all luminescence assays, o-ribosome activities are expressed as % of starting E. coli o-ribosome. FIG. 8D shows the evaluation of oRibo-PACE-evolved V. cholerae o-rRNAs using an orthogonal luciferase reporter. For all luminescence assays, o-ribosome activities are expressed as % of starting E. coli o-ribosome. FIG. 8E shows cell burden: upon induction of the EP, cellular resources are diverted to the production of o-ribosomes, which are devoted to translation of the orthogonal luciferase transcript and cannot produce host proteins essential for host survival. Consequently, induction of o-ribosome production exerts a metabolic burden on the E. coli host (Orelle et al. 2015), as manifested by changes in its doubling time. FIG. 8F shows results of quantifying the burden of oRibo-PACE-evolved E. coli o-rRNAs on S3489 cell doubling time, with the starting variant (st) provided for comparison. FIG. 8G shows results of quantifying the burden of oRibo-PACE-evolved P. aeruginosa o-rRNAs on S3489 cell doubling time, with the starting variant (st) provided for comparison. FIG. 8H shows results of quantifying the burden of oRibo-PACE-evolved V. cholerae o-rRNAs on S3489 cell doubling time, with the starting variant (st) provided for comparison. FIG. 8I shows that through analysis of consensus mutations discovered through oRibo-PACE, 12 mutations were transplanted into unrelated heterologous o-rRNAs from Salmonella enterica and investigated using the orthogonal luciferase reporter. FIG. 8J shows the result of the same 12 mutations transplanted into unrelated heterologous o-rRNAs from Serratia marcescens, investigated using the orthogonal luciferase reporter. The combination of the two mutations U409C and G1487A showed the greatest improvement in both o-rRNAs of above FIGS. 8I and 8J. Data reflect the mean and standard deviation of 6-32 biological replicates.

FIGS. 9A to 9J show in-depth characterization of evolved O-ribosome activities. FIG. 9A shows a schematic of o-rRNA variants from each oRibo-PACE segment that were cloned into expression plasmids (EPs) and tested alongside reporter plasmids (RPs) of variable genes, RBSs, and context dependencies. FIG. 9B shows luminescence activity calculated at OD₆₀₀=0.15 plotted against host strain (S3489) doubling time for o-rRNA variants derived from each species corresponding to selections S1→S2, FIG. 9C shows luminescence activity calculated at OD₆₀₀=0.15 plotted against host strain (S3489) doubling time for o-rRNA variants derived from each species corresponding to selections S2→S3. FIG. 9D shows luminescence activity calculated at OD₆₀₀=0.15 plotted against host strain (S3489) doubling time for o-rRNA variants derived from each species corresponding to selections S1→S4. FIG. 9E shows results for select o-rRNA variants that were prioritized based on luminescence activity and evaluated for sfGFP production in the absence of cognate heterologous ribosomal proteins (r-proteins). FIG. 9F shows results for select o-rRNA variants that were prioritized based on luminescence activity and evaluated for sfGFP production in the presence of cognate heterologous ribosomal proteins (r-proteins). FIG. 9G shows incorporation of the ncAA Bock for select variants in the absence of cognate heterologous r-proteins. FIG. 9H shows incorporation of the ncAA Bock for select variants in the presence of cognate heterologous r-proteins. FIG. 9I shows that sfGFP yield through orthogonal translation and using either the B or H3 o-RBS showed comparable activities in most cases. FIG. 9J shows results obtained when consensus mutations U409C and G1487 discovered through oRibo-PACE were incorporated into rRNAs derived from phylogenetically divergent bacterial species, and evaluated for sfGFP production in the presence or absence of cognate heterologous r-proteins.

FIGS. 10A to 10G show complementation of SQ171 cells using evolved rRNAs. FIG. 10A shows a schematic representation of SQ171 complementation assays in which evolved RNA variants were engineered to encode wt-antiRBS to translate the cellular proteome necessary for the survival of the SQ171 host cells. FIG. 10B shows the evaluation of oRibo-PACE-evolved E. coli rRNAs using complemented SQ171 cell doubling time, with the starting variant bearing the wild-type antiRBS (wt) provided for comparison. FIG. 10C shows the evaluation of oRibo-PACE-evolved P. aeruginosa rRNAs using complemented SQ171 cell doubling time, with the starting variant bearing the wild-type antiRBS (wt) provided for comparison. FIG. 10D shows the evaluation of oRibo-PACE-evolved V. cholerae rRNAs using complemented SQ171 cell doubling time, with the starting variant bearing the wild-type antiRBS (wt) provided for comparison. FIG. 10E shows a comparison of complemented SQ171 strain doubling times using cognate 23S rRNA vs. E. coli-derived 23S rRNAs. Use of the E. coli 23S showed improved doubling time using both P. aeruginosa and V. cholerae 16S rRNAs, in agreement with the SP truncations during oRibo-PACE. The data of the instant disclosure therefore implement the E. coli 23S rRNA alongside P. aeruginosa and V. cholerae 16S rRNAs.

FIG. 10F shows a sequence comparison that identifies the location of a BsmI cleavage site in E. coli rRNAs that does not appear in corresponding P. aeruginosa and V. cholerae rRNAs. Displayed sequences are E. coli residues 1340-1379 (SEQ ID NO: 35), P. aeruginosa residues 1334-1373 (SEQ ID NO: 36) and V. cholerae residues 1341-1380 (SEQ ID NO: 37). FIG. 10G shows results obtained when complemented SQ171 strains encoding starting and evolved rRNAs in biological triplicate were PCR amplified using universal primers AB5606 (5′-cggtggagcatgtggttt-3′: SEQ ID NO: 38) and AB5113 (5′-acgccttgcttttcactttc-3′: SEQ ID NO: 39) to yield a ˜668 bp PCR product. This PCR product is then digested using BsmI (New England Biolabs®), which effectively identifies the rRNAs in SQ171 cells by either yielding two fragments (428 bp and 240 bp) to indicate an E. coli rRNA (as shown in FIG. 10F above) or no cleavage to indicate a heterologous rRNA. This analysis confirmed correct rRNA plasmid exchange in all benchmarked SQ171 cells.

FIGS. 11A to 11O show that evolved rRNAs supported proteome-wide translation at elevated levels. FIG. 11A shows a schematic representation where the o-RBS of oRibo-PACE-derived rRNA variants was substituted with the wild-type RBS, and used to complement SQ171 strains (resident plasmids cured by sucrose selection). FIG. 11B shows o-ribosome luminescence activity plotted against complemented SQ171 strain doubling times for all species corresponding to selections segment S1→S2, FIG. 11C shows luminescence activity plotted against complemented SQ171 strain doubling times for all species corresponding to selections segment S2→S3. FIG. 11D shows luminescence activity plotted against complemented SQ171 strain doubling times for all species corresponding to selections segment S1→S4. FIG. 11E shows results obtained when select rRNA variants were prioritized based on luminescence activity and evaluated for the cellular characteristic of electron transport chain function as assessed through cellular reductase activity. Data represents mean fluorescence intensity (MFI) with error shown as standard deviation of three biological replicates. FIG. 11F shows results obtained when select rRNA variants were prioritized based on luminescence activity and evaluated for the cellular characteristic of membrane integrity as assessed through propidium iodide entry. Data represents mean fluorescence intensity (MFI) with error shown as standard deviation of three biological replicates. FIG. 11G shows that SQ171 strain sensitivity to the mistranslation-promoting aminoglycoside kanamycin negatively correlated with evolved o-ribosome activity. FIG. 11H shows that SQ171 strain sensitivity to the mistranslation-promoting aminoglycoside gentamicin negatively correlated with evolved o-ribosome activity. FIG. 11 shows that complemented SQ171 strains showed increased volume concomitant with observed increases of the population doubling time. FIG. 11J shows a schematic representation of the workflow used to analyze amino acid mistranslation rates through sfGFP purification and LC-MS/MS analysis. FIG. 11K shows the amino acid substitution frequency of select rRNA variants via sfGFP expression, shown as a % of total amino acid detected at a given position. Data reflects sfGFP purified from six pooled biological replicates. Each point represents an identified amino acid substitution. The grey bar represents average cellular amino acid mis-incorporation limits. FIG. 11L shows the structure of methionine (Met) and the methionine analogue L-azidohomoalanine (AHA), which was used to determine proteome-wide translation rate through unbiased cellular incorporation. FIG. 11M shows the mean slope of AHA incorporation calculated from 20-minute time course analysis. Data were normalized to mean slope of wild-type E. coli from each experimental run. FIG. 11N shows that complemented SQ171 cells showed similar reductase activity. FIG. 11O shows observed membrane integrity measurements during the AHA incorporation assay. Where relevant, data is normalized to activity of the starting E. coli o-rRNA activity. Starting rRNAs are shown as filled in bars or circles, whereas evolved variants are shown as borders only. Data reflect the mean and standard deviation of 1-72 biological replicates.

FIGS. 12A to 12H show an analysis of mistranslation SQ171 cells complemented with kinetically-enhanced rRNAs. FIG. 12A shows IC₅₀ values observed for the error-inducing aminoglycoside kanamycin for select E. coli and V. cholerae rRNA-complemented SQ171 strains. FIG. 12B shows IC₅₀ values observed for the error-inducing aminoglycoside gentamicin for select E. coli and V. cholerae rRNA-complemented SQ171 strains. FIG. 12C shows that following sfGFP analysis for misincorporation via protein purification and LC-MS/MS analysis, the correlation between kinetic o-ribosome translation activity and average amino acid substitution frequency was examined and plotted. O-ribosome activity was normalized to starting E. coli o-rRNA activity. Amino acid substitution frequency was calculated as the (%) substation abundance of sum of all peptides mapping to a specific residue in sfGFP. Data is shown as the mean and standard deviation of 3 biological replicates. FIG. 12D shows sites within sfGFP where substitutions were detected, displayed for tested E. coli-derived rRNAs in SQ171 strains. Each row corresponds to a unique strain and columns to individual residues of sfGFP (1-246 residues). FIG. 12E shows sites within sfGFP where substitutions were detected, displayed for tested V. cholerae-derived rRNAs in SQ171 strains. Each row corresponds to a unique strain and columns to individual residues of sfGFP (1-246 residues). FIG. 12F shows a comparison of observed amino acid substitutions and mRNA codon identities for select E. coli and V. cholerae-derived rRNAs in SQ171 strains. Color indicates (%) substitutions at a codon by a non-cognate amino acid. Each heatmap is labeled with the strain of E. coli-derived (top) or V. cholerae-derived (bottom) rRNA variant. FIG. 12G shows aggregated amino acid mis-incorporation for all select rRNA variants. FIG. 12H shows codon adaptation index of sfGFP for wild-type and evolved Ec and Vc variants.

FIG. 13 shows Sanger sequencing analysis of SPEC samples during four separate segments of PACE. Mutations are colored on the basis of the stage in which they first became fixed in the evolving o-rRNA pool. Numbers in parentheses indicate the number of independent plaques isolated that carry the identical mutation(s). ¹indicates that % o-rRNA activity of each SP mutant is presented in bar graph form in FIG. 6B above. ²indicates that doubling times (in mins) of SQ171 cells carrying EPs encoding evolved rRNA variants are presented in bar graph form in FIG. 8B above. ³indicates that doubling times (in mins) of E. coli host cells carrying EPs encoding evolved rRNA variants are presented in bar graph form in FIG. 6F above.

FIG. 14 shows Sanger sequencing analysis of SP_{Pa} samples during four separate segments of PACE. Mutations are colored on the basis of the stage in which they first became fixed in the evolving o-rRNA pool. Numbers in parentheses indicate the number of independent plaques isolated that carry the identical mutation(s). ¹indicates that % o-rRNA activity of each SP mutant is presented in bar graph form in FIG. 6C above. ²indicates that doubling times (in mins) of SQ171 cells carrying EPs encoding evolved rRNA variants are presented in bar graph form in FIG. 8C above. ³indicates that doubling times (in mins) of E. coli host cells carrying EPs encoding evolved rRNA variants are presented in bar graph form in FIG. 6G above.

FIG. 15 shows Sanger sequencing analysis of SP_{Vc} samples during four separate segments of PACE. Mutations are colored on the basis of the stage in which they first became fixed in the evolving o-rRNA pool. Numbers in parentheses indicate the number of independent plaques isolated that carry the identical mutation(s). ¹indicates that % o-rRNA activity of each SP mutant is presented in bar graph form in FIG. 6D above. ²indicates that doubling times (in mins) of SQ171 cells carrying EPs encoding evolved rRNA variants are presented in bar graph form in FIG. 8D above. ³indicates that doubling times (in mins) of E. coli host cells carrying EPs encoding evolved rRNA variants are presented in bar graph form in FIG. 6H above.

FIG. 16 shows genotypes of all bacterial strains of the instant disclosure.

### DETAILED DESCRIPTION OF THE INVENTION

The present disclosure is directed, at least in part, to discovery of an orthogonal ribosome dependent phage-assisted continuous evolution (oRibo-PACE) methodology, that enabled rapid directed evolution of rRNA towards researcher-defined activities. The disclosed system was employed herein to explore the interplay between translational kinetics and fidelity through the evolution of 16S rRNA from three bacterial species. Evolved rRNA mutants were characterized herein through variable reporter gene and context dependencies in an orthogonal translation system, and it was remarkably identified herein that two of three starting rRNA scaffolds evolved to achieve higher kinetic translation rates than those of wild-type E. coli rRNA in an E. coli host. Observed activities were found to function in a context-independent manner, when evaluated using variable reporter gene(s), ribosome-binding site(s) (RBS) and alongside cognate heterologous r-proteins, in some cases. These findings were then extended herein to generate and thereby provide cells harboring only evolved rRNA variants, which showcased elevated proteome-wide translation rates as compared to wild-type E. coli rRNA. Critically, evolved rRNAs catalyzed protein production with moderate reductions in translational fidelity, which has also highlighted a previously unrecognized relationship between enhanced translational kinetics and increased degrees of mistranslation.

Certain aspects of the instant disclosure therefore provide approaches and resultant compositions for identification of ribosomal RNAs possessing enhanced properties (e.g., increased translation activities). The approaches disclosed herein have leveraged strain engineering, orthogonal translation and phage-assisted continuous evolution to access improved and expanded translation capabilities in living cells. Among the compositions and methods disclosed herein are optimized RBS-antiRBS sequence pairs, improved heterologous rRNAs (identified via directed evolution), bacterial strains that exhibit improved ribosome activity through rRNA evolution and/or hibernation factor deletion, a split-intein based selection methodology, and extension of consensus mutations to phylogentically divergent rRNAs for similarly improved translation properties.

The compositions and methods disclosed herein offer a number of specific advantages:
(1) RBS-antiRBS pairs of sequences disclosed herein provide enhanced dynamic range of orthogonal translation (observed >70,000-fold enhancement of dynamic range).
(2) Deletion of host hibernation factors was identified to improve orthogonal translation.
(3) Development of a split-intein selection methodology disclosed herein allowed for artificially increasing open reading frame (ORF) length.
(4) The evolved rRNA sequences disclosed herein provide increased ribosome translation rates in vivo, of up to four-fold increases relative to wild-type sequences.
(5) The evolved ribosomes of the instant disclosure increase recombinant protein production by up to six-fold, relative to wild-type ribosomes.
(6) The evolved ribosomes of the instant disclosure provide improved non-canonical amino acid incorporation efficiency in vivo, by up to 40-fold over wild-type ribosomes (of particular use in synthesis of modified peptides, as non-canonical amino acids can provide for an expanded array of available modifications).
(7) The evolved ribosomes of the instant disclosure also mitigate some viability issues previously observed with orthogonal translation, by improving heterologous ribosome activity.
(8) Specific use of the improved ribosomes of the instant disclosure is contemplated for production of high-value/poor-yield recombinant biologics (e.g. recombinant insulin/erythropoietin).
(9) The improved ribosomes of the instant disclosure can also be used in generation of biologics with expanded genetic codes (e.g., antibodies with defined chemical side chains incorporated by ribosomes).
(10) The ribosomes of the disclosure can also be used in rapid in vitro (cell-free) translation diagnostics for infections/disease (e.g., used to make an in vitro translation system).

In bacteria, ribosome kinetics are considered rate-limiting for protein synthesis and cell growth. Increased ribosome kinetics may augment bacterial growth and increase overall protein yield, but whether this can be achieved by ribosome-specific modifications has previously remained unknown. The instant disclosure has remarkably revealed that 16S ribosomal RNAs (rRNAs) from Escherichia coli, Pseudomonas aeruginosa, and Vibrio cholerae can be effectively evolved towards enhanced protein synthesis rates. It has specifically been discovered herein that rRNA sequence origin significantly impacted evolutionary trajectory and generated RNA mutants with augmented protein synthesis rates in both natural and engineered contexts. Moreover, discovered consensus mutations disclosed herein can be ported onto phylogenetically divergent rRNAs, imparting improved translational activities. Finally, it has been demonstrated herein that increased translation rates in vivo coincided with reduced translational fidelity, and did not enhance bacterial population growth. Together, the findings of the instant disclosure have demonstrated that cellular protein synthesis rates can be natively optimized to balance trade-offs between protein synthesis kinetics and translational fidelity in living systems.

In nutrient-rich environments, bacterial growth rate is intimately linked to ribosome content and the corresponding translation rate (Scott et al., 2014). Given its critical role in modulating cellular growth rate, ribosome content of a cell is tightly regulated to mitigate over-commitment of resources (Serbanescu, Ojkic and Banerjee, 2020). Indeed, rRNAs and ribosomal proteins (r proteins) can make up approximately half of the total E. coli dry mass (Dennis, Ehrenberg and Bremer, 2004). Bacteria encoding more ribosomal RNA (rRNA) operons often show increased growth rates (Roller, Stoddard and Schmidt, 2016) and r-proteins are synthesized preferentially over all other proteins during exponential growth (Hui et al., 2015), suggesting that increases in the cellular commitment to ribosome production may dictate the growth rate of a cell. In addition to ribosome content, cellular translation rate may be influenced by a multitude of other factors, including the translation initiation efficiency (Saito, Green and Buskirk, 2020), aminoacylated tRNA abundance (Novoa et al., 2012), elongation factor availability (Klumpp et al., 2013), messenger RNA (mRNA) codon usage (Boel et al., 2016), and amino acid composition of the nascent polypeptide (Riba et al., 2019). Whereas mutations to rRNAs or r-proteins can enhance translational fidelity (Agarwal et al., 2015), reduce translational kinetics (McClory, Devaraj and Fredrick, 2014), endow antibiotic resistance (Bjorkman et al., 1999), or even affect ribosome assembly (Aleksashin et al., 2019), it has heretofore remained unknown whether the translation rate can be increased by ribosome-specific modifications.

Directed evolution of rRNAs (Neumann et al., 2010: Liu et al., 2018) towards new-to-nature bioactivities has highlighted plasticity in the cellular translation apparatus, indicating that evolution towards enhanced kinetics may be feasible. However, classical directed evolution has required extensive effort to maximize library diversity, fine-tune sequential selection conditions, and has also suffered from limitations in mutational spectrum and library transformation efficiencies (Bratulic and Badran, 2017). Given the large sequence space of an rRNA, there has been a long-felt need for powerful methods of directed evolution, to access improved kinetic translation capabilities. At the outset of the studies disclosed herein, it was identified that a high throughput methodology for directed evolution of rRNAs might therefore facilitate unbiased investigations of ribosomal translation and could enable novel biosynthetic capabilities. In particular, orthogonal translation systems, which create dedicated pools of researcher-controlled ribosomes that are decoupled from cellular viability (Rackham and Chin, 2005), have enabled the exploration of sequence-function relationships en route to novel bioactivities (Neumann et al., 2010), permitted investigations into mutation of sequence essential for cell function, and enabled discovery of novel ribosomal activities (Aleksashin et al., 2020).

Bolstered by this decoupled translation framework, the presently disclosed orthogonal ribosome dependent phage-assisted continuous evolution (oRibo-PACE) methodology was developed.

The instant disclosure has therefore yielded functionally enhanced ribosomes, including heterologous ribosomes, in E. coli. (with application to other microbes (e.g., B. subtilis) also expressly contemplated). Cumulatively, the instant disclosure also enables further generation of functionally enhanced ribosomes possessing new and specialized capabilities for synthetic translation. Heterologous rRNA-harboring genetic organisms have also been provided herein. Such organisms provide enhanced ribosomes and/or also allow for improved synthetic ribosome evolution. The compositions, methods and application(s) of the instant disclosure are considered in additional detail below.

rRNAs and r-Proteins

The E. coli ribosome is composed of two large particles, the 30S and the 50S subunits. The 30S subunit consists of a 16S rRNA molecule and 21 small ribosomal proteins (“r-proteins’). The 50S subunit is composed of two ribosomal RNA (rRNA) molecules, 23S and 5S rRNA, and 31 large ribosomal proteins.

Prokaryotic ribosomes are similar across species, but homology of individual ribosomal proteins diverges with phylogenetic distance. rRNAs are relatively few in number and yet play an important role in protein synthesis (Gutell et al., 1985, Prog. Nucleic Acid Res. Mol. Biol. 32:155-216). Ribosome assembly in bacteria is a tightly controlled process. For example, the synthesis of ribosomal components, rRNA and r-proteins, is coordinately regulated to ensure that these molecules are produced in the optimal stoichiometry. Protein-RNA interactions play important regulatory roles at several steps in this process. Synthesis of r-proteins is negatively regulated at the translational level by the binding of repressor r-proteins to specific sites in mRNA. As part of another regulatory step in the ribosome assembly process, certain r-proteins bind to rRNA, to initiate the ordered assembly of the ribosome. Binding of these r-proteins, termed “primary binders,” is required for the subsequent steps of ribosome assembly (Zengel & Lindahl, 1994, Prog. Nuc. Acid Res. Molec. Biol. 47:332-370).

The interaction of ribosomal proteins with RNA influences the synthesis of ribosomal proteins and their assembly into fully functional ribosomes. Ribosomal assembly in E. coli involves the coordinate expression of rRNA and r-proteins. Binding of certain ribosomal proteins to ribosomal RNAs (rRNAs) is necessary for the ordered assembly of fully functional ribosomes. In the course of assembly, a subset of ribosomal proteins, termed primary binding r-proteins, has been identified as binding rRNA directly, and as facilitating the binding of other ribosomal proteins.

rRNA and Construct Sequences

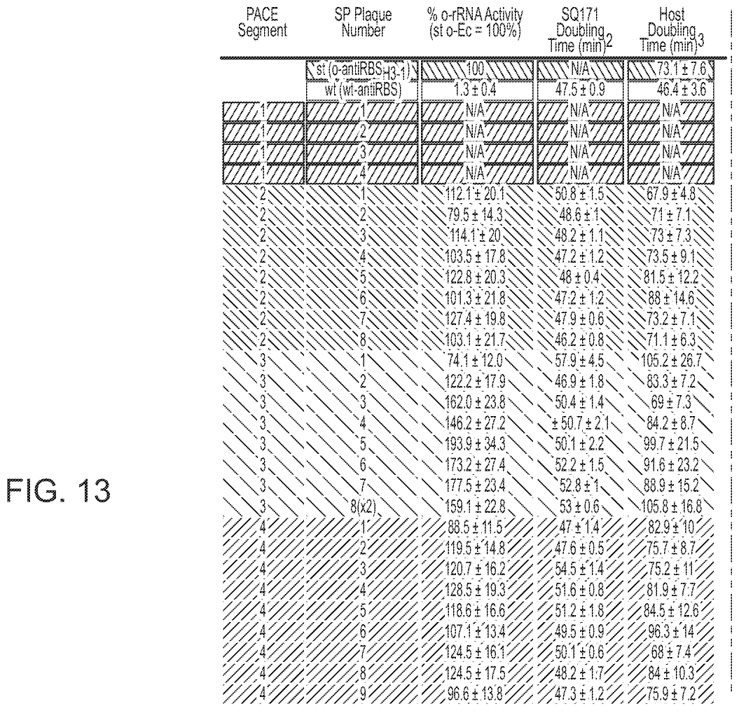

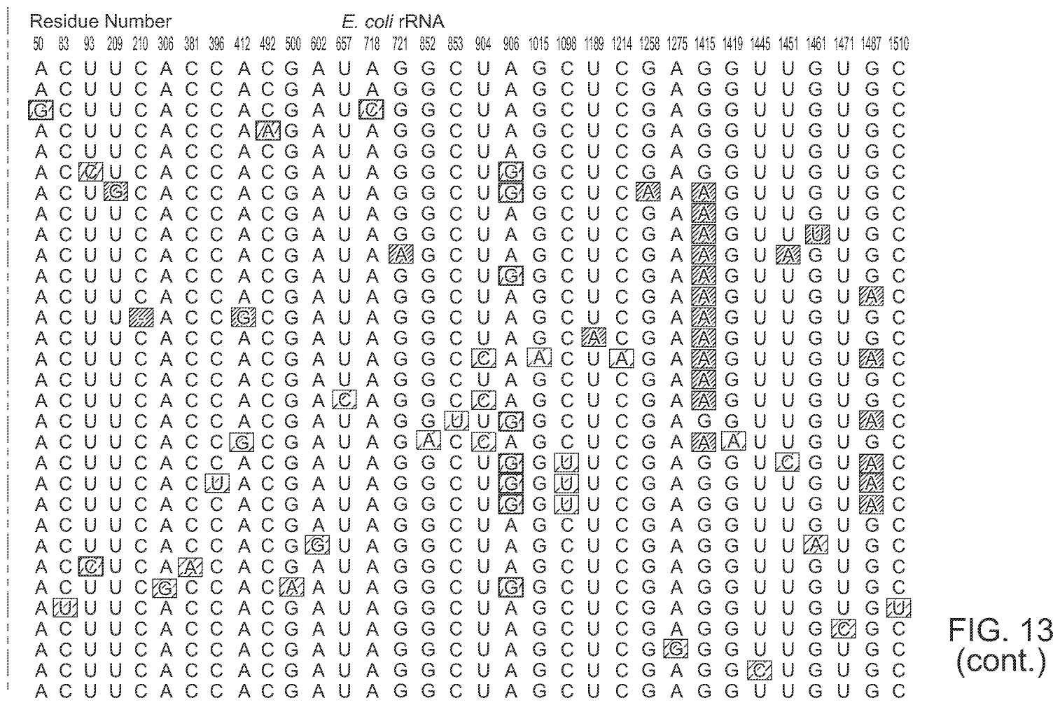

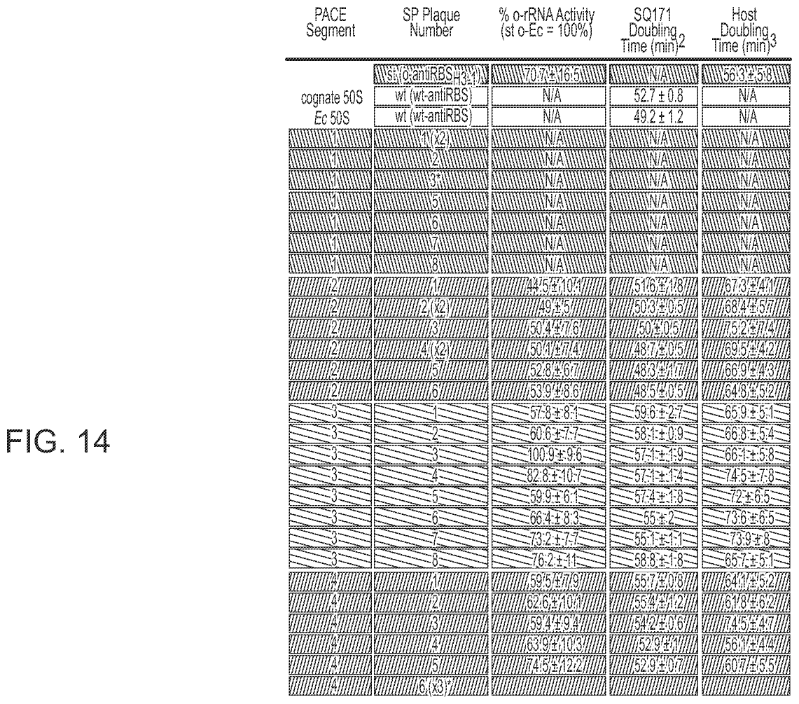

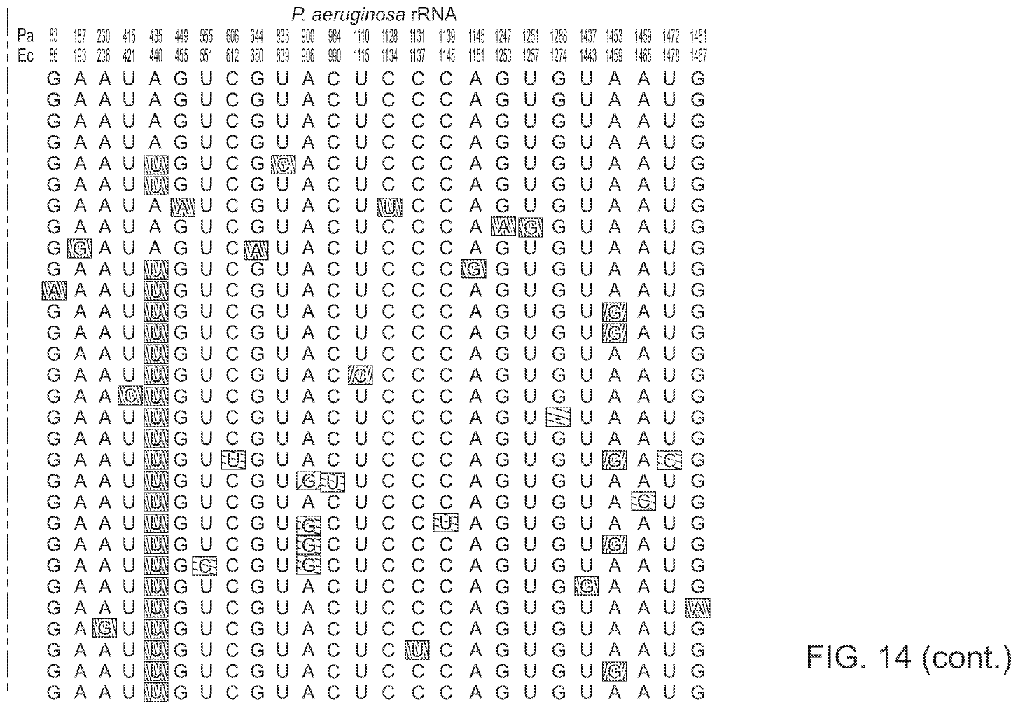

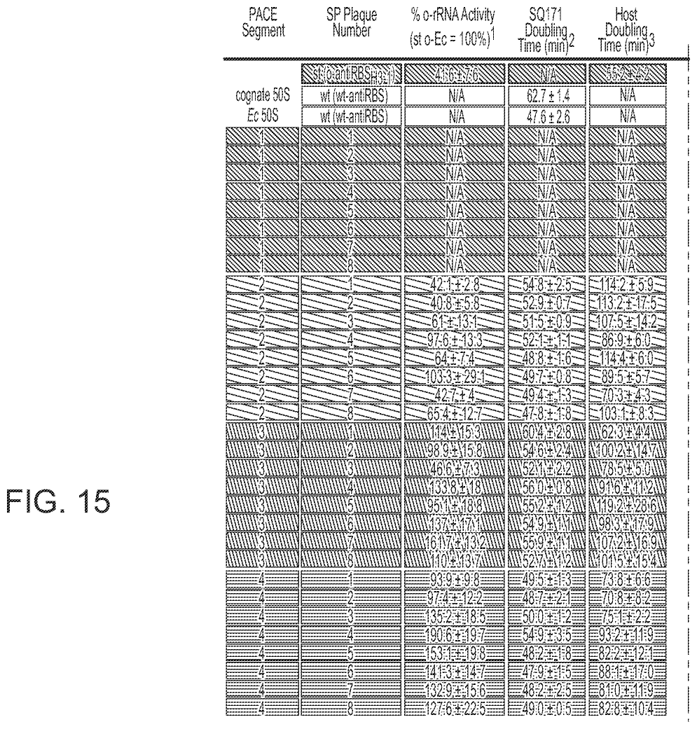

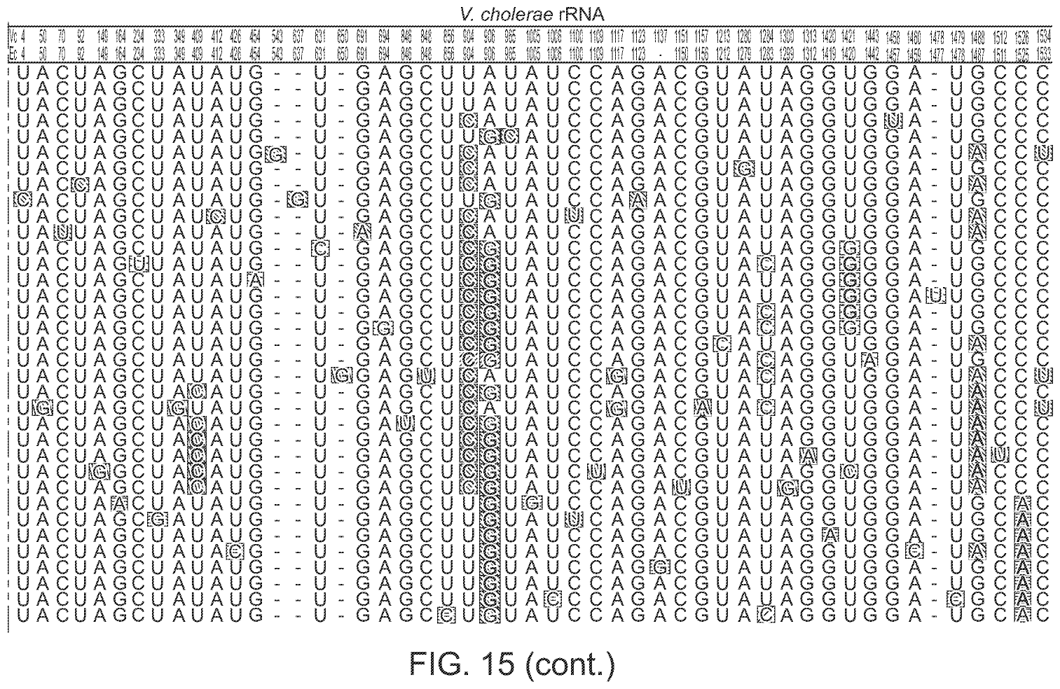

rRNA and reporter construct sequences of the instant disclosure are presented in the accompanying Sequence Listing, with Table I also presenting a description of each sequence of the Sequence Listing. The variant sequences set forth in FIGS. 13-15 are also noted as encompassed by the instant disclosure.

It is expressly contemplated that the rRNA and reporter construct sequences of the instant disclosure can also differ from any one of the nucleotide sequences of Table 1 and/or FIGS. 13-15 at a number (1, 2, 3, 4, 5, 6, 7, 8, 9, 10, etc.) of residues while still retaining the activities identified herein for such sequences. Thus, the instant disclosure also encompasses, e.g., a nucleotide sequence having at least 90% nucleotide sequence identity, e.g. 90, 91, 92, 93, 94, 95, 96, 97, 98, 99 or 100% identity, to the entire nucleotide sequence of any one of the nucleotide sequences of Table 1 and/or FIGS. 13-15, where a substitution of a uracil for any thymine of the nucleotide sequences of Table 1 and/or FIGS. 13-15 (when comparing aligned sequences) does not count as a difference.

The evolved ribosomes of the instant disclosure have been identified as providing improved non-canonical amino acid (ncAA) incorporation efficiency in vivo, as compared to non-evolved (i.e., wild-type) ribosomes. It is therefore contemplated herein that certain evolved ribosomes of the instant disclosure provide at least a two-fold improvement in ncAA incorporation efficiency (as measured, e.g., in a ncAA incorporation assay of Chatterjee et al.), as compared to a corresponding non-evolved and/or wild-type ribosome. Optionally, the evolved ribosomes of the instant disclosure provide at least a three-fold improvement in ncAA incorporation efficiency, optionally at least a four-fold improvement in ncAA incorporation efficiency, optionally at least a five-fold improvement in ncAA incorporation efficiency, optionally at least a six-fold improvement in ncAA incorporation efficiency, optionally at least a seven-fold improvement in ncAA incorporation efficiency, optionally at least an eight-fold improvement in ncAA incorporation efficiency, optionally at least a nine-fold improvement in ncAA incorporation efficiency, optionally at least a ten-fold improvement in ncAA incorporation efficiency, optionally at least a 20-fold improvement in ncAA incorporation efficiency, optionally at least a 30-fold improvement in ncAA incorporation efficiency, and optionally at least a 40-fold improvement in ncAA incorporation efficiency, as compared to a corresponding non-evolved and/or wild-type ribosome. Similarly, it is contemplated that certain evolved ribosomes of the instant disclosure exhibit enhanced fidelity of ncAA incorporation, as compared to the art, e.g., the evolved ribosomes of the instant disclosure exhibit at least 50% fidelity of ncAA incorporation, optionally at least 60% fidelity of ncAA incorporation, optionally at least 70% fidelity of ncAA incorporation, optionally at least 75% fidelity of ncAA incorporation, optionally at least 80% fidelity of ncAA incorporation, optionally at least 85% fidelity of ncAA incorporation, optionally at least 90% fidelity of ncAA incorporation, optionally at least 91%, 92%, 93%, 94%, 95%, 96%, 97%, 98% or at least 99% fidelity of ncAA incorporation.

PACE and rRNA-Deleted Host Strains

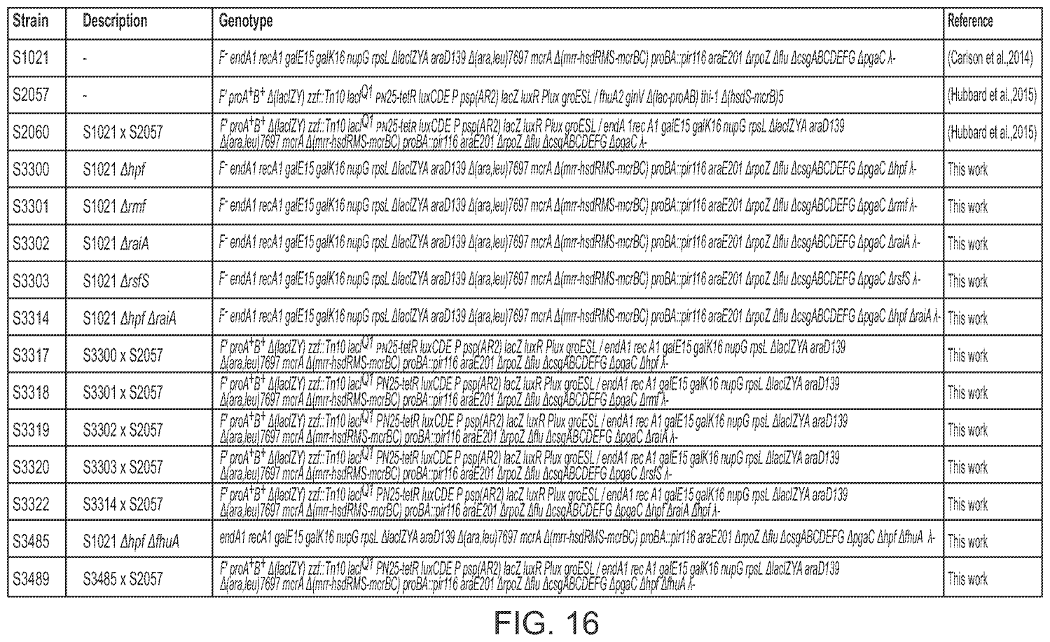

It is expressly contemplated that certain compositions and methods of the instant disclosure can employ any appropriate PACE and/or rRNA-deleted host cell. Exemplary PACE and/or rRNA-deleted host cells include, without limitation, those of FIG. 16, as well as the following:

SQ171 is an rrn E. coli strain lacking all seven chromosomal rRNA operons and carrying a single, counter-selectable plasmid bearing the wildtype rrnC operon.

KT101 is another example of a rrn⁻ E. coli strain lacking all seven chromosomal rRNA operons (rrnA, B, C, D, E, G, H). Growth of KT01 can be complemented by the rrnB operon encoded in rescue plasmid pRB101 (Kitahara et al. PNAS 109:19220-19225).

### Bacterial Culture and Transformation

Culture and transformation of bacterial cells can be performed by any art-recognized method. E. coli is commonly propagated in rich media, with examples including LB, 2× yeast extract-tryptone (YT), Terrific Broth (TB), and Super Broth (SB).

While early attempts to achieve transformation of E. coli were unsuccessful and it was at one time even believed that E. coli was refractory to transformation, Mandel and Higa (J. Mol. Bio. 53:159-162 (1970)) found that treatment with CaCl₂) allowed E. coli bacteria to take up DNA from bacteriophage λ. In 1972, Cohen et al. showed CaCl₂-treated E. coli bacteria were effective recipients for plasmid DNA (Cohen et al., Proc. Natl. Acad. Sci., 69:2110-2114 (1972)). Since transformation of E. coli is an essential step or cornerstone in many cloning experiments, it is desirable that it be as efficient as possible (Lui and Rashidbaigi, BioTechniques 8:21-25 (1990)). Hanahan (J. Mol. Biol. 166:557-580 (1983), herein incorporated by reference) examined factors that affect the efficiency of transformation, and devised a set of conditions for optimal efficiency (expressed as transformants per μg of DNA added) applicable to most E. coli K12 strains. Typically, efficiencies of 10⁷ to 10⁹ transformants/μg can be achieved depending on the strain of E. coli and the method used (Liu & Rashidbaigi, BioTechniques 8:21-25 (1990), herein incorporated by reference).

Many methods for bacterial transformation are based on the observations of Mandel and Higa (J. Mol. Bio. 53:159-162 (1970)). Apparently, Mandel and Higa's treatment induces a transient state of “competence” in the recipient bacteria, during which they are able to take up DNAs derived from a variety of sources. Many variations of this basic technique have since been described, often directed toward optimizing the efficiency of transformation of different bacterial strains by plasmids. Bacteria treated according to the original protocol of Mandel and Higa yield 10⁵-10⁶ transformed colonies/μg of supercoiled plasmid DNA. This efficiency can be enhanced 100- to 1000-fold by using improved strains of E. coli (Kushner, In: Genetic Engineering: Proceedings of the International Symposium on Genetic Engineering, Elsevier, Amsterdam, pp. 17-23 (1978): Norgard et al., Gene 3:279-292 (1978); Hanahan, J. Mol. Biol. 166:557-580 (1983)) combinations of divalent cations ((Kushner, In: Genetic Engineering: Proceedings of the International Symposium on Genetic Engineering, Elsevier, Amsterdam, pp. 17-23 (1978)) for longer periods of time (Dagert and Ehrlich, Gene 6:23-28 (1979)) and treating the bacteria with DMSO (Kushner, In: Genetic Engineering: Proceedings of the International Symposium on Genetic Engineering, Elsevier, Amsterdam, pp. 17-23 (1978)), reducing agents, and hexamminecobalt chloride (Hanahan (J. Mol. Biol. 166:557-580 (1983).

A number of procedures exist for the preparation of competent bacteria and the introduction of DNA into those bacteria. A very simple, moderately efficient transformation procedure for use with E. coli involves re-suspending log-phase bacteria in ice-cold 50 mM calcium chloride at about 10¹⁰ bacteria/ml and keeping them ice-cold for about 30 min. Plasmid DNA (0.1 mg) is then added to a small aliquot (0.2 ml) of these now competent bacteria, and the incubation on ice continued for a further 30 min, followed by a heat shock of 2 min at 42° C. The bacteria are then usually transferred to nutrient medium and incubated for some time (30 min to 1 hour) to allow phenotypic properties conferred by the plasmid to be expressed, e.g. antibiotic resistance commonly used as a selectable marker for plasmid-containing cells. Protocols for the production of high efficiency competent bacteria have also been described and many of those protocols are based on the protocols described by Hanahan (J. Mol. Biol. 166:557-580 (1983).

Another rapid and simple method for introducing genetic material into bacteria is electoporation (Potter, Anal. Biochem. 174:361-73 (1988)). This technique is based upon the original observation by Zimmerman et al., J. Membr. Biol. 67:165-82 (1983), that high-voltage electric pulses can induce cell plasma membranes to fuse. Subsequently, it was found that when subjected to electric shock (typically a brief exposure to a voltage gradient of 4000-16000 V/cm), the bacteria take up exogenous DNA from the suspending solution, apparently through holes momentarily created in the plasma membrane. A proportion of these bacteria become stably transformed and can be selected if a suitable marker gene is carried on the transforming DNA transformed (Newman et al., Mol. Gen. Genetics 197:195-204 (1982)). With E. coli, electroporation has been found to give plasmid transformation efficiencies of 10⁹-10¹⁰/μg DNA (Dower et al., Nucleic Acids Res. 16:6127-6145 (1988)).

Bacterial cells are also susceptible to transformation by liposomes (Old and Primrose, In Principles of Gene Manipulation: An Introduction to Gene Manipulation, Blackwell Science (1995)). A simple transformation system has been developed which makes use of liposomes prepared from cationic lipid (Old and Primrose, (1995)). Small unilamellar (single bilayer) vesicles are produced. DNA in solution spontaneously and efficiently complexes with these liposomes (in contrast to previously employed liposome encapsidation procedures involving non-ionic lipids). The positively-charged liposomes not only complex with DNA, but also bind to bacteria and are efficient in transforming them, probably by fusion with the cells. The use of liposomes as a transformation or transfection system is called lipofection.

B. subtilis (as well as other microbes) can be grown in culture via art-recognized methods. Transformation of B. subtilis can be achieved via methods including electroporation, protoplast transformation (B. subtilis protoplasts can be transformed but are fragile, with only about 1-10% of protoplasts surviving transformation and becoming regenerated) and use of natural competence, among other methods (see, e.g., Zhang and Zhang. Microb. Biotechnol. 4:98-105).

### Pathogenic Microbes

Contemplated pathogenic microbes of the instant disclosure include, without limitation, bacteria from the following genera: Bordetella, Borrelia, Brucilla, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio and Yersinia.

In certain embodiments, the pathogenic microbe is a bacteria or bacterial combination selected from among the following: Mycobacterium tuberculosis, Bifidobacterium longum, Veillonella parvula, Clostridium difficile, Bacillus subtilis, Escherichia coli, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica, Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris, Haemophilus influenza and Shigella spp. (e.g., Shigella flexneri, Shigella dysenteriae, Shigella sonnei, Shigella boydii).

### Commensal Microbes

Contemplated commensal microbes of the instant disclosure include, without limitation, microbes of the phyla Firmicutes, Bacteroidetes, Bifidobacteria, Eubacteria, Ruminococcus, Lactobacillus, Peptococcus, Proteobacteria, Verrumicrobia, Actinobacteria, Fusobacteria and/or Cyanobacteria, as well as combinations thereof.

### Kits

The instant disclosure also provides kits containing agents of this disclosure for use in the methods of the present disclosure. Kits of the instant disclosure may include one or more containers comprising a nucleic acid construct, organism, or other component of the system(s) described herein.

The instructions generally include information as to use of the components included in the kit. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. The kits of this disclosure are in suitable packaging. Suitable packaging includes, but is not limited to, vials, bottles, jars, flexible packaging (e.g., sealed Mylar or plastic bags), and the like.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.): Ausubel et al., 1992), Current Protocols in Molecular Biology (John Wiley & Sons, including periodic updates): Glover, 1985, DNA Cloning (IRL Press, Oxford): Anand, 1992: Guthrie and Fink, 1991: Harlow and Lane, 1988, Antibodies, (Cold Spring Harbor Laboratory Press, Cold Spring Harbor, N.Y.): Jakoby and Pastan, 1979; Nucleic Acid Hybridization (B. D. Hames & S. J. Higgins eds. 1984): Transcription And Translation (B. D. Hames & S. J. Higgins eds. 1984); Culture Of Animal Cells (R. I. Freshney, Alan R. Liss, Inc., 1987): Immobilized Cells And Enzymes (IRL Press, 1986): B. Perbal, A Practical Guide To Molecular Cloning (1984): the treatise, Methods In Enzymology (Academic Press, Inc., N.Y.): Gene Transfer Vectors For Mammalian Cells (J. H. Miller and M. P. Calos eds., 1987, Cold Spring Harbor Laboratory): Methods In Enzymology, Vols. 154 and 155 (Wu et al. eds.), Immunochemical Methods In Cell And Molecular Biology (Mayer and Walker, eds., Academic Press, London, 1987): Handbook Of Experimental Immunology, Volumes I-IV (D. M. Weir and C. C. Blackwell, eds., 1986): Riott, Essential Immunology, 6th Edition, Blackwell Scientific Publications, Oxford, 1988: Hogan et al., Manipulating the Mouse Embryo, (Cold Spring Harbor Laboratory Press, Cold Spring Harbor, N.Y., 1986): Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below: All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Star Methods

Bacterial strains. All DNA manipulations were performed using NEB Turbo cells (New England Biolabs) or Mach 1F cells, which are Mach 1 T1^{R} cells (Thermo Fisher Scientific) mated with S2057 F to constitutively provide TetR and LacI. All infection assays, plaque assays and PACE experiments were performed with E. coli S3317 or S3489 as indicated. Both strains were derived from E. coli S2060 (Hubbard et al., 2015) and modified using the recombineering method (Wang and Church, 2011) as follows: (i) scarless deletion of hibernation promoting factor (HPF) (Polikanov, Blaha and Steitz, 2012) to reduce rRNA inactivation: (ii) deletion of fhuA, a lytic bacteriophage entry receptor (Killmann et al., 1995), to facilitate turbidostat PACE experiments.

DNA Cloning. Water was purified using a MilliQ water purification system (Millipore). Genes were amplified by PCR from native sources as previously described (Kolber, 2020). All plasmids and selection phages were constructed using USER cloning (Lund et al., 2014). Briefly, a single internal deoxyuracil base was included at 15-20 bases from the 5′ end of the primer. This region is described as the USER junction, which specifies the homology required for correct assembly. USER junctions were designed to contain minimal secondary structure, have 42° C.<Tₘ<70° C., and begin with a deoxyadenosine and end with a deoxythymine (to be replaced by deoxyuridine). Phusion U Hot Start DNA Polymerase (Life Technologies) was used in primers carrying deoxyuracil bases. MinElute PCR Purification Kit (Qiagen) was used to purify all PCR products to 10 μl final volume, which was quantified using a NanoDrop 1000 Spectrophotometer (Thermo Fisher Scientific). For USER assembly, an equimolar ratio (up to 1 pmol each) of PCR products carrying complementary USER junctions were mixed in a 10 μl reaction containing 0.75 units DpnI (New England Biolabs), 0.75 units USER (Uracil-Specific Excision Reagent: Endonuclease VIII and Uracil-DNA Glycosylase) enzyme (New England Biolabs), 1 unit of CutSmart Buffer (50 mM potassium acetate, 20 mM Tris-acetate, 10 mM magnesium acetate, 100 μg mL⁻¹ BSA at pH 7.9: New England Biolabs). The reactions were incubated at 37° C. for 20 min, followed by heating to 80° C. and slow cooling to 4° C. at 0.1° C. s⁻¹ in a thermocycler. The hybridized constructs were directly used for heat-shock transformation of chemically competent NEB Turbo E. coli cells or Mach1F E. coli cells. Agar-2×YT plates (1.8%: United States Biological) supplemented with the appropriate antibiotic(s) were used to select for transformants.

For selection phage cloning, the hybridized constructs were transformed into chemically competent S3489 cells carrying the accessory plasmid pJC175e (Carlson et al., 2014b), where pIII is produced in response to phage infection. After recovery for 12 h at 37° C. at 300 RPM in 2×YT media (United States Biological), the culture was centrifuged for 2 minutes at 10,000 RCF and the supernatant was purified using a 0.22 μm PVDF Ultrafree centrifugal filter (Millipore). The titer of each clonal phage stock was determined through plaque assays (see section below). In all cases, cloned plasmids and phages were verified by Sanger sequencing using template generated using the TempliPhi 500 Amplification Kit (GE Life Sciences) according to the manufacturer's protocol.

Plaque assays. S3317 or S3489 cells carrying the accessory plasmid of interest were grown at 37° C. to OD₆₀₀=0.6-0.9 in 2×YT (United States Biological) liquid media supplemented with the appropriate antibiotics. The stock of phage supernatant was filtered using a 0.22 μm PVDF Ultrafree centrifugal filter (Millipore Sigma) and diluted in three, 100-fold serial dilution increments to yield four total samples (undiluted, 102-, 104-, and 106-fold diluted). For each sample, 10 μl of phage was added to a sample library tube (VWR). S3317 or S3489 cells carrying the accessory plasmid of interest were grown at 37° C. to OD₆₀₀=0.6-0.9 in 2×YT (United States Biological) liquid media supplemented with the appropriate antibiotics. Next, 150 μl of cells were added to each library tube containing phage. Within 1-2 min of infection, 1 mL of warm (˜55° C.) top agar (0.4% agar-2×YT) supplemented with 0.04% Bluo-Gal (Gold Biotechnology) was added to the phage/cell mixture. After mixing by pipetting up and down once, each 1.16-mL mixture was plated onto one quadrant of a quartered plate with 2 mL of bottom agar (1.8% agar-2×YT). After solidification of the top agar, the plates were grown overnight (˜18 h) at 37° C. before plaques, stained blue following Bluo-Gal cleavage, could be observed.

Enrichment assays. S3317 or S3489 cells carrying the accessory plasmid of interest were grown in Davis rich media (DRM) media (Carlson et al., 2014b) supplemented with the appropriate antibiotics to OD₆₀₀=0.2. The SP supernatant was added to a final titer of 105 pfu mL⁻¹ and grown for 14-18 h in a 37° C. shaker at 300 RPM. Cultures were centrifuged using a table-top centrifuge for 2 min (10,000 RCF). The supernatant was filtered through a 0.22 μm PVDF Ultrafree centrifugal filter (Millipore Sigma) and titered by plaque assay on S3317 or S3489 cells with pJC175e (total phage titer), S3317 or S3489 cells with ^{proC}AP3_{H3} (activity dependent phage titer, pAB171c), and/or S3317 or S3489 cells without any AP (recombinant M13-like SP titer). If necessary, purified phage samples were stored overnight at 4° C. prior to plaquing.

PACE. Host cell cultures, lagoons, media, and the PACE apparatus were prepared as previously described (Esvelt, Carlson and Liu, 2011). Briefly, MP6 (Badran and Liu, 2015a) was co-transformed into chemically competent S3489 or S3317 cells alongside the AP of interest and recovered for 45 min at 37° C. using DRM supplemented with 25 mM D-fucose to ensure MP repression via catabolite repression by glucose (a component of DRM) and competitive inhibition of araC by D-fucose (Wilcox, 1974: Soisson et al., 1997). Transformations were selected on 1.8% agar-2×YT plates containing kanamycin (30 μg mL⁻¹), chloramphenicol (40 μg mL⁻¹), 25 mM glucose (United States Biological), and 25 mM D-fucose (Carbosynth). After incubation at 37° C. for 12-18 h, six individual colonies were picked, resuspended in DRM, 10-fold serially diluted and plated on 1.8% agar-2×YT plates with kanamycin (30 μg mL⁻¹), chloramphenicol (40 μg mL⁻¹) and containing either 25 mM arabinose (Gold Biotechnology) or 25 mM glucose and 25 mM D-fucose. After incubation for 12-18 h at 37° C., the plates were examined to confirm arabinose sensitivity. Concomitant with the aforementioned plating step, the resuspended colonies and dilutions thereof were used to inoculate liquid cultures in DR supplemented with kanamycin (30 μg mL⁻¹), chloramphenicol (40 μg mL⁻¹), 25 mM glucose, and 25 mM D-fucose. The cultures were grown in a 37° C. shaker at 900 RPM (Infors HT Multitron Pro) to OD₆₀₀=0.2, at which point 1-mL of cells were added directly to 300 mL of fresh DRM in the turbidostat. The turbidostat culture was maintained at 300 mL and optical density was maintained at OD₆₀₀=0.8-0.9 using a TruCell2 probe (Esvelt, Carlson and Liu, 2011). All lagoons supplied by the turbidostat were maintained at 40 mL, and diluted as described previously (Esvelt, Carlson and Liu, 2011). Prior to infection with SPs, lagoons were supplemented to a final concentration of 25 mM arabinose using a syringe pump (New Era Pump Systems) for 1 h to induce the MP. At the indicated time points, samples were collected from each lagoon and SP was purified as described above.

Mutagenesis during PACE. The basal mutation rate of replicating filamentous phage in E. coli is 7.2×10⁻⁷ substitutions per base pair per generation, which is sufficient to generate all possible single but not double mutants of a given 1,000 base pair gene in a 40-mL lagoon after one generation of phage replication. For the 16S ribosomal subunit (1,542 base pairs), a basal mutation rate of 7.2×10⁻⁷ substitutions per base pair per generation applied to 2×1010 copies of the gene (a single generation in a 40-mL lagoon) yields ˜2.2×10⁷ base substitutions (7.2×10⁻⁷ substitutions per base pair*1,542 base pairs*2×1010 copies), which could cover all 4.6×10³ single point mutants and all ˜2.1×10⁷ double point mutants. Arabinose induction of the high-potency mutagenesis plasmid MP6 (Badran and Liu, 2015a) increases the phage mutation rate to 7.2×10⁻³ substitutions per base pair per generation, yielding ˜2.2×10¹¹ substitutions spread over 2×1010 copies of the gene after a single generation. This elevated mutation rate is sufficient to cover all possible single mutants (4.6×10³ possibilities), double mutants (2.1×10⁷ possibilities), and triple mutants (9.9×10¹⁰ possibilities) after a single phage generation.

Luminescence assays. Log-phase (OD₆₀₀=0.3-0.5) S3489 cells carrying the reporter plasmid (RP) pFL19c grown in 2×YT (United States Biological) were made chemically competent, later transformed with the desired EP, and recovered for 2 h in Terrific Broth (Millipore Sigma). All transformations were plated on 1.8% agar-2×YT plates (United States Biological) supplemented with kanamycin (30 μg mL⁻¹) and carbenicillin (50 μg mL⁻¹). The plates were incubated for 12-18 h in a 37° C. incubator. Colonies transformed with the appropriate EP were picked the following day and grown in DRM containing kanamycin (30 μg mL⁻¹) and carbenicillin (50 μg mL⁻¹) for 18 h. Following overnight growth of the EP/RP-carrying strains, cultures were diluted 100-fold into fresh DRM supplemented with kanamycin (30 μg mL⁻¹) and carbenicillin (50 μg mL⁻¹). The cultures were induced with anhydrotetracycline (1000 ng mL⁻¹), and 200 μL of each culture was transferred to a 96-well black wall, clear bottom plate (Costar®) and topped with 20 μL of mineral oil (Millipore Sigma®). OD600 and luminescence values for each well was monitored using an Infinite M1000 Pro microplate reader (Tecan®) over 15 h. Each variant was assayed in 8-24 biological replicates. Luminescence activities were tabulated at OD₆₀₀=0.15 in all cases.

Fluorescent protein assays. Chemically competent 3489 were transformed with ribosome expression plasmid (EP) and desired reporter plasmid (RP), and recovered for 2 h in Terrific Broth (Millipore Sigma®). All transformations were plated on 1.8% agar-2×YT plates (United States Biological) supplemented with kanamycin (30 μg mL⁻¹) and carbenicillin (50 μg mL⁻¹). The plates were incubated for 12-18 h in a 37° C. incubator. Colonies transformed with the appropriate EP were picked the following day and grown in DRM containing kanamycin (30 μg mL⁻¹), carbenicillin (50 μg mL⁻¹), and anhydrotetracycline (1000 ng mL⁻¹). After growth for 16-24 hr at 37° C. with 900 rpm shaking, 200 μL of each culture was transferred to a 96-well black wall, clear bottom plate (Costar®) and topped with 20 μL of mineral oil (Millipore Sigma®). OD₆₀₀ and fluorescence values (excitation at 485 nm, emission at 510 nm) for each well were monitored using an Infinite M1000 Pro microplate reader (Tecan®) or Spark plate reader (Tecan®). Each variant was assayed in 4-8 biological replicates. Fluorescent protein yields were normalized to culture OD₆₀₀ in all cases.

ncAA incorporation assays. Chemically competent 3489 were transformed with complementary plasmid (CP) pTECH Mb PyIRS IPYE (Bryson et al., 2017) with resistance changed for DHFR, and desired EP and RP (pAB140g (WT sfGFP) or pAMC025a (UAG151 sfGFP) or pAMC016a (WT luxAB) or pAMC016b (UAG luxAB)), and recovered for 2 h in Terrific Broth (Millipore Sigma®). All transformations were plated on 1.8% agar-2×YT plates (United States Biological) supplemented with trimethoprim (3 μg mL⁻¹), kanamycin (10 μg mL⁻¹), and carbenicillin (15 μg mL⁻¹). The plates were incubated for 12-18 h in a 37° C. incubator. Colonies transformed with the appropriate EP and RP were picked the following day and grown in DRM containing trimethoprim (3 μg mL), kanamycin (10 μg mL⁻¹), and carbenicillin (15 μg mL⁻¹) for 20-24 h. Overnight cultures were 100-fold for luminescence assay's in fresh DRM containing: (3 μg mL), kanamycin (10 μg mL⁻¹), carbenicillin (15 μg mL⁻¹), anhydrotetracycline (40 ng mL⁻¹), with or without N_{ε}-((tertbutoxy)carbonyl)-L-lysine (Bock) (1 mM) (Bachem). For sfGFP assays colonies were picked directly into complete assay media. OD₆₀₀ and luminescence values for each assay monitored using an Infinite M1000 Pro microplate reader (Tecan®) or Spark plate reader (Tecan®). Each variant was assayed in 4-8 biological replicates. Luminescence activities were tabulated at OD₆₀₀=0.15 in all cases. Fluorescent protein yield was normalized to culture OD₆₀₀ at saturation (OD₆₀₀˜1.5).

SQ171 complementation assays. Log-phase (OD₆₀₀=0.3-0.5) cells of SQ171 (Asai et al. 1999a: Asai et al. 1999b) grown in 2×YT (United States Biological) were transformed with the desired EP, and recovered for 5 h in 2×YT in a 37° C. shaker. The recovery culture was centrifuged at 10,000 RCF for 2 min, then the pellet was resuspended in 100 μL 2×YT. The resuspended cells were diluted serially in seven, 10-fold increments to yield eight total samples (undiluted, 10¹-, 10²-, 10³-, 10⁴-, 10⁵-, 10⁶-, and 10⁷-fold diluted). To determine the efficiencies of EP transformation and counter-selectable plasmid curing, 3 μl of each sample of the diluted series were plated on 1.8% agar-2×YT plates (United States Biological) supplemented with spectinomycin (100 μg mL⁻¹) and carbenicillin (50 μg mL⁻¹), with or without 5% sucrose (Millipore Sigma). For picking single colonies, the remaining undiluted cells were plated on 1.8% agar-2×YT plates (United States Biological) containing spectinomycin (100 μg mL⁻¹), carbenicillin (50 μg mL⁻¹) and 5% sucrose. All plates were grown for 12-18 h in a 37° C. incubator. Colonies transformed with the appropriate EP and surviving sucrose selection were picked and grown in DRM containing spectinomycin (100 μg mL⁻¹), carbenicillin (50 μg mL⁻¹), and 5% sucrose. Following overnight growth of the EP-carrying strains, cultures were diluted 250-fold into fresh DRM containing spectinomycin (100 μg mL⁻¹) and carbenicillin (50 μg mL⁻¹). From the diluted cultures, 150 μl of each culture was transferred to a 96-well black wall, clear bottom plate (Costar), topped with 20 μL of mineral oil, and the OD₆₀₀ was measured every 5 min over 15 h. Separately, 400 μL of each diluted culture was supplemented with kanamycin (30 μg mL⁻¹) and grown in a 37° C. shaker at 300 RPM. Colonies that survived selection in kanamycin were excluded from final analysis, as survival in kanamycin indicates persistence of the resident pCSacB plasmid (which carries a KanR resistance cassette). The doubling time of each culture was calculated using the Growthcurver package (version 0.3.0) (Sprouffske, Jul. 30, 2018) in R (version 3.5.2).

Cell volume measurement. Complemented SQ171 strains were grown overnight 16-18 h in a 37° C. incubator DRM containing spectinomycin (100 μg mL⁻¹) and carbenicillin (50 μg mL⁻¹). Overnight cultures were then diluted 100-fold in fresh DRM containing spectinomycin (100 μg mL⁻¹) and carbenicillin (50 μg mL⁻¹). Upon reaching early log-phase (OD₆₀₀=0.1-0.15), cells were diluted 10-fold to synchronize cultures and harvested when early log-phase was reached again (OD₆₀₀=0.1-0.15). Cells were then placed on ice and cell volumes were measured in filtered PBS (0.2 μm filter) using Coulter Counter (Beckman Coulter) with a 20 μm aperture. Particles smaller than 0.4 μm³ in volume were excluded from analysis. Measurements were calibrated using NIST traceable 3.0 μm diameter polystyrene beads (ThermoFisher).

Cell viability and AHA incorporation assays. SQ171 strains were grown for 24 h in M9 minimal media supplemented with all amino acids (defined as M9AA): M9 salts (Teknova), [0.4% w/v] D-glucose, [3.4 mg/mL] thiamine hydrochloride, [1 mM] MgSO₄, [0.25 mM] CaCl₂, [1.33 mg/L] amino acid mix (-Methionine) (MANU), 200 μM L-Methionine (Sigma Aldrich), spectinomycin (100 μg mL⁻¹) and carbenicillin (50 μg mL⁻¹). Overnight cultures were diluted 100-fold in fresh M9AA and grown to OD₆₀₀=0.1-0.15. Cultures were synchronized by diluting once more by 10-fold and continuing to grow: At OD₆₀₀=0.1-0.15, cultures were harvested by centrifugation at 3000 RCF for 5 min, media was exchanged for M9AA-M (defined as M9AA excluding L-Methionine), and cultures returned to 37° C. incubator at 300 RPM. After 1 h, M9AAM outgrowth cells were treated with 200 μM L-azidohomoalanine (AHA) (Click Chemistry Tools) and BacLight RedoxSensor Green Vitality Kit reagents (Invitrogen) per the manufacturer protocols for 5, 10, 15, 20 or 30 min at 37° C. and 300 RPM. AHA incorporation was blocked at each time interval by adding 200 μg mL chlorenphenicol, whereas RedoxSensor Green was 23 blocked at each time interval by adding 10 mM NaN₃. Negative control 684 samples for AHA incorporation and cell vitality were treated with 200 μg mL chlorenphenicol or 10 mM NaN₃, respectively, 10 minutes prior to AHA or RedoxGreen addition. Following AHA incorporation and vitality labelling, cells were washed using 0.5 mL PBS, fixed in 3.8% PFA for 10 min at room temperature, washed twice with PBS, permeabilized with 0.2% Triton X00 for 10 min in RT, and washed twice more in PBS. Samples were stored at 4° C. for subsequent Click-IT chemistry. Fixed and permeabilized cell samples were mixed with Click-&-Go Cell Reaction Buffer (Click Chemistry Tools) containing 2.5 μM AlexaFluor 405 Alkyne (Click Chemistry Tools) according to manufacturer instructions, and were incubated for 30 min in the dark at room temperature, then washed twice with PBS. Labelled cells were analyzed with BD Biosciences flow cytometer LSR II HTS with excitation lasers at 405, 488 and 561 nm and emission filters at 450/50, 515/20 and 610/20 nm. Cells were gated on forward and side scatter, and particles/cells with minimal vitality labeling were excluded. The AHA incorporation rate represents the rate of linear increase in population mean AHA incorporation over 20 mins. For viability assays not investigating AHA incorporation, strains were grown in DRM. Overnight cultures were diluted 100-fold in fresh M9AA and grown to OD₆₀₀=0.1-0.15. Cultures were again synchronized by diluting once more by 10-fold and continuing to grow. At OD₆₀₀=0.1-0.15, cultures were labeled with BacLight RedoxSensor Green Vitality Kit reagents (Invitrogen) per the manufacturer protocols, for 30 min at 37° C. with 300 rpm shaking.

Aminoglycoside sensitivity assays. SQ171 strains carrying wild-type or evolved rRNA variants were grown in DRM containing spectinomycin (100 μg mL⁻¹) and carbenicillin (50 μg mL⁻¹) for 128 h. Overnight cultures were diluted 50-fold in fresh DRM containing spectinomycin (100 μg mL⁻¹), carbenicillin (50 μg mL⁻¹) and mixed 1:1 with a dilution series of kanamycin or gentamicin (64, 32, 16, 8, 4, 2, 1, 0.5, 0.25 μg mL⁻¹). Cultures were grown at 37° C. with shaking, 900 rpm, overnight for 24 h. OD₆₀₀ for each well was quantified using an Infinite M1000 Pro microplate reader (Tecan). IC₅₀ values for kanamycin and gentamicin resistance of each strain were calculated in Prism (v 9.1.0).

Protein purification. SQ171 strains transformed with pED17xl (sfGFP with C-terminal His-tag) were lysed by B-per (Thermo Fisher), 4 mL per gram weight of pellet. To each sample 120 μL of B-per+protease inhibitor (Roche) was added and incubated for 1 hr at room temperature with gentle rocking. Soluble protein was fractionated by centrifugation at 16,000×g for 20 mins and removing supernatant (soluble protein). 300 μL of each sample was loaded onto a His-Spin 24 Protein Mini-prep column (Zymo) and purified using manufacturer's protocol. All samples were eluted in 150 μL of elution buffer. Gel-code blue stained SDS-PAGE gel lanes were subdivided into 7 regions and cut into ˜2 mm squares. These were washed overnight in 50% methanol/water. These were washed once more with 1:1 methanol: water for overnight, dehydrated with acetonitrile and dried in a speed-vac. Reduction and alkylation of disulfide bonds was then carried out by the addition of 30 μl 10 mM dithiothreitol (DTT) in 100 mM ammonium bicarbonate for 30 minutes to reduce disulfide bonds. The resulting free cysteine residues were subjected to an alkylation reaction by removal of the DTT solution and the addition of 100 mM iodoacetamide in 100 mM ammonium bicarbonate for 30 minutes to form carbamidomethyl cysteine. These were then sequentially washed with aliquots of acetonitrile, 100 mM ammonium bicarbonate and acetonitrile and dried in a speed-vac. The bands were enzymatically digested by the addition of 300 ng of trypsin (or chymotrypsin for R or K qtRNAs) in 50 mM ammonium bicarbonate to the dried gel pieces for 10 minutes on ice. Depending on 22 the volume of acrylamide, excess ammonium bicarbonate was removed or enough was added to rehydrate the gel pieces. These were allowed to digest overnight at 37° C. with gentle shaking. The resulting peptides were extracted by the addition of 50 μL (or more if needed to produce supernatant) of 50 mM ammonium bicarbonate with gentle shaking for 10 minutes. The supernatant from this was collected in a 0.5 ml conical autosampler vial. Two subsequent additions of 47.5/47/5/5 acetonitrile/water/formic acid with gentle shaking for 10 minutes were performed with the supernatant added to the 0.5 mL autosampler vial. Organic solvent was removed and the volumes were reduced to 15 μL using a speedvac for subsequent analyses.

Chromatographic separations and analysis. Digested extracts were analyzed by reversed phase high performance liquid chromatography (HPLC) using Waters NanoAcquity pumps and autosampler and a ThermoFisher Orbitrap Elite mass spectrometer using a nano flow configuration. A 20 mm×180 μm column packed with 5 μm Symmetry C18 material (Waters) using a flow rate of 15 μL per minute for three minutes was used to trap and wash peptides. These were then eluted onto the analytical column which was a self-packed with 3.6 μm Aeris C18 material (Phenomenex) in a fritted 20 cm×75 μm fused silica tubing pulled to a 5 μm tip. The gradient was isocratic 1% A Buffer for 1 minute 250) nL min⁻¹ with increasing B buffer concentrations to 15% B at 20.5 minutes, 27% B at 31 minutes and 40% B at 36 minutes. The column was washed with high percent B and re-equilibrated between analytical runs for a total cycle time of approximately 53 minutes. Buffer A consisted of 1% formic acid in water and buffer B consisted of 1% formic acid in acetonitrile. Mass Spectrometry: The mass spectrometer was 25 operated in a dependant data acquisition mode where the 10 most abundant peptides detected in the Orbitrap Elite (ThermoFisher) using full scan mode with a resolution of 240,000 were subjected to daughter ion fragmentation in the linear ion trap. A running list of parent ions was tabulated to an exclusion list to increase the number of peptides analyzed throughout the chromatographic run. The resulting fragmentation spectra were correlated against custom databases using PEAKS Studio X (Bioinformatics Solutions).

Calculation of Limit of Detection and relative abundance. The results were matched to a sfGFP reference and analyzed for ≤2 amino acid substitutions in a single tryptic fragment. Abundance of each residue substitution was quantified by calculating the area under the curve of the ion chromatogram for each peptide precursor. The limit of detection is 10⁴ [AU], the lower limit for area under the curve for a peptide on this instrument.

### Example 2: Development of a PACE-Compatible Orthogonal Translation System

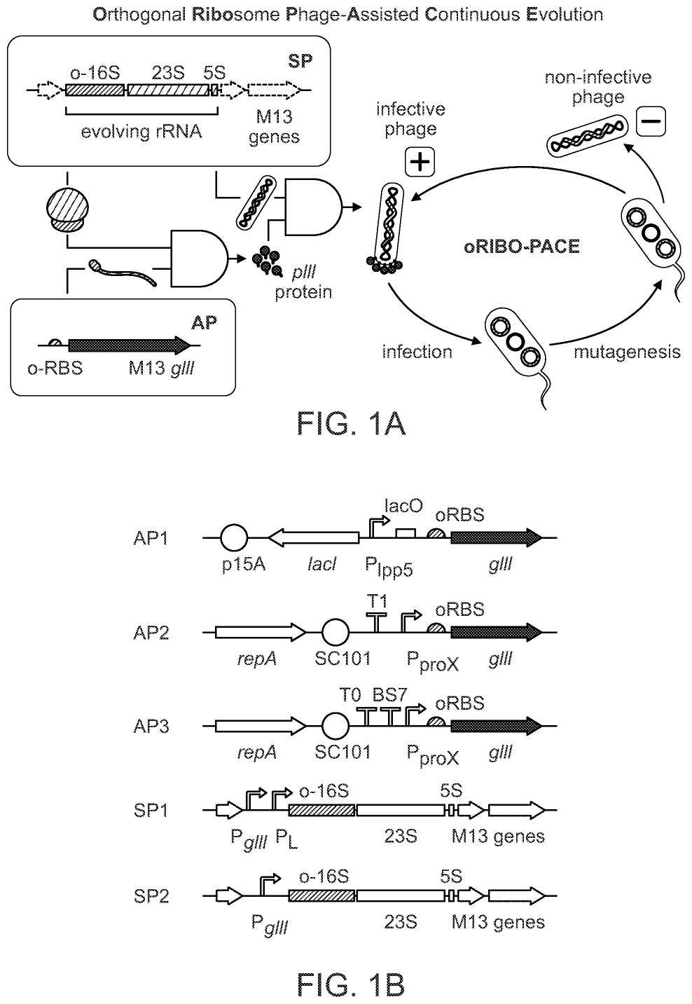

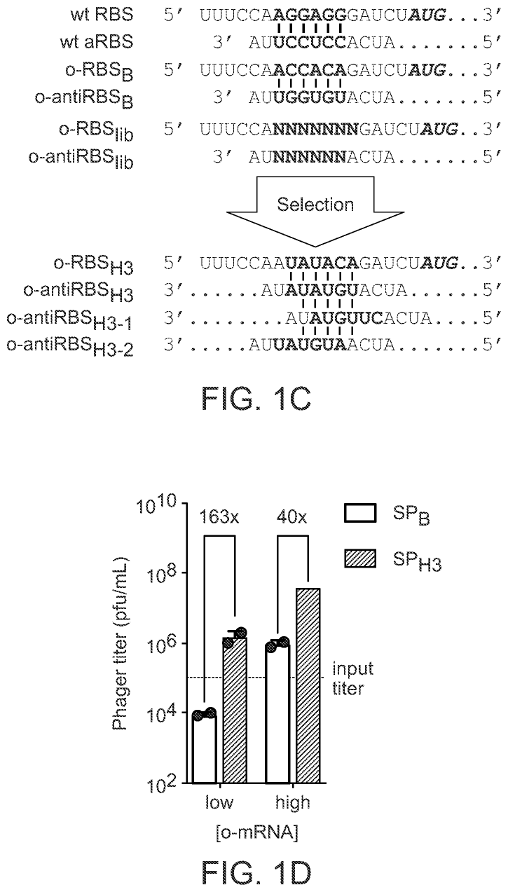

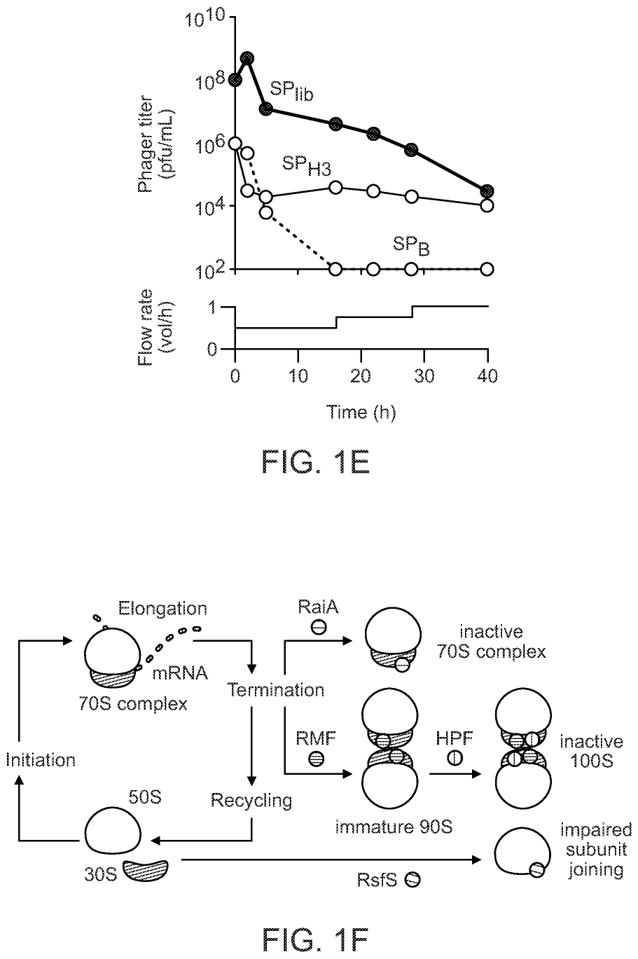

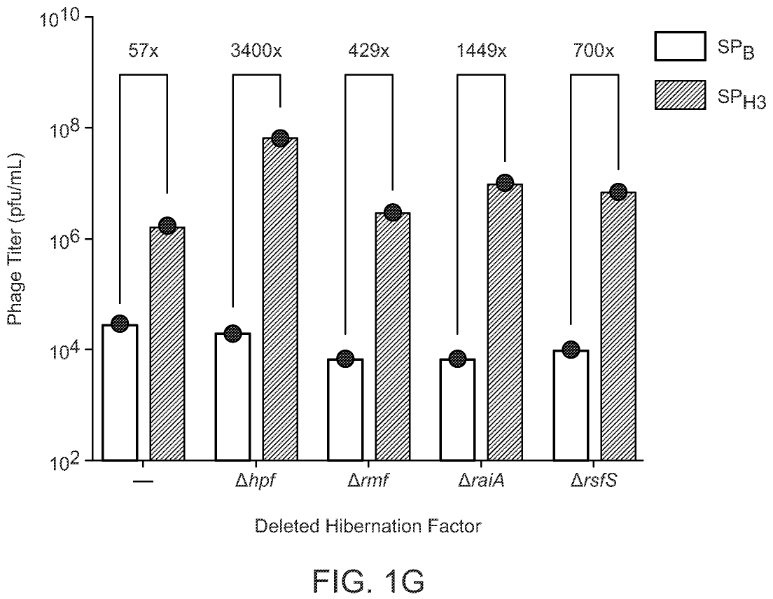

PACE has facilitated the exploration of sequence-function relationships of biomolecules with diverse cellular activities (Esvelt, Carlson and Liu, 2011: Badran et al., 2016; Badran and Liu, 2015c: Hubbard et al., 2015; Wang et al., 2018: Carlson et al., 2014b; Thuronyi et al., 2019). Briefly, PACE exploits the rapid M13 bacteriophage lifecycle and couples the production of plasmid-borne gIII, encoding the minor coat protein pIII necessary for both bacterial infection and membrane extrusion (Bennett and Rakonjac, 2006), to the activity of the evolving biomolecule encoded on a pIII-deficient phage genome. The genetic diversity of the evolving biomolecule is easily tuned through a small molecule-inducible expression of mutator proteins from the mutagenesis plasmid (MP) (Badran and Liu, 2015b). Historically PACE has been limited to protein coding genes. It was envisioned herein that PACE could be extended to the directed evolution of orthogonal rRNAs (o-rRNAs), allowing efficient traversal of mutational landscapes and uncovering variants with altered translational activity (FIG. 1A). To establish an o-rRNA PACE selection in E. coli, an orthogonal translation genetic circuit (Orelle et al., 2015: Kolber et al., 2021) was adapted to integrate the M13 bacteriophage gIII (which encodes pIII), yielding the Accessory Plasmid 1 architecture (AP1: FIG. 1B) and concurrently engineered selection phages (SPs) to encode the complementary o-rRNA operon. Functional o-rRNAs capable of forming active ribosomes and translating the gIII mRNA using the o-RBS would robustly produce pIII, yielding infectious phage progeny.

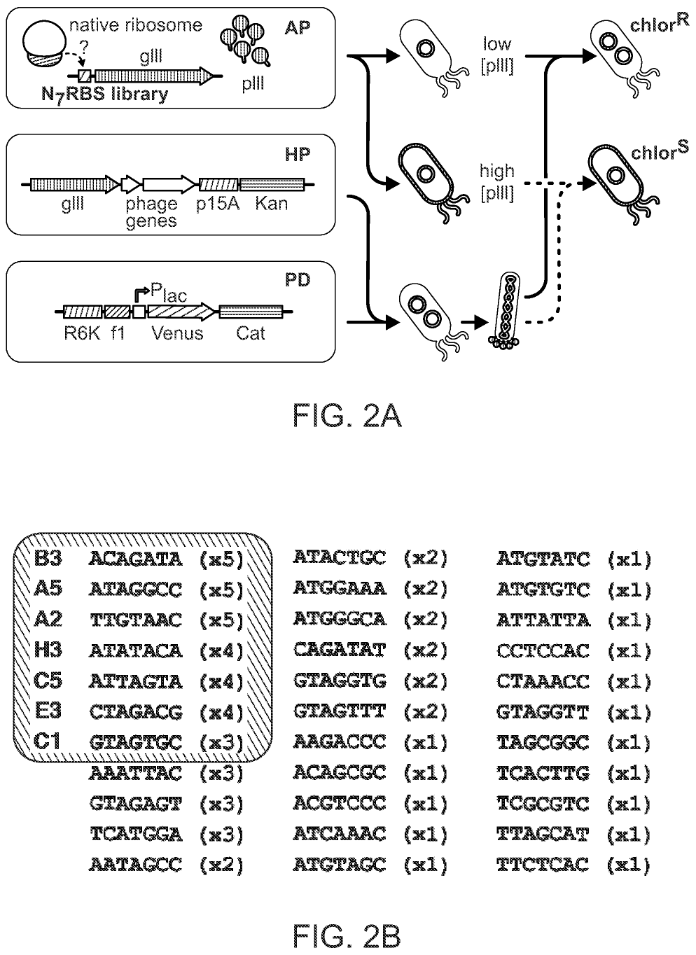

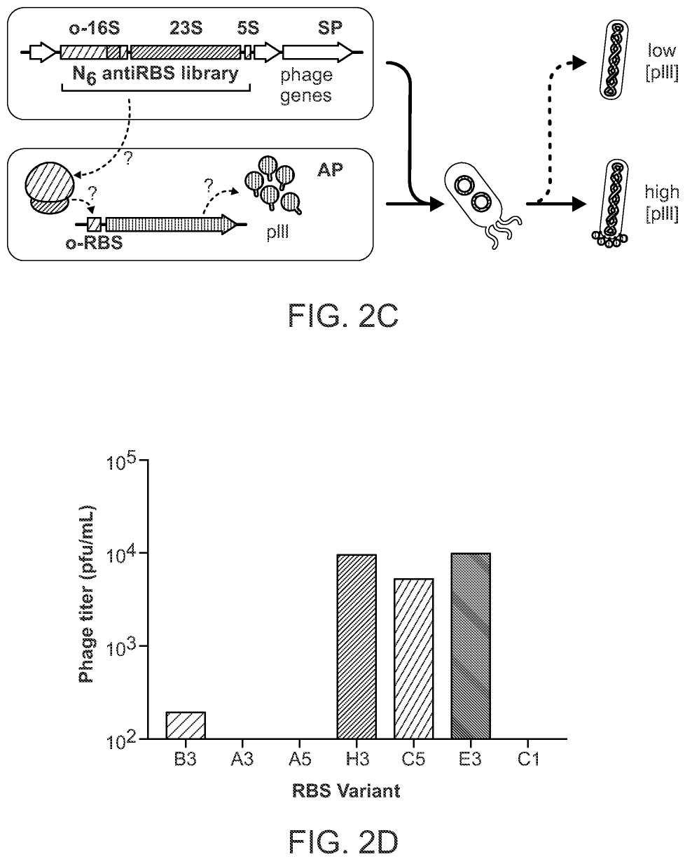

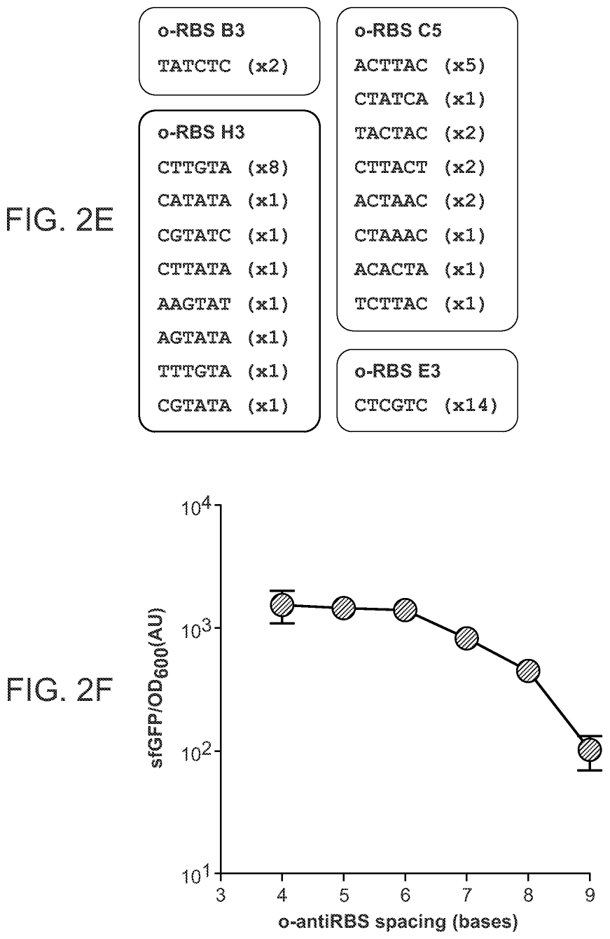

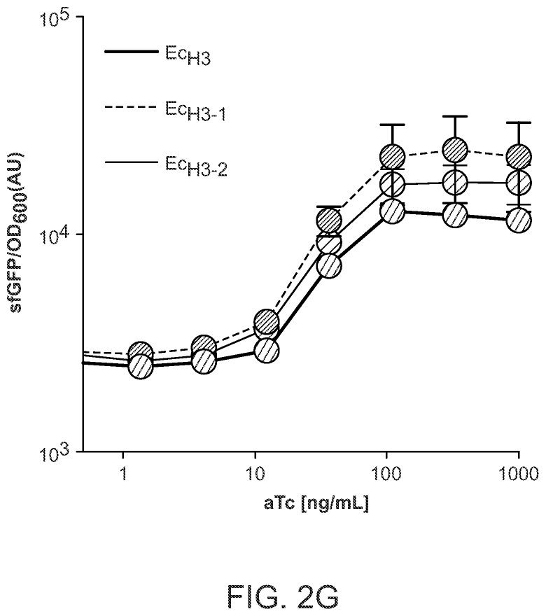

While the previously reported o-antiRBSB efficiently directs translation of an sfGFP reporter bearing the cognate o-RBSB sequence (FIG. 1C) (Rackham and Chin, 2005), direct adaptation of o-RBSB to AP1 rendered S2060 (Hubbard et al., 2015) cells uninfectable by wildtype M13 phage, indicating high background pIII expression. Thus, a two-stage selection was developed to identify PACE-compatible o-RBS/o-antiRBS pairs with reduced background translation by host ribosomes. First a degenerate library of 47 RBS variants (o-RBS_{lib}, FIG. 1C) was introduced and infectivity of the resultant cells was assessed to identify sequences poorly recognized by host ribosomes (FIG. 2A). This analysis revealed 33 putative o-RBS candidates, and the most abundant seven variants (FIG. 2B) were further characterized. To discover potential cognate o-antiRBSs, a degenerate library encoding 46 antiRBS variants in the SP-borne E. coli o-rRNA (o-antiRBS_{lib}, FIG. 1C) was introduced into E. coli host cells bearing each of the seven new o-RBSs. Functional o-antiRBS sequences should efficiently translate gIII and give rise to progeny phage (FIGS. 2C-E). After further optimization of spacer sequences (FIG. 2F), o-RBS_{H3} (FIG. 1C) was identified as an optimal orthogonal sequence for subsequent experiments. o-RBS_{H3} was adapted to AP1 (AP1_{H3}) yielding 40- to 163-fold enrichment for SPs encoding the cognate o-antiRBS_{H3} (SP_{H3}) relative to SPs bearing the mismatched o-antiRBSB sequence (SP_{B}) (FIG. 1D).

While the o-RBS_{H3}/o-antiRBS_{H3} pair enabled phage propagation in standing culture (FIG. 1D), it was hypothesized that alternative solutions may exist under continuous culture conditions. A degenerate SP library encoding 46 antiRBSs (o-antiRBS_{lib}, FIG. 1C) was continuously propagated using AP1_{H3} in S2060 cells yielding comparable phage titers to SP_{H3}, while SP_{B} was rapidly washed out (FIG. 1E). The resulting SP populations were analyzed at 40 h by Sanger sequencing (24 clones), and it was identified that SP_{lib} converged on exclusively two variants: o-antiRBS_{H3-1} and o-antiRBS_{H3-2} (FIG. 1C). Both variants robustly translated a LuxAB luciferase reporter, showing similar dynamic range to the initial o-antiRBS_{H3} variant (FIG. 2G). It was noted that o-antiRBS_{H3-1}, but not o-antiRBS_{H3-2}, appeared in the initial antiRBS library (FIG. 2E), which indicated that differential o-ribosome activities may depend on culturing conditions.

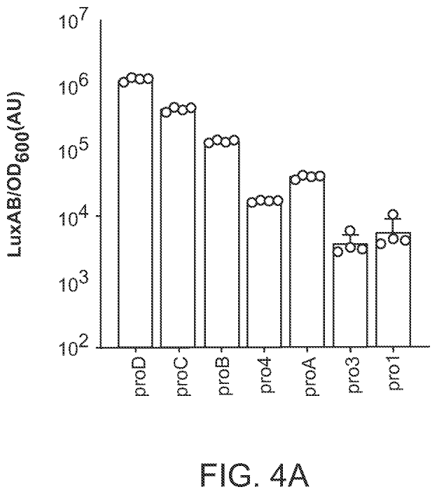

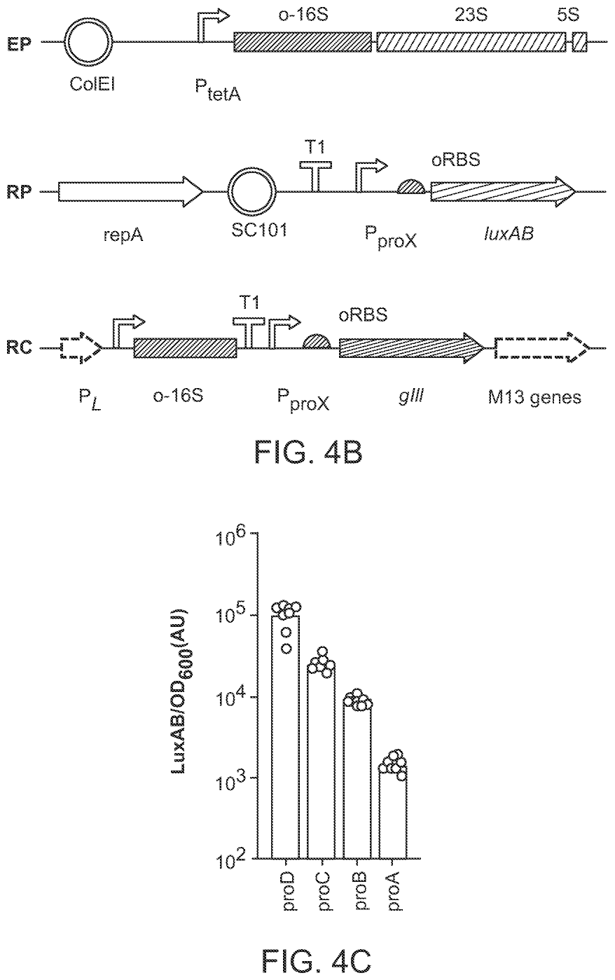

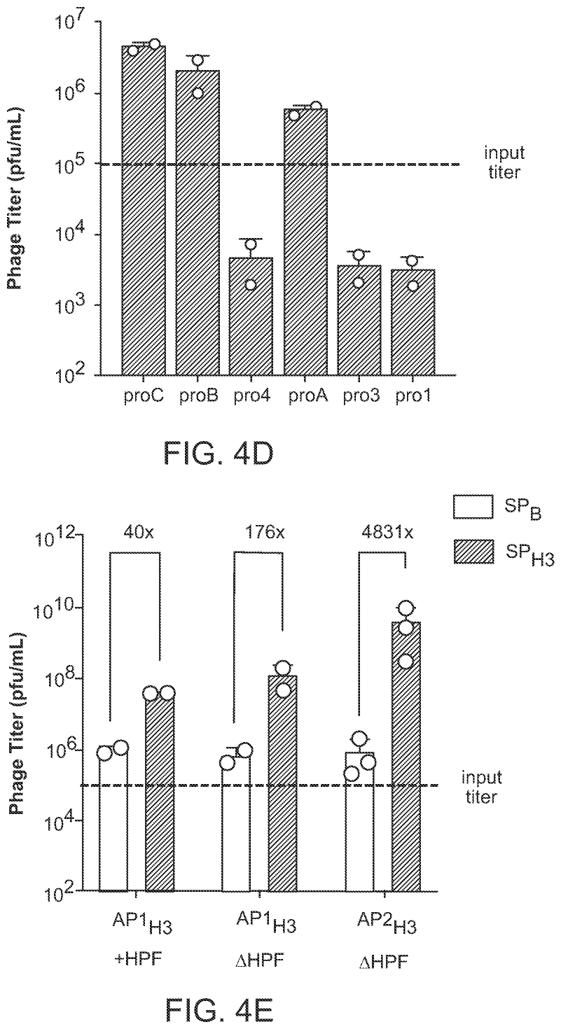

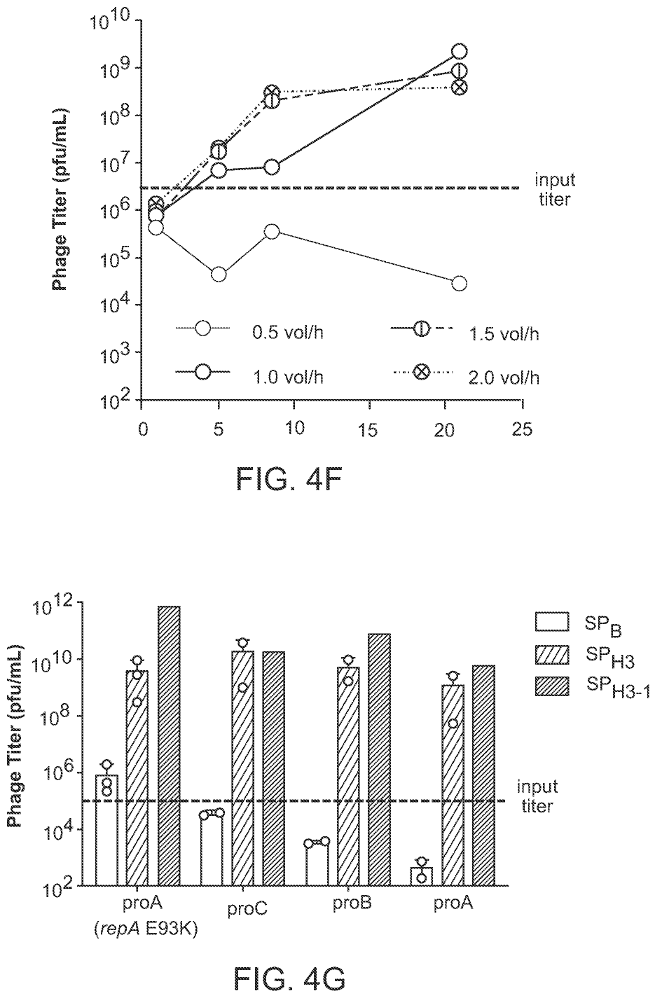

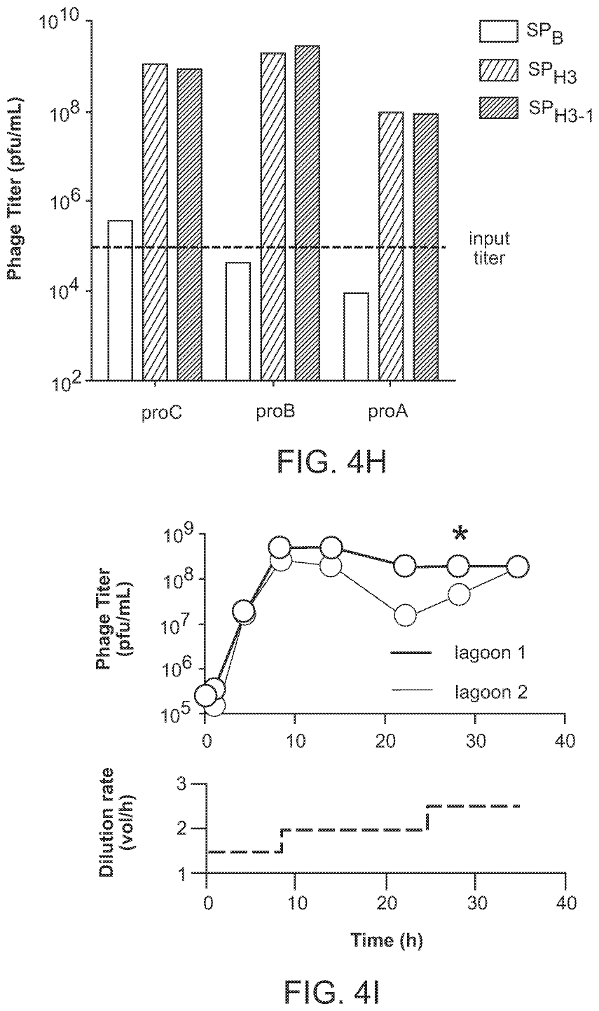

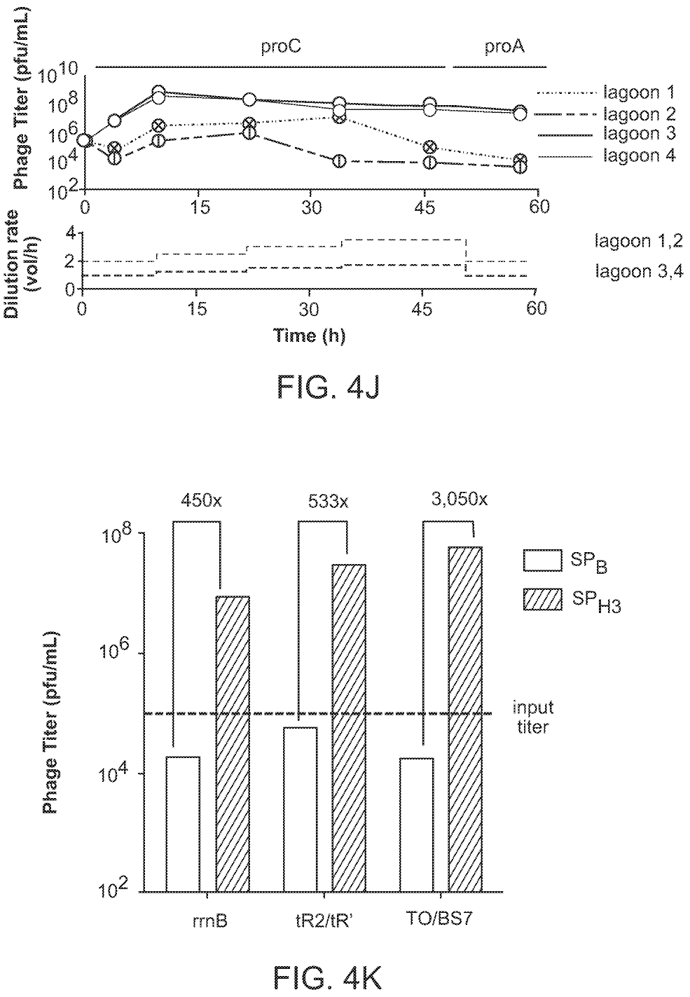

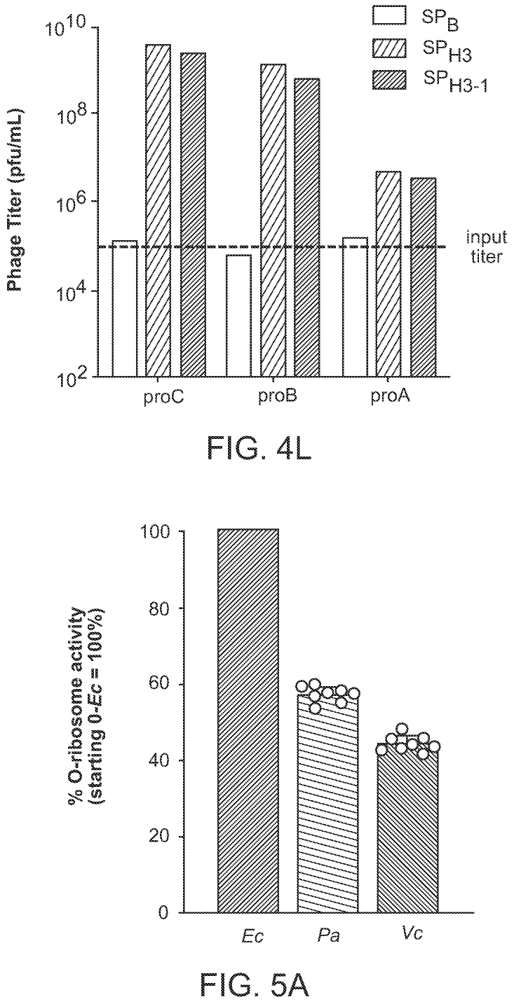

Although functional o-antiRBS sequences were successfully identified from an unbiased SP library, the final phage titers were considerably lower than those in previous protein-based PACE campaigns (Esvelt, Carlson and Liu, 2011: Badran et al., 2016; Hubbard et al., 2015; Wang et al., 2018: Carlson et al., 2014b: Thuronyi et al., 2019). It was noted that host cells in the turbidostat resided at the transition between exponential and stationary phase, during which o-rRNAs may be inactivated by hibernation factors (Polikanov, Blaha and Steitz, 2012). Accordingly, factors known to inhibit ribosome activity were deleted to improve the propagation of o-rRNA SPs (FIG. 1F). Deletion of ribosome hibernation promoting factor (HPF) from S2060 (Hubbard et al., 2015) yielded host strain S3317, which exhibited a 3,400-fold improvement in SP propagation (FIG. 1G). Concurrently, a new AP architecture was prepared, AP2 (FIG. 1B), which encoded a growth phase-independent constitutive promoter (Davis, Rubin and Sauer, 2010) to simplify o-rRNA evolution experiments (FIGS. 4A-D) and the pSC101 origin of replication was integrated for stringent copy number control (Peterson and Phillips, 2008). When introduced into S3317 cells, AP2_{H3} supported SP_{H3} propagation 4,831-fold more efficiently than the mismatched SP_{B} (FIG. 1G, FIG. 4E).

Next, all o-antiRBS SPs (FIG. 1B) were competed using S3317/AP2_{H3} under continuous flow at varying lagoon dilution rates. It was noted that low lagoon flowrates (<1.0 vol/h) led to poor SP propagation, consistent with ribosome inactivation at saturated cell densities (FIG. 4F) (Polikanov, Blaha and Steitz, 2012). Individual SPs propagated at 2 vol/h were analyzed using Sanger sequencing and found that most SPs encoded o-antiRBS_{H3-1}, in agreement with the SP_{lib} evolution experiment (FIG. 1E, FIG. 2E). In overnight enrichment assays in standing culture, SP_{H3-1} similarly showed improved titers (up to 186-fold) over SP_{H3} (FIG. 4G). Following additional strain engineering (ΔfhuA: Killmann et al., 1995) to produce S3489 (FIGS. 4G to 4H) and plasmid modification to yield the AP3 architecture (FIG. 1B) to limit AP/SP recombination (FIGS. 4B and 4I-4L), the S3489/AP3_{H3}/SP_{H3-1} combination was found to be the optimal orthogonal translation system for all subsequent experiments.

### Example 3: Continuous Directed Evolution of Orthogonal Ribosomes

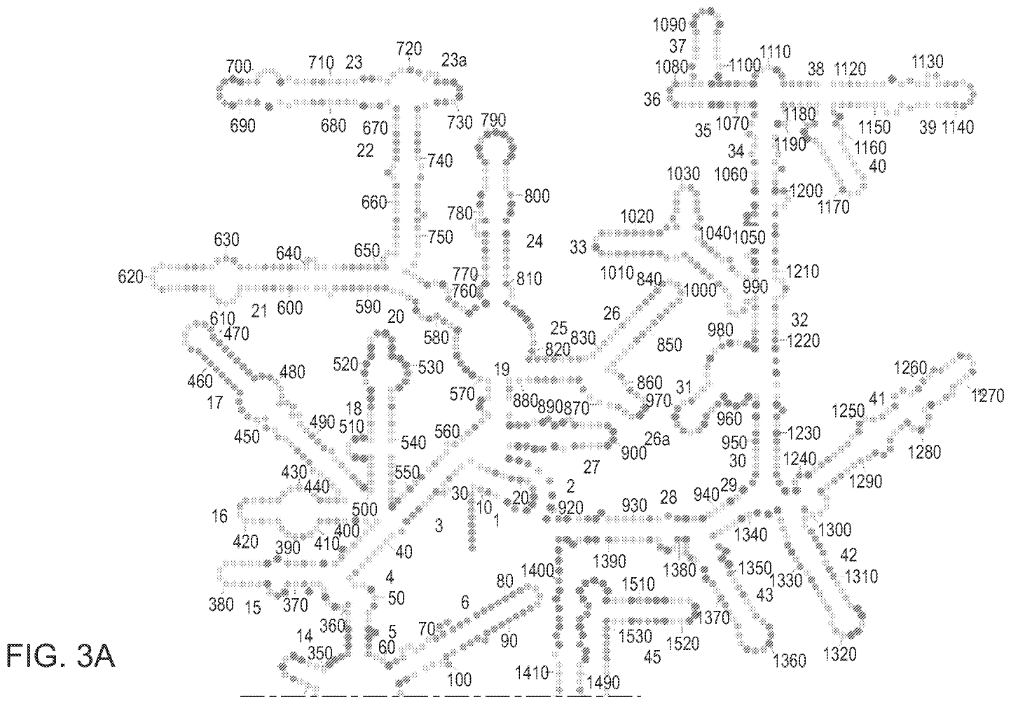

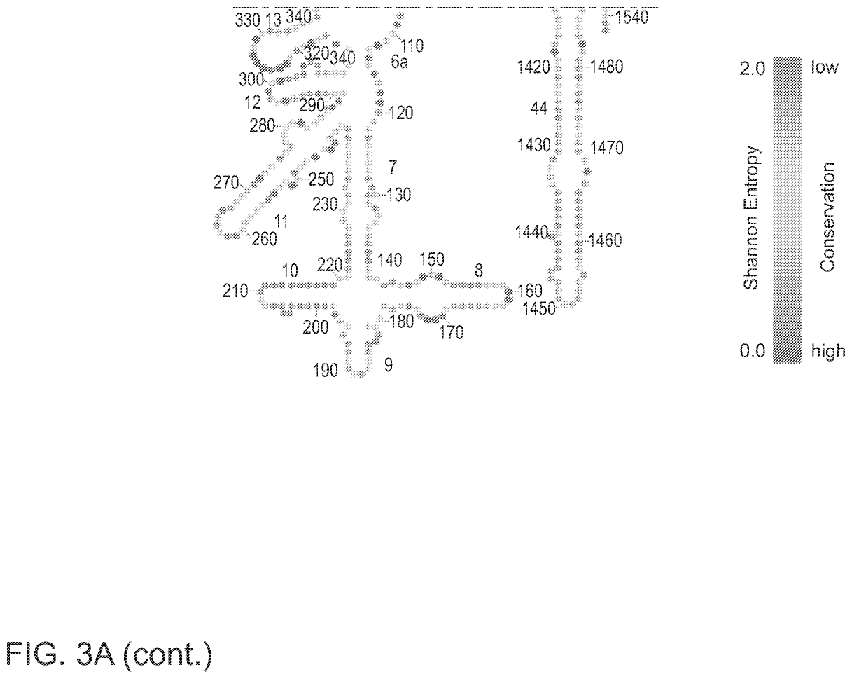

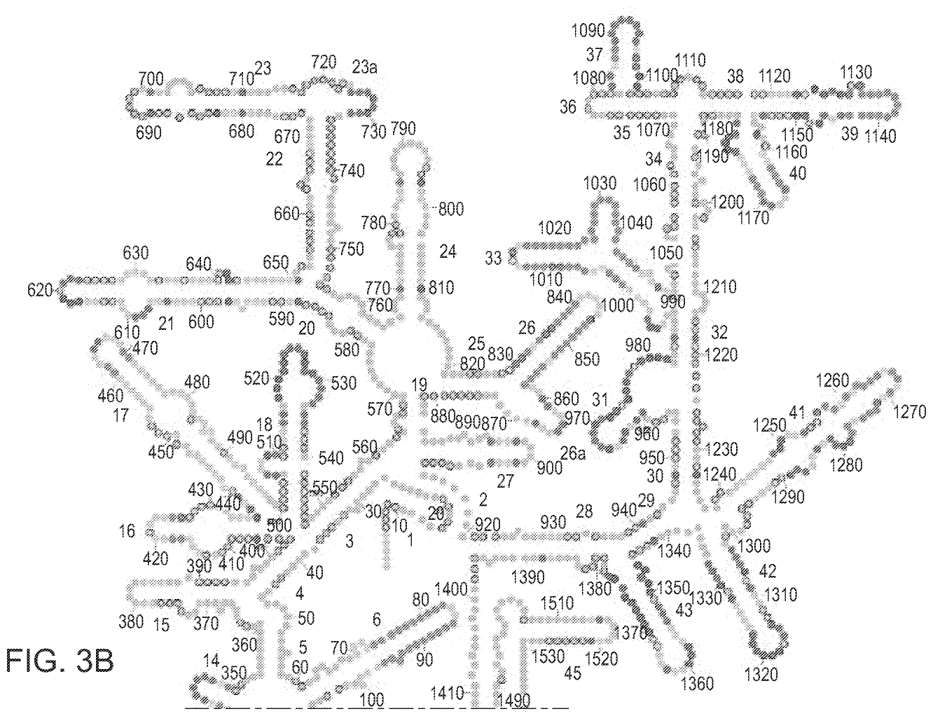

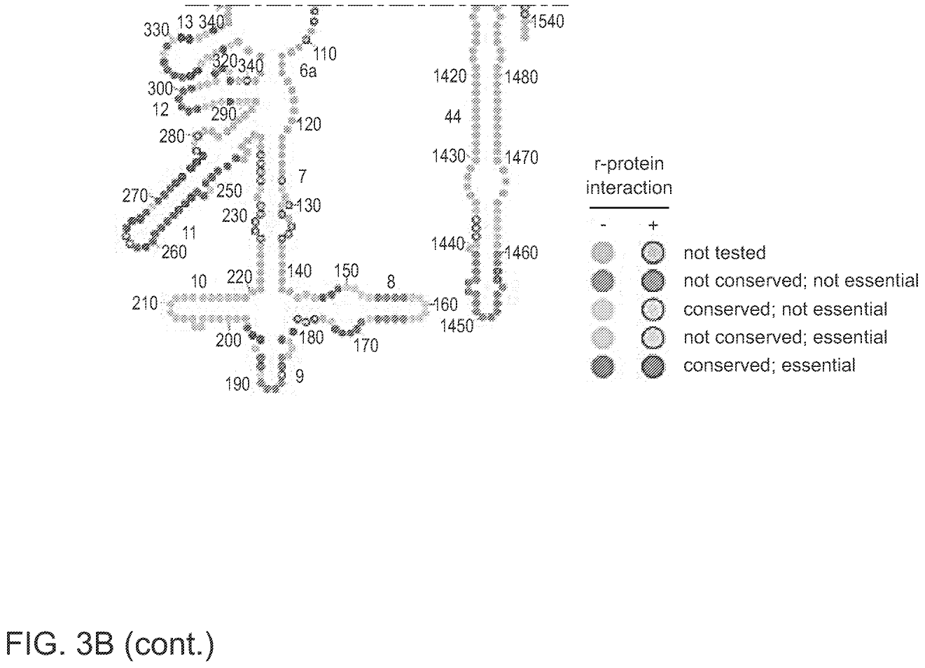

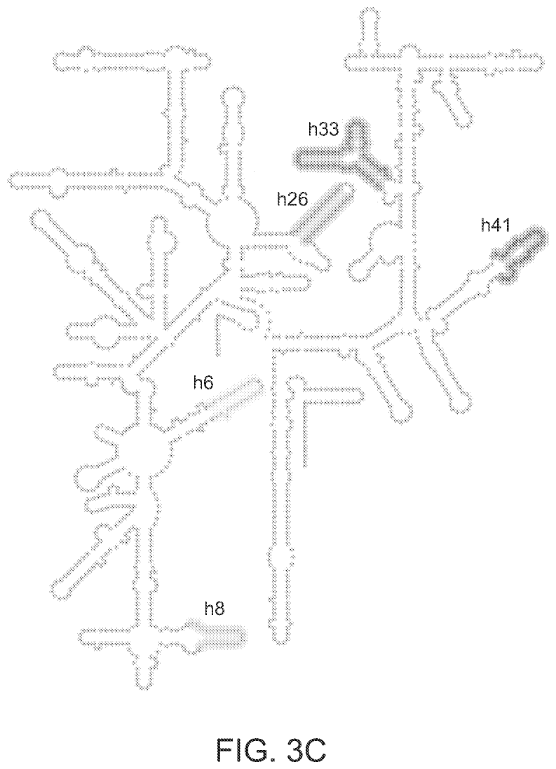

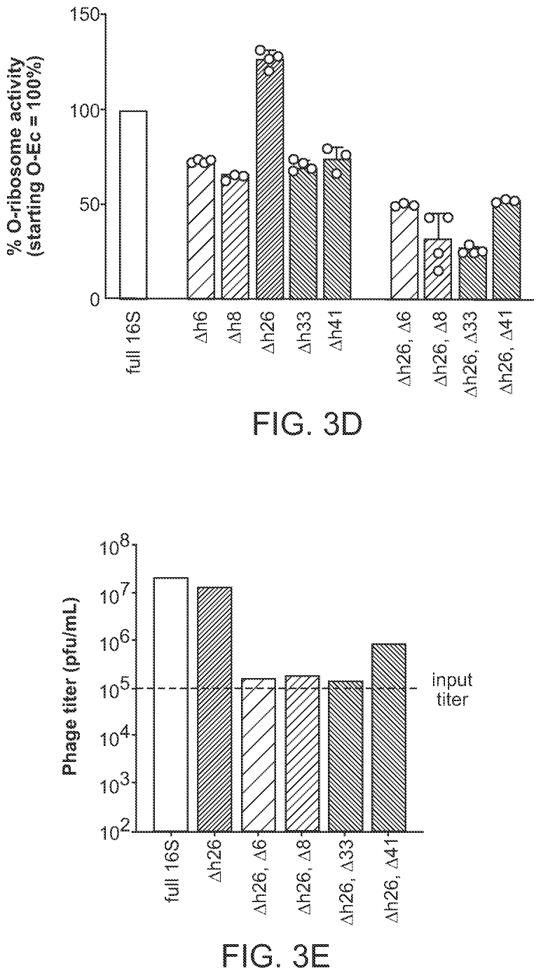

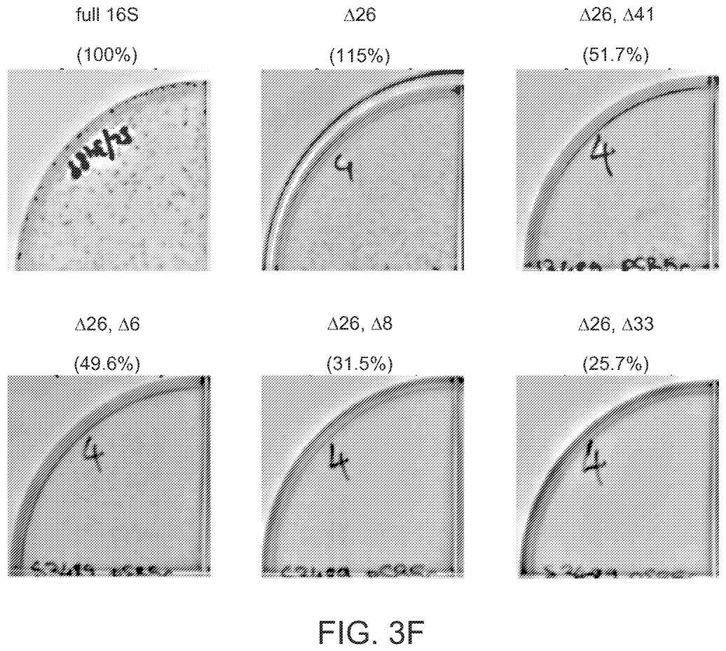

It has been recently shown that rRNAs derived from heterologous microbes can robustly support E. coli viability upon deletion of all host-derived rRNAs (Asai et al., 1999a: Kolber et al., 2021). As only E. coli-derived o-rRNAs have been successfully evolved to date, it was hypothesized that diverse heterologous o-rRNA sequences may undergo distinct evolutionary trajectories in PACE, yielding variable solutions to identical selection conditions. However, divergent heterologous ribosomes often suffer from reduced starting activity in an E. coli chassis as compared to wild-type E. coli ribosomes (Kolber, 2020). The 16S rRNA is highly conserved sequence, yet encoding poorly conserved residues often residing at the 3-dimensional periphery of the ribosome (FIG. 3A). To define a threshold for heterologous ribosome activity, deletions were generated in E. coli-derived 16S o-rRNA and their activity levels were characterized using reporter and SP enrichment assays (FIGS. 3B-3F). These experiments established that SPs bearing o-rRNAs with activity levels ≥32% of WT E. coli o-rRNA robustly propagated under stringent conditions.

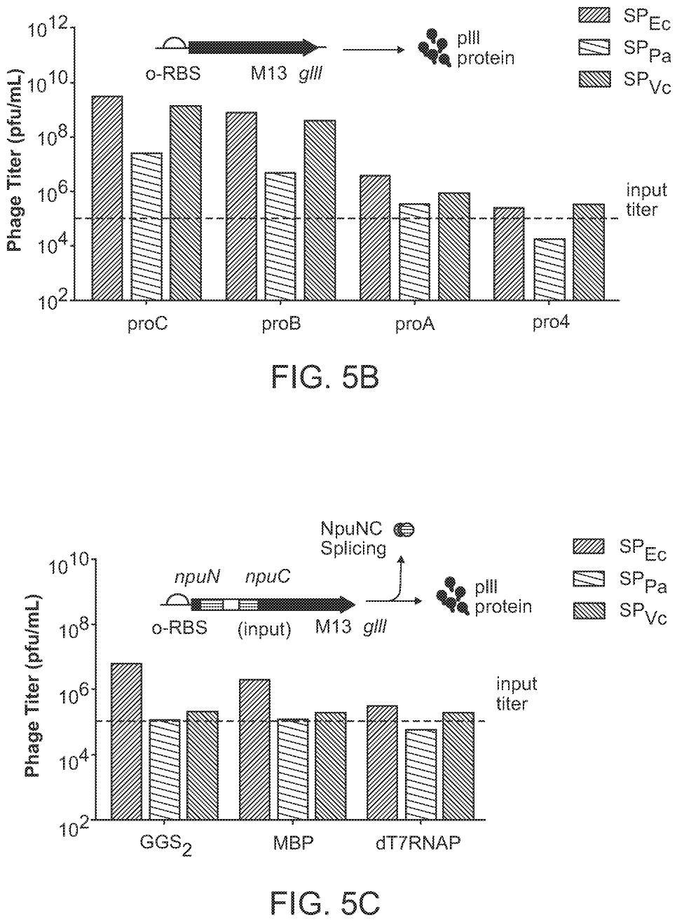

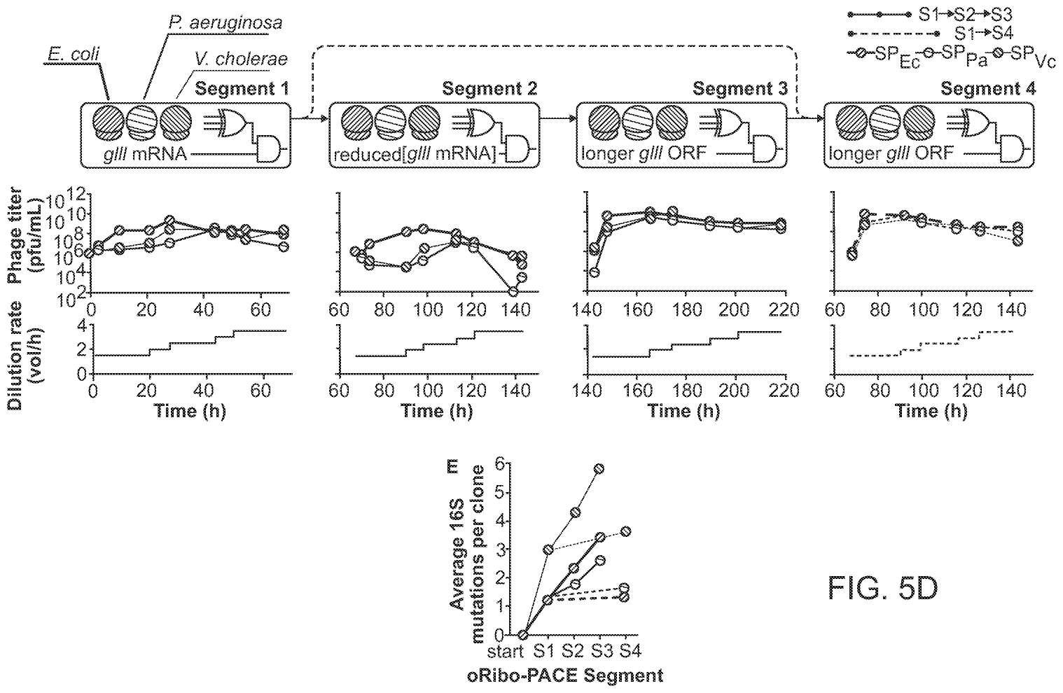

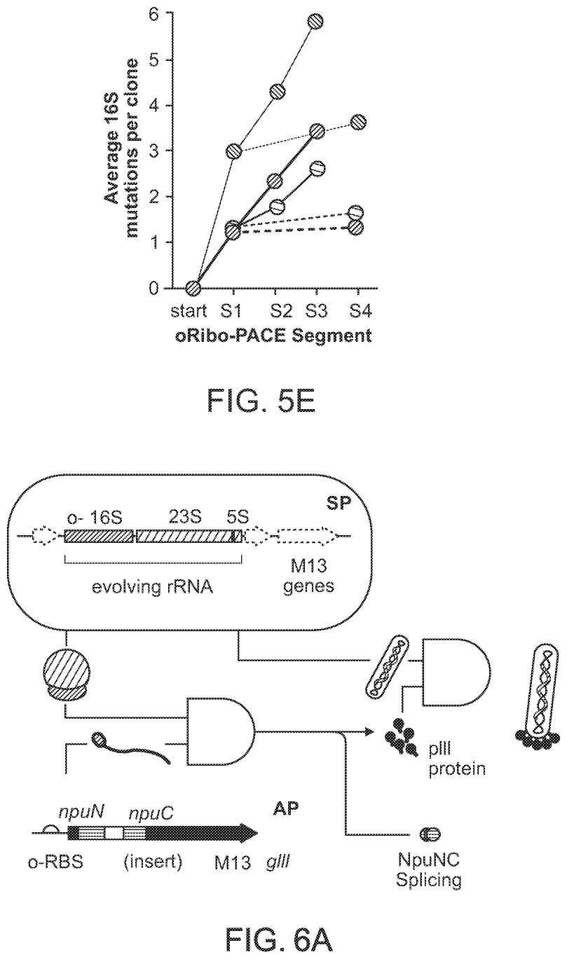

Next, P. aeruginosa (Pa) and V. cholerae (Vc) heterologous o-rRNAs were identified as promising candidates for oRibo-PACE, as they showed comparable activity to E. coli-derived o-rRNA (FIG. 5A) and could successfully propagate in standing culture, albeit at lower efficiency than their E. coli counterpart (FIG. 5B). To evolve heterologous rRNAs, starting rRNA species were subjected to multi-stage selection regimes with increasing selective pressure. 218 h (˜268 generations (Esvelt, Carlson and Liu, 2011)) of PACE was performed using E. coli (SP_{Ec}), P. aeruginosa (SP_{Pa}), and V. cholerae (SP_{Vc}) o-rRNAs while varying selection stringency over multiple segments (FIGS. 5B-D). In all segments, a previously optimized MP, MP6 (Badran and Liu, 2015a), was employed to enhance o-rRNA sequence diversity, and regularly increased lagoon flowrates were used to enhance selection stringency.

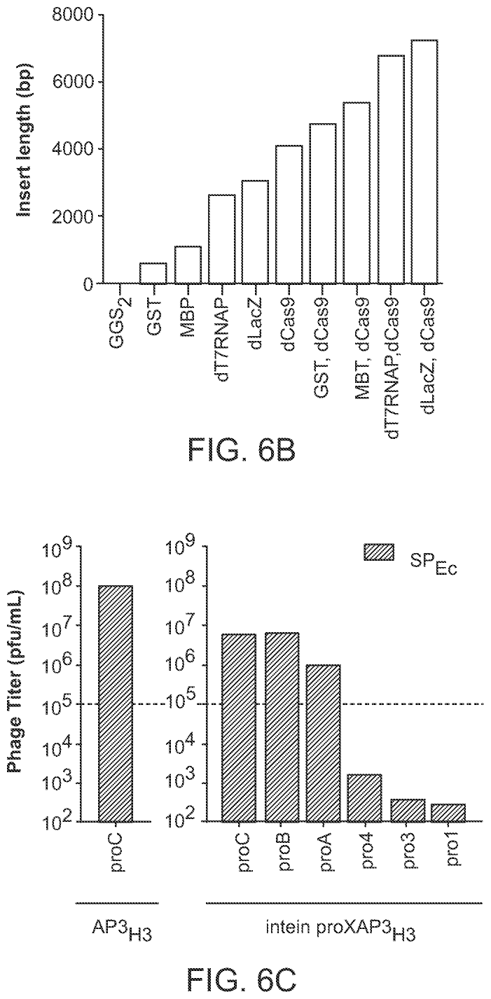

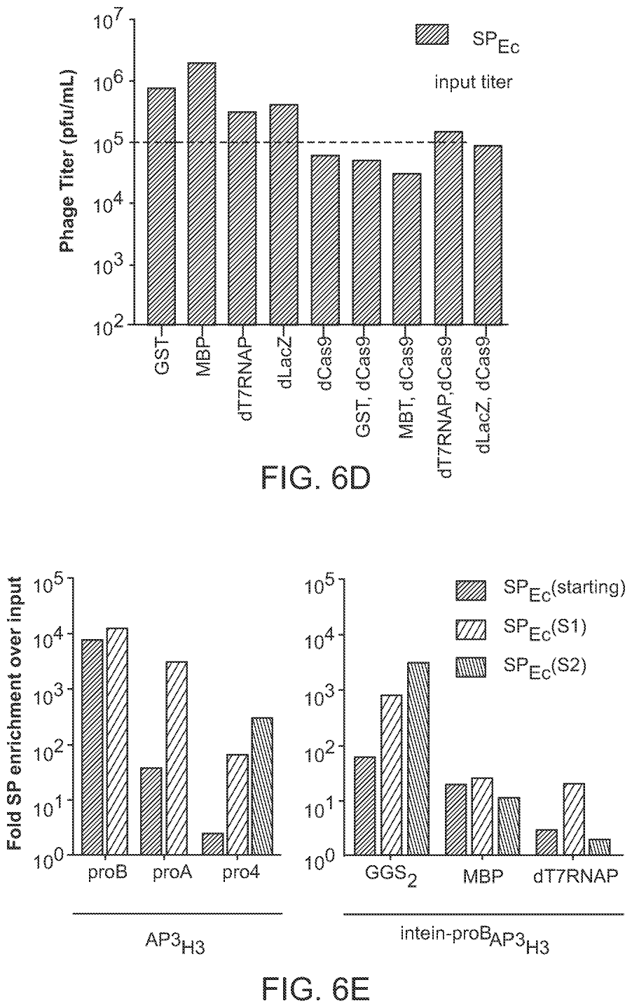

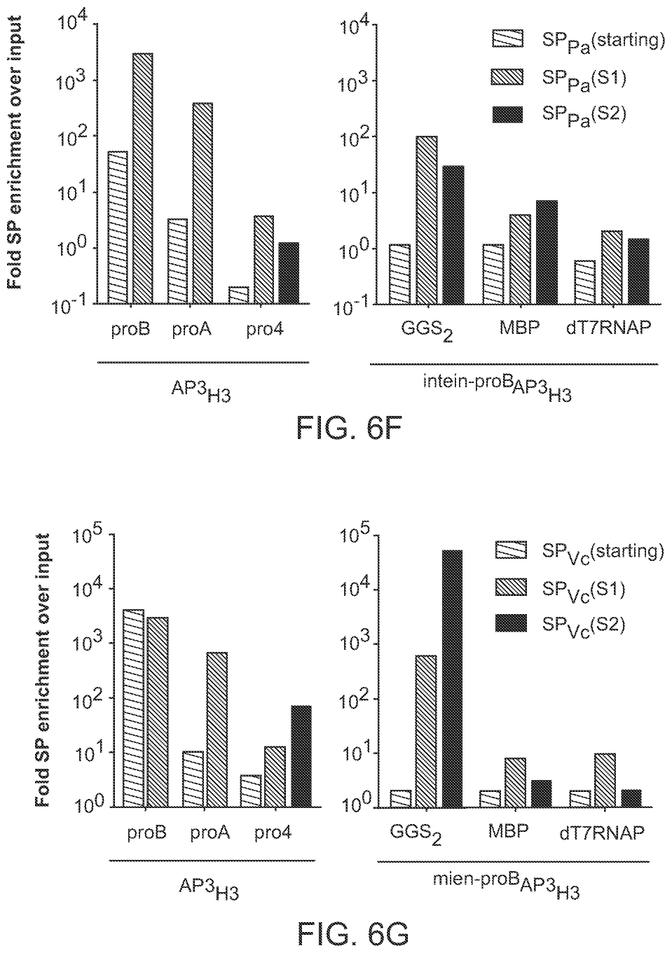

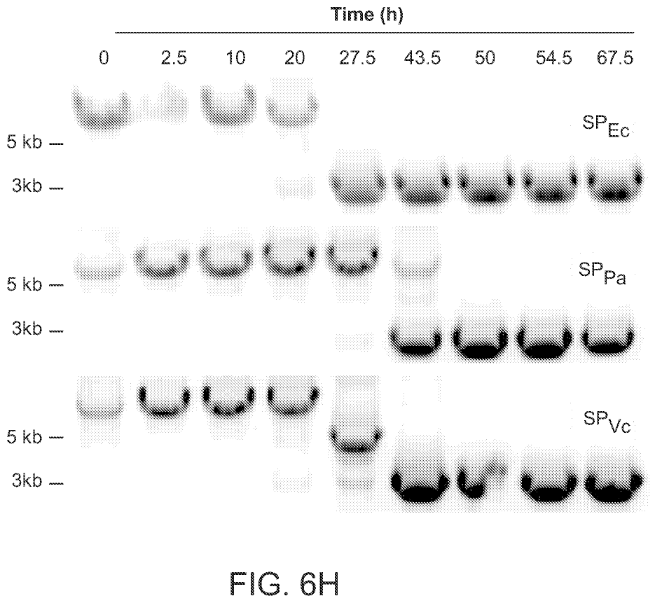

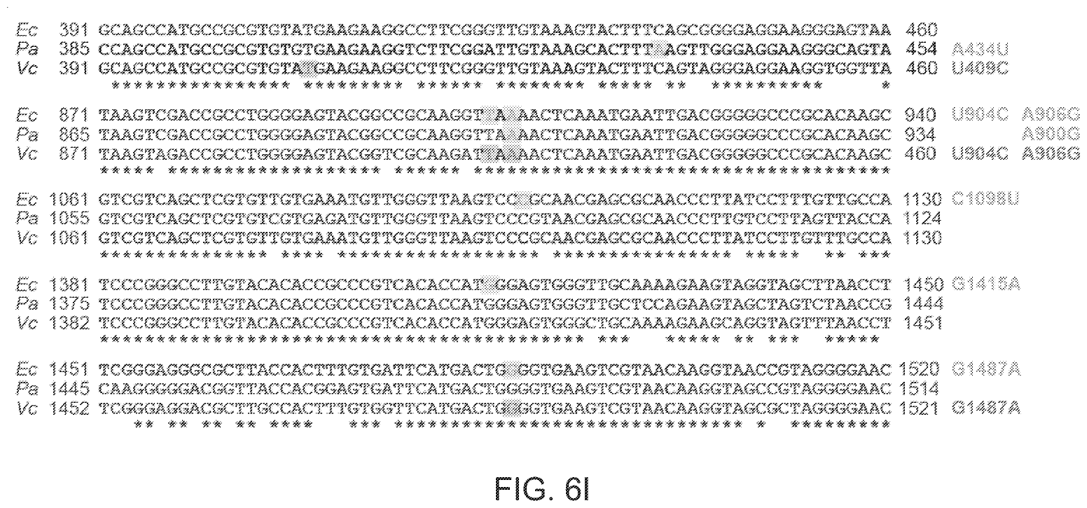

In the first segment (S1=0-68 h), the clonal SP-borne o-rRNAs were diversified through genetic drift by employing a constitutive promoter driving gIII expression from AP3_{H3} (proB (Davis, Rubin and Sauer, 2010): FIG. 5B, 5D). During the second segment (S2=68-143 h), election stringency was increased by reducing the gIII promoter strength 8-fold (Davis, Rubin and Sauer, 2010)): FIGS. 5B, 5D), which resulted in a >250-fold decrease in SP propagation efficiency (FIG. 5B). During the third segment (S3=143-218 h), a split-intein pIII (Wang et al., 2018) strategy was incorporated where an inserted protein sequence increased the effective length of gIII by 123% (425 to 947 amino acids) and decreased SP propagation efficiency further by >120-fold (FIG. 5C, 5D, FIGS. 6A-6G). Finally, to examine the effect of selection schedule on o-ribosome variant activities, a fourth segment (S4=68-143 h) was carried out using the split-intein pIII approach and SP populations immediately following genetic drift (S1→S4) to compare a shorter selection regime to the aforementioned longer version (S1→S2→S3) (FIG. 5D). It was noted that all SP populations robustly propagated across all segments, with the exception of SP_{Pa} during S2 (FIG. 5D, FIG. 6F), which rebounded during subsequent high stringency selection, suggesting accumulation of novel mutations to enable enhanced o-rRNA activities. Furthermore, all three SP populations underwent cognate 23S rRNA deletion at virtually identical time points during oRibo-PACE (FIG. 6H), reflecting complementation with the host E. coli 23S rRNA as previously described (Kolber et al., 2021).

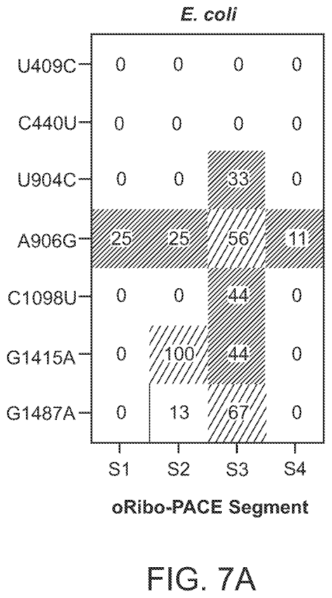

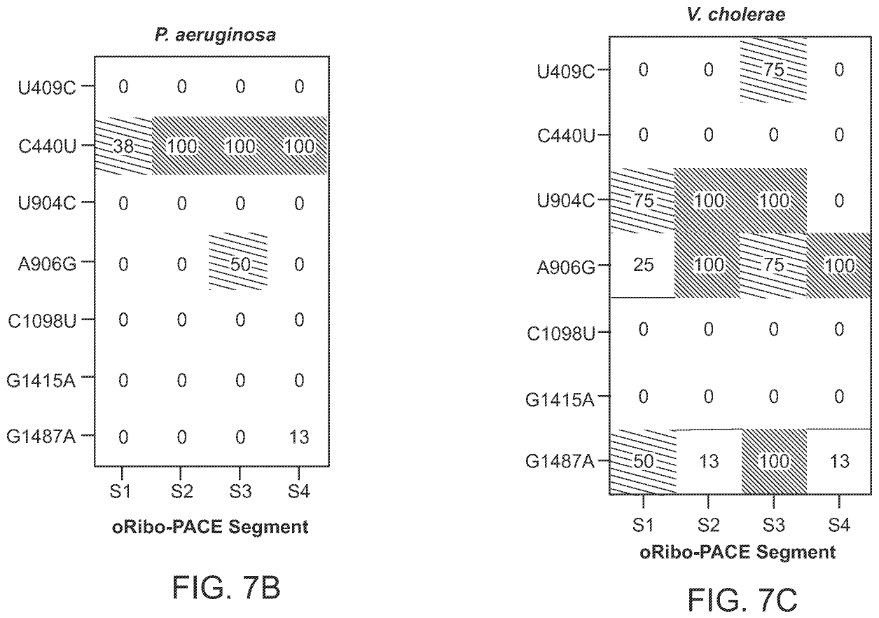

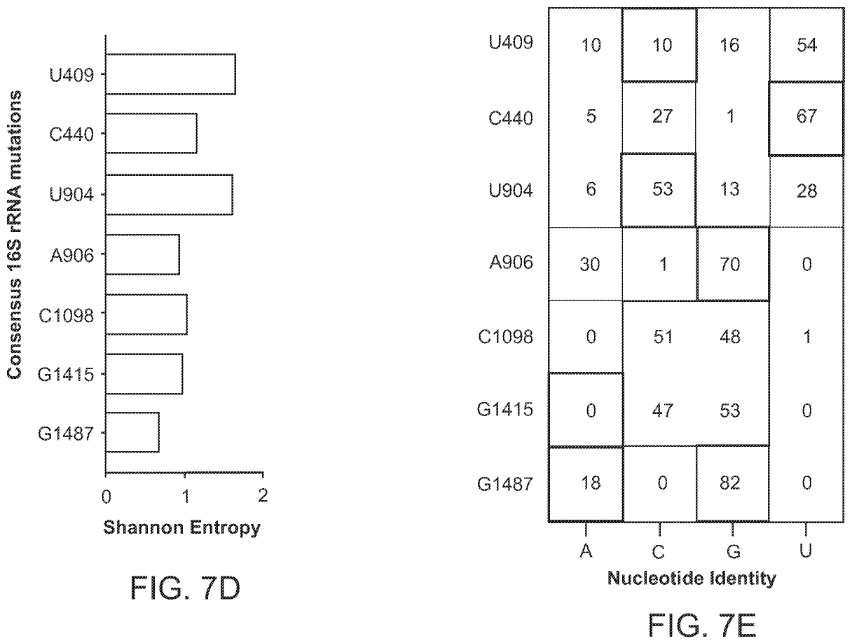

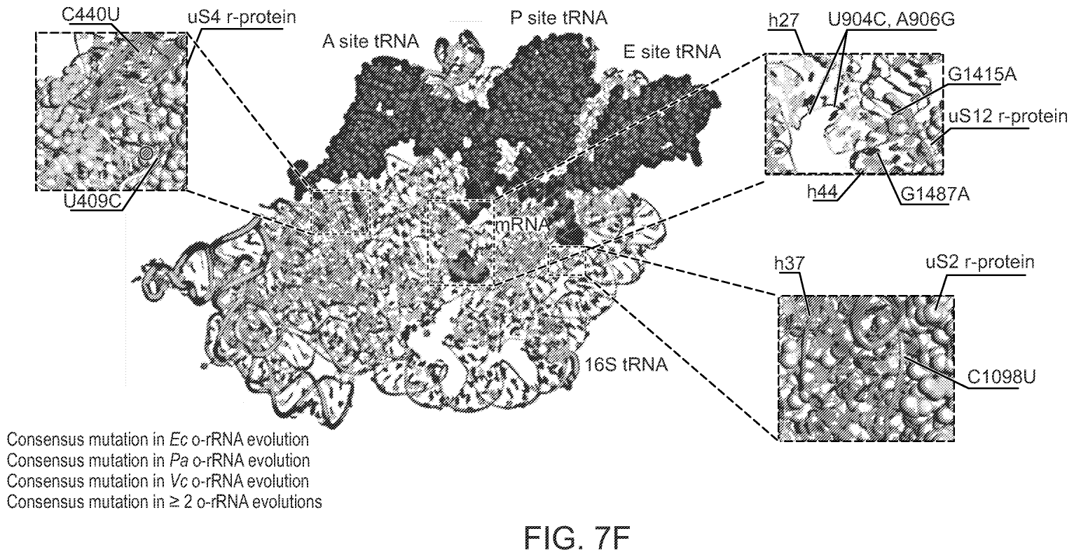

Individual clone sequencing at the end each segment revealed sweeping mutations in all SP-borne o-rRNAs (FIG. 6I, FIGS. 13-15). Collectively, V. cholerae o-rRNAs developed the highest average number of mutations per clone throughout all segments, while P. aeruginosa o-rRNA retained the lowest number of mutations (FIG. 5E). This trend is consistent with propagation efficiencies of the corresponding SPs during oRibo-PACE (FIG. 5D). A number of unique mutations became prevalent in each SP population at varying segments: C1098U (E. coli, S3), G1415A (E. coli, S1), U409C (V. cholerae, S3) and A434U (P. aeruginosa, S1) (FIGS. 7A-7C, FIG. 6I, FIGS. 13-15). Interestingly, varying levels of natural sequence conservation were noted at the discovered sites (FIGS. 7D, 7E) (O'Connor and Dahlberg, 2001), which indicated that mutations at these positions may not necessarily indicate functional relevance.

Notably, an identical mutation in h27 was evolved independently in all o-ribosomes at different segments: A906G in E. coli and in V. cholerae (S1), and A900G in P. aeruginosa (S3) (FIGS. 7A-C, 7F, FIG. 6I, FIGS. 13-15). Two identical mutations were also found in the E. coli (U904C, G1487A) and V. cholerae (U904C, G1488A) populations (FIGS. 7A-C, 7F, FIG. 6I, FIGS. 13-15). A906 and U904 (helix 27, E. coli numbering) together with G1487 (h44) form an interface with protein uS12 (FIG. 7F) during tRNA selection and ensure translation accuracy (Vila-Sanjurjo et al., 2003: Alksne et al., 1993: Agarwal, Gregory and O'Connor, 2011). The current observation of three converged mutations (U904C, A906G, and G1487A) in the E. coli and V. cholerae populations indicated adaptive evolution towards enhanced translational output (FIG. 3D). The E. coli-only mutation C1098U (h37) interacts with r-protein uS2 (Brodersen et al., 2002) (FIG. 7F) during final S30 subunit assembly (Moll et al., 2002), whereas G1415A is proximal to G1487A (h44, FIG. 7F) and may influence tRNA selection. The V. cholerae-only mutation U409C forms a wobble base pair with G433 (h16) interacts with us4, where its mutation to a cytosine may yield a stronger C409-G433 Watson-Crick pair (FIG. 7F) (Brodersen et al., 2002). The P. aeruginosa-only mutation A434U (h17) is near the binding site of protein uS4 (Agarwal et al., 2015) (FIG. 7F). Taken together, these results showcase hallmarks of both similar and independent evolutionary trajectories to overcome identical selection regimes.

### Example 4: PACE-Derived o-rRNAs Exhibited Augmented Translation Rates

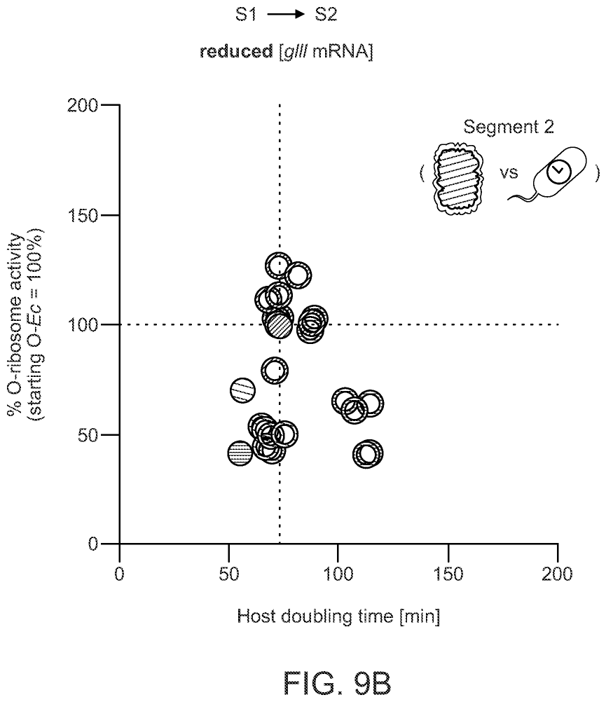

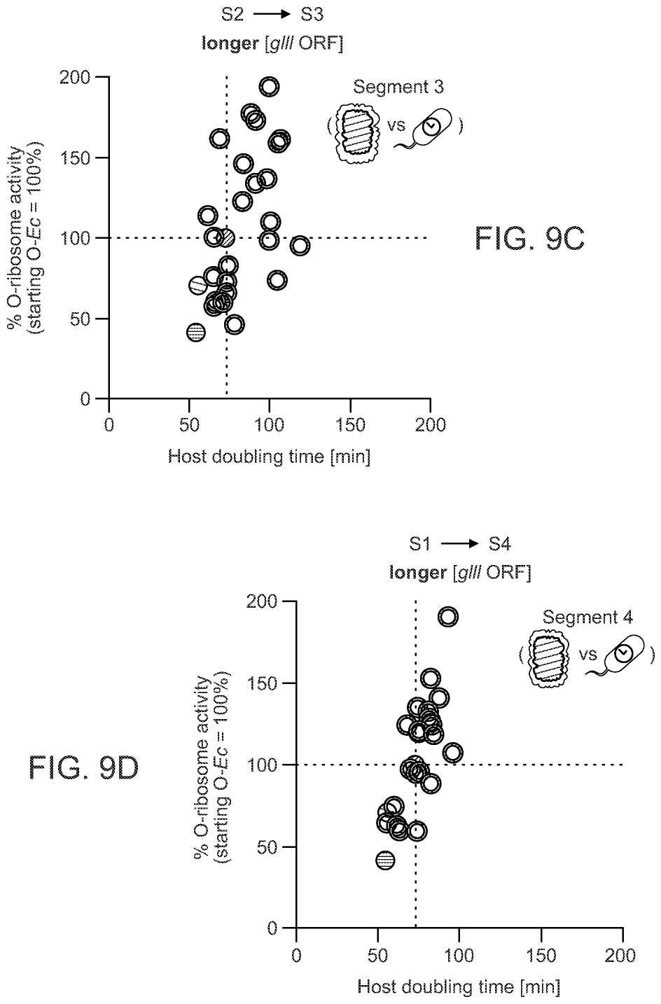

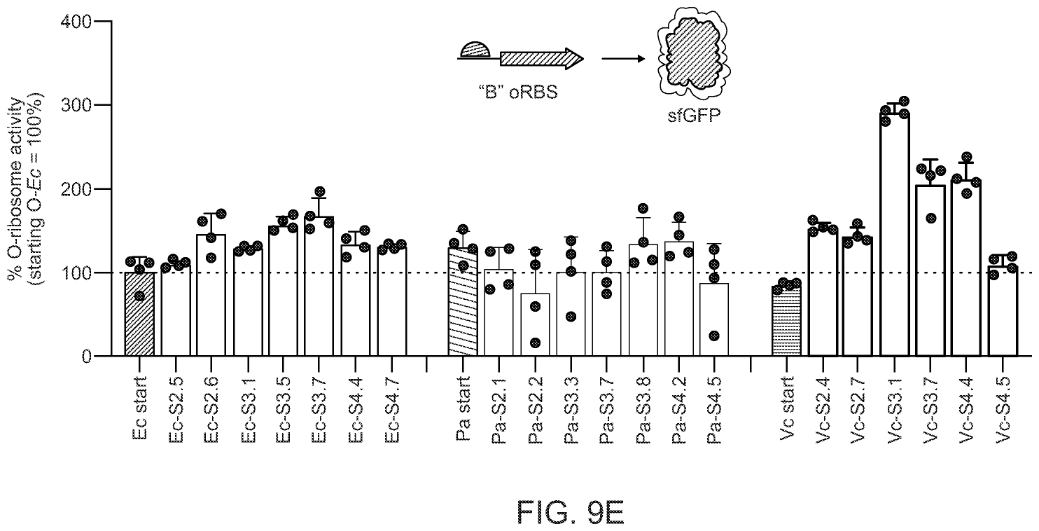

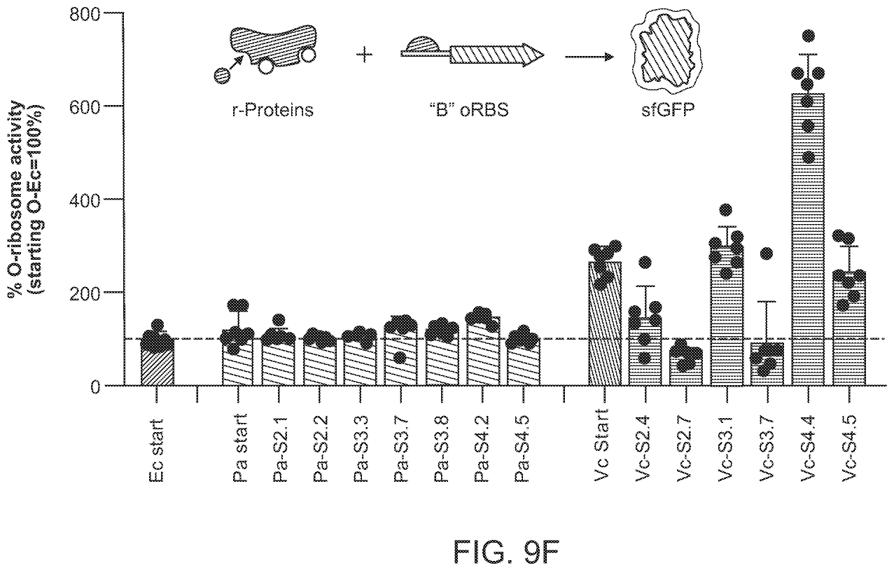

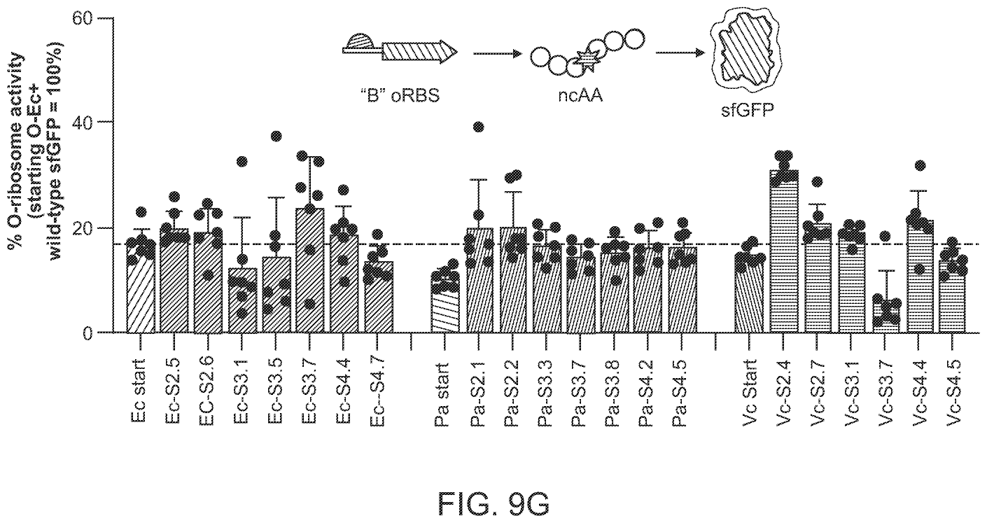

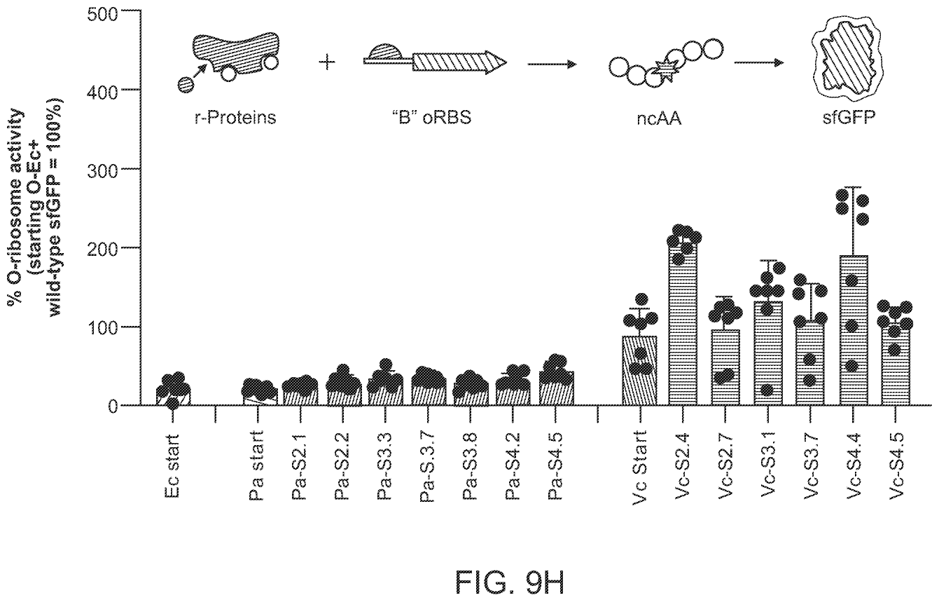

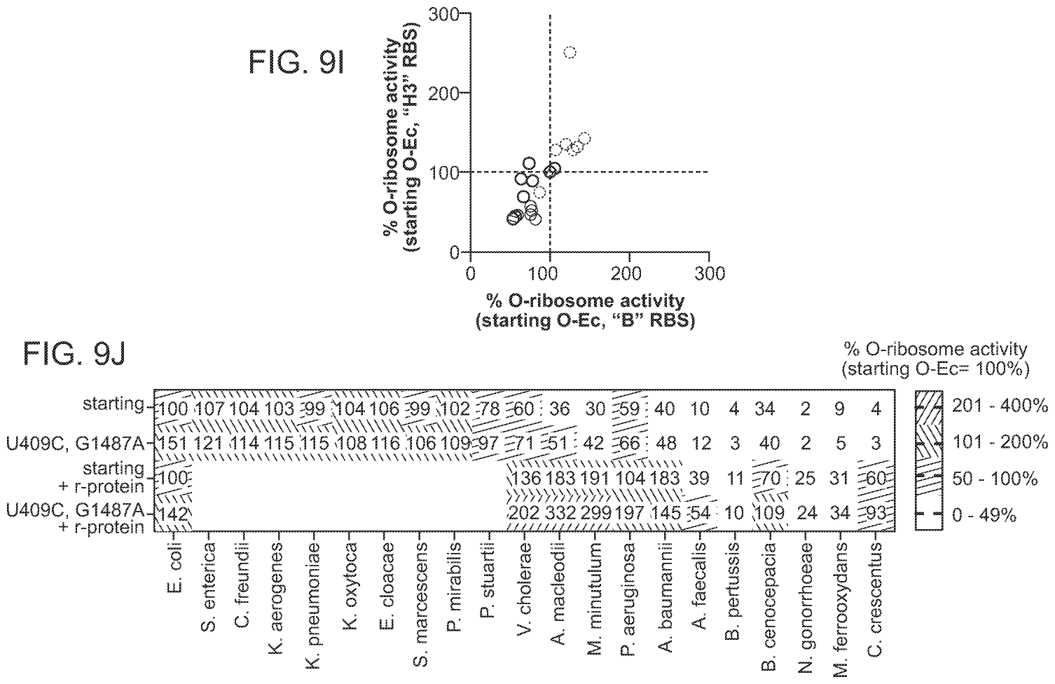

To assess the consequences of PACE-derived mutations on o-ribosome function, evolved o-rRNAs were subcloned into inducible expression plasmids (EPs) and their activities were evaluated in vivo using a battery of assays: (1) characterizing translation rate using orthogonal cellular reporter proteins (Kolber, 2020), (2) quantifying host E. coli growth burden (Darlington et al., 2018) during o-ribosome overproduction, (3) investigating possible context dependence effects on translation by using the unrelated “B” o-RBS/o-antiRBS system (Rackham and Chin, 2005), (4) analyzing preferential use of E. coli host factors by evolved heterologous o-ribosomes via complementation with cognate ribosomal proteins (Kolber, 2020), (5) exploring improvements in genetic code expansion through non-canonical amino acid (ncAA) incorporation (Bryson et al., 2017), and (6) analyzing context independence of evolved consensus mutations in unrelated, divergent heterologous rRNAs comparing everything to starting E. coli o-rRNA under the same conditions (FIG. 9A).

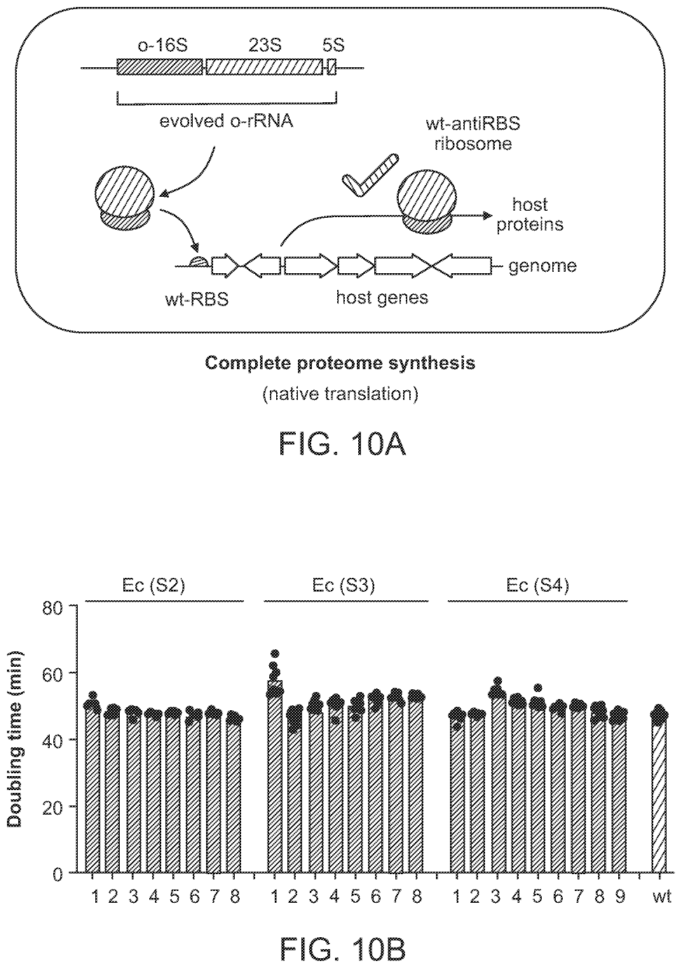

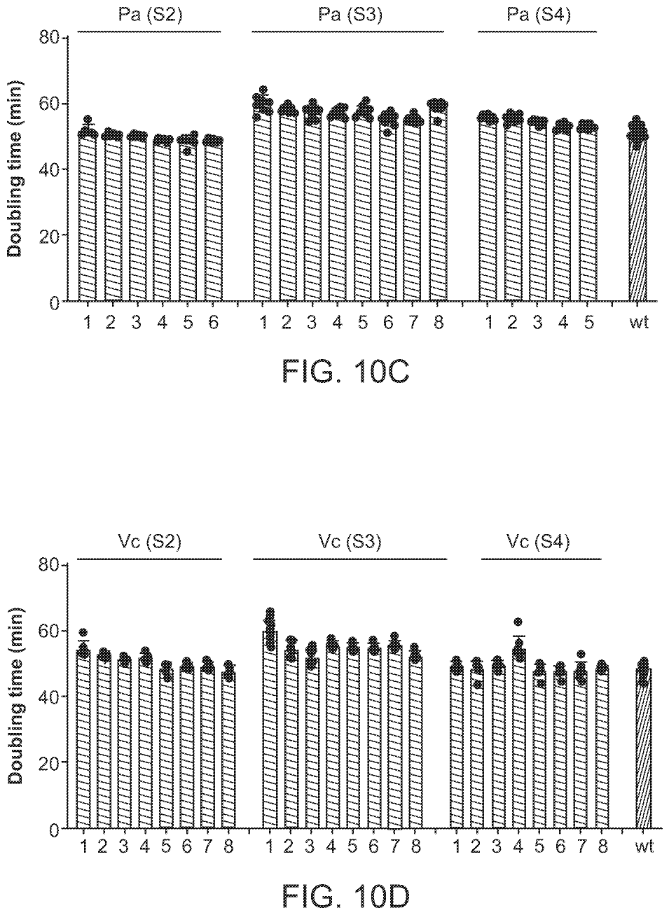

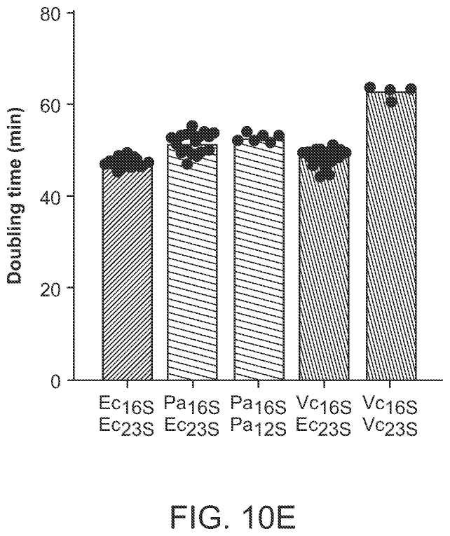

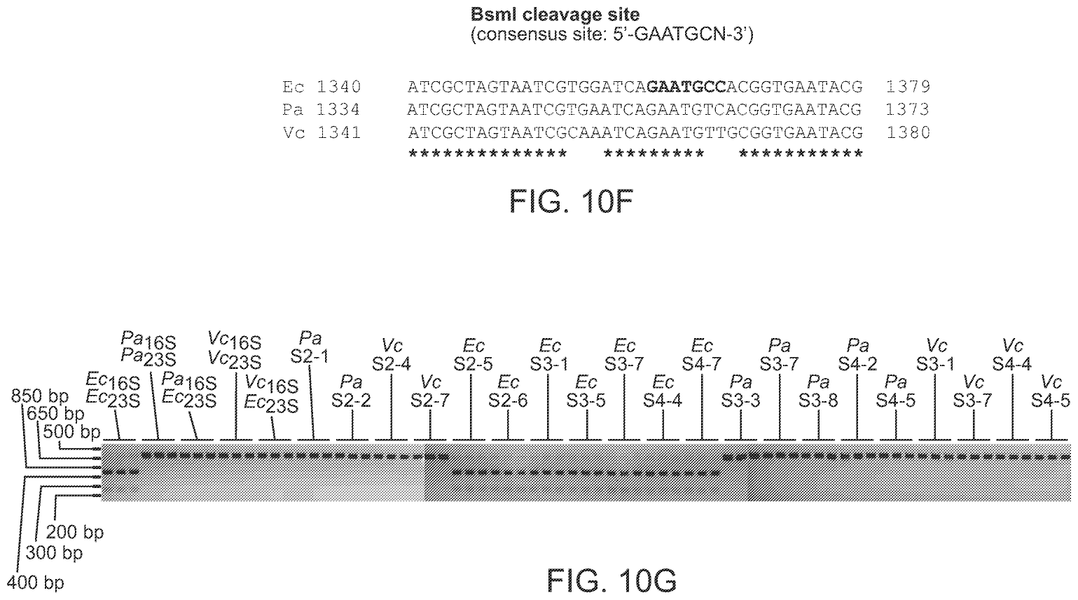

Continuous monitoring of luminescence activity was used as a real-time proxy of translation rate for o-ribosomes. Using kinetic luminescence output at fixed optical densities (OD₆₀₀=0.15, FIG. 10A), minimal activity improvements of evolved E. coli o-rRNA variants from S2 and S4 were observed. However, the six variants isolated from the longer S3 trajectory showed higher (146-196%) activity compared to the starting E. coli o-rRNA (FIGS. 9B-9D, FIG. 10B). Only a single P. aeruginosa o-rRNA variant evolved after 218 h (S3) of PACE yielded similar activity to starting E. coli (96%) luminescence output (FIG. 9C, FIG. 10C). Remarkably, almost half ( 11/24) of V. cholerae 16S o-rRNA variants produced higher (109-186%) activities relative to E. coli (FIGS. 9B-D, FIG. 10D). These observed differences in evolved o-rRNA populations, which do not correlate with 16S sequence identity to E. coli (P. aeruginosa: 85%: V. cholerae: 90%), suggest that heterologous rRNA choice may affect directed evolution campaign success by as yet unclear determinants.

Orthogonal ribosomes are known to negatively affect host cell fitness, likely due to over-commitment of resources to the production of supplementary ribosomes 276 (FIG. 10E) (Kolber et al., 2021). A previously reported burden for E. coli o-rRNA expression on host cells (Darlington et al., 2018) was observed, and in some cases these effects were moderately amplified in evolved variants (FIG. 9B-D, FIG. 10E-H). In general, host doubling time increased for o-rRNA mutants with respect to starting o-rRNAs ( 61/67 mutants: 91%) and this trend held for o-rRNA mutants with enhanced o-ribosome activity as compared to starting scaffold ( 49/53 mutants: 92.5%) (FIGS. 9B-D). However, expressing wild-type or evolved P. aeruginosa or V. cholerae o-rRNAs exerted a lighter metabolic burden on the E. coli host than expressing the corresponding E. coli o-rRNAs in many cases (FIGS. 9B-9D, FIGS. 10F-10H).

Representative variants from each rRNA origin and evolution segments were selected for further evaluation based on kinetic luminescence output. Using an orthogonal superfolder GFP (sfGFP) reporter, the highest o-ribosome activity (sfGFP yield) was observed from V. cholerae rRNA mutants (FIG. 9E). Further, it was hypothesized that E. coli r-proteins may show limited ability to catalyze heterologous ribosome assembly with rRNAs sufficiently divergent to that of E. coli, limiting overall functionality of the P. aeruginosa- and V. cholerae-derived o-rRNAs. To explore this, o-rRNA variants were complemented with cognate r-proteins which have previously been shown can improve heterologous activity (Kolber et al., 2021). r-Protein complementation of P. aeruginosa (using bS16, bS20) and V. cholerae (using bS1, uS15, bS16, bS20) o-rRNAs showed greatly increased sfGFP production as compared to the starting E. coli o-rRNA, corresponding to 122-147% and 146-629%, respectively (FIG. 9F). These findings show that oRibo-PACE-derived o-rRNAs evolved to overcome the designed selection pressure and did not appreciably adapt to the E. coli host.

Orthogonal translation systems have been employed to improve genetic code expansion efforts (Neumann et al., 2010), yet no reports have extended these capabilities to heterologous ribosomes. Therefore, select evolved o-rRNAs were evaluated for ncAA incorporation by integrating an amber (UAG) stop codon in sfGFP (residue Y151 (Chatterjee et al., 2014)) and assessed N_{ε}-((tertbutoxy) carbonyl)-L-lysine (Bock) incorporation using an established Methansarcina barkeri-derived tRNA-synthetase pair (Bryson et al., 2017). E. coli-derived o-rRNA mutants showed a no significant increase in Bock incorporation over starting E. coli o-rRNA (FIG. 9G). In the absence of cognate phylogenetically divergent r-proteins, P. aeruginosa and V. cholerae o-rRNA also resulted in negligible improvements in ncAA incorporation over starting E. coli (FIG. 9G). However, upon supplementation with cognate r-proteins, P. aeruginosa and V. cholerae-derived evolved o-rRNAs improved ncAA incorporation efficiency up to 45% and 209%, respectively (FIG. 9H). Context dependence of translation initiation was also evaluated by expressing sfGFP containing either B or H3 o-RBS, where a nearly uniform correlation and clustering by species was observed (FIG. 9I). It was noted that only E. coli-derived o-rRNA variants showed significant improvements in both B and H3 o-RBS contexts (FIG. 9I).

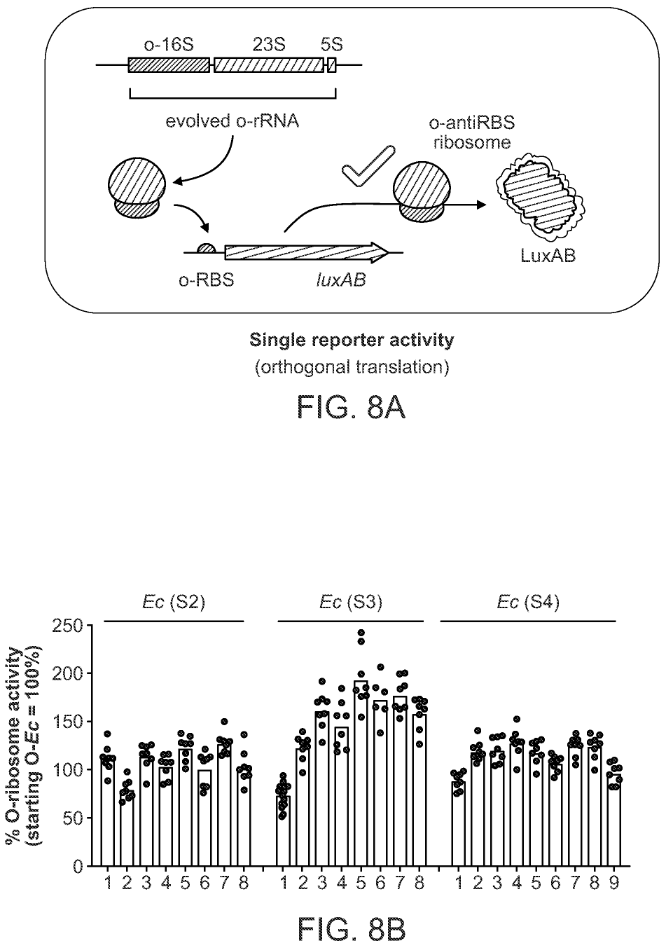

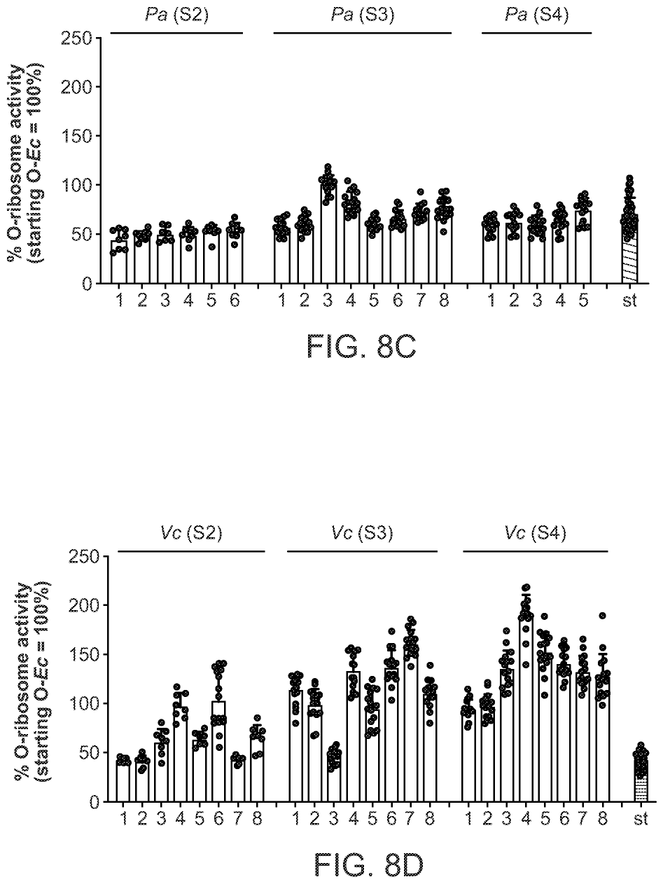

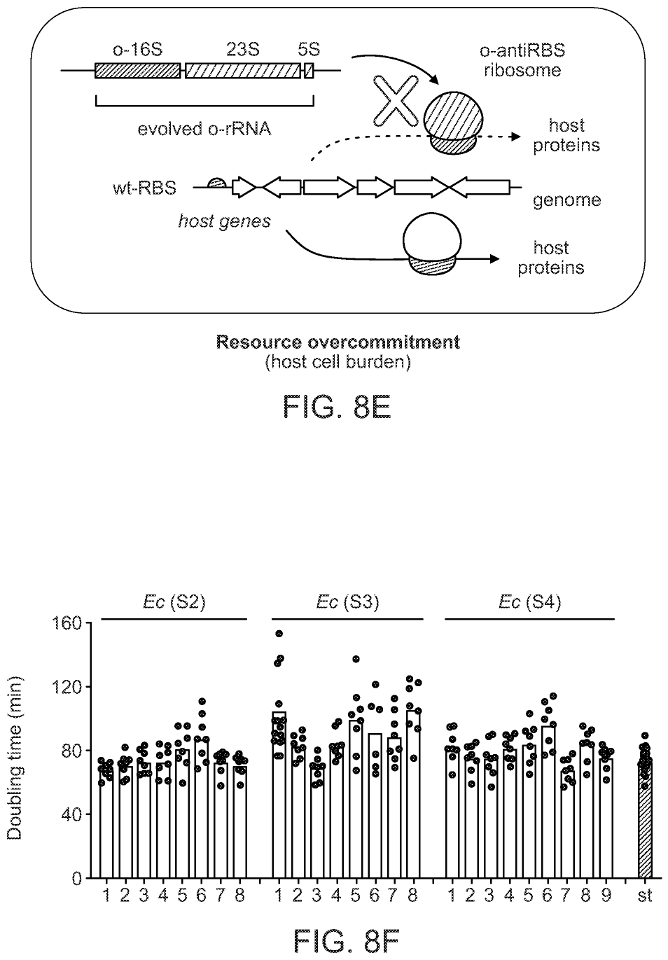

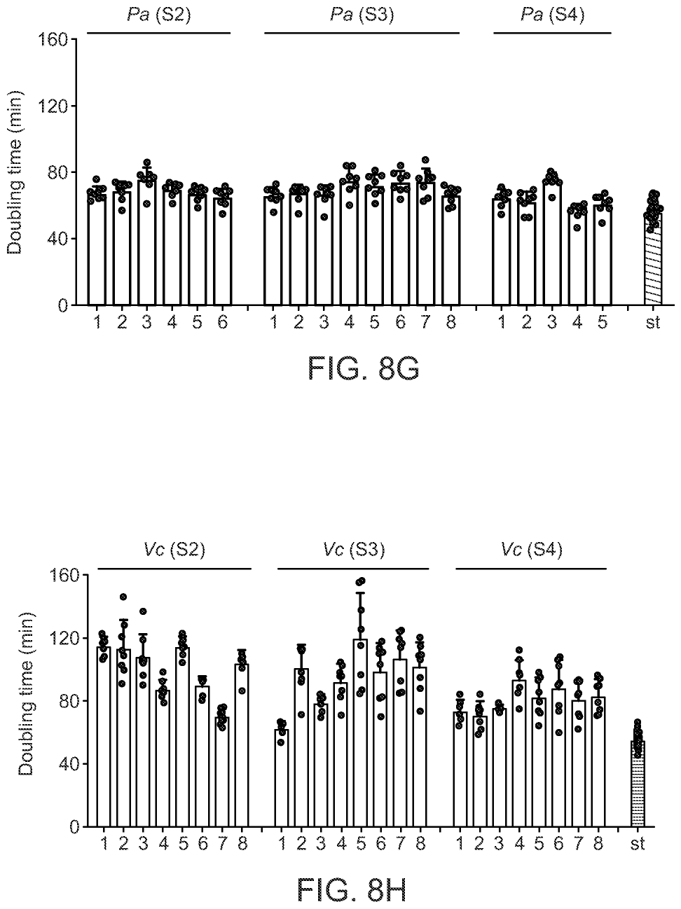

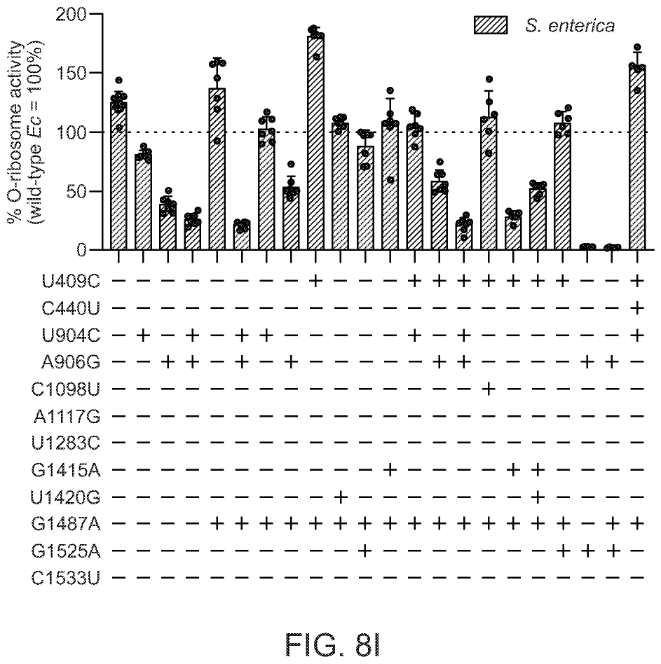

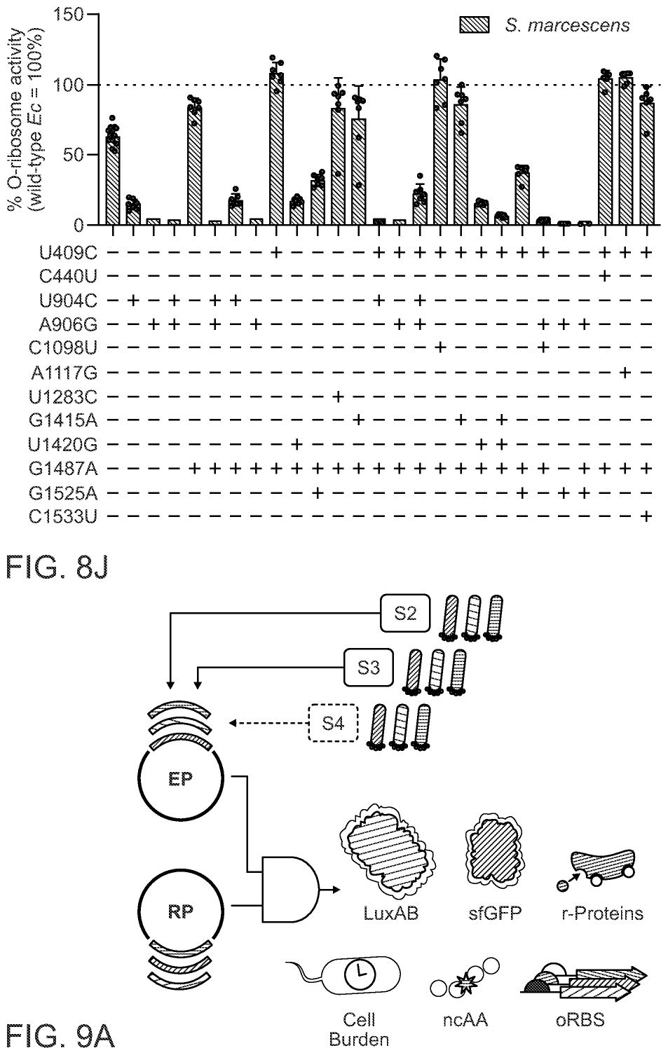

Finally, the functional relevance of mutations observed with high frequency during the various oRibo-PACE campaigns was explored. Through singular and combinatorial mutations using two unrelated heterologous o-rRNAs (Salmonella enterica and Serratia marcescens o-rRNAs), the two consensus mutations U409C and G1487A were uncovered as improving the kinetic capabilities of orthogonal ribosomes (FIG. 8I, 8J). Interestingly, this mutational combination was only observed in the V. cholerae campaign, which typically showed greater activities than the E. coli and P. aeruginosa counterparts across all assays. Both consensus mutations were transplanted into o-rRNAs from increasingly divergent microbes, which resulted in general improvements to translation activities (FIG. 9J). This effect was amplified when tested alongside the cognate r-proteins (FIG. 9J). Excitingly, o-rRNAs from Alteromonas macleodii and Marinospirillum minutulum increased activity up to 332% and 299 as compared to the starting E. coli o-rRNA scaffold, respectively (FIG. 9J). Cumulatively, these extensive analyses demonstrated that oRibo-PACE-derived o-rRNAs enabled the discovery of context independent mutations that broadly improved o-ribosome activities.

### Example 5: Kinetically Enhanced rRNAs Limited Population Growth Rate Via Reduced

### Fidelity

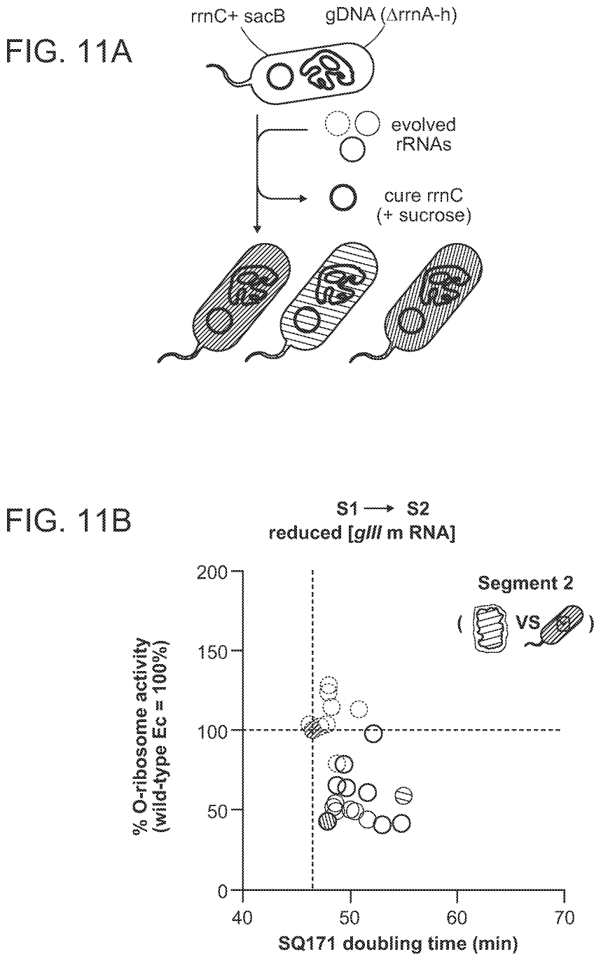

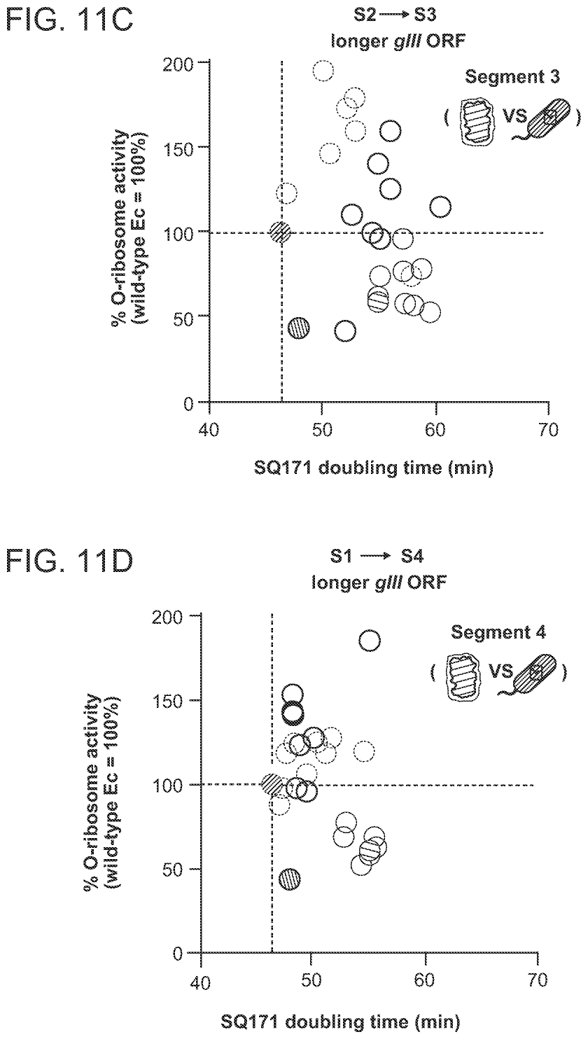

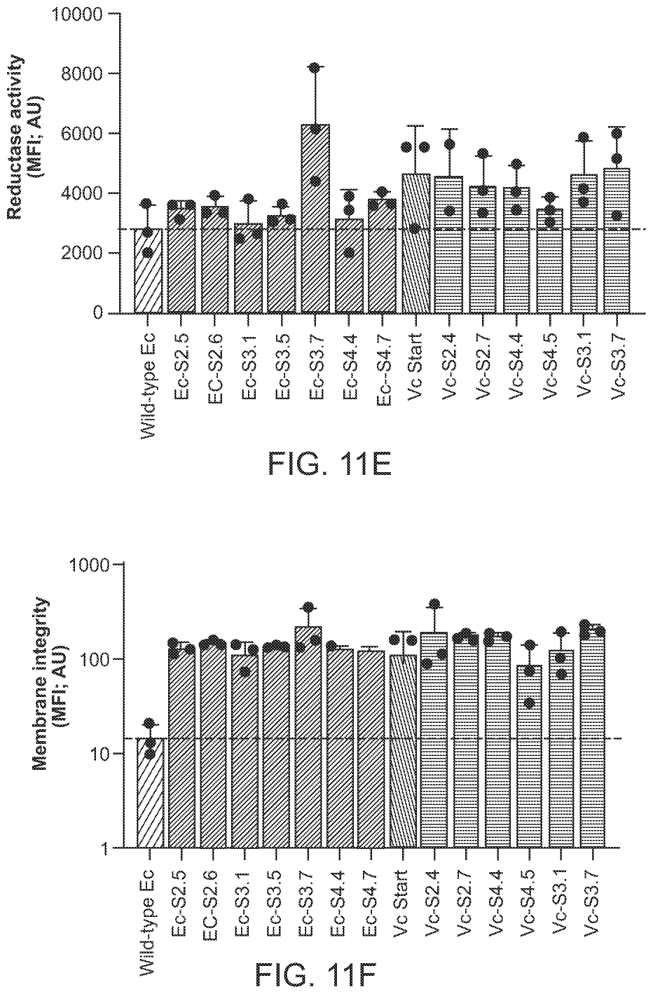

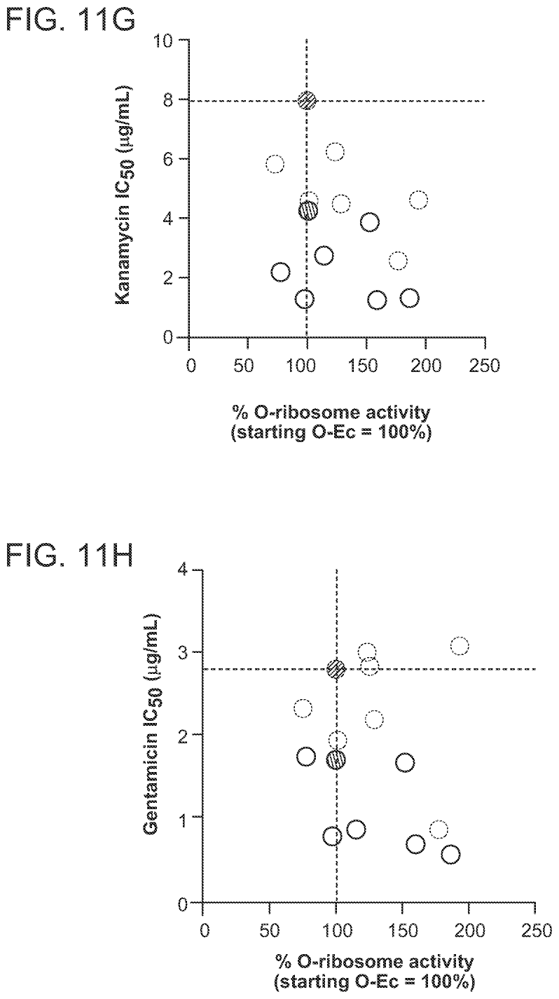

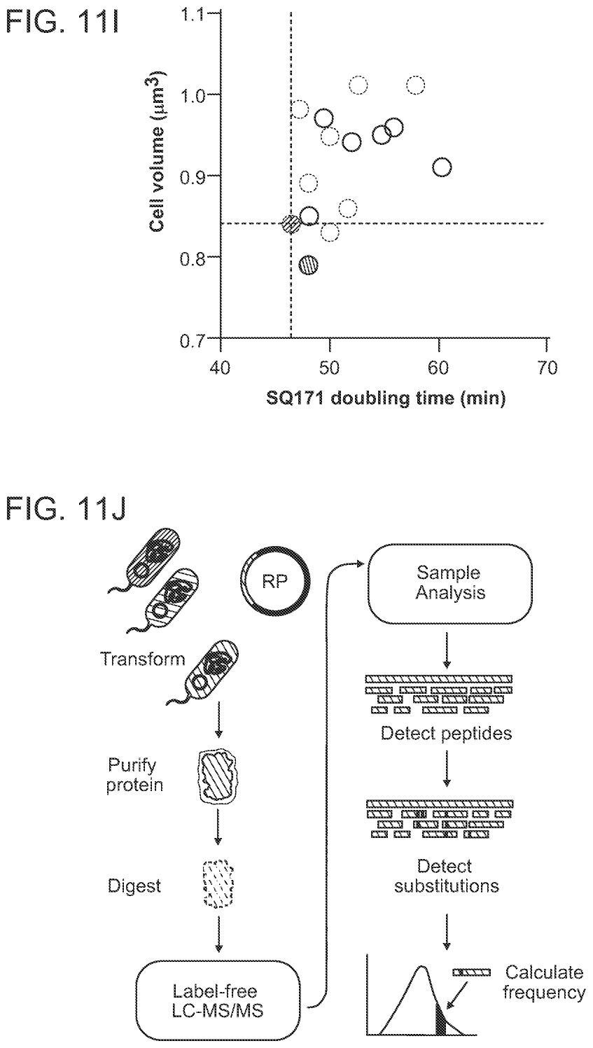

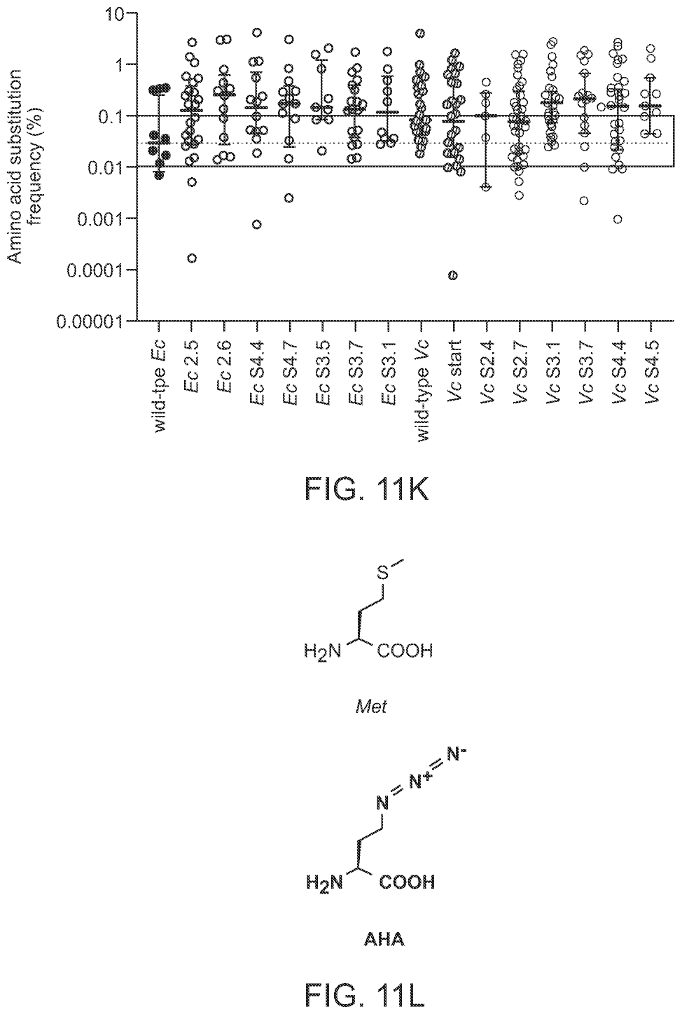

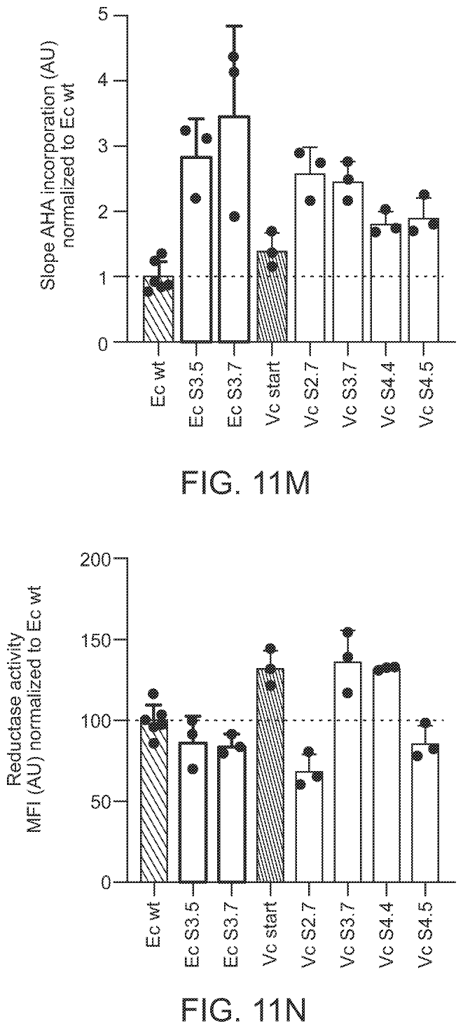

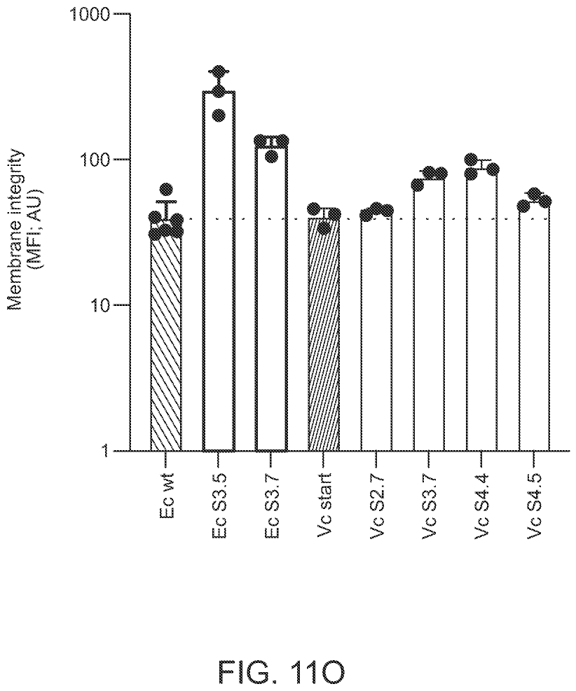

Analyses of evolved o-rRNA activities indicated that oRibo-PACE can robustly influence ribosome translational kinetics in engineered settings. To elucidate the physiological cost of kinetically-enhanced rRNA variants, the wild-type antiRBS sequence was introduced into evolved o-rRNAs and their ability to complement the rRNA efficiency of SQ171 E. coli cells and translate all cellular proteins was assayed (FIG. 11A, FIG. 10A) (Asai et al., 1999a).

In all cases, evolved 16S rRNAs robustly complemented the ribosomal deficiency of this strain (used alongside native E. coli 23S, 5S: FIGS. 11B-11D, FIGS. 10B-10F). It was noted that all evolved variants from oRibo-PACE S3 and S4 that exhibited improved luminescence output (>145% of respective wildtype) showed a concomitant proliferation rate reduction in SQ171 cells (FIGS. 11C, 11D): E. coli (6-12% reduction), P. aeruginosa (11% reduction), V. cholerae (7-24% reduction). These observations, which were unexpected given the predicted interplay between translational kinetics and bacterial cell proliferation, motivated further exploration of biophysical and cellular outcomes of evolved rRNAs.

E. coli ribosome content and therefore translation rate is thought to correlate with cell proliferation (Serbanescu, Ojkic and Banerjee, 2020), yet kinetically evolved rRNA variants did not result in faster proliferating strains. To address this, all E. coli and V. cholerae SQ171 strains were assessed for cell vitality in nutrient rich growth conditions (Davis Rich Medium, DRM) (Carlson et al., 2014a). Analysis of cellular respiration through measurement of electron transport chain function (reductase activity) is a reliable marker of vitality (Cologgi et al., 2011). By assessing the reductase activity and co-staining with propidium iodide, a membrane integrity marker, using all E. coli and V. cholerae mutants, comparable reductase activity between all strains (FIG. 11E) was observed with indications of minor compromises in membrane integrity in all strains as compared to wild-type E. coli (FIG. 11F). Further analyses using P. aeruginosa derived ribosomes were not pursued due to poor overall activities across most assays.

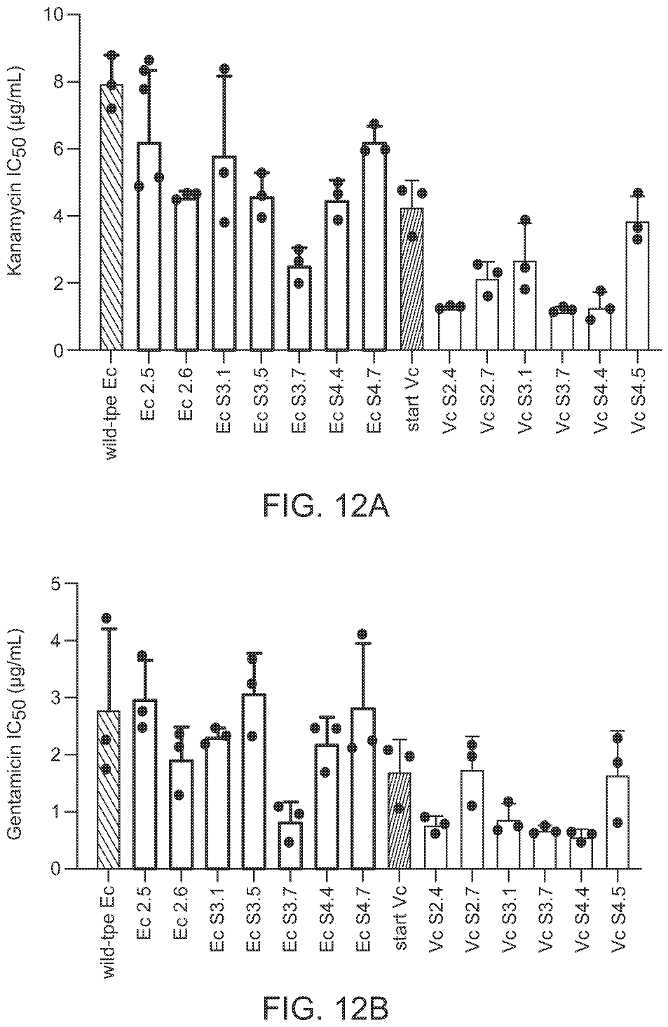

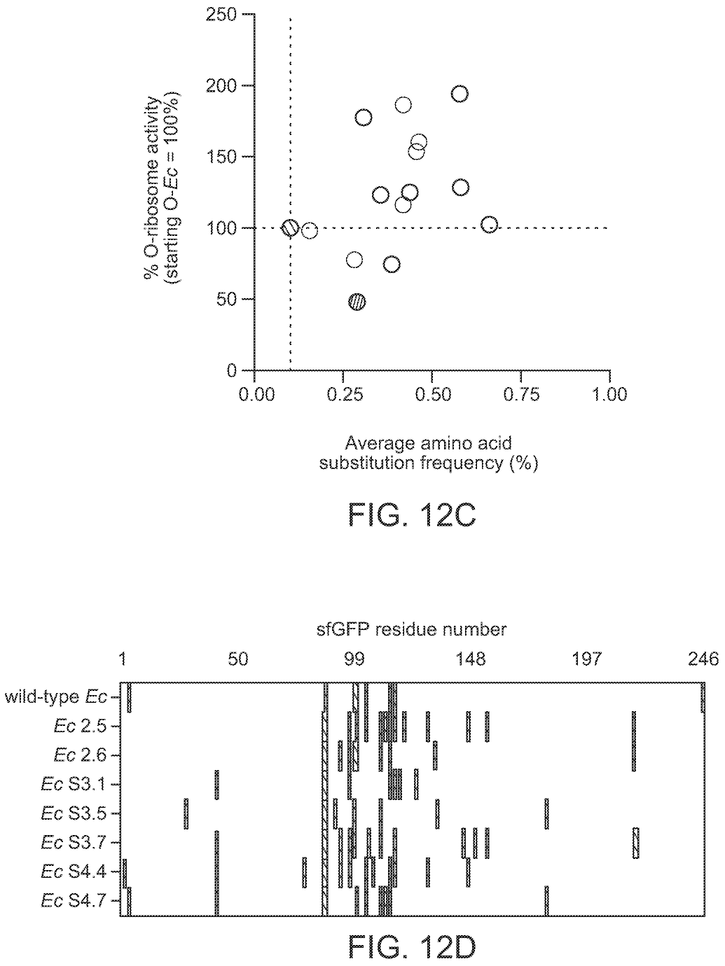

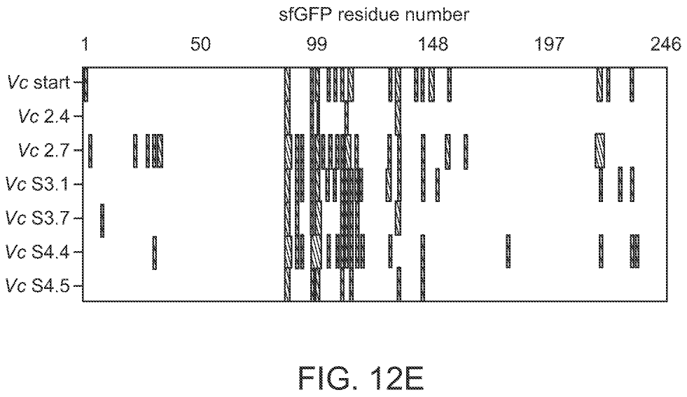

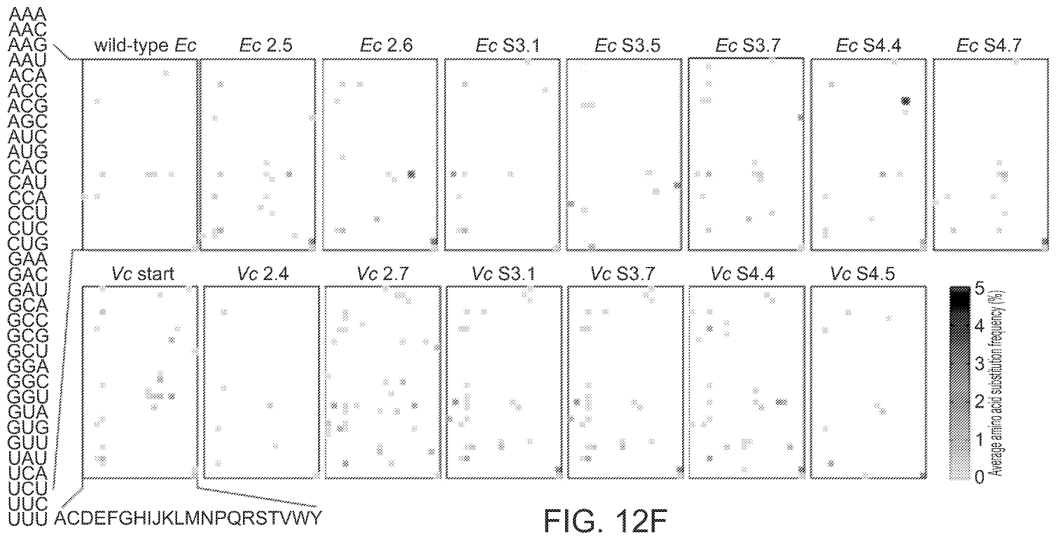

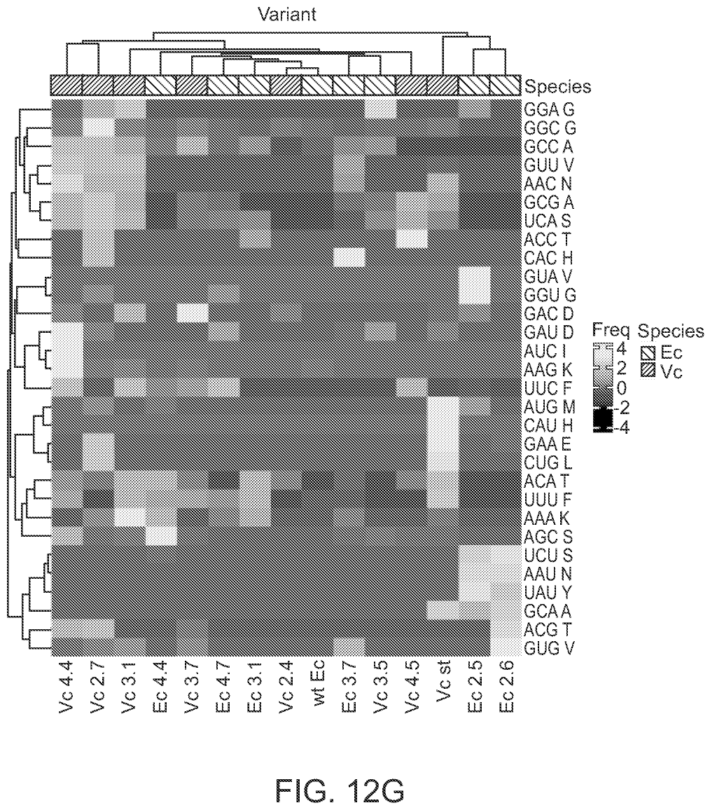

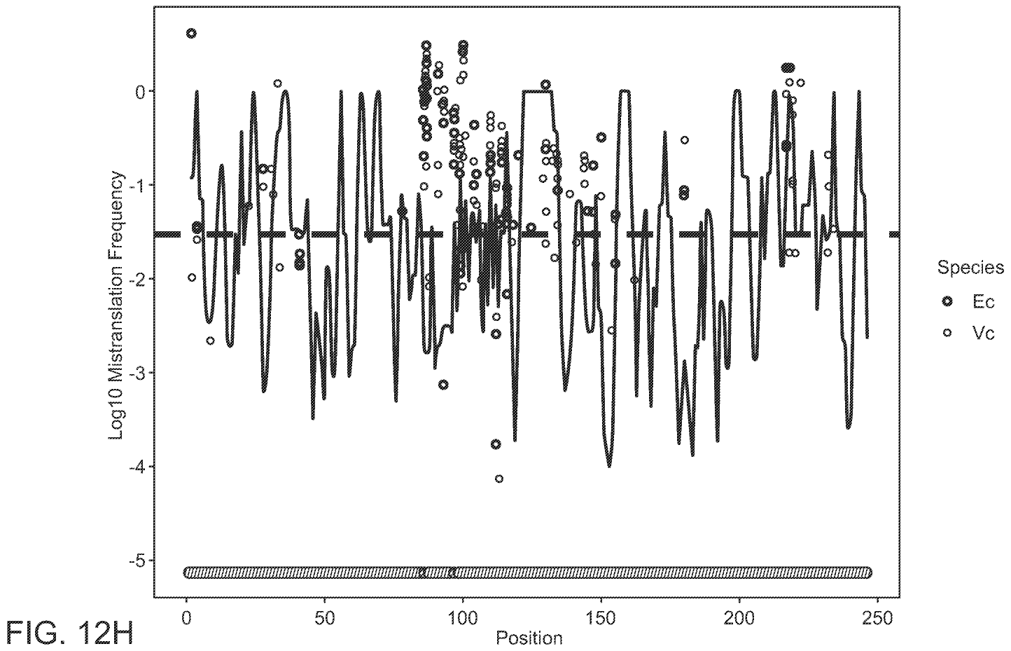

It was hypothesized that the observed reduction in membrane integrity and cell population growth may derive from protein mistranslation by evolved rRNAs. Whereas perturbation of translation rates through ribosomal protein (rpsD, rpsE) mutations can impact the fidelity of protein synthesis (Bjorkman et al., 1999), no such relationship between speed and fidelity appears to have been previously identified for kinetically-enhanced translation. To explore this relationship, complemented SQ171 strains were tested for sensitivity to aminoglycosides as a marker of amino acid mis-incorporation (Recht and Puglisi, 2001). Interestingly, it was identified that sensitivity to the aminoglycosides kanamycin and gentamicin correlated negatively with E. coli-(Pearson Correlation Coefficient, or PCC=−0.6086) and V. cholerae-derived variants (PCC=−0.5248) (FIGS. 11G, 11H, FIGS. 12A, 12B). SQ171 strains encoding evolved rRNAs also showed an increase in overall cell volume that negatively correlated with population doubling time (FIG. 11I), which indicated that kinetically-enhanced ribosomes may impact cell size by accumulating mistranslated proteins at a non-physiological rate, thereby impacting the balance between cell growth and division (Basan et al., 2015). It was noted, however, that increased cell volume under nutrient-rich growth conditions is correlated with higher average cell growth rate (Taheri-Araghi et al., 2015), yet this relationship was absent for the currently disclosed kinetically evolved rRNA variants.

Motivated by these observations, the translational fidelity of evolved rRNAs was investigated. Complemented SQ171 strain-derived sfGFP was subjected to trypsinization and label-free LC-MS/MS to quantify amino acid mis-incorporation (substitution) events (FIG. 11J) (Mordret et al., 2019). For strains encoding wild-type E. coli and the starting V. cholerae strain rRNAs, a median amino acid substitution frequency was observed between 1×10⁻³-10⁻⁴, which indicated that these ribosomes translate with natural tolerable error rates (FIG. 11K). All E. coli-derived and 4/6 tested V. cholerae-derived mutants, displayed median amino acid substitution frequencies above ≥2×10⁻³ (FIG. 6K). O-ribosome activity measured through kinetic luminescence monitoring correlated positively with amino acid substitution frequency (FIG. 12C), which indicated a universal selection outcome where enhanced translation kinetics was concomitant with increased mistranslation. Interestingly, specific regions of the sfGFP transcript were enriched in mistranslation events (FIG. 12D, 12E), although no clear codon ambiguity or amino acid mistranslation preference emerged from these analyses (FIG. 12F).

In general, PACE-evolved o-rRNA variants showed enhanced translation rate over starting E. coli rRNA under various reporter gene and o-RBS contexts (FIGS. 9A-9J). To investigate if these observations extended to proteome-wide translation, the relative translation rates of complemented SQ171 strains were quantified through detection of L-azidohomoalanine (AHA) (FIG. 11L) incorporation in defined synthetic M9 minimal medium and quantification following click-chemistry labeling (Hatzenpichler et al., 2014). Higher average translation rates in V. cholera mutants (S2.7, S3.7) and E. coli mutants (S3.5, S3.7) were observed as compared to E. coli and V. cholera starting ribosomes (FIG. 11M). Analysis of viable cells revealed an average AHA incorporation rate increase of >2-fold by Vc mutants S2.7 and S3.7. Under these amino acid starvation conditions, comparable degrees of reductase activity were observed between all strains (FIG. 11N) and slightly decreased membrane integrity was observed in the Vc mutant S3.7 and S4.4 strains (FIG. 11O). Overall, these data showcase the ability of the discovered mutations to impart enhanced kinetic properties to ribosomes in nearly native settings, and that faster translation results in a concomitant reduction in translational fidelity. These data indicate that ribosome kinetic potential is not maximized but rather refined within a cellular context to balance translation rate and error.

Established models intimately link bacterial ribosome content, proteome-wide protein synthesis rate and population proliferation rate (Scott et al., 2014). Whereas reduction of ribosome elongation rate can negatively impact bacterial proliferation rates (Vallabhaneni and Farabaugh, 2009), it has previously been unclear if kinetically-enhanced ribosomes would result in a correspondingly rapid population growth. Curiously, reduced translation kinetics can enhance the fidelity of protein synthesis (Riba et al., 2019), which has indicated that some interplay between these two parameters could exist. The instant disclosure remarkably has provided a successful outcome in an attempt to enhance ribosome translation rates above natural speeds.

To explore the relationship between kinetics and cell proliferation, it was envisioned at the outset of the experiments disclosed herein that ribosome directed evolution would provide access to genotypes with faster-than-natural translation rates. To overcome inherent challenges in ribosome directed evolution, the o-Ribo-PACE process of the instant disclosure was developed, which combines in vivo orthogonal translation (Aleksashin et al., 2019) and phage-assisted continuous evolution (Esvelt, Carlson and Liu, 2011). To afford this system, the following parameters of the platform known to affect the efficiency of orthogonal translation were systematically optimized: 1) o-RBS/o-antiRBS interactions to limit crosstalk with host ribosomes, 2) sensor plasmid architecture to enhance orthogonal translation sensitivity, and 3) deletion of host hibernation factors that were show herein, for the first time, to be capable of limiting orthogonal translation capabilities. These advances yielded a new orthogonal translation system that supported phage propagation with high efficiency and minimal crosstalk (>70,000-fold above background). This system was then validated using orthogonal Escherichia coli-derived ribosomes, and these capabilities were also extended to two related heterologous ribosomes from P. aeruginosa and V. cholerae (Kolber et al., 2021).

Convergent two and three-stage o-Ribo-PACE selection regimes yielded o-rRNA variants possessing putatively enhanced kinetic activity above starting rRNA scaffolds. Representative variants were validated using multiple reporter genes, o-RBS/antiRBS pairs, and r-proteins complements, and context-independent improvements in protein translation were identified. Interestingly, consensus mutations were discovered at positions known to interact with ribosomal proteins uS2, uS4, and uS12, all of which play roles in tRNA selection (FIGS. 7A-7F) (Vila-Sanjurjo et al., 2003; Alksne et al., 1993; Zaher and Green, 2010), which indicated general solutions for rapid translation using diverse o-rRNA scaffolds. However, some discrepancies between activities of evolved heterologous o-rRNA variants were also noted that cannot be exclusively attributed to phylogenetic distance from E. coli, but may reflect incompatibility between heterologous components and the host translational machinery. In some cases, PACE-derived variants had contrasting effects in orthogonal translation and SQ171 complementation assays, which indicated that requirements for these activities may not be fully congruent.

Using these evolved ribosomes, proteome-wide translation rate was observed to have increased by >2-fold (FIG. 11M) without a corresponding increase in population growth rate. In addition, strains encoding kinetically-enhanced ribosomes showed an elevated fraction of non-viable cells and increased sensitivity to aminoglycosides, implicating mistranslation of host proteins as a bystander effect of faster translation. In vitro analysis of purified protein from SQ171 strains confirmed this hypothesis, and showed significant increases in amino acid substitution frequency (FIG. 11K). It is worth highlighting that E. coli and V. cholerae-derived rRNAs shared consensus mutations in the A-site of the 16S rRNA, and demonstrated a propensity for amino acid substitutions through codon mistranslation. These observations indicate a general mechanism for overcoming kinetic selection through codon-anticodon ambiguity.

The findings of the instant disclosure therefore showcase that faster-than-natural translation rates are permitted in biological context. The variant rRNAs described herein are therefore contemplated for use in improved protein biomanufacturing, as well as in novel explorations of cell growth regulation and of the ribosome's structure-function relationships.

TABLE 1
Selected Sequences of the Disclosure (See also FIGs. 13-15 for sequences)

| | | SEQ |
|---|---|---|
| | | ID |
| Name | Sequence | NO: |
| wt RBS | UUUCCAAGGAGGGAUCUAUG | 1 |
| wt antiRBS | AAUUCCUCCACUA | 2 |
| o-RBSB | UUUCCAACCACAGAUCUAUG | 3 |
| o-antiRBSB | AUUGGUGUACUA | 4 |
| o-RBSlib | UUUCCANNNNNNNGAUCUAUG | 5 |
| o- | AUNNNNNNACUA | 6 |
| antiRBSlib | | |
| o-RBSH3 | UUUCCAAUAUACAGAUCUAUG | 7 |
| o- | AUAUAUGUACUA | 8 |
| antiRBSH3 | | |
| o- | AUAUGUUCACUA | 9 |
| antiRBSH3- | | |
| 1 | | |
| o- | AUUAUGUAACUA | 10 |
| antiRBSH3- | | |
| 2 | | |
| E. coli | GCAGCCATGCCGCGTGTATGAAGAAGGCCTTCGGGTTGTAAAGTACTTTCAGCGGGGAGGA | 11 |
| 16S rRNA | AGGGAGTAA | |
| FIG. 6I | | |
| segment, | | |
| wt | | |
| P. | CCAGCCATGCCGCGTGTGTGAAGAAGGTCTTCGGATTGTAAAGCACTTTAAGTTGGGAGGA | 12 |
| aeruginosa | AGGGCAGTA | |
| 16S rRNA | | |
| FIG. 6I | | |
| segment, | | |
| wt | | |
| P. | CCAGCCATGCCGCGTGTGTGAAGAAGGTCTTCGGATTGTAAAGCACTTTTAGTTGGGAGGA | 13 |
| aeruginosa | AGGGCAGTA | |
| 16S rRNA | | |
| FIG. 6I | | |
| segment, | | |
| A434U | | |
| V. | GCAGCCATGCCGCGTGTATGAAGAAGGCCTTCGGGTTGTAAAGTACTTTCAGTAGGGAGGA | 14 |
| cholerae | AGGTGGTTA | |
| 16S rRNA | | |
| FIG. 6I | | |
| segment, | | |
| wt | | |
| V. | GCAGCCATGCCGCGTGTACGAAGAAGGCCTTCGGGTTGTAAAGTACTTTCAGTAGGGAGGA | 15 |
| cholerae | AGGTGGTTA | |
| 16S rRNA | | |
| FIG. 6I | | |
| segment, | | |
| U409C | | |
| E. coli | TAAGTCGACCGCCTGGGGAGTACGGCCGCAAGGTTAAAACTCAAATGAATTGACGGGGGCC | 16 |
| 16S rRNA | CGCACAAGC | |
| FIG. 6I | | |
| segment, | | |
| wt | | |
| E. coli | TAAGTCGACCGCCTGGGGAGTACGGCCGCAAGGTCAGAACTCAAATGAATTGACGGGGGCC | 17 |
| 16S rRNA | CGCACAAGC | |
| FIG. 6I | | |
| segment, | | |
| U904C, | | |
| A906G | | |
| P. | TAAGTCGACCGCCTGGGGAGTACGGCCGCAAGGTTAAAACTCAAATGAATTGACGGGGGCC | 18 |
| aeruginosa | CGCACAAGC | |
| 16S rRNA | | |
| FIG. 6I | | |
| segment, | | |
| wt | | |
| P. | TAAGTCGACCGCCTGGGGAGTACGGCCGCAAGGTTAGAACTCAAATGAATTGACGGGGGCC | 19 |
| aeruginosa | CGCACAAGC | |
| 16S rRNA | | |
| FIG. 6I | | |
| segment, | | |
| A900G | | |
| V. | TAAGTAGACCGCCTGGGGAGTACGGTCGCAAGATTAAAACTCAAATGAATTGACGGGGGCC | 20 |
| cholerae | CGCACAAGC | |
| 16S rRNA | | |
| FIG. 6I | | |
| segment, | | |
| wt | | |
| V. | TAAGTAGACCGCCTGGGGAGTACGGTCGCAAGATCAGAACTCAAATGAATTGACGGGGGCC | 21 |
| cholerae | CGCACAAGC | |
| 16S rRNA | | |
| FIG. 6I | | |
| segment, | | |
| U904C, | | |
| A906G | | |
| E. coli | GTCGTCAGCTCGTGTTGTGAAATGTTGGGTTAAGTCCCGCAACGAGCGCAACCCTTATCCT | 22 |
| 16S rRNA | TTGTTGCCA | |
| FIG. 6I | | |
| segment, | | |
| wt | | |
| E. coli | GTCGTCAGCTCGTGTTGTGAAATGTTGGGTTAAGTCCTGCAACGAGCGCAACCCTTATCCT | 23 |
| 16S rRNA | TTGTTGCCA | |
| FIG. 6I | | |
| segment, | | |
| C1098U | | |
| P. | GTCGTCAGCTCGTGTCGTGAGATGTTGGGTTAAGTCCCGTAACGAGCGCAACCCTTGTCCT | 24 |
| aeruginosa | TAGTTACCA | |
| 16S rRNA | | |
| FIG. 6I | | |
| segment, | | |
| wt | | |
| V. | GTCGTCAGCTCGTGTTGTGAAATGTTGGGTTAAGTCCCGCAACGAGCGCAACCCTTATCCT | 25 |
| cholerae | TGTTTGCCA | |
| 16S rRNA | | |
| FIG. 6I | | |
| segment, | | |
| wt | | |
| E. coli | TCCCGGGCCTTGTACACACCGCCCGTCACACCATGGGAGTGGGTTGCAAAAGAAGTAGGTA | 26 |
| 16S rRNA | GCTTAACCT | |
| FIG. 6I | | |
| segment, | | |
| wt | | |
| E. coli | TCCCGGGCCTTGTACACACCGCCCGTCACACCATAGGAGTGGGTTGCAAAAGAAGTAGGTA | 27 |
| 16S rRNA | GCTTAACCT | |
| FIG. 6I | | |
| segment, | | |
| G1415A | | |
| P. | TCCCGGGCCTTGTACACACCGCCCGTCACACCATGGGAGTGGGTTGCTCCAGAAGTAGCTA | 28 |
| aeruginosa | GTCTAACCG | |
| 16S rRNA | | |
| FIG. 6I | | |
| segment, | | |
| wt | | |
| V. | TCCCGGGCCTTGTACACACCGCCCGTCACACCATGGGAGTGGGCTGCAAAAGAAGCAGGTA | 29 |
| cholerae | GTTTAACCT | |
| 16S rRNA | | |
| FIG. 6I | | |
| segment, | | |
| wt | | |
| E. coli | TCGGGAGGGCGCTTACCACTTTGTGATTCATGACTGGGGTGAAGTCGTAACAAGGTAACCG | 30 |
| 16S rRNA | TAGGGGAAC | |
| FIG. 6I | | |
| segment, | | |
| wt | | |
| E. coli | TCGGGAGGGCGCTTACCACTTTGTGATTCATGACTGAGGTGAAGTCGTAACAAGGTAACCG | 31 |
| 16S rRNA | TAGGGGAAC | |
| FIG. 6I | | |
| segment, | | |
| G1487A | | |
| P. | CAAGGGGGACGGTTACCACGGAGTGATTCATGACTAGGGTGAAGTCGTAACAAGGTAGCCG | 32 |
| aeruginosa | TAGGGGAAC | |
| 16S rRNA | | |
| FIG. 6I | | |
| segment, | | |
| wt | | |
| V. | TCGGGAGGACGCTTGCCACTTTGTGGTTCATGACTGGGGTGAAGTCGTAACAAGGTAGCGC | 33 |
| cholerae | TAGGGGAAC | |
| 16S rRNA | | |
| FIG. 6I | | |
| segment, | | |
| wt | | |
| V. | TCGGGAGGACGCTTGCCACTTTGTGGTTCATGACTGAGGTGAAGTCGTAACAAGGTAGCGC | 34 |
| cholerae | TAGGGGAAC | |
| 16S rRNA | | |
| FIG. 6I | | |
| segment, | | |
| G1487A | | |
| E. coli | ATCGCTAGTAATCGTGGATCAGAATGCCACGGTGAATACG | 35 |
| 16S rRNA | | |
| FIG. 10F | | |
| segment, | | |
| BsmI site | | |
| P. | ATCGCTAGTAATCGTGAATCAGAATGTCACGGTGAATACG | 36 |
| aeruginosa | | |
| 16S rRNA | | |
| FIG. 10F | | |
| segment, | | |
| BsmI site | | |
| V. | ATCGCTAGTAATCGCAAATCAGAATGTTGCGGTGAATACG | 37 |
| cholerae | | |
| 16S rRNA | | |
| FIG. 10F | | |
| segment, | | |
| BsmI site | | |
| AB5606 | CGGTGGAGCATGTGGTTT | 38 |
| AB5113 | ACGCCTTGCTTTTCACTTTC | 39 |
| E. coli | AAATTGAAGAGTTTGATCATGGCTCAGATTGAACGCTGGCGGCAGGCCTAACACATGCAAG | 40 |
| 16S rRNA, | TCGAACGGTAACAGGAAGAAGCTTGCTTCTTTGCTGACGAGTGGCGGACGGGTGAGTAATG | |
| wt | TCTGGGAAACTGCCTGATGGAGGGGGATAACTACTGGAAACGGTAGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCACCTCCTTA | |
| E. coli | AAATTGAAGAGTTTGATCATGGCTCAGATTGAACGCTGGCGGCAGGCCTAACACATGCAAG | 41 |
| 16S rRNA, | TCGAACGGTAACAGGAAGAAGCTTGCTCTTTGCTGACGAGTGGCGGACGGGTGAGTAATGT | |
| U409C | CTGGGAAACTGCCTGATGGAGGGGGATAACTACTGGAAACGGTAGCTAATACCGCATAACG | |
| | TCGCAAGACCAAAGAGGGGGACCTTCGGGCCTCTTGCCATCGGATGTGCCCAGATGGGATT | |
| | AGCTAGTAGGTGGGGTAACGGCTCACCTAGGCGACGATCCCTAGCTGGTCTGAGAGGATGA | |
| | CCAGCCACACTGGAACTGAGACACGGTCCAGACTCCTACGGGAGGCAGCAGTGGGGAATAT | |
| | TGCACAATGGGCGCAAGCCTGATGCAGCCATGCCGCGTGTACGAAGAAGGCCTTCGGGTTG | |
| | TAAAGTACTTTCAGCGGGGAGGAAGGGAGTAAAGTTAATACCTTTGCTCATTGACGTTACC | |
| | CGCAGAAGAAGCACCGGCTAACTCCGTGCCAGCAGCCGCGGTAATACGGAGGGTGCAAGCG | |
| | TTAATCGGAATTACTGGGCGTAAAGCGCACGCAGGCGGTTTGTTAAGTCAGATGTGAAATC | |
| | CCCGGGCTCAACCTGGGAACTGCATCTGATACTGGCAAGCTTGAGTCTCGTAGAGGGGGGT | |
| | AGAATTCCAGGTGTAGCGGTGAAATGCGTAGAGATCTGGAGGAATACCGGTGGCGAAGGCG | |
| | GCCCCCTGGACGAAGACTGACGCTCAGGTGCGAAAGCGTGGGGAGCAAACAGGATTAGATA | |
| | CCCTGGTAGTCCACGCCGTAAACGATGTCGACTTGGAGGTTGTGCCCTTGAGGCGTGGCTT | |
| | CCGGAGCTAACGCGTTAAGTCGACCGCCTGGGGAGTACGGCCGCAAGGTTAAAACTCAAAT | |
| | GAATTGACGGGGGCCCGCACAAGCGGTGGAGCATGTGGTTTAATTCGATGCAACGCGAAGA | |
| | ACCTTACCTGGTCTTGACATCCACGGAAGTTTTCAGAGATGAGAATGTGCCTTCGGGAACC | |
| | GTGAGACAGGTGCTGCATGGCTGTCGTCAGCTCGTGTTGTGAAATGTTGGGTTAAGTCCCG | |
| | CAACGAGCGCAACCCTTATCCTTTGTTGCCAGCGGTCCGGCCGGGAACTCAAAGGAGACTG | |
| | CCAGTGATAAACTGGAGGAAGGTGGGGATGACGTCAAGTCATCATGGCCCTTACGACCAGG | |
| | GCTACACACGTGCTACAATGGCGCATACAAAGAGAAGCGACCTCGCGAGAGCAAGCGGACC | |
| | TCATAAAGTGCGTCGTAGTCCGGATTGGAGTCTGCAACTCGACTCCATGAAGTCGGAATCG | |
| | CTAGTAATCGTGGATCAGAATGCCACGGTGAATACGTTCCCGGGCCTTGTACACACCGCCC | |
| | GTCACACCATGGGAGTGGGTTGCAAAAGAAGTAGGTAGCTTAACCTTCGGGAGGGCGCTTA | |
| | CCACTTTGTGATTCATGACTGGGGTGAAGTCGTAACAAGGTAACCGTAGGGGAACCTGCGG | |
| | TTGGATCACCTCCTTA | |
| E. coli | AAATTGAAGAGTTTGATCATGGCTCAGATTGAACGCTGGCGGCAGGCCTAACACATGCAAG | 42 |
| 16S rRNA, | TCGAACGGTAACAGGAAGAAGCTTGCTCTTTGCTGACGAGTGGCGGACGGGTGAGTAATGT | |
| G1487A | CTGGGAAACTGCCTGATGGAGGGGGATAACTACTGGAAACGGTAGCTAATACCGCATAACG | |
| | TCGCAAGACCAAAGAGGGGGACCTTCGGGCCTCTTGCCATCGGATGTGCCCAGATGGGATT | |
| | AGCTAGTAGGTGGGGTAACGGCTCACCTAGGCGACGATCCCTAGCTGGTCTGAGAGGATGA | |
| | CCAGCCACACTGGAACTGAGACACGGTCCAGACTCCTACGGGAGGCAGCAGTGGGGAATAT | |
| | TGCACAATGGGCGCAAGCCTGATGCAGCCATGCCGCGTGTATGAAGAAGGCCTTCGGGTTG | |
| | TAAAGTACTTTCAGCGGGGAGGAAGGGAGTAAAGTTAATACCTTTGCTCATTGACGTTACC | |
| | CGCAGAAGAAGCACCGGCTAACTCCGTGCCAGCAGCCGCGGTAATACGGAGGGTGCAAGCG | |
| | TTAATCGGAATTACTGGGCGTAAAGCGCACGCAGGCGGTTTGTTAAGTCAGATGTGAAATC | |
| | CCCGGGCTCAACCTGGGAACTGCATCTGATACTGGCAAGCTTGAGTCTCGTAGAGGGGGGT | |
| | AGAATTCCAGGTGTAGCGGTGAAATGCGTAGAGATCTGGAGGAATACCGGTGGCGAAGGCG | |
| | GCCCCCTGGACGAAGACTGACGCTCAGGTGCGAAAGCGTGGGGAGCAAACAGGATTAGATA | |
| | CCCTGGTAGTCCACGCCGTAAACGATGTCGACTTGGAGGTTGTGCCCTTGAGGCGTGGCTT | |
| | CCGGAGCTAACGCGTTAAGTCGACCGCCTGGGGAGTACGGCCGCAAGGTTAAAACTCAAAT | |
| | GAATTGACGGGGGCCCGCACAAGCGGTGGAGCATGTGGTTTAATTCGATGCAACGCGAAGA | |
| | ACCTTACCTGGTCTTGACATCCACGGAAGTTTTCAGAGATGAGAATGTGCCTTCGGGAACC | |
| | GTGAGACAGGTGCTGCATGGCTGTCGTCAGCTCGTGTTGTGAAATGTTGGGTTAAGTCCCG | |
| | CAACGAGCGCAACCCTTATCCTTTGTTGCCAGCGGTCCGGCCGGGAACTCAAAGGAGACTG | |
| | CCAGTGATAAACTGGAGGAAGGTGGGGATGACGTCAAGTCATCATGGCCCTTACGACCAGG | |
| | GCTACACACGTGCTACAATGGCGCATACAAAGAGAAGCGACCTCGCGAGAGCAAGCGGACC | |
| | TCATAAAGTGCGTCGTAGTCCGGATTGGAGTCTGCAACTCGACTCCATGAAGTCGGAATCG | |
| | CTAGTAATCGTGGATCAGAATGCCACGGTGAATACGTTCCCGGGCCTTGTACACACCGCCC | |
| | GTCACACCATGGGAGTGGGTTGCAAAAGAAGTAGGTAGCTTAACCTTCGGGAGGGCGCTTA | |
| | CCACTTTGTGATTCATGACTAGGGTGAAGTCGTAACAAGGTAACCGTAGGGGAACCTGCGG | |
| | TTGGATCACCTCCTTA | |
| E. coli | AAATTGAAGAGTTTGATCATGGCTCAGATTGAACGCTGGCGGCAGGCCTAACACATGCAAG | 43 |
| 16S rRNA, | TCGAACGGTAACAGGAAGAAGCTTGCTCTTTGCTGACGAGTGGCGGACGGGTGAGTAATGT | |
| U409C, | CTGGGAAACTGCCTGATGGAGGGGGATAACTACTGGAAACGGTAGCTAATACCGCATAACG | |
| G1487A | TCGCAAGACCAAAGAGGGGGACCTTCGGGCCTCTTGCCATCGGATGTGCCCAGATGGGATT | |
| | AGCTAGTAGGTGGGGTAACGGCTCACCTAGGCGACGATCCCTAGCTGGTCTGAGAGGATGA | |
| | CCAGCCACACTGGAACTGAGACACGGTCCAGACTCCTACGGGAGGCAGCAGTGGGGAATAT | |
| | TGCACAATGGGCGCAAGCCTGATGCAGCCATGCCGCGTGTACGAAGAAGGCCTTCGGGTTG | |
| | TAAAGTACTTTCAGCGGGGAGGAAGGGAGTAAAGTTAATACCTTTGCTCATTGACGTTACC | |
| | CGCAGAAGAAGCACCGGCTAACTCCGTGCCAGCAGCCGCGGTAATACGGAGGGTGCAAGCG | |
| | TTAATCGGAATTACTGGGCGTAAAGCGCACGCAGGCGGTTTGTTAAGTCAGATGTGAAATC | |
| | CCCGGGCTCAACCTGGGAACTGCATCTGATACTGGCAAGCTTGAGTCTCGTAGAGGGGGGT | |
| | AGAATTCCAGGTGTAGCGGTGAAATGCGTAGAGATCTGGAGGAATACCGGTGGCGAAGGCG | |
| | GCCCCCTGGACGAAGACTGACGCTCAGGTGCGAAAGCGTGGGGAGCAAACAGGATTAGATA | |
| | CCCTGGTAGTCCACGCCGTAAACGATGTCGACTTGGAGGTTGTGCCCTTGAGGCGTGGCTT | |
| | CCGGAGCTAACGCGTTAAGTCGACCGCCTGGGGAGTACGGCCGCAAGGTTAAAACTCAAAT | |
| | GAATTGACGGGGGCCCGCACAAGCGGTGGAGCATGTGGTTTAATTCGATGCAACGCGAAGA | |
| | ACCTTACCTGGTCTTGACATCCACGGAAGTTTTCAGAGATGAGAATGTGCCTTCGGGAACC | |
| | GTGAGACAGGTGCTGCATGGCTGTCGTCAGCTCGTGTTGTGAAATGTTGGGTTAAGTCCCG | |
| | CAACGAGCGCAACCCTTATCCTTTGTTGCCAGCGGTCCGGCCGGGAACTCAAAGGAGACTG | |
| | CCAGTGATAAACTGGAGGAAGGTGGGGATGACGTCAAGTCATCATGGCCCTTACGACCAGG | |
| | GCTACACACGTGCTACAATGGCGCATACAAAGAGAAGCGACCTCGCGAGAGCAAGCGGACC | |
| | TCATAAAGTGCGTCGTAGTCCGGATTGGAGTCTGCAACTCGACTCCATGAAGTCGGAATCG | |
| | CTAGTAATCGTGGATCAGAATGCCACGGTGAATACGTTCCCGGGCCTTGTACACACCGCCC | |
| | GTCACACCATGGGAGTGGGTTGCAAAAGAAGTAGGTAGCTTAACCTTCGGGAGGGCGCTTA | |
| | CCACTTTGTGATTCATGACTAGGGTGAAGTCGTAACAAGGTAACCGTAGGGGAACCTGCGG | |
| | TTGGATCACCTCCTTA | |
| S. | AAATTGAAGAGTTTGATCATGGCTCAGATTGAACGCTGGCGGCAGGCCTAACACATGCAAG | 44 |
| enterica | TCGAACGGTAACAGGAAGCAGCTTGCTGCTTCGCTGACGAGTGGCGGACGGGTGAGTAATG | |
| 16S rRNA, | TCTGGGAAACTGCCTGATGGAGGGGGATAACTACTGGAAACGGTGGCTAATACCGCATAAC | |
| U409C, | GTCGCAAGACCAAAGAGGGGGACCTTCGGGCCTCTTGCCATCAGATGTGCCCAGATGGGAT | |
| G1487A | TAGCTAGTTGGTGAGGTAACGGCTCACCAAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTACGAAGAAGGCCTTCGGGTT | |
| | GTAAAGTACTTTCAGCGGGGAGGAAGGTGTTGTGGTTAATAACCGCAGCAATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTCTGTCAAGTCGGATGTGAAAT | |
| | CCCCGGGCTCAACCTGGGAACTGCATTCGAAACTGGCAGGCTTGAGTCTTGTAGAGGGGGG | |
| | TAGAATTCCAGGTGTAGCGGTGAAATGCGTAGAGATCTGGAGGAATACCGGTGGCGAAGGC | |
| | GGCCCCCTGGACAAAGACTGACGCTCAGGTGCGAAAGCGTGGGGAGCAAACAGGATTAGAT | |
| | ACCCTGGTAGTCCACGCCGTAAACGATGTCTACTTGGAGGTTGTGCCCTTGAGGCGTGGCT | |
| | TCCGGAGCTAACGCGTTAAGTAGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACAGAACTTTCCAGAGATGGATTGGTGCCTTCGGGAAC | |
| | TGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGATTAGGTCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTAGGGTGAAGTCGTAACAAGGTAACCGTAGGGGAACCTGCG | |
| | GTTGGATCATGTGGTTA | |
| C. | AAATTGAAGAGTTTGATCATGGCTCAGATTGAACGCTGGCGGCAGGCCTAACACATGCAAG | 45 |
| freundii | TCGAACGGTAGCACAGAGAGCTTGCTCTCGGGTGACGAGTGGCGGACGGGTGAGTAATGTC | |
| 16S rRNA, | TGGGAAACTGCCCGATGGAGGGGGATAACTACTGGAAACGGTAGCTAATACCGCATAACGT | |
| U409C, | CGCAAGACCAAAGAGGGGGACCTTCGGGCCTCTTGCCATCGGATGTGCCCAGATGGGATTA | |
| G1487A | GCTAGTAGGTGGGGTAACGGCTCACCTAGGCGACGATCCCTAGCTGGTCTGAGAGGATGAC | |
| | CAGCCACACTGGAACTGAGACACGGTCCAGACTCCTACGGGAGGCAGCAGTGGGGAATATT | |
| | GCACAATGGGCGCAAGCCTGATGCAGCCATGCCGCGTGTAcGAAGAAGGCCTTCGGGTTGT | |
| | AAAGTACTTTCAGCGAGGAGGAAGGCGTTGTGGTTAATAACCGCAACGATTGACGTTACTC | |
| | GCAGAAGAAGCACCGGCTAACTCCGTGCCAGCAGCCGCGGTAATACGGAGGGTGCAAGCGT | |
| | TAATCGGAATTACTGGGCGTAAAGCGCACGCAGGCGGTCTGTCAAGTCGGATGTGAAATCC | |
| | CCGGGCTCAACCTGGGAACTGCATCCGAAACTGGCAGGCTAGAGTCTTGTAGAGGGGGGTA | |
| | GAATTCCAGGTGTAGCGGTGAAATGCGTAGAGATCTGGAGGAATACCGGTGGCGAAGGCGG | |
| | CCCCCTGGACAAAGACTGACGCTCAGGTGCGAAAGCGTGGGGAGCAAACAGGATTAGATAC | |
| | CCTGGTAGTCCACGCCGTAAACGATGTCGACTTGGAGGTTGTGCCCTTGAGGCGTGGCTTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTACTCTTGACATCCAGAGAACTTAGCAGAGATGCTTTGGTGCCTTCGGGAACTC | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGGTCCGGCCGGGAACTCAAAGGAGACTGC | |
| | CAGTGATAAACTGGAGGAAGGTGGGGATGACGTCAAGTCATCATGGCCCTTACGAGTAGGG | |
| | CTACACACGTGCTACAATGGCATATACAAAGAGAAGCGACCTCGCGAGAGCAAGCGGACCT | |
| | CATAAAGTATGTCGTAGTCCGGATTGGAGTCTGCAACTCGACTCCATGAAGTCGGAATCGC | |
| | TAGTAATCGTGGATCAGAATGCCACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATTCATGACTGaGGTGAAGTCGTAACAAGGTAACCGTAGGGGAACCTGCGGT | |
| | TGGATCATGTGGTTA | |
| K. | AAATTGAAGAGTTTGATCATGGCTCAGATTGAACGCTGGCGGCAGGCCTAACACATGCAAG | 46 |
| aerogenes | TCGAGCGGTAGCACAGAGAGCTTGCTCTCGGGTGACGAGCGGCGGACGGGTGAGTAATGTC | |
| 16S rRNA, | TGGGAAACTGCCTGATGGAGGGGGATAACTACTGGAAACGGTAGCTAATACCGCATAACGT | |
| U409C, | CGCAAGACCAAAGTGGGGGACCTTCGGGCCTCATGCCATCAGATGTGCCCAGATGGGATTA | |
| G1487A | GCTAGTAGGTGGGGTAATGGCTCACCTAGGCGACGATCCCTAGCTGGTCTGAGAGGATGAC | |
| | CAGCCACACTGGAACTGAGACACGGTCCAGACTCCTACGGGAGGCAGCAGTGGGGAATATT | |
| | GCACAATGGGCGCAAGCCTGATGCAGCCATGCCGCGTGTAcGAAGAAGGCCTTCGGGTTGT | |
| | AAAGTACTTTCAGCGAGGAGGAAGGCGTTAAGGTTAATAACCTTGGCGATTGACGTTACTC | |
| | GCAGAAGAAGCACCGGCTAACTCCGTGCCAGCAGCCGCGGTAATACGGAGGGTGCAAGCGT | |
| | TAATCGGAATTACTGGGCGTAAAGCGCACGCAGGCGGTCTGTCAAGTCGGATGTGAAATCC | |
| | CCGGGCTCAACCTGGGAACTGCATTCGAAACTGGCAGGCTAGAGTCTTGTAGAGGGGGGTA | |
| | GAATTCCAGGTGTAGCGGTGAAATGCGTAGAGATCTGGAGGAATACCGGTGGCGAAGGCGG | |
| | CCCCCTGGACAAAGACTGACGCTCAGGTGCGAAAGCGTGGGGAGCAAACAGGATTAGATAC | |
| | CCTGGTAGTCCACGCCGTAAACGATGTCGACTTGGAGGTTGTGCCCTTGAGGCGTGGCTTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTACTCTTGACATCCAGAGAACTTAGCAGAGATGCTTTGGTGCCTTCGGGAACTC | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGATTCGGTCGGGAACTCAAAGGAGACTGC | |
| | CAGTGATAAACTGGAGGAAGGTGGGGATGACGTCAAGTCATCATGGCCCTTACGAGTAGGG | |
| | CTACACACGTGCTACAATGGCATATACAAAGAGAAGCGACCTCGCGAGAGCAAGCGGACCT | |
| | CATAAAGTATGTCGTAGTCCGGATTGGAGTCTGCAACTCGACTCCATGAAGTCGGAATCGC | |
| | TAGTAATCGTAGATCAGAATGCTACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATTCATGACTGaGGTGAAGTCGTAACAAGGTAACCGTAGGGGAACCTGCGGT | |
| | TGGATCAtgtggtTA | |
| K. | AAATTGAAGAGTTTGATCATGGCTCAGATTGAACGCTGGCGGCAGGCCTAACACATGCAAG | 47 |
| pneumoniae | TCGAGCGGTAGCACAGAGAGCTTGCTCTCGGGTGACGAGCGGCGGACGGGTGAGTAATGTC | |
| 16S rRNA, | TGGGAAACTGCCTGATGGAGGGGGATAACTACTGGAAACGGTAGCTAATACCGCATAACGT | |
| U409C, | CGCAAGACCAAAGTGGGGGACCTTCGGGCCTCATGCCATCAGATGTGCCCAGATGGGATTA | |
| G1487A | GCTAGTAGGTGGGGTAACGGCTCACCTAGGCGACGATCCCTAGCTGGTCTGAGAGGATGAC | |
| | CAGCCACACTGGAACTGAGACACGGTCCAGACTCCTACGGGAGGCAGCAGTGGGGAATATT | |
| | GCACAATGGGCGCAAGCCTGATGCAGCCATGCCGCGTGTGcGAAGAAGGCCTTCGGGTTGT | |
| | AAAGCACTTTCAGCGGGGAGGAAGGCGGTGAGGTTAATAACCTCATCGATTGACGTTACCC | |
| | GCAGAAGAAGCACCGGCTAACTCCGTGCCAGCAGCCGCGGTAATACGGAGGGTGCAAGCGT | |
| | TAATCGGAATTACTGGGCGTAAAGCGCACGCAGGCGGTCTGTCAAGTCGGATGTGAAATCC | |
| | CCGGGCTCAACCTGGGAACTGCATTCGAAACTGGCAGGCTAGAGTCTTGTAGAGGGGGGTA | |
| | GAATTCCAGGTGTAGCGGTGAAATGCGTAGAGATCTGGAGGAATACCGGTGGCGAAGGCGG | |
| | CCCCCTGGACAAAGACTGACGCTCAGGTGCGAAAGCGTGGGGAGCAAACAGGATTAGATAC | |
| | CCTGGTAGTCCACGCCGTAAACGATGTCGATTTGGAGGTTGTGCCCTTGAGGCGTGGCTTC | |
| | CGGAGCTAACGCGTTAAATCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTGGTCTTGACATCCACAGAACTTTCCAGAGATGGATTGGTGCCTTCGGGAACTG | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGGTTAGGCCGGGAACTCAAAGGAGACTGC | |
| | CAGTGATAAACTGGAGGAAGGTGGGGATGACGTCAAGTCATCATGGCCCTTACGACCAGGG | |
| | CTACACACGTGCTACAATGGCATATACAAAGAGAAGCGACCTCGCGAGAGCAAGCGGACCT | |
| | CATAAAGTATGTCGTAGTCCGGATTGGAGTCTGCAACTCGACTCCATGAAGTCGGAATCGC | |
| | TAGTAATCGTAGATCAGAATGCTACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATTCATGACTGaGGTGAAGTCGTAACAAGGTAACCGTAGGGGAACCTGCGGT | |
| | TGGATCATGTGGTTA | |
| K. oxytoca | AAATTGAAGAGTTTGATCATGGCTCAGATTGAACGCTGGCGGCAGGCCTAACACATGCAAG | 48 |
| 16S rRNA, | TCGAACGGTAGCACAGAGAGCTTGCTCTCGGGTGACGAGTGGCGGACGGGTGAGTAATGTC | |
| U409C, | TGGGAAACTGCCCGATGGAGGGGGATAACTACTGGAAACGGTAGCTAATACCGCATAACGT | |
| G1487A | CGCAAGACCAAAGAGGGGGACCTTCGGGCCTCTTGCCATCGGATGTGCCCAGATGGGATTA | |
| | GCTTGTAGGTGAGGTAACGGCTCACCTAGGCGACGATCCCTAGCTGGTCTGAGAGGATGAC | |
| | CAGCCACACTGGAACTGAGACACGGTCCAGACTCCTACGGGAGGCAGCAGTGGGGAATATT | |
| | GCACAATGGGCGCAAGCCTGATGCAGCCATGCCGCGTGTAcGAAGAAGGCCTTCGGGTTGT | |
| | AAAGTACTTTCAGCGGGGAGGAAGGGAGTGAGGTTAATAACCTCATTCATTGACGTTACCC | |
| | GCAGAAGAAGCACCGGCTAACTCCGTGCCAGCAGCCGCGGTAATACGGAGGGTGCAAGCGT | |
| | TAATCGGAATTACTGGGCGTAAAGCGCACGCAGGCGGTCTGTCAAGTCGGATGTGAAATCC | |
| | CCGGGCTCAACCTGGGAACTGCATTCGAAACTGGCAGGCTGGAGTCTTGTAGAGGGGGGTA | |
| | GAATTCCAGGTGTAGCGGTGAAATGCGTAGAGATCTGGAGGAATACCGGTGGCGAAGGCGG | |
| | CCCCCTGGACAAAGACTGACGCTCAGGTGCGAAAGCGTGGGGAGCAAACAGGATTAGATAC | |
| | CCTGGTAGTCCACGCTGTAAACGATGTCGACTTGGAGGTTGTTCCCTTGAGGAGTGGCTTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTACTCTTGACATCCAGAGAACTTAGCAGAGATGCTTTGGTGCCTTCGGGAACTC | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGATTCGGTCGGGAACTCAAAGGAGACTGC | |
| | CAGTGATAAACTGGAGGAAGGTGGGGATGACGTCAAGTCATCATGGCCCTTACGAGTAGGG | |
| | CTACACACGTGCTACAATGGCATATACAAAGAGAAGCGACCTCGCGAGAGCAAGCGGACCT | |
| | CATAAAGTATGTCGTAGTCCGGATTGGAGTCTGCAACTCGACTCCATGAAGTCGGAATCGC | |
| | TAGTAATCGTGGATCAGAATGCCACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATTCATGACTGaGGTGAAGTCGTAACAAGGTAACCGTAGGGGAACCTGCGGT | |
| | TGGATCATGTGGTTA | |
| E. cloacae | AAATTGAAGAGTTTGATCATGGCTCAGATTGAACGCTGGCGGCAGGCCTAACACATGCAAG | 49 |
| 16S rRNA, | TCGAACGGTAACAGGAAGCAGCTTGCTGCTTCGCTGACGAGTGGCGGACGGGTGAGTAATG | |
| U409C, | TCTGGGAAACTGCCTGATGGAGGGGGATAACTACTGGAAACGGTAGCTAATACCGCATAAC | |
| G1487A | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTAcGAAGAAGGCCTTCGGGTT | |
| | GTAAAGTACTTTCAGCGGGGAGGAAGGCGATAAGGTTAATAACCTTGTCGATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTCTGTCAAGTCGGATGTGAAAT | |
| | CCCCGGGCTCAACCTGGGAACTGCATTCGAAACTGGCAGGCTAGAGTCTTGTAGAGGGGGG | |
| | TAGAATTCCAGGTGTAGCGGTGAAATGCGTAGAGATCTGGAGGAATACCGGTGGCGAAGGC | |
| | GGCCCCCTGGACAAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAACTTAGCAGAGATGCTTTGGTGCCTTCGGGAAC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGAGTAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGaGGTGAAGTCGTAACAAGGTAACCGTAGGGGAACCTGCG | |
| | GTTGGATCAtgtggtTA | |
| S. | TAATTGAAGAGTTTGATCATGGCTCAGATTGAACGCTGGCGGCAGGCTTAACACATGCAAG | 50 |
| marcescens | TCGAGCGGTAGCACAGGGGAGCTTGCTCCCTGGGTGACGAGCGGCGGACGGGTGAGTAATG | |
| 16S rRNA, | TCTGGGAAACTGCCTGATGGAGGGGGATAACTACTGGAAACGGTAGCTAATACCGCATAAC | |
| U409C, | GTCGCAAGACCAAAGAGGGGGACCTTCGGGCCTCTTGCCATCAGATGTGCCCAGATGGGAT | |
| G1487A | TAGCTAGTAGGTGGGGTAATGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTGCGAAGAAGGCCTTCGGGTT | |
| | GTAAAGCACTTTCAGCGAGGAGGAAGGTGGTGAGCTTAATACGCTCATCAATTGACGTTAC | |
| | TCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATTTGAAACTGGCAAGCTAGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCTGTAAACGATGTCGATTTGGAGGTTGTGCCCTTGAGGCGTGGCT | |
| | TCCGGAGCTAACGCGTTAAATCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAACTTAGCAGAGATGCTTTGGTGCCTTCGGGAAC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTTCGGCCGGGAACTCAAAGGAGACT | |
| | GCCAGTGATAAACTGGAGGAAGGTGGGGATGACGTCAAGTCATCATGGCCCTTACGAGTAG | |
| | GGCTACACACGTGCTACAATGGCATATACAAAGAGAAGCGACCTCGCGAGAGCAAGCGGAC | |
| | CTCATAAAGTATGTCGTAGTCCGGATTGGAGTCTGCAACTCGACTCCATGAAGTCGGAATC | |
| | GCTAGTAATCGTAGATCAGAATGCTACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTAGGGTGAAGTCGTAACAAGGTAACCGTAGGGGAACCTGCG | |
| | GTTGGATCATGTGGTTA | |
| P. | TAATTGAAGAGTTTGATCATGGCTCAGATTGAACGCTGGCGGCAGGCCTAACACATGCAAG | 51 |
| mirabilis | TCGAGCGGTAACAGGGGAAGCTTGCTTCTCGCTGACGAGCGGCGGACGGGTGAGTAATGTA | |
| 16S rRNA, | TGGGGATCTGCCCGATAGAGGGGGATAACTACTGGAAACGGTGGCTAATACCGCATAATCT | |
| U409C, | CTTAGGAGCAAAGCAGGGGACCTTCGGGCCTTGCGCTGTCGGATGAACCCATATGGGATTA | |
| G1487A | GCTAGTAGGTAAGGTAATGGCTTACCTAGGCGACGATCCCTAGCTGGTCTGAGAGGATGAT | |
| | CAGCCACACTGGGACTGAGACACGGCCCAGACTCCTACGGGAGGCAGCAGTGGGGAATATT | |
| | GCACAATGGGCGCAAGCCTGATGCAGCCATGCCGCGTGTAcGAAGAAGGCCCTAGGGTTGT | |
| | AAAGTACTTTCAGTCGGGAGGAAGGCGTTGATGTTAATACCATCAACGATTGACGTTACCG | |
| | ACAGAAGAAGCACCGGCTAACTCCGTGCCAGCAGCCGCGGTAATACGGAGGGTGCAAGCGT | |
| | TAATCGGAATTACTGGGCGTAAAGCGCACGCAGGCGGTTAATTAAGTTAGATGTGAAATCC | |
| | CCGGGCTTAACCTGGGAATGGCATCTAAGACTGGTTAGCTAGAGTCTTGTAGAGGGGGGTA | |
| | GAATTCCATGTGTAGCGGTGAAATGCGTAGAGATGTGGAGGAATACCGGTGGCGAAGGCGG | |
| | CCCCCTGGACAAAGACTGACGCTCAGGTGCGAAAGCGTGGGGAGCAAACAGGATTAGATAC | |
| | CCTGGTAGTCCACGCTGTAAACGATGTCGATTTGGAGGTTGTTCCCTAGAGGAGTGGCTTC | |
| | CGGAGCTAACGCGTTAAATCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTACTCTTGACATCCAGAGAATTTAGCAGAGATGCTTTAGTGCCTTCGGGAACTC | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGATTCGGTCGGGAACTCAAAGGAGACTGC | |
| | CGGTGATAAACCGGAGGAAGGTGGGGATGACGTCAAGTCATCATGGCCCTTACGAGTAGGG | |
| | CTACACACGTGCTACAATGGCGTATACAAAGAGAAGCGACCTCGCGAGAGCAAGCGGAACT | |
| | CATAAAGTACGTCGTAGTCCGGATTGGAGTCTGCAACTCGACTCCATGAAGTCGGAATCGC | |
| | TAGTAATCGTAGATCAGAATGCTACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATCCATGACTGaGGTGAAGTCGTAACAAGGTAACCGTAGGGGAACCTGCGGT | |
| | TGGATCATGTGGTTA | |
| P. | TAATTGAAGAGTTTGATCATGGCTCAGATTGAACGCTGGCGGCAGGCCTAACACATGCAAG | 52 |
| stuartii | TCGAGCGGTAACAGGAGAAAGCTTGCTTTCTTGCTGACGAGCGGCGGACGGGTGAGTAATG | |
| 16S rRNA, | TATGGGGATCTGCCCGATAGAGGGGGATAACTACTGGAAACGGTGGCTAATACCGCATAAT | |
| U409C, | GTCTACGGACCAAAGCAGGGGCTCTTCGGACCTTGCACTATCGGATGAACCCATATGGGAT | |
| G1487A | TAGCTAGTAGGTGGGGTAAAGGCTCACCTAGGCGACGATCTCTAGCTGGTCTGAGAGGATG | |
| | ATCAGCCACACTGGGACTGAGACACGGCCCAGACTCCTACGGGAGGCAGCAGTGGGGAATA | |
| | TTGCACAATGGGCGCAAGCCTGATGCAGCCATGCCGCGTGTAcGAAGAAGGCCTTAGGGTT | |
| | GTAAAGTACTTTCAGCGGGGAGGAAGGTGATAAGGTTAATACCCTTATTAATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTCAATTAAGTCAGATGTGAAAG | |
| | CCCCGAGCTTAACTTGGGAATTGCATCTGAAACTGGTTGGCTAGAGTCTTGTAGAGGGGGG | |
| | TAGAATTCCATGTGTAGCGGTGAAATGCGTAGAGATGTGGAGGAATACCGGTGGCGAAGGC | |
| | GGCCCCCTGGACAAAGACTGACGCTCAGGTGCGAAAGCGTGGGGAGCAAACAGGATTAGAT | |
| | ACCCTGGTAGTCCACGCTGTAAACGATGTCGATTTAGAGGTTGTGGTCTTGAACCGTGGCT | |
| | TCTGGAGCTAACGCGTTAAATCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGCGAATCCTTTAGAGATAGAGGAGTGCCTTCGGGAAC | |
| | GCTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCACGTAATGGTGGGAACTCAAAGGAGAC | |
| | TGCCGGTGATAAACCGGAGGAAGGTGGGGATGACGTCAAGTCATCATGGCCCTTACGAGTA | |
| | GGGCTACACACGTGCTACAATGGCAGATACAAAGAGAAGCGACCTCGCGAGAGCAAGCGGA | |
| | ACTCATAAAGTCTGTCGTAGTCCGGATTGGAGTCTGCAACTCGACTCCATGAAGTCGGAAT | |
| | CGCTAGTAATCGTAGATCAGAATGCTACGGTGAATACGTTCCCGGGCCTTGTACACACCGC | |
| | CCGTCACACCATGGGAGTGGGTTGCAAAAGAAGTAGGTAGCTTAACCTTCGGGAGGGCGCT | |
| | TACCACTTTGTGATTCATGACTGaGGTGAAGTCGTAACAAGGTAACCGTAGGGGAACCTGC | |
| | GGTTGGATCATGTGGTTA | |
| V. | TAATTGAAGAGTTTGATCATGGCTCAGATTGAACGCTGGCGGCAGGCCTAACACATGCAAG | 53 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| U409C, | CTCGCAAGAGCAAAGCAGGGGACCTTCGGGCCTTGCGCTACCGGATATGCCCAGGTGGGAT | |
| G1487A | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTAcGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGaGGTGAAGTCGTAACAAGGTAGCGCTAGGGGAACCTGG | |
| | CGCTGGATCATGTGGTTA | |
| A. | TAATTGAAGAGTTTGATCATGGCTCAGATTGAACGCTGGCGGCAGGCCTAACACATGCAAG | 54 |
| macleodii | TCGAACGGTAACATTTCTAGCTTGCTAGAAGATGACGAGTGGCGGACGGGTGAGTAATGCT | |
| 16S rRNA, | TGGGAACTTGCCTTTGCGAGGGGGATAACAGTTGGAAACGACTGCTAATACCGCATAATGT | |
| U409C, | CTTCGGACCAAACGGGGCTTAGGCTCCGGCGCAAAGAGAGGCCCAAGTGAGATTAGCTAGT | |
| G1487A | TGGTAAGGTAACGGCTTACCAAGGCGACGATCTCTAGCTGTTCTGAGAGGAAGATCAGCCA | |
| | CACTGGGACTGAGACACGGCCCAGACTCCTACGGGAGGCAGCAGTGGGGAATATTGCACAA | |
| | TGGGGGAAACCCTGATGCAGCCATGCCGCGTGTGcGAAGAAGGCCTTCGGGTTGTAAAGCA | |
| | CTTTCAGTTGTGAGGAAAAGTTAGTAGTTAATACCTGCTAGCCGTGACGTTAACAACAGAA | |
| | GAAGCACCGGCTAACTCCGTGCCAGCAGCCGCGGTAATACGGAGGGTGCGAGCGTTAATCG | |
| | GAATTACTGGGCGTAAAGCGCACGCAGGCGGTTTGTTAAGCTAGATGTGAAAGCCCCGGGC | |
| | TCAACCTGGGATGGTCATTTAGAACTGGCAGACTAGAGTCTTGGAGAGGGGAGTGGAATTC | |
| | CAGGTGTAGCGGTGAAATGCGTAGATATCTGGAGGAACATCAGTGGCGAAGGCGACTCCCT | |
| | GGCCAAAGACTGACGCTCATGTGCGAAAGTGTGGGTAGCGAACAGGATTAGATACCCTGGT | |
| | AGTCCACACCGTAAACGCTGTCTACTAGCTGTGTGTGTCTTTAAGACGTGCGTAGCGAAGC | |
| | TAACGCGCTAAGTAGACCGCCTGGGGAGTACGGCCGCAAGGTTAAAACTCAAATGAATTGA | |
| | CGGGGGCCCGCACAAGCGGTGGAGCATGTGGTTTAATTCGATGCAACGCGAAGAACCTTAC | |
| | CTACACTTGACATGCTGAGAAGTTACTAGAGATAGTTTCGTGCCTTCGGGAACTCAGACAC | |
| | AGGTGCTGCATGGCTGTCGTCAGCTCGTGTCGTGAGATGTTGGGTTAAGTCCCGCAACGAG | |
| | CGCAACCCTTGTCCTTAGTTGCCAGCCTTAAGTTGGGCACTCTAAGGAGACTGCCGGTGAC | |
| | AAACCGGAGGAAGGTGGGGACGACGTCAAGTCATCATGGCCCTTACGTGTAGGGCTACACA | |
| | CGTGCTACAATGGCATTTACAGAGGGAAGCGAGACAGTGATGTGGAGCGGACCCCTTAAAG | |
| | AATGTCGTAGTCCGGATTGGAGTCTGCAACTCGACTCCATGAAGTCGGAATCGCTAGTAAT | |
| | CGCAGGTCAGAATACTGCGGTGAATACGTTCCCGGGCCTTGTACACACCGCCCGTCACACC | |
| | ATGGGAGTGGGATGCAAAAGAAGTAGTTAGTCTAACCTTCGGGAGGACGATTACCACTTTG | |
| | TGTTTCATGACTGaGGTGAAGTCGTAACAAGGTAACCCTAGGGGAACCTGGGGTTGGATCA | |
| | TGTGGTTA | |
| M. | AAACTGAAGAGTTTGATCATGGCTCAGATTGAACGCTGGCGGCAGGCCTAACACATGCAAG | 55 |
| minutulum | TCGAGCGGTAACAGGAGAAGCTTGCTTCTTGCTGACGAGCGGCGGACGGGTGAGTAATACT | |
| 16S rRNA, | TGGGTATCTGCCCGATAGTGGGGGACAACACGGGGAAACTCGTGCTAATACCGCATACGTC | |
| U409C, | CTACGGGAGAAAGCAGGCTTAGGCTTGCGCTATCGGATGAGCCCAAGTCGGATTAGCTAGT | |
| G1487A | TGGTGAGGTAAAGGCTCACCAAGGCGACGATCCGTAACTGGTCTGAGAGGATGATCAGTCA | |
| | CACCGGGACTGAGACACGGCCCGGACTCCTACGGGAGGCAGCAGTGGGGAATATTGGACAA | |
| | TGGGGGCAACCCTGATCCAGCCATGCCGCGTGTGcGAAGAAGGCCTTAGGGTTGTAAAGCA | |
| | CTTTCAGTGAGGAGGAAAAGTTAGTGGTTAATACCCACTAGCCGTGACGTTACTCACAGAA | |
| | GAAGCACCGGCAAACTCCGTGCCAGCAGCCGCGGTAATACGGAGGGTGCGAGCGTTAATCG | |
| | GAATTACTGGGCGTAAAGCGCGCGTAGGCGGTTTGTTAAGCCGGTTGTGAAAGCCCTAGGC | |
| | TCAACCTAGGAACTGCACCCGGAACTGGCAAGCTAGAGTACAGTAGAGGAAGGTGGAATTC | |
| | CACGTGTAGCGGTGAAATGCGTAGATATGTGGAGGAACATCAGTGGCGAAGGCGGCCTTCT | |
| | GGACTGATACTGACGCTGAGGTGCGAAAGCGTGGGGAGCAAACAGGATTAGATACCCTGGT | |
| | AGTCCACGCCGTAAACGATGTCAACTAGTTGTTGGAACCCTTGAGGTTTTAGTAACGCAGC | |
| | TAACGCATTAAGTTGACCGCCTGGGGAGTACGGCCGCAAGGTTAAAACTCAAATGAATTGA | |
| | CGGGGGCCCGCACAAGCGGTGGAGCATGTGGTTTAATTCGATGCAACGCGAAGAACCTTAC | |
| | CTACTCTTGACATCCAGAGAATTTGCTAGAGATAGCTTAGTGCCTTCGGGAACTCTGAGAC | |
| | AGGTGCTGCATGGCTGTCGTCAGCTCGTGTTGTGAAATGTTGGGTTAAGTCCCGTAACGAG | |
| | CGCAACCCCTATCCTTATTTGCCAGCGAGTTAAGTCGGGAACTCTAAGGAGACTGCCGGTG | |
| | ACAAACCGGAGGAAGGTGGGGATGACGTCAAGTCATCATGGCCCTTACGAGTAGGGCTACA | |
| | CACGTGCTACAATGGTTGGTACAGCAGGTTGCTAACCCGCGAGGGGGCGCTAATCCGTCAA | |
| | AACCGATCGTAGTCCGGATCGCAGTCTGCAACTCGACTGCGTGAAGTCGGAATCGCTAGTA | |
| | ATCGCGGATCAGAATGCCGCGGTGAATACGTTCCCGGGCCTTGTACACACCGCCCGTCACA | |
| | CCATGGGAGTTGATTGCACCAGAAGTAGCTAGCTTAACCTTCGGGAGGGCGGTTACCACGG | |
| | TGTGGTTAATGACTGaGGTGAAGTCGTAACAAGGTAGCCGTAGGGGAACCTGCGGCTGGAT | |
| | CATGTGGTTA | |
| P. | GAACTGAAGAGTTTGATCATGGCTCAGATTGAACGCTGGCGGCAGGCCTAACACATGCAAG | 56 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| U409C, | GGGAGAAAGTGGGGGATCTTCGGACCTCACGCTATCAGATGAGCCTAGGTCGGATTAGCTA | |
| G1487A | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGCGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTAGGGTGAAGTCGTAACAAGGTAGCCGTAGGGGAACCTGCGGCTGGA | |
| | TCATGTGGTTA | |
| A. | TAACTGAAGAGTTTGATCATGGCTCAGATTGAACGCTGGCGGCAGGCTTAACACATGCAAG | 57 |
| baumannii | TCGAGCGGGGGAAGGTAGCTTGCTACCGGACCTAGCGGCGGACGGGTGAGTAATGCTTAGG | |
| 16S rRNA, | AATCTGCCTATTAGTGGGGGACAACATCTCGAAAGGGATGCTAATACCGCATACGTCCTAC | |
| U409C, | GGGAGAAAGCAGGGGATCTTCGGACCTTGCGCTAATAGATGAGCCTAAGTCGGATTAGCTA | |
| G1487A | GTTGGTGGGGTAAAGGCCTACCAAGGCGACGATCTGTAGCGGGTCTGAGAGGATGATCCGC | |
| | CACACTGGGACTGAGACACGGCCCAGACTCCTACGGGAGGCAGCAGTGGGGAATATTGGAC | |
| | AATGGGCGCAAGCCTGATCCAGCCATGCCGCGTGTGcGAAGAAGGCCTTATGGTTGTAAAG | |
| | CACTTTAAGCGAGGAGGAGGCTACTTTAGATAATACCTAGAGATAGTGGACGTTACTCGCA | |
| | GAATAAGCACCGGCTAACTCTGTGCCAGCAGCCGCGGTAATACAGAGGGTGCAAGCGTTAA | |
| | TCGGATTTACTGGGCGTAAAGCGCGCGTAGGCGGCTAATTAAGTCAAATGTGAAATCCCCG | |
| | AGCTTAACTTGGGAATTGCATTCGATACTGGTTAGCTAGAGTGTGGGAGAGGATGGTAGAA | |
| | TTCCAGGTGTAGCGGTGAAATGCGTAGAGATCTGGAGGAATACCGATGGCGAAGGCAGCCA | |
| | TCTGGCCTAACACTGACGCTGAGGTGCGAAAGCATGGGGAGCAAACAGGATTAGATACCCT | |
| | GGTAGTCCATGCCGTAAACGATGTCTACTAGCCGTTGGGGCCTTTGAGGCTTTAGTGGCGC | |
| | AGCTAACGCGATAAGTAGACCGCCTGGGGAGTACGGTCGCAAGACTAAAACTCAAATGAAT | |
| | TGACGGGGGCCCGCACAAGCGGTGGAGCATGTGGTTTAATTCGATGCAACGCGAAGAACCT | |
| | TACCTGGCCTTGACATAGTAAGAACTTTCCAGAGATGGATTGGTGCCTTCGGGAACTTACA | |
| | TACAGGTGCTGCATGGCTGTCGTCAGCTCGTGTCGTGAGATGTTGGGTTAAGTCCCGCAAC | |
| | GAGCGCAACCCTTTTCCTTATTTGCCAGCGAGTAATGTCGGGAACTTTAAGGATACTGCCA | |
| | GTGACAAACTGGAGGAAGGCGGGGACGACGTCAAGTCATCATGGCCCTTACGGCCAGGGCT | |
| | ACACACGTGCTACAATGGTCGGTACAAAGGGTTGCTACACAGCGATGTGATGCTAATCTCA | |
| | AAAAGCCGATCGTAGTCCGGATTGGAGTCTGCAACTCGACTCCATGAAGTCGGAATCGCTA | |
| | GTAATCGCGGATCAGAATGCCGCGGTGAATACGTTCCCGGGCCTTGTACACACCGCCCGTC | |
| | ACACCATGGGAGTTTGTTGCACCAGAAGTAGCTAGCCTAACTGCAAAGAGGGCGGTTACCA | |
| | CGGTGTGGCCGATGACTGaGGTGAAGTCGTAACAAGGTAGCCGTAGGGGAACCTGCGGCTG | |
| | GATCATGTGGTAT | |
| A. | AAACTGAAGAGTTTGATCCTGGCTCAGATTGAACGCTAGCGGGATGCTTTACACATGCAAG | 58 |
| faecalis | TCGAACGGCAGCGCGAGAGAGCTTGCTCTCTTGGCGGCGAGTGGCGGACGGGTGAGTAATA | |
| 16S rRNA, | TATCGGAACGTGCCCAGTAGCGGGGGATAACTACTCGAAAGAGTGGCTAATACCGCATACG | |
| U409C, | CCCTACGGGGGAAAGGGGGGGATCGCAAGACCTCTCACTATTGGAGCGGCCGATATCGGAT | |
| G1487A | TAGCTAGTTGGTGGGGTAAAGGCTCACCAAGGCAACGATCCGTAGCTGGTTTGAGAGGACG | |
| | ACCAGCCACACTGGGACTGAGACACGGCCCAGACTCCTACGGGAGGCAGCAGTGGGGAATT | |
| | TTGGACAATGGGGGAAACCCTGATCCAGCCATCCCGCGTGTAcGATGAAGGCCTTCGGGTT | |
| | GTAAAGTACTTTTGGCAGAGAAGAAAAGGTAcCtCCTAATACGaGgTACTGCTGACGGTAT | |
| | CTGCAGAATAAGCACCGGCTAACTACGTGCCAGCAGCCGCGGTAATACGTAGGGTGCAAGC | |
| | GTTAATCGGAATTACTGGGCGTAAAGCGTGTGTAGGCGGTTCGGAAAGAAAGATGTGAAAT | |
| | CCCAGGGCTCAACCTTGGAACTGCATTTTTAACTGCCGAGCTAGAGTATGTCAGAGGGGGG | |
| | TAGAATTCCACGTGTAGCAGTGAAATGCGTAGATATGTGGAGGAATACCGATGGCGAAGGC | |
| | AGCCCCCTGGGATAATACTGACGCTCAGACACGAAAGCGTGGGGAGCAAACAGGATTAGAT | |
| | ACCCTGGTAGTCCACGCCCTAAACGATGTCAACTAGCTGTTGGGGCCGTTAGGCCTTAGTA | |
| | GCGCAGCTAACGCGTGAAGTTGACCGCCTGGGGAGTACGGTCGCAAGATTAAAACTCAAAG | |
| | GAATTGACGGGGACCCGCACAAGCGGTGGATGATGTGGATTAATTCGATGCAACGCGAAAA | |
| | ACCTTACCTACCCTTGACATGTCTGGAAAGCCGAAGAGATTTGGCCGTGCTCGCAAGAGAA | |
| | CCGGAACACAGGTGCTGCATGGCTGTCGTCAGCTCGTGTCGTGAGATGTTGGGTTAAGTCC | |
| | CGCAACGAGCGCAACCCTTGTCATTAGTTGCTACGCAAGAGCACTCTAATGAGACTGCCGG | |
| | TGACAAACCGGAGGAAGGTGGGGATGACGTCAAGTCCTCATGGCCCTTATGGGTAGGGCTT | |
| | CACACGTCATACAATGGTCGGGACAGAGGGTCGCCAACCCGCGAGGGGGAGCCAATCTCAG | |
| | AAACCCGATCGTAGTCCGGATCGCAGTCTGCAACTCGACTGCGTGAAGTCGGAATCGCTAG | |
| | TAATCGCGGATCAGAATGTCGCGGTGAATACGTTCCCGGGTCTTGTACACACCGCCCGTCA | |
| | CACCATGGGAGTGGGTTTCACCAGAAGTAGGTAGCCTAACCGTAAGGAGGGCGCTTACCAC | |
| | GGTGGGATTCATGACTGaGGTGAAGTCGTAACAAGGTAGCCGTATCGGAAGGTGCGGCTGG | |
| | ATCATGTGGTTT | |
| B. | GAACTGAAGAGTTTGATCCTGGCTCAGATTGAACGCTGGCGGGATGCTTTACACATGCAAG | 59 |
| pertussis | TCGGACGGCAGCACGGGCTTCGGCCTGGTGGCGAGTGGCGAACGGGTGAGTAATGTATCGG | |
| 16S rRNA, | AACGTGCCCAGTAGCGGGGGATAACTACGCGAAAGCGTGGCTAATACCGCATACGCCCTAC | |
| U409C, | GGGGGAAAGCGGGGGACCTTCGGGCCTCGCACTATTGGAGCGGCCGATATCGGATTAGCTA | |
| G1487A | GTTGGTGGGGTAACGGCCTACCAAGGCGACGATCCGTAGCTGGTTTGAGAGGACGACCAGC | |
| | CACACTGGGACTGAGACACGGCCCAGACTCCTACGGGAGGCAGCAGTGGGGAATTTTGGAC | |
| | AATGGGGGCAACCCTGATCCAGCCATCCCGCGTGTGCGATGAAGGCCTTCGGGTTGTAAAG | |
| | CACTTTTGGCAGGAAAGAAACGGCACGGGCTAATATCCTGTGCAACTGACGGTACCTGCAG | |
| | AATAAGCACCGGCTAACTACGTGCCAGCAGCCGCGGTAATACGTAGGGTGCAAGCGTTAAT | |
| | CGGAATTACTGGGCGTAAAGCGTGCGCAGGCGGTTCGGAAAGAAAGATGTGAAATCCCAGG | |
| | GCTTAACCTTGGAACTGCATTTTTAACTACCGGGCTAGAGTGTGTCAGAGGGAGGTGGAAT | |
| | TCCGCGTGTAGCAGTGAAATGCGTAGATATGCGGAGGAACACCGATGGCGAAGGCAGCCTC | |
| | CTGGGATAACACTGACGCTCATGCACGAAAGTGTGGGGAGCAAACAGGATTAGATACCCTG | |
| | GTAGTCCACGCCCTAAACGATGTCAACTAGCTGTTGGGGCCTTCGGGCCTTGGTAGCGCAG | |
| | CTAACGCGTGAAGTTGACCGCCTGGGGAGTACGGTCGCAAGATTAAAACTCAAAGGAATTG | |
| | ACGGGGACCCGCACAAGCGGTGGATGATGTGGATTAATTCGATGCAACGCGAAAAACCTTA | |
| | CCTACCCTTGACATGTCTGGAATCCCGAAGAGATTTGGGAGTGCTCGCAAGAGAACCGGAA | |
| | CACAGGTGCTGCATGGCTGTCGTCAGCTCGTGTCGTGAGATGTTGGGTTAAGTCCCGCAAC | |
| | GAGCGCAACCCTTGTCATTAGTTGCTACGAAAGGGCACTCTAATGAGACTGCCGGTGACAA | |
| | ACCGGAGGAAGGTGGGGATGACGTCAAGTCCTCATGGCCCTTATGGGTAGGGCTTCACACG | |
| | TCATACAATGGTCGGGACAGAGGGTTGCCAACCCGCGAGGGGGAGCCAATCCCAGAAACCC | |
| | GGTCGTAGTCCGGATCGCAGTCTGCAACTCGACTGCGTGAAGTCGGAATCGCTAGTAATCG | |
| | CGGATCAGCATGTCGCGGTGAATACGTTCCCGGGTCTTGTACACACCGCCCGTCACACCAT | |
| | GGGAGTGGGTTTTACCAGAAGTAGTTAGCCTAACCGCAAGGGGGGCGATTACCACGGTAGG | |
| | ATTCATGACTGaGGTGAAGTCGTAACAAGGTAGCCGTATCGGAAGGTGCGGCTGGATCATG | |
| | TGGTTT | |
| B. | GAACTGAAGAGTTTGATCCTGGCTCAGATTGAACGCTGGCGGCATGCCTTACACATGCAAG | 60 |
| cenocepacia | TCGAACGGCAGCACGGGTGCTTGCACCTGGTGGCGAGTGGCGAACGGGTGAGTAATACATC | |
| 16S rRNA, | GGAACATGTCCTGTAGTGGGGGATAGCCCGGCGAAAGCCGGATTAATACCGCATACGATCC | |
| U409C, | ATGGATGAAAGCGGGGGACCTTCGGGCCTCGCGCTATAGGGTTGGCCGATGGCTGATTAGC | |
| G1487A | TAGTTGGTGGGGTAAAGGCCTACCAAGGCGACGATCAGTAGCTGGTCTGAGAGGACGACCA | |
| | GCCACACTGGGACTGAGACACGGCCCAGACTCCTACGGGAGGCAGCAGTGGGGAATTTTGG | |
| | ACAATGGGCGAAAGCCTGATCCAGCAATGCCGCGTGTGcGAAGAAGGCCTTCGGGTTGTAA | |
| | AGCACTTTTGTCCGGAAAGAAATCCTTGGCTCTAATACAGTCGGGGGATGACGGTACCGGA | |
| | AGAATAAGCACCGGCTAACTACGTGCCAGCAGCCGCGGTAATACGTAGGGTGCGAGCGTTA | |
| | ATCGGAATTACTGGGCGTAAAGCGTGCGCAGGCGGTTTGCTAAGACCGATGTGAAATCCCC | |
| | GGGCTCAACCTGGGAACTGCATTGGTGACTGGCAGGCTAGAGTATGGCAGAGGGGGGTAGA | |
| | ATTCCACGTGTAGCAGTGAAATGCGTAGAGATGTGGAGGAATACCGATGGCGAAGGCAGCC | |
| | CCCTGGGCCAATACTGACGCTCATGCACGAAAGCGTGGGGAGCAAACAGGATTAGATACCC | |
| | TGGTAGTCCACGCCCTAAACGATGTCAACTAGTTGTTGGGGATTCATTTCCTTAGTAACGT | |
| | AGCTAACGCGTGAAGTTGACCGCCTGGGGAGTACGGTCGCAAGATTAAAACTCAAAGGAAT | |
| | TGACGGGGACCCGCACAAGCGGTGGATGATGTGGATTAATTCGATGCAACGCGAAAAACCT | |
| | TACCTACCCTTGACATGGTCGGAATCCTGCTGAGAGGTGGGAGTGCTCGAAAGAGAACCGG | |
| | CGCACAGGTGCTGCATGGCTGTCGTCAGCTCGTGTCGTGAGATGTTGGGTTAAGTCCCGCA | |
| | ACGAGCGCAACCCTTGTCCTTAGTTGCTACGCAAGAGCACTCTAAGGAGACTGCCGGTGAC | |
| | AAACCGGAGGAAGGTGGGGATGACGTCAAGTCCTCATGGCCCTTATGGGTAGGGCTTCACA | |
| | CGTCATACAATGGTCGGAACAGAGGGTTGCCAACCCGCGAGGGGGAGCTAATCCCAGAAAA | |
| | CCGATCGTAGTCCGGATTGCACTCTGCAACTCGAGTGCATGAAGCTGGAATCGCTAGTAAT | |
| | CGCGGATCAGCATGCCGCGGTGAATACGTTCCCGGGTCTTGTACACACCGCCCGTCACACC | |
| | ATGGGAGTGGGTTTTACCAGAAGTGGCTAGTCTAACCGCAAGGAGGACGGTCACCACGGTA | |
| | GGATTCATGACTGaGGTGAAGTCGTAACAAGGTAGCCGTATCGGAAGGTGCGGCTGGATCA | |
| | TGTGGTTT | |
| N. | GAACATAAGAGTTTGATCCTGGCTCAGATTGAACGCTGGCGGCATGCTTTACACATGCAAG | 61 |
| gonorrhoeae | TCGGACGGCAGCACAGGGAAGCTTGCTTCTCGGGTGGCGAGTGGCGAACGGGTGAGTAACA | |
| 16S rRNA, | TATCGGAACGTACCGGGTAGCGGGGGATAACTGATCGAAAGATCAGCTAATACCGCATACG | |
| U409C, | TCTTGAGAGGGAAAGCAGGGGACCTTCGGGCCTTGCGCTATCCGAGCGGCCGATATCTGAT | |
| G1487A | TAGCTGGTTGGCGGGGTAAAGGCCCACCAAGGCGACGATCAGTAGCGGGTCTGAGAGGATG | |
| | ATCCGCCACACTGGGACTGAGACACGGCCCAGACTCCTACGGGAGGCAGCAGTGGGGAATT | |
| | TTGGACAATGGGCGCAAGCCTGATCCAGCCATGCCGCGTGTCCGAAGAAGGCCTTCGGGTT | |
| | GTAAAGGACTTTTGTCAGGGAAGAAAAGGCCGTTGCCAATATCGGCGGCCGATGACGGTAC | |
| | CTGAAGAATAAGCACCGGCTAACTACGTGCCAGCAGCCGCGGTAATACGTAGGGTGCGAGC | |
| | GTTAATCGGAATTACTGGGCGTAAAGCGGGCGCAGACGGTTACTTAAGCAGGATGTGAAAT | |
| | CCCCGGGCTCAACCCGGGAACTGCGTTCTGAACTGGGTGACTCGAGTGTGTCAGAGGGAGG | |
| | TGGAATTCCACGTGTAGCAGTGAAATGCGTAGAGATGTGGAGGAATACCGATGGCGAAGGC | |
| | AGCCTCCTGGGATAACACTGACGTTCATGTCCGAAAGCGTGGGTAGCAAACAGGATTAGAT | |
| | ACCCTGGTAGTCCACGCCCTAAACGATGTCAATTAGCTGTTGGGCAACTTGATTGCTTGGT | |
| | AGCGTAGCTAACGCGTGAAATTGACCGCCTGGGGAGTACGGTCGCAAGATTAAAACTCAAA | |
| | GGAATTGACGGGGACCCGCACAAGCGGTGGATGATGTGGATTAATTCGATGCAACGCGAAG | |
| | AACCTTACCTGGTTTTGACATGTGCGGAATCCTCCGGAGACGGAGGAGTGCCTTCGGGAGC | |
| | CGTAACACAGGTGCTGCATGGCTGTCGTCAGCTCGTGTCGTGAGATGTTGGGTTAAGTCCC | |
| | GCAACGAGCGCAACCCTTGTCATTAGTTGCCATCATTCGGTTGGGCACTCTAATGAGACTG | |
| | CCGGTGACAAGCCGGAGGAAGGTGGGGATGACGTCAAGTCCTCATGGCCCTTATGACCAGG | |
| | GCTTCACACGTCATACAATGGTCGGTACAGAGGGTAGCCAAGCCGCGAGGCGGAGCCAATC | |
| | TCACAAAACCGATCGTAGTCCGGATTGCACTCTGCAACTCGAGTGCATGAAGTCGGAATCG | |
| | CTAGTAATCGCAGGTCAGCATACTGCGGTGAATACGTTCCCGGGTCTTGTACACACCGCCC | |
| | GTCACACCATGGGAGTGGGGGATACCAGAAGTAGGTAGGGTAACCGCAAGGAGTCCGCTTA | |
| | CCACGGTATGCTTCATGACTCGGGTGAAGTCGTAACAAGGTAGCCGTAGGGGAACCTGCGG | |
| | CTGGATCATGTGGTTT | |
| M. | GGAGAGTTTGATCCTGGCTCAGAACGAACGCTGGCGGCGTGCCTAACACATGCAAGTCGAA | 62 |
| ferrooxydans | CGGACTTTAAGACTTCGGTTTTAAAGTTAGTGGCGCACGGGTGAGTAACGCGTGGATATCT | |
| 16S rRNA, | GCCTATCAGTGGGGGACAACTACAGGAAACTGTAGCTAATACCGCATACGCTGTACGCAGG | |
| U409C, | AAAGCGGGGGATCTTCGGACCTCGCGCTGATAGATGAGTCCGCGTCTGATTAGCTAGTTGG | |
| G1487A | TGGGGTAAAGGCCTACCAAGGCGATGATCAGTAACTGGTTTGAGAGGATGATCAGTCACAC | |
| | TGGAACTGAGACACGGTCCAGACTCCTACGGGAGGCAGCAGTGGGGAATATTGCACAATGG | |
| | GGGAAACCCTGATGCAGCGACGCCGCGTGCGCGAAGAAGGCCTTCGGGTTGTAAACCGCTT | |
| | TCAATTGGGAAGAAGAGATGAGTACTAATACTGCTCTGATTTGACGGTACCTTTAGAAGAA | |
| | GCACCGGCTAATTTCGTGCCAGCAGCCGCGGTAATACGAAAGGTGCAAGCGTTGTTCGGAT | |
| | TTACTGGGCGTAAAGAGATCGTAGGTGGTTTGTTAAGTCGGATGTGAAATCCCAGGGCTCA | |
| | ACCCTGGAACTGCATTCGATACTGGCAGACTAGAGTTTGGGAGGGGTAAGCGGAATTCCGT | |
| | GTGTAGCAGTGAAATGCGTAGATATACGGAGGAACACCTGAGGCGAAGGCGGCTTACTGGA | |
| | CCAATACTGACACTGAGGATCGAAAGCGTGGGTAGCAAACAGGATTAGATACCCTGGTAGT | |
| | CCACGCCCTAAACGATGTCAACTAGCCGTAGCGGGTATCGACCCCTGCTGTGGCGAAGCTA | |
| | ACGCGATAAGTTGACCGCCTGGGGAGTACGGTCGCAAGATTAAAACTCAAAGGAATTGACG | |
| | GGGGCCCGCACAAGCGGTGGAGCATGTGGTTTAATTCGACGCAACGCGAAGAACCTTACCT | |
| | GGGTTTGACATCCTAAGAATACTTTAGAGATAGAGTAGTGCCTTCGGGAGCTTAGAGACAG | |
| | GTGCTGCATGGCTGTCGTCAGCTCGTGTCGTGAGATGTTGGGTTAAGTCCCGCAACGAGCG | |
| | CAACCCCTATCGTTAGTTGCCATCATTTAGTTGGGCACTCTAGCGAGACTGCCGGTGACAA | |
| | ACCGGAGGAAGGTGGGGACGACGTCAAGTCATCATGGCCCTTATGTCCAGGGCTACACACG | |
| | TGCTACAATGGCGACTACAACAAGTTGCGAACCCGCGAGGGGGAGCCAATCTTATAAAAGT | |
| | CGTCTCAGTTCGGATTGGAGTCTGCAACTCGACTCCATGAAGTTGGAATCGCTAGTAATCG | |
| | CGGATCAGCATGCCGCGGTGAATACGTTCCCGGGCCTTGTACACACCGCCCGTCACACCAT | |
| | GGGAATTGGTTGCACCAGAAGCCGCCGGGCTAACCTTCGGGAGGCAGGCGTCTACGGTGTG | |
| | GTCGGTAACTGaGGTGAAGTCGTAACAAGGTATCCCTACCGGAAGGTGGGGATGGATCATG | |
| | TGGTTT | |
| C. | CAACCTGAGAGTTTGATCCTGGCTCAGAGCGAACGCTGGCGGCAGGCCTAACACATGCAAG | 63 |
| crescentus | TCGAACGGATCCTTCGGGATTAGTGGCGGACGGGTGAGTAACACGTGGGAACGTGCCCTTT | |
| 16S rRNA, | GGTTCGGAACAACTCAGGGAAACTTGAGCTAATACCGGATGTGCCCTTCGGGGGAAAGATT | |
| U409C, | TATCGCCATTGGAGCGGCCCGCGTCTGATTAGCTAGTTGGTGAGGTAAAGGCTCACCAAGG | |
| G1487A | CGACGATCAGTAGCTGGTCTGAGAGGATGATCAGCCACATTGGGACTGAGACACGGCCCAA | |
| | ACTCCTACGGGAGGCAGCAGTGGGGAATCTTGCGCAATGGGCGAAAGCCTGACGCAGCCAT | |
| | GCCGCGTGAAcGATGAAGGTCTTAGGATTGTAAAATTCTTTCACCGGGGACGATAATGACG | |
| | GTACCCGGAGAAGAAGCCCCGGCTAACTTCGTGCCAGCAGCCGCGGTAATACGAAGGGGGC | |
| | TAGCGTTGCTCGGAATTACTGGGCGTAAAGGGAGCGTAGGCGGACTGTTTAGTCAGAGGTG | |
| | AAAGCCCAGGGCTCAACCTTGGAATTGCCTTTGATACTGGCAGTCTTGAGTACGGAAGAGG | |
| | TATGTGGAACTCCGAGTGTAGAGGTGAAATTCGTAGATATTCGGAAGAACACCAGTGGCGA | |
| | AGGCGACATACTGGTCCGTTACTGACGCTGAGGCTCGAAAGCGTGGGGAGCAAACAGGATT | |
| | AGATACCCTGGTAGTCCACGCCGTAAACGATGAGTGCTAGTTGTCGGCATGCATGCATGTC | |
| | GGTGACGCAGCTAACGCATTAAGCACTCCGCCTGGGGAGTACGGTCGCAAGATTAAAACTC | |
| | AAAGGAATTGACGGGGGCCCGCACAAGCGGTGGAGCATGTGGTTTAATTCGAAGCAACGCG | |
| | CAGAACCTTACCACCTTTTGACATGCCTGGACCGCCACAGAGATGTGGTTTTCCCTTCGGG | |
| | GACTGGGACACAGGTGCTGCATGGCTGTCGTCAGCTCGTGTCGTGAGATGTTGGGTTAAGT | |
| | CCCGCAACGAGCGCAACCCTCGTGATTAGTTGCCATCAGGTTTGGCTGGGCACTCTAATCA | |
| | TACTGCCGGAGTTAATCCGGAGGAAGGCGGGGATGACGTCAAGTCCTCATGGCCCTTACAA | |
| | GGTGGGCTACACACGTGCTACAATGGCGACTACAGAGGGCTGCAATCCCGCGAGGGGGAGC | |
| | CAATCCCTAAAAGTCGTCTCAGTTCGGATTGTTCTCTGCAACTCGAGAGCATGAAGTTGGA | |
| | ATCGCTAGTAATCGCGGATCAGCATGCCGCGGTGAATACGTTCCCGGGCCTTGTACACACC | |
| | GCCCGTCACACCATGGGAGTTGGCTTTACCCGAAGGCGCTGCGCTAACCGCAAGGGGGCAG | |
| | GCGACCACGGTAGGGTCAGCGACTGaGGTGAAGTCGTAACAAGGTAGCCGTAGGGGAACCT | |
| | GCGGCTGGATCATGTGGTTT | |
| E. coli | AAATTGAAGAGTTTGATCATGGCTCAGATTGAACGCTGGCGGCAGGCCTAACACATGCAA | 64 |
| 16S rRNA, | GTCGAACGGTAACAGGAAGAAGCTTGCTTCTTTGCTGACGAGTGGCGGACGGGTGAGTAAT | |
| C440U | GTCTGGGAAACTGCCTGATGGAGGGGGATAACTACTGGAAACGGTAGCTAATACCGCATAA | |
| | CGTCGCAAGACCAAAGAGGGGGACCTTCGGGCCTCTTGCCATCGGATGTGCCCAGATGGGA | |
| | TTAGCTAGTAGGTGGGGTAACGGCTCACCTAGGCGACGATCCCTAGCTGGTCTGAGAGGAT | |
| | GACCAGCCACACTGGAACTGAGACACGGTCCAGACTCCTACGGGAGGCAGCAGTGGGGAAT | |
| | ATTGCACAATGGGCGCAAGCCTGATGCAGCCATGCCGCGTGTATGAAGAAGGCCTTCGGGT | |
| | TGTAAAGTACTTTtAGCGGGGAGGAAGGGAGTAAAGTTAATACCTTTGCTCATTGACGTTA | |
| | CCCGCAGAAGAAGCACCGGCTAACTCCGTGCCAGCAGCCGCGGTAATACGGAGGGTGCAAG | |
| | CGTTAATCGGAATTACTGGGCGTAAAGCGCACGCAGGCGGTTTGTTAAGTCAGATGTGAAA | |
| | TCCCCGGGCTCAACCTGGGAACTGCATCTGATACTGGCAAGCTTGAGTCTCGTAGAGGGGG | |
| | GTAGAATTCCAGGTGTAGCGGTGAAATGCGTAGAGATCTGGAGGAATACCGGTGGCGAAGG | |
| | CGGCCCCCTGGACGAAGACTGACGCTCAGGTGCGAAAGCGTGGGGAGCAAACAGGATTAGA | |
| | TACCCTGGTAGTCCACGCCGTAAACGATGTCGACTTGGAGGTTGTGCCCTTGAGGCGTGGC | |
| | TTCCGGAGCTAACGCGTTAAGTCGACCGCCTGGGGAGTACGGCCGCAAGGTTAAAACTCAA | |
| | ATGAATTGACGGGGGCCCGCACAAGCGGTGGAGCATGTGGTTTAATTCGATGCAACGCGAA | |
| | GAACCTTACCTGGTCTTGACATCCACGGAAGTTTTCAGAGATGAGAATGTGCCTTCGGGAA | |
| | CCGTGAGACAGGTGCTGCATGGCTGTCGTCAGCTCGTGTTGTGAAATGTTGGGTTAAGTCC | |
| | CGCAACGAGCGCAACCCTTATCCTTTGTTGCCAGCGGTCCGGCCGGGAACTCAAAGGAGAC | |
| | TGCCAGTGATAAACTGGAGGAAGGTGGGGATGACGTCAAGTCATCATGGCCCTTACGACCA | |
| | GGGCTACACACGTGCTACAATGGCGCATACAAAGAGAAGCGACCTCGCGAGAGCAAGCGGA | |
| | CCTCATAAAGTGCGTCGTAGTCCGGATTGGAGTCTGCAACTCGACTCCATGAAGTCGGAAT | |
| | CGCTAGTAATCGTGGATCAGAATGCCACGGTGAATACGTTCCCGGGCCTTGTACACACCGC | |
| | CCGTCACACCATGGGAGTGGGTTGCAAAAGAAGTAGGTAGCTTAACCTTCGGGAGGGCGCT | |
| | TACCACTTTGTGATTCATGACTGGGGTGAAGTCGTAACAAGGTAACCGTAGGGGAACCTGC | |
| | GGTTGGATCAtgtggtTA | |
| E. coli | AAATTGAAGAGTTTGATCATGGCTCAGATTGAACGCTGGCGGCAGGCCTAACACATGCAAG | 65 |
| 16S rRNA, | TCGAACGGTAACAGGAAGAAGCTTGCTTCTTTGCTGACGAGTGGCGGACGGGTGAGTAATG | |
| U904C | TCTGGGAAACTGCCTGATGGAGGGGGATAACTACTGGAAACGGTAGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCAtgtggtTA | |
| E. coli | AAATTGAAGAGTTTGATCATGGCTCAGATTGAACGCTGGCGGCAGGCCTAACACATGCAAG | 66 |
| 16S rRNA, | TCGAACGGTAACAGGAAGAAGCTTGCTTCTTTGCTGACGAGTGGCGGACGGGTGAGTAATG | |
| A906G | TCTGGGAAACTGCCTGATGGAGGGGGATAACTACTGGAAACGGTAGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTgAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCAtgtggtTA | |
| E. coli | AAATTGAAGAGTTTGATCATGGCTCAGATTGAACGCTGGCGGCAGGCCTAACACATGCAAG | 67 |
| 16S rRNA, | TCGAACGGTAACAGGAAGAAGCTTGCTTCTTTGCTGACGAGTGGCGGACGGGTGAGTAATG | |
| C1098U | TCTGGGAAACTGCCTGATGGAGGGGGATAACTACTGGAAACGGTAGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCt | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCAtgtggtTA | |
| E. coli | AAATTGAAGAGTTTGATCATGGCTCAGATTGAACGCTGGCGGCAGGCCTAACACATGCAAG | 68 |
| 16S rRNA, | TCGAACGGTAACAGGAAGAAGCTTGCTTCTTTGCTGACGAGTGGCGGACGGGTGAGTAATG | |
| G1415A | TCTGGGAAACTGCCTGATGGAGGGGGATAACTACTGGAAACGGTAGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATaGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCAtgtggtTA | |
| P. | GAACTGAAGAGTTTGATCATGGCTCAGATTGAACGCTGGCGGCAGGCCTAACACATGCAAG | 69 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| U409C | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGcGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 70 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| C440U | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTtAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 71 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| U904C | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGcTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 72 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| A906G | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTgAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 73 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| C1098U | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCEGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 74 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| G1415A | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATaGGAGTGGGTTGCTCCAGAAGTAGCTAGTCTAACCGCAAGGGGGACGGTTACCACG | |
| | GAGTGATTCATGACTGGGGTGAAGTCGTAACAAGGTAGCCGTAGGGGAACCTGCGGCTGGA | |
| | TCATGTGGTTA | |
| P. | GAACTGAAGAGTTTGATCATGGCTCAGATTGAACGCTGGCGGCAGGCCTAACACATGCAAG | 75 |
| aeruginosa | TCGAGCGGATGAAGGGAGCTTGCTCCTGGATTCAGCGGCGGACGGGTGAGTAATGCCTAGG | |
| 16S rRNA, | AATCTGCCTGGTAGTGGGGGATAACGTCCGGAAACGGGCGCTAATACCGCATACGTCCTGA | |
| G1487A | GGGAGAAAGTGGGGGATCTTCGGACCTCACGCTATCAGATGAGCCTAGGTCGGATTAGCTA | |
| | GTTGGTGGGGTAAAGGCCTACCAAGGCGACGATCCGTAACTGGTCTGAGAGGATGATCAGT | |
| | CACACTGGAACTGAGACACGGTCCAGACTCCTACGGGAGGCAGCAGTGGGGAATATTGGAC | |
| | AATGGGCGAAAGCCTGATCCAGCCATGCCGCGTGTGTGAAGAAGGTCTTCGGATTGTAAAG | |
| | CACTTTAAGTTGGGAGGAAGGGCAGTAAGTTAATACCTTGCTGTTTTGACGTTACCAACAG | |
| | AATAAGCACCGGCTAACTTCGTGCCAGCAGCCGCGGTAATACGAAGGGTGCAAGCGTTAAT | |
| | CGGAATTACTGGGCGTAAAGCGCGCGTAGGTGGTTCAGCAAGTTGGATGTGAAATCCCCGG | |
| | GCTCAACCTGGGAACTGCATCCAAAACTACTGAGCTAGAGTACGGTAGAGGGTGGTGGAAT | |
| | TTCCTGTGTAGCGGTGAAATGCGTAGATATAGGAAGGAACACCAGTGGCGAAGGCGACCAC | |
| | CTGGACTGATACTGACACTGAGGTGCGAAAGCGTGGGGAGCAAACAGGATTAGATACCCTG | |
| | GTAGTCCACGCCGTAAACGATGTCGACTAGCCGTTGGGATCCTTGAGATCTTAGTGGCGCA | |
| | GCTAACGCGATAAGTCGACCGCCTGGGGAGTACGGCCGCAAGGTTAAAACTCAAATGAATT | |
| | GACGGGGGCCCGCACAAGCGGTGGAGCATGTGGTTTAATTCGAAGCAACGCGAAGAACCTT | |
| | ACCTGGCCTTGACATGCTGAGAACTTTCCAGAGATGGATTGGTGCCTTCGGGAACTCAGAC | |
| | ACAGGTGCTGCATGGCTGTCGTCAGCTCGTGTCGTGAGATGTTGGGTTAAGTCCCGTAACG | |
| | AGCGCAACCCTTGTCCTTAGTTACCAGCACCTCGGGTGGGCACTCTAAGGAGACTGCCGGT | |
| | GACAAACCGGAGGAAGGTGGGGATGACGTCAAGTCATCATGGCCCTTACGGCCAGGGCTAC | |
| | ACACGTGCTACAATGGTCGGTACAAAGGGTTGCCAAGCCGCGAGGTGGAGCTAATCCCATA | |
| | AAACCGATCGTAGTCCGGATCGCAGTCTGCAACTCGACTGCGTGAAGTCGGAATCGCTAGT | |
| | AATCGTGAATCAGAATGTCACGGTGAATACGTTCCCGGGCCTTGTACACACCGCCCGTCAC | |
| | ACCATGGGAGTGGGTTGCTCCAGAAGTAGCTAGTCTAACCGCAAGGGGGACGGTTACCACG | |
| | GAGTGATTCATGACTGaGGTGAAGTCGTAACAAGGTAGCCGTAGGGGAACCTGCGGCTGGA | |
| | TCATGTGGTTA | |
| V. | TAATTGAAGAGTTTGATCATGGCTCAGATTGAACGCTGGCGGCAGGCCTAACACATGCAAG | 76 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| U409C | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTAcGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 77 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| C440U | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTtAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 78 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| U904C | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATCAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 79 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| A906G | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAgAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 80 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| C1098U | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCt | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 81 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| G1415A | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATaGGAGTGGGCTGCAAAAGAAGCAGGTAGTTTAACCTTCGGGAGGACGCT | |
| | TGCCACTTTGTGGTTCATGACTGGGGTGAAGTCGTAACAAGGTAGCGCTAGGGGAACCTGG | |
| | CGCTGGATCATGTGGTTA | |
| V. | TAATTGAAGAGTTTGATCATGGCTCAGATTGAACGCTGGCGGCAGGCCTAACACATGCAAG | 82 |
| cholerae | TCGAGCGGCAGCACAGAGGAACTTGTTCCTTGGGTGGCGAGCGGCGGACGGGTGAGTAATG | |
| 16S rRNA, | CCTGGGAAATTGCCCGGTAGAGGGGGATAACCATTGGAAACGATGGCTAATACCGCATAAC | |
| G1487A | CTCGCAAGAGCAAAGCAGGGGACCTTCGGGCCTTGCGCTACCGGATATGCCCAGGTGGGAT | |
| | TAGCTAGTTGGTGAGGTAAGGGCTCACCAAGGCGACGATCCCTAGCTGGTCTGAGAGGATG | |
| | ATCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGTAGGGAGGAAGGTGGTTAAGTTAATACCTTAATCATTTGACGTTAC | |
| | CTACAGAAGAAGCACCGGCTAACTCCGTGCCAGCAGCCGCGGTAATACGGAGGGTGCAAGC | |
| | GTTAATCGGAATTACTGGGCGTAAAGCGCATGCAGGTGGTTTGTTAAGTCAGATGTGAAAG | |
| | CCCTGGGCTCAACCTAGGAATCGCATTTGAAACTGACAAGCTAGAGTACTGTAGAGGGGGG | |
| | TAGAATTTCAGGTGTAGCGGTGAAATGCGTAGAGATCTGAAGGAATACCGGTGGCGAAGGC | |
| | GGCCCCCTGGACAGATACTGACACTCAGATGCGAAAGCGTGGGGAGCAAACAGGATTAGAT | |
| | ACCCTGGTAGTCCACGCCGTAAACGATGTCTACTTGGAGGTTGTGACCTAGAGTCGTGGCT | |
| | TTCGGAGCTAACGCGTTAAGTAGACCGCCTGGGGAGTACGGTCGCAAGATTAAAACTCAAA | |
| | TGAATTGACGGGGGCCCGCACAAGCGGTGGAGCATGTGGTTTAATTCGATGCAACGCGAAG | |
| | AACCTTACCTACTCTTGACATCCAGAGAATCTAGCGGAGACGCTGGAGTGCCTTCGGGAGC | |
| | TCTGAGACAGGTGCTGCATGGCTGTCGTCAGCTCGTGTTGTGAAATGTTGGGTTAAGTCCC | |
| | GCAACGAGCGCAACCCTTATCCTTGTTTGCCAGCACGTAATGGTGGGAACTCCAGGGAGAC | |
| | TGCCGGTGATAAACCGGAGGAAGGTGGGGACGACGTCAAGTCATCATGGCCCTTACGAGTA | |
| | GGGCTACACACGTGCTACAATGGCGTATACAGAGGGCAGCGATACCGCGAGGTGGAGCGAA | |
| | TCTCACAAAGTACGTCGTAGTCCGGATTGGAGTCTGCAACTCGACTCCATGAAGTCGGAAT | |
| | CGCTAGTAATCGCAAATCAGAATGTTGCGGTGAATACGTTCCCGGGCCTTGTACACACCGC | |
| | CCGTCACACCATGGGAGTGGGCTGCAAAAGAAGCAGGTAGTTTAACCTTCGGGAGGACGCT | |
| | TGCCACTTTGTGGTTCATGACTGaGGTGAAGTCGTAACAAGGTAGCGCTAGGGGAACCTGG | |
| | CGCTGGATCATGTGGTTA | |
| GGS₂ | GGSGGS | 83 |
| linker | | |
| maltose | CLSYETEILTVEYGLLPIGKIVEKRIECTVYSVDNNGNIYTQPVAQWHDRGEQEVFEYCLE | 84 |
| binding | DGSLIRATKDHKFMTVDGQMLPIDEIFERELDLMRVDNLPNGGSGGSKIEEGKLVIWINGD | |
| protein | KGYNGLAEVGKKFEKDTGIKVTVEHPDKLEEKFPQVAATGDGPDIIFWAHDREGGYAQSGL | |
| (MBP) | LAEITPDKAFQDKLYPFTWDAVRYNGKLIAYPIAVEALSLIYNKDLLPNPPKTWEEIPALD | |
| intein | KELKAKGKSALMENLQEPYFTWPLIAADGGYAFKYENGKYDIKDVGVDNAGAKAGLTFLVD | |
| sequence | LIKNKHMNADTDYSIAEAAFNKGETAMTINGPWAWSNIDTSKVNYGVTVLPTFKGQPSKPF | |
| | VGVLSAGINAASPNKELAKEFLENYLLTDEGLEAVNKDKPLGAVALKSYEEELAKDPRIAA | |
| | TMENAQKGEIMPNIPQMSAFWYAVRTAVINAASGRQTVDEALKDAQTRITKGGSGGSIKIA | |
| | TRKYLGKQNVYDIGVERDHNFALKNGFIASNCEN | |
| SP_{EC} | atgattgacatgctagttttacgGttaccgttcatcgattctcttgtttgctccagactct | 85 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccccctgcaaa | |
| | aaataaattcatataaaaaacatacagataaccatctgcggtgataaattatctctggcgg | |
| | tgttgacataaataccactggcggttatactgagcacgggtaccGGCCGCTGAGAAAAAGC | |
| | GAAGCGGCACTGCTCTTTAACAATTTATCAGACAATCTGTGTGGGCACTCGAAGATACGGA | |
| | TTCTTAACGTCGCAAGACGAAAAATGAATACCAAGTCTCAAGAGTGAACACGTAATTCATT | |
| | ACGAAGTTTAATTCTTTGAGCGTCAAACTTTTAAATTGAAGAGTTTGATCATGGCTCAGAT | |
| | TGAACGCTGGCGGCAGGCCTAACACATGCAAGTCGAACGGTAACAGGAAGAAGCTTGCTTC | |
| | TTTGCTGACGAGTGGCGGACGGGTGAGTAATGTCTGGGAAACTGCCTGATGGAGGGGGATA | |
| | ACTACTGGAAACGGTAGCTAATACCGCATAACGTCGCAAGACCAAAGAGGGGGACCTTCGG | |
| | GCCTCTTGCCATCGGATGTGCCCAGATGGGATTAGCTAGTAGGTGGGGTAACGGCTCACCT | |
| | AGGCGACGATCCCTAGCTGGTCTGAGAGGATGACCAGCCACACTGGAACTGAGACACGGTC | |
| | CAGACTCCTACGGGAGGCAGCAGTGGGGAATATTGCACAATGGGCGCAAGCCTGATGCAGC | |
| | CATGCCGCGTGTATGAAGAAGGCCTTCGGGTTGTAAAGTACTTTCAGCGGGGAGGAAGGGA | |
| | GTAAAGTTAATACCTTTGCTCATTGACGTTACCCGCAGAAGAAGCACCGGCTAACTCCGTG | |
| | CCAGCAGCCGCGGTAATACGGAGGGTGCAAGCGTTAATCGGAATTACTGGGCGTAAAGCGC | |
| | ACGCAGGCGGTTTGTTAAGTCAGATGTGAAATCCCCGGGCTCAACCTGGGAACTGCATCTG | |
| | ATACTGGCAAGCTTGAGTCTCGTAGAGGGGGGTAGAATTCCAGGTGTAGCGGTGAAATGCG | |
| | TAGAGATCTGGAGGAATACCGGTGGCGAAGGCGGCCCCCTGGACGAAGACTGACGCTCAGG | |
| | TGCGAAAGCGTGGGGAGCAAACAGGATTAGATACCCTGGTAGTCCACGCCGTAAACGATGT | |
| | CGACTTGGAGGTTGTGCCCTTGAGGCGTGGCTTCCGGAGCTAACGCGTTAAGTCGACCGCC | |
| | TGGGGAGTACGGCCGCAAGGTTAAAACTCAAATGAATTGACGGGGGCCCGCACAAGCGGTG | |
| | GAGCATGTGGTTTAATTCGATGCAACGCGAAGAACCTTACCTGGTCTTGACATCCACGGAA | |
| | GTTTTCAGAGATGAGAATGTGCCTTCGGGAACCGTGAGACAGGTGCTGCATGGCTGTCGTC | |
| | AGCTCGTGTTGTGAAATGTTGGGTTAAGTCCCGCAACGAGCGCAACCCTTATCCTTTGTTG | |
| | CCAGCGGTCCGGCCGGGAACTCAAAGGAGACTGCCAGTGATAAACTGGAGGAAGGTGGGGA | |
| | TGACGTCAAGTCATCATGGCCCTTACGACCAGGGCTACACACGTGCTACAATGGCGCATAC | |
| | AAAGAGAAGCGACCTCGCGAGAGCAAGCGGACCTCATAAAGTGCGTCGTAGTCCGGATTGG | |
| | AGTCTGCAACTCGACTCCATGAAGTCGGAATCGCTAGTAATCGTGGATCAGAATGCCACGG | |
| | TGAATACGTTCCCGGGCCTTGTACACACCGCCCGTCACACCATGGGAGTGGGTTGCAAAAG | |
| | AAGTAGGTAGCTTAACCTTCGGGAGGGCGCTTACCACTTTGTGATTCATGACTGGGGTGAA | |
| | GTCGTAACAAGGTAACCGTAGGGGAACCTGCGGTTGGATCACTTGTATACCTTAAAGAAGC | |
| | GTACTTTGTAGTGCTCACACAGATTGTCTGATAGAAAGTGAAAAGCAAGGCGTTTACGCGT | |
| | TGGGAGTGAGGCTGAAGAGAATAAGGCCGTTCGCTTTCTATTAATGAAAGCTCACCCTACA | |
| | CGAAAATATCACGCAACGCGTGATAAGCAATTTTCGTGTCCCCTTCGTCTAGAGGCCCAGG | |
| | ACACCGCCCTTTCACGGCGGTAACAGGGGTTCGAATCCCCTAGGGGACGCCACTTGCTGGT | |
| | TTGTGAGTGAAAGTCGCCGACCTTAATATCTCAAAACTCATCTTCGGGTGATGTTTGAGAT | |
| | ATTTGCTCTTTAAAAATCTGGATCAAGCTGAAAATTGAAACACTGAACAACGAGAGTTGTT | |
| | CGTGAGTCTCTCAAATTTTCGCAACACGATGATGAATCGAAAGAAACATCTTCGGGTTGTG | |
| | AGGTTAAGCGACTAAGCGTACACGGTGGATGCCCTGGCAGTCAGAGGCGATGAAGGACGTG | |
| | CTAATCTGCGATAAGCGTCGGTAAGGTGATATGAACCGTTATAACCGGCGATTTCCGAATG | |
| | GGGAAACCCAGTGTGTTTCGACACACTATCATTAACTGAATCCATAGGTTAATGAGGCGAA | |
| | CCGGGGGAACTGAAACATCTAAGTACCCCGAGGAAAAGAAATCAACCGAGATTCCCCCAGT | |
| | AGCGGCGAGCGAACGGGGAGCAGCCCAGAGCCTGAATCAGTGTGTGTGTTAGTGGAAGCGT | |
| | CTGGAAAGGCGCGCGATACAGGGTGACAGCCCCGTACACAAAAATGCACATGCTGTGAGCT | |
| | CGATGAGTAGGGCGGGACACGTGGTATCCTGTCTGAATATGGGGGGACCATCCTCCAAGGC | |
| | TAAATACTCCTGACTGACCGATAGTGAACCAGTACCGTGAGGGAAAGGCGAAAAGAACCCC | |
| | GGCGAGGGGAGTGAAAAAGAACCTGAAACCGTGTACGTACAAGCAGTGGGAGCACGCTTAG | |
| | GCGTGTGACTGCGTACCTTTTGTATAATGGGTCAGCGACTTATATTCTGTAGCAAGGTTAA | |
| | CCGAATAGGGGAGCCGAAGGGAAACCGAGTCTTAACTGGGCGTTAAGTTGCAGGGTATAGA | |
| | CCCGAAACCCGGTGATCTAGCCATGGGCAGGTTGAAGGTTGGGTAACACTAACTGGAGGAC | |
| | CGAACCGACTAATGTTGAAAAATTAGCGGATGACTTGTGGCTGGGGGTGAAAGGCCAATCA | |
| | AACCGGGAGATAGCTGGTTCTCCCCGAAAGCTATTTAGGTAGCGCCTCGTGAATTCATCTC | |
| | CGGGGGTAGAGCACTGTTTCGGCAAGGGGGTCATCCCGACTTACCAACCCGATGCAAACTG | |
| | CGAATACCGGAGAATGTTATCACGGGAGACACACGGCGGGTGCTAACGTCCGTCGTGAAGA | |
| | GGGAAACAACCCAGACCGCCAGCTAAGGTCCCAAAGTCATGGTTAAGTGGGAAACGATGTG | |
| | GGAAGGCCCAGACAGCCAGGATGTTGGCTTAGAAGCAGCCATCATTTAAAGAAAGCGTAAT | |
| | AGCTCACTGGTCGAGTCGGCCTGCGCGGAAGATGTAACGGGGCTAAACCATGCACCGAAGC | |
| | TGCGGCAGCGACGCTTATGCGTTGTTGGGTAGGGGAGCGTTCTGTAAGCCTGCGAAGGTGT | |
| | GCTGTGAGGCATGCTGGAGGTATCAGAAGTGCGAATGCTGACATAAGTAACGATAAAGCGG | |
| | GTGAAAAGCCCGCTCGCCGGAAGACCAAGGGTTCCTGTCCAACGTTAATCGGGGCAGGGTG | |
| | AGTCGACCCCTAAGGCGAGGCCGAAAGGCGTAGTCGATGGGAAACAGGTTAATATTCCTGT | |
| | ACTTGGTGTTACTGCGAAGGGGGGACGGAGAAGGCTATGTTGGCCGGGCGACGGTTGTCCC | |
| | GGTTTAAGCGTGTAGGCTGGTTTTCCAGGCAAATCCGGAAAATCAAGGCTGAGGCGTGATG | |
| | ACGAGGCACTACGGTGCTGAAGCAACAAATGCCCTGCTTCCAGGAAAAGCCTCTAAGCATC | |
| | AGGTAACATCAAATCGTACCCCAAACCGACACAGGTGGTCAGGTAGAGAATACCAAGGCGC | |
| | TTGAGAGAACTCGGGTGAAGGAACTAGGCAAAATGGTGCCGTAACTTCGGGAGAAGGCACG | |
| | CTGATATGTAGGTGAGGTCCCTCGCGGATGGAGCTGAAATCAGTCGAAGATACCAGCTGGC | |
| | TGCAACTGTTTATTAAAAACACAGCACTGTGCAAACACGAAAGTGGACGTATACGGTGTGA | |
| | CGCCTGCCCGGTGCCGGAAGGTTAATTGATGGGGTTAGCGCAAGCGAAGCTCTTGATCGAA | |
| | GCCCCGGTAAACGGCGGCCGTAACTATAACGGTCCTAAGGTAGCGAAATTCCTTGTCGGGT | |
| | AAGTTCCGACCTGCACGAATGGCGTAATGATGGCCAGGCTGTCTCCACCCGAGACTCAGTG | |
| | AAATTGAACTCGCTGTGAAGATGCAGTGTACCCGCGGCAAGACGGAAAGACCCCGTGAACC | |
| | TTTACTATAGCTTGACACTGAACATTGAGCCTTGATGTGTAGGATAGGTGGGAGGCTTTGA | |
| | AGTGTGGACGCCAGTCTGCATGGAGCCGACCTTGAAATACCACCCTTTAATGTTTGATGTT | |
| | CTAACGTTGACCCGTAATCCGGGTTGCGGACAGTGTCTGGTGGGTAGTTTGACTGGGGCGG | |
| | TCTCCTCCTAAAGAGTAACGGAGGAGCACGAAGGTTGGCTAATCCTGGTCGGACATCAGGA | |
| | GGTTAGTGCAATGGCATAAGCCAGCTTGACTGCGAGCGTGACGGCGCGAGCAGGTGCGAAA | |
| | GCAGGTCATAGTGATCCGGTGGTTCTGAATGGAAGGGCCATCGCTCAACGGATAAAAGGTA | |
| | CTCCGGGGATAACAGGCTGATACCGCCCAAGAGTTCATATCGACGGCGGTGTTTGGCACCT | |
| | CGATGTCGGCTCATCACATCCTGGGGCTGAAGTAGGTCCCAAGGGTATGGCTGTTCGCCAT | |
| | TTAAAGTGGTACGCGAGCTGGGTTTAGAACGTCGTGAGACAGTTCGGTCCCTATCTGCCGT | |
| | GGGCGCTGGAGAACTGAGGGGGGCTGCTCCTAGTACGAGAGGACCGGAGTGGACGCATCAC | |
| | TGGTGTTCGGGTTGTCATGCCAATGGCACTGCCCGGTAGCTAAATGCGGAAGAGATAAGTG | |
| | CTGAAAGCATCTAAGCACGAAACTTGCCCCGAGATGAGTTCTCCCTGACCCTTTAAGGGTC | |
| | CTGAAGGAACGTTGAAGACGACGACGTTGATAGGCCGGGTGTGTAAGCGCAGCGATGCGTT | |
| | GAGCTAACCGGTACTAATGAACCGTGAGGCTTAACCTTACAACGCCGAAGCTGTTTTGGCG | |
| | GATGAGAGAAGATTTTCAGCCTGATACAGATTAAATCAGAACGCAGAAGCGGTCTGATAAA | |
| | ACAGAATTTGCCTGGCGGCAGTAGCGCGGTGGTCCCACCTGACCCCATGCCGAACTCAGAA | |
| | GTGAAACGCCGTAGCGCCGATGGTAGTGTGGGGTCTCCCCATGCGAGAGTAGGGAACTGCC | |
| | AGGCATCAAATAAAACGAAAGGCTCAGTCGAAAGACTGGGCCTTTCGTTTTATCTGTTGTT | |
| | TGTCGGTGAACGCTCTCCagaaggagattttcaacatgctccctcaatcggttgaatgtcg | |
| | cccttttgtctttggcgctggtaaaccatatgaattttctattgattgtgacaaaataaac | |
| | ttattccgtggtgtctttgcgtttcttttatatgttgccacctttatgtatgtatCttcta | |
| | cgtttgctaacatactgcgtaataaggagtcttaatcatgccagttcttttgggtattccg | |
| | ttattattgcgtttcctcggtttccttctggtaactttgttcggctatctgcttacttttc | |
| | tCaaaaagggcttcggtaagatagctattgctatttcattgtttcttgctcttattattgg | |
| | gcttaactcaattcttgtgggttatctctctgatattagTgctcaattaccctctgacttt | |
| | gttcagggtgttcagttaattctcccgtctaatgcgcttccctgtttttatgttattctct | |
| | ctgtaaagActgctattttcatttttgacgttaaacaaaaaatcgtttcttatttggattg | |
| | ggaCaaataatatggctgtttattttgtaactggcaaattaggctctggaaagacgctcgt | |
| | tagcgttggtaagattcaggataaaattgtagctgggtgcaaaatagcaactaatcttgat | |
| | ttaaggcttcaaaacctcccgcaagtcgggaggttcgctaaaacgcctcgcgttcttagaa | |
| | taccggataagccttctatatctgatttgcttgctattgggcgcggtaatgattcctacga | |
| | tgaaaataaaaacggAttgcttgttctcgatgagtgcggtacttggtttaatacccgttct | |
| | tggaatgataaggaaagacagccgattattgattggtttctacatgctcgtaaattaggat | |
| | gggatattatttttcttgttcaggacttatctattgttgataaacaggcgcgttctgcatt | |
| | agctgaacatgttgtttattgtcgtcgtctggacagaattactttaccttttgtcggtact | |
| | ttatattctcttattactggctcgaaaatgcctctgcctaaattacatgttggcgttgtta | |
| | aatatggcgattctcaattaagccctactgttgagcgttggctttatactggtaagaattt | |
| | gtataacgcataCgatactaaacaggctttttctagtaattatgattccggtgtttattct | |
| | tatttaacgccttatttatcacacggtcggtatttcaaaccattaaatttaggtcagaaga | |
| | tgaaattaactaaaatatatttgaaaaagttttctcgcgttctttgtcttgcgattggatt | |
| | tgcatcagcatttacatatagttatataacccaacctaagccggaggttaaaaaggtagtc | |
| | tctcagacctatgattttgataaattcactattgactcttctcaTcgtcttaatctaagct | |
| | atcgctatgttttcaaggattctaagggaaaattaattaatagcgacgatttacagaagca | |
| | aggttattcactTacatatattgatttatgtactgtttccattaaaaaaggtaattcaaat | |
| | gaaattgttaaatgtaattaattttgttttcttgatgtttgtttcatcatcttcttttgct | |
| | caggtaattgaaatgaataattcgcctctgcgcgattttAtaacttggtattcaaagcaat | |
| | caggcgaatccgttattgtttctcccgatgtaaaaggtactgttactgtatattcatctga | |
| | cgttaaacctgaaaatctacgcaatttctttatttctgttttacgtgcaaataattttgat | |
| | atggtaggttctaacccttccattattcagaagtataatccaaacaatcaggattatattg | |
| | atgaattgccatcaCctgataatcaggaatatgatgataattccgctccttctggtggttt | |
| | ctttgtCccgcaaaatgataatgttactcaaacttttaaaattaataacgttcgggcaaag | |
| | gatttaatacgagttgtcgaattgtttgtaaagtctaatacttctaaatcctcaaatgtat | |
| | tatctattgacggAtctaatctattagttgttagtgctcctaaagatattttagataacct | |
| | tTctcaattcctttcaactgttgatttgccaactgaccagGtattgattgagggtttgata | |
| | tttgaggttcagcaaggtgatgctttagatttttcatttgctgctggctctcagcgtggca | |
| | ctgttgcaggcggAgttaatactgaccgcctcacctctgttttatcttctgctggtggttc | |
| | gttcggtatttttaatggcgatgttttagggctatcagttcgcgcattaaagactaatagc | |
| | cattcaaaaatattgtctgtgccacgtattcttacgctttcaggtcagaagggttctatct | |
| | ctAttggccagaatgtcccttttattactggtcgtgtAactggtgaatctgccaatgtaaa | |
| | taatccatttcagacgattgagcgtcaaaatgtaggtatttccatgagcgtttttcctgtt | |
| | gcaatggctggcggtaatattgttctggatattaccagcaaggccgatagtttgagttctt | |
| | ctactcaggcaagtgatgttattactaaCcaaagaagtattgctacaacggttaatttgcg | |
| | tgatggacagactcttCtactcggtggcctcactgattataaaaacacttctcaggattct | |
| | ggcgtaccgttcctgtctaaaatccctttaatcggcctcctgtttagctcccgctctgatt | |
| | ctaacgaggaGagcacgttatacgtgctcgtcaaagcaaccatagtacgcgccctgtagcg | |
| | gcgcattaagcgcggcgggtgtggtggttacgcgcagcgtgaccgctacacttgccagcgc | |
| | cctagcgcccgctcctttcgctttcttcccttcctttctcgccacgttcgccggctttccc | |
| | cgtcaagcGctaaatcgggggcccctttagggttccgatttagtgctttacggcacctcga | |
| | ccccaaaaaacttgatttgggtgatggttcacgtagtgggccatcgccctgatagacggtt | |
| | tttcgccctttgacgttggagtccacgttctttaatagtggactcttgttccaaactggaa | |
| | caacactcaaccctatctcgggctattcttttgatttataagggattttgccgatttcggc | |
| | ctattggttaaaaaatgaActgatttaacaaaaatttaacgcgaattttaacaaaatatta | |
| | acgtttacaatttaaatatttgcttatacaatcttcctgtCttGggggcttttcttattat | |
| | caaccggggtacat | |
| SP_{PA} | atgattgacatgctagttttacgGttaccgttcatcgattctcttgtttgctccagactct | 86 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattGcagggGca | |
| | taatgtttttggtacaaccgatttagGtttatgTtctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgataCttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaAgggAattcaatgaatatttatgacgat | |
| | tccgcagtattggCcgctatccagtctaaacattttactattaccccctctggcaaaactt | |
| | cttttgcaaaagcctctcgctattttggtttCtatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccctgcaaaaa | |
| | ataaattcatataaaaaacatacagataaccatctgcggtgataaattatctctggcggtg | |
| | ttgacataaatacactggcggttatactgagcacgggtaccGGCCGCTGAGAAAAAGCGAA | |
| | GCGGCACTGCTCTTTAACAATTTATCAGACAATCTGTGTGGGCACTCGAAGATACGGATTC | |
| | TTAACGTCGCAAGACGAAAAATGAATACCAAGTCTCAAGAGTGAACACGTAATTCATTACG | |
| | AAGTTTAATTCTTTGAGCGTCAAACTTTTGAACTGAAGAGTTTGATCATGGCTCAGATTGA | |
| | ACGCTGGCGGCAGGCCTAACACATGCAAGTCGAGCGGATGAAGGGAGCTTGCTCCTGGATT | |
| | CAGCGGCGGACGGGTGAGTAATGCCTAGGAATCTGCCTGGTAGTGGGGGATAACGTCCGGA | |
| | AACGGGCGCTAATACCGCATACGTCCTGAGGGAGAAAGTGGGGGATCTTCGGACCTCACGC | |
| | TATCAGATGAGCCTAGGTCGGATTAGCTAGTTGGTGGGGTAAAGGCCTACCAAGGCGACGA | |
| | TCCGTAACTGGTCTGAGAGGATGATCAGTCACACTGGAACTGAGACACGGTCCAGACTCCT | |
| | ACGGGAGGCAGCAGTGGGGAATATTGGACAATGGGCGAAAGCCTGATCCAGCCATGCCGCG | |
| | TGTGTGAAGAAGGTCTTCGGATTGTAAAGCACTTTAAGTTGGGAGGAAGGGCAGTAAGTTA | |
| | ATACCTTGCTGTTTTGACGTTACCAACAGAATAAGCACCGGCTAACTTCGTGCCAGCAGCC | |
| | GCGGTAATACGAAGGGTGCAAGCGTTAATCGGAATTACTGGGCGTAAAGCGCGCGTAGGTG | |
| | GTTCAGCAAGTTGGATGTGAAATCCCCGGGCTCAACCTGGGAACTGCATCCAAAACTACTG | |
| | AGCTAGAGTACGGTAGAGGGTGGTGGAATTTCCTGTGTAGCGGTGAAATGCGTAGATATAG | |
| | GAAGGAACACCAGTGGCGAAGGCGACCACCTGGACTGATACTGACACTGAGGTGCGAAAGC | |
| | GTGGGGAGCAAACAGGATTAGATACCCTGGTAGTCCACGCCGTAAACGATGTCGACTAGCC | |
| | GTTGGGATCCTTGAGATCTTAGTGGCGCAGCTAACGCGATAAGTCGACCGCCTGGGGAGTA | |
| | CGGCCGCAAGGTTAAAACTCAAATGAATTGACGGGGGCCCGCACAAGCGGTGGAGCATGTG | |
| | GTTTAATTCGAAGCAACGCGAAGAACCTTACCTGGCCTTGACATGCTGAGAACTTTCCAGA | |
| | GATGGATTGGTGCCTTCGGGAACTCAGACACAGGTGCTGCATGGCTGTCGTCAGCTCGTGT | |
| | CGTGAGATGTTGGGTTAAGTCCCGTAACGAGCGCAACCCTTGTCCTTAGTTACCAGCACCT | |
| | CGGGTGGGCACTCTAAGGAGACTGCCGGTGACAAACCGGAGGAAGGTGGGGATGACGTCAA | |
| | GTCATCATGGCCCTTACGGCCAGGGCTACACACGTGCTACAATGGTCGGTACAAAGGGTTG | |
| | CCAAGCCGCGAGGTGGAGCTAATCCCATAAAACCGATCGTAGTCCGGATCGCAGTCTGCAA | |
| | CTCGACTGCGTGAAGTCGGAATCGCTAGTAATCGTGAATCAGAATGTCACGGTGAATACGT | |
| | TCCCGGGCCTTGTACACACCGCCCGTCACACCATGGGAGTGGGTTGCTCCAGAAGTAGCTA | |
| | GTCTAACCGCAAGGGGGACGGTTACCACGGAGTGATTCATGACTGGGGTGAAGTCGTAACA | |
| | AGGTAGCCGTAGGGGAACCTGCGGCTGGATCACTTGTATACCTTAAAGAAGCGTACTTTGT | |
| | AGTGCTCACACAGATTGTCTGATAGAAAGTGAAAAGCAAGGCGTTTACGCGTTGGGAGTGA | |
| | GGCTGAAGAGAATAAGGCCGTTCGCTTTCTATTAATGAAAGCTCACCCTACACGAAAATAT | |
| | CACGCAACGCGTGATAAGCAATTTTCGTGTCCCCTTCGTCTAGAGGCCCAGGACACCGCCC | |
| | TTTCACGGCGGTAACAGGGGTTCGAATCCCCTAGGGGACGCCACTTGCTGGTTTGTGAGTG | |
| | AAAGTCGCCGACCTTAATATCTCAAAACTCATCTTCGGGTGATGTTTGAGATATTTGCTCT | |
| | TTAAAAATCTGGATCAAGCTGAAAATTGAAACACTGAACAACGAGAGTTGTTCGTGAGTCT | |
| | CTCAAATTTTCGCAACACGATGATGAATCGAAAGAAACATCTTCGGGTTGTGAGGTCAAGT | |
| | GAAGAAGCGCATACGGTGGATGCCTTGGCAGTCAGAGGCGATGAAAGACGTGGTAGCCTGC | |
| | GAAAAGCTTCGGGGAGTCGGCAAACAGACTTTGATCCGGAGATCTCTGAATGGGGGAACCC | |
| | ACCTAGGATAACCTAGGTATCTTGTACTGAATCCATAGGTGCAAGAGGCGAACCAGGGGAA | |
| | CTGAAACATCTAAGTACCCTGAGGAAAAGAAATCAACCGAGATTCCCTTAGTAGTGGCGAG | |
| | CGAACGGGGATTAGCCCTTAAGCTTCATTGATTTTAGCGGAACGCTCTGGAAAGTGCGGCC | |
| | ATAGTGGGTGATAGCCCCGTACGCGAAAGGATCTTTGAAGTGAAATCGAGTAGGACGGAGC | |
| | ACGAGAAACTTTGTCTGAACATGGGGGGACCATCCTCCAAGGCTAAATACTACTGACTGAC | |
| | CGATAGTGAACCAGTACCGTGAGGGAAAGGCGAAAAGAACCCCGGAGAGGGGAGTGAAATA | |
| | GAACCTGAAACCGTATGCGTACAAGCAGTGGGAGCCTACTTGTTAGGTGACTGCGTACCTT | |
| | TTGTATAATGGGTCAGCGACTTATATTCAGTGGCAAGCTTAACCGTATAGGGTAGGCGTAG | |
| | CGAAAGCGAGTCTTAATAGGGCGTTTAGTCGCTGGGTATAGACCCGAAACCGGGCGATCTA | |
| | TCCATGAGCAGGTTGAAGGTTAGGTAACACTGACTGGAGGACCGAACCCACTCCCGTTGAA | |
| | AAGGTAGGGGATGACTTGTGGATCGGAGTGAAAGGCTAATCAAGCTCGGAGATAGCTGGTT | |
| | CTCCTCGAAAGCTATTTAGGTAGCGCCTCATGTATCACTCTGGGGGGTAGAGCACTGTTTC | |
| | GGCTAGGGGGTCATCCCGACTTACCAAACCGATGCAAACTCCGAATACCCAGAAGTGCCGA | |
| | GCATGGGAGACACACGGCGGGTGCTAACGTCCGTCGTGAAAAGGGAAACAACCCAGACCGC | |
| | CAGCTAAGGTCCCAAAGTTGTGGTTAAGTGGTAAACGATGTGGGAAGGCTTAGACAGCTAG | |
| | GAGGTTGGCTTAGAAGCAGCCACCCTTTAAAGAAAGCGTAATAGCTCACTAGTCGAGTCGG | |
| | CCTGCGCGGAAGATGTAACGGGGCTCAAACCACACACCGAAGCTGCGGGTGTCACGTAAGT | |
| | GACGCGGTAGAGGAGCGTTCTGTAAGCCTGTGAAGGTGAGTTGAGAAGCTTGCTGGAGGTA | |
| | TCAGAAGTGCGAATGCTGACATGAGTAACGACAATGGGTGTGAAAAACACCCACGCCGAAA | |
| | GACCAAGGGTTCCTGCGCAACGTTAATCGACGCAGGGTTAGTCGGTTCCTAAGGCGAGGCT | |
| | GAAAAGCGTAGTCGATGGGAAACAGGTTAATATTCCTGTACTTCTGGTTACTGCGATGGAG | |
| | GGACGGAGAAGGCTAGGCCAGCTTGGCGTTGGTTGTCCAAGTTTAAGGTGGTAGGCTGAAA | |
| | TCTTAGGTAAATCCGGGGTTTCAAGGCCGAGAGCTGATGACGAGTCGTCTTTTAGATGACG | |
| | AAGTGGTTGATGCCATGCTTCCAAGAAAAGCTTCTAAGCTTCAGGTAACCAGGAACCGTAC | |
| | CCCAAACCGACACAGGTGGTCGGGTAGAGAATACCAAGGCGCTTGAGAGAACTCGGGTGAA | |
| | GGAACTAGGCAAAATGGCACCGTAACTTCGGGAGAAGGTGCGCCGGCTAGGGTGAAGGATT | |
| | TACTCCGTAAGCTCTGGCTGGTCGAAGATACCAGGCCGCTGCGACTGTTTATTAAAAACAC | |
| | AGCACTCTGCAAACACGAAAGTGGACGTATAGGGTGTGACGCCTGCCCGGTGCCGGAAGGT | |
| | TAATTGATGGGGTTAGCGCAAGCGAAGCTCTTGATCGAAGCCCCGGTAAACGGCGGCCGTA | |
| | ACTATAACGGTCCTAAGGTAGCGAAATTCCTTGTCGGGTAAGTTCCGACCTGCACGAATGG | |
| | CGTAACGATGGCGGCGCTGTCTCCACCCGAGACTCAGTGAAATTGAAATCGCTGTGAAGAT | |
| | GCAGTGTATCCGCGGCTAGACGGAAAGACCCCGTGAACCTTTACTGTAGCTTTGCACTGGA | |
| | CTTTGAGCCTGCTTGTGTAGGATAGGTGGGAGGCTTTGAAGCGTGGACGCCAGTTCGCGTG | |
| | GAGCCATCCTTGAAATACCACCCTGGCATGCTTGAGGTTCTAACTCTGGTCCGTAATCCGG | |
| | ATCGAGGACAGTGTATGGTGGGCAGTTTGACTGGGGCGGTCTCCTCCTAAAGAGTAACGGA | |
| | GGAGTACGAAGGTGCGCTCAGACCGGTCGGAAATCGGTCGCAGAGTATAAAGGCAAAAGCG | |
| | CGCTTGACTGCGAGACAGACACGTCGAGCAGGTACGAAAGTAGGTCTTAGTGATCCGGTGG | |
| | TTCTGTATGGAAGGGCCATCGCTCAACGGATAAAAGGTACTCCGGGGATAACAGGCTGATA | |
| | CCGCCCAAGAGTTCATATCGACGGCGGTGTTTGGCACCTCGATGTCGGCTCATCACATCCT | |
| | GGGGCTGAAGCCGGTCCCAAGGGTATGGCTGTTCGCCATTTAAAGTGGTACGCGAGCTGGG | |
| | TTTAGAACGTCGTGAGACAGTTCGGTCCCTATCTGCCGTGGACGTTTGAGATTTGAGAGGG | |
| | GCTGCTCCTAGTACGAGAGGACCGGAGTGGACGAACCTCTGGTGTTCCGGTTGTCACGCCA | |
| | GTGGCATTGCCGGGTAGCTATGTTCGGAAAAGATAACCGCTGAAAGCATCTAAGCGGGAAA | |
| | CTTGCCTCAAGATGAGATCTCACTGGGAACTTGATTCCCCTGAAGGGCCGTCGAAGACTAC | |
| | GACGTTGATAGGCTGGGTGTGTAAGCGTTGTGAGGCGTTGAGCTAACCAGTACTAATTGCC | |
| | CGTGAGGCTTGACCATACAACGCCGAAGCTGTTTTGGCGGATGAGAGAAGATTTTCAGCCT | |
| | GATACAGATTAAATCAGAACGCAGAAGCGGTCTGATAAAACAGAATTTTGCTTGACGATCA | |
| | TAGAGCGTTGGAACCACCTGATCCCTTCCCGAACTCAGAAGTGAAACGACGCATCGCCGAT | |
| | GGTAGTGTGGGGTCTCCCCATGTGAGAGTAGGTCATCGTCAAGCTCCAAATAAAACGAAAG | |
| | GCTCAGTCGAAAGACTGGGCCTTTCGTTTTATCTGTTGTTTGTCGGTGAACGCTCTCCTGA | |
| | GTAGGACAAATCCGCCGGGAGCGGATTTGAACGTTGCGAAGCAACGGCCCGGAGGGTGGCG | |
| | GGCAGGACGCCCGCCATAAACTGCCAGGCATCAAATTAAGCAGAAGGCCATCCTGACGGAT | |
| | GGCCTTTTTGCGTTTCTACAAACTCTTCCTGTCGTCATATCTACAAGCCagaaggagattt | |
| | tcaacatgctccctcaatcggttgaatgtcgcccttttgtctttggcgctggtaaaccata | |
| | tgaattttctattgattgtgacaaaataaacttattccgtggtgtctttgcgtttctttta | |
| | tatgttgccacctttatgtatgtatCttctacgtttgctaacatactgcgtaataaggagt | |
| | cttaatcatgccagttcttttgggtattccgttattattgcgtttcctcggtttccttctg | |
| | gtaactttgttcggctatctgcttacttttctCaaaaagggcttcggtaagatagctattg | |
| | ctatttcattgtttcttgctcttattattgggcttaactcaattcttgtgggttatctctc | |
| | tgatattagTgctcaattaccctctgactttgttcagggtgttcagttaattctcccgtct | |
| | aatgcgcttccctgtttttatgttattctctctgtaaagActgctattttcatttttgacg | |
| | ttaaacaaaaaatcgtttcttatttggattgggaCaaataatatggctgtttattttgtaa | |
| | ctggcaaattaggctctggaaagacgctcgttagcgttggtaagattcaggataaaattgt | |
| | agctgggtgcaaaatagcaactaatcttgatttaaggcttcaaaacctcccgcaagtcggg | |
| | aggttcgctaaaacgcctcgcgttcttagaataccggataagccttctatatctgatttgc | |
| | ttgctattgggcgcggtaatgattcctacgatgaaaataaaaacggAttgcttgttctcga | |
| | tgagtgcggtacttggtttaatacccgttcttggaatgataaggaaagacagccgattatt | |
| | gattggtttTtacatgctcgtaaattaggatgggatattatttttcttgttcaggacttat | |
| | cCattgttgataaacaggcgcgttctgcattagctgaacatgttgtttattgtcgtcgtct | |
| | ggacagaattactttaccttttgtcggtactttatattctcttattactggctcgaaaatg | |
| | cctctgcctaaattacatgttggcgttgttaaatatggcgattctcaattaagcccCactg | |
| | ttgagcgttggctttatactggtaagaatttgtataacgcataCgatactaaacaggcttt | |
| | ttctagtaattatgattccggtgtttattcttatttaacgccttatttatcacacggtcgg | |
| | tatttcaaaccattaaatttaggtcagaagatgaaattaactaaaatatatttgaaaaagt | |
| | tttctcgcgttctttgtcttgcgattggatttgcatcagcatttacatatagttatataac | |
| | ccaacctaagccggaggttaaaaaggtagtctctcagacctatgattttgataaattcact | |
| | attgactcttctcaTcgtcttaatctaagctatcgctatgttttcaaggattctaagggaa | |
| | aattaattaatagcgacgatttacagaagcaaggttattcactTacatatattgatttatg | |
| | tactgtttccattaaaaaaggtaattcaaatgaaattgttaaatgtaattaattCtgtttt | |
| | cttgatgtttgtttcatcatcttcttttgctcaggtaattgaaatgaataattcgcctctg | |
| | cgcgattttAtaacttggtattcaaagcaatcaggcgaatccgttattgtttctcccgatg | |
| | taaaaggtactgttactgtatattcatctgacgttaaacctgaaaatctacgcaatttctt | |
| | tatttctgttttacgtgcaaataattttgatatggtaggttctaacccttccattattcag | |
| | aagtataatccaaacaatcaggattatattgatgaattgccatcaCctgataatcaggaat | |
| | atgatgataattccgctccttctggtggtttctttgtCccgcaaaatgataatgttactca | |
| | aacttttaaaattaataacgttcgggcaaaggatttaatacgagttgtcgaattgtttgta | |
| | aagtctaatacttctaaatcctcaaatgtattatctattgacggAtctaatctattagttg | |
| | ttagtgctcctaaagatattttagataaccttTctcaattcctttcaactgttgatttgcc | |
| | aactgaccagGtattgattgagggtttgatatttgaggttcagcaaggtgatgctttagat | |
| | ttttcatttgctgctggctctcagcgtggcactgttgcaggcggAgttaatactgaccgcc | |
| | tcacctctgttttatcttctgctggtggttcgttcggtatttttaatggcgatgttttagg | |
| | gctatcagttcgcgcattaaagactaatagccattcaaaaatattgtctgtgccacgtatt | |
| | cttacgctttcaggtcagaagggttctatctctAttggccagaatgtcccttttattactg | |
| | gtcgtgtAactggtgaatctgccaatgtaaataatccatttcagacgattgagcgtcaaaa | |
| | tgtaggtatttccatgagcgtttttcctgttgcaatggctggcggtaatattgttctggat | |
| | attaccagcaaggccgatagtttgagttcttctactcaggcaagtgatgttattactaaCc | |
| | aaagaagtattgctacaacggttaatttgcgtgatggacagactcttCtactcggtggcct | |
| | cactgattataaaaacacttctcaggattctggcgtaccgttcctgtctaaaatcccttta | |
| | atcggcctcctgtttagctcccgctctgattctaacgaggaGagcacgttatacgtgctcg | |
| | tcaaagcaaccatagtacgcgccctgtagcggcgcattaagcgcggcgggtgtggtggtta | |
| | cgcgcagcgtgaccgctacacttgccagcgccctagcgcccgctcctttcgctttcttccc | |
| | ttcctttctcgccacgttcgccggctttccccgtcaagcGctaaatcgggggcccctttag | |
| | ggttccgatttagtgctttacggcacctcgaccccaaaaaacttgatttgggtgatggttc | |
| | acgtagtgggccatcgccctgatagacggtttttcgccctttgacgttggagtccacgttc | |
| | ttgatttataagggattttgccgGtttcggcctattggttaaaaaatgaActgatttaaca | |
| | aaaatttaacgcgaattttaacaaaatattaacgtttacaatttaaatatttgcttataca | |
| | atcttcctgtCttGggggcttttcttattatcaaccggggtacat | |
| SP_{VC} | atgattgacatgctagttttacgGttaccgttcatcgattctcttgtttgctccagactct | 87 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccccctgcaaa | |
| | aaataaattcatataaaaaacatacagataaccatctgcggtgataaattatctctggcgg | |
| | tgttgacataaataccactggcggttatactgagcacgggtaccGGCCGCTGAGAAAAAGC | |
| | GAAGCGGCACTGCTCACTGCTCTTTAACAATTTATCAGACAATCTGTGTGGGCACTCGAAG | |
| | ATACGGATTCTTAACGTCGCAAGACGAAAAATGAATACCAAGTCTCAAGAGTGAACACGTA | |
| | ATTCATTACGAAGTTTAATTCTTTGAGCGTCAAACTTTTTAATTGAAGAGTTTGATCATGG | |
| | CTCAGATTGAACGCTGGCGGCAGGCCTAACACATGCAAGTCGAGCGGCAGCACAGAGGAAC | |
| | TTGTTCCTTGGGTGGCGAGCGGCGGACGGGTGAGTAATGCCTGGGAAATTGCCCGGTAGAG | |
| | GGGGATAACCATTGGAAACGATGGCTAATACCGCATAACCTCGCAAGAGCAAAGCAGGGGA | |
| | CCTTCGGGCCTTGCGCTACCGGATATGCCCAGGTGGGATTAGCTAGTTGGTGAGGTAAGGG | |
| | CTCACCAAGGCGACGATCCCTAGCTGGTCTGAGAGGATGATCAGCCACACTGGAACTGAGA | |
| | CACGGTCCAGACTCCTACGGGAGGCAGCAGTGGGGAATATTGCACAATGGGCGCAAGCCTG | |
| | ATGCAGCCATGCCGCGTGTATGAAGAAGGCCTTCGGGTTGTAAAGTACTTTCAGTAGGGAG | |
| | GAAGGTGGTTAAGTTAATACCTTAATCATTTGACGTTACCTACAGAAGAAGCACCGGCTAA | |
| | CTCCGTGCCAGCAGCCGCGGTAATACGGAGGGTGCAAGCGTTAATCGGAATTACTGGGCGT | |
| | AAAGCGCATGCAGGTGGTTTGTTAAGTCAGATGTGAAAGCCCTGGGCTCAACCTAGGAATC | |
| | GCATTTGAAACTGACAAGCTAGAGTACTGTAGAGGGGGGTAGAATTTCAGGTGTAGCGGTG | |
| | AAATGCGTAGAGATCTGAAGGAATACCGGTGGCGAAGGCGGCCCCCTGGACAGATACTGAC | |
| | ACTCAGATGCGAAAGCGTGGGGAGCAAACAGGATTAGATACCCTGGTAGTCCACGCCGTAA | |
| | ACGATGTCTACTTGGAGGTTGTGACCTAGAGTCGTGGCTTTCGGAGCTAACGCGTTAAGTA | |
| | GACCGCCTGGGGAGTACGGTCGCAAGATTAAAACTCAAATGAATTGACGGGGGCCCGCACA | |
| | AGCGGTGGAGCATGTGGTTTAATTCGATGCAACGCGAAGAACCTTACCTACTCTTGACATC | |
| | CAGAGAATCTAGCGGAGACGCTGGAGTGCCTTCGGGAGCTCTGAGACAGGTGCTGCATGGC | |
| | TGTCGTCAGCTCGTGTTGTGAAATGTTGGGTTAAGTCCCGCAACGAGCGCAACCCTTATCC | |
| | TTGTTTGCCAGCACGTAATGGTGGGAACTCCAGGGAGACTGCCGGTGATAAACCGGAGGAA | |
| | GGTGGGGACGACGTCAAGTCATCATGGCCCTTACGAGTAGGGCTACACACGTGCTACAATG | |
| | GCGTATACAGAGGGCAGCGATACCGCGAGGTGGAGCGAATCTCACAAAGTACGTCGTAGTC | |
| | CGGATTGGAGTCTGCAACTCGACTCCATGAAGTCGGAATCGCTAGTAATCGCAAATCAGAA | |
| | TGTTGCGGTGAATACGTTCCCGGGCCTTGTACACACCGCCCGTCACACCATGGGAGTGGGC | |
| | TGCAAAAGAAGCAGGTAGTTTAACCTTCGGGAGGACGCTTGCCACTTTGTGGTTCATGACT | |
| | GGGGTGAAGTCGTAACAAGGTAGCGCTAGGGGAACCTGGCGCTGGATCACTTGTATACCTT | |
| | AAAGAAGCGTACTTTGTAGTGCTCACACAGATTGTCTGATAGAAAGTGAAAAGCAAGGCGT | |
| | TTACGCGTTGGGAGTGAGGCTGAAGAGAATAAGGCCGTTCGCTTTCTATTAATGAAAGCTC | |
| | ACCCTACACGAAAATATCACGCAACGCGTGATAAGCAATTTTCGTGTCCCCTTCGTCTAGA | |
| | GGCCCAGGACACCGCCCTTTCACGGCGGTAACAGGGGTTCGAATCCCCTAGGGGACGCCAC | |
| | TTGCTGGTTTGTGAGTGAAAGTCGCCGACCTTAATATCTCAAAACTCATCTTCGGGTGATG | |
| | TTTGAGATATTTGCTCTTTAAAAATCTGGATCAAGCTGAAAATTGAAACACTGAACAACGA | |
| | GAGTTGTTCGTGAGTCTCTCAAATTTTCGCAACACGATGATGAATCGAAAGAAACATCTTC | |
| | GGGTTGTGAGGTTAAGTGACTAAGCGTACACGGTGGATGCCTGGGCAGTCAGAGGCGATGA | |
| | AGGACGTACTAACTTGCGATAAGCGCAGATAAGGCAGTAAGAGCCGTTTGAGTCTGCGATT | |
| | TCCGAATGGGGAAACCCAACTGCATAAGCAGTTACTGTTAACTGAATACATAGGTTAACAG | |
| | AGCAAACCGGGGGAACTGAAACATCTAAGTACCCCGAGGAGAAGAAATCAACCGAGATTCC | |
| | GGTAGTAGCGGCGAGCGAACCTGGATTAGCCCTTAAGCACTCGGTGAAGTAGGTGAACAAG | |
| | CTGGAAAGCTTGGCGATACAGGGTGATAGCCCCGTAACCGACGCTTCATCGAGCGTGAAAT | |
| | CGAGTAGGGCGGGACACGTGATATCCTGTCTGAATATGGGGGGACCATCCTCCAAGGCTAA | |
| | ATACTCCTGACTGACCGATAGTGAACCAGTACCGTGAGGGAAAGGCGAAAAGAACCCCTGT | |
| | GAGGGGAGTGAAATAGAACCTGAAACCGTGTACGTACAAGCAGTAGGAGCACCTTCGTGGT | |
| | GTGACTGCGTACCTTTTGTATAATGGGTCAGCGACTTATATTCAGTGGCAAGGTTAACCGT | |
| | ATAGGGGAGCCGTAGCGAAAGCGAGTCTTAATTGGGCGCTCAGTCTCTGGATATAGACCCG | |
| | AAACCGGGTGATCTAGCCATGGGCAGGTTGAAGGTTGAGTAACATCAACTGGAGGACCGAA | |
| | CCGACTAATGTTGAAAAATTAGCGGATGACTTGTGGCTAGGGGTGAAAGGCCAATCAAACT | |
| | CGGAGATAGCTGGTTCTCCCCGAAAGCTATTTAGGTAGCGCCTCGGACGAATACTACTGGG | |
| | GGTAGAGCACTGTTAAGGCTAGGGGGTCATCCCGACTTACCAACCCTTTGCAAACTCCGAA | |
| | TACCAGTAAGTACTATCCGGGAGACACACGGCGGGTGCTAACGTCCGTCGTGGAGAGGGAA | |
| | ACAACCCAGACCGCCAGCTAAGGTCCCAAAGTATTGCTAAGTGGGAAACGATGTGGGAAGG | |
| | CTCAGACAGCTAGGATGTTGGCTTAGAAGCAGCCATCATTTAAAGAAAGCGTAATAGCTCA | |
| | CTAGTCGAGTCGGCCTGCGCGGAAGATGTAACGGGGCTAAGCAATACACCGAAGCTGCGGC | |
| | AATGTCTTTTAGATATTGGGTAGGGGAGCGTTCTGTAAGCCGTTGAAGGTGAATCGTAAGG | |
| | TTTGCTGGAGGTATCAGAAGTGCGAATGCTGACATGAGTAACGACAAAGGGGGTGAAAAAC | |
| | CTCCTCGCCGGAAGACCAAGGGTTCCTGTCCAACGTTAATCGGGGCAGGGTGAGTCGACCC | |
| | CTAAGGTGAGGCCGAAAGGCGTAATCGATGGGAAACGGGTTAATATTCCCGTACTTCTGAC | |
| | TATTGCGATGGGGGGACGGAGAAGGCTAGGTGGGCCAGGCGACGGTTGTCCTGGTTCAAGT | |
| | GCGTAGGCTTGAGAGTTAGGTAAATCCGGCTCTCTTTAAGGCTGAGACACGACGTCGAGCT | |
| | GCTACGGCAGTGAAGTCATTGATGCCATGCTTCCAGGAAAAGCCTCTAAGCTTCAGATAGT | |
| | CAGGAATCGTACCCCAAACCGACACAGGTGGTCGGGTAGAGAATACCAAGGCGCTTGAGAG | |
| | AACTCGGGTGAAGGAACTAGGCAAAATGGTACCGTAACTTCGGGAGAAGGTACGCTCTTGA | |
| | TGGTGAAGTCCCTCGCGGATGGAGCTGACGAGAGTCGCAGATACCAGGTGGCTGCAACTGT | |
| | TTATTAAAAACACAGCACTGTGCAAAATCGCAAGATGACGTATACGGTGTGACGCCTGCCC | |
| | GGTGCCGGAAGGTTAATTGATGGGGTTAGCGCAAGCGAAGCTCTTGATCGAAGCCCCGGTA | |
| | AACGGCGGCCGTAACTATAACGGTCCTAAGGTAGCGAAATTCCTTGTCGGGTAAGTTCCGA | |
| | CCTGCACGAATGGCGTAATGATGGCCACGCTGTCTCCACCCGAGACTCAGTGAAATTGAAA | |
| | TCGCTGTGAAGATGCAGTGTACCCGCGGCTAGACGGAAAGACCCCGTGAACCTTTACTACA | |
| | GCTTGGCACTGAACATTGAACCTACATGTGTAGGATAGGTGGGAGTCTATGAAGACGTGAC | |
| | GCCAGTTGCGTTGGAGCCGTCCTTGAAATACCACCCTTGTATGTTTGATGTTCTAACGTTG | |
| | GCCCCTAATCGGGGTTGCGGACAGTGCCTGGTGGGTAGTTTGACTGGGGCGGTCTCCTCCC | |
| | AAAGAGTAACGGAGGAGCACGAAGGTGGGCTAATCACGGTTGGACATCGTGAGGTTAGTGC | |
| | AATGGCATAAGCCCGCTTAACTGCGAGAATGACGGTTCGAGCAGGTGCGAAAGCAGGTCAT | |
| | AGTGATCCGGTGGTTCTGTATGGAAGGGCCATCGCTCAACGGATAAAAGGTACTCCGGGGA | |
| | TAACAGGCTGATACCGCCCAAGAGTTCATATCGACGGCGGTGTTTGGCACCTCGATGTCGG | |
| | CTCATCACATCCTGGGGCTGAAGTCGGTCCCAAGGGTATGGCTGTTCGCCATTTAAAGTGG | |
| | TACGCGAGCTGGGTTTAGAACGTCGTGAGACAGTTCGGTCCCTATCTGCCGTGGGCGTTGG | |
| | AAGATTGAAGGGGGCTGCTCCTAGTACGAGAGGACCGGAGTGGACGAACCTCTGGTGTTCG | |
| | GGTTGTGTCGCCAGACGCATTGCCCGGTAGCTAAGTTCGGAATTGATAAGCGCTGAAAGCA | |
| | TCTAAGCGCGAAGCGAGCCCTGAGATGAGTCTTCCCTGACGGTTTAACCGTCCTAAAGGGT | |
| | TGTTCGAGACTAGAACGTTGATAGGCAGGGTGTGTAAGCGTTGTGAGGCGTTGAGCTAACC | |
| | TGTACTAATTGCCCGTGAGGCTTAACCATACAACGCCGAAGCTGTTTTGGCGGATGAGAGA | |
| | AGATTTTCAGCCTGATACAGATTAAATCAGAACGCAGAAGCGGTCTGATAAAACAGAATTT | |
| | TGCTTGGCGACCATAGCGTTTTGGACCCACCTGACTCCATCCCGAACTCAGAAGTGAAACG | |
| | AAACAGCGTCGATGGTAGTGTGGGGTCTCCCCATGTGAGAGTAGAACATCGCCAGGCTTCA | |
| | AATAAAACGAAAGGCTCAGTCGAAAGACTGGGCCTTTCGTTTTATCTGTTGTTTGTCGGTG | |
| | AACGCTCTCCTGAGTAGGACAAATCCGCCGGGAGCGGATTTGAACGTTGCGAAGCAACGGC | |
| | CCGGAGGGTGGCGGGCAGGACGCCCGCCATAAACTGCCAGGCATCAAATTAAGCAGAAGGC | |
| | CATCCTGACGGATGGCCTTTTTGCGTTTCTACAAACTCTTCCTGTCGTCATATCTACAAGC | |
| | Cagaaggagattttcaacatgctccctcaatcggttgaatgtcgcccttttgtctttggcg | |
| | ctggtaaaccatatgaattttctattgattgtgacaaaataaacttattccgtggtgtctt | |
| | tgcgtttcttttatatgttgccacctttatgtatgtatCttctacgtttgctaacatactg | |
| | cgtaataaggagtcttaatcatgccagttcttttgggtattccgttattattgcgtttcct | |
| | cggtttccttctggtaactttgttcggctatctgcttacttttctCaaaaagggcttcggt | |
| | aagatagctattgctatttcattgtttcttgctcttattattgggcttaactcaattcttg | |
| | tgggttatctctctgatattagTgctcaattaccctctgactttgttcagggtgttcagtt | |
| | aattctcccgtctaatgcgcttccctgtttttatgttattctctctgtaaagActgctatt | |
| | ttcatttttgacgttaaacaaaaaatcgtttcttatttggattgggaCaaataatatggct | |
| | gtttattttgtaactggcaaattaggctctggaaagacgctcgttagcgttggtaagattc | |
| | aggataaaattgtagctgggtgcaaaatagcaactaatcttgatttaaggcttcaaaacct | |
| | cccgcaagtcgggaggttcgctaaaacgcctcgcgttcttagaataccggataagccttct | |
| | atatctgatttgcttgctattgggcgcggtaatgattcctacgatgaaaataaaaacggAt | |
| | tgcttgttctcgatgagtgcggtacttggtttaatacccgttcttggaatgataaggaaag | |
| | acagccgattattgattggtttctacatgctcgtaaattaggatgggatattatttttctt | |
| | gttcaggacttatctattgttgataaacaggcgcgttctgcattagctgaacatgttgttt | |
| | attgtcgtcgtctggacagaattactttaccttttgtcggtactttatattctcttattac | |
| | tggctcgaaaatgcctctgcctaaattacatgttggcgttgttaaatatggcgattctcaa | |
| | ttaagccctactgttgagcgttggctttatactggtaagaatttgtataacgcataCgata | |
| | ctaaacaggctttttctagtaattatgattccggtgtttattcttatttaacgccttattt | |
| | atcacacggtcggtatttcaaaccattaaatttaggtcagaagatgaaattaactaaaata | |
| | tatttgaaaaagttttctcgcgttctttgtcttgcgattggatttgcatcagcatttacat | |
| | atagttatataacccaacctaagccggaggttaaaaaggtagtctctcagacctatgattt | |
| | tgataaattcactattgactcttctcaTcgtcttaatctaagctatcgctatgttttcaag | |
| | gattctaagggaaaattaattaatagcgacgatttacagaagcaaggttattcactTacat | |
| | atattgatttatgtactgtttccattaaaaaaggtaattcaaatgaaattgttaaatgtaa | |
| | ttaattttgttttcttgatgtttgtttcatcatcttcttttgctcaggtaattgaaatgaa | |
| | taattcgcctctgcgcgattttAtaacttggtattcaaagcaatcaggcgaatccgttatt | |
| | gtttctcccgatgtaaaaggtactgttactgtatattcatctgacgttaaacctgaaaatc | |
| | tacgcaatttctttatttctgttttacgtgcaaataattttgatatggtaggttctaaccc | |
| | ttccattattcagaagtataatccaaacaatcaggattatattgatgaattgccatcaCct | |
| | gataatcaggaatatgatgataattccgctccttctggtggtttctttgtCccgcaaaatg | |
| | ataatgttactcaaacttttaaaattaataacgttcgggcaaaggatttaatacgagttgt | |
| | cgaattgtttgtaaagtctaatacttctaaatcctcaaatgtattatctattgacggAtct | |
| | aatctattagttgttagtgctcctaaagatattttagataaccttTctcaattcctttcaa | |
| | ctgttgatttgccaactgaccagGtattgattgagggtttgatatttgaggttcagcaagg | |
| | tgatgctttagatttttcatttgctgctggctctcagcgtggcactgttgcaggcggAgtt | |
| | aatactgaccgcctcacctctgttttatcttctgctggtggttcgttcggtatttttaatg | |
| | gcgatgttttagggctatcagttcgcgcattaaagactaatagccattcaaaaatattgtc | |
| | tgtgccacgtattcttacgctttcaggtcagaagggttctatctctAttggccagaatgtc | |
| | ccttttattactggtcgtgtAactggtgaatctgccaatgtaaataatccatttcagacga | |
| | ttgagcgtcaaaatgtaggtatttccatgagcgtttttcctgttgcaatggctggcggtaa | |
| | tattgttctggatattaccagcaaggccgatagtttgagttcttctactcaggcaagtgat | |
| | gttattactaaCcaaagaagtattgctacaacggttaatttgcgtgatggacagactcttC | |
| | tactcggtggcctcactgattataaaaacacttctcaggattctggcgtaccgttcctgtc | |
| | taaaatccctttaatcggcctcctgtttagctcccgctctgattctaacgaggaGagcacg | |
| | ttatacgtgctcgtcaaagcaaccatagtacgcgccctgtagcggcgcattaagcgcggcg | |
| | ggtgtggtggttacgcgcagcgtgaccgctacacttgccagcgccctagcgcccgctcctt | |
| | tcgctttcttcccttcctttctcgccacgttcgccggctttccccgtcaagcGctaaatcg | |
| | ggggcccctttagggttccgatttagtgctttacggcacctcgaccccaaaaaacttgatt | |
| | tgggtgatggttcacgtagtgggccatcgccctgatagacggtttttcgccctttgacgtt | |
| | ggagtccacgttctttaatagtggactcttgttccaaactggaacaacactcaaccctatc | |
| | tcgggctattcttttgatttataagggattttgccgatttcggcctattggttaaaaaatg | |
| | aActgatttaacaaaaatttaacgcgaattttaacaaaatattaacgtttacaatttaaat | |
| | atttgcttatacaatcttcctgtCttGggggcttttcttattatcaaccggggtacat | |
| AP1 | AACACCCCTTGTATTACTGTTTATGTAAGCAGACAGTTTTATTGTTCATGATGATATATTT | 88 |
| | TTATCTTGTGCAATGTAACATCAGAGATTTTGAGACACAACGTGGCTTTCCATCAAAAAAA | |
| | TATTGACAACATAAAAAACTTTGTGTTATACTTGTGGAATTGTGAGCGGATAACAATTCTA | |
| | TATCTGTTATTTTTTCCAACCACAGATCTatgaaaaaattattattcgcaattcctttagt | |
| | tgttcctttctattctcactccgctgaaactgttgaaagttgtttagcaaaaccccataca | |
| | gaaaattcatttactaacgtctggaaagacgacaaaactttagatcgttacgctaactatg | |
| | agggctgtctgtggaatgctacaggcgttgtagtttgtactggtgacgaaactcagtgtta | |
| | cggtacatgggttcctattgggcttgctatccctgaaaatgagggtggtggctctgagggt | |
| | ggtggttctgagggtggcggttctgagggtggcggtactaaacctcctgagtacggtgata | |
| | cacctattccgggctatacttatatcaaccctctcgacggcacttatccgcctggtactga | |
| | gcaaaaccccgctaatcctaatccttctcttgaggagtctcagcctcttaatactttcatg | |
| | tttcagaataataggttccgaaataggcagggggcattaactgtttatacgggcactgtta | |
| | ctcaaggcactgaccccgttaaaacttattaccagtacactcctgtatcatcaaaagccat | |
| | gtatgacgcttactggaacggtaaattcagagactgcgctttccattctggctttaatgag | |
| | gatccattcgtttgtgaatatcaaggccaatcgtctgacctgcctcaacctcctgtcaatg | |
| | ctggcggcggctctggtggtggttctggtggcggctctgagggtggtggctctgagggtgg | |
| | cggttctgagggtggcggctctgagggaggcggttccggtggtggctctggttccggtgat | |
| | tttgattatgaaaagatggcaaacgctaataagggggctatgaccgaaaatgccgatgaaa | |
| | acgcgctacagtctgacgctaaaggcaaacttgattctgtcgctactgattacggtgctgc | |
| | tatcgatggtttcattggtgacgtttccggccttgctaatggtaatggtgctactggtgat | |
| | tttgctggctctaattcccaaatggctcaagtcggtgacggtgataattcacctttaatga | |
| | ataatttccgtcaatatttaccttccctccctcaatcggttgaatgtcgcccttttgtctt | |
| | tggcgctggtaaaccttacgagttcagtatcgactgcgataagatcaacctgttccgcggt | |
| | gtctttgcgtttcttttatatgttgccacctttatgtatgtattttctacgtttgctaaca | |
| | tactgcgtaataaggagtcttaaGTCGACCGGCTGCTAACAAAGCCCGCGGCCGCTGAAGA | |
| | TCGATCTCGACGAGTGAGAGAAGATTTTCAGCCTGATACAGATTAAATCAGAACGCAGAAG | |
| | CGGTCTGATAAAACAGAATTTGCCTGGCGGCAGTAGCGCGGTGGTCCCACCTGACCCCATG | |
| | CCGAACTCAGAAGTGAAACGCCGTAGCGCCGATGGTAGTGTGGGGTCACCCCATGCGAGAG | |
| | TAGGGAACTGCCAGGCATCAAATAAAACGAAAGGCTCAGTCGAAAGACTGGGCCTTTCGTT | |
| | TTATCTGTTGTTTGTCGGTGAACGCTCTCCTGAGTAGGACAAATCCGCCGGGAGCGGATTT | |
| | GAACGTTGCGAAGCAACGGCCCGGAGGGTGGCGGGCAGGACGCCCGCCATAAACTGCCAGG | |
| | CATCAAATTAAGCAGAAGGCCATCCTGACGGATGGCCTTTTTGCGTTTCTACAGAGCGTCA | |
| | GACCCCTTAATAAGATGATCTTCTTGAGATCGTTTTGGTCTGCGCGTAATCTCTTGCTCTG | |
| | AAAACGAAAAAACCGCCTTGCAGGGCGGTTTTTCGAAGGTTCTCTGAGCTACCAACTCTTT | |
| | GAACCGAGGTAACTGGCTTGGAGGAGCGCAGTCACCAAAACTTGTCCTTTCAGTTTAGCCT | |
| | TAACCGGCGCATGACTTCAAGACTAACTCCTCTAAATCAATTACCAGTGGCTGCTGCCAGT | |
| | GGTGCTTTTGCATGTCTTTCCGGGTTGGACTCAAGACGATAGTTACCGGATAAGGCGCAGC | |
| | GGTCGGACTGAACGGGGGGTTCGTGCATACAGTCCAGCTTGGAGCGAACTGCCTACCCGGA | |
| | ACTGAGTGTCAGGCGTGGAATGAGACAAACGCGGCCATAACAGCGGAATGACACCGGTAAA | |
| | CCGAAAGGCAGGAACAGGAGAGCGCACGAGGGAGCCGCCAGGGGGAAACGCCTGGTATCTT | |
| | TATAGTCCTGTCGGGTTTCGCCACCACTGATTTGAGCGTCAGATTTCGTGATGCTTGTCAG | |
| | GGGGGCGGAGCCTATGGAAAAACGGCTTTGCCGCGGCCCTCTCGGATCTGTATGGTGCACT | |
| | CTCAGTACAATCTGCTCTGATGCCGCATAGTTAAGCCAGTATACACTCCGCTATCGCTACG | |
| | TGACTGCCTCGACCTGCAGCAATTCCAACGCCATCAAAAATAATTCGCGTCTGGCCTTCCT | |
| | GTAGCCAGCTTTCATCAACATTAAATGTGAGCGAGTAACAACCCGTCGGATTCTCCGTGGG | |
| | AACAAACGGCGGATTGACCGTAATGGGATAGGTCACGTTGGTGTAGATGGGCGCATCGTAA | |
| | CCGTGCATCTGCCAGTTTGAGGGGACGACGACAGTATCGGCCTCAGGAAGATCGCACTCCA | |
| | GCCAGCTTTCCGGCACCGCTTCTGGTGCCGGAAACCAGGCAAAGCGCCATTCGCCATTCAG | |
| | GCTGCGCAACTGTTGGGAAGGGCGATCGGTGCGGGCCTCTTCGCTATTACGCCAGCTGGCG | |
| | AAAGGGGGATGTGCTGCAAGGCGATTAAGTTGGGTAACGCCAGGGTTTTCCCAGTCACGAC | |
| | GTTGTAAAACGACGGCCAGTGAATCCGTAATCATGGTCATAGCTGTTTCCTGTGTGAAATT | |
| | GTTATCCGCTCACAATTCCACACAACATACGAGCCGGAAGCATAAAGTGTAAAGCCTGGGG | |
| | TGCCTAATGAGTGAGCTAACTTACATTAATTGCGTTGCGCTCACTGCCCGCTTTCCAGTCG | |
| | GGAAACCTGTCGTGCCAGCTGCATTAATGAATCGGCCAACGCGCGGGGAGAGGCGGTTTGC | |
| | GTATTGGGCGCCAGGGTGGTTTTTCTTTTCACCAGTGAGACGGGCAACAGCTGATTGCCCT | |
| | TCACCGCCTGGCCCTGAGAGAGTTGCAGCAAGCGGTCCACGCTGGTTTGCCCCAGCAGGCG | |
| | AAAATCCTGTTTGATGGTGGTTAACGGCGGGATATAACATGAGCTGTCTTCGGTATCGTCG | |
| | TATCCCACTACCGAGATATCCGCACCAACGCGCAGCCCGGACTCGGTAATGGCGCGCATTG | |
| | CGCCCAGCGCCATCTGATCGTTGGCAACCAGCATCGCAGTGGGAACGATGCCCTCATTCAG | |
| | CATTTGCATGGTTTGTTGAAAACCGGACATGGCACTCCAGTCGCCTTCCCGTTCCGCTATC | |
| | GGCTGAATTTGATTGCGAGTGAGATATTTATGCCAGCCAGCCAGACGCAGACGCGCCGAGA | |
| | CAGAACTTAATGGGCCCGCTAACAGCGCGATTTGCTGGTGACCCAATGCGACCAGATGCTC | |
| | CACGCCCAGTCGCGTACCGTCTTCATGGGAGAAAATAATACTGTTGATGGGTGTCTGGTCA | |
| | GAGACATCAAGAAATAACGCCGGAACATTAGTGCAGGCAGCTTCCACAGCAATGGCATCCT | |
| | GGTCATCCAGCGGATAGTTAATGATCAGCCCACTGACGCGTTGCGCGAGAAGATTGTGCAC | |
| | CGCCGCTTTACAGGCTTCGACGCCGCTTCGTTCTACCATCGACACCACCACGCTGGCACCC | |
| | AGTTGATCGGCGCGAGATTTAATCGCCGCGACAATTTGCGACGGCGCGTGCAGGGCCAGAC | |
| | TGGAGGTGGCAACGCCAATCAGCAACGACTGTTTGCCCGCCAGTTGTTGTGCCACGCGGTT | |
| | GGGAATGTAATTCAGCTCCGCCATCGCCGCTTCCACTTTTTCCCGCGTTTTCGCAGAAACG | |
| | TGGCTGGCCTGGTTCACCACGCGGGAAACGGTCTGATAAGAGACACCGGCATACTCTGCGA | |
| | CATCGTATAACGTTACTGGTTTCACATTCACCACCCTGAATTGACTCTCTTCCGGGCGCTA | |
| | TCATGCCATACCGCGAAAGGTTTTGCACCATTCCATGGTGTCGGAATTGCTGCAGGTCGAG | |
| | GGGGTCATGGCTGCGCCCCGACACCCGCCAACACCCGCTGACGCGCCCTGACGGGCTTGTC | |
| | TGCTCCCGGCATCCGCTTACAGACAAGCTGTGACCGTCTCCGGGAGCTGCATGTGTCAGAG | |
| | GTTTTCACCGTCATCACCGAAACGCGCGAGGCAGGATGGCGCCCAACAGTCCCCCGGCCAC | |
| | GGGGCCTGCCACCATACCCACGCCGAAACAAGCGCTCATGAGCCCGAAGTGGCGAGCCCGA | |
| | TCTTCCCCATCGGTGATGTCGGCGATATAGGCGCCAGCAACCGCACCTGTGGCGCCGGTGA | |
| | TGCCGGCCACGATGCGTCCGGCGTAGAGGATCCGTCGACCTGCAGGGGGGGGGGGGCGCTG | |
| | AGGTCTGCCTCGTGAAGAAGGTGTTGCTGACTCATACCAGGCCTGAATCGCCCCATCATCC | |
| | AGCCAGAAAGTGAGGGAGCCACGGTTGATGAGAGCTTTGTTGTAGGTGGACCAGTTGGTGA | |
| | TTTTGAACTTTTGCTTTGCCACGGAACGGTCTGCGTTGTCGGGAAGATGCGTGATCTGATC | |
| | CTTCAACTCAGCAAAAGTTCGATTTATTCAACAAAGCCGCCGTCCCGTCAAGTCAGCGTAA | |
| | TGCTCTGCCAGTGTTACAACCAATTAACCAATTCTGATTAGAAAAACTCATCGAGCATCAA | |
| | ATGAAACTGCAATTTATTCATATCAGGATTATCAATACCATATTTTTGAAAAAGCCGTTTC | |
| | TGTAATGAAGGAGAAAACTCACCGAGGCAGTTCCATAGGATGGCAAGATCCTGGTATCGGT | |
| | CTGCGATTCCGACTCGTCCAACATCAATACAACCTATTAATTTCCCCTCGTCAAAAATAAG | |
| | GTTATCAAGTGAGAAATCACCATGAGTGACGACTGAATCCGGTGAGAATGGCAAAAGCTTA | |
| | TGCATTTCTTTCCAGACTTGTTCAACAGGCCAGCCATTACGCTCGTCATCAAAATCACTCG | |
| | CATCAACCAAACCGTTATTCATTCGTGATTGCGCCTGAGCGAGACGAAATACGCGATCGCT | |
| | GTTAAAAGGACAATTACAAACAGGAATCGAATGCAACCGGCGCAGGAACACTGCCAGCGCA | |
| | TCAACAATATTTTCACCTGAATCAGGATATTCTTCTAATACCTGGAATGCTGTTTTCCCGG | |
| | GGATCGCAGTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACATCATTGGCA | |
| | ACGCTACCTTTGCCATGTTTCAGAAACAACTCTGGCGCATCGGGCTTCCCATACAATCGAT | |
| | AGATTGTCGCACCTGATTGCCCGACATTATCGCGAGCCCATTTATACCCATATAAATCAGC | |
| | ATCCATGTTGGAATTTAATCGCGGCCTCGAGCAAGACGTTTCCCGTTGAATATGGCTCAT | |
| AP1_{H3} | TTGAGACACAACGTGGCTTTCCATCAAAAAAATATTGACAACATAAAAAACTTTGTGTTAT | 89 |
| | ACTTGTGGAATTGTGAGCGGATAACAATTCTATATCTGTTATTTTTTCATATACAAGATCT | |
| | atgaaaaaattattattcgcaattcctttagttgttcctttctattctcactccgctgaaa | |
| | ctgttgaaagttgtttagcaaaaccccatacagaaaattcatttactaacgtctggaaaga | |
| | cgacaaaactttagatcgttacgctaactatgagggctgtctgtggaatgctacaggcgtt | |
| | gtagtttgtactggtgacgaaactcagtgttacggtacatgggttcctattgggcttgcta | |
| | tccctgaaaatgagggtggtggctctgagggtggtggttctgagggtggcggttctgaggg | |
| | tggcggtactaaacctcctgagtacggtgatacacctattccgggctatacttatatcaac | |
| | cctctcgacggcacttatccgcctggtactgagcaaaaccccgctaatcctaatccttctc | |
| | ttgaggagtctcagcctcttaatactttcatgtttcagaataataggttccgaaataggca | |
| | gggggcattaactgtttatacgggcactgttactcaaggcactgaccccgttaaaacttat | |
| | taccagtacactcctgtatcatcaaaagccatgtatgacgcttactggaacggtaaattca | |
| | gagactgcgctttccattctggctttaatgaggatccattcgtttgtgaatatcaaggcca | |
| | atcgtctgacctgcctcaacctcctgtcaatgctggcggcggctctggtggtggttctggt | |
| | ggcggctctgagggtggtggctctgagggtggcggttctgagggtggcggctctgagggag | |
| | gcggttccggtggtggctctggttccggtgattttgattatgaaaagatggcaaacgctaa | |
| | taagggggctatgaccgaaaatgccgatgaaaacgcgctacagtctgacgctaaaggcaaa | |
| | cttgattctgtcgctactgattacggtgctgctatcgatggtttcattggtgacgtttccg | |
| | gccttgctaatggtaatggtgctactggtgattttgctggctctaattcccaaatggctca | |
| | agtcggtgacggtgataattcacctttaatgaataatttccgtcaatatttaccttccctc | |
| | cctcaatcggttgaatgtcgcccttttgtctttggcgctggtaaaccttacgagttcagta | |
| | tcgactgcgataagatcaacctgttccgcggtgtctttgcgtttcttttatatgttgccac | |
| | ctttatgtatgtattttctacgtttgctaacatactgcgtaataaggagtcttaaGTCGAC | |
| | CGGCTGCTAACAAAGCCCGCGGCCGCTGAAGATCGATCTCGACGAGTGAGAGAAGATTTTC | |
| | AGCCTGATACAGATTAAATCAGAACGCAGAAGCGGTCTGATAAAACAGAATTTGCCTGGCG | |
| | GCAGTAGCGCGGTGGTCCCACCTGACCCCATGCCGAACTCAGAAGTGAAACGCCGTAGCGC | |
| | CGATGGTAGTGTGGGGTCACCCCATGCGAGAGTAGGGAACTGCCAGGCATCAAATAAAACG | |
| | AAAGGCTCAGTCGAAAGACTGGGCCTTTCGTTTTATCTGTTGTTTGTCGGTGAACGCTCTC | |
| | CTGAGTAGGACAAATCCGCCGGGAGCGGATTTGAACGTTGCGAAGCAACGGCCCGGAGGGT | |
| | GGCGGGCAGGACGCCCGCCATAAACTGCCAGGCATCAAATTAAGCAGAAGGCCATCCTGAC | |
| | GGATGGCCTTTTTGCGTTTCTACAGAGCGTCAGACCCCTTAATAAGATGATCTTCTTGAGA | |
| | TCGTTTTGGTCTGCGCGTAATCTCTTGCTCTGAAAACGAAAAAACCGCCTTGCAGGGCGGT | |
| | TTTTCGAAGGTTCTCTGAGCTACCAACTCTTTGAACCGAGGTAACTGGCTTGGAGGAGCGC | |
| | AGTCACCAAAACTTGTCCTTTCAGTTTAGCCTTAACCGGCGCATGACTTCAAGACTAACTC | |
| | CTCTAAATCAATTACCAGTGGCTGCTGCCAGTGGTGCTTTTGCATGTCTTTCCGGGTTGGA | |
| | CTCAAGACGATAGTTACCGGATAAGGCGCAGCGGTCGGACTGAACGGGGGGTTCGTGCATA | |
| | CAGTCCAGCTTGGAGCGAACTGCCTACCCGGAACTGAGTGTCAGGCGTGGAATGAGACAAA | |
| | CGCGGCCATAACAGCGGAATGACACCGGTAAACCGAAAGGCAGGAACAGGAGAGCGCACGA | |
| | GGGAGCCGCCAGGGGGAAACGCCTGGTATCTTTATAGTCCTGTCGGGTTTCGCCACCACTG | |
| | ATTTGAGCGTCAGATTTCGTGATGCTTGTCAGGGGGGCGGAGCCTATGGAAAAACGGCTTT | |
| | GCCGCGGCCCTCTCGGATCTGTATGGTGCACTCTCAGTACAATCTGCTCTGATGCCGCATA | |
| | GTTAAGCCAGTATACACTCCGCTATCGCTACGTGACTGCCTCGACCTGCAGCAATTCCAAC | |
| | GCCATCAAAAATAATTCGCGTCTGGCCTTCCTGTAGCCAGCTTTCATCAACATTAAATGTG | |
| | AGCGAGTAACAACCCGTCGGATTCTCCGTGGGAACAAACGGCGGATTGACCGTAATGGGAT | |
| | AGGTCACGTTGGTGTAGATGGGCGCATCGTAACCGTGCATCTGCCAGTTTGAGGGGACGAC | |
| | GACAGTATCGGCCTCAGGAAGATCGCACTCCAGCCAGCTTTCCGGCACCGCTTCTGGTGCC | |
| | GGAAACCAGGCAAAGCGCCATTCGCCATTCAGGCTGCGCAACTGTTGGGAAGGGCGATCGG | |
| | TGCGGGCCTCTTCGCTATTACGCCAGCTGGCGAAAGGGGGATGTGCTGCAAGGCGATTAAG | |
| | TTGGGTAACGCCAGGGTTTTCCCAGTCACGACGTTGTAAAACGACGGCCAGTGAATCCGTA | |
| | ATCATGGTCATAGCTGTTTCCTGTGTGAAATTGTTATCCGCTCACAATTCCACACAACATA | |
| | CGAGCCGGAAGCATAAAGTGTAAAGCCTGGGGTGCCTAATGAGTGAGCTAACTTACATTAA | |
| | TTGCGTTGCGCTCACTGCCCGCTTTCCAGTCGGGAAACCTGTCGTGCCAGCTGCATTAATG | |
| | AATCGGCCAACGCGCGGGGAGAGGCGGTTTGCGTATTGGGCGCCAGGGTGGTTTTTCTTTT | |
| | CACCAGTGAGACGGGCAACAGCTGATTGCCCTTCACCGCCTGGCCCTGAGAGAGTTGCAGC | |
| | AAGCGGTCCACGCTGGTTTGCCCCAGCAGGCGAAAATCCTGTTTGATGGTGGTTAACGGCG | |
| | GGATATAACATGAGCTGTCTTCGGTATCGTCGTATCCCACTACCGAGATATCCGCACCAAC | |
| | GCGCAGCCCGGACTCGGTAATGGCGCGCATTGCGCCCAGCGCCATCTGATCGTTGGCAACC | |
| | AGCATCGCAGTGGGAACGATGCCCTCATTCAGCATTTGCATGGTTTGTTGAAAACCGGACA | |
| | TGGCACTCCAGTCGCCTTCCCGTTCCGCTATCGGCTGAATTTGATTGCGAGTGAGATATTT | |
| | ATGCCAGCCAGCCAGACGCAGACGCGCCGAGACAGAACTTAATGGGCCCGCTAACAGCGCG | |
| | ATTTGCTGGTGACCCAATGCGACCAGATGCTCCACGCCCAGTCGCGTACCGTCTTCATGGG | |
| | AGAAAATAATACTGTTGATGGGTGTCTGGTCAGAGACATCAAGAAATAACGCCGGAACATT | |
| | AGTGCAGGCAGCTTCCACAGCAATGGCATCCTGGTCATCCAGCGGATAGTTAATGATCAGC | |
| | CCACTGACGCGTTGCGCGAGAAGATTGTGCACCGCCGCTTTACAGGCTTCGACGCCGCTTC | |
| | GTTCTACCATCGACACCACCACGCTGGCACCCAGTTGATCGGCGCGAGATTTAATCGCCGC | |
| | GACAATTTGCGACGGCGCGTGCAGGGCCAGACTGGAGGTGGCAACGCCAATCAGCAACGAC | |
| | TGTTTGCCCGCCAGTTGTTGTGCCACGCGGTTGGGAATGTAATTCAGCTCCGCCATCGCCG | |
| | CTTCCACTTTTTCCCGCGTTTTCGCAGAAACGTGGCTGGCCTGGTTCACCACGCGGGAAAC | |
| | GGTCTGATAAGAGACACCGGCATACTCTGCGACATCGTATAACGTTACTGGTTTCACATTC | |
| | ACCACCCTGAATTGACTCTCTTCCGGGCGCTATCATGCCATACCGCGAAAGGTTTTGCACC | |
| | ATTCCATGGTGTCGGAATTGCTGCAGGTCGAGGGGGTCATGGCTGCGCCCCGACACCCGCC | |
| | AACACCCGCTGACGCGCCCTGACGGGCTTGTCTGCTCCCGGCATCCGCTTACAGACAAGCT | |
| | GTGACCGTCTCCGGGAGCTGCATGTGTCAGAGGTTTTCACCGTCATCACCGAAACGCGCGA | |
| | GGCAGGATGGCGCCCAACAGTCCCCCGGCCACGGGGCCTGCCACCATACCCACGCCGAAAC | |
| | AAGCGCTCATGAGCCCGAAGTGGCGAGCCCGATCTTCCCCATCGGTGATGTCGGCGATATA | |
| | GGCGCCAGCAACCGCACCTGTGGCGCCGGTGATGCCGGCCACGATGCGTCCGGCGTAGAGG | |
| | ATCCGTCGACCTGCAGGGGGGGGGGGGCGCTGAGGTCTGCCTCGTGAAGAAGGTGTTGCTG | |
| | ACTCATACCAGGCCTGAATCGCCCCATCATCCAGCCAGAAAGTGAGGGAGCCACGGTTGAT | |
| | GAGAGCTTTGTTGTAGGTGGACCAGTTGGTGATTTTGAACTTTTGCTTTGCCACGGAACGG | |
| | TCTGCGTTGTCGGGAAGATGCGTGATCTGATCCTTCAACTCAGCAAAAGTTCGATTTATTC | |
| | AACAAAGCCGCCGTCCCGTCAAGTCAGCGTAATGCTCTGCCAGTGTTACAACCAATTAACC | |
| | AATTCTGATTAGAAAAACTCATCGAGCATCAAATGAAACTGCAATTTATTCATATCAGGAT | |
| | TATCAATACCATATTTTTGAAAAAGCCGTTTCTGTAATGAAGGAGAAAACTCACCGAGGCA | |
| | GTTCCATAGGATGGCAAGATCCTGGTATCGGTCTGCGATTCCGACTCGTCCAACATCAATA | |
| | CAACCTATTAATTTCCCCTCGTCAAAAATAAGGTTATCAAGTGAGAAATCACCATGAGTGA | |
| | CGACTGAATCCGGTGAGAATGGCAAAAGCTTATGCATTTCTTTCCAGACTTGTTCAACAGG | |
| | CCAGCCATTACGCTCGTCATCAAAATCACTCGCATCAACCAAACCGTTATTCATTCGTGAT | |
| | TGCGCCTGAGCGAGACGAAATACGCGATCGCTGTTAAAAGGACAATTACAAACAGGAATCG | |
| | AATGCAACCGGCGCAGGAACACTGCCAGCGCATCAACAATATTTTCACCTGAATCAGGATA | |
| | TTCTTCTAATACCTGGAATGCTGTTTTCCCGGGGATCGCAGTGGTGAGTAACCATGCATCA | |
| | TCAGGAGTACGGATAAAATGCTTGATGGTCGGAAGAGGCATAAATTCCGTCAGCCAGTTTA | |
| | GTCTGACCATCTCATCTGTAACATCATTGGCAACGCTACCTTTGCCATGTTTCAGAAACAA | |
| | CTCTGGCGCATCGGGCTTCCCATACAATCGATAGATTGTCGCACCTGATTGCCCGACATTA | |
| | TCGCGAGCCCATTTATACCCATATAAATCAGCATCCATGTTGGAATTTAATCGCGGCCTCG | |
| | AGCAAGACGTTTCCCGTTGAATATGGCTCATAACACCCCTTGTATTACTGTTTATGTAAGC | |
| | AGACAGTTTTATTGTTCATGATGATATATTTTTATCTTGTGCAATGTAACATCAGAGATT | |
| AP2 | tcagatccttccgtatttagccagtatgttctctagtgtggttcgttgtttttgcgtgagc | 90 |
| | catgagaacgaaccattgagatcatgcttactttgcatgtcactcaaaaattttgcctcaa | |
| | aactggtgagctgaatttttgcagttaaagcatcgtgtagtgtttttcttagtccgttacg | |
| | taggtaggaatctgatgtaatggttgttggtattttgtcaccattcatttttatctggttg | |
| | ttctcaagttcggttacgagatccatttgtctatctagttcaacttggaaaatcaacgtat | |
| | cagtcgggcggcctcgcttatcaaccaccaatttcatattgctgtaagtgtttaaatcttt | |
| | acttattggtttcaaaacccattggttaagccttttaaactcatggtagttattttcaagc | |
| | attaacatgaacttaaattcatcaaggctaatctctatatttgccttgtgagttttctttt | |
| | gtgttagttcttttaataaccactcataaatcctcatagagtatttgttttcaaaagactt | |
| | aacatgttccagattatattttatgaatttttttaactggaaaagataaggcaatatctct | |
| | tcactaaaaactaattctaatttttcgcttgagaacttggcatagtttgtccactggaaaa | |
| | tctcaaagcctttaaccaaaggattcctgatttccacagttctcgtcatcagctctctggt | |
| | tgctttagctaatacaccataagcattttccctactgatgttcatcatctgagcgtattgg | |
| | ttataagtgaacgataccgtccgttctttccttgtagggttttcaatcgtggggttgagta | |
| | gtgccacacagcataaaattagcttggtttcatgctccgttaagtcatagcgactaatcgc | |
| | tagttcatttgctttgaaaacaactaattcagacatacatctcaattggtctaggtgattt | |
| | taatcactataccaattgagatgggctagtcaatgataattactagtccttttcctttgag | |
| | ttgtgggtatctgtaaattctgctagacctttgctggaaaacttgtaaattctgctagacc | |
| | ctctgtaaattccgctagacctttgtgtgttttttttgtttatattcaagtggttataatt | |
| | tatagaataaagaaagaataaaaaaagataaaaagaatagatcccagccctgtgtataact | |
| | cactactttagtcagttccgcagtattacaaaaggatgtcgcaaacgctgtttgctcctct | |
| | acaaaacagaccttaaaaccctaaaggcttaagtagcaccctcgcaagctcgggcaaatcg | |
| | ctgaatattccttttgtctccgaccatcaggcacctgagtcgctgtctttttcgtgacatt | |
| | cagttcgctgcgctcacggctctggcagtgaatgggggtaaatggcactacaggcgccttt | |
| | tatggattcatgcaaggaaactacccataatacaagaaaagcccgtcacgggcttctcagg | |
| | gcgttttatggcgggtctgctatgtggtgctatctgactttttgctgttcagcagttcctg | |
| | ccctctgattttccagtctgaccacttcggattatcccgtgacaggtcattcagactggct | |
| | aatgcacccagtaaggcagcggtatcatcaacaggcttacccgtcttactgtcaagaggac | |
| | atccggtggctgttttggcggatgagagaagattttcagcctgatacagattaaatcagaa | |
| | cgcagaagcggtctgataaaacagaatttgcctggcggcagtagcgcggtggtcccacctg | |
| | accccatgccgaactcagaagtgaaacgccgtagcgccgatggtagtgtggggtctcccca | |
| | tgcgagagtagggaactgccaggcatcaaataaaacgaaaggctcagtcgaaagactgggc | |
| | ctttcgttttatctgttgtttgtcggtgaacgctctcctgagtaggacaaatccgccggga | |
| | gcggatttgaacgttgcgaagcaacggcccggagggtggcgggcaggacgcccgccataaa | |
| | ctgccaggcatcaaattaagcagaaggccatcctgacggatggcctttttgcgtttctaca | |
| | aactcgctgaggattctagagCACAGCTAACACCACGTCGTCCCTATCTGCTGCCCTAGGT | |
| | CTATGAGTGGTTGCTGGATAACTTTACGGGCATGCATAAGGCTCGTATGATATATTCAGGG | |
| | AGACCACAACGGTTTCCCTCTACAAATAATTTTGTTTAACTTTATATCTGTTATTTTTTCA | |
| | ACCACAGATCTatgaaaaaattattattcgcaattcctttagttgttcctttctattctca | |
| | ctccgctgaaactgttgaaagttgtttagcaaaaccccatacagaaaattcatttactaac | |
| | gtctggaaagacgacaaaactttagatcgttacgctaactatgagggctgtctgtggaatg | |
| | ctacaggcgttgtagtttgtactggtgacgaaactcagtgttacggtacatgggttcctat | |
| | tgggcttgctatccctgaaaatgagggtggtggctctgagggtggtggttctgagggtggc | |
| | ggttctgagggtggcggtactaaacctcctgagtacggtgatacacctattccgggctata | |
| | cttatatcaaccctctcgacggcacttatccgcctggtactgagcaaaaccccgctaatcc | |
| | taatccttctcttgaggagtctcagcctcttaatactttcatgtttcagaataataggttc | |
| | cgaaataggcagggggcattaactgtttatacgggcactgttactcaaggcactgaccccg | |
| | ttaaaacttattaccagtacactcctgtatcatcaaaagccatgtatgacgcttactggaa | |
| | cggtaaattcagagactgcgctttccattctggctttaatgaggatccattcgtttgtgaa | |
| | tatcaaggccaatcgtctgacctgcctcaacctcctgtcaatgctggcggcggctctggtg | |
| | gtggttctggtggcggctctgagggtggtggctctgagggtggcggttctgagggtggcgg | |
| | ctctgagggaggcggttccggtggtggctctggttccggtgattttgattatgaaaagatg | |
| | gcaaacgctaataagggggctatgaccgaaaatgccgatgaaaacgcgctacagtctgacg | |
| | ctaaaggcaaacttgattctgtcgctactgattacggtgctgctatcgatggtttcattgg | |
| | tgacgtttccggccttgctaatggtaatggtgctactggtgattttgctggctctaattcc | |
| | caaatggctcaagtcggtgacggtgataattcacctttaatgaataatttccgtcaatatt | |
| | taccttccctccctcaatcggttgaatgtcgcccttttgtctttggcgctggtaaacctta | |
| | cgagttcagtatcgactgcgataagatcaacctgttccgcggtgtctttgcgtttctttta | |
| | tatgttgccacctttatgtatgtattttctacgtttgctaacatactgcgtaataaggagt | |
| | cttaaacttaattaacggcactcctcagccaagtcaaaagcctccgaccggaggcttttga | |
| | ctacatgcccatggcgtttagaaaaactcatcgagcatcaaatgaaactgcaatttattca | |
| | tatcaggattatcaataccatatttttgaaaaagccgtttctgtaatgaaggagaaaactc | |
| | accgaggcagttccataggatggcaagatcctggtatcggtctgcgattccgactcgtcca | |
| | acatcaatacaacctattaatttcccctcgtcaaaaataaggttatcaagtgagaaatcac | |
| | catgagtgacgactgaatccggtgagaatggcaaaagcttatgcatttctttccagacttg | |
| | ttcaacaggccagccattacgctcgtcatcaaaatcactcgcatcaaccaaaccgttattc | |
| | attcgtgattgcgcctgagcgagacgaaatacgcgatcgctgttaaaaggacaattacaaa | |
| | caggaatcgaatgcaaccggcgcaggaacactgccagcgcatcaacaatattttcacctga | |
| | atcaggatattcttctaatacctggaatgctgttttcccggggatcgcagtggtgagtaac | |
| | catgcatcatcaggagtacggataaaatgcttgatggtcggaagaggcataaattccgtca | |
| | gccagtttagtctgaccatctcatctgtaacatcattggcaacgctacctttgccatgttt | |
| | cagaaacaactctggcgcatcgggcttcccatacaatcgatagattgtcgcacctgattgc | |
| | ccgacattatcgcgagcccatttatacccatataaatcagcatccatgttggaatttaatc | |
| | gcggcctcgagcaagacgtttcccgttgaatatggctcataacaccccttgtattactgtt | |
| | tatgtaagcagacagttttattgttcatgatgatatatttttatcttgtgcaatgtaacat | |
| | cagagattttgaaggccaaataggccgt | |
| AP2_{H3} | tcagatccttccgtatttagccagtatgttctctagtgtggttcgttgtttttgcgtgagc | 91 |
| | catgagaacgaaccattgagatcatgcttactttgcatgtcactcaaaaattttgcctcaa | |
| | aactggtgagctgaatttttgcagttaaagcatcgtgtagtgtttttcttagtccgttacg | |
| | taggtaggaatctgatgtaatggttgttggtattttgtcaccattcatttttatctggttg | |
| | ttctcaagttcggttacgagatccatttgtctatctagttcaacttggaaaatcaacgtat | |
| | cagtcgggcggcctcgcttatcaaccaccaatttcatattgctgtaagtgtttaaatcttt | |
| | acttattggtttcaaaacccattggttaagccttttaaactcatggtagttattttcaagc | |
| | attaacatgaacttaaattcatcaaggctaatctctatatttgccttgtgagttttctttt | |
| | gtgttagttcttttaataaccactcataaatcctcatagagtatttgttttcaaaagactt | |
| | aacatgttccagattatattttatgaatttttttaactggaaaagataaggcaatatctct | |
| | tcactaaaaactaattctaatttttcgcttgagaacttggcatagtttgtccactggaaaa | |
| | tctcaaagcctttaaccaaaggattcctgatttccacagttctcgtcatcagctctctggt | |
| | tgctttagctaatacaccataagcattttccctactgatgttcatcatctgagcgtattgg | |
| | ttataagtgaacgataccgtccgttctttccttgtagggttttcaatcgtggggttgagta | |
| | gtgccacacagcataaaattagcttggtttcatgctccgttaagtcatagcgactaatcgc | |
| | tagttcatttgctttgaaaacaactaattcagacatacatctcaattggtctaggtgattt | |
| | taatcactataccaattgagatgggctagtcaatgataattactagtccttttcctttgag | |
| | ttgtgggtatctgtaaattctgctagacctttgctggaaaacttgtaaattctgctagacc | |
| | ctctgtaaattccgctagacctttgtgtgttttttttgtttatattcaagtggttataatt | |
| | tatagaataaagaaagaataaaaaaagataaaaagaatagatcccagccctgtgtataact | |
| | cactactttagtcagttccgcagtattacaaaaggatgtcgcaaacgctgtttgctcctct | |
| | acaaaacagaccttaaaaccctaaaggcttaagtagcaccctcgcaagctcgggcaaatcg | |
| | ctgaatattccttttgtctccgaccatcaggcacctgagtcgctgtctttttcgtgacatt | |
| | cagttcgctgcgctcacggctctggcagtgaatgggggtaaatggcactacaggcgccttt | |
| | tatggattcatgcaaggaaactacccataatacaagaaaagcccgtcacgggcttctcagg | |
| | gcgttttatggcgggtctgctatgtggtgctatctgactttttgctgttcagcagttcctg | |
| | ccctctgattttccagtctgaccacttcggattatcccgtgacaggtcattcagactggct | |
| | aatgcacccagtaaggcagcggtatcatcaacaggcttacccgtcttactgtcaagaggac | |
| | atccggtggctgttttggcggatgagagaagattttcagcctgatacagattaaatcagaa | |
| | cgcagaagcggtctgataaaacagaatttgcctggcggcagtagcgcggtggtcccacctg | |
| | accccatgccgaactcagaagtgaaacgccgtagcgccgatggtagtgtggggtctcccca | |
| | tgcgagagtagggaactgccaggcatcaaataaaacgaaaggctcagtcgaaagactgggc | |
| | ctttcgttttatctgttgtttgtcggtgaacgctctcctgagtaggacaaatccgccggga | |
| | gcggatttgaacgttgcgaagcaacggcccggagggtggcgggcaggacgcccgccataaa | |
| | ctgccaggcatcaaattaagcagaaggccatcctgacggatggcctttttgcgtttctaca | |
| | aactcgctgaggattctagagCACAGCTAACACCACGTCGTCCCTATCTGCTGCCCTAGGT | |
| | CTATGAGTGGTTGCTGGATAACTTTACGGGCATGCATAAGGCTCGTATGATATATTCAGGG | |
| | AGACCACAACGGTTTCCCTCTACAAATAATTTTGTTTAACTTTATATCTGTTATTTTTTCA | |
| | TATACAAGATCTatgaaaaaattattattcgcaattcctttagttgttcctttctattctc | |
| | actccgctgaaactgttgaaagttgtttagcaaaaccccatacagaaaattcatttactaa | |
| | cgtctggaaagacgacaaaactttagatcgttacgctaactatgagggctgtctgtggaat | |
| | gctacaggcgttgtagtttgtactggtgacgaaactcagtgttacggtacatgggttccta | |
| | ttgggcttgctatccctgaaaatgagggtggtggctctgagggtggtggttctgagggtgg | |
| | cggttctgagggtggcggtactaaacctcctgagtacggtgatacacctattccgggctat | |
| | acttatatcaaccctctcgacggcacttatccgcctggtactgagcaaaaccccgctaatc | |
| | ctaatccttctcttgaggagtctcagcctcttaatactttcatgtttcagaataataggtt | |
| | ccgaaataggcagggggcattaactgtttatacgggcactgttactcaaggcactgacccc | |
| | gttaaaacttattaccagtacactcctgtatcatcaaaagccatgtatgacgcttactgga | |
| | acggtaaattcagagactgcgctttccattctggctttaatgaggatccattcgtttgtga | |
| | atatcaaggccaatcgtctgacctgcctcaacctcctgtcaatgctggcggcggctctggt | |
| | ggtggttctggtggcggctctgagggtggtggctctgagggtggcggttctgagggtggcg | |
| | gctctgagggaggcggttccggtggtggctctggttccggtgattttgattatgaaaagat | |
| | ggcaaacgctaataagggggctatgaccgaaaatgccgatgaaaacgcgctacagtctgac | |
| | gctaaaggcaaacttgattctgtcgctactgattacggtgctgctatcgatggtttcattg | |
| | gtgacgtttccggccttgctaatggtaatggtgctactggtgattttgctggctctaattc | |
| | ccaaatggctcaagtcggtgacggtgataattcacctttaatgaataatttccgtcaatat | |
| | ttaccttccctccctcaatcggttgaatgtcgcccttttgtctttggcgctggtaaacctt | |
| | acgagttcagtatcgactgcgataagatcaacctgttccgcggtgtctttgcgtttctttt | |
| | atatgttgccacctttatgtatgtattttctacgtttgctaacatactgcgtaataaggag | |
| | tcttaaacttaattaacggcactcctcagccaagtcaaaagcctccgaccggaggcttttg | |
| | actacatgcccatggcgtttagaaaaactcatcgagcatcaaatgaaactgcaatttattc | |
| | atatcaggattatcaataccatatttttgaaaaagccgtttctgtaatgaaggagaaaact | |
| | caccgaggcagttccataggatggcaagatcctggtatcggtctgcgattccgactcgtcc | |
| | aacatcaatacaacctattaatttcccctcgtcaaaaataaggttatcaagtgagaaatca | |
| | ccatgagtgacgactgaatccggtgagaatggcaaaagcttatgcatttctttccagactt | |
| | gttcaacaggccagccattacgctcgtcatcaaaatcactcgcatcaaccaaaccgttatt | |
| | cattcgtgattgcgcctgagcgagacgaaatacgcgatcgctgttaaaaggacaattacaa | |
| | acaggaatcgaatgcaaccggcgcaggaacactgccagcgcatcaacaatattttcacctg | |
| | aatcaggatattcttctaatacctggaatgctgttttcccggggatcgcagtggtgagtaa | |
| | ccatgcatcatcaggagtacggataaaatgcttgatggtcggaagaggcataaattccgtc | |
| | agccagtttagtctgaccatctcatctgtaacatcattggcaacgctacctttgccatgtt | |
| | tcagaaacaactctggcgcatcgggcttcccatacaatcgatagattgtcgcacctgattg | |
| | cccgacattatcgcgagcccatttatacccatataaatcagcatccatgttggaatttaat | |
| | cgcggcctcgagcaagacgtttcccgttgaatatggctcataacaccccttgtattactgt | |
| | ttatgtaagcagacagttttattgttcatgatgatatatttttatcttgtgcaatgtaaca | |
| | tcagagattttgaaggccaaataggccgt | |
| AP3_{H3} | tcagatccttccgtatttagccagtatgttctctagtgtggttcgttgtttttgcgtgagc | 92 |
| | catgagaacgaaccattgagatcatgcttactttgcatgtcactcaaaaattttgcctcaa | |
| | aactggtgagctgaatttttgcagttaaagcatcgtgtagtgtttttcttagtccgttacg | |
| | taggtaggaatctgatgtaatggttgttggtattttgtcaccattcatttttatctggttg | |
| | ttctcaagttcggttacgagatccatttgtctatctagttcaacttggaaaatcaacgtat | |
| | cagtcgggcggcctcgcttatcaaccaccaatttcatattgctgtaagtgtttaaatcttt | |
| | acttattggtttcaaaacccattggttaagccttttaaactcatggtagttattttcaagc | |
| | attaacatgaacttaaattcatcaaggctaatctctatatttgccttgtgagttttctttt | |
| | gtgttagttcttttaataaccactcataaatcctcatagagtatttgttttcaaaagactt | |
| | aacatgttccagattatattttatgaatttttttaactggaaaagataaggcaatatctct | |
| | tcactaaaaactaattctaatttttcgcttgagaacttggcatagtttgtccactggaaaa | |
| | tctcaaagcctttaaccaaaggattcctgatttccacagttctcgtcatcagctctctggt | |
| | tgctttagctaatacaccataagcattttccctactgatgttcatcatctgagcgtattgg | |
| | ttataagtgaacgataccgtccgttctttccttgtagggttttcaatcgtggggttgagta | |
| | gtgccacacagcataaaattagcttggtttcatgctccgttaagtcatagcgactaatcgc | |
| | tagttcatttgctttgaaaacaactaattcagacatacatctcaattggtctaggtgattt | |
| | taatcactataccaattgagatgggctagtcaatgataattactagtccttttcctttgag | |
| | ttgtgggtatctgtaaattctgctagacctttgctggaaaacttgtaaattctgctagacc | |
| | ctctgtaaattccgctagacctttgtgtgttttttttgtttatattcaagtggttataatt | |
| | tatagaataaagaaagaataaaaaaagataaaaagaatagatcccagccctgtgtataact | |
| | cactactttagtcagttccgcagtattacaaaaggatgtcgcaaacgctgtttgctcctct | |
| | acaaaacagaccttaaaaccctaaaggcttaagtagcaccctcgcaagctcgggcaaatcg | |
| | ctgaatattccttttgtctccgaccatcaggcacctgagtcgctgtctttttcgtgacatt | |
| | cagttcgctgcgctcacggctctggcagtgaatgggggtaaatggcactacaggcgccttt | |
| | tatggattcatgcaaggaaactacccataatacaagaaaagcccgtcacgggcttctcagg | |
| | gcgttttatggcgggtctgctatgtggtgctatctgactttttgctgttcagcagttcctg | |
| | ccctctgattttccagtctgaccacttcggattatcccgtgacaggtcattcagactggct | |
| | aatgcacccagtaaggcagcggtatcatcaacaggcttacccgtcttactgtcaagaggac | |
| | atccggtttgttcagaacgctcggtcttgcacaccgggcgttttttctttgtgagtccata | |
| | gtacacttgaataaataaaaacagccgttgccagaaagaggcacggctgtttttattttag | |
| | acttagggaccctcacagctaacaccacgtcgtccctatctgctgccctaggtctatgagt | |
| | ggttgctgGATAACTTTACGGGCATGCATAAGGCTCGTAATATATATTCagggagaccaca | |
| | acggtttccctctacaaataattttgtttaactttatatctgttattttttcatatacaag | |
| | atctatgaaaaaattattattcgcaattcctttagttgttcctttctattctcactccgct | |
| | gaaactgttgaaagttgtttagcaaaaccccatacagaaaattcatttactaacgtctgga | |
| | aagacgacaaaactttagatcgttacgctaactatgagggctgtctgtggaatgctacagg | |
| | cgttgtagtttgtactggtgacgaaactcagtgttacggtacatgggttcctattgggctt | |
| | gctatccctgaaaatgagggtggtggctctgagggtggtggttctgagggtggcggttctg | |
| | agggtggcggtactaaacctcctgagtacggtgatacacctattccgggctatacttatat | |
| | caaccctctcgacggcacttatccgcctggtactgagcaaaaccccgctaatcctaatcct | |
| | tctcttgaggagtctcagcctcttaatactttcatgtttcagaataataggttccgaaata | |
| | ggcagggggcattaactgtttatacgggcactgttactcaaggcactgaccccgttaaaac | |
| | ttattaccagtacactcctgtatcatcaaaagccatgtatgacgcttactggaacggtaaa | |
| | ttcagagactgcgctttccattctggctttaatgaggatccattcgtttgtgaatatcaag | |
| | gccaatcgtctgacctgcctcaacctcctgtcaatgctggcggcggctctggtggtggttc | |
| | tggtggcggctctgagggtggtggctctgagggtggcggttctgagggtggcggctctgag | |
| | ggaggcggttccggtggtggctctggttccggtgattttgattatgaaaagatggcaaacg | |
| | ctaataagggggctatgaccgaaaatgccgatgaaaacgcgctacagtctgacgctaaagg | |
| | caaacttgattctgtcgctactgattacggtgctgctatcgatggtttcattggtgacgtt | |
| | tccggccttgctaatggtaatggtgctactggtgattttgctggctctaattcccaaatgg | |
| | ctcaagtcggtgacggtgataattcacctttaatgaataatttccgtcaatatttaccttc | |
| | cctccctcaatcggttgaatgtcgcccttttgtctttggcgctggtaaaccttacgagttc | |
| | agtatcgactgcgataagatcaacctgttccgcggtgtctttgcgtttcttttatatgttg | |
| | ccacctttatgtatgtattttctacgtttgctaacatactgcgtaataaggagtcttaaac | |
| | ttaattaacggcactcctcagccaagtcaaaagcctccgaccggaggcttttgactacatg | |
| | cccatggcgtttagaaaaactcatcgagcatcaaatgaaactgcaatttattcatatcagg | |
| | attatcaataccatatttttgaaaaagccgtttctgtaatgaaggagaaaactcaccgagg | |
| | cagttccataggatggcaagatcctggtatcggtctgcgattccgactcgtccaacatcaa | |
| | tacaacctattaatttcccctcgtcaaaaataaggttatcaagtgagaaatcaccatgagt | |
| | gacgactgaatccggtgagaatggcaaaagcttatgcatttctttccagacttgttcaaca | |
| | ggccagccattacgctcgtcatcaaaatcactcgcatcaaccaaaccgttattcattcgtg | |
| | attgcgcctgagcgagacgaaatacgcgatcgctgttaaaaggacaattacaaacaggaat | |
| | cgaatgcaaccggcgcaggaacactgccagcgcatcaacaatattttcacctgaatcagga | |
| | tattcttctaatacctggaatgctgttttcccggggatcgcagtggtgagtaaccatgcat | |
| | catcaggagtacggataaaatgcttgatggtcggaagaggcataaattccgtcagccagtt | |
| | tagtctgaccatctcatctgtaacatcattggcaacgctacctttgccatgtttcagaaac | |
| | aactctggcgcatcgggcttcccatacaatcgatagattgtcgcacctgattgcccgacat | |
| | tatcgcgagcccatttatacccatataaatcagcatccatgttggaatttaatcgcggcct | |
| | cgagcaagacgtttcccgttgaatatggctcataacaccccttgtattactgtttatgtaa | |
| | gcagacagttttattgttcatgatgatatatttttatcttgtgcaatgtaacatcagagat | |
| | tttgaaggccaaataggccgt | |
| intein- | tcagatccttccgtatttagccagtatgttctctagtgtggttcgttgtttttgcgtgagc | 93 |
| proBAP3H3 | catgagaacgaaccattgagatcatgcttactttgcatgtcactcaaaaattttgcctcaa | |
| | aactggtgagctgaatttttgcagttaaagcatcgtgtagtgtttttcttagtccgttacg | |
| | taggtaggaatctgatgtaatggttgttggtattttgtcaccattcatttttatctggttg | |
| | ttctcaagttcggttacgagatccatttgtctatctagttcaacttggaaaatcaacgtat | |
| | cagtcgggggcctcgcttatcaaccaccaatttcatattgctgtaagtgtttaaatcttt | |
| | acttattggtttcaaaacccattggttaagccttttaaactcatggtagttattttcaagc | |
| | attaacatgaacttaaattcatcaaggctaatctctatatttgccttgtgagttttctttt | |
| | gtgttagttcttttaataaccactcataaatcctcatagagtatttgttttcaaaagactt | |
| | aacatgttccagattatattttatgaatttttttaactggaaaagataaggcaatatctct | |
| | tcactaaaaactaattctaatttttcgcttgagaacttggcatagtttgtccactggaaaa | |
| | tctcaaagcctttaaccaaaggattcctgatttccacagttctcgtcatcagctctctggt | |
| | tgctttagctaatacaccataagcattttccctactgatgttcatcatctgagcgtattgg | |
| | ttataagtgaacgataccgtccgttctttccttgtagggttttcaatcgtggggttgagta | |
| | gtgccacacagcataaaattagcttggtttcatgctccgttaagtcatagcgactaatcgc | |
| | tagttcatttgctttgaaaacaactaattcagacatacatctcaattggtctaggtgattt | |
| | taatcactataccaattgagatgggctagtcaatgataattactagtccttttcctttgag | |
| | ttgtgggtatctgtaaattctgctagacctttgctggaaaacttgtaaattctgctagacc | |
| | ctctgtaaattccgctagacctttgtgtgttttttttgtttatattcaagtggttataatt | |
| | tatagaataaagaaagaataaaaaaagataaaaagaatagatcccagccctgtgtataact | |
| | cactactttagtcagttccgcagtattacaaaaggatgtcgcaaacgctgtttgctcctct | |
| | acaaaacagaccttaaaaccctaaaggcttaagtagcaccctcgcaagctcgggcaaatcg | |
| | ctgaatattccttttgtctccgaccatcaggcacctgagtcgctgtctttttcgtgacatt | |
| | cagttcgctgcgctcacggctctggcagtgaatgggggtaaatggcactacaggcgccttt | |
| | tatggattcatgcaaggaaactacccataatacaagaaaagcccgtcacgggcttctcagg | |
| | gcgttttatggcgggtctgctatgtggtgctatctgactttttgctgttcagcagttcctg | |
| | ccctctgattttccagtctgaccacttcggattatcccgtgacaggtcattcagactggct | |
| | aatgcacccagtaaggcagcggtatcatcaacaggcttacccgtcttactgtcaagaggac | |
| | atccggtttgttcagaacgctcggtcttgcacaccgggcgttttttctttgtgagtccata | |
| | gtacacttgaataaataaaaacagccgttgccagaaagaggcacggctgtttttattttag | |
| | acttagggaccctcacagctaacaccacgtcgtccctatctgctgccctaggtctatgagt | |
| | ggttgctgGATAACTTTACGGGCATGCATAAGGCTCGTAATATATATTCagggagaccaca | |
| | acggtttccctctacaaataattttgtttaactttatatctgttattttttcatatacaag | |
| | atctatgaaaaaattattattcgcaattcctttatgtctcagctacgaaaccgaaatcttg | |
| | accgtcgaatatggtctgctgccaatcggcaagattgttgaaaaacgtattgaatgtacgg | |
| | tctactcagtggataacaacggcaatatctacacccagccggtggcccagtggcatgaccg | |
| | tggtgaacaggaagtgttcgaatattgtctggaagacggatctttaatccgtgccacaaag | |
| | gatcacaaatttatgactgtagatggtcagatgctcccaatcgacgaaatttttgaacgcg | |
| | aattagacctgatgcgcgtggataatctcccgaatggtggtagcggtggttctatcaaaat | |
| | tgccacgcgtaaatatttaggcaaacagaatgtttatgatatcggtgtcgagcgcgatcat | |
| | aatttcgcgctgaaaaacggctttatcgccagcaattgttttaatgttgttcctttctatt | |
| | ctcactccgctgaaactgttgaaagttgtttagcaaaaccccatacagaaaattcatttac | |
| | taacgtctggaaagacgacaaaactttagatcgttacgctaactatgagggctgtctgtgg | |
| | aatgctacaggcgttgtagtttgtactggtgacgaaactcagtgttacggtacatgggttc | |
| | ctattgggcttgctatccctgaaaatgagggtggtggctctgagggtggcggttctgaggg | |
| | tggcggttctgagggtggcggtactaaacctcctgagtacggtgatacacctattccgggc | |
| | tatacttatatcaaccctctcgacggcacttatccgcctggtactgagcaaaaccccgcta | |
| | atcctaatccttctcttgaggagtctcagcctcttaatactttcatgtttcagaataatag | |
| | gttccgaaataggcagggggcattaactgtttatacgggcactgttactcaaggcactgac | |
| | cccgttaaaacttattaccagtacactcctgtatcatcaaaagccatgtatgacgcttact | |
| | ggaacggtaaattcagagactgcgctttccattctggctttaatgaggatccattcgtttg | |
| | tgaatatcaaggccaatcgtctgacctgcctcaacctcctgtcaatgctggcggcggctct | |
| | ggtggtggttctggtggcggctctgagggtggtggctctgagggtggcggttctgagggtg | |
| | gcggctctgagggaggcggttccggtggtggctctggttccggtgattttgattatgaaaa | |
| | gatggcaaacgctaataagggggctatgaccgaaaatgccgatgaaaacgcgctacagtct | |
| | gacgctaaaggcaaacttgattctgtcgctactgattacggtgctgctatcgatggtttca | |
| | ttggtgacgtttccggccttgctaatggtaatggtgctactggtgattttgctggctctaa | |
| | ttcccaaatggctcaagtcggtgacggtgataattcacctttaatgaataatttccgtcaa | |
| | tatttaccttccctccctcaatcggttgaatgtcgcccttttgtctttggcgctggtaaac | |
| | cttacgagttcagtatcgactgcgataagatcaacctgttccgcggtgtctttgcgtttct | |
| | tttatatgttgccacctttatgtatgtattttctacgtttgctaacatactgcgtaataag | |
| | gagtcttaaacttaattaacggcactcctcagccaagtcaaaagcctccgaccggaggctt | |
| | ttgactacatgcccatggcgtttagaaaaactcatcgagcatcaaatgaaactgcaattta | |
| | ttcatatcaggattatcaataccatatttttgaaaaagccgtttctgtaatgaaggagaaa | |
| | actcaccgaggcagttccataggatggcaagatcctggtatcggtctgcgattccgactcg | |
| | tccaacatcaatacaacctattaatttcccctcgtcaaaaataaggttatcaagtgagaaa | |
| | tcaccatgagtgacgactgaatccggtgagaatggcaaaagcttatgcatttctttccaga | |
| | cttgttcaacaggccagccattacgctcgtcatcaaaatcactcgcatcaaccaaaccgtt | |
| | attcattcgtgattgcgcctgagcgagacgaaatacgcgatcgctgttaaaaggacaatta | |
| | caaacaggaatcgaatgcaaccggcgcaggaacactgccagcgcatcaacaatattttcac | |
| | ctgaatcaggatattcttctaatacctggaatgctgttttcccggggatcgcagtggtgag | |
| | taaccatgcatcatcaggagtacggataaaatgcttgatggtcggaagaggcataaattcc | |
| | gtcagccagtttagtctgaccatctcatctgtaacatcattggcaacgctacctttgccat | |
| | gtttcagaaacaactctggcgcatcgggcttcccatacaatcgatagattgtcgcacctga | |
| | ttgcccgacattatcgcgagcccatttatacccatataaatcagcatccatgttggaattt | |
| | aatcgcggcctcgagcaagacgtttcccgttgaatatggctcataacaccccttgtattac | |
| | tgtttatgtaagcagacagttttattgttcatgatgatatatttttatcttgtgcaatgta | |
| | acatcagagattttgaaggccaaataggccgt | |
| SP1 | atgattgacatgctagttttacgGttaccgttcatcgattctcttgtttgctccagactct | 94 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | Acaattaaaggtactctctaatcctgacctgttggagtttgcttccggtctggttcgcttt | |
| | gaGgctcgaattaaaacgAgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccccctgcaaa | |
| | aaataaattcatataaaaaacatacagataaccatctgcggtgataaattatctctggcgg | |
| | tgttgacataaataccactggcggttatactgagcacgggtaccGCCGCTGAGAAAAAGCG | |
| | AAGCGGCACTGCTCTTTAACAATTTATCAGACAATCTGTGTGGGCACTCGAAGATACGGAT | |
| | TCTTAACGTCGCAAGACGAAAAATGAATACCAAGTCTCAAGAGTGAACACGTAATTCATTA | |
| | CGAAGTTTAATTCTTTGAGCGTCAAACTTTTAAATTGAAGAGTTTGATCATGGCTCAGATT | |
| | GAACGCTGGCGGCAGGCCTAACACATGCAAGTCGAACGGTAACAGGAAGAAGCTTGCTTCT | |
| | TTGCTGACGAGTGGCGGACGGGTGAGTAATGTCTGGGAAACTGCCTGATGGAGGGGGATAA | |
| | CTACTGGAAACGGTAGCTAATACCGCATAACGTCGCAAGACCAAAGAGGGGGACCTTCGGG | |
| | CCTCTTGCCATCGGATGTGCCCAGATGGGATTAGCTAGTAGGTGGGGTAACGGCTCACCTA | |
| | GGCGACGATCCCTAGCTGGTCTGAGAGGATGACCAGCCACACTGGAACTGAGACACGGTCC | |
| | AGACTCCTACGGGAGGCAGCAGTGGGGAATATTGCACAATGGGCGCAAGCCTGATGCAGCC | |
| | ATGCCGCGTGTATGAAGAAGGCCTTCGGGTTGTAAAGTACTTTCAGCGGGGAGGAAGGGAG | |
| | TAAAGTTAATACCTTTGCTCATTGACGTTACCCGCAGAAGAAGCACCGGCTAACTCCGTGC | |
| | CAGCAGCCGCGGTAATACGGAGGGTGCAAGCGTTAATCGGAATTACTGGGCGTAAAGCGCA | |
| | CGCAGGCGGTTTGTTAAGTCAGATGTGAAATCCCCGGGCTCAACCTGGGAACTGCATCTGA | |
| | TACTGGCAAGCTTGAGTCTCGTAGAGGGGGGTAGAATTCCAGGTGTAGCGGTGAAATGCGT | |
| | AGAGATCTGGAGGAATACCGGTGGCGAAGGCGGCCCCCTGGACGAAGACTGACGCTCAGGT | |
| | GCGAAAGCGTGGGGAGCAAACAGGATTAGATACCCTGGTAGTCCACGCCGTAAACGATGTC | |
| | GACTTGGAGGTTGTGCCCTTGAGGCGTGGCTTCCGGAGCTAACGCGTTAAGTCGACCGCCT | |
| | GGGGAGTACGGCCGCAAGGTTAAAACTCAAATGAATTGACGGGGGCCCGCACAAGCGGTGG | |
| | AGCATGTGGTTTAATTCGATGCAACGCGAAGAACCTTACCTGGTCTTGACATCCACGGAAG | |
| | TTTTCAGAGATGAGAATGTGCCTTCGGGAACCGTGAGACAGGTGCTGCATGGCTGTCGTCA | |
| | GCTCGTGTTGTGAAATGTTGGGTTAAGTCCCGCAACGAGCGCAACCCTTATCCTTTGTTGC | |
| | CAGCGGTCCGGCCGGGAACTCAAAGGAGACTGCCAGTGATAAACTGGAGGAAGGTGGGGAT | |
| | GACGTCAAGTCATCATGGCCCTTACGACCAGGGCTACACACGTGCTACAATGGCGCATACA | |
| | AAGAGAAGCGACCTCGCGAGAGCAAGCGGACCTCATAAAGTGCGTCGTAGTCCGGATTGGA | |
| | GTCTGCAACTCGACTCCATGAAGTCGGAATCGCTAGTAATCGTGGATCAGAATGCCACGGT | |
| | GAATACGTTCCCGGGCCTTGTACACACCGCCCGTCACACCATGGGAGTGGGTTGCAAAAGA | |
| | AGTAGGTAGCTTAACCTTCGGGAGGGCGCTTACCACTTTGTGATTCATGACTGGGGTGAAG | |
| | TCGTAACAAGGTAACCGTAGGGGAACCTGCGGTTGGATCATGTATATACCTTAAAGAAGCG | |
| | TACTTTGTAGTGCTCACACAGATTGTCTGATAGAAAGTGAAAAGCAAGGCGTTTACGCGTT | |
| | GGGAGTGAGGCTGAAGAGAATAAGGCCGTTCGCTTTCTATTAATGAAAGCTCACCCTACAC | |
| | GAAAATATCACGCAACGCGTGATAAGCAATTTTCGTGTCCCCTTCGTCTAGAGGCCCAGGA | |
| | CACCGCCCTTTCACGGCGGTAACAGGGGTTCGAATCCCCTAGGGGACGCCACTTGCTGGTT | |
| | TGTGAGTGAAAGTCGCCGACCTTAATATCTCAAAACTCATCTTCGGGTGATGTTTGAGATA | |
| | TTTGCTCTTTAAAAATCTGGATCAAGCTGAAAATTGAAACACTGAACAACGAGAGTTGTTC | |
| | GTGAGTCTCTCAAATTTTCGCAACACGATGATGAATCGAAAGAAACATCTTCGGGTTGTGA | |
| | GGTTAAGCGACTAAGCGTACACGGTGGATGCCCTGGCAGTCAGAGGCGATGAAGGACGTGC | |
| | TAATCTGCGATAAGCGTCGGTAAGGTGATATGAACCGTTATAACCGGCGATTTCCGAATGG | |
| | GGAAACCCAGTGTGTTTCGACACACTATCATTAACTGAATCCATAGGTTAATGAGGCGAAC | |
| | CGGGGGAACTGAAACATCTAAGTACCCCGAGGAAAAGAAATCAACCGAGATTCCCCCAGTA | |
| | GCGGCGAGCGAACGGGGAGCAGCCCAGAGCCTGAATCAGTGTGTGTGTTAGTGGAAGCGTC | |
| | TGGAAAGGCGCGCGATACAGGGTGACAGCCCCGTACACAAAAATGCACATGCTGTGAGCTC | |
| | GATGAGTAGGGCGGGACACGTGGTATCCTGTCTGAATATGGGGGGACCATCCTCCAAGGCT | |
| | AAATACTCCTGACTGACCGATAGTGAACCAGTACCGTGAGGGAAAGGCGAAAAGAACCCCG | |
| | GCGAGGGGAGTGAAAAAGAACCTGAAACCGTGTACGTACAAGCAGTGGGAGCACGCTTAGG | |
| | CGTGTGACTGCGTACCTTTTGTATAATGGGTCAGCGACTTATATTCTGTAGCAAGGTTAAC | |
| | CGAATAGGGGAGCCGAAGGGAAACCGAGTCTTAACTGGGCGTTAAGTTGCAGGGTATAGAC | |
| | CCGAAACCCGGTGATCTAGCCATGGGCAGGTTGAAGGTTGGGTAACACTAACTGGAGGACC | |
| | GAACCGACTAATGTTGAAAAATTAGCGGATGACTTGTGGCTGGGGGTGAAAGGCCAATCAA | |
| | ACCGGGAGATAGCTGGTTCTCCCCGAAAGCTATTTAGGTAGCGCCTCGTGAATTCATCTCC | |
| | GGGGGTAGAGCACTGTTTCGGCAAGGGGGTCATCCCGACTTACCAACCCGATGCAAACTGC | |
| | GAATACCGGAGAATGTTATCACGGGAGACACACGGCGGGTGCTAACGTCCGTCGTGAAGAG | |
| | GGAAACAACCCAGACCGCCAGCTAAGGTCCCAAAGTCATGGTTAAGTGGGAAACGATGTGG | |
| | GAAGGCCCAGACAGCCAGGATGTTGGCTTAGAAGCAGCCATCATTTAAAGAAAGCGTAATA | |
| | GCTCACTGGTCGAGTCGGCCTGCGCGGAAGATGTAACGGGGCTAAACCATGCACCGAAGCT | |
| | GCGGCAGCGACGCTTATGCGTTGTTGGGTAGGGGAGCGTTCTGTAAGCCTGCGAAGGTGTG | |
| | CTGTGAGGCATGCTGGAGGTATCAGAAGTGCGAATGCTGACATAAGTAACGATAAAGCGGG | |
| | TGAAAAGCCCGCTCGCCGGAAGACCAAGGGTTCCTGTCCAACGTTAATCGGGGCAGGGTGA | |
| | GTCGACCCCTAAGGCGAGGCCGAAAGGCGTAGTCGATGGGAAACAGGTTAATATTCCTGTA | |
| | CTTGGTGTTACTGCGAAGGGGGGACGGAGAAGGCTATGTTGGCCGGGCGACGGTTGTCCCG | |
| | GTTTAAGCGTGTAGGCTGGTTTTCCAGGCAAATCCGGAAAATCAAGGCTGAGGCGTGATGA | |
| | CGAGGCACTACGGTGCTGAAGCAACAAATGCCCTGCTTCCAGGAAAAGCCTCTAAGCATCA | |
| | GGTAACATCAAATCGTACCCCAAACCGACACAGGTGGTCAGGTAGAGAATACCAAGGCGCT | |
| | TGAGAGAACTCGGGTGAAGGAACTAGGCAAAATGGTGCCGTAACTTCGGGAGAAGGCACGC | |
| | TGATATGTAGGTGAGGTCCCTCGCGGATGGAGCTGAAATCAGTCGAAGATACCAGCTGGCT | |
| | GCAACTGTTTATTAAAAACACAGCACTGTGCAAACACGAAAGTGGACGTATACGGTGTGAC | |
| | GCCTGCCCGGTGCCGGAAGGTTAATTGATGGGGTTAGCGCAAGCGAAGCTCTTGATCGAAG | |
| | CCCCGGTAAACGGCGGCCGTAACTATAACGGTCCTAAGGTAGCGAAATTCCTTGTCGGGTA | |
| | AGTTCCGACCTGCACGAATGGCGTAATGATGGCCAGGCTGTCTCCACCCGAGACTCAGTGA | |
| | AATTGAACTCGCTGTGAAGATGCAGTGTACCCGCGGCAAGACGGAAAGACCCCGTGAACCT | |
| | TTACTATAGCTTGACACTGAACATTGAGCCTTGATGTGTAGGATAGGTGGGAGGCTTTGAA | |
| | GTGTGGACGCCAGTCTGCATGGAGCCGACCTTGAAATACCACCCTTTAATGTTTGATGTTC | |
| | TAACGTTGACCCGTAATCCGGGTTGCGGACAGTGTCTGGTGGGTAGTTTGACTGGGGCGGT | |
| | CTCCTCCTAAAGAGTAACGGAGGAGCACGAAGGTTGGCTAATCCTGGTCGGACATCAGGAG | |
| | GTTAGTGCAATGGCATAAGCCAGCTTGACTGCGAGCGTGACGGCGCGAGCAGGTGCGAAAG | |
| | CAGGTCATAGTGATCCGGTGGTTCTGAATGGAAGGGCCATCGCTCAACGGATAAAAGGTAC | |
| | TCCGGGGATAACAGGCTGATACCGCCCAAGAGTTCATATCGACGGCGGTGTTTGGCACCTC | |
| | GATGTCGGCTCATCACATCCTGGGGCTGAAGTAGGTCCCAAGGGTATGGCTGTTCGCCATT | |
| | TAAAGTGGTACGCGAGCTGGGTTTAGAACGTCGTGAGACAGTTCGGTCCCTATCTGCCGTG | |
| | GGCGCTGGAGAACTGAGGGGGGCTGCTCCTAGTACGAGAGGACCGGAGTGGACGCATCACT | |
| | GGTGTTCGGGTTGTCATGCCAATGGCACTGCCCGGTAGCTAAATGCGGAAGAGATAAGTGC | |
| | TGAAAGCATCTAAGCACGAAACTTGCCCCGAGATGAGTTCTCCCTGACCCTTTAAGGGTCC | |
| | TGAAGGAACGTTGAAGACGACGACGTTGATAGGCCGGGTGTGTAAGCGCAGCGATGCGTTG | |
| | AGCTAACCGGTACTAATGAACCGTGAGGCTTAACCTTACAACGCCGAAGCTGTTTTGGCGG | |
| | ATGAGAGAAGATTTTCAGCCTGATACAGATTAAATCAGAACGCAGAAGCGGTCTGATAAAA | |
| | CAGAATTTGCCTGGCGGCAGTAGCGCGGTGGTCCCACCTGACCCCATGCCGAACTCAGAAG | |
| | TGAAACGCCGTAGCGCCGATGGTAGTGTGGGGTCTCCCCATGCGAGAGTAGGGAACTGCCA | |
| | GGCATCAAATAAAACGAAAGGCTCAGTCGAAAGACTGGGCCTTTCGTTTTATCTGTTGTTT | |
| | GTCGGTGAACGCTCTCCagaaggagattttcaacatgctccctcaatcggttgaatgtcgc | |
| | ccttttgtctttggcgctggtaaaccatatgaattttctattgattgtgacaaaataaact | |
| | tattccgtggtgtctttgcgtttcttttatatgttgccacctttatgtatgtatCttctac | |
| | gtttgctaacatactgcgtaataaggagtcttaatcatgccagttcttttgggtattccgt | |
| | tattattgcgtttcctcggtttccttctggtaactttgttcggctatctgcttacttttct | |
| | Caaaaagggcttcggtaagatagctattgctatttcattgtttcttgctcttattattggg | |
| | cttaactcaattcttgtgggttatctctctgatattagTgctcaattaccctctgactttg | |
| | ttcagggtgttcagttaattctcccgtctaatgcgcttccctgtttttatgttattctctc | |
| | tgtaaagActgctattttcatttttgacgttaaacaaaaaatcgtttcttatttggattgg | |
| | gaCaaataatatggctgtttattttgtaactggcaaattaggctctggaaagacgctcgtt | |
| | agcgttggtaagattcaggataaaattgtagctgggtgcaaaatagcaactaatcttgatt | |
| | taaggcttcaaaacctcccgcaagtcgggaggttcgctaaaacgcctcgcgttcttagaat | |
| | accggataagccttctatatctgatttgcttgctattgggcgcggtaatgattcctacgat | |
| | gaaaataaaaacggAttgcttgttctcgatgagtgcggtacttggtttaatacccgttctt | |
| | ggaatgataaggaaagacagccgattattgattggtttctacatgctcgtaaattaggatg | |
| | ggatattatttttcttgttcaggacttatctattgttgataaacaggcgcgttctgcatta | |
| | gctgaacatgttgtttattgtcgtcgtctggacagaattactttaccttttgtcggtactt | |
| | tatattctcttattactggctcgaaaatgcctctgcctaaattacatgttggcgttgttaa | |
| | atatggcgattctcaattaagccctactgttgagcgttggctttatactggtaagaatttg | |
| | tataacgcataCgatactaaacaggctttttctagtaattatgattccggtgtttattctt | |
| | atttaacgccttatttatcacacggtcggtatttcaaaccattaaatttaggtcagaagat | |
| | gaaattaactaaaatatatttgaaaaagttttctcgcgttctttgtcttgcgattggattt | |
| | gcatcagcatttacatatagttatataacccaacctaagccggaggttaaaaaggtagtct | |
| | ctcagacctatgattttgataaattcactattgactcttctcaTcgtcttaatctaagcta | |
| | tcgctatgttttcaaggattctaagggaaaattaattaatagcgacgatttacagaagcaa | |
| | ggttattcactTacatatattgatttatgtactgtttccattaaaaaaggtaattcaaatg | |
| | aaattgttaaatgtaattaattttgttttcttgatgtttgtttcatcatcttcttttgctc | |
| | aggtaattgaaatgaataattcgcctctgcgcgattttAtaacttggtattcaaagcaatc | |
| | aggcgaatccgttattgtttctcccgatgtaaaaggtactgttactgtatattcatctgac | |
| | gttaaacctgaaaatctacgcaatttctttatttctgttttacgtgcaaataattttgata | |
| | tggtaggttctaacccttccattattcagaagtataatccaaacaatcaggattatattga | |
| | tgaattgccatcaCctgataatcaggaatatgatgataattccgctccttctggtggtttc | |
| | tttgtCccgcaaaatgataatgttactcaaacttttaaaattaataacgttcgggcaaagg | |
| | atttaatacgagttgtcgaattgtttgtaaagtctaatacttctaaatcctcaaatgtatt | |
| | atctattgacggAtctaatctattagttgttagtgctcctaaagatattttagataacctt | |
| | TctcaattcctttcaactgttgatttgccaactgaccagGtattgattgagggtttgatat | |
| | ttgaggttcagcaaggtgatgctttagatttttcatttgctgctggctctcagcgtggcac | |
| | tgttgcaggcggAgttaatactgaccgcctcacctctgttttatcttctgctggtggttcg | |
| | ttcggtatttttaatggcgatgttttagggctatcagttcgcgcattaaagactaatagcc | |
| | attcaaaaatattgtctgtgccacgtattcttacgctttcaggtcagaagggttctatctc | |
| | tAttggccagaatgtcccttttattactggtcgtgtAactggtgaatctgccaatgtaaat | |
| | aatccatttcagacgattgagcgtcaaaatgtaggtatttccatgagcgtttttcctgttg | |
| | caatggctggcggtaatattgttctggatattaccagcaaggccgatagtttgagttcttc | |
| | tactcaggcaagtgatgttattactaaCcaaagaagtattgctacaacggttaatttgcgt | |
| | gatggacagactcttCtactcggtggcctcactgattataaaaacacttctcaggattctg | |
| | gcgtaccgttcctgtctaaaatccctttaatcggcctcctgtttagctcccgctctgattc | |
| | taacgaggaGagcacgttatacgtgctcgtcaaagcaaccatagtacgcgccctgtagcgg | |
| | cgcattaagcgcggcgggtgtggtggttacgcgcagcgtgaccgctacacttgccagcgcc | |
| | ctagcgcccgctcctttcgctttcttcccttcctttctcgccacgttcgccggctttcccc | |
| | gtcaagcGctaaatcgggggcccctttagggttccgatttagtgctttacggcacctcgac | |
| | cccaaaaaacttgatttgggtgatggttcacgtagtgggccatcgccctgatagacggttt | |
| | ttcgccctttgacgttggagtccacgttctttaatagtggactcttgttccaaactggaac | |
| | aacactcaaccctatctcgggctattcttttgatttataagggattttgccgatttcggcc | |
| | tattggttaaaaaatgaActgatttaacaaaaatttaacgcgaattttaacaaaatattaa | |
| | cgtttacaatttaaatatttgcttatacaatcttcctgtCttGggggcttttcttattatc | |
| | aaccggggtacat | |
| SP2 | atgattgacatgctagttttacgGttaccgttcatcgattctcttgtttgctccagactct | 95 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatA | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatGCTGAGAAAAAGCGAAGCGGCACTGCTCTTTA | |
| | ACAATTTATCAGACAATCTGTGTGGGCACTCGAAGATACGGATTCTTAACGTCGCAAGACG | |
| | AAAAATGAATACCAAGTCTCAAGAGTGAACACGTAATTCATTACGAAGTTTAATTCTTTGA | |
| | GCGTCAAACTTTTAAATTGAAGAGTTTGATCATGGCTCAGATTGAACGCTGGCGGCAGGCC | |
| | TAACACATGCAAGTCGAACGGTAACAGGAAGAAGCTTGCTTCTTTGCTGACGAGTGGCGGA | |
| | CGGGTGAGTAATGTCTGGGAAACTGCCTGATGGAGGGGGATAACTACTGGAAACGGTAGCT | |
| | AATACCGCATAACGTCGCAAGACCAAAGAGGGGGACCTTCGGGCCTCTTGCCATCGGATGT | |
| | GCCCAGATGGGATTAGCTAGTAGGTGGGGTAACGGCTCACCTAGGCGACGATCCCTAGCTG | |
| | GTCTGAGAGGATGACCAGCCACACTGGAACTGAGACACGGTCCAGACTCCTACGGGAGGCA | |
| | GCAGTGGGGAATATTGCACAATGGGCGCAAGCCTGATGCAGCCATGCCGCGTGTATGAAGA | |
| | AGGCCTTCGGGTTGTAAAGTACTTTCAGCGGGGAGGAAGGGAGTAAAGTTAATACCTTTGC | |
| | TCATTGACGTTACCCGCAGAAGAAGCACCGGCTAACTCCGTGCCAGCAGCCGCGGTAATAC | |
| | GGAGGGTGCAAGCGTTAATCGGAATTACTGGGCGTAAAGCGCACGCAGGCGGTTTGTTAAG | |
| | TCAGATGTGAAATCCCCGGGCTCAACCTGGGAACTGCATCTGATACTGGCAAGCTTGAGTC | |
| | TCGTAGAGGGGGGTAGAATTCCAGGTGTAGCGGTGAAATGCGTAGAGATCTGGAGGAATAC | |
| | CGGTGGCGAAGGCGGCCCCCTGGACGAAGACTGACGCTCAGGTGCGAAAGCGTGGGGAGCA | |
| | AACAGGATTAGATACCCTGGTAGTCCACGCCGTAAACGATGTCGACTTGGAGGTTGTGCCC | |
| | TTGAGGCGTGGCTTCCGGAGCTAACGCGTTAAGTCGACCGCCTGGGGAGTACGGCCGCAAG | |
| | GTTAAAACTCAAATGAATTGACGGGGGCCCGCACAAGCGGTGGAGCATGTGGTTTAATTCG | |
| | ATGCAACGCGAAGAACCTTACCTGGTCTTGACATCCACGGAAGTTTTCAGAGATGAGAATG | |
| | TGCCTTCGGGAACCGTGAGACAGGTGCTGCATGGCTGTCGTCAGCTCGTGTTGTGAAATGT | |
| | TGGGTTAAGTCCCGCAACGAGCGCAACCCTTATCCTTTGTTGCCAGCGGTCCGGCCGGGAA | |
| | CTCAAAGGAGACTGCCAGTGATAAACTGGAGGAAGGTGGGGATGACGTCAAGTCATCATGG | |
| | CCCTTACGACCAGGGCTACACACGTGCTACAATGGCGCATACAAAGAGAAGCGACCTCGCG | |
| | AGAGCAAGCGGACCTCATAAAGTGCGTCGTAGTCCGGATTGGAGTCTGCAACTCGACTCCA | |
| | TGAAGTCGGAATCGCTAGTAATCGTGGATCAGAATGCCACGGTGAATACGTTCCCGGGCCT | |
| | TGTACACACCGCCCGTCACACCATGGGAGTGGGTTGCAAAAGAAGTAGGTAGCTTAACCTT | |
| | CGGGAGGGCGCTTACCACTTTGTGATTCATGACTGGGGTGAAGTCGTAACAAGGTAACCGT | |
| | AGGGGAACCTGCGGTTGGATCATGTATATACCTTAAAGAAGCGTACTTTGTAGTGCTCACA | |
| | CAGATTGTCTGATAGAAAGTGAAAAGCAAGGCGTTTACGCGTTGGGAGTGAGGCagaagga | |
| | gattttcaacatgctccctcaatcggttgaatgtcgcccttttgtctttggcgctggtaaa | |
| | ccatatgaattttctattgattgtgacaaaataaacttattccgtggtgtctttgcgtttc | |
| | ttttatatgttgccacctttatgtatgtatCttctacgtttgctaacatactgcgtaataa | |
| | ggagtcttaatcatgccagttcttttgggtattccgttattattgcgtttcctcggtttcc | |
| | ttctggtaactttgttcggctatctgcttacttttctCaaaaagggcttcggtaagatagc | |
| | tattgctatttcattgtttcttgctcttattattgggcttaactcaattcttgtgggttat | |
| | ctctctgatattagTgctcaattaccctctgactttgttcagggtgttcagttaattctcc | |
| | cgtctaatgcgcttccctgtttttatgttattctctctgtaaagActgctattttcatttt | |
| | tgacgttaaacaaaaaatcgtttcttatttggattgggaCaaataatatggctgtttattt | |
| | tgtaactggcaaattaggctctggaaagacgctcgttagcgttggtaagattcaggataaa | |
| | attgtagctgggtgcaaaatagcaactaatcttgatttaaggcttcaaaacctcccgcaag | |
| | tcgggaggttcgctaaaacgcctcgcgttcttagaataccggataagccttctatatctga | |
| | tttgcttgctattgggcgcggtaatgattcctacgatgaaaataaaaacggAttgcttgtt | |
| | ctcgatgagtgcggtacttggtttaatacccgttcttggaatgataaggaaagacagccga | |
| | ttattgattggtttctacatgctcgtaaattaggatgggatattatttttcttgttcagga | |
| | cttatctattgttgataaacaggcgcgttctgcattagctgaacatgttgtttattgtcgt | |
| | cgtctggacagaattactttaccttttgtcggtactttatattctcttattactggctcga | |
| | aaatgcctctgcctaaattacatgttggcgttgttaaatatggcgattctcaattaagccc | |
| | tactgttgagcgttggctttatactggtaagaatttgtataacgcataCgatactaaacag | |
| | gctttttctagtaattatgattccggtgtttattcttatttaacgccttatttatcacacg | |
| | gtcggtatttcaaaccattaaatttaggtcagaagatgaaattaactaaaatatatttgaa | |
| | aaagttttctcgcgttctttgtcttgcgattggatttgcatcagcatttacatatagttat | |
| | ataacccaacctaagccggaggttaaaaaggtagtctctcagacctatgattttgataaat | |
| | tcactattgactcttctcaTcgtcttaatctaagctatcgctatgttttcaaggattctaa | |
| | gggaaaattaattaatagcgacgatttacagaagcaaggttattcactTacatatattgat | |
| | ttatgtactgtttccattaaaaaaggtaattcaaatgaaattgttaaatgtaattaatttt | |
| | gttttcttgatgtttgtttcatcatcttcttttgctcaggtaattgaaatgaataattcgc | |
| | ctctgcgcgattttAtaacttggtattcaaagcaatcaggcgaatccgttattgtttctcc | |
| | cgatgtaaaaggtactgttactgtatattcatctgacgttaaacctgaaaatctacgcaat | |
| | ttctttatttctgttttacgtgcaaataattttgatatggtaggttctaacccttccatta | |
| | ttcagaagtataatccaaacaatcaggattatattgatgaattgccatcaCctgataatca | |
| | ggaatatgatgataattccgctccttctggtggtttctttgtCccgcaaaatgataatgtt | |
| | actcaaacttttaaaattaataacgttcgggcaaaggatttaatacgagttgtcgaattgt | |
| | ttgtaaagtctaatacttctaaatcctcaaatgtattatctattgacggAtctaatctatt | |
| | agttgttagtgctcctaaagatattttagataaccttTctcaattcctttcaactgttgat | |
| | ttgccaactgaccagGtattgattgagggtttgatatttgaggttcagcaaggtgatgctt | |
| | tagatttttcatttgctgctggctctcagcgtggcactgttgcaggcggAgttaatactga | |
| | ccgcctcacctctgttttatcttctgctggtggttcgttcggtatttttaatggcgatgtt | |
| | ttagggctatcagttcgcgcattaaagactaatagccattcaaaaatattgtctgtgccac | |
| | gtattcttacgctttcaggtcagaagggttctatctctAttggccagaatgtcccttttat | |
| | tactggtcgtgtAactggtgaatctgccaatgtaaataatccatttcagacgattgagcgt | |
| | caaaatgtaggtatttccatgagcgtttttcctgttgcaatggctggcggtaatattgttc | |
| | tggatattaccagcaaggccgatagtttgagttcttctactcaggcaagtgatgttattac | |
| | taaCcaaagaagtattgctacaacggttaatttgcgtgatggacagactcttCtactcggt | |
| | ggcctcactgattataaaaacacttctcaggattctggcgtaccgttcctgtctaaaatcc | |
| | ctttaatcggcctcctgtttagctcccgctctgattctaacgaggaGagcacgttatacgt | |
| | gctcgtcaaagcaaccatagtacgcgccctgtagcggcgcattaagcgcggcgggtgtggt | |
| | ggttacgcgcagcgtgaccgctacacttgccagcgccctagcgcccgctcctttcgctttc | |
| | ttcccttcctttctcgccacgttcgccggctttccccgtcaagcGctaaatcgggggcccc | |
| | tttagggttccgatttagtgctttacggcacctcgaccccaaaaaacttgatttgggtgat | |
| | ggttcacgtagtgggccatcgccctgatagacggtttttcgccctttgacgttggagtcca | |
| | cgttctttaatagtggactcttgttccaaactggaacaacactcaaccctatctcgggcta | |
| | ttcttttgatttataagggattttgccgatttcggcctattggttaaaaaatgaActgatt | |
| | taacaaaaatttaacgcgaattttaacaaaatattaacgtttacaatttaaatatttgctt | |
| | atacaatcttcctgtCttGggggcttttcttattatcaaccggggtacat | |
| SP_{B} | atgattgacatgctagttttacgGttaccgttcatcgattctcttgtttgctccagactct | 96 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | Acaattaaaggtactctctaatcctgacctgttggagtttgcttccggtctggttcgcttt | |
| | gaGgctcgaattaaaacgAgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccccctgcaaa | |
| | aaataaattcatataaaaaacatacagataaccatctgcggtgataaattatctctggcgg | |
| | tgttgacataaataccactggcggttatactgagcacgggtaccGCCGCTGAGAAAAAGCG | |
| | AAGCGGCACTGCTCTTTAACAATTTATCAGACAATCTGTGTGGGCACTCGAAGATACGGAT | |
| | TCTTAACGTCGCAAGACGAAAAATGAATACCAAGTCTCAAGAGTGAACACGTAATTCATTA | |
| | CGAAGTTTAATTCTTTGAGCGTCAAACTTTTAAATTGAAGAGTTTGATCATGGCTCAGATT | |
| | GAACGCTGGCGGCAGGCCTAACACATGCAAGTCGAACGGTAACAGGAAGAAGCTTGCTTCT | |
| | TTGCTGACGAGTGGCGGACGGGTGAGTAATGTCTGGGAAACTGCCTGATGGAGGGGGATAA | |
| | CTACTGGAAACGGTAGCTAATACCGCATAACGTCGCAAGACCAAAGAGGGGGACCTTCGGG | |
| | CCTCTTGCCATCGGATGTGCCCAGATGGGATTAGCTAGTAGGTGGGGTAACGGCTCACCTA | |
| | GGCGACGATCCCTAGCTGGTCTGAGAGGATGACCAGCCACACTGGAACTGAGACACGGTCC | |
| | AGACTCCTACGGGAGGCAGCAGTGGGGAATATTGCACAATGGGCGCAAGCCTGATGCAGCC | |
| | ATGCCGCGTGTATGAAGAAGGCCTTCGGGTTGTAAAGTACTTTCAGCGGGGAGGAAGGGAG | |
| | TAAAGTTAATACCTTTGCTCATTGACGTTACCCGCAGAAGAAGCACCGGCTAACTCCGTGC | |
| | CAGCAGCCGCGGTAATACGGAGGGTGCAAGCGTTAATCGGAATTACTGGGCGTAAAGCGCA | |
| | CGCAGGCGGTTTGTTAAGTCAGATGTGAAATCCCCGGGCTCAACCTGGGAACTGCATCTGA | |
| | TACTGGCAAGCTTGAGTCTCGTAGAGGGGGGTAGAATTCCAGGTGTAGCGGTGAAATGCGT | |
| | AGAGATCTGGAGGAATACCGGTGGCGAAGGCGGCCCCCTGGACGAAGACTGACGCTCAGGT | |
| | GCGAAAGCGTGGGGAGCAAACAGGATTAGATACCCTGGTAGTCCACGCCGTAAACGATGTC | |
| | GACTTGGAGGTTGTGCCCTTGAGGCGTGGCTTCCGGAGCTAACGCGTTAAGTCGACCGCCT | |
| | GGGGAGTACGGCCGCAAGGTTAAAACTCAAATGAATTGACGGGGGCCCGCACAAGCGGTGG | |
| | AGCATGTGGTTTAATTCGATGCAACGCGAAGAACCTTACCTGGTCTTGACATCCACGGAAG | |
| | TTTTCAGAGATGAGAATGTGCCTTCGGGAACCGTGAGACAGGTGCTGCATGGCTGTCGTCA | |
| | GCTCGTGTTGTGAAATGTTGGGTTAAGTCCCGCAACGAGCGCAACCCTTATCCTTTGTTGC | |
| | CAGCGGTCCGGCCGGGAACTCAAAGGAGACTGCCAGTGATAAACTGGAGGAAGGTGGGGAT | |
| | GACGTCAAGTCATCATGGCCCTTACGACCAGGGCTACACACGTGCTACAATGGCGCATACA | |
| | AAGAGAAGCGACCTCGCGAGAGCAAGCGGACCTCATAAAGTGCGTCGTAGTCCGGATTGGA | |
| | GTCTGCAACTCGACTCCATGAAGTCGGAATCGCTAGTAATCGTGGATCAGAATGCCACGGT | |
| | GAATACGTTCCCGGGCCTTGTACACACCGCCCGTCACACCATGGGAGTGGGTTGCAAAAGA | |
| | AGTAGGTAGCTTAACCTTCGGGAGGGCGCTTACCACTTTGTGATTCATGACTGGGGTGAAG | |
| | TCGTAACAAGGTAACCGTAGGGGAACCTGCGGTTGGATCATGTGGTTACCTTAAAGAAGCG | |
| | TACTTTGTAGTGCTCACACAGATTGTCTGATAGAAAGTGAAAAGCAAGGCGTTTACGCGTT | |
| | GGGAGTGAGGCTGAAGAGAATAAGGCCGTTCGCTTTCTATTAATGAAAGCTCACCCTACAC | |
| | GAAAATATCACGCAACGCGTGATAAGCAATTTTCGTGTCCCCTTCGTCTAGAGGCCCAGGA | |
| | CACCGCCCTTTCACGGCGGTAACAGGGGTTCGAATCCCCTAGGGGACGCCACTTGCTGGTT | |
| | TGTGAGTGAAAGTCGCCGACCTTAATATCTCAAAACTCATCTTCGGGTGATGTTTGAGATA | |
| | TTTGCTCTTTAAAAATCTGGATCAAGCTGAAAATTGAAACACTGAACAACGAGAGTTGTTC | |
| | GTGAGTCTCTCAAATTTTCGCAACACGATGATGAATCGAAAGAAACATCTTCGGGTTGTGA | |
| | GGTTAAGCGACTAAGCGTACACGGTGGATGCCCTGGCAGTCAGAGGCGATGAAGGACGTGC | |
| | TAATCTGCGATAAGCGTCGGTAAGGTGATATGAACCGTTATAACCGGCGATTTCCGAATGG | |
| | GGAAACCCAGTGTGTTTCGACACACTATCATTAACTGAATCCATAGGTTAATGAGGCGAAC | |
| | CGGGGGAACTGAAACATCTAAGTACCCCGAGGAAAAGAAATCAACCGAGATTCCCCCAGTA | |
| | GCGGCGAGCGAACGGGGAGCAGCCCAGAGCCTGAATCAGTGTGTGTGTTAGTGGAAGCGTC | |
| | TGGAAAGGCGCGCGATACAGGGTGACAGCCCCGTACACAAAAATGCACATGCTGTGAGCTC | |
| | GATGAGTAGGGCGGGACACGTGGTATCCTGTCTGAATATGGGGGGACCATCCTCCAAGGCT | |
| | AAATACTCCTGACTGACCGATAGTGAACCAGTACCGTGAGGGAAAGGCGAAAAGAACCCCG | |
| | GCGAGGGGAGTGAAAAAGAACCTGAAACCGTGTACGTACAAGCAGTGGGAGCACGCTTAGG | |
| | CGTGTGACTGCGTACCTTTTGTATAATGGGTCAGCGACTTATATTCTGTAGCAAGGTTAAC | |
| | CGAATAGGGGAGCCGAAGGGAAACCGAGTCTTAACTGGGCGTTAAGTTGCAGGGTATAGAC | |
| | CCGAAACCCGGTGATCTAGCCATGGGCAGGTTGAAGGTTGGGTAACACTAACTGGAGGACC | |
| | GAACCGACTAATGTTGAAAAATTAGCGGATGACTTGTGGCTGGGGGTGAAAGGCCAATCAA | |
| | ACCGGGAGATAGCTGGTTCTCCCCGAAAGCTATTTAGGTAGCGCCTCGTGAATTCATCTCC | |
| | GGGGGTAGAGCACTGTTTCGGCAAGGGGGTCATCCCGACTTACCAACCCGATGCAAACTGC | |
| | GAATACCGGAGAATGTTATCACGGGAGACACACGGCGGGTGCTAACGTCCGTCGTGAAGAG | |
| | GGAAACAACCCAGACCGCCAGCTAAGGTCCCAAAGTCATGGTTAAGTGGGAAACGATGTGG | |
| | GAAGGCCCAGACAGCCAGGATGTTGGCTTAGAAGCAGCCATCATTTAAAGAAAGCGTAATA | |
| | GCTCACTGGTCGAGTCGGCCTGCGCGGAAGATGTAACGGGGCTAAACCATGCACCGAAGCT | |
| | GCGGCAGCGACGCTTATGCGTTGTTGGGTAGGGGAGCGTTCTGTAAGCCTGCGAAGGTGTG | |
| | CTGTGAGGCATGCTGGAGGTATCAGAAGTGCGAATGCTGACATAAGTAACGATAAAGCGGG | |
| | TGAAAAGCCCGCTCGCCGGAAGACCAAGGGTTCCTGTCCAACGTTAATCGGGGCAGGGTGA | |
| | GTCGACCCCTAAGGCGAGGCCGAAAGGCGTAGTCGATGGGAAACAGGTTAATATTCCTGTA | |
| | CTTGGTGTTACTGCGAAGGGGGGACGGAGAAGGCTATGTTGGCCGGGCGACGGTTGTCCCG | |
| | GTTTAAGCGTGTAGGCTGGTTTTCCAGGCAAATCCGGAAAATCAAGGCTGAGGCGTGATGA | |
| | CGAGGCACTACGGTGCTGAAGCAACAAATGCCCTGCTTCCAGGAAAAGCCTCTAAGCATCA | |
| | GGTAACATCAAATCGTACCCCAAACCGACACAGGTGGTCAGGTAGAGAATACCAAGGCGCT | |
| | TGAGAGAACTCGGGTGAAGGAACTAGGCAAAATGGTGCCGTAACTTCGGGAGAAGGCACGC | |
| | TGATATGTAGGTGAGGTCCCTCGCGGATGGAGCTGAAATCAGTCGAAGATACCAGCTGGCT | |
| | GCAACTGTTTATTAAAAACACAGCACTGTGCAAACACGAAAGTGGACGTATACGGTGTGAC | |
| | GCCTGCCCGGTGCCGGAAGGTTAATTGATGGGGTTAGCGCAAGCGAAGCTCTTGATCGAAG | |
| | CCCCGGTAAACGGCGGCCGTAACTATAACGGTCCTAAGGTAGCGAAATTCCTTGTCGGGTA | |
| | AGTTCCGACCTGCACGAATGGCGTAATGATGGCCAGGCTGTCTCCACCCGAGACTCAGTGA | |
| | AATTGAACTCGCTGTGAAGATGCAGTGTACCCGCGGCAAGACGGAAAGACCCCGTGAACCT | |
| | TTACTATAGCTTGACACTGAACATTGAGCCTTGATGTGTAGGATAGGTGGGAGGCTTTGAA | |
| | GTGTGGACGCCAGTCTGCATGGAGCCGACCTTGAAATACCACCCTTTAATGTTTGATGTTC | |
| | TAACGTTGACCCGTAATCCGGGTTGCGGACAGTGTCTGGTGGGTAGTTTGACTGGGGCGGT | |
| | CTCCTCCTAAAGAGTAACGGAGGAGCACGAAGGTTGGCTAATCCTGGTCGGACATCAGGAG | |
| | GTTAGTGCAATGGCATAAGCCAGCTTGACTGCGAGCGTGACGGCGCGAGCAGGTGCGAAAG | |
| | CAGGTCATAGTGATCCGGTGGTTCTGAATGGAAGGGCCATCGCTCAACGGATAAAAGGTAC | |
| | TCCGGGGATAACAGGCTGATACCGCCCAAGAGTTCATATCGACGGCGGTGTTTGGCACCTC | |
| | GATGTCGGCTCATCACATCCTGGGGCTGAAGTAGGTCCCAAGGGTATGGCTGTTCGCCATT | |
| | TAAAGTGGTACGCGAGCTGGGTTTAGAACGTCGTGAGACAGTTCGGTCCCTATCTGCCGTG | |
| | GGCGCTGGAGAACTGAGGGGGGCTGCTCCTAGTACGAGAGGACCGGAGTGGACGCATCACT | |
| | GGTGTTCGGGTTGTCATGCCAATGGCACTGCCCGGTAGCTAAATGCGGAAGAGATAAGTGC | |
| | TGAAAGCATCTAAGCACGAAACTTGCCCCGAGATGAGTTCTCCCTGACCCTTTAAGGGTCC | |
| | TGAAGGAACGTTGAAGACGACGACGTTGATAGGCCGGGTGTGTAAGCGCAGCGATGCGTTG | |
| | AGCTAACCGGTACTAATGAACCGTGAGGCTTAACCTTACAACGCCGAAGCTGTTTTGGCGG | |
| | ATGAGAGAAGATTTTCAGCCTGATACAGATTAAATCAGAACGCAGAAGCGGTCTGATAAAA | |
| | CAGAATTTGCCTGGCGGCAGTAGCGCGGTGGTCCCACCTGACCCCATGCCGAACTCAGAAG | |
| | TGAAACGCCGTAGCGCCGATGGTAGTGTGGGGTCTCCCCATGCGAGAGTAGGGAACTGCCA | |
| | GGCATCAAATAAAACGAAAGGCTCAGTCGAAAGACTGGGCCTTTCGTTTTATCTGTTGTTT | |
| | GTCGGTGAACGCTCTCCagaaggagattttcaacatgctccctcaatcggttgaatgtcgc | |
| | ccttttgtctttggcgctggtaaaccatatgaattttctattgattgtgacaaaataaact | |
| | tattccgtggtgtctttgcgtttcttttatatgttgccacctttatgtatgtatCttctac | |
| | gtttgctaacatactgcgtaataaggagtcttaatcatgccagttcttttgggtattccgt | |
| | tattattgcgtttcctcggtttccttctggtaactttgttcggctatctgcttacttttct | |
| | Caaaaagggcttcggtaagatagctattgctatttcattgtttcttgctcttattattggg | |
| | cttaactcaattcttgtgggttatctctctgatattagTgctcaattaccctctgactttg | |
| | ttcagggtgttcagttaattctcccgtctaatgcgcttccctgtttttatgttattctctc | |
| | tgtaaagActgctattttcatttttgacgttaaacaaaaaatcgtttcttatttggattgg | |
| | gaCaaataatatggctgtttattttgtaactggcaaattaggctctggaaagacgctcgtt | |
| | agcgttggtaagattcaggataaaattgtagctgggtgcaaaatagcaactaatcttgatt | |
| | taaggcttcaaaacctcccgcaagtcgggaggttcgctaaaacgcctcgcgttcttagaat | |
| | accggataagccttctatatctgatttgcttgctattgggcgcggtaatgattcctacgat | |
| | gaaaataaaaacggAttgcttgttctcgatgagtgcggtacttggtttaatacccgttctt | |
| | ggaatgataaggaaagacagccgattattgattggtttctacatgctcgtaaattaggatg | |
| | ggatattatttttcttgttcaggacttatctattgttgataaacaggcgcgttctgcatta | |
| | gctgaacatgttgtttattgtcgtcgtctggacagaattactttaccttttgtcggtactt | |
| | tatattctcttattactggctcgaaaatgcctctgcctaaattacatgttggcgttgttaa | |
| | atatggcgattctcaattaagccctactgttgagcgttggctttatactggtaagaatttg | |
| | tataacgcataCgatactaaacaggctttttctagtaattatgattccggtgtttattctt | |
| | atttaacgccttatttatcacacggtcggtatttcaaaccattaaatttaggtcagaagat | |
| | gaaattaactaaaatatatttgaaaaagttttctcgcgttctttgtcttgcgattggattt | |
| | gcatcagcatttacatatagttatataacccaacctaagccggaggttaaaaaggtagtct | |
| | ctcagacctatgattttgataaattcactattgactcttctcaTcgtcttaatctaagcta | |
| | tcgctatgttttcaaggattctaagggaaaattaattaatagcgacgatttacagaagcaa | |
| | ggttattcactTacatatattgatttatgtactgtttccattaaaaaaggtaattcaaatg | |
| | aaattgttaaatgtaattaattttgttttcttgatgtttgtttcatcatcttcttttgctc | |
| | aggtaattgaaatgaataattcgcctctgcgcgattttAtaacttggtattcaaagcaatc | |
| | aggcgaatccgttattgtttctcccgatgtaaaaggtactgttactgtatattcatctgac | |
| | gttaaacctgaaaatctacgcaatttctttatttctgttttacgtgcaaataattttgata | |
| | tggtaggttctaacccttccattattcagaagtataatccaaacaatcaggattatattga | |
| | tgaattgccatcaCctgataatcaggaatatgatgataattccgctccttctggtggtttc | |
| | tttgtCccgcaaaatgataatgttactcaaacttttaaaattaataacgttcgggcaaagg | |
| | atttaatacgagttgtcgaattgtttgtaaagtctaatacttctaaatcctcaaatgtatt | |
| | atctattgacggAtctaatctattagttgttagtgctcctaaagatattttagataacctt | |
| | TctcaattcctttcaactgttgatttgccaactgaccagGtattgattgagggtttgatat | |
| | ttgaggttcagcaaggtgatgctttagatttttcatttgctgctggctctcagcgtggcac | |
| | tgttgcaggcggAgttaatactgaccgcctcacctctgttttatcttctgctggtggttcg | |
| | ttcggtatttttaatggcgatgttttagggctatcagttcgcgcattaaagactaatagcc | |
| | attcaaaaatattgtctgtgccacgtattcttacgctttcaggtcagaagggttctatctc | |
| | tAttggccagaatgtcccttttattactggtcgtgtAactggtgaatctgccaatgtaaat | |
| | aatccatttcagacgattgagcgtcaaaatgtaggtatttccatgagcgtttttcctgttg | |
| | caatggctggcggtaatattgttctggatattaccagcaaggccgatagtttgagttcttc | |
| | tactcaggcaagtgatgttattactaaCcaaagaagtattgctacaacggttaatttgcgt | |
| | gatggacagactcttCtactcggtggcctcactgattataaaaacacttctcaggattctg | |
| | gcgtaccgttcctgtctaaaatccctttaatcggcctcctgtttagctcccgctctgattc | |
| | taacgaggaGagcacgttatacgtgctcgtcaaagcaaccatagtacgcgccctgtagcgg | |
| | cgcattaagcgcggcgggtgtggtggttacgcgcagcgtgaccgctacacttgccagcgcc | |
| | ctagcgcccgctcctttcgctttcttcccttcctttctcgccacgttcgccggctttcccc | |
| | gtcaagcGctaaatcgggggcccctttagggttccgatttagtgctttacggcacctcgac | |
| | cccaaaaaacttgatttgggtgatggttcacgtagtgggccatcgccctgatagacggttt | |
| | ttcgccctttgacgttggagtccacgttctttaatagtggactcttgttccaaactggaac | |
| | aacactcaaccctatctcgggctattcttttgatttataagggattttgccgatttcggcc | |
| | tattggttaaaaaatgaActgatttaacaaaaatttaacgcgaattttaacaaaatattaa | |
| | cgtttacaatttaaatatttgcttatacaatcttcctgtCttGggggcttttcttattatc | |
| | aaccggggtacat | |
| SP_{H3} | atgattgacatgctagttttacgGttaccgttcatcgattctcttgtttgctccagactct | 97 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatA | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatGCTGAGAAAAAGCGAAGCGGCACTGCTCTTTA | |
| | ACAATTTATCAGACAATCTGTGTGGGCACTCGAAGATACGGATTCTTAACGTCGCAAGACG | |
| | AAAAATGAATACCAAGTCTCAAGAGTGAACACGTAATTCATTACGAAGTTTAATTCTTTGA | |
| | GCGTCAAACTTTTAAATTGAAGAGTTTGATCATGGCTCAGATTGAACGCTGGCGGCAGGCC | |
| | TAACACATGCAAGTCGAACGGTAACAGGAAGAAGCTTGCTTCTTTGCTGACGAGTGGCGGA | |
| | CGGGTGAGTAATGTCTGGGAAACTGCCTGATGGAGGGGGATAACTACTGGAAACGGTAGCT | |
| | AATACCGCATAACGTCGCAAGACCAAAGAGGGGGACCTTCGGGCCTCTTGCCATCGGATGT | |
| | GCCCAGATGGGATTAGCTAGTAGGTGGGGTAACGGCTCACCTAGGCGACGATCCCTAGCTG | |
| | GTCTGAGAGGATGACCAGCCACACTGGAACTGAGACACGGTCCAGACTCCTACGGGAGGCA | |
| | GCAGTGGGGAATATTGCACAATGGGCGCAAGCCTGATGCAGCCATGCCGCGTGTATGAAGA | |
| | AGGCCTTCGGGTTGTAAAGTACTTTCAGCGGGGAGGAAGGGAGTAAAGTTAATACCTTTGC | |
| | TCATTGACGTTACCCGCAGAAGAAGCACCGGCTAACTCCGTGCCAGCAGCCGCGGTAATAC | |
| | GGAGGGTGCAAGCGTTAATCGGAATTACTGGGCGTAAAGCGCACGCAGGCGGTTTGTTAAG | |
| | TCAGATGTGAAATCCCCGGGCTCAACCTGGGAACTGCATCTGATACTGGCAAGCTTGAGTC | |
| | TCGTAGAGGGGGGTAGAATTCCAGGTGTAGCGGTGAAATGCGTAGAGATCTGGAGGAATAC | |
| | CGGTGGCGAAGGCGGCCCCCTGGACGAAGACTGACGCTCAGGTGCGAAAGCGTGGGGAGCA | |
| | AACAGGATTAGATACCCTGGTAGTCCACGCCGTAAACGATGTCGACTTGGAGGTTGTGCCC | |
| | TTGAGGCGTGGCTTCCGGAGCTAACGCGTTAAGTCGACCGCCTGGGGAGTACGGCCGCAAG | |
| | GTTAAAACTCAAATGAATTGACGGGGGCCCGCACAAGCGGTGGAGCATGTGGTTTAATTCG | |
| | ATGCAACGCGAAGAACCTTACCTGGTCTTGACATCCACGGAAGTTTTCAGAGATGAGAATG | |
| | TGCCTTCGGGAACCGTGAGACAGGTGCTGCATGGCTGTCGTCAGCTCGTGTTGTGAAATGT | |
| | TGGGTTAAGTCCCGCAACGAGCGCAACCCTTATCCTTTGTTGCCAGCGGTCCGGCCGGGAA | |
| | CTCAAAGGAGACTGCCAGTGATAAACTGGAGGAAGGTGGGGATGACGTCAAGTCATCATGG | |
| | CCCTTACGACCAGGGCTACACACGTGCTACAATGGCGCATACAAAGAGAAGCGACCTCGCG | |
| | AGAGCAAGCGGACCTCATAAAGTGCGTCGTAGTCCGGATTGGAGTCTGCAACTCGACTCCA | |
| | TGAAGTCGGAATCGCTAGTAATCGTGGATCAGAATGCCACGGTGAATACGTTCCCGGGCCT | |
| | TGTACACACCGCCCGTCACACCATGGGAGTGGGTTGCAAAAGAAGTAGGTAGCTTAACCTT | |
| | CGGGAGGGCGCTTACCACTTTGTGATTCATGACTGGGGTGAAGTCGTAACAAGGTAACCGT | |
| | AGGGGAACCTGCGGTTGGATCATGTATATACCTTAAAGAAGCGTACTTTGTAGTGCTCACA | |
| | CAGATTGTCTGATAGAAAGTGAAAAGCAAGGCGTTTACGCGTTGGGAGTGAGGCagaagga | |
| | gattttcaacatgctccctcaatcggttgaatgtcgcccttttgtctttggcgctggtaaa | |
| | ccatatgaattttctattgattgtgacaaaataaacttattccgtggtgtctttgcgtttc | |
| | ttttatatgttgccacctttatgtatgtatCttctacgtttgctaacatactgcgtaataa | |
| | ggagtcttaatcatgccagttcttttgggtattccgttattattgcgtttcctcggtttcc | |
| | ttctggtaactttgttcggctatctgcttacttttctCaaaaagggcttcggtaagatagc | |
| | tattgctatttcattgtttcttgctcttattattgggcttaactcaattcttgtgggttat | |
| | ctctctgatattagTgctcaattaccctctgactttgttcagggtgttcagttaattctcc | |
| | cgtctaatgcgcttccctgtttttatgttattctctctgtaaagActgctattttcatttt | |
| | tgacgttaaacaaaaaatcgtttcttatttggattgggaCaaataatatggctgtttattt | |
| | tgtaactggcaaattaggctctggaaagacgctcgttagcgttggtaagattcaggataaa | |
| | attgtagctgggtgcaaaatagcaactaatcttgatttaaggcttcaaaacctcccgcaag | |
| | tcgggaggttcgctaaaacgcctcgcgttcttagaataccggataagccttctatatctga | |
| | tttgcttgctattgggcgcggtaatgattcctacgatgaaaataaaaacggAttgcttgtt | |
| | ctcgatgagtgcggtacttggtttaatacccgttcttggaatgataaggaaagacagccga | |
| | ttattgattggtttctacatgctcgtaaattaggatgggatattatttttcttgttcagga | |
| | cttatctattgttgataaacaggcgcgttctgcattagctgaacatgttgtttattgtcgt | |
| | cgtctggacagaattactttaccttttgtcggtactttatattctcttattactggctcga | |
| | aaatgcctctgcctaaattacatgttggcgttgttaaatatggcgattctcaattaagccc | |
| | tactgttgagcgttggctttatactggtaagaatttgtataacgcataCgatactaaacag | |
| | gctttttctagtaattatgattccggtgtttattcttatttaacgccttatttatcacacg | |
| | gtcggtatttcaaaccattaaatttaggtcagaagatgaaattaactaaaatatatttgaa | |
| | aaagttttctcgcgttctttgtcttgcgattggatttgcatcagcatttacatatagttat | |
| | ataacccaacctaagccggaggttaaaaaggtagtctctcagacctatgattttgataaat | |
| | tcactattgactcttctcaTcgtcttaatctaagctatcgctatgttttcaaggattctaa | |
| | gggaaaattaattaatagcgacgatttacagaagcaaggttattcactTacatatattgat | |
| | ttatgtactgtttccattaaaaaaggtaattcaaatgaaattgttaaatgtaattaatttt | |
| | gttttcttgatgtttgtttcatcatcttcttttgctcaggtaattgaaatgaataattcgc | |
| | ctctgcgcgattttAtaacttggtattcaaagcaatcaggcgaatccgttattgtttctcc | |
| | cgatgtaaaaggtactgttactgtatattcatctgacgttaaacctgaaaatctacgcaat | |
| | ttctttatttctgttttacgtgcaaataattttgatatggtaggttctaacccttccatta | |
| | ttcagaagtataatccaaacaatcaggattatattgatgaattgccatcaCctgataatca | |
| | ggaatatgatgataattccgctccttctggtggtttctttgtCccgcaaaatgataatgtt | |
| | actcaaacttttaaaattaataacgttcgggcaaaggatttaatacgagttgtcgaattgt | |
| | ttgtaaagtctaatacttctaaatcctcaaatgtattatctattgacggAtctaatctatt | |
| | agttgttagtgctcctaaagatattttagataaccttTctcaattcctttcaactgttgat | |
| | ttgccaactgaccagGtattgattgagggtttgatatttgaggttcagcaaggtgatgctt | |
| | tagatttttcatttgctgctggctctcagcgtggcactgttgcaggcggAgttaatactga | |
| | ccgcctcacctctgttttatcttctgctggtggttcgttcggtatttttaatggcgatgtt | |
| | ttagggctatcagttcgcgcattaaagactaatagccattcaaaaatattgtctgtgccac | |
| | gtattcttacgctttcaggtcagaagggttctatctctAttggccagaatgtcccttttat | |
| | tactggtcgtgtAactggtgaatctgccaatgtaaataatccatttcagacgattgagcgt | |
| | caaaatgtaggtatttccatgagcgtttttcctgttgcaatggctggcggtaatattgttc | |
| | tggatattaccagcaaggccgatagtttgagttcttctactcaggcaagtgatgttattac | |
| | taaCcaaagaagtattgctacaacggttaatttgcgtgatggacagactcttCtactcggt | |
| | ggcctcactgattataaaaacacttctcaggattctggcgtaccgttcctgtctaaaatcc | |
| | ctttaatcggcctcctgtttagctcccgctctgattctaacgaggaGagcacgttatacgt | |
| | gctcgtcaaagcaaccatagtacgcgccctgtagcggcgcattaagcgcggcgggtgtggt | |
| | ggttacgcgcagcgtgaccgctacacttgccagcgccctagcgcccgctcctttcgctttc | |
| | ttcccttcctttctcgccacgttcgccggctttccccgtcaagcGctaaatcgggggcccc | |
| | tttagggttccgatttagtgctttacggcacctcgaccccaaaaaacttgatttgggtgat | |
| | ggttcacgtagtgggccatcgccctgatagacggtttttcgccctttgacgttggagtcca | |
| | cgttctttaatagtggactcttgttccaaactggaacaacactcaaccctatctcgggcta | |
| | ttcttttgatttataagggattttgccgatttcggcctattggttaaaaaatgaActgatt | |
| | taacaaaaatttaacgcgaattttaacaaaatattaacgtttacaatttaaatatttgctt | |
| | atacaatcttcctgtCttGggggcttttcttattatcaaccggggtacat | |
| SP_{H3-1} | atgattgacatgctagttttacgGttaccgttcatcgattctcttgtttgctccagactct | 98 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatctctcacctaccaaacaatgcccccctgcaaa | |
| | aaataaattcatataaaaaacatacagataaccatctgcggtgataaattatctctggcgg | |
| | tgttgacataaataccactggcggttatactgagcacgggtaccGGCCGCTGAGAAAAAGC | |
| | GAAGCGGCACTGCTCTTTAACAATTTATCAGACAATCTGTGTGGGCACTCGAAGATACGGA | |
| | TTCTTAACGTCGCAAGACGAAAAATGAATACCAAGTCTCAAGAGTGAACACGTAATTCATT | |
| | ACGAAGTTTAATTCTTTGAGCGTCAAACTTTTAAATTGAAGAGTTTGATCATGGCTCAGAT | |
| | TGAACGCTGGCGGCAGGCCTAACACATGCAAGTCGAACGGTAACAGGAAGAAGCTTGCTTC | |
| | TTTGCTGACGAGTGGCGGACGGGTGAGTAATGTCTGGGAAACTGCCTGATGGAGGGGGATA | |
| | ACTACTGGAAACGGTAGCTAATACCGCATAACGTCGCAAGACCAAAGAGGGGGACCTTCGG | |
| | GCCTCTTGCCATCGGATGTGCCCAGATGGGATTAGCTAGTAGGTGGGGTAACGGCTCACCT | |
| | AGGCGACGATCCCTAGCTGGTCTGAGAGGATGACCAGCCACACTGGAACTGAGACACGGTC | |
| | CAGACTCCTACGGGAGGCAGCAGTGGGGAATATTGCACAATGGGCGCAAGCCTGATGCAGC | |
| | CATGCCGCGTGTATGAAGAAGGCCTTCGGGTTGTAAAGTACTTTCAGCGGGGAGGAAGGGA | |
| | GTAAAGTTAATACCTTTGCTCATTGACGTTACCCGCAGAAGAAGCACCGGCTAACTCCGTG | |
| | CCAGCAGCCGCGGTAATACGGAGGGTGCAAGCGTTAATCGGAATTACTGGGCGTAAAGCGC | |
| | ACGCAGGCGGTTTGTTAAGTCAGATGTGAAATCCCCGGGCTCAACCTGGGAACTGCATCTG | |
| | ATACTGGCAAGCTTGAGTCTCGTAGAGGGGGGTAGAATTCCAGGTGTAGCGGTGAAATGCG | |
| | TAGAGATCTGGAGGAATACCGGTGGCGAAGGCGGCCCCCTGGACGAAGACTGACGCTCAGG | |
| | TGCGAAAGCGTGGGGAGCAAACAGGATTAGATACCCTGGTAGTCCACGCCGTAAACGATGT | |
| | CGACTTGGAGGTTGTGCCCTTGAGGCGTGGCTTCCGGAGCTAACGCGTTAAGTCGACCGCC | |
| | TGGGGAGTACGGCCGCAAGGTTAAAACTCAAATGAATTGACGGGGGCCCGCACAAGCGGTG | |
| | GAGCATGTGGTTTAATTCGATGCAACGCGAAGAACCTTACCTGGTCTTGACATCCACGGAA | |
| | GTTTTCAGAGATGAGAATGTGCCTTCGGGAACCGTGAGACAGGTGCTGCATGGCTGTCGTC | |
| | AGCTCGTGTTGTGAAATGTTGGGTTAAGTCCCGCAACGAGCGCAACCCTTATCCTTTGTTG | |
| | CCAGCGGTCCGGCCGGGAACTCAAAGGAGACTGCCAGTGATAAACTGGAGGAAGGTGGGGA | |
| | TGACGTCAAGTCATCATGGCCCTTACGACCAGGGCTACACACGTGCTACAATGGCGCATAC | |
| | AAAGAGAAGCGACCTCGCGAGAGCAAGCGGACCTCATAAAGTGCGTCGTAGTCCGGATTGG | |
| | AGTCTGCAACTCGACTCCATGAAGTCGGAATCGCTAGTAATCGTGGATCAGAATGCCACGG | |
| | TGAATACGTTCCCGGGCCTTGTACACACCGCCCGTCACACCATGGGAGTGGGTTGCAAAAG | |
| | AAGTAGGTAGCTTAACCTTCGGGAGGGCGCTTACCACTTTGTGATTCATGACTGGGGTGAA | |
| | GTCGTAACAAGGTAACCGTAGGGGAACCTGCGGTTGGATCACTTGTATACCTTAAAGAAGC | |
| | GTACTTTGTAGTGCTCACACAGATTGTCTGATAGAAAGTGAAAAGCAAGGCGTTTACGCGT | |
| | TGGGAGTGAGGCTGAAGAGAATAAGGCCGTTCGCTTTCTATTAATGAAAGCTCACCCTACA | |
| | CGAAAATATCACGCAACGCGTGATAAGCAATTTTCGTGTCCCCTTCGTCTAGAGGCCCAGG | |
| | ACACCGCCCTTTCACGGCGGTAACAGGGGTTCGAATCCCCTAGGGGACGCCACTTGCTGGT | |
| | TTGTGAGTGAAAGTCGCCGACCTTAATATCTCAAAACTCATCTTCGGGTGATGTTTGAGAT | |
| | ATTTGCTCTTTAAAAATCTGGATCAAGCTGAAAATTGAAACACTGAACAACGAGAGTTGTT | |
| | CGTGAGTCTCTCAAATTTTCGCAACACGATGATGAATCGAAAGAAACATCTTCGGGTTGTG | |
| | AGGTTAAGCGACTAAGCGTACACGGTGGATGCCCTGGCAGTCAGAGGCGATGAAGGACGTG | |
| | CTAATCTGCGATAAGCGTCGGTAAGGTGATATGAACCGTTATAACCGGCGATTTCCGAATG | |
| | GGGAAACCCAGTGTGTTTCGACACACTATCATTAACTGAATCCATAGGTTAATGAGGCGAA | |
| | CCGGGGGAACTGAAACATCTAAGTACCCCGAGGAAAAGAAATCAACCGAGATTCCCCCAGT | |
| | AGCGGCGAGCGAACGGGGAGCAGCCCAGAGCCTGAATCAGTGTGTGTGTTAGTGGAAGCGT | |
| | CTGGAAAGGCGCGCGATACAGGGTGACAGCCCCGTACACAAAAATGCACATGCTGTGAGCT | |
| | CGATGAGTAGGGCGGGACACGTGGTATCCTGTCTGAATATGGGGGGACCATCCTCCAAGGC | |
| | TAAATACTCCTGACTGACCGATAGTGAACCAGTACCGTGAGGGAAAGGCGAAAAGAACCCC | |
| | GGCGAGGGGAGTGAAAAAGAACCTGAAACCGTGTACGTACAAGCAGTGGGAGCACGCTTAG | |
| | GCGTGTGACTGCGTACCTTTTGTATAATGGGTCAGCGACTTATATTCTGTAGCAAGGTTAA | |
| | CCGAATAGGGGAGCCGAAGGGAAACCGAGTCTTAACTGGGCGTTAAGTTGCAGGGTATAGA | |
| | CCCGAAACCCGGTGATCTAGCCATGGGCAGGTTGAAGGTTGGGTAACACTAACTGGAGGAC | |
| | CGAACCGACTAATGTTGAAAAATTAGCGGATGACTTGTGGCTGGGGGTGAAAGGCCAATCA | |
| | AACCGGGAGATAGCTGGTTCTCCCCGAAAGCTATTTAGGTAGCGCCTCGTGAATTCATCTC | |
| | CGGGGGTAGAGCACTGTTTCGGCAAGGGGGTCATCCCGACTTACCAACCCGATGCAAACTG | |
| | CGAATACCGGAGAATGTTATCACGGGAGACACACGGCGGGTGCTAACGTCCGTCGTGAAGA | |
| | GGGAAACAACCCAGACCGCCAGCTAAGGTCCCAAAGTCATGGTTAAGTGGGAAACGATGTG | |
| | GGAAGGCCCAGACAGCCAGGATGTTGGCTTAGAAGCAGCCATCATTTAAAGAAAGCGTAAT | |
| | AGCTCACTGGTCGAGTCGGCCTGCGCGGAAGATGTAACGGGGCTAAACCATGCACCGAAGC | |
| | TGCGGCAGCGACGCTTATGCGTTGTTGGGTAGGGGAGCGTTCTGTAAGCCTGCGAAGGTGT | |
| | GCTGTGAGGCATGCTGGAGGTATCAGAAGTGCGAATGCTGACATAAGTAACGATAAAGCGG | |
| | GTGAAAAGCCCGCTCGCCGGAAGACCAAGGGTTCCTGTCCAACGTTAATCGGGGCAGGGTG | |
| | AGTCGACCCCTAAGGCGAGGCCGAAAGGCGTAGTCGATGGGAAACAGGTTAATATTCCTGT | |
| | ACTTGGTGTTACTGCGAAGGGGGGACGGAGAAGGCTATGTTGGCCGGGCGACGGTTGTCCC | |
| | GGTTTAAGCGTGTAGGCTGGTTTTCCAGGCAAATCCGGAAAATCAAGGCTGAGGCGTGATG | |
| | ACGAGGCACTACGGTGCTGAAGCAACAAATGCCCTGCTTCCAGGAAAAGCCTCTAAGCATC | |
| | AGGTAACATCAAATCGTACCCCAAACCGACACAGGTGGTCAGGTAGAGAATACCAAGGCGC | |
| | TTGAGAGAACTCGGGTGAAGGAACTAGGCAAAATGGTGCCGTAACTTCGGGAGAAGGCACG | |
| | CTGATATGTAGGTGAGGTCCCTCGCGGATGGAGCTGAAATCAGTCGAAGATACCAGCTGGC | |
| | TGCAACTGTTTATTAAAAACACAGCACTGTGCAAACACGAAAGTGGACGTATACGGTGTGA | |
| | CGCCTGCCCGGTGCCGGAAGGTTAATTGATGGGGTTAGCGCAAGCGAAGCTCTTGATCGAA | |
| | GCCCCGGTAAACGGCGGCCGTAACTATAACGGTCCTAAGGTAGCGAAATTCCTTGTCGGGT | |
| | AAGTTCCGACCTGCACGAATGGCGTAATGATGGCCAGGCTGTCTCCACCCGAGACTCAGTG | |
| | AAATTGAACTCGCTGTGAAGATGCAGTGTACCCGCGGCAAGACGGAAAGACCCCGTGAACC | |
| | TTTACTATAGCTTGACACTGAACATTGAGCCTTGATGTGTAGGATAGGTGGGAGGCTTTGA | |
| | AGTGTGGACGCCAGTCTGCATGGAGCCGACCTTGAAATACCACCCTTTAATGTTTGATGTT | |
| | CTAACGTTGACCCGTAATCCGGGTTGCGGACAGTGTCTGGTGGGTAGTTTGACTGGGGCGG | |
| | TCTCCTCCTAAAGAGTAACGGAGGAGCACGAAGGTTGGCTAATCCTGGTCGGACATCAGGA | |
| | GGTTAGTGCAATGGCATAAGCCAGCTTGACTGCGAGCGTGACGGCGCGAGCAGGTGCGAAA | |
| | GCAGGTCATAGTGATCCGGTGGTTCTGAATGGAAGGGCCATCGCTCAACGGATAAAAGGTA | |
| | CTCCGGGGATAACAGGCTGATACCGCCCAAGAGTTCATATCGACGGCGGTGTTTGGCACCT | |
| | CGATGTCGGCTCATCACATCCTGGGGCTGAAGTAGGTCCCAAGGGTATGGCTGTTCGCCAT | |
| | TTAAAGTGGTACGCGAGCTGGGTTTAGAACGTCGTGAGACAGTTCGGTCCCTATCTGCCGT | |
| | GGGCGCTGGAGAACTGAGGGGGGCTGCTCCTAGTACGAGAGGACCGGAGTGGACGCATCAC | |
| | TGGTGTTCGGGTTGTCATGCCAATGGCACTGCCCGGTAGCTAAATGCGGAAGAGATAAGTG | |
| | CTGAAAGCATCTAAGCACGAAACTTGCCCCGAGATGAGTTCTCCCTGACCCTTTAAGGGTC | |
| | CTGAAGGAACGTTGAAGACGACGACGTTGATAGGCCGGGTGTGTAAGCGCAGCGATGCGTT | |
| | GAGCTAACCGGTACTAATGAACCGTGAGGCTTAACCTTACAACGCCGAAGCTGTTTTGGCG | |
| | GATGAGAGAAGATTTTCAGCCTGATACAGATTAAATCAGAACGCAGAAGCGGTCTGATAAA | |
| | ACAGAATTTGCCTGGCGGCAGTAGCGCGGTGGTCCCACCTGACCCCATGCCGAACTCAGAA | |
| | GTGAAACGCCGTAGCGCCGATGGTAGTGTGGGGTCTCCCCATGCGAGAGTAGGGAACTGCC | |
| | AGGCATCAAATAAAACGAAAGGCTCAGTCGAAAGACTGGGCCTTTCGTTTTATCTGTTGTT | |
| | TGTCGGTGAACGCTCTCCagaaggagattttcaacatgctccctcaatcggttgaatgtcg | |
| | cccttttgtctttggcgctggtaaaccatatgaattttctattgattgtgacaaaataaac | |
| | ttattccgtggtgtctttgcgtttcttttatatgttgccacctttatgtatgtatCttcta | |
| | cgtttgctaacatactgcgtaataaggagtcttaatcatgccagttcttttgggtattccg | |
| | ttattattgcgtttcctcggtttccttctggtaactttgttcggctatctgcttacttttc | |
| | tCaaaaagggcttcggtaagatagctattgctatttcattgtttcttgctcttattattgg | |
| | gcttaactcaattcttgtgggttatctctctgatattagTgctcaattaccctctgacttt | |
| | gttcagggtgttcagttaattctcccgtctaatgcgcttccctgtttttatgttattctct | |
| | ctgtaaagActgctattttcatttttgacgttaaacaaaaaatcgtttcttatttggattg | |
| | ggaCaaataatatggctgtttattttgtaactggcaaattaggctctggaaagacgctcgt | |
| | tagcgttggtaagattcaggataaaattgtagctgggtgcaaaatagcaactaatcttgat | |
| | ttaaggcttcaaaacctcccgcaagtcgggaggttcgctaaaacgcctcgcgttcttagaa | |
| | taccggataagccttctatatctgatttgcttgctattgggcgcggtaatgattcctacga | |
| | tgaaaataaaaacggAttgcttgttctcgatgagtgcggtacttggtttaatacccgttct | |
| | tggaatgataaggaaagacagccgattattgattggtttctacatgctcgtaaattaggat | |
| | gggatattatttttcttgttcaggacttatctattgttgataaacaggcgcgttctgcatt | |
| | agctgaacatgttgtttattgtcgtcgtctggacagaattactttaccttttgtcggtact | |
| | ttatattctcttattactggctcgaaaatgcctctgcctaaattacatgttggcgttgtta | |
| | aatatggcgattctcaattaagccctactgttgagcgttggctttatactggtaagaattt | |
| | gtataacgcataCgatactaaacaggctttttctagtaattatgattccggtgtttattct | |
| | tatttaacgccttatttatcacacggtcggtatttcaaaccattaaatttaggtcagaaga | |
| | tgaaattaactaaaatatatttgaaaaagttttctcgcgttctttgtcttgcgattggatt | |
| | tgcatcagcatttacatatagttatataacccaacctaagccggaggttaaaaaggtagtc | |
| | tctcagacctatgattttgataaattcactattgactcttctcaTcgtcttaatctaagct | |
| | atcgctatgttttcaaggattctaagggaaaattaattaatagcgacgatttacagaagca | |
| | aggttattcactTacatatattgatttatgtactgtttccattaaaaaaggtaattcaaat | |
| | gaaattgttaaatgtaattaattttgttttcttgatgtttgtttcatcatcttcttttgct | |
| | caggtaattgaaatgaataattcgcctctgcgcgattttAtaacttggtattcaaagcaat | |
| | caggcgaatccgttattgtttctcccgatgtaaaaggtactgttactgtatattcatctga | |
| | cgttaaacctgaaaatctacgcaatttctttatttctgttttacgtgcaaataattttgat | |
| | atggtaggttctaacccttccattattcagaagtataatccaaacaatcaggattatattg | |
| | atgaattgccatcaCctgataatcaggaatatgatgataattccgctccttctggtggttt | |
| | ctttgtCccgcaaaatgataatgttactcaaacttttaaaattaataacgttcgggcaaag | |
| | gatttaatacgagttgtcgaattgtttgtaaagtctaatacttctaaatcctcaaatgtat | |
| | tatctattgacggAtctaatctattagttgttagtgctcctaaagatattttagataacct | |
| | tTctcaattcctttcaactgttgatttgccaactgaccagGtattgattgagggtttgata | |
| | tttgaggttcagcaaggtgatgctttagatttttcatttgctgctggctctcagcgtggca | |
| | ctgttgcaggcggAgttaatactgaccgcctcacctctgttttatcttctgctggtggttc | |
| | gttcggtatttttaatggcgatgttttagggctatcagttcgcgcattaaagactaatagc | |
| | cattcaaaaatattgtctgtgccacgtattcttacgctttcaggtcagaagggttctatct | |
| | ctAttggccagaatgtcccttttattactggtcgtgtAactggtgaatctgccaatgtaaa | |
| | taatccatttcagacgattgagcgtcaaaatgtaggtatttccatgagcgtttttcctgtt | |
| | gcaatggctggcggtaatattgttctggatattaccagcaaggccgatagtttgagttctt | |
| | ctactcaggcaagtgatgttattactaaCcaaagaagtattgctacaacggttaatttgcg | |
| | tgatggacagactcttCtactcggtggcctcactgattataaaaacacttctcaggattct | |
| | ggcgtaccgttcctgtctaaaatccctttaatcggcctcctgtttagctcccgctctgatt | |
| | ctaacgaggaGagcacgttatacgtgctcgtcaaagcaaccatagtacgcgccctgtagcg | |
| | gcgcattaagcgcggcgggtgtggtggttacgcgcagcgtgaccgctacacttgccagcgc | |
| | cctagcgcccgctcctttcgctttcttcccttcctttctcgccacgttcgccggctttccc | |
| | cgtcaagcGctaaatcgggggcccctttagggttccgatttagtgctttacggcacctcga | |
| | ccccaaaaaacttgatttgggtgatggttcacgtagtgggccatcgccctgatagacggtt | |
| | tttcgccctttgacgttggagtccacgttctttaatagtggactcttgttccaaactggaa | |
| | caacactcaaccctatctcgggctattcttttgatttataagggattttgccgatttcggc | |
| | ctattggttaaaaaatgaActgatttaacaaaaatttaacgcgaattttaacaaaatatta | |
| | acgtttacaatttaaatatttgcttatacaatcttcctgtCttGggggcttttcttattat | |
| | caaccggggtacat | |
| SP_{lib} | atgattgacatgctagttttacgGttaccgttcatcgattctcttgtttgctccagactct | 99 |
| | caggcaatgacctgatagcctttgtagaTctctcaaaaatagctaccctctccggcatgaa | |
| | tttatcagctagaacggttgaatatcatgttgatggtgatttgactgtctccggcctttcC | |
| | cacccttttgaatctttacctacacattactcaggcattgcatttaaaatatatgagggtt | |
| | ctaaaaatttttatccttgcgttgaaataaaggcttctcccgcaaaagtattacagggGca | |
| | taatgtttttggtacaaccgatttagGtttatgctctgaggctttattgcttaattttgct | |
| | aattctttgccttgcctgtatgatttattggatgttaacgctactactattagtagaattg | |
| | atgccGccttttcagcCcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | AcaattaaGgAtactctctaatcctgacctgttggagtttgcttccggtctggttcgcttt | |
| | gaAgctcgaattaaaacgAgatatttgaagtctttcgggcttcctcttaatctttttgatA | |
| | caatccgctttgcttctgactataatagtcagggtaaagacctgatttttgatttatggtc | |
| | attctcgttttctgaactgtttaaagcatttgaggggAattcaatgaatatttatgacgat | |
| | tccgcagtattggCcgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagTtttg | |
| | ttacgttgatttgggtaatgaatatccggttcttgtcaagattactcttgatgaaggtcag | |
| | ccagcctatgcgccGggtctgtacaccgttcatctgtcctctttcaaagttggtcagttcg | |
| | gttcccttatgattgaccgtctgcgcctcgttccggctaagtaacatggagcaggtcgcgg | |
| | atttcgacaTaatttatcaggcgatgatacaaatctccgttgtactCtgtttcgcgcttgg | |
| | tataatAgctgggggtcaaagatgagtgttttagtgtattctttcgcctctttcgttttag | |
| | gttggtgcctCcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttGactccctgcaagcctcagcgaccga | |
| | atatatcggttatgcgtgggcgatggttgttgtcattgtcggcgcaactatcggtatcaag | |
| | ctgtttaagaaattcacctcgaaagcaagTtgataaacTgatacaattaaaggctcctttt | |
| | ggagcctttttttttgatgcggccgcgatGCTGAGAAAAAGCGAAGCGGCACTGCTCTTTA | |
| | ACAATTTATCAGACAATCTGTGTGGGCACTCGAAGATACGGATTCTTAACGTCGCAAGACG | |
| | AAAAATGAATACCAAGTCTCAAGAGTGAACACGTAATTCATTACGAAGTTTAATTCTTTGA | |
| | GCGTCAAACTTTTAAATTGAAGAGTTTGATCATGGCTCAGATTGAACGCTGGCGGCAGGCC | |
| | TAACACATGCAAGTCGAACGGTAACAGGAAGAAGCTTGCTTCTTTGCTGACGAGTGGCGGA | |
| | CGGGTGAGTAATGTCTGGGAAACTGCCTGATGGAGGGGGATAACTACTGGAAACGGTAGCT | |
| | AATACCGCATAACGTCGCAAGACCAAAGAGGGGGACCTTCGGGCCTCTTGCCATCGGATGT | |
| | GCCCAGATGGGATTAGCTAGTAGGTGGGGTAACGGCTCACCTAGGCGACGATCCCTAGCTG | |
| | GTCTGAGAGGATGACCAGCCACACTGGAACTGAGACACGGTCCAGACTCCTACGGGAGGCA | |
| | GCAGTGGGGAATATTGCACAATGGGCGCAAGCCTGATGCAGCCATGCCGCGTGTATGAAGA | |
| | AGGCCTTCGGGTTGTAAAGTACTTTCAGCGGGGAGGAAGGGAGTAAAGTTAATACCTTTGC | |
| | TCATTGACGTTACCCGCAGAAGAAGCACCGGCTAACTCCGTGCCAGCAGCCGCGGTAATAC | |
| | GGAGGGTGCAAGCGTTAATCGGAATTACTGGGCGTAAAGCGCACGCAGGCGGTTTGTTAAG | |
| | TCAGATGTGAAATCCCCGGGCTCAACCTGGGAACTGCATCTGATACTGGCAAGCTTGAGTC | |
| | TCGTAGAGGGGGGTAGAATTCCAGGTGTAGCGGTGAAATGCGTAGAGATCTGGAGGAATAC | |
| | CGGTGGCGAAGGCGGCCCCCTGGACGAAGACTGACGCTCAGGTGCGAAAGCGTGGGGAGCA | |
| | AACAGGATTAGATACCCTGGTAGTCCACGCCGTAAACGATGTCGACTTGGAGGTTGTGCCC | |
| | TTGAGGCGTGGCTTCCGGAGCTAACGCGTTAAGTCGACCGCCTGGGGAGTACGGCCGCAAG | |
| | GTTAAAACTCAAATGAATTGACGGGGGCCCGCACAAGCGGTGGAGCATGTGGTTTAATTCG | |
| | ATGCAACGCGAAGAACCTTACCTGGTCTTGACATCCACGGAAGTTTTCAGAGATGAGAATG | |
| | TGCCTTCGGGAACCGTGAGACAGGTGCTGCATGGCTGTCGTCAGCTCGTGTTGTGAAATGT | |
| | TGGGTTAAGTCCCGCAACGAGCGCAACCCTTATCCTTTGTTGCCAGCGGTCCGGCCGGGAA | |
| | CTCAAAGGAGACTGCCAGTGATAAACTGGAGGAAGGTGGGGATGACGTCAAGTCATCATGG | |
| | CCCTTACGACCAGGGCTACACACGTGCTACAATGGCGCATACAAAGAGAAGCGACCTCGCG | |
| | AGAGCAAGCGGACCTCATAAAGTGCGTCGTAGTCCGGATTGGAGTCTGCAACTCGACTCCA | |
| | TGAAGTCGGAATCGCTAGTAATCGTGGATCAGAATGCCACGGTGAATACGTTCCCGGGCCT | |
| | TGTACACACCGCCCGTCACACCATGGGAGTGGGTTGCAAAAGAAGTAGGTAGCTTAACCTT | |
| | CGGGAGGGCGCTTACCACTTTGTGATTCATGACTGGGGTGAAGTCGTAACAAGGTAACCGT | |
| | AGGGGAACCTGCGGTTGGATCAnnnnnnTACCTTAAAGAAGCGTACTTTGTAGTGCTCACA | |
| | CAGATTGTCTGATAGAAAGTGAAAAGCAAGGCGTTTACGCGTTGGGAGTGAGGCagaagga | |
| | gattttcaacatgctccctcaatcggttgaatgtcgcccttttgtctttggcgctggtaaa | |
| | ccatatgaattttctattgattgtgacaaaataaacttattccgtggtgtctttgcgtttc | |
| | ttttatatgttgccacctttatgtatgtatCttctacgtttgctaacatactgcgtaataa | |
| | ggagtcttaatcatgccagttcttttgggtattccgttattattgcgtttcctcggtttcc | |
| | ttctggtaactttgttcggctatctgcttacttttctCaaaaagggcttcggtaagatagc | |
| | tattgctatttcattgtttcttgctcttattattgggcttaactcaattcttgtgggttat | |
| | ctctctgatattagTgctcaattaccctctgactttgttcagggtgttcagttaattctcc | |
| | cgtctaatgcgcttccctgtttttatgttattctctctgtaaagActgctattttcatttt | |
| | tgacgttaaacaaaaaatcgtttcttatttggattgggaCaaataatatggctgtttattt | |
| | tgtaactggcaaattaggctctggaaagacgctcgttagcgttggtaagattcaggataaa | |
| | attgtagctgggtgcaaaatagcaactaatcttgatttaaggcttcaaaacctcccgcaag | |
| | tcgggaggttcgctaaaacgcctcgcgttcttagaataccggataagccttctatatctga | |
| | tttgcttgctattgggcgcggtaatgattcctacgatgaaaataaaaacggAttgcttgtt | |
| | ctcgatgagtgcggtacttggtttaatacccgttcttggaatgataaggaaagacagccga | |
| | ttattgattggtttctacatgctcgtaaattaggatgggatattatttttcttgttcagga | |
| | cttatctattgttgataaacaggcgcgttctgcattagctgaacatgttgtttattgtcgt | |
| | cgtctggacagaattactttaccttttgtcggtactttatattctcttattactggctcga | |
| | aaatgcctctgcctaaattacatgttggcgttgttaaatatggcgattctcaattaagccc | |
| | tactgttgagcgttggctttatactggtaagaatttgtataacgcataCgatactaaacag | |
| | gctttttctagtaattatgattccggtgtttattcttatttaacgccttatttatcacacg | |
| | gtcggtatttcaaaccattaaatttaggtcagaagatgaaattaactaaaatatatttgaa | |
| | aaagttttctcgcgttctttgtcttgcgattggatttgcatcagcatttacatatagttat | |
| | ataacccaacctaagccggaggttaaaaaggtagtctctcagacctatgattttgataaat | |
| | tcactattgactcttctcaTcgtcttaatctaagctatcgctatgttttcaaggattctaa | |
| | gggaaaattaattaatagcgacgatttacagaagcaaggttattcactTacatatattgat | |
| | ttatgtactgtttccattaaaaaaggtaattcaaatgaaattgttaaatgtaattaatttt | |
| | gttttcttgatgtttgtttcatcatcttcttttgctcaggtaattgaaatgaataattcgc | |
| | ctctgcgcgattttAtaacttggtattcaaagcaatcaggcgaatccgttattgtttctcc | |
| | cgatgtaaaaggtactgttactgtatattcatctgacgttaaacctgaaaatctacgcaat | |
| | ttctttatttctgttttacgtgcaaataattttgatatggtaggttctaacccttccatta | |
| | ttcagaagtataatccaaacaatcaggattatattgatgaattgccatcaCctgataatca | |
| | ggaatatgatgataattccgctccttctggtggtttctttgtCccgcaaaatgataatgtt | |
| | actcaaacttttaaaattaataacgttcgggcaaaggatttaatacgagttgtcgaattgt | |
| | ttgtaaagtctaatacttctaaatcctcaaatgtattatctattgacggAtctaatctatt | |
| | agttgttagtgctcctaaagatattttagataaccttTctcaattcctttcaactgttgat | |
| | ttgccaactgaccagGtattgattgagggtttgatatttgaggttcagcaaggtgatgctt | |
| | tagatttttcatttgctgctggctctcagcgtggcactgttgcaggcggAgttaatactga | |
| | ccgcctcacctctgttttatcttctgctggtggttcgttcggtatttttaatggcgatgtt | |
| | ttagggctatcagttcgcgcattaaagactaatagccattcaaaaatattgtctgtgccac | |
| | gtattcttacgctttcaggtcagaagggttctatctctAttggccagaatgtcccttttat | |
| | tactggtcgtgtAactggtgaatctgccaatgtaaataatccatttcagacgattgagcgt | |
| | caaaatgtaggtatttccatgagcgtttttcctgttgcaatggctggcggtaatattgttc | |
| | tggatattaccagcaaggccgatagtttgagttcttctactcaggcaagtgatgttattac | |
| | taaCcaaagaagtattgctacaacggttaatttgcgtgatggacagactcttCtactcggt | |
| | ggcctcactgattataaaaacacttctcaggattctggcgtaccgttcctgtctaaaatcc | |
| | ctttaatcggcctcctgtttagctcccgctctgattctaacgaggaGagcacgttatacgt | |
| | gctcgtcaaagcaaccatagtacgcgccctgtagcggcgcattaagcgcggcgggtgtggt | |
| | ggttacgcgcagcgtgaccgctacacttgccagcgccctagcgcccgctcctttcgctttc | |
| | ttcccttcctttctcgccacgttcgccggctttccccgtcaagcGctaaatcgggggcccc | |
| | tttagggttccgatttagtgctttacggcacctcgaccccaaaaaacttgatttgggtgat | |
| | ggttcacgtagtgggccatcgccctgatagacggtttttcgccctttgacgttggagtcca | |
| | cgttctttaatagtggactcttgttccaaactggaacaacactcaaccctatctcgggcta | |
| | ttcttttgatttataagggattttgccgatttcggcctattggttaaaaaatgaActgatt | |
| | taacaaaaatttaacgcgaattttaacaaaatattaacgtttacaatttaaatatttgctt | |
| | atacaatcttcctgtCttGggggcttttcttattatcaaccggggtacat | |
| HP | atgattgacatgctagttttacgattaccgttcatcgattctcttgtttgctccagactct | 100 |
| | caggcaatgacctgatagcctttgtagacctctcaaaaatagctaccctctccggcatgaa | |
| | tttatcagctagaacggttgaatatcatgttgatggtgatttgactgtctccggcctttct | |
| | cacccttttgaatctttacctacacattactcaggcattgcatttaaaatatatgagggtt | |
| | ctaaaaatttttatccttgcgttgaaataaaggcttctcccgcaaaagtattacagggtca | |
| | taatgtttttggtacaaccgatttagctttatgctctgaggctttattgcttaattttgct | |
| | aattctttgccttgcctgtatgatttattggatgttaacgctactactattagtagaattg | |
| | atgccaccttttcagctcgcgccccaaatgaaaatatagctaaacaggttattgaccattt | |
| | gcgaaatgtatctaatggtcaaactaaatctactcgttcgcagaattgggaatcaactgtt | |
| | acatggaatgaaacttccagacaccgtactttagttgcatatttaaaacatgttgagctac | |
| | agcaccagattcagcaattaagctctaagccatccgcaaaaatgacctcttatcaaaagga | |
| | gcaattaaaggtactctctaatcctgacctgttggagtttgcttccggtctggttcgcttt | |
| | gaagctcgaattaaaacgcgatatttgaagtctttcgggcttcctcttaatctttttgatg | |
| | caatccgctttgcttctgactataatagtcagggtaaagacctgatttttgatttatggtc | |
| | attctcgttttctgaactgtttaaagcatttgagggggattcaatgaatatttatgacgat | |
| | tccgcagtattggacgctatccagtctaaacattttactattaccccctctggcaaaactt | |
| | cttttgcaaaagcctctcgctattttggtttttatcgtcgtctggtaaacgagggttatga | |
| | tagtgttgctcttactatgcctcgtaattccttttggcgttatgtatctgcattagttgaa | |
| | tgtggtattcctaaatctcaactgatgaatctttctacctgtaataatgttgttccgttag | |
| | ttcgttttattaacgtagatttttcttcccaacgtcctgactggtataatgagccagttct | |
| | taaaatcgcataaggtaattcacaatgattaaagttgaaattaaaccatctcaagcccaat | |
| | ttactactcgttctggtgtttctcgtcagggcaagccttattcactgaatgagcagctttg | |
| | ttacgttgatttgggtaatgaatatccggttcttgtcaagattactcttgatgaaggtcag | |
| | ccagcctatgcgcctggtctgtacaccgttcatctgtcctctttcaaagttggtcagttcg | |
| | gttcccttatgattgaccgtctgcgcctcgttccggctaagtaacatggagcaggtcgcgg | |
| | atttcgacacaatttatcaggcgatgatacaaatctccgttgtactttgtttcgcgcttgg | |
| | tataatcgctgggggtcaaagatgagtgttttagtgtattctttcgcctctttcgttttag | |
| | gttggtgccttcgtagtggcattacgtattttacccgtttaatggaaacttcctcatgaaa | |
| | aagtctttagtcctcaaagcctctgtagccgttgctaccctcgttccgatgctgtctttcg | |
| | ctgctgagggtgacgatcccgcaaaagcggcctttaactccctgcaagcctcagcgaccga | |
| | atatatcggttatgcgtgggcgatggttgttgtcattgtcggcgcaactatcggtatcaag | |
| | ctgtttaagaaattcacctcgaaagcaagctgataaaccgatacaattaaaggctcctttt | |
| | ggagcctttttttttggagattttcaacgtgaaaaaattattattcgcaattcctttagtt | |
| | gttcctttctattctcactccgctgaaactgttgaaagttgtttagcaaaaccccatacag | |
| | aaaattcatttactaacgtctggaaagacgacaaaactttagatcgttacgctaactatga | |
| | gggctgtctgtggaatgctacaggcgttgtagtttgtactggtgacgaaactcagtgttac | |
| | ggtacatgggttcctattgggcttgctatccctgaaaatgagggtggtggctctgagggtg | |
| | gcggttctgagggtggcggttctgagggtggcggtactaaacctcctgagtacggtgatac | |
| | acctattccgggctatacttatatcaaccctctcgacggcacttatccgcctggtactgag | |
| | caaaaccccgctaatcctaatccttctcttgaggagtctcagcctcttaatactttcatgt | |
| | ttcagaataataggttccgaaataggcagggggcattaactgtttatacgggcactgttac | |
| | tcaaggcactgaccccgttaaaacttattaccagtacactcctgtatcatcaaaagccatg | |
| | tatgacgcttactggaacggtaaattcagagactgcgctttccattctggctttaatgagg | |
| | atccattcgtttgtgaatatcaaggccaatcgtctgacctgcctcaacctcctgtcaatgc | |
| | tggcggcggctctggtggtggttctggtggcggctctgagggtggtggctctgagggtggc | |
| | ggttctgagggtggcggctctgagggaggcggttccggtggtggctctggttccggtgatt | |
| | ttgattatgaaaagatggcaaacgctaataagggggctatgaccgaaaatgccgatgaaaa | |
| | cgcgctacagtctgacgctaaaggcaaacttgattctgtcgctactgattacggtgctgct | |
| | atcgatggtttcattggtgacgtttccggccttgctaatggtaatggtgctactggtgatt | |
| | ttgctggctctaattcccaaatggctcaagtcggtgacggtgataattcacctttaatgaa | |
| | taatttccgtcaatatttaccttccctccctcaatcggttgaatgtcgcccttttgtcttt | |
| | ggcgctggtaaaccatatgaattttctattgattgtgacaaaataaacttattccgtggtg | |
| | tctttgcgtttcttttatatgttgccacctttatgtatgtattttctacgtttgctaacat | |
| | actgcgtaataaggagtcttaatcatgccagttcttttgggtattccgttattattgcgtt | |
| | tcctcggtttccttctggtaactttgttcggctatctgcttacttttcttaaaaagggctt | |
| | cggtaagatagctattgctatttcattgtttcttgctcttattattgggcttaactcaatt | |
| | cttgtgggttatctctctgatattagcgctcaattaccctctgactttgttcagggtgttc | |
| | agttaattctcccgtctaatgcgcttccctgtttttatgttattctctctgtaaaggctgc | |
| | tattttcatttttgacgttaaacaaaaaatcgtttcttatttggattgggataaataatat | |
| | ggctgtttattttgtaactggcaaattaggctctggaaagacgctcgttagcgttggtaag | |
| | attcaggataaaattgtagctgggtgcaaaatagcaactaatcttgatttaaggcttcaaa | |
| | acctcccgcaagtcgggaggttcgctaaaacgcctcgcgttcttagaataccggataagcc | |
| | ttctatatctgatttgcttgctattgggcgcggtaatgattcctacgatgaaaataaaaac | |
| | ggcttgcttgttctcgatgagtgcggtacttggtttaatacccgttcttggaatgataagg | |
| | aaagacagccgattattgattggtttctacatgctcgtaaattaggatgggatattatttt | |
| | tcttgttcaggacttatctattgttgataaacaggcgcgttctgcattagctgaacatgtt | |
| | gtttattgtcgtcgtctggacagaattactttaccttttgtcggtactttatattctctta | |
| | ttactggctcgaaaatgcctctgcctaaattacatgttggcgttgttaaatatggcgattc | |
| | tcaattaagccctactgttgagcgttggctttatactggtaagaatttgtataacgcatat | |
| | gatactaaacaggctttttctagtaattatgattccggtgtttattcttatttaacgcctt | |
| | atttatcacacggtcggtatttcaaaccattaaatttaggtcagaagatgaaattaactaa | |
| | aatatatttgaaaaagttttctcgcgttctttgtcttgcgattggatttgcatcagcattt | |
| | acatatagttatataacccaacctaagccggaggttaaaaaggtagtctctcagacctatg | |
| | attttgataaattcactattgactcttctcagcgtcttaatctaagctatcgctatgtttt | |
| | caaggattctaagggaaaattaattaatagcgacgatttacagaagcaaggttattcactc | |
| | acatatattgatttatgtactgtttccattaaaaaaggtaattcaaatgaaattgttaaat | |
| | gtaattaattttgttttcttgatgtttgtttcatcatcttcttttgctcaggtaattgaaa | |
| | tgaataattcgcctctgcgcgattttgtaacttggtattcaaagcaatcaggcgaatccgt | |
| | tattgtttctcccgatgtaaaaggtactgttactgtatattcatctgacgttaaacctgaa | |
| | aatctacgcaatttctttatttctgttttacgtgcaaataattttgatatggtaggttcta | |
| | acccttccattattcagaagtataatccaaacaatcaggattatattgatgaattgccatc | |
| | atctgataatcaggaatatgatgataattccgctccttctggtggtttctttgttccgcaa | |
| | aatgataatgttactcaaacttttaaaattaataacgttcgggcaaaggatttaatacgag | |
| | ttgtcgaattgtttgtaaagtctaatacttctaaatcctcaaatgtattatctattgacgg | |
| | ctctaatctattagttgttagtgctcctaaagatattttagataaccttcctcaattcctt | |
| | tcaactgttgatttgccaactgaccagatattgattgagggtttgatatttgaggttcagc | |
| | aaggtgatgctttagatttttcatttgctgctggctctcagcgtggcactgttgcaggcgg | |
| | tgttaatactgaccgcctcacctctgttttatcttctgctggtggttcgttcggtattttt | |
| | aatggcgatgttttagggctatcagttcgcgcattaaagactaatagccattcaaaaatat | |
| | tgtctgtgccacgtattcttacgctttcaggtcagaagggttctatctctgttggccagaa | |
| | tgtcccttttattactggtcgtgtgactggtgaatctgccaatgtaaataatccatttcag | |
| | acgattgagcgtcaaaatgtaggtatttccatgagcgtttttcctgttgcaatggctggcg | |
| | gtaatattgttctggatattaccagcaaggccgatagtttgagttcttctactcaggcaag | |
| | tgatgttattactaatcaaagaagtattgctacaacggttaatttgcgtgatggacagact | |
| | cttttactcggtggcctcactgattataaaaacacttctcaggattctggcgtaccgttcc | |
| | tgtctaaaatccctttaatcggcctcctgtttagctcccgctctgattctaacgaggaaag | |
| | cacgttatacgtgctcgtcaaagcaaccatagtacgcgccctgtagcggcgcattaagcgc | |
| | tagcagtgagcttcatgtggcaggagaaaaaaggctgcaccggtgcgtcagcagaatatgt | |
| | gatacaggatatattccgcttcctcgctcactgactcgctacgctcggtcgttcgactgcg | |
| | gcgagcggaaatggcttacgaacggggcggagatttcctggaagatgccaggaagatactt | |
| | aacagggaagtgagagggccgcggcaaagccgtttttccataggctccgcccccctgacaa | |
| | gcatcacgaaatctgacgctcaaatcagtggtggcgaaacccgacaggactataaagatac | |
| | caggcgtttccccctggcggctccctcgtgcgctctcctgttcctgcctttcggtttaccg | |
| | gtgtcattccgctgttatggccgcgtttgtctcattccacgcctgacactcagttccgggt | |
| | aggcagttcgctccaagctggactgtatgcacgaaccccccgttcagtccgaccgctgcgc | |
| | cttatccggtaactatcgtcttgagtccaacccggaaagacatgcaaaagcaccactggca | |
| | gcagccactggtaattgatttagaggagttagtcttgaagtcatgcgccggttaaggctaa | |
| | actgaaaggacaagttttggtgactgcgctcctccaagccagttacctcggttcaaagagt | |
| | tggtagctcagagaaccttcgaaaaaccgccctgcaaggcggttttttcgttttcagagca | |
| | agagattacgcgcagaccaaaacgatctcaagaagatcatcttattaaggggtctgacgct | |
| | cagtggaacgaaaactcacgttaagggattttggtcatgagattatcaaaaaggatcttca | |
| | cctagatccttttaaattaaaaatgaagttttaaatcaatctaaagtatatatgagtaaac | |
| | ttggtctgacagttaccaatgcttaatcagtgaggcacctatctcagcgatctgtctattt | |
| | cgttcatccatagttgcctgactccccgtcgtgtagataactacgatacgggagggcttac | |
| | catctggccccagtgctgcaatgataccgcgagacccacgctcaccggctccagatttatc | |
| | agcaataaaccagccagccgattcgagctcgcccggggatcgaccagttggtgattttgaa | |
| | cttttgctttgccacggaacggtctgcgttgtcgggaagatgcgtgatctgatccttcaac | |
| | tcagcaaaagttcgatttattcaacaaagccgccgtcccgtcaagtcagcgtaatgctctg | |
| | ccagtgttacaaccaattaaccaattctgattagaaaaactcatcgagcatcaaatgaaac | |
| | tgcaatttattcatatcaggattatcaataccatatttttgaaaaagccgtttctgtaatg | |
| | aaggagaaaactcaccgaggcagttccataggatggcaagatcctggtatcggtctgcgat | |
| | tccgactcgtccaacatcaatacaacctattaatttcccctcgtcaaaaataaggttatca | |
| | agtgagaaatcaccatgagtgacgactgaatccggtgagaatggcaaaagcttatgcattt | |
| | ctttccagacttgttcaacaggccagccattacgctcgtcatcaaaatcactcgcatcaac | |
| | caaaccgttattcattcgtgattgcgcctgagcgagacgaaatacgcgatcgctgttaaaa | |
| | ggacaattacaaacaggaatcgaatgcaaccggcgcaggaacactgccagcgcatcaacaa | |
| | tattttcacctgaatcaggatattcttctaatacctggaatgctgttttcccggggatcgc | |
| | agtggtgagtaaccatgcatcatcaggagtacggataaaatgcttgatggtcggaagaggc | |
| | ataaattccgtcagccagtttagtctgaccatctcatctgtaacatcattggcaacgctac | |
| | ctttgccatgtttcagaaacaactctggcgcatcgggcttcccatacaatcgatagattgt | |
| | cgcacctgattgcccgacattatcgcgagcccatttatacccatataaatcagcatccatg | |
| | ttggaatttaatcgcggcctcgagcaagacgtttcccgttgaatatggctcataacccccc | |
| | ttgtattactgtttatgtaagcagacagttttattgttcatgatgatatatttttatcttg | |
| | tgcaatgtaacatcagagattttgaaacacaacgtggctttcccccccccccccctgcagg | |
| | tctcgggctattcttttgatttataagggattttgccgatttcggcctattggttaaaaaa | |
| | tgagctgatttaacaaaaatttaacgcgaattttaacaaaatattaacgtttacaatttaa | |
| | atatttgcttatacaatcttcctgtttttggggcttttcttattatcaaccggggtacat | |
| PD | tgtcagccgttaagtgttcctgtgtcactcaaaattgctttgagaggctctaagggcttct | 101 |
| | cagtgcgttacatccctggcttgttgtccacaaccgttaaaccttaaaagctttaaaagcc | |
| | ttatatattcttttttttcttataaaacttaaaaccttagaggctatttaagttgctgatt | |
| | tatattaattttattgttcaaacatgagagcttagtacgttagccatgagggtttagttcg | |
| | ttaaacatgagagcttagtacgttaaacatgagagcttagtacgtgaaacatgagagctta | |
| | gtacgttaaacatgagagcttagtacgtgaaacatgagagcttagtacgtactatcaacag | |
| | gttgaactgctgatcttcagatcagagcggatccgtggccggccagccgctccaccgtcaa | |
| | aagaatagcccgagatagggttgagtgttgttccagtttggaacaagagtccactattaaa | |
| | gaacgtggactccaacgtcaaagggcgaaaaaccgtctatcagggcgatggcccactacgt | |
| | gaaccatcaccctaatcaagttttttggggtcgaggtgccgtaaagcactaaatcggaacc | |
| | ctaaagggagcccccgatttagagcttgacggggaaagccggcgaacgtggcgagaaagga | |
| | agggaagaaagcgaaaggagcgggcgctagggcgctggcaagtgtagcggtcacgctgcgc | |
| | gtaaccaccacacccgccgcgcttaatgcgccgctacagggcgcgtgctgaggagacttag | |
| | ggaccctggtttcccgactggaaagcgggcagtgagcgcaacgcaattaatgtgagttagc | |
| | tcactcattaggcaccccaggctttacactttatgcttccggctcgtatgttgtgtggaat | |
| | tgtgagcggataacaatttcacacacctgcaggtgcagtagggagtccacaatggtcagta | |
| | aaggagaagaacttttcactggagttgtcccaattcttgttgaattagatggtgatgttaa | |
| | tgggcacaaattttctgtcagtggagagggtgaaggtgatgcaacatacggaaaacttacc | |
| | cttaaactgatttgcactactggaaaactacctgttccatggccaacacttgtcactactc | |
| | tgggctatggtctgcaatgctttgccagatacccagatcatatgaaacagcatgacttttt | |
| | caagagtgccatgcccgaaggttatgtacaggaaagaactatatttttcaaagatgacggg | |
| | aactacaagacacgtgctgaagtcaagtttgaaggtgatacccttgttaatagaatcgagt | |
| | taaaaggtattgattttaaagaagatggaaacattcttggacacaaattggaatacaacta | |
| | taactcacacaatgtatacatcaccgcagacaaacaaaagaatggaatcaaagccaacttc | |
| | aaaattagacacaacattgaagatggaggcgttcaactagcagaccattatcaacaaaata | |
| | ctccaattggcgatggccctgtccttttaccagacaaccattacctgtcctaccaatctgc | |
| | cctttcgaaagatcccaacgaaaagagagaccacatggtccttcttgagtttgtaacagct | |
| | gctgggattacacatggcatggatgaactatacaaataaacttaattaacggcactcctca | |
| | gccaagtcaaaagcctccgaccggaggcttttgactacatgcccatggcgtttacgccccg | |
| | ccctgccactcatcgcagtactgttgtaattcattaagcattctgccgacatggaagccat | |
| | cacaaacggcatgatgaacctgaatcgccagcggcatcagcaccttgtcgccttgcgtata | |
| | atatttgcccatagtgaaaacgggggcgaagaagttgtccatattggccacgtttaaatca | |
| | aaactggtgaaactcacccagggattggctgagacgaaaaacatattctcaataaaccctt | |
| | tagggaaataggccaggttttcaccgtaacacgccacatcttgcgaatatatgtgtagaaa | |
| | ctgccggaaatcgtcgtggtattcactccagagcgatgaaaacgtttcagtttgctcatgg | |
| | aaaacggtgtaacaagggtgaacactatcccatatcaccagctcaccgtctttcattgcca | |
| | tacggaactccggatgagcattcatcaggcgggcaagaatgtgaataaaggccggataaaa | |
| | cttgtgcttatttttctttacggtctttaaaaaggccgtaatatccagctgaacggtctgg | |
| | ttataggtacattgagcaactgactgaaatgcctcaaaatgttctttacgatgccattggg | |
| | atatatcaacggtggtatatccagtgatttttttctccattttagcttccttagctcctga | |
| | aaatctcgataactcaaaaaatacgcccggtagtgatcttatttcattatggtgaaagttg | |
| | gaacctcttacgtgccaggccaaataggccgt | |
| EC_{H3} | TTTAACAATTTATCAGACAATCTGTGTGGGCACTCGAAGATACGGATTCTTAACGTCGCAA | 102 |
| | GACGAAAAATGAATACCAAGTCTCAAGAGTGAACACGTAATTCATTACGAAGTTTAATTCT | |
| | TTGAGCGTCAAACTTTTAAATTGAAGAGTTTGATCATGGCTCAGATTGAACGCTGGCGGCA | |
| | GGCCTAACACATGCAAGTCGAACGGTAACAGGAAGAAGCTTGCTTCTTTGCTGACGAGTGG | |
| | CGGACGGGTGAGTAATGTCTGGGAAACTGCCTGATGGAGGGGGATAACTACTGGAAACGGT | |
| | AGCTAATACCGCATAACGTCGCAAGACCAAAGAGGGGGACCTTCGGGCCTCTTGCCATCGG | |
| | ATGTGCCCAGATGGGATTAGCTAGTAGGTGGGGTAACGGCTCACCTAGGCGACGATCCCTA | |
| | GCTGGTCTGAGAGGATGACCAGCCACACTGGAACTGAGACACGGTCCAGACTCCTACGGGA | |
| | GGCAGCAGTGGGGAATATTGCACAATGGGCGCAAGCCTGATGCAGCCATGCCGCGTGTATG | |
| | AAGAAGGCCTTCGGGTTGTAAAGTACTTTCAGCGGGGAGGAAGGGAGTAAAGTTAATACCT | |
| | TTGCTCATTGACGTTACCCGCAGAAGAAGCACCGGCTAACTCCGTGCCAGCAGCCGCGGTA | |
| | ATACGGAGGGTGCAAGCGTTAATCGGAATTACTGGGCGTAAAGCGCACGCAGGCGGTTTGT | |
| | TAAGTCAGATGTGAAATCCCCGGGCTCAACCTGGGAACTGCATCTGATACTGGCAAGCTTG | |
| | AGTCTCGTAGAGGGGGGTAGAATTCCAGGTGTAGCGGTGAAATGCGTAGAGATCTGGAGGA | |
| | ATACCGGTGGCGAAGGCGGCCCCCTGGACGAAGACTGACGCTCAGGTGCGAAAGCGTGGGG | |
| | AGCAAACAGGATTAGATACCCTGGTAGTCCACGCCGTAAACGATGTCGACTTGGAGGTTGT | |
| | GCCCTTGAGGCGTGGCTTCCGGAGCTAACGCGTTAAGTCGACCGCCTGGGGAGTACGGCCG | |
| | CAAGGTTAAAACTCAAATGAATTGACGGGGGCCCGCACAAGCGGTGGAGCATGTGGTTTAA | |
| | TTCGATGCAACGCGAAGAACCTTACCTGGTCTTGACATCCACGGAAGTTTTCAGAGATGAG | |
| | AATGTGCCTTCGGGAACCGTGAGACAGGTGCTGCATGGCTGTCGTCAGCTCGTGTTGTGAA | |
| | ATGTTGGGTTAAGTCCCGCAACGAGCGCAACCCTTATCCTTTGTTGCCAGCGGTCCGGCCG | |
| | GGAACTCAAAGGAGACTGCCAGTGATAAACTGGAGGAAGGTGGGGATGACGTCAAGTCATC | |
| | ATGGCCCTTACGACCAGGGCTACACACGTGCTACAATGGCGCATACAAAGAGAAGCGACCT | |
| | CGCGAGAGCAAGCGGACCTCATAAAGTGCGTCGTAGTCCGGATTGGAGTCTGCAACTCGAC | |
| | TCCATGAAGTCGGAATCGCTAGTAATCGTGGATCAGAATGCCACGGTGAATACGTTCCCGG | |
| | GCCTTGTACACACCGCCCGTCACACCATGGGAGTGGGTTGCAAAAGAAGTAGGTAGCTTAA | |
| | CCTTCGGGAGGGCGCTTACCACTTTGTGATTCATGACTGGGGTGAAGTCGTAACAAGGTAA | |
| | CCGTAGGGGAACCTGCGGTTGGATCATGTATATACCTTAAAGAAGCGTACTTTGTAGTGCT | |
| | CACACAGATTGTCTGATAGAAAGTGAAAAGCAAGGCGTTTACGCGTTGGGAGTGAGGC | |
| EC_{H3-1} | TTTAACAATTTATCAGACAATCTGTGTGGGCACTCGAAGATACGGATTCTTAACGTCGCAA | 103 |
| | GACGAAAAATGAATACCAAGTCTCAAGAGTGAACACGTAATTCATTACGAAGTTTAATTCT | |
| | TTGAGCGTCAAACTTTTAAATTGAAGAGTTTGATCATGGCTCAGATTGAACGCTGGCGGCA | |
| | GGCCTAACACATGCAAGTCGAACGGTAACAGGAAGAAGCTTGCTTCTTTGCTGACGAGTGG | |
| | CGGACGGGTGAGTAATGTCTGGGAAACTGCCTGATGGAGGGGGATAACTACTGGAAACGGT | |
| | AGCTAATACCGCATAACGTCGCAAGACCAAAGAGGGGGACCTTCGGGCCTCTTGCCATCGG | |
| | ATGTGCCCAGATGGGATTAGCTAGTAGGTGGGGTAACGGCTCACCTAGGCGACGATCCCTA | |
| | GCTGGTCTGAGAGGATGACCAGCCACACTGGAACTGAGACACGGTCCAGACTCCTACGGGA | |
| | GGCAGCAGTGGGGAATATTGCACAATGGGCGCAAGCCTGATGCAGCCATGCCGCGTGTATG | |
| | AAGAAGGCCTTCGGGTTGTAAAGTACTTTCAGCGGGGAGGAAGGGAGTAAAGTTAATACCT | |
| | TTGCTCATTGACGTTACCCGCAGAAGAAGCACCGGCTAACTCCGTGCCAGCAGCCGCGGTA | |
| | ATACGGAGGGTGCAAGCGTTAATCGGAATTACTGGGCGTAAAGCGCACGCAGGCGGTTTGT | |
| | TAAGTCAGATGTGAAATCCCCGGGCTCAACCTGGGAACTGCATCTGATACTGGCAAGCTTG | |
| | AGTCTCGTAGAGGGGGGTAGAATTCCAGGTGTAGCGGTGAAATGCGTAGAGATCTGGAGGA | |
| | ATACCGGTGGCGAAGGCGGCCCCCTGGACGAAGACTGACGCTCAGGTGCGAAAGCGTGGGG | |
| | AGCAAACAGGATTAGATACCCTGGTAGTCCACGCCGTAAACGATGTCGACTTGGAGGTTGT | |
| | GCCCTTGAGGCGTGGCTTCCGGAGCTAACGCGTTAAGTCGACCGCCTGGGGAGTACGGCCG | |
| | CAAGGTTAAAACTCAAATGAATTGACGGGGGCCCGCACAAGCGGTGGAGCATGTGGTTTAA | |
| | TTCGATGCAACGCGAAGAACCTTACCTGGTCTTGACATCCACGGAAGTTTTCAGAGATGAG | |
| | AATGTGCCTTCGGGAACCGTGAGACAGGTGCTGCATGGCTGTCGTCAGCTCGTGTTGTGAA | |
| | ATGTTGGGTTAAGTCCCGCAACGAGCGCAACCCTTATCCTTTGTTGCCAGCGGTCCGGCCG | |
| | GGAACTCAAAGGAGACTGCCAGTGATAAACTGGAGGAAGGTGGGGATGACGTCAAGTCATC | |
| | ATGGCCCTTACGACCAGGGCTACACACGTGCTACAATGGCGCATACAAAGAGAAGCGACCT | |
| | CGCGAGAGCAAGCGGACCTCATAAAGTGCGTCGTAGTCCGGATTGGAGTCTGCAACTCGAC | |
| | TCCATGAAGTCGGAATCGCTAGTAATCGTGGATCAGAATGCCACGGTGAATACGTTCCCGG | |
| | GCCTTGTACACACCGCCCGTCACACCATGGGAGTGGGTTGCAAAAGAAGTAGGTAGCTTAA | |
| | CCTTCGGGAGGGCGCTTACCACTTTGTGATTCATGACTGGGGTGAAGTCGTAACAAGGTAA | |
| | CCGTAGGGGAACCTGCGGTTGGATCACTTGTATACCTTAAAGAAGCGTACTTTGTAGTGCT | |
| | CACACAGATTGTCTGATAGAAAGTGAAAAGCAAGGCGTTTACGCGTTGGGAGTGAGGCTGA | |
| | AGAGAATAAGGCCGTTCGCTTTCTATTAATGAAAGCTCACCCTACACGAAAATATCACGCA | |
| | ACGCGTGATAAGCAATTTTCGTGTCCCCTTCGTCTAGAGGCCCAGGACACCGCCCTTTCAC | |
| | GGCGGTAACAGGGGTTCGAATCCCCTAGGGGACGCCACTTGCTGGTTTGTGAGTGAAAGTC | |
| | GCCGACCTTAATATCTCAAAACTCATCTTCGGGTGATGTTTGAGATATTTGCTCTTTAAAA | |
| | ATCTGGATCAAGCTGAAAATTGAAACACTGAACAACGAGAGTTGTTCGTGAGTCTCTCAAA | |
| | TTTTCGCAACACGATGATGAATCGAAAGAAACATCTTCGGGTTGTGAGGTTAAGCGACTAA | |
| | GCGTACACGGTGGATGCCCTGGCAGTCAGAGGCGATGAAGGACGTGCTAATCTGCGATAAG | |
| | CGTCGGTAAGGTGATATGAACCGTTATAACCGGCGATTTCCGAATGGGGAAACCCAGTGTG | |
| | TTTCGACACACTATCATTAACTGAATCCATAGGTTAATGAGGCGAACCGGGGGAACTGAAA | |
| | CATCTAAGTACCCCGAGGAAAAGAAATCAACCGAGATTCCCCCAGTAGCGGCGAGCGAACG | |
| | GGGAGCAGCCCAGAGCCTGAATCAGTGTGTGTGTTAGTGGAAGCGTCTGGAAAGGCGCGCG | |
| | ATACAGGGTGACAGCCCCGTACACAAAAATGCACATGCTGTGAGCTCGATGAGTAGGGCGG | |
| | GACACGTGGTATCCTGTCTGAATATGGGGGGACCATCCTCCAAGGCTAAATACTCCTGACT | |
| | GACCGATAGTGAACCAGTACCGTGAGGGAAAGGCGAAAAGAACCCCGGCGAGGGGAGTGAA | |
| | AAAGAACCTGAAACCGTGTACGTACAAGCAGTGGGAGCACGCTTAGGCGTGTGACTGCGTA | |
| | CCTTTTGTATAATGGGTCAGCGACTTATATTCTGTAGCAAGGTTAACCGAATAGGGGAGCC | |
| | GAAGGGAAACCGAGTCTTAACTGGGCGTTAAGTTGCAGGGTATAGACCCGAAACCCGGTGA | |
| | TCTAGCCATGGGCAGGTTGAAGGTTGGGTAACACTAACTGGAGGACCGAACCGACTAATGT | |
| | TGAAAAATTAGCGGATGACTTGTGGCTGGGGGTGAAAGGCCAATCAAACCGGGAGATAGCT | |
| | GGTTCTCCCCGAAAGCTATTTAGGTAGCGCCTCGTGAATTCATCTCCGGGGGTAGAGCACT | |
| | GTTTCGGCAAGGGGGTCATCCCGACTTACCAACCCGATGCAAACTGCGAATACCGGAGAAT | |
| | GTTATCACGGGAGACACACGGCGGGTGCTAACGTCCGTCGTGAAGAGGGAAACAACCCAGA | |
| | CCGCCAGCTAAGGTCCCAAAGTCATGGTTAAGTGGGAAACGATGTGGGAAGGCCCAGACAG | |
| | CCAGGATGTTGGCTTAGAAGCAGCCATCATTTAAAGAAAGCGTAATAGCTCACTGGTCGAG | |
| | TCGGCCTGCGCGGAAGATGTAACGGGGCTAAACCATGCACCGAAGCTGCGGCAGCGACGCT | |
| | TATGCGTTGTTGGGTAGGGGAGCGTTCTGTAAGCCTGCGAAGGTGTGCTGTGAGGCATGCT | |
| | GGAGGTATCAGAAGTGCGAATGCTGACATAAGTAACGATAAAGCGGGTGAAAAGCCCGCTC | |
| | GCCGGAAGACCAAGGGTTCCTGTCCAACGTTAATCGGGGCAGGGTGAGTCGACCCCTAAGG | |
| | CGAGGCCGAAAGGCGTAGTCGATGGGAAACAGGTTAATATTCCTGTACTTGGTGTTACTGC | |
| | GAAGGGGGGACGGAGAAGGCTATGTTGGCCGGGCGACGGTTGTCCCGGTTTAAGCGTGTAG | |
| | GCTGGTTTTCCAGGCAAATCCGGAAAATCAAGGCTGAGGCGTGATGACGAGGCACTACGGT | |
| | GCTGAAGCAACAAATGCCCTGCTTCCAGGAAAAGCCTCTAAGCATCAGGTAACATCAAATC | |
| | GTACCCCAAACCGACACAGGTGGTCAGGTAGAGAATACCAAGGCGCTTGAGAGAACTCGGG | |
| | TGAAGGAACTAGGCAAAATGGTGCCGTAACTTCGGGAGAAGGCACGCTGATATGTAGGTGA | |
| | GGTCCCTCGCGGATGGAGCTGAAATCAGTCGAAGATACCAGCTGGCTGCAACTGTTTATTA | |
| | AAAACACAGCACTGTGCAAACACGAAAGTGGACGTATACGGTGTGACGCCTGCCCGGTGCC | |
| | GGAAGGTTAATTGATGGGGTTAGCGCAAGCGAAGCTCTTGATCGAAGCCCCGGTAAACGGC | |
| | GGCCGTAACTATAACGGTCCTAAGGTAGCGAAATTCCTTGTCGGGTAAGTTCCGACCTGCA | |
| | CGAATGGCGTAATGATGGCCAGGCTGTCTCCACCCGAGACTCAGTGAAATTGAACTCGCTG | |
| | TGAAGATGCAGTGTACCCGCGGCAAGACGGAAAGACCCCGTGAACCTTTACTATAGCTTGA | |
| | CACTGAACATTGAGCCTTGATGTGTAGGATAGGTGGGAGGCTTTGAAGTGTGGACGCCAGT | |
| | CTGCATGGAGCCGACCTTGAAATACCACCCTTTAATGTTTGATGTTCTAACGTTGACCCGT | |
| | AATCCGGGTTGCGGACAGTGTCTGGTGGGTAGTTTGACTGGGGCGGTCTCCTCCTAAAGAG | |
| | TAACGGAGGAGCACGAAGGTTGGCTAATCCTGGTCGGACATCAGGAGGTTAGTGCAATGGC | |
| | ATAAGCCAGCTTGACTGCGAGCGTGACGGCGCGAGCAGGTGCGAAAGCAGGTCATAGTGAT | |
| | CCGGTGGTTCTGAATGGAAGGGCCATCGCTCAACGGATAAAAGGTACTCCGGGGATAACAG | |
| | GCTGATACCGCCCAAGAGTTCATATCGACGGCGGTGTTTGGCACCTCGATGTCGGCTCATC | |
| | ACATCCTGGGGCTGAAGTAGGTCCCAAGGGTATGGCTGTTCGCCATTTAAAGTGGTACGCG | |
| | AGCTGGGTTTAGAACGTCGTGAGACAGTTCGGTCCCTATCTGCCGTGGGCGCTGGAGAACT | |
| | GAGGGGGGCTGCTCCTAGTACGAGAGGACCGGAGTGGACGCATCACTGGTGTTCGGGTTGT | |
| | CATGCCAATGGCACTGCCCGGTAGCTAAATGCGGAAGAGATAAGTGCTGAAAGCATCTAAG | |
| | CACGAAACTTGCCCCGAGATGAGTTCTCCCTGACCCTTTAAGGGTCCTGAAGGAACGTTGA | |
| | AGACGACGACGTTGATAGGCCGGGTGTGTAAGCGCAGCGATGCGTTGAGCTAACCGGTACT | |
| | AATGAACCGTGAGGCTTAACCTTACAACGCCGAAGCTGTTTTGGCGGATGAGAGAAGATTT | |
| | TCAGCCTGATACAGATTAAATCAGAACGCAGAAGCGGTCTGATAAAACAGAATTTGCCTGG | |
| | CGGCAGTAGCGCGGTGGTCCCACCTGACCCCATGCCGAACTCAGAAGTGAAACGCCGTAGC | |
| | GCCGATGGTAGTGTGGGGTCTCCCCATGCGAGAGTAGGGAACTGCCAGGCATCAAATAAAA | |
| | CGAAAGGCTCAGTCGAAAGACTGGGCCTTTCGTTTTATCTGTTGTTTGTCGGTGAACGCTC | |
| | TCC | |
| EC_{H3-2} | TTTAACAATTTATCAGACAATCTGTGTGGGCACTCGAAGATACGGATTCTTAACGTCGCAA | 104 |
| | GACGAAAAATGAATACCAAGTCTCAAGAGTGAACACGTAATTCATTACGAAGTTTAATTCT | |
| | TTGAGCGTCAAACTTTTAAATTGAAGAGTTTGATCATGGCTCAGATTGAACGCTGGCGGCA | |
| | GGCCTAACACATGCAAGTCGAACGGTAACAGGAAGAAGCTTGCTTCTTTGCTGACGAGTGG | |
| | CGGACGGGTGAGTAATGTCTGGGAAACTGCCTGATGGAGGGGGATAACTACTGGAAACGGT | |
| | AGCTAATACCGCATAACGTCGCAAGACCAAAGAGGGGGACCTTCGGGCCTCTTGCCATCGG | |
| | ATGTGCCCAGATGGGATTAGCTAGTAGGTGGGGTAACGGCTCACCTAGGCGACGATCCCTA | |
| | GCTGGTCTGAGAGGATGACCAGCCACACTGGAACTGAGACACGGTCCAGACTCCTACGGGA | |
| | GGCAGCAGTGGGGAATATTGCACAATGGGCGCAAGCCTGATGCAGCCATGCCGCGTGTATG | |
| | AAGAAGGCCTTCGGGTTGTAAAGTACTTTCAGCGGGGAGGAAGGGAGTAAAGTTAATACCT | |
| | TTGCTCATTGACGTTACCCGCAGAAGAAGCACCGGCTAACTCCGTGCCAGCAGCCGCGGTA | |
| | ATACGGAGGGTGCAAGCGTTAATCGGAATTACTGGGCGTAAAGCGCACGCAGGCGGTTTGT | |
| | TAAGTCAGATGTGAAATCCCCGGGCTCAACCTGGGAACTGCATCTGATACTGGCAAGCTTG | |
| | AGTCTCGTAGAGGGGGGTAGAATTCCAGGTGTAGCGGTGAAATGCGTAGAGATCTGGAGGA | |
| | ATACCGGTGGCGAAGGCGGCCCCCTGGACGAAGACTGACGCTCAGGTGCGAAAGCGTGGGG | |
| | AGCAAACAGGATTAGATACCCTGGTAGTCCACGCCGTAAACGATGTCGACTTGGAGGTTGT | |
| | GCCCTTGAGGCGTGGCTTCCGGAGCTAACGCGTTAAGTCGACCGCCTGGGGAGTACGGCCG | |
| | CAAGGTTAAAACTCAAATGAATTGACGGGGGCCCGCACAAGCGGTGGAGCATGTGGTTTAA | |
| | TTCGATGCAACGCGAAGAACCTTACCTGGTCTTGACATCCACGGAAGTTTTCAGAGATGAG | |
| | AATGTGCCTTCGGGAACCGTGAGACAGGTGCTGCATGGCTGTCGTCAGCTCGTGTTGTGAA | |
| | ATGTTGGGTTAAGTCCCGCAACGAGCGCAACCCTTATCCTTTGTTGCCAGCGGTCCGGCCG | |
| | GGAACTCAAAGGAGACTGCCAGTGATAAACTGGAGGAAGGTGGGGATGACGTCAAGTCATC | |
| | ATGGCCCTTACGACCAGGGCTACACACGTGCTACAATGGCGCATACAAAGAGAAGCGACCT | |
| | CGCGAGAGCAAGCGGACCTCATAAAGTGCGTCGTAGTCCGGATTGGAGTCTGCAACTCGAC | |
| | TCCATGAAGTCGGAATCGCTAGTAATCGTGGATCAGAATGCCACGGTGAATACGTTCCCGG | |
| | GCCTTGTACACACCGCCCGTCACACCATGGGAGTGGGTTGCAAAAGAAGTAGGTAGCTTAA | |
| | CCTTCGGGAGGGCGCTTACCACTTTGTGATTCATGACTGGGGTGAAGTCGTAACAAGGTAA | |
| | CCGTAGGGGAACCTGCGGTTGGATCAATGTATTACCTTAAAGAAGCGTACTTTGTAGTGCT | |
| | CACACAGATTGTCTGATAGAAAGTGAAAAGCAAGGCGTTTACGCGTTGGGAGTGAGGCTGA | |
| | AGAGAATAAGGCCGTTCGCTTTCTATTAATGAAAGCTCACCCTACACGAAAATATCACGCA | |
| | ACGCGTGATAAGCAATTTTCGTGTCCCCTTCGTCTAGAGGCCCAGGACACCGCCCTTTCAC | |
| | GGCGGTAACAGGGGTTCGAATCCCCTAGGGGACGCCACTTGCTGGTTTGTGAGTGAAAGTC | |
| | GCCGACCTTAATATCTCAAAACTCATCTTCGGGTGATGTTTGAGATATTTGCTCTTTAAAA | |
| | ATCTGGATCAAGCTGAAAATTGAAACACTGAACAACGAGAGTTGTTCGTGAGTCTCTCAAA | |
| | TTTTCGCAACACGATGATGAATCGAAAGAAACATCTTCGGGTTGTGAGGTTAAGCGACTAA | |
| | GCGTACACGGTGGATGCCCTGGCAGTCAGAGGCGATGAAGGACGTGCTAATCTGCGATAAG | |
| | CGTCGGTAAGGTGATATGAACCGTTATAACCGGCGATTTCCGAATGGGGAAACCCAGTGTG | |
| | TTTCGACACACTATCATTAACTGAATCCATAGGTTAATGAGGCGAACCGGGGGAACTGAAA | |
| | CATCTAAGTACCCCGAGGAAAAGAAATCAACCGAGATTCCCCCAGTAGCGGCGAGCGAACG | |
| | GGGAGCAGCCCAGAGCCTGAATCAGTGTGTGTGTTAGTGGAAGCGTCTGGAAAGGCGCGCG | |
| | ATACAGGGTGACAGCCCCGTACACAAAAATGCACATGCTGTGAGCTCGATGAGTAGGGCGG | |
| | GACACGTGGTATCCTGTCTGAATATGGGGGGACCATCCTCCAAGGCTAAATACTCCTGACT | |
| | GACCGATAGTGAACCAGTACCGTGAGGGAAAGGCGAAAAGAACCCCGGCGAGGGGAGTGAA | |
| | AAAGAACCTGAAACCGTGTACGTACAAGCAGTGGGAGCACGCTTAGGCGTGTGACTGCGTA | |
| | CCTTTTGTATAATGGGTCAGCGACTTATATTCTGTAGCAAGGTTAACCGAATAGGGGAGCC | |
| | GAAGGGAAACCGAGTCTTAACTGGGCGTTAAGTTGCAGGGTATAGACCCGAAACCCGGTGA | |
| | TCTAGCCATGGGCAGGTTGAAGGTTGGGTAACACTAACTGGAGGACCGAACCGACTAATGT | |
| | TGAAAAATTAGCGGATGACTTGTGGCTGGGGGTGAAAGGCCAATCAAACCGGGAGATAGCT | |
| | GGTTCTCCCCGAAAGCTATTTAGGTAGCGCCTCGTGAATTCATCTCCGGGGGTAGAGCACT | |
| | GTTTCGGCAAGGGGGTCATCCCGACTTACCAACCCGATGCAAACTGCGAATACCGGAGAAT | |
| | GTTATCACGGGAGACACACGGCGGGTGCTAACGTCCGTCGTGAAGAGGGAAACAACCCAGA | |
| | CCGCCAGCTAAGGTCCCAAAGTCATGGTTAAGTGGGAAACGATGTGGGAAGGCCCAGACAG | |
| | CCAGGATGTTGGCTTAGAAGCAGCCATCATTTAAAGAAAGCGTAATAGCTCACTGGTCGAG | |
| | TCGGCCTGCGCGGAAGATGTAACGGGGCTAAACCATGCACCGAAGCTGCGGCAGCGACGCT | |
| | TATGCGTTGTTGGGTAGGGGAGCGTTCTGTAAGCCTGCGAAGGTGTGCTGTGAGGCATGCT | |
| | GGAGGTATCAGAAGTGCGAATGCTGACATAAGTAACGATAAAGCGGGTGAAAAGCCCGCTC | |
| | GCCGGAAGACCAAGGGTTCCTGTCCAACGTTAATCGGGGCAGGGTGAGTCGACCCCTAAGG | |
| | CGAGGCCGAAAGGCGTAGTCGATGGGAAACAGGTTAATATTCCTGTACTTGGTGTTACTGC | |
| | GAAGGGGGGACGGAGAAGGCTATGTTGGCCGGGCGACGGTTGTCCCGGTTTAAGCGTGTAG | |
| | GCTGGTTTTCCAGGCAAATCCGGAAAATCAAGGCTGAGGCGTGATGACGAGGCACTACGGT | |
| | GCTGAAGCAACAAATGCCCTGCTTCCAGGAAAAGCCTCTAAGCATCAGGTAACATCAAATC | |
| | GTACCCCAAACCGACACAGGTGGTCAGGTAGAGAATACCAAGGCGCTTGAGAGAACTCGGG | |
| | TGAAGGAACTAGGCAAAATGGTGCCGTAACTTCGGGAGAAGGCACGCTGATATGTAGGTGA | |
| | GGTCCCTCGCGGATGGAGCTGAAATCAGTCGAAGATACCAGCTGGCTGCAACTGTTTATTA | |
| | AAAACACAGCACTGTGCAAACACGAAAGTGGACGTATACGGTGTGACGCCTGCCCGGTGCC | |
| | GGAAGGTTAATTGATGGGGTTAGCGCAAGCGAAGCTCTTGATCGAAGCCCCGGTAAACGGC | |
| | GGCCGTAACTATAACGGTCCTAAGGTAGCGAAATTCCTTGTCGGGTAAGTTCCGACCTGCA | |
| | CGAATGGCGTAATGATGGCCAGGCTGTCTCCACCCGAGACTCAGTGAAATTGAACTCGCTG | |
| | TGAAGATGCAGTGTACCCGCGGCAAGACGGAAAGACCCCGTGAACCTTTACTATAGCTTGA | |
| | CACTGAACATTGAGCCTTGATGTGTAGGATAGGTGGGAGGCTTTGAAGTGTGGACGCCAGT | |
| | CTGCATGGAGCCGACCTTGAAATACCACCCTTTAATGTTTGATGTTCTAACGTTGACCCGT | |
| | AATCCGGGTTGCGGACAGTGTCTGGTGGGTAGTTTGACTGGGGCGGTCTCCTCCTAAAGAG | |
| | TAACGGAGGAGCACGAAGGTTGGCTAATCCTGGTCGGACATCAGGAGGTTAGTGCAATGGC | |
| | ATAAGCCAGCTTGACTGCGAGCGTGACGGCGCGAGCAGGTGCGAAAGCAGGTCATAGTGAT | |
| | CCGGTGGTTCTGAATGGAAGGGCCATCGCTCAACGGATAAAAGGTACTCCGGGGATAACAG | |
| | GCTGATACCGCCCAAGAGTTCATATCGACGGCGGTGTTTGGCACCTCGATGTCGGCTCATC | |
| | ACATCCTGGGGCTGAAGTAGGTCCCAAGGGTATGGCTGTTCGCCATTTAAAGTGGTACGCG | |
| | AGCTGGGTTTAGAACGTCGTGAGACAGTTCGGTCCCTATCTGCCGTGGGCGCTGGAGAACT | |
| | GAGGGGGGCTGCTCCTAGTACGAGAGGACCGGAGTGGACGCATCACTGGTGTTCGGGTTGT | |
| | CATGCCAATGGCACTGCCCGGTAGCTAAATGCGGAAGAGATAAGTGCTGAAAGCATCTAAG | |
| | CACGAAACTTGCCCCGAGATGAGTTCTCCCTGACCCTTTAAGGGTCCTGAAGGAACGTTGA | |
| | AGACGACGACGTTGATAGGCCGGGTGTGTAAGCGCAGCGATGCGTTGAGCTAACCGGTACT | |
| | AATGAACCGTGAGGCTTAACCTTACAACGCCGAAGCTGTTTTGGCGGATGAGAGAAGATTT | |
| | TCAGCCTGATACAGATTAAATCAGAACGCAGAAGCGGTCTGATAAAACAGAATTTGCCTGG | |
| | CGGCAGTAGCGCGGTGGTCCCACCTGACCCCATGCCGAACTCAGAAGTGAAACGCCGTAGC | |
| | GCCGATGGTAGTGTGGGGTCTCCCCATGCGAGAGTAGGGAACTGCCAGGCATCAAATAAAA | |
| | CGAAAGGCTCAGTCGAAAGACTGGGCCTTTCGTTTTATCTGTTGTTTGTCGGTGAACGCTC | |
| | TCC | |
| Δh6 | GCGGCCGCGATCTCTCACCTACCAAACAATGCCCCCCTGCAAAAAATAAATTCATATAAAA | 105 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACttGACGAGTGGCGGACGGGTGAGTAATGTCTGGGA | |
| | AACTGCCTGATGGAGGGGGATAACTACTGGAAACGGTAGCTAATACCGCATAACGTCGCAA | |
| | GACCAAAGAGGGGGACCTTCGGGCCTCTTGCCATCGGATGTGCCCAGATGGGATTAGCTAG | |
| | TAGGTGGGGTAACGGCTCACCTAGGCGACGATCCCTAGCTGGTCTGAGAGGATGACCAGCC | |
| | ACACTGGAACTGAGACACGGTCCAGACTCCTACGGGAGGCAGCAGTGGGGAATATTGCACA | |
| | ATGGGCGCAAGCCTGATGCAGCCATGCCGCGTGTATGAAGAAGGCCTTCGGGTTGTAAAGT | |
| | ACTTTCAGCGGGGAGGAAGGGAGTAAAGTTAATACCTTTGCTCATTGACGTTACCCGCAGA | |
| | AGAAGCACCGGCTAACTCCGTGCCAGCAGCCGCGGTAATACGGAGGGTGCAAGCGTTAATC | |
| | GGAATTACTGGGCGTAAAGCGCACGCAGGCGGTTTGTTAAGTCAGATGTGAAATCCCCGGG | |
| | CTCAACCTGGGAACTGCATCTGATACTGGCAAGCTTGAGTCTCGTAGAGGGGGGTAGAATT | |
| | CCAGGTGTAGCGGTGAAATGCGTAGAGATCTGGAGGAATACCGGTGGCGAAGGCGGCCCCC | |
| | TGGACGAAGACTGACGCTCAGGTGCGAAAGCGTGGGGAGCAAACAGGATTAGATACCCTGG | |
| | TAGTCCACGCCGTAAACGATGTCGACTTGGAGGTTGTGCCCTTGAGGCGTGGCTTCCGGAG | |
| | CTAACGCGTTAAGTCGACCGCCTGGGGAGTACGGCCGCAAGGTTAAAACTCAAATGAATTG | |
| | ACGGGGGCCCGCACAAGCGGTGGAGCATGTGGTTTAATTCGATGCAACGCGAAGAACCTTA | |
| | CCTGGTCTTGACATCCACGGAAGTTTTCAGAGATGAGAATGTGCCTTCGGGAACCGTGAGA | |
| | CAGGTGCTGCATGGCTGTCGTCAGCTCGTGTTGTGAAATGTTGGGTTAAGTCCCGCAACGA | |
| | GCGCAACCCTTATCCTTTGTTGCCAGCGGTCCGGCCGGGAACTCAAAGGAGACTGCCAGTG | |
| | ATAAACTGGAGGAAGGTGGGGATGACGTCAAGTCATCATGGCCCTTACGACCAGGGCTACA | |
| | CACGTGCTACAATGGCGCATACAAAGAGAAGCGACCTCGCGAGAGCAAGCGGACCTCATAA | |
| | AGTGCGTCGTAGTCCGGATTGGAGTCTGCAACTCGACTCCATGAAGTCGGAATCGCTAGTA | |
| | ATCGTGGATCAGAATGCCACGGTGAATACGTTCCCGGGCCTTGTACACACCGCCCGTCACA | |
| | CCATGGGAGTGGGTTGCAAAAGAAGTAGGTAGCTTAACCTTCGGGAGGGCGCTTACCACTT | |
| | TGTGATTCATGACTGGGGTGAAGTCGTAACAAGGTAACCGTAGGGGAACCTGCGGTTGGAT | |
| | CAtgtggtTACCTTAAAGAAGCGTACTTTGTAGTGCTCACACAGATTGTCTGATAGAAAGT | |
| | GAAAAGCAAGGCGTTTACGCGTTGGGAGTGAGGCTGAAGAGAATAAGGCCGTTCGCTTTCT | |
| | ATTAATGAAAGCTCACCCTACACGAAAATATCACGCAACGCGTGATAAGCAATTTTCGTGT | |
| | CCCCTTCGTCTAGAGGCCCAGGACACCGCCCTTTCACGGCGGTAACAGGGGTTCGAATCCC | |
| | CTAGGGGACGCCACTTGCTGGTTTGTGAGTGAAAGTCGCCGACCTTAATATCTCAAAACTC | |
| | ATCTTCGGGTGATGTTTGAGATATTTGCTCTTTAAAAATCTGGATCAAGCTGAAAATTGAA | |
| | ACACTGAACAACGAGAGTTGTTCGTGAGTCTCTCAAATTTTCGCAACACGATGATGAATCG | |
| | AAAGAAACATCTTCGGGTTGTGAGGTTAAGCGACTAAGCGTACACGGTGGATGCCCTGGCA | |
| | GTCAGAGGCGATGAAGGACGTGCTAATCTGCGATAAGCGTCGGTAAGGTGATATGAACCGT | |
| | TATAACCGGCGATTTCCGAATGGGGAAACCCAGTGTGTTTCGACACACTATCATTAACTGA | |
| | ATCCATAGGTTAATGAGGCGAACCGGGGGAACTGAAACATCTAAGTACCCCGAGGAAAAGA | |
| | AATCAACCGAGATTCCCCCAGTAGCGGCGAGCGAACGGGGAGCAGCCCAGAGCCTGAATCA | |
| | GTGTGTGTGTTAGTGGAAGCGTCTGGAAAGGCGCGCGATACAGGGTGACAGCCCCGTACAC | |
| | AAAAATGCACATGCTGTGAGCTCGATGAGTAGGGCGGGACACGTGGTATCCTGTCTGAATA | |
| | TGGGGGGACCATCCTCCAAGGCTAAATACTCCTGACTGACCGATAGTGAACCAGTACCGTG | |
| | AGGGAAAGGCGAAAAGAACCCCGGCGAGGGGAGTGAAAAAGAACCTGAAACCGTGTACGTA | |
| | CAAGCAGTGGGAGCACGCTTAGGCGTGTGACTGCGTACCTTTTGTATAATGGGTCAGCGAC | |
| | TTATATTCTGTAGCAAGGTTAACCGAATAGGGGAGCCGAAGGGAAACCGAGTCTTAACTGG | |
| | GCGTTAAGTTGCAGGGTATAGACCCGAAACCCGGTGATCTAGCCATGGGCAGGTTGAAGGT | |
| | TGGGTAACACTAACTGGAGGACCGAACCGACTAATGTTGAAAAATTAGCGGATGACTTGTG | |
| | GCTGGGGGTGAAAGGCCAATCAAACCGGGAGATAGCTGGTTCTCCCCGAAAGCTATTTAGG | |
| | TAGCGCCTCGTGAATTCATCTCCGGGGGTAGAGCACTGTTTCGGCAAGGGGGTCATCCCGA | |
| | CTTACCAACCCGATGCAAACTGCGAATACCGGAGAATGTTATCACGGGAGACACACGGCGG | |
| | GTGCTAACGTCCGTCGTGAAGAGGGAAACAACCCAGACCGCCAGCTAAGGTCCCAAAGTCA | |
| | TGGTTAAGTGGGAAACGATGTGGGAAGGCCCAGACAGCCAGGATGTTGGCTTAGAAGCAGC | |
| | CATCATTTAAAGAAAGCGTAATAGCTCACTGGTCGAGTCGGCCTGCGCGGAAGATGTAACG | |
| | GGGCTAAACCATGCACCGAAGCTGCGGCAGCGACGCTTATGCGTTGTTGGGTAGGGGAGCG | |
| | TTCTGTAAGCCTGCGAAGGTGTGCTGTGAGGCATGCTGGAGGTATCAGAAGTGCGAATGCT | |
| | GACATAAGTAACGATAAAGCGGGTGAAAAGCCCGCTCGCCGGAAGACCAAGGGTTCCTGTC | |
| | CAACGTTAATCGGGGCAGGGTGAGTCGACCCCTAAGGCGAGGCCGAAAGGCGTAGTCGATG | |
| | GGAAACAGGTTAATATTCCTGTACTTGGTGTTACTGCGAAGGGGGGACGGAGAAGGCTATG | |
| | TTGGCCGGGCGACGGTTGTCCCGGTTTAAGCGTGTAGGCTGGTTTTCCAGGCAAATCCGGA | |
| | AAATCAAGGCTGAGGCGTGATGACGAGGCACTACGGTGCTGAAGCAACAAATGCCCTGCTT | |
| | CCAGGAAAAGCCTCTAAGCATCAGGTAACATCAAATCGTACCCCAAACCGACACAGGTGGT | |
| | CAGGTAGAGAATACCAAGGCGCTTGAGAGAACTCGGGTGAAGGAACTAGGCAAAATGGTGC | |
| | CGTAACTTCGGGAGAAGGCACGCTGATATGTAGGTGAGGTCCCTCGCGGATGGAGCTGAAA | |
| | TCAGTCGAAGATACCAGCTGGCTGCAACTGTTTATTAAAAACACAGCACTGTGCAAACACG | |
| | AAAGTGGACGTATACGGTGTGACGCCTGCCCGGTGCCGGAAGGTTAATTGATGGGGTTAGC | |
| | GCAAGCGAAGCTCTTGATCGAAGCCCCGGTAAACGGCGGCCGTAACTATAACGGTCCTAAG | |
| | GTAGCGAAATTCCTTGTCGGGTAAGTTCCGACCTGCACGAATGGCGTAATGATGGCCAGGC | |
| | TGTCTCCACCCGAGACTCAGTGAAATTGAACTCGCTGTGAAGATGCAGTGTACCCGCGGCA | |
| | AGACGGAAAGACCCCGTGAACCTTTACTATAGCTTGACACTGAACATTGAGCCTTGATGTG | |
| | TAGGATAGGTGGGAGGCTTTGAAGTGTGGACGCCAGTCTGCATGGAGCCGACCTTGAAATA | |
| | CCACCCTTTAATGTTTGATGTTCTAACGTTGACCCGTAATCCGGGTTGCGGACAGTGTCTG | |
| | GTGGGTAGTTTGACTGGGGCGGTCTCCTCCTAAAGAGTAACGGAGGAGCACGAAGGTTGGC | |
| | TAATCCTGGTCGGACATCAGGAGGTTAGTGCAATGGCATAAGCCAGCTTGACTGCGAGCGT | |
| | GACGGCGCGAGCAGGTGCGAAAGCAGGTCATAGTGATCCGGTGGTTCTGAATGGAAGGGCC | |
| | ATCGCTCAACGGATAAAAGGTACTCCGGGGATAACAGGCTGATACCGCCCAAGAGTTCATA | |
| | TCGACGGCGGTGTTTGGCACCTCGATGTCGGCTCATCACATCCTGGGGCTGAAGTAGGTCC | |
| | CAAGGGTATGGCTGTTCGCCATTTAAAGTGGTACGCGAGCTGGGTTTAGAACGTCGTGAGA | |
| | CAGTTCGGTCCCTATCTGCCGTGGGCGCTGGAGAACTGAGGGGGGCTGCTCCTAGTACGAG | |
| | AGGACCGGAGTGGACGCATCACTGGTGTTCGGGTTGTCATGCCAATGGCACTGCCCGGTAG | |
| | CTAAATGCGGAAGAGATAAGTGCTGAAAGCATCTAAGCACGAAACTTGCCCCGAGATGAGT | |
| | TCTCCCTGACCCTTTAAGGGTCCTGAAGGAACGTTGAAGACGACGACGTTGATAGGCCGGG | |
| | TGTGTAAGCGCAGCGATGCGTTGAGCTAACCGGTACTAATGAACCGTGAGGCTTAACCTTA | |
| | CAACGCCGAAGCTGTTTTGGCGGATGAGAGAAGATTTTCAGCCTGATACAGATTAAATCAG | |
| | AACGCAGAAGCGGTCTGATAAAACAGAATTTGCCTGGCGGCAGTAGCGCGGTGGTCCCACC | |
| | TGACCCCATGCCGAACTCAGAAGTGAAACGCCGTAGCGCCGATGGTAGTGTGGGGTCTCCC | |
| | CATGCGAGAGTAGGGAACTGCCAGGCATCAAATAAAACGAAAGGCTCAGTCGAAAGACTGG | |
| | GCCTTTCGTTTTATCTGTTGTTTGTCGGTGAACGCTCTCCTGAGTAGGACAAATCCGCCGG | |
| | GAGCGGATTTGAACGTTGCGAAGCAACGGCCCGGAGGGTGGCGGGCAGGACGCCCGCCATA | |
| | AACTGCCAGGCATCAAATTAAGCAGAAGGCCATCCTGACGGATGGCCTTTTTGCGTTTCTA | |
| | CAAACTCTTCCTGTCGTCATATCTACAAGCCGGCGCGCCGGGAAATGTGCGCGGAACCCCT | |
| | ATTTGTTTATTTTTCTAAATACATTCAAATATGTATCCGCTCATGAGACAATAACCCTGAT | |
| | AAATGCTTCAATAATATTGAAAAAGGAAGAGTATGAGTATTCAACATTTCCGTGTCGCCCT | |
| | TATTCCCTTTTTTGCGGCATTTTGCCTTCCTGTTTTTGCTCACCCAGAAACGCTGGTGAAA | |
| | GTAAAAGATGCTGAAGATCAGTTGGGTGCACGAGTGGGTTACATCGAACTGGATCTCAACA | |
| | GCGGTAAGATCCTTGAGAGTTTTCGCCCCGAAGAACGTTTTCCAATGATGAGCACTTTTAA | |
| | AGTTCTGCTATGTGGCGCGGTATTATCCCGTGTTGACGCCGGGCAAGAGCAACTCGGTCGC | |
| | CGCATACACTATTCTCAGAATGACTTGGTTGAGTACTCACCAGTCACAGAAAAGCATCTTA | |
| | CGGATGGCATGACAGTAAGAGAATTATGCAGTGCTGCAATAACCATGAGTGATAACACTGC | |
| | GGCCAACTTACTTCTGACAACGATCGGAGGACCGAAGGAGCTAACCGCTTTTTTGCACAAC | |
| | ATGGGGGATCATGTAACTCGCCTTGATCGTTGGGAACCGGAGCTGAATGAAGCCATACCAA | |
| | ACGACGAGCGTGACACCACGATGCCTGCAGCAATGGCAACAACGTTGCGCAAACTATTAAC | |
| | TGGCGAACTACTTACTCTAGCTTCCCGGCAACAATTAATAGACTGGATGGAGGCGGATAAA | |
| | GTTGCAGGACCACTTCTGCGCTCGGCCCTTCCGGCTAGCTGGTTTATTGCTGATAAATCTG | |
| | GAGCCGGTGAGCGTGGGTCTCGCGGTATCATTGCAGCACTGGGGCCAGATGGTAAGCCCTC | |
| | CCGTATCGTAGTTATCTACACGACGGGGAGTCAGGCAACTATGGATGAACGAAATAGACAG | |
| | ATCGCTGAGATAGGTGCCTCACTGATTAAGCATTGGTAACTGCAGACCAAGTTTACTCATA | |
| | TATACTTTAGATTGATTTAAAACTTCATTTTTAATTTAAAAGGATCTAGGTGAAGATCCTT | |
| | TTTGATAATCTCATGACCAAAATCCCTTAACGTGAGTTTTCGTTCCACTGAGCGTCAGACC | |
| | CCGTAGAAAAGATCAAAGGATCTTCTTGAGATCCTTTTTTTCTGCGCGTAATCTGCTGCTT | |
| | GCAAACAAAAAAACCACCGCTACCAGCGGTGGTTTGTTTGCCGGATCAAGAGCTACCAACT | |
| | CTTTTTCCGAAGGTAACTGGCTTCAGCAGAGCGCAGATACCAAATACTGTCCTTCTAGTGT | |
| | AGCCGTAGTTAGGCCACCACTTCAAGAACTCTGTAGCACCGCCTACATACCTCGCTCTGCT | |
| | AATCCTGTTACCAGTGGCTGCTGCCAGTGGCGATAAGTCGTGTCTTACCGGGTTGGACTCA | |
| | AGACGATAGTTACCGGATAAGGCGCAGCGGTCGGGCTGAACGGGGGGTTCGTGCACACAGC | |
| | CCAGCTTGGAGCGAACGACCTACACCGAACTGAGATACCTACAGCGTGAGCTATGAGAAAG | |
| | CGCCACGCTTCCCGAAGGGAGAAAGGCGGACAGGTATCCGGTAAGCGGCAGGGTCGGAACA | |
| | GGAGAGCGCACGAGGGAGCTTCCAGGGGGAAACGCCTGGTATCTTTATAGTCCTGTCGGGT | |
| | TTCGCCACCTCTGACTTGAGCGTCGATTTTTGTGATGCTCGTCAGGGGGGCGGAGCCTATG | |
| | GAAAAACGCCAGCAACGCGGCCTTTTTACGGTTCCTGGCCTTTTGCTGG | |
| Δh8 | GCGGCCGCGATCTCTCACCTACCAAACAATGCCCCCCTGCAAAAAATAAATTCATATAAAA | 106 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGGTTGTGCCCTTGAGGCGTGGCT | |
| | TCCGGAGCTAACGCGTTAAGTCGACCGCCTGGGGAGTACGGCCGCAAGGTTAAAACTCAAA | |
| | TGAATTGACGGGGGCCCGCACAAGCGGTGGAGCATGTGGTTTAATTCGATGCAACGCGAAG | |
| | AACCTTACCTGGTCTTGACATCCACGGAAGTTTTCAGAGATGAGAATGTGCCTTCGGGAAC | |
| | CGTGAGACAGGTGCTGCATGGCTGTCGTCAGCTCGTGTTGTGAAATGTTGGGTTAAGTCCC | |
| | GCAACGAGCGCAACCCTTATCCTTTGTTGCCAGCGGTCCGGCCGGGAACTCAAAGGAGACT | |
| | GCCAGTGATAAACTGGAGGAAGGTGGGGATGACGTCAAGTCATCATGGCCCTTACGACCAG | |
| | GGCTACACACGTGCTACAATGGCGCATACAAAGAGAAGCGACCTCGCGAGAGCAAGCGGAC | |
| | CTCATAAAGTGCGTCGTAGTCCGGATTGGAGTCTGCAACTCGACTCCATGAAGTCGGAATC | |
| | GCTAGTAATCGTGGATCAGAATGCCACGGTGAATACGTTCCCGGGCCTTGTACACACCGCC | |
| | CGTCACACCATGGGAGTGGGTTGCAAAAGAAGTAGGTAGCTTAACCTTCGGGAGGGCGCTT | |
| | ACCACTTTGTGATTCATGACTGGGGTGAAGTCGTAACAAGGTAACCGTAGGGGAACCTGCG | |
| | GTTGGATCAtgtggtTACCTTAAAGAAGCGTACTTTGTAGTGCTCACACAGATTGTCTGAT | |
| | AGAAAGTGAAAAGCAAGGCGTTTACGCGTTGGGAGTGAGGCTGAAGAGAATAAGGCCGTTC | |
| | GCTTTCTATTAATGAAAGCTCACCCTACACGAAAATATCACGCAACGCGTGATAAGCAATT | |
| | TTCGTGTCCCCTTCGTCTAGAGGCCCAGGACACCGCCCTTTCACGGCGGTAACAGGGGTTC | |
| | GAATCCCCTAGGGGACGCCACTTGCTGGTTTGTGAGTGAAAGTCGCCGACCTTAATATCTC | |
| | AAAACTCATCTTCGGGTGATGTTTGAGATATTTGCTCTTTAAAAATCTGGATCAAGCTGAA | |
| | AATTGAAACACTGAACAACGAGAGTTGTTCGTGAGTCTCTCAAATTTTCGCAACACGATGA | |
| | TGAATCGAAAGAAACATCTTCGGGTTGTGAGGTTAAGCGACTAAGCGTACACGGTGGATGC | |
| | CCTGGCAGTCAGAGGCGATGAAGGACGTGCTAATCTGCGATAAGCGTCGGTAAGGTGATAT | |
| | GAACCGTTATAACCGGCGATTTCCGAATGGGGAAACCCAGTGTGTTTCGACACACTATCAT | |
| | TAACTGAATCCATAGGTTAATGAGGCGAACCGGGGGAACTGAAACATCTAAGTACCCCGAG | |
| | GAAAAGAAATCAACCGAGATTCCCCCAGTAGCGGCGAGCGAACGGGGAGCAGCCCAGAGCC | |
| | TGAATCAGTGTGTGTGTTAGTGGAAGCGTCTGGAAAGGCGCGCGATACAGGGTGACAGCCC | |
| | CGTACACAAAAATGCACATGCTGTGAGCTCGATGAGTAGGGCGGGACACGTGGTATCCTGT | |
| | CTGAATATGGGGGGACCATCCTCCAAGGCTAAATACTCCTGACTGACCGATAGTGAACCAG | |
| | TACCGTGAGGGAAAGGCGAAAAGAACCCCGGCGAGGGGAGTGAAAAAGAACCTGAAACCGT | |
| | GTACGTACAAGCAGTGGGAGCACGCTTAGGCGTGTGACTGCGTACCTTTTGTATAATGGGT | |
| | CAGCGACTTATATTCTGTAGCAAGGTTAACCGAATAGGGGAGCCGAAGGGAAACCGAGTCT | |
| | TAACTGGGCGTTAAGTTGCAGGGTATAGACCCGAAACCCGGTGATCTAGCCATGGGCAGGT | |
| | TGAAGGTTGGGTAACACTAACTGGAGGACCGAACCGACTAATGTTGAAAAATTAGCGGATG | |
| | ACTTGTGGCTGGGGGTGAAAGGCCAATCAAACCGGGAGATAGCTGGTTCTCCCCGAAAGCT | |
| | ATTTAGGTAGCGCCTCGTGAATTCATCTCCGGGGGTAGAGCACTGTTTCGGCAAGGGGGTC | |
| | ATCCCGACTTACCAACCCGATGCAAACTGCGAATACCGGAGAATGTTATCACGGGAGACAC | |
| | ACGGCGGGTGCTAACGTCCGTCGTGAAGAGGGAAACAACCCAGACCGCCAGCTAAGGTCCC | |
| | AAAGTCATGGTTAAGTGGGAAACGATGTGGGAAGGCCCAGACAGCCAGGATGTTGGCTTAG | |
| | AAGCAGCCATCATTTAAAGAAAGCGTAATAGCTCACTGGTCGAGTCGGCCTGCGCGGAAGA | |
| | TGTAACGGGGCTAAACCATGCACCGAAGCTGCGGCAGCGACGCTTATGCGTTGTTGGGTAG | |
| | GGGAGCGTTCTGTAAGCCTGCGAAGGTGTGCTGTGAGGCATGCTGGAGGTATCAGAAGTGC | |
| | GAATGCTGACATAAGTAACGATAAAGCGGGTGAAAAGCCCGCTCGCCGGAAGACCAAGGGT | |
| | TCCTGTCCAACGTTAATCGGGGCAGGGTGAGTCGACCCCTAAGGCGAGGCCGAAAGGCGTA | |
| | GTCGATGGGAAACAGGTTAATATTCCTGTACTTGGTGTTACTGCGAAGGGGGGACGGAGAA | |
| | GGCTATGTTGGCCGGGCGACGGTTGTCCCGGTTTAAGCGTGTAGGCTGGTTTTCCAGGCAA | |
| | ATCCGGAAAATCAAGGCTGAGGCGTGATGACGAGGCACTACGGTGCTGAAGCAACAAATGC | |
| | CCTGCTTCCAGGAAAAGCCTCTAAGCATCAGGTAACATCAAATCGTACCCCAAACCGACAC | |
| | AGGTGGTCAGGTAGAGAATACCAAGGCGCTTGAGAGAACTCGGGTGAAGGAACTAGGCAAA | |
| | ATGGTGCCGTAACTTCGGGAGAAGGCACGCTGATATGTAGGTGAGGTCCCTCGCGGATGGA | |
| | GCTGAAATCAGTCGAAGATACCAGCTGGCTGCAACTGTTTATTAAAAACACAGCACTGTGC | |
| | AAACACGAAAGTGGACGTATACGGTGTGACGCCTGCCCGGTGCCGGAAGGTTAATTGATGG | |
| | GGTTAGCGCAAGCGAAGCTCTTGATCGAAGCCCCGGTAAACGGCGGCCGTAACTATAACGG | |
| | TCCTAAGGTAGCGAAATTCCTTGTCGGGTAAGTTCCGACCTGCACGAATGGCGTAATGATG | |
| | GCCAGGCTGTCTCCACCCGAGACTCAGTGAAATTGAACTCGCTGTGAAGATGCAGTGTACC | |
| | CGCGGCAAGACGGAAAGACCCCGTGAACCTTTACTATAGCTTGACACTGAACATTGAGCCT | |
| | TGATGTGTAGGATAGGTGGGAGGCTTTGAAGTGTGGACGCCAGTCTGCATGGAGCCGACCT | |
| | TGAAATACCACCCTTTAATGTTTGATGTTCTAACGTTGACCCGTAATCCGGGTTGCGGACA | |
| | GTGTCTGGTGGGTAGTTTGACTGGGGCGGTCTCCTCCTAAAGAGTAACGGAGGAGCACGAA | |
| | GGTTGGCTAATCCTGGTCGGACATCAGGAGGTTAGTGCAATGGCATAAGCCAGCTTGACTG | |
| | CGAGCGTGACGGCGCGAGCAGGTGCGAAAGCAGGTCATAGTGATCCGGTGGTTCTGAATGG | |
| | AAGGGCCATCGCTCAACGGATAAAAGGTACTCCGGGGATAACAGGCTGATACCGCCCAAGA | |
| | GTTCATATCGACGGCGGTGTTTGGCACCTCGATGTCGGCTCATCACATCCTGGGGCTGAAG | |
| | TAGGTCCCAAGGGTATGGCTGTTCGCCATTTAAAGTGGTACGCGAGCTGGGTTTAGAACGT | |
| | CGTGAGACAGTTCGGTCCCTATCTGCCGTGGGCGCTGGAGAACTGAGGGGGGCTGCTCCTA | |
| | GTACGAGAGGACCGGAGTGGACGCATCACTGGTGTTCGGGTTGTCATGCCAATGGCACTGC | |
| | CCGGTAGCTAAATGCGGAAGAGATAAGTGCTGAAAGCATCTAAGCACGAAACTTGCCCCGA | |
| | GATGAGTTCTCCCTGACCCTTTAAGGGTCCTGAAGGAACGTTGAAGACGACGACGTTGATA | |
| | GGCCGGGTGTGTAAGCGCAGCGATGCGTTGAGCTAACCGGTACTAATGAACCGTGAGGCTT | |
| | AACCTTACAACGCCGAAGCTGTTTTGGCGGATGAGAGAAGATTTTCAGCCTGATACAGATT | |
| | AAATCAGAACGCAGAAGCGGTCTGATAAAACAGAATTTGCCTGGCGGCAGTAGCGCGGTGG | |
| | TCCCACCTGACCCCATGCCGAACTCAGAAGTGAAACGCCGTAGCGCCGATGGTAGTGTGGG | |
| | GTCTCCCCATGCGAGAGTAGGGAACTGCCAGGCATCAAATAAAACGAAAGGCTCAGTCGAA | |
| | AGACTGGGCCTTTCGTTTTATCTGTTGTTTGTCGGTGAACGCTCTCCTGAGTAGGACAAAT | |
| | CCGCCGGGAGCGGATTTGAACGTTGCGAAGCAACGGCCCGGAGGGTGGCGGGCAGGACGCC | |
| | CGCCATAAACTGCCAGGCATCAAATTAAGCAGAAGGCCATCCTGACGGATGGCCTTTTTGC | |
| | GTTTCTACAAACTCTTCCTGTCGTCATATCTACAAGCCGGCGCGCCGGGAAATGTGCGCGG | |
| | AACCCCTATTTGTTTATTTTTCTAAATACATTCAAATATGTATCCGCTCATGAGACAATAA | |
| | CCCTGATAAATGCTTCAATAATATTGAAAAAGGAAGAGTATGAGTATTCAACATTTCCGTG | |
| | TCGCCCTTATTCCCTTTTTTGCGGCATTTTGCCTTCCTGTTTTTGCTCACCCAGAAACGCT | |
| | GGTGAAAGTAAAAGATGCTGAAGATCAGTTGGGTGCACGAGTGGGTTACATCGAACTGGAT | |
| | CTCAACAGCGGTAAGATCCTTGAGAGTTTTCGCCCCGAAGAACGTTTTCCAATGATGAGCA | |
| | CTTTTAAAGTTCTGCTATGTGGCGCGGTATTATCCCGTGTTGACGCCGGGCAAGAGCAACT | |
| | CGGTCGCCGCATACACTATTCTCAGAATGACTTGGTTGAGTACTCACCAGTCACAGAAAAG | |
| | CATCTTACGGATGGCATGACAGTAAGAGAATTATGCAGTGCTGCAATAACCATGAGTGATA | |
| | ACACTGCGGCCAACTTACTTCTGACAACGATCGGAGGACCGAAGGAGCTAACCGCTTTTTT | |
| | GCACAACATGGGGGATCATGTAACTCGCCTTGATCGTTGGGAACCGGAGCTGAATGAAGCC | |
| | ATACCAAACGACGAGCGTGACACCACGATGCCTGCAGCAATGGCAACAACGTTGCGCAAAC | |
| | TATTAACTGGCGAACTACTTACTCTAGCTTCCCGGCAACAATTAATAGACTGGATGGAGGC | |
| | GGATAAAGTTGCAGGACCACTTCTGCGCTCGGCCCTTCCGGCTAGCTGGTTTATTGCTGAT | |
| | AAATCTGGAGCCGGTGAGCGTGGGTCTCGCGGTATCATTGCAGCACTGGGGCCAGATGGTA | |
| | AGCCCTCCCGTATCGTAGTTATCTACACGACGGGGAGTCAGGCAACTATGGATGAACGAAA | |
| | TAGACAGATCGCTGAGATAGGTGCCTCACTGATTAAGCATTGGTAACTGCAGACCAAGTTT | |
| | ACTCATATATACTTTAGATTGATTTAAAACTTCATTTTTAATTTAAAAGGATCTAGGTGAA | |
| | GATCCTTTTTGATAATCTCATGACCAAAATCCCTTAACGTGAGTTTTCGTTCCACTGAGCG | |
| | TCAGACCCCGTAGAAAAGATCAAAGGATCTTCTTGAGATCCTTTTTTTCTGCGCGTAATCT | |
| | GCTGCTTGCAAACAAAAAAACCACCGCTACCAGCGGTGGTTTGTTTGCCGGATCAAGAGCT | |
| | ACCAACTCTTTTTCCGAAGGTAACTGGCTTCAGCAGAGCGCAGATACCAAATACTGTCCTT | |
| | CTAGTGTAGCCGTAGTTAGGCCACCACTTCAAGAACTCTGTAGCACCGCCTACATACCTCG | |
| | CTCTGCTAATCCTGTTACCAGTGGCTGCTGCCAGTGGCGATAAGTCGTGTCTTACCGGGTT | |
| | GGACTCAAGACGATAGTTACCGGATAAGGCGCAGCGGTCGGGCTGAACGGGGGGTTCGTGC | |
| | ACACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAGGGT | |
| | CGGAACAGGAGAGCGCACGAGGGAGCTTCCAGGGGGAAACGCCTGGTATCTTTATAGTCCT | |
| | GTCGGGTTTCGCCACCTCTGACTTGAGCGTCGATTTTTGTGATGCTCGTCAGGGGGGCGGA | |
| | GCCTATGGAAAAACGCCAGCAACGCGGCCTTTTTACGGTTCCTGGCCTTTTGCTGG | |
| Δh26 | GCGGCCGCGATCTCTCACCTACCAAACAATGCCCCCCTGCAAAAAATAAATTCATATAAAA | 107 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACTACTGGAAACGGTAGC | |
| | TAATACCGCATAACGTCGCAAGACCAAAGAGGGGGACCTTCGGGCCTCTTGCCATCGGATG | |
| | TGCCCAGATGGGATTAGCTAGTAGGTGGGGTAACGGCTCACCTAGGCGACGATCCCTAGCT | |
| | GGTCTGAGAGGATGACCAGCCACACTGGAACTGAGACACGGTCCAGACTCCTACGGGAGGC | |
| | AGCAGTGGGGAATATTGCACAATGGGCGCAAGCCTGATGCAGCCATGCCGCGTGTATGAAG | |
| | AAGGCCTTCGGGTTGTAAAGTACTTTCAGCGGGGAGGAAGGGAGTAAAGTTAATACCTTTG | |
| | CTCATTGACGTTACCCGCAGAAGAAGCACCGGCTAACTCCGTGCCAGCAGCCGCGGTAATA | |
| | CGGAGGGTGCAAGCGTTAATCGGAATTACTGGGCGTAAAGCGCACGCAGGCGGTTTGTTAA | |
| | GTCAGATGTGAAATCCCCGGGCTCAACCTGGGAACTGCATCTGATACTGGCAAGCTTGAGT | |
| | CTCGTAGAGGGGGGTAGAATTCCAGGTGTAGCGGTGAAATGCGTAGAGATCTGGAGGAATA | |
| | CCGGTGGCGAAGGCGGCCCCCTGGACGAAGACTGACGCTCAGGTGCGAAAGCGTGGGGAGC | |
| | AAACAGGATTAGATACCCTGGTAGTCCACGCCGTAAACGATGTCGACTTGGAGCTTGATTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTGGTCTTGACATCCACGGAAGTTTTCAGAGATGAGAATGTGCCTTCGGGAACCG | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGGTCCGGCCGGGAACTCAAAGGAGACTGC | |
| | CAGTGATAAACTGGAGGAAGGTGGGGATGACGTCAAGTCATCATGGCCCTTACGACCAGGG | |
| | CTACACACGTGCTACAATGGCGCATACAAAGAGAAGCGACCTCGCGAGAGCAAGCGGACCT | |
| | CATAAAGTGCGTCGTAGTCCGGATTGGAGTCTGCAACTCGACTCCATGAAGTCGGAATCGC | |
| | TAGTAATCGTGGATCAGAATGCCACGGTGAATACGTTCCCGGGCCTTGTACACACCGCCCG | |
| | TCACACCATGGGAGTGGGTTGCAAAAGAAGTAGGTAGCTTAACCTTCGGGAGGGCGCTTAC | |
| | CACTTTGTGATTCATGACTGGGGTGAAGTCGTAACAAGGTAACCGTAGGGGAACCTGCGGT | |
| | TGGATCAtgtggtTACCTTAAAGAAGCGTACTTTGTAGTGCTCACACAGATTGTCTGATAG | |
| | AAAGTGAAAAGCAAGGCGTTTACGCGTTGGGAGTGAGGCTGAAGAGAATAAGGCCGTTCGC | |
| | TTTCTATTAATGAAAGCTCACCCTACACGAAAATATCACGCAACGCGTGATAAGCAATTTT | |
| | CGTGTCCCCTTCGTCTAGAGGCCCAGGACACCGCCCTTTCACGGCGGTAACAGGGGTTCGA | |
| | ATCCCCTAGGGGACGCCACTTGCTGGTTTGTGAGTGAAAGTCGCCGACCTTAATATCTCAA | |
| | AACTCATCTTCGGGTGATGTTTGAGATATTTGCTCTTTAAAAATCTGGATCAAGCTGAAAA | |
| | TTGAAACACTGAACAACGAGAGTTGTTCGTGAGTCTCTCAAATTTTCGCAACACGATGATG | |
| | AATCGAAAGAAACATCTTCGGGTTGTGAGGTTAAGCGACTAAGCGTACACGGTGGATGCCC | |
| | TGGCAGTCAGAGGCGATGAAGGACGTGCTAATCTGCGATAAGCGTCGGTAAGGTGATATGA | |
| | ACCGTTATAACCGGCGATTTCCGAATGGGGAAACCCAGTGTGTTTCGACACACTATCATTA | |
| | ACTGAATCCATAGGTTAATGAGGCGAACCGGGGGAACTGAAACATCTAAGTACCCCGAGGA | |
| | AAAGAAATCAACCGAGATTCCCCCAGTAGCGGCGAGCGAACGGGGAGCAGCCCAGAGCCTG | |
| | AATCAGTGTGTGTGTTAGTGGAAGCGTCTGGAAAGGCGCGCGATACAGGGTGACAGCCCCG | |
| | TACACAAAAATGCACATGCTGTGAGCTCGATGAGTAGGGCGGGACACGTGGTATCCTGTCT | |
| | GAATATGGGGGGACCATCCTCCAAGGCTAAATACTCCTGACTGACCGATAGTGAACCAGTA | |
| | CCGTGAGGGAAAGGCGAAAAGAACCCCGGCGAGGGGAGTGAAAAAGAACCTGAAACCGTGT | |
| | ACGTACAAGCAGTGGGAGCACGCTTAGGCGTGTGACTGCGTACCTTTTGTATAATGGGTCA | |
| | GCGACTTATATTCTGTAGCAAGGTTAACCGAATAGGGGAGCCGAAGGGAAACCGAGTCTTA | |
| | ACTGGGCGTTAAGTTGCAGGGTATAGACCCGAAACCCGGTGATCTAGCCATGGGCAGGTTG | |
| | AAGGTTGGGTAACACTAACTGGAGGACCGAACCGACTAATGTTGAAAAATTAGCGGATGAC | |
| | TTGTGGCTGGGGGTGAAAGGCCAATCAAACCGGGAGATAGCTGGTTCTCCCCGAAAGCTAT | |
| | TTAGGTAGCGCCTCGTGAATTCATCTCCGGGGGTAGAGCACTGTTTCGGCAAGGGGGTCAT | |
| | CCCGACTTACCAACCCGATGCAAACTGCGAATACCGGAGAATGTTATCACGGGAGACACAC | |
| | GGCGGGTGCTAACGTCCGTCGTGAAGAGGGAAACAACCCAGACCGCCAGCTAAGGTCCCAA | |
| | AGTCATGGTTAAGTGGGAAACGATGTGGGAAGGCCCAGACAGCCAGGATGTTGGCTTAGAA | |
| | GCAGCCATCATTTAAAGAAAGCGTAATAGCTCACTGGTCGAGTCGGCCTGCGCGGAAGATG | |
| | TAACGGGGCTAAACCATGCACCGAAGCTGCGGCAGCGACGCTTATGCGTTGTTGGGTAGGG | |
| | GAGCGTTCTGTAAGCCTGCGAAGGTGTGCTGTGAGGCATGCTGGAGGTATCAGAAGTGCGA | |
| | ATGCTGACATAAGTAACGATAAAGCGGGTGAAAAGCCCGCTCGCCGGAAGACCAAGGGTTC | |
| | CTGTCCAACGTTAATCGGGGCAGGGTGAGTCGACCCCTAAGGCGAGGCCGAAAGGCGTAGT | |
| | CGATGGGAAACAGGTTAATATTCCTGTACTTGGTGTTACTGCGAAGGGGGGACGGAGAAGG | |
| | CTATGTTGGCCGGGCGACGGTTGTCCCGGTTTAAGCGTGTAGGCTGGTTTTCCAGGCAAAT | |
| | CCGGAAAATCAAGGCTGAGGCGTGATGACGAGGCACTACGGTGCTGAAGCAACAAATGCCC | |
| | TGCTTCCAGGAAAAGCCTCTAAGCATCAGGTAACATCAAATCGTACCCCAAACCGACACAG | |
| | GTGGTCAGGTAGAGAATACCAAGGCGCTTGAGAGAACTCGGGTGAAGGAACTAGGCAAAAT | |
| | GGTGCCGTAACTTCGGGAGAAGGCACGCTGATATGTAGGTGAGGTCCCTCGCGGATGGAGC | |
| | TGAAATCAGTCGAAGATACCAGCTGGCTGCAACTGTTTATTAAAAACACAGCACTGTGCAA | |
| | ACACGAAAGTGGACGTATACGGTGTGACGCCTGCCCGGTGCCGGAAGGTTAATTGATGGGG | |
| | TTAGCGCAAGCGAAGCTCTTGATCGAAGCCCCGGTAAACGGCGGCCGTAACTATAACGGTC | |
| | CTAAGGTAGCGAAATTCCTTGTCGGGTAAGTTCCGACCTGCACGAATGGCGTAATGATGGC | |
| | CAGGCTGTCTCCACCCGAGACTCAGTGAAATTGAACTCGCTGTGAAGATGCAGTGTACCCG | |
| | CGGCAAGACGGAAAGACCCCGTGAACCTTTACTATAGCTTGACACTGAACATTGAGCCTTG | |
| | ATGTGTAGGATAGGTGGGAGGCTTTGAAGTGTGGACGCCAGTCTGCATGGAGCCGACCTTG | |
| | AAATACCACCCTTTAATGTTTGATGTTCTAACGTTGACCCGTAATCCGGGTTGCGGACAGT | |
| | GTCTGGTGGGTAGTTTGACTGGGGCGGTCTCCTCCTAAAGAGTAACGGAGGAGCACGAAGG | |
| | TTGGCTAATCCTGGTCGGACATCAGGAGGTTAGTGCAATGGCATAAGCCAGCTTGACTGCG | |
| | AGCGTGACGGCGCGAGCAGGTGCGAAAGCAGGTCATAGTGATCCGGTGGTTCTGAATGGAA | |
| | GGGCCATCGCTCAACGGATAAAAGGTACTCCGGGGATAACAGGCTGATACCGCCCAAGAGT | |
| | TCATATCGACGGCGGTGTTTGGCACCTCGATGTCGGCTCATCACATCCTGGGGCTGAAGTA | |
| | GGTCCCAAGGGTATGGCTGTTCGCCATTTAAAGTGGTACGCGAGCTGGGTTTAGAACGTCG | |
| | TGAGACAGTTCGGTCCCTATCTGCCGTGGGCGCTGGAGAACTGAGGGGGGCTGCTCCTAGT | |
| | ACGAGAGGACCGGAGTGGACGCATCACTGGTGTTCGGGTTGTCATGCCAATGGCACTGCCC | |
| | GGTAGCTAAATGCGGAAGAGATAAGTGCTGAAAGCATCTAAGCACGAAACTTGCCCCGAGA | |
| | TGAGTTCTCCCTGACCCTTTAAGGGTCCTGAAGGAACGTTGAAGACGACGACGTTGATAGG | |
| | CCGGGTGTGTAAGCGCAGCGATGCGTTGAGCTAACCGGTACTAATGAACCGTGAGGCTTAA | |
| | CCTTACAACGCCGAAGCTGTTTTGGCGGATGAGAGAAGATTTTCAGCCTGATACAGATTAA | |
| | ATCAGAACGCAGAAGCGGTCTGATAAAACAGAATTTGCCTGGCGGCAGTAGCGCGGTGGTC | |
| | CCACCTGACCCCATGCCGAACTCAGAAGTGAAACGCCGTAGCGCCGATGGTAGTGTGGGGT | |
| | CTCCCCATGCGAGAGTAGGGAACTGCCAGGCATCAAATAAAACGAAAGGCTCAGTCGAAAG | |
| | ACTGGGCCTTTCGTTTTATCTGTTGTTTGTCGGTGAACGCTCTCCTGAGTAGGACAAATCC | |
| | GCCGGGAGCGGATTTGAACGTTGCGAAGCAACGGCCCGGAGGGTGGCGGGCAGGACGCCCG | |
| | CCATAAACTGCCAGGCATCAAATTAAGCAGAAGGCCATCCTGACGGATGGCCTTTTTGCGT | |
| | TTCTACAAACTCTTCCTGTCGTCATATCTACAAGCCGGCGCGCCGGGAAATGTGCGCGGAA | |
| | CCCCTATTTGTTTATTTTTCTAAATACATTCAAATATGTATCCGCTCATGAGACAATAACC | |
| | CTGATAAATGCTTCAATAATATTGAAAAAGGAAGAGTATGAGTATTCAACATTTCCGTGTC | |
| | GCCCTTATTCCCTTTTTTGCGGCATTTTGCCTTCCTGTTTTTGCTCACCCAGAAACGCTGG | |
| | TGAAAGTAAAAGATGCTGAAGATCAGTTGGGTGCACGAGTGGGTTACATCGAACTGGATCT | |
| | CAACAGCGGTAAGATCCTTGAGAGTTTTCGCCCCGAAGAACGTTTTCCAATGATGAGCACT | |
| | TTTAAAGTTCTGCTATGTGGCGCGGTATTATCCCGTGTTGACGCCGGGCAAGAGCAACTCG | |
| | GTCGCCGCATACACTATTCTCAGAATGACTTGGTTGAGTACTCACCAGTCACAGAAAAGCA | |
| | TCTTACGGATGGCATGACAGTAAGAGAATTATGCAGTGCTGCAATAACCATGAGTGATAAC | |
| | ACTGCGGCCAACTTACTTCTGACAACGATCGGAGGACCGAAGGAGCTAACCGCTTTTTTGC | |
| | ACAACATGGGGGATCATGTAACTCGCCTTGATCGTTGGGAACCGGAGCTGAATGAAGCCAT | |
| | ACCAAACGACGAGCGTGACACCACGATGCCTGCAGCAATGGCAACAACGTTGCGCAAACTA | |
| | TTAACTGGCGAACTACTTACTCTAGCTTCCCGGCAACAATTAATAGACTGGATGGAGGCGG | |
| | ATAAAGTTGCAGGACCACTTCTGCGCTCGGCCCTTCCGGCTAGCTGGTTTATTGCTGATAA | |
| | ATCTGGAGCCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGAACGAAATA | |
| | GACAGATCGCTGAGATAGGTGCCTCACTGATTAAGCATTGGTAACTGCAGACCAAGTTTAC | |
| | TCATATATACTTTAGATTGATTTAAAACTTCATTTTTAATTTAAAAGGATCTAGGTGAAGA | |
| | TCCTTTTTGATAATCTCATGACCAAAATCCCTTAACGTGAGTTTTCGTTCCACTGAGCGTC | |
| | AGACCCCGTAGAAAAGATCAAAGGATCTTCTTGAGATCCTTTTTTTCTGCGCGTAATCTGC | |
| | TGCTTGCAAACAAAAAAACCACCGCTACCAGCGGTGGTTTGTTTGCCGGATCAAGAGCTAC | |
| | CAACTCTTTTTCCGAAGGTAACTGGCTTCAGCAGAGCGCAGATACCAAATACTGTCCTTCT | |
| | AGTGTAGCCGTAGTTAGGCCACCACTTCAAGAACTCTGTAGCACCGCCTACATACCTCGCT | |
| | CTGCTAATCCTGTTACCAGTGGCTGCTGCCAGTGGCGATAAGTCGTGTCTTACCGGGTTGG | |
| | ACTCAAGACGATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGTGAGCTATGA | |
| | GAAAGCGCCACGCTTCCCGAAGGGAGAAAGGCGGACAGGTATCCGGTAAGCGGCAGGGTCG | |
| | GAACAGGAGAGCGCACGAGGGAGCTTCCAGGGGGAAACGCCTGGTATCTTTATAGTCCTGT | |
| | CGGGTTTCGCCACCTCTGACTTGAGCGTCGATTTTTGTGATGCTCGTCAGGGGGGCGGAGC | |
| | CTATGGAAAAACGCCAGCAACGCGGCCTTTTTACGGTTCCTGGCCTTTTGCTGG | |
| Δh33 | GCGGCCGCGATCTCTCACCTACCAAACAATGCCCCCCTGCAAAAAATAAATTCATATAAAA | 108 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACTACTGGAAACGGTAGC | |
| | TAATACCGCATAACGTCGCAAGACCAAAGAGGGGGACCTTCGGGCCTCTTGCCATCGGATG | |
| | TGCCCAGATGGGATTAGCTAGTAGGTGGGGTAACGGCTCACCTAGGCGACGATCCCTAGCT | |
| | GGTCTGAGAGGATGACCAGCCACACTGGAACTGAGACACGGTCCAGACTCCTACGGGAGGC | |
| | AGCAGTGGGGAATATTGCACAATGGGCGCAAGCCTGATGCAGCCATGCCGCGTGTATGAAG | |
| | AAGGCCTTCGGGTTGTAAAGTACTTTCAGCGGGGAGGAAGGGAGTAAAGTTAATACCTTTG | |
| | CTCATTGACGTTACCCGCAGAAGAAGCACCGGCTAACTCCGTGCCAGCAGCCGCGGTAATA | |
| | CGGAGGGTGCAAGCGTTAATCGGAATTACTGGGCGTAAAGCGCACGCAGGCGGTTTGTTAA | |
| | GTCAGATGTGAAATCCCCGGGCTCAACCTGGGAACTGCATCTGATACTGGCAAGCTTGAGT | |
| | CTCGTAGAGGGGGGTAGAATTCCAGGTGTAGCGGTGAAATGCGTAGAGATCTGGAGGAATA | |
| | CCGGTGGCGAAGGCGGCCCCCTGGACGAAGACTGACGCTCAGGTGCGAAAGCGTGGGGAGC | |
| | AAACAGGATTAGATACCCTGGTAGTCCACGCCGTAAACGATGTCGACTTGGAGGTTGTGCC | |
| | CTTGAGGCGTGGCTTCCGGAGCTAACGCGTTAAGTCGACCGCCTGGGGAGTACGGCCGCAA | |
| | GGTTAAAACTCAAATGAATTGACGGGGGCCCGCACAAGCGGTGGAGCATGTGGTTTAATTC | |
| | GATGCAACGCGAAGAACCTTACCTGGTCTTGACATCCGAGACAGGTGCTGCATGGCTGTCG | |
| | TCAGCTCGTGTTGTGAAATGTTGGGTTAAGTCCCGCAACGAGCGCAACCCTTATCCTTTGT | |
| | TGCCAGCGGTCCGGCCGGGAACTCAAAGGAGACTGCCAGTGATAAACTGGAGGAAGGTGGG | |
| | GATGACGTCAAGTCATCATGGCCCTTACGACCAGGGCTACACACGTGCTACAATGGCGCAT | |
| | ACAAAGAGAAGCGACCTCGCGAGAGCAAGCGGACCTCATAAAGTGCGTCGTAGTCCGGATT | |
| | GGAGTCTGCAACTCGACTCCATGAAGTCGGAATCGCTAGTAATCGTGGATCAGAATGCCAC | |
| | GGTGAATACGTTCCCGGGCCTTGTACACACCGCCCGTCACACCATGGGAGTGGGTTGCAAA | |
| | AGAAGTAGGTAGCTTAACCTTCGGGAGGGCGCTTACCACTTTGTGATTCATGACTGGGGTG | |
| | AAGTCGTAACAAGGTAACCGTAGGGGAACCTGCGGTTGGATCAtgtggtTACCTTAAAGAA | |
| | GCGTACTTTGTAGTGCTCACACAGATTGTCTGATAGAAAGTGAAAAGCAAGGCGTTTACGC | |
| | GTTGGGAGTGAGGCTGAAGAGAATAAGGCCGTTCGCTTTCTATTAATGAAAGCTCACCCTA | |
| | CACGAAAATATCACGCAACGCGTGATAAGCAATTTTCGTGTCCCCTTCGTCTAGAGGCCCA | |
| | GGACACCGCCCTTTCACGGCGGTAACAGGGGTTCGAATCCCCTAGGGGACGCCACTTGCTG | |
| | GTTTGTGAGTGAAAGTCGCCGACCTTAATATCTCAAAACTCATCTTCGGGTGATGTTTGAG | |
| | ATATTTGCTCTTTAAAAATCTGGATCAAGCTGAAAATTGAAACACTGAACAACGAGAGTTG | |
| | TTCGTGAGTCTCTCAAATTTTCGCAACACGATGATGAATCGAAAGAAACATCTTCGGGTTG | |
| | TGAGGTTAAGCGACTAAGCGTACACGGTGGATGCCCTGGCAGTCAGAGGCGATGAAGGACG | |
| | TGCTAATCTGCGATAAGCGTCGGTAAGGTGATATGAACCGTTATAACCGGCGATTTCCGAA | |
| | TGGGGAAACCCAGTGTGTTTCGACACACTATCATTAACTGAATCCATAGGTTAATGAGGCG | |
| | AACCGGGGGAACTGAAACATCTAAGTACCCCGAGGAAAAGAAATCAACCGAGATTCCCCCA | |
| | GTAGCGGCGAGCGAACGGGGAGCAGCCCAGAGCCTGAATCAGTGTGTGTGTTAGTGGAAGC | |
| | GTCTGGAAAGGCGCGCGATACAGGGTGACAGCCCCGTACACAAAAATGCACATGCTGTGAG | |
| | CTCGATGAGTAGGGCGGGACACGTGGTATCCTGTCTGAATATGGGGGGACCATCCTCCAAG | |
| | GCTAAATACTCCTGACTGACCGATAGTGAACCAGTACCGTGAGGGAAAGGCGAAAAGAACC | |
| | CCGGCGAGGGGAGTGAAAAAGAACCTGAAACCGTGTACGTACAAGCAGTGGGAGCACGCTT | |
| | AGGCGTGTGACTGCGTACCTTTTGTATAATGGGTCAGCGACTTATATTCTGTAGCAAGGTT | |
| | AACCGAATAGGGGAGCCGAAGGGAAACCGAGTCTTAACTGGGCGTTAAGTTGCAGGGTATA | |
| | GACCCGAAACCCGGTGATCTAGCCATGGGCAGGTTGAAGGTTGGGTAACACTAACTGGAGG | |
| | ACCGAACCGACTAATGTTGAAAAATTAGCGGATGACTTGTGGCTGGGGGTGAAAGGCCAAT | |
| | CAAACCGGGAGATAGCTGGTTCTCCCCGAAAGCTATTTAGGTAGCGCCTCGTGAATTCATC | |
| | TCCGGGGGTAGAGCACTGTTTCGGCAAGGGGGTCATCCCGACTTACCAACCCGATGCAAAC | |
| | TGCGAATACCGGAGAATGTTATCACGGGAGACACACGGCGGGTGCTAACGTCCGTCGTGAA | |
| | GAGGGAAACAACCCAGACCGCCAGCTAAGGTCCCAAAGTCATGGTTAAGTGGGAAACGATG | |
| | TGGGAAGGCCCAGACAGCCAGGATGTTGGCTTAGAAGCAGCCATCATTTAAAGAAAGCGTA | |
| | ATAGCTCACTGGTCGAGTCGGCCTGCGCGGAAGATGTAACGGGGCTAAACCATGCACCGAA | |
| | GCTGCGGCAGCGACGCTTATGCGTTGTTGGGTAGGGGAGCGTTCTGTAAGCCTGCGAAGGT | |
| | GTGCTGTGAGGCATGCTGGAGGTATCAGAAGTGCGAATGCTGACATAAGTAACGATAAAGC | |
| | GGGTGAAAAGCCCGCTCGCCGGAAGACCAAGGGTTCCTGTCCAACGTTAATCGGGGCAGGG | |
| | TGAGTCGACCCCTAAGGCGAGGCCGAAAGGCGTAGTCGATGGGAAACAGGTTAATATTCCT | |
| | GTACTTGGTGTTACTGCGAAGGGGGGACGGAGAAGGCTATGTTGGCCGGGCGACGGTTGTC | |
| | CCGGTTTAAGCGTGTAGGCTGGTTTTCCAGGCAAATCCGGAAAATCAAGGCTGAGGCGTGA | |
| | TGACGAGGCACTACGGTGCTGAAGCAACAAATGCCCTGCTTCCAGGAAAAGCCTCTAAGCA | |
| | TCAGGTAACATCAAATCGTACCCCAAACCGACACAGGTGGTCAGGTAGAGAATACCAAGGC | |
| | GCTTGAGAGAACTCGGGTGAAGGAACTAGGCAAAATGGTGCCGTAACTTCGGGAGAAGGCA | |
| | CGCTGATATGTAGGTGAGGTCCCTCGCGGATGGAGCTGAAATCAGTCGAAGATACCAGCTG | |
| | GCTGCAACTGTTTATTAAAAACACAGCACTGTGCAAACACGAAAGTGGACGTATACGGTGT | |
| | GACGCCTGCCCGGTGCCGGAAGGTTAATTGATGGGGTTAGCGCAAGCGAAGCTCTTGATCG | |
| | AAGCCCCGGTAAACGGCGGCCGTAACTATAACGGTCCTAAGGTAGCGAAATTCCTTGTCGG | |
| | GTAAGTTCCGACCTGCACGAATGGCGTAATGATGGCCAGGCTGTCTCCACCCGAGACTCAG | |
| | TGAAATTGAACTCGCTGTGAAGATGCAGTGTACCCGCGGCAAGACGGAAAGACCCCGTGAA | |
| | CCTTTACTATAGCTTGACACTGAACATTGAGCCTTGATGTGTAGGATAGGTGGGAGGCTTT | |
| | GAAGTGTGGACGCCAGTCTGCATGGAGCCGACCTTGAAATACCACCCTTTAATGTTTGATG | |
| | TTCTAACGTTGACCCGTAATCCGGGTTGCGGACAGTGTCTGGTGGGTAGTTTGACTGGGGC | |
| | GGTCTCCTCCTAAAGAGTAACGGAGGAGCACGAAGGTTGGCTAATCCTGGTCGGACATCAG | |
| | GAGGTTAGTGCAATGGCATAAGCCAGCTTGACTGCGAGCGTGACGGCGCGAGCAGGTGCGA | |
| | AAGCAGGTCATAGTGATCCGGTGGTTCTGAATGGAAGGGCCATCGCTCAACGGATAAAAGG | |
| | TACTCCGGGGATAACAGGCTGATACCGCCCAAGAGTTCATATCGACGGCGGTGTTTGGCAC | |
| | CTCGATGTCGGCTCATCACATCCTGGGGCTGAAGTAGGTCCCAAGGGTATGGCTGTTCGCC | |
| | ATTTAAAGTGGTACGCGAGCTGGGTTTAGAACGTCGTGAGACAGTTCGGTCCCTATCTGCC | |
| | GTGGGCGCTGGAGAACTGAGGGGGGCTGCTCCTAGTACGAGAGGACCGGAGTGGACGCATC | |
| | ACTGGTGTTCGGGTTGTCATGCCAATGGCACTGCCCGGTAGCTAAATGCGGAAGAGATAAG | |
| | TGCTGAAAGCATCTAAGCACGAAACTTGCCCCGAGATGAGTTCTCCCTGACCCTTTAAGGG | |
| | TCCTGAAGGAACGTTGAAGACGACGACGTTGATAGGCCGGGTGTGTAAGCGCAGCGATGCG | |
| | TTGAGCTAACCGGTACTAATGAACCGTGAGGCTTAACCTTACAACGCCGAAGCTGTTTTGG | |
| | CGGATGAGAGAAGATTTTCAGCCTGATACAGATTAAATCAGAACGCAGAAGCGGTCTGATA | |
| | AAACAGAATTTGCCTGGCGGCAGTAGCGCGGTGGTCCCACCTGACCCCATGCCGAACTCAG | |
| | AAGTGAAACGCCGTAGCGCCGATGGTAGTGTGGGGTCTCCCCATGCGAGAGTAGGGAACTG | |
| | CCAGGCATCAAATAAAACGAAAGGCTCAGTCGAAAGACTGGGCCTTTCGTTTTATCTGTTG | |
| | TTTGTCGGTGAACGCTCTCCTGAGTAGGACAAATCCGCCGGGAGCGGATTTGAACGTTGCG | |
| | AAGCAACGGCCCGGAGGGTGGCGGGCAGGACGCCCGCCATAAACTGCCAGGCATCAAATTA | |
| | AGCAGAAGGCCATCCTGACGGATGGCCTTTTTGCGTTTCTACAAACTCTTCCTGTCGTCAT | |
| | ATCTACAAGCCGGCGCGCCGGGAAATGTGCGCGGAACCCCTATTTGTTTATTTTTCTAAAT | |
| | ACATTCAAATATGTATCCGCTCATGAGACAATAACCCTGATAAATGCTTCAATAATATTGA | |
| | AAAAGGAAGAGTATGAGTATTCAACATTTCCGTGTCGCCCTTATTCCCTTTTTTGCGGCAT | |
| | TTTGCCTTCCTGTTTTTGCTCACCCAGAAACGCTGGTGAAAGTAAAAGATGCTGAAGATCA | |
| | GTTGGGTGCACGAGTGGGTTACATCGAACTGGATCTCAACAGCGGTAAGATCCTTGAGAGT | |
| | TTTCGCCCCGAAGAACGTTTTCCAATGATGAGCACTTTTAAAGTTCTGCTATGTGGCGCGG | |
| | TATTATCCCGTGTTGACGCCGGGCAAGAGCAACTCGGTCGCCGCATACACTATTCTCAGAA | |
| | TGACTTGGTTGAGTACTCACCAGTCACAGAAAAGCATCTTACGGATGGCATGACAGTAAGA | |
| | GAATTATGCAGTGCTGCAATAACCATGAGTGATAACACTGCGGCCAACTTACTTCTGACAA | |
| | CGATCGGAGGACCGAAGGAGCTAACCGCTTTTTTGCACAACATGGGGGATCATGTAACTCG | |
| | CCTTGATCGTTGGGAACCGGAGCTGAATGAAGCCATACCAAACGACGAGCGTGACACCACG | |
| | ATGCCTGCAGCAATGGCAACAACGTTGCGCAAACTATTAACTGGCGAACTACTTACTCTAG | |
| | CTTCCCGGCAACAATTAATAGACTGGATGGAGGCGGATAAAGTTGCAGGACCACTTCTGCG | |
| | CTCGGCCCTTCCGGCTAGCTGGTTTATTGCTGATAAATCTGGAGCCGGTGAGCGTGGGTCT | |
| | CGCGGTATCATTGCAGCACTGGGGCCAGATGGTAAGCCCTCCCGTATCGTAGTTATCTACA | |
| | CGACGGGGAGTCAGGCAACTATGGATGAACGAAATAGACAGATCGCTGAGATAGGTGCCTC | |
| | ACTGATTAAGCATTGGTAACTGCAGACCAAGTTTACTCATATATACTTTAGATTGATTTAA | |
| | AACTTCATTTTTAATTTAAAAGGATCTAGGTGAAGATCCTTTTTGATAATCTCATGACCAA | |
| | AATCCCTTAACGTGAGTTTTCGTTCCACTGAGCGTCAGACCCCGTAGAAAAGATCAAAGGA | |
| | TCTTCTTGAGATCCTTTTTTTCTGCGCGTAATCTGCTGCTTGCAAACAAAAAAACCACCGC | |
| | TACCAGCGGTGGTTTGTTTGCCGGATCAAGAGCTACCAACTCTTTTTCCGAAGGTAACTGG | |
| | CTTCAGCAGAGCGCAGATACCAAATACTGTCCTTCTAGTGTAGCCGTAGTTAGGCCACCAC | |
| | TTCAAGAACTCTGTAGCACCGCCTACATACCTCGCTCTGCTAATCCTGTTACCAGTGGCTG | |
| | CTGCCAGTGGCGATAAGTCGTGTCTTACCGGGTTGGACTCAAGACGATAGTTACCGGATAA | |
| | GGCGCAGCGGTCGGGCTGAACGGGGGGTTCGTGCACACAGCCCAGCTTGGAGCGAACGACC | |
| | TACACCGAACTGAGATACCTACAGCGTGAGCTATGAGAAAGCGCCACGCTTCCCGAAGGGA | |
| | GAAAGGCGGACAGGTATCCGGTAAGCGGCAGGGTCGGAACAGGAGAGCGCACGAGGGAGCT | |
| | TCCAGGGGGAAACGCCTGGTATCTTTATAGTCCTGTCGGGTTTCGCCACCTCTGACTTGAG | |
| | CGTCGATTTTTGTGATGCTCGTCAGGGGGGCGGAGCCTATGGAAAAACGCCAGCAACGCGG | |
| | CCTTTTTACGGTTCCTGGCCTTTTGCTGG | |
| Δh41 | GCGGCCGCGATCTCTCACCTACCAAACAATGCCCCCCTGCAAAAAATAAATTCATATAAAA | 109 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACTACTGGAAACGGTAGC | |
| | TAATACCGCATAACGTCGCAAGACCAAAGAGGGGGACCTTCGGGCCTCTTGCCATCGGATG | |
| | TGCCCAGATGGGATTAGCTAGTAGGTGGGGTAACGGCTCACCTAGGCGACGATCCCTAGCT | |
| | GGTCTGAGAGGATGACCAGCCACACTGGAACTGAGACACGGTCCAGACTCCTACGGGAGGC | |
| | AGCAGTGGGGAATATTGCACAATGGGCGCAAGCCTGATGCAGCCATGCCGCGTGTATGAAG | |
| | AAGGCCTTCGGGTTGTAAAGTACTTTCAGCGGGGAGGAAGGGAGTAAAGTTAATACCTTTG | |
| | CTCATTGACGTTACCCGCAGAAGAAGCACCGGCTAACTCCGTGCCAGCAGCCGCGGTAATA | |
| | CGGAGGGTGCAAGCGTTAATCGGAATTACTGGGCGTAAAGCGCACGCAGGCGGTTTGTTAA | |
| | GTCAGATGTGAAATCCCCGGGCTCAACCTGGGAACTGCATCTGATACTGGCAAGCTTGAGT | |
| | CTCGTAGAGGGGGGTAGAATTCCAGGTGTAGCGGTGAAATGCGTAGAGATCTGGAGGAATA | |
| | CCGGTGGCGAAGGCGGCCCCCTGGACGAAGACTGACGCTCAGGTGCGAAAGCGTGGGGAGC | |
| | AAACAGGATTAGATACCCTGGTAGTCCACGCCGTAAACGATGTCGACTTGGAGGTTGTGCC | |
| | CTTGAGGCGTGGCTTCCGGAGCTAACGCGTTAAGTCGACCGCCTGGGGAGTACGGCCGCAA | |
| | GGTTAAAACTCAAATGAATTGACGGGGGCCCGCACAAGCGGTGGAGCATGTGGTTTAATTC | |
| | GATGCAACGCGAAGAACCTTACCTGGTCTTGACATCCACGGAAGTTTTCAGAGATGAGAAT | |
| | GTGCCTTCGGGAACCGTGAGACAGGTGCTGCATGGCTGTCGTCAGCTCGTGTTGTGAAATG | |
| | TTGGGTTAAGTCCCGCAACGAGCGCAACCCTTATCCTTTGTTGCCAGCGGTCCGGCCGGGA | |
| | ACTCAAAGGAGACTGCCAGTGATAAACTGGAGGAAGGTGGGGATGACGTCAAGTCATCATG | |
| | GCCCTTACGACCAGGGCTACACACGTGCTACAATGGCGCATACAAAGAGAGACCTCATAAA | |
| | GTGCGTCGTAGTCCGGATTGGAGTCTGCAACTCGACTCCATGAAGTCGGAATCGCTAGTAA | |
| | TCGTGGATCAGAATGCCACGGTGAATACGTTCCCGGGCCTTGTACACACCGCCCGTCACAC | |
| | CATGGGAGTGGGTTGCAAAAGAAGTAGGTAGCTTAACCTTCGGGAGGGCGCTTACCACTTT | |
| | GTGATTCATGACTGGGGTGAAGTCGTAACAAGGTAACCGTAGGGGAACCTGCGGTTGGATC | |
| | AtgtggtTACCTTAAAGAAGCGTACTTTGTAGTGCTCACACAGATTGTCTGATAGAAAGTG | |
| | AAAAGCAAGGCGTTTACGCGTTGGGAGTGAGGCTGAAGAGAATAAGGCCGTTCGCTTTCTA | |
| | TTAATGAAAGCTCACCCTACACGAAAATATCACGCAACGCGTGATAAGCAATTTTCGTGTC | |
| | CCCTTCGTCTAGAGGCCCAGGACACCGCCCTTTCACGGCGGTAACAGGGGTTCGAATCCCC | |
| | TAGGGGACGCCACTTGCTGGTTTGTGAGTGAAAGTCGCCGACCTTAATATCTCAAAACTCA | |
| | TCTTCGGGTGATGTTTGAGATATTTGCTCTTTAAAAATCTGGATCAAGCTGAAAATTGAAA | |
| | CACTGAACAACGAGAGTTGTTCGTGAGTCTCTCAAATTTTCGCAACACGATGATGAATCGA | |
| | AAGAAACATCTTCGGGTTGTGAGGTTAAGCGACTAAGCGTACACGGTGGATGCCCTGGCAG | |
| | TCAGAGGCGATGAAGGACGTGCTAATCTGCGATAAGCGTCGGTAAGGTGATATGAACCGTT | |
| | ATAACCGGCGATTTCCGAATGGGGAAACCCAGTGTGTTTCGACACACTATCATTAACTGAA | |
| | TCCATAGGTTAATGAGGCGAACCGGGGGAACTGAAACATCTAAGTACCCCGAGGAAAAGAA | |
| | ATCAACCGAGATTCCCCCAGTAGCGGCGAGCGAACGGGGAGCAGCCCAGAGCCTGAATCAG | |
| | TGTGTGTGTTAGTGGAAGCGTCTGGAAAGGCGCGCGATACAGGGTGACAGCCCCGTACACA | |
| | AAAATGCACATGCTGTGAGCTCGATGAGTAGGGCGGGACACGTGGTATCCTGTCTGAATAT | |
| | GGGGGGACCATCCTCCAAGGCTAAATACTCCTGACTGACCGATAGTGAACCAGTACCGTGA | |
| | GGGAAAGGCGAAAAGAACCCCGGCGAGGGGAGTGAAAAAGAACCTGAAACCGTGTACGTAC | |
| | AAGCAGTGGGAGCACGCTTAGGCGTGTGACTGCGTACCTTTTGTATAATGGGTCAGCGACT | |
| | TATATTCTGTAGCAAGGTTAACCGAATAGGGGAGCCGAAGGGAAACCGAGTCTTAACTGGG | |
| | CGTTAAGTTGCAGGGTATAGACCCGAAACCCGGTGATCTAGCCATGGGCAGGTTGAAGGTT | |
| | GGGTAACACTAACTGGAGGACCGAACCGACTAATGTTGAAAAATTAGCGGATGACTTGTGG | |
| | CTGGGGGTGAAAGGCCAATCAAACCGGGAGATAGCTGGTTCTCCCCGAAAGCTATTTAGGT | |
| | AGCGCCTCGTGAATTCATCTCCGGGGGTAGAGCACTGTTTCGGCAAGGGGGTCATCCCGAC | |
| | TTACCAACCCGATGCAAACTGCGAATACCGGAGAATGTTATCACGGGAGACACACGGCGGG | |
| | TGCTAACGTCCGTCGTGAAGAGGGAAACAACCCAGACCGCCAGCTAAGGTCCCAAAGTCAT | |
| | GGTTAAGTGGGAAACGATGTGGGAAGGCCCAGACAGCCAGGATGTTGGCTTAGAAGCAGCC | |
| | ATCATTTAAAGAAAGCGTAATAGCTCACTGGTCGAGTCGGCCTGCGCGGAAGATGTAACGG | |
| | GGCTAAACCATGCACCGAAGCTGCGGCAGCGACGCTTATGCGTTGTTGGGTAGGGGAGCGT | |
| | TCTGTAAGCCTGCGAAGGTGTGCTGTGAGGCATGCTGGAGGTATCAGAAGTGCGAATGCTG | |
| | ACATAAGTAACGATAAAGCGGGTGAAAAGCCCGCTCGCCGGAAGACCAAGGGTTCCTGTCC | |
| | AACGTTAATCGGGGCAGGGTGAGTCGACCCCTAAGGCGAGGCCGAAAGGCGTAGTCGATGG | |
| | GAAACAGGTTAATATTCCTGTACTTGGTGTTACTGCGAAGGGGGGACGGAGAAGGCTATGT | |
| | TGGCCGGGCGACGGTTGTCCCGGTTTAAGCGTGTAGGCTGGTTTTCCAGGCAAATCCGGAA | |
| | AATCAAGGCTGAGGCGTGATGACGAGGCACTACGGTGCTGAAGCAACAAATGCCCTGCTTC | |
| | CAGGAAAAGCCTCTAAGCATCAGGTAACATCAAATCGTACCCCAAACCGACACAGGTGGTC | |
| | AGGTAGAGAATACCAAGGCGCTTGAGAGAACTCGGGTGAAGGAACTAGGCAAAATGGTGCC | |
| | GTAACTTCGGGAGAAGGCACGCTGATATGTAGGTGAGGTCCCTCGCGGATGGAGCTGAAAT | |
| | CAGTCGAAGATACCAGCTGGCTGCAACTGTTTATTAAAAACACAGCACTGTGCAAACACGA | |
| | AAGTGGACGTATACGGTGTGACGCCTGCCCGGTGCCGGAAGGTTAATTGATGGGGTTAGCG | |
| | CAAGCGAAGCTCTTGATCGAAGCCCCGGTAAACGGCGGCCGTAACTATAACGGTCCTAAGG | |
| | TAGCGAAATTCCTTGTCGGGTAAGTTCCGACCTGCACGAATGGCGTAATGATGGCCAGGCT | |
| | GTCTCCACCCGAGACTCAGTGAAATTGAACTCGCTGTGAAGATGCAGTGTACCCGCGGCAA | |
| | GACGGAAAGACCCCGTGAACCTTTACTATAGCTTGACACTGAACATTGAGCCTTGATGTGT | |
| | AGGATAGGTGGGAGGCTTTGAAGTGTGGACGCCAGTCTGCATGGAGCCGACCTTGAAATAC | |
| | CACCCTTTAATGTTTGATGTTCTAACGTTGACCCGTAATCCGGGTTGCGGACAGTGTCTGG | |
| | TGGGTAGTTTGACTGGGGCGGTCTCCTCCTAAAGAGTAACGGAGGAGCACGAAGGTTGGCT | |
| | AATCCTGGTCGGACATCAGGAGGTTAGTGCAATGGCATAAGCCAGCTTGACTGCGAGCGTG | |
| | ACGGCGCGAGCAGGTGCGAAAGCAGGTCATAGTGATCCGGTGGTTCTGAATGGAAGGGCCA | |
| | TCGCTCAACGGATAAAAGGTACTCCGGGGATAACAGGCTGATACCGCCCAAGAGTTCATAT | |
| | CGACGGCGGTGTTTGGCACCTCGATGTCGGCTCATCACATCCTGGGGCTGAAGTAGGTCCC | |
| | AAGGGTATGGCTGTTCGCCATTTAAAGTGGTACGCGAGCTGGGTTTAGAACGTCGTGAGAC | |
| | AGTTCGGTCCCTATCTGCCGTGGGCGCTGGAGAACTGAGGGGGGCTGCTCCTAGTACGAGA | |
| | GGACCGGAGTGGACGCATCACTGGTGTTCGGGTTGTCATGCCAATGGCACTGCCCGGTAGC | |
| | TAAATGCGGAAGAGATAAGTGCTGAAAGCATCTAAGCACGAAACTTGCCCCGAGATGAGTT | |
| | CTCCCTGACCCTTTAAGGGTCCTGAAGGAACGTTGAAGACGACGACGTTGATAGGCCGGGT | |
| | GTGTAAGCGCAGCGATGCGTTGAGCTAACCGGTACTAATGAACCGTGAGGCTTAACCTTAC | |
| | AACGCCGAAGCTGTTTTGGCGGATGAGAGAAGATTTTCAGCCTGATACAGATTAAATCAGA | |
| | ACGCAGAAGCGGTCTGATAAAACAGAATTTGCCTGGCGGCAGTAGCGCGGTGGTCCCACCT | |
| | GACCCCATGCCGAACTCAGAAGTGAAACGCCGTAGCGCCGATGGTAGTGTGGGGTCTCCCC | |
| | ATGCGAGAGTAGGGAACTGCCAGGCATCAAATAAAACGAAAGGCTCAGTCGAAAGACTGGG | |
| | CCTTTCGTTTTATCTGTTGTTTGTCGGTGAACGCTCTCCTGAGTAGGACAAATCCGCCGGG | |
| | AGCGGATTTGAACGTTGCGAAGCAACGGCCCGGAGGGTGGCGGGCAGGACGCCCGCCATAA | |
| | ACTGCCAGGCATCAAATTAAGCAGAAGGCCATCCTGACGGATGGCCTTTTTGCGTTTCTAC | |
| | AAACTCTTCCTGTCGTCATATCTACAAGCCGGCGCGCCGGGAAATGTGCGCGGAACCCCTA | |
| | TTTGTTTATTTTTCTAAATACATTCAAATATGTATCCGCTCATGAGACAATAACCCTGATA | |
| | AATGCTTCAATAATATTGAAAAAGGAAGAGTATGAGTATTCAACATTTCCGTGTCGCCCTT | |
| | ATTCCCTTTTTTGCGGCATTTTGCCTTCCTGTTTTTGCTCACCCAGAAACGCTGGTGAAAG | |
| | TAAAAGATGCTGAAGATCAGTTGGGTGCACGAGTGGGTTACATCGAACTGGATCTCAACAG | |
| | CGGTAAGATCCTTGAGAGTTTTCGCCCCGAAGAACGTTTTCCAATGATGAGCACTTTTAAA | |
| | GTTCTGCTATGTGGCGCGGTATTATCCCGTGTTGACGCCGGGCAAGAGCAACTCGGTCGCC | |
| | GCATACACTATTCTCAGAATGACTTGGTTGAGTACTCACCAGTCACAGAAAAGCATCTTAC | |
| | GGATGGCATGACAGTAAGAGAATTATGCAGTGCTGCAATAACCATGAGTGATAACACTGCG | |
| | GCCAACTTACTTCTGACAACGATCGGAGGACCGAAGGAGCTAACCGCTTTTTTGCACAACA | |
| | TGGGGGATCATGTAACTCGCCTTGATCGTTGGGAACCGGAGCTGAATGAAGCCATACCAAA | |
| | CGACGAGCGTGACACCACGATGCCTGCAGCAATGGCAACAACGTTGCGCAAACTATTAACT | |
| | GGCGAACTACTTACTCTAGCTTCCCGGCAACAATTAATAGACTGGATGGAGGCGGATAAAG | |
| | TTGCAGGACCACTTCTGCGCTCGGCCCTTCCGGCTAGCTGGTTTATTGCTGATAAATCTGG | |
| | AGCCGGTGAGCGTGGGTCTCGCGGTATCATTGCAGCACTGGGGCCAGATGGTAAGCCCTCC | |
| | CGTATCGTAGTTATCTACACGACGGGGAGTCAGGCAACTATGGATGAACGAAATAGACAGA | |
| | TCGCTGAGATAGGTGCCTCACTGATTAAGCATTGGTAACTGCAGACCAAGTTTACTCATAT | |
| | ATACTTTAGATTGATTTAAAACTTCATTTTTAATTTAAAAGGATCTAGGTGAAGATCCTTT | |
| | TTGATAATCTCATGACCAAAATCCCTTAACGTGAGTTTTCGTTCCACTGAGCGTCAGACCC | |
| | CGTAGAAAAGATCAAAGGATCTTCTTGAGATCCTTTTTTTCTGCGCGTAATCTGCTGCTTG | |
| | CAAACAAAAAAACCACCGCTACCAGCGGTGGTTTGTTTGCCGGATCAAGAGCTACCAACTC | |
| | TTTTTCCGAAGGTAACTGGCTTCAGCAGAGCGCAGATACCAAATACTGTCCTTCTAGTGTA | |
| | GCCGTAGTTAGGCCACCACTTCAAGAACTCTGTAGCACCGCCTACATACCTCGCTCTGCTA | |
| | ATCCTGTTACCAGTGGCTGCTGCCAGTGGCGATAAGTCGTGTCTTACCGGGTTGGACTCAA | |
| | GACGATAGTTACCGGATAAGGCGCAGCGGTCGGGCTGAACGGGGGGTTCGTGCACACAGCC | |
| | CAGCTTGGAGCGAACGACCTACACCGAACTGAGATACCTACAGCGTGAGCTATGAGAAAGC | |
| | GCCACGCTTCCCGAAGGGAGAAAGGCGGACAGGTATCCGGTAAGCGGCAGGGTCGGAACAG | |
| | GAGAGCGCACGAGGGAGCTTCCAGGGGGAAACGCCTGGTATCTTTATAGTCCTGTCGGGTT | |
| | TCGCCACCTCTGACTTGAGCGTCGATTTTTGTGATGCTCGTCAGGGGGGCGGAGCCTATGG | |
| | AAAAACGCCAGCAACGCGGCCTTTTTACGGTTCCTGGCCTTTTGCTGG | |
| Δh26, 46 | GCGGCCGCGATCTCTCACCTACCAAACAATGCCCCCCTGCAAAAAATAAATTCATATAAAA | 110 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACTTGACGAGTGGCGGACGGGTGAGTAATGTCTGGGA | |
| | AACTGCCTGATGGAGGGGGATAACTACTGGAAACGGTAGCTAATACCGCATAACGTCGCAA | |
| | GACCAAAGAGGGGGACCTTCGGGCCTCTTGCCATCGGATGTGCCCAGATGGGATTAGCTAG | |
| | TAGGTGGGGTAACGGCTCACCTAGGCGACGATCCCTAGCTGGTCTGAGAGGATGACCAGCC | |
| | ACACTGGAACTGAGACACGGTCCAGACTCCTACGGGAGGCAGCAGTGGGGAATATTGCACA | |
| | ATGGGCGCAAGCCTGATGCAGCCATGCCGCGTGTATGAAGAAGGCCTTCGGGTTGTAAAGT | |
| | ACTTTCAGCGGGGAGGAAGGGAGTAAAGTTAATACCTTTGCTCATTGACGTTACCCGCAGA | |
| | AGAAGCACCGGCTAACTCCGTGCCAGCAGCCGCGGTAATACGGAGGGTGCAAGCGTTAATC | |
| | GGAATTACTGGGCGTAAAGCGCACGCAGGCGGTTTGTTAAGTCAGATGTGAAATCCCCGGG | |
| | CTCAACCTGGGAACTGCATCTGATACTGGCAAGCTTGAGTCTCGTAGAGGGGGGTAGAATT | |
| | CCAGGTGTAGCGGTGAAATGCGTAGAGATCTGGAGGAATACCGGTGGCGAAGGCGGCCCCC | |
| | TGGACGAAGACTGACGCTCAGGTGCGAAAGCGTGGGGAGCAAACAGGATTAGATACCCTGG | |
| | TAGTCCACGCCGTAAACGATGTCGACTTGGAGCTTGATTCCGGAGCTAACGCGTTAAGTCG | |
| | ACCGCCTGGGGAGTACGGCCGCAAGGTTAAAACTCAAATGAATTGACGGGGGCCCGCACAA | |
| | GCGGTGGAGCATGTGGTTTAATTCGATGCAACGCGAAGAACCTTACCTGGTCTTGACATCC | |
| | ACGGAAGTTTTCAGAGATGAGAATGTGCCTTCGGGAACCGTGAGACAGGTGCTGCATGGCT | |
| | GTCGTCAGCTCGTGTTGTGAAATGTTGGGTTAAGTCCCGCAACGAGCGCAACCCTTATCCT | |
| | TTGTTGCCAGCGGTCCGGCCGGGAACTCAAAGGAGACTGCCAGTGATAAACTGGAGGAAGG | |
| | TGGGGATGACGTCAAGTCATCATGGCCCTTACGACCAGGGCTACACACGTGCTACAATGGC | |
| | GCATACAAAGAGAAGCGACCTCGCGAGAGCAAGCGGACCTCATAAAGTGCGTCGTAGTCCG | |
| | GATTGGAGTCTGCAACTCGACTCCATGAAGTCGGAATCGCTAGTAATCGTGGATCAGAATG | |
| | CCACGGTGAATACGTTCCCGGGCCTTGTACACACCGCCCGTCACACCATGGGAGTGGGTTG | |
| | CAAAAGAAGTAGGTAGCTTAACCTTCGGGAGGGCGCTTACCACTTTGTGATTCATGACTGG | |
| | GGTGAAGTCGTAACAAGGTAACCGTAGGGGAACCTGCGGTTGGATCAtgtggtTACCTTAA | |
| | AGAAGCGTACTTTGTAGTGCTCACACAGATTGTCTGATAGAAAGTGAAAAGCAAGGCGTTT | |
| | ACGCGTTGGGAGTGAGGCTGAAGAGAATAAGGCCGTTCGCTTTCTATTAATGAAAGCTCAC | |
| | CCTACACGAAAATATCACGCAACGCGTGATAAGCAATTTTCGTGTCCCCTTCGTCTAGAGG | |
| | CCCAGGACACCGCCCTTTCACGGCGGTAACAGGGGTTCGAATCCCCTAGGGGACGCCACTT | |
| | GCTGGTTTGTGAGTGAAAGTCGCCGACCTTAATATCTCAAAACTCATCTTCGGGTGATGTT | |
| | TGAGATATTTGCTCTTTAAAAATCTGGATCAAGCTGAAAATTGAAACACTGAACAACGAGA | |
| | GTTGTTCGTGAGTCTCTCAAATTTTCGCAACACGATGATGAATCGAAAGAAACATCTTCGG | |
| | GTTGTGAGGTTAAGCGACTAAGCGTACACGGTGGATGCCCTGGCAGTCAGAGGCGATGAAG | |
| | GACGTGCTAATCTGCGATAAGCGTCGGTAAGGTGATATGAACCGTTATAACCGGCGATTTC | |
| | CGAATGGGGAAACCCAGTGTGTTTCGACACACTATCATTAACTGAATCCATAGGTTAATGA | |
| | GGCGAACCGGGGGAACTGAAACATCTAAGTACCCCGAGGAAAAGAAATCAACCGAGATTCC | |
| | CCCAGTAGCGGCGAGCGAACGGGGAGCAGCCCAGAGCCTGAATCAGTGTGTGTGTTAGTGG | |
| | AAGCGTCTGGAAAGGCGCGCGATACAGGGTGACAGCCCCGTACACAAAAATGCACATGCTG | |
| | TGAGCTCGATGAGTAGGGCGGGACACGTGGTATCCTGTCTGAATATGGGGGGACCATCCTC | |
| | CAAGGCTAAATACTCCTGACTGACCGATAGTGAACCAGTACCGTGAGGGAAAGGCGAAAAG | |
| | AACCCCGGCGAGGGGAGTGAAAAAGAACCTGAAACCGTGTACGTACAAGCAGTGGGAGCAC | |
| | GCTTAGGCGTGTGACTGCGTACCTTTTGTATAATGGGTCAGCGACTTATATTCTGTAGCAA | |
| | GGTTAACCGAATAGGGGAGCCGAAGGGAAACCGAGTCTTAACTGGGCGTTAAGTTGCAGGG | |
| | TATAGACCCGAAACCCGGTGATCTAGCCATGGGCAGGTTGAAGGTTGGGTAACACTAACTG | |
| | GAGGACCGAACCGACTAATGTTGAAAAATTAGCGGATGACTTGTGGCTGGGGGTGAAAGGC | |
| | CAATCAAACCGGGAGATAGCTGGTTCTCCCCGAAAGCTATTTAGGTAGCGCCTCGTGAATT | |
| | CATCTCCGGGGGTAGAGCACTGTTTCGGCAAGGGGGTCATCCCGACTTACCAACCCGATGC | |
| | AAACTGCGAATACCGGAGAATGTTATCACGGGAGACACACGGCGGGTGCTAACGTCCGTCG | |
| | TGAAGAGGGAAACAACCCAGACCGCCAGCTAAGGTCCCAAAGTCATGGTTAAGTGGGAAAC | |
| | GATGTGGGAAGGCCCAGACAGCCAGGATGTTGGCTTAGAAGCAGCCATCATTTAAAGAAAG | |
| | CGTAATAGCTCACTGGTCGAGTCGGCCTGCGCGGAAGATGTAACGGGGCTAAACCATGCAC | |
| | CGAAGCTGCGGCAGCGACGCTTATGCGTTGTTGGGTAGGGGAGCGTTCTGTAAGCCTGCGA | |
| | AGGTGTGCTGTGAGGCATGCTGGAGGTATCAGAAGTGCGAATGCTGACATAAGTAACGATA | |
| | AAGCGGGTGAAAAGCCCGCTCGCCGGAAGACCAAGGGTTCCTGTCCAACGTTAATCGGGGC | |
| | AGGGTGAGTCGACCCCTAAGGCGAGGCCGAAAGGCGTAGTCGATGGGAAACAGGTTAATAT | |
| | TCCTGTACTTGGTGTTACTGCGAAGGGGGGACGGAGAAGGCTATGTTGGCCGGGCGACGGT | |
| | TGTCCCGGTTTAAGCGTGTAGGCTGGTTTTCCAGGCAAATCCGGAAAATCAAGGCTGAGGC | |
| | GTGATGACGAGGCACTACGGTGCTGAAGCAACAAATGCCCTGCTTCCAGGAAAAGCCTCTA | |
| | AGCATCAGGTAACATCAAATCGTACCCCAAACCGACACAGGTGGTCAGGTAGAGAATACCA | |
| | AGGCGCTTGAGAGAACTCGGGTGAAGGAACTAGGCAAAATGGTGCCGTAACTTCGGGAGAA | |
| | GGCACGCTGATATGTAGGTGAGGTCCCTCGCGGATGGAGCTGAAATCAGTCGAAGATACCA | |
| | GCTGGCTGCAACTGTTTATTAAAAACACAGCACTGTGCAAACACGAAAGTGGACGTATACG | |
| | GTGTGACGCCTGCCCGGTGCCGGAAGGTTAATTGATGGGGTTAGCGCAAGCGAAGCTCTTG | |
| | ATCGAAGCCCCGGTAAACGGCGGCCGTAACTATAACGGTCCTAAGGTAGCGAAATTCCTTG | |
| | TCGGGTAAGTTCCGACCTGCACGAATGGCGTAATGATGGCCAGGCTGTCTCCACCCGAGAC | |
| | TCAGTGAAATTGAACTCGCTGTGAAGATGCAGTGTACCCGCGGCAAGACGGAAAGACCCCG | |
| | TGAACCTTTACTATAGCTTGACACTGAACATTGAGCCTTGATGTGTAGGATAGGTGGGAGG | |
| | CTTTGAAGTGTGGACGCCAGTCTGCATGGAGCCGACCTTGAAATACCACCCTTTAATGTTT | |
| | GATGTTCTAACGTTGACCCGTAATCCGGGTTGCGGACAGTGTCTGGTGGGTAGTTTGACTG | |
| | GGGCGGTCTCCTCCTAAAGAGTAACGGAGGAGCACGAAGGTTGGCTAATCCTGGTCGGACA | |
| | TCAGGAGGTTAGTGCAATGGCATAAGCCAGCTTGACTGCGAGCGTGACGGCGCGAGCAGGT | |
| | GCGAAAGCAGGTCATAGTGATCCGGTGGTTCTGAATGGAAGGGCCATCGCTCAACGGATAA | |
| | AAGGTACTCCGGGGATAACAGGCTGATACCGCCCAAGAGTTCATATCGACGGCGGTGTTTG | |
| | GCACCTCGATGTCGGCTCATCACATCCTGGGGCTGAAGTAGGTCCCAAGGGTATGGCTGTT | |
| | CGCCATTTAAAGTGGTACGCGAGCTGGGTTTAGAACGTCGTGAGACAGTTCGGTCCCTATC | |
| | TGCCGTGGGCGCTGGAGAACTGAGGGGGGCTGCTCCTAGTACGAGAGGACCGGAGTGGACG | |
| | CATCACTGGTGTTCGGGTTGTCATGCCAATGGCACTGCCCGGTAGCTAAATGCGGAAGAGA | |
| | TAAGTGCTGAAAGCATCTAAGCACGAAACTTGCCCCGAGATGAGTTCTCCCTGACCCTTTA | |
| | AGGGTCCTGAAGGAACGTTGAAGACGACGACGTTGATAGGCCGGGTGTGTAAGCGCAGCGA | |
| | TGCGTTGAGCTAACCGGTACTAATGAACCGTGAGGCTTAACCTTACAACGCCGAAGCTGTT | |
| | TTGGCGGATGAGAGAAGATTTTCAGCCTGATACAGATTAAATCAGAACGCAGAAGCGGTCT | |
| | GATAAAACAGAATTTGCCTGGCGGCAGTAGCGCGGTGGTCCCACCTGACCCCATGCCGAAC | |
| | TCAGAAGTGAAACGCCGTAGCGCCGATGGTAGTGTGGGGTCTCCCCATGCGAGAGTAGGGA | |
| | ACTGCCAGGCATCAAATAAAACGAAAGGCTCAGTCGAAAGACTGGGCCTTTCGTTTTATCT | |
| | GTTGTTTGTCGGTGAACGCTCTCCTGAGTAGGACAAATCCGCCGGGAGCGGATTTGAACGT | |
| | TGCGAAGCAACGGCCCGGAGGGTGGCGGGCAGGACGCCCGCCATAAACTGCCAGGCATCAA | |
| | ATTAAGCAGAAGGCCATCCTGACGGATGGCCTTTTTGCGTTTCTACAAACTCTTCCTGTCG | |
| | TCATATCTACAAGCCGGCGCGCCGGGAAATGTGCGCGGAACCCCTATTTGTTTATTTTTCT | |
| | AAATACATTCAAATATGTATCCGCTCATGAGACAATAACCCTGATAAATGCTTCAATAATA | |
| | TTGAAAAAGGAAGAGTATGAGTATTCAACATTTCCGTGTCGCCCTTATTCCCTTTTTTGCG | |
| | GCATTTTGCCTTCCTGTTTTTGCTCACCCAGAAACGCTGGTGAAAGTAAAAGATGCTGAAG | |
| | ATCAGTTGGGTGCACGAGTGGGTTACATCGAACTGGATCTCAACAGCGGTAAGATCCTTGA | |
| | GAGTTTTCGCCCCGAAGAACGTTTTCCAATGATGAGCACTTTTAAAGTTCTGCTATGTGGC | |
| | GCGGTATTATCCCGTGTTGACGCCGGGCAAGAGCAACTCGGTCGCCGCATACACTATTCTC | |
| | AGAATGACTTGGTTGAGTACTCACCAGTCACAGAAAAGCATCTTACGGATGGCATGACAGT | |
| | AAGAGAATTATGCAGTGCTGCAATAACCATGAGTGATAACACTGCGGCCAACTTACTTCTG | |
| | ACAACGATCGGAGGACCGAAGGAGCTAACCGCTTTTTTGCACAACATGGGGGATCATGTAA | |
| | CTCGCCTTGATCGTTGGGAACCGGAGCTGAATGAAGCCATACCAAACGACGAGCGTGACAC | |
| | CACGATGCCTGCAGCAATGGCAACAACGTTGCGCAAACTATTAACTGGCGAACTACTTACT | |
| | CTAGCTTCCCGGCAACAATTAATAGACTGGATGGAGGCGGATAAAGTTGCAGGACCACTTC | |
| | TGCGCTCGGCCCTTCCGGCTAGCTGGTTTATTGCTGATAAATCTGGAGCCGGTGAGCGTGG | |
| | GTCTCGCGGTATCATTGCAGCACTGGGGCCAGATGGTAAGCCCTCCCGTATCGTAGTTATC | |
| | TACACGACGGGGAGTCAGGCAACTATGGATGAACGAAATAGACAGATCGCTGAGATAGGTG | |
| | CCTCACTGATTAAGCATTGGTAACTGCAGACCAAGTTTACTCATATATACTTTAGATTGAT | |
| | TTAAAACTTCATTTTTAATTTAAAAGGATCTAGGTGAAGATCCTTTTTGATAATCTCATGA | |
| | CCAAAATCCCTTAACGTGAGTTTTCGTTCCACTGAGCGTCAGACCCCGTAGAAAAGATCAA | |
| | AGGATCTTCTTGAGATCCTTTTTTTCTGCGCGTAATCTGCTGCTTGCAAACAAAAAAACCA | |
| | CCGCTACCAGCGGTGGTTTGTTTGCCGGATCAAGAGCTACCAACTCTTTTTCCGAAGGTAA | |
| | CTGGCTTCAGCAGAGCGCAGATACCAAATACTGTCCTTCTAGTGTAGCCGTAGTTAGGCCA | |
| | CCACTTCAAGAACTCTGTAGCACCGCCTACATACCTCGCTCTGCTAATCCTGTTACCAGTG | |
| | GCTGCTGCCAGTGGCGATAAGTCGTGTCTTACCGGGTTGGACTCAAGACGATAGTTACCGG | |
| | ATAAGGCGCAGCGGTCGGGCTGAACGGGGGGTTCGTGCACACAGCCCAGCTTGGAGCGAAC | |
| | GACCTACACCGAACTGAGATACCTACAGCGTGAGCTATGAGAAAGCGCCACGCTTCCCGAA | |
| | GGGAGAAAGGCGGACAGGTATCCGGTAAGCGGCAGGGTCGGAACAGGAGAGCGCACGAGGG | |
| | AGCTTCCAGGGGGAAACGCCTGGTATCTTTATAGTCCTGTCGGGTTTCGCCACCTCTGACT | |
| | TGAGCGTCGATTTTTGTGATGCTCGTCAGGGGGGCGGAGCCTATGGAAAAACGCCAGCAAC | |
| | GCGGCCTTTTTACGGTTCCTGGCCTTTTGCTGG | |
| Δh26, 48 | GCGGCCGCGATCTCTCACCTACCAAACAATGCCCCCCTGCAAAAAATAAATTCATATAAAA | 111 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACGCTAATACCGCATAAC | |
| | GTCGCAAGACCAAAGAGGGGGACCTTCGGGCCTCTTGCCATCGGATGTGCCCAGATGGGAT | |
| | TAGCTAGTAGGTGGGGTAACGGCTCACCTAGGCGACGATCCCTAGCTGGTCTGAGAGGATG | |
| | ACCAGCCACACTGGAACTGAGACACGGTCCAGACTCCTACGGGAGGCAGCAGTGGGGAATA | |
| | TTGCACAATGGGCGCAAGCCTGATGCAGCCATGCCGCGTGTATGAAGAAGGCCTTCGGGTT | |
| | GTAAAGTACTTTCAGCGGGGAGGAAGGGAGTAAAGTTAATACCTTTGCTCATTGACGTTAC | |
| | CCGCAGAAGAAGCACCGGCTAACTCCGTGCCAGCAGCCGCGGTAATACGGAGGGTGCAAGC | |
| | GTTAATCGGAATTACTGGGCGTAAAGCGCACGCAGGCGGTTTGTTAAGTCAGATGTGAAAT | |
| | CCCCGGGCTCAACCTGGGAACTGCATCTGATACTGGCAAGCTTGAGTCTCGTAGAGGGGGG | |
| | TAGAATTCCAGGTGTAGCGGTGAAATGCGTAGAGATCTGGAGGAATACCGGTGGCGAAGGC | |
| | GGCCCCCTGGACGAAGACTGACGCTCAGGTGCGAAAGCGTGGGGAGCAAACAGGATTAGAT | |
| | ACCCTGGTAGTCCACGCCGTAAACGATGTCGACTTGGAGCTTGATTCCGGAGCTAACGCGT | |
| | TAAGTCGACCGCCTGGGGAGTACGGCCGCAAGGTTAAAACTCAAATGAATTGACGGGGGCC | |
| | CGCACAAGCGGTGGAGCATGTGGTTTAATTCGATGCAACGCGAAGAACCTTACCTGGTCTT | |
| | GACATCCACGGAAGTTTTCAGAGATGAGAATGTGCCTTCGGGAACCGTGAGACAGGTGCTG | |
| | CATGGCTGTCGTCAGCTCGTGTTGTGAAATGTTGGGTTAAGTCCCGCAACGAGCGCAACCC | |
| | TTATCCTTTGTTGCCAGCGGTCCGGCCGGGAACTCAAAGGAGACTGCCAGTGATAAACTGG | |
| | AGGAAGGTGGGGATGACGTCAAGTCATCATGGCCCTTACGACCAGGGCTACACACGTGCTA | |
| | CAATGGCGCATACAAAGAGAAGCGACCTCGCGAGAGCAAGCGGACCTCATAAAGTGCGTCG | |
| | TAGTCCGGATTGGAGTCTGCAACTCGACTCCATGAAGTCGGAATCGCTAGTAATCGTGGAT | |
| | CAGAATGCCACGGTGAATACGTTCCCGGGCCTTGTACACACCGCCCGTCACACCATGGGAG | |
| | TGGGTTGCAAAAGAAGTAGGTAGCTTAACCTTCGGGAGGGCGCTTACCACTTTGTGATTCA | |
| | TGACTGGGGTGAAGTCGTAACAAGGTAACCGTAGGGGAACCTGCGGTTGGATCAtgtggtT | |
| | ACCTTAAAGAAGCGTACTTTGTAGTGCTCACACAGATTGTCTGATAGAAAGTGAAAAGCAA | |
| | GGCGTTTACGCGTTGGGAGTGAGGCTGAAGAGAATAAGGCCGTTCGCTTTCTATTAATGAA | |
| | AGCTCACCCTACACGAAAATATCACGCAACGCGTGATAAGCAATTTTCGTGTCCCCTTCGT | |
| | CTAGAGGCCCAGGACACCGCCCTTTCACGGCGGTAACAGGGGTTCGAATCCCCTAGGGGAC | |
| | GCCACTTGCTGGTTTGTGAGTGAAAGTCGCCGACCTTAATATCTCAAAACTCATCTTCGGG | |
| | TGATGTTTGAGATATTTGCTCTTTAAAAATCTGGATCAAGCTGAAAATTGAAACACTGAAC | |
| | AACGAGAGTTGTTCGTGAGTCTCTCAAATTTTCGCAACACGATGATGAATCGAAAGAAACA | |
| | TCTTCGGGTTGTGAGGTTAAGCGACTAAGCGTACACGGTGGATGCCCTGGCAGTCAGAGGC | |
| | GATGAAGGACGTGCTAATCTGCGATAAGCGTCGGTAAGGTGATATGAACCGTTATAACCGG | |
| | CGATTTCCGAATGGGGAAACCCAGTGTGTTTCGACACACTATCATTAACTGAATCCATAGG | |
| | TTAATGAGGCGAACCGGGGGAACTGAAACATCTAAGTACCCCGAGGAAAAGAAATCAACCG | |
| | AGATTCCCCCAGTAGCGGCGAGCGAACGGGGAGCAGCCCAGAGCCTGAATCAGTGTGTGTG | |
| | TTAGTGGAAGCGTCTGGAAAGGCGCGCGATACAGGGTGACAGCCCCGTACACAAAAATGCA | |
| | CATGCTGTGAGCTCGATGAGTAGGGCGGGACACGTGGTATCCTGTCTGAATATGGGGGGAC | |
| | CATCCTCCAAGGCTAAATACTCCTGACTGACCGATAGTGAACCAGTACCGTGAGGGAAAGG | |
| | CGAAAAGAACCCCGGCGAGGGGAGTGAAAAAGAACCTGAAACCGTGTACGTACAAGCAGTG | |
| | GGAGCACGCTTAGGCGTGTGACTGCGTACCTTTTGTATAATGGGTCAGCGACTTATATTCT | |
| | GTAGCAAGGTTAACCGAATAGGGGAGCCGAAGGGAAACCGAGTCTTAACTGGGCGTTAAGT | |
| | TGCAGGGTATAGACCCGAAACCCGGTGATCTAGCCATGGGCAGGTTGAAGGTTGGGTAACA | |
| | CTAACTGGAGGACCGAACCGACTAATGTTGAAAAATTAGCGGATGACTTGTGGCTGGGGGT | |
| | GAAAGGCCAATCAAACCGGGAGATAGCTGGTTCTCCCCGAAAGCTATTTAGGTAGCGCCTC | |
| | GTGAATTCATCTCCGGGGGTAGAGCACTGTTTCGGCAAGGGGGTCATCCCGACTTACCAAC | |
| | CCGATGCAAACTGCGAATACCGGAGAATGTTATCACGGGAGACACACGGCGGGTGCTAACG | |
| | TCCGTCGTGAAGAGGGAAACAACCCAGACCGCCAGCTAAGGTCCCAAAGTCATGGTTAAGT | |
| | GGGAAACGATGTGGGAAGGCCCAGACAGCCAGGATGTTGGCTTAGAAGCAGCCATCATTTA | |
| | AAGAAAGCGTAATAGCTCACTGGTCGAGTCGGCCTGCGCGGAAGATGTAACGGGGCTAAAC | |
| | CATGCACCGAAGCTGCGGCAGCGACGCTTATGCGTTGTTGGGTAGGGGAGCGTTCTGTAAG | |
| | CCTGCGAAGGTGTGCTGTGAGGCATGCTGGAGGTATCAGAAGTGCGAATGCTGACATAAGT | |
| | AACGATAAAGCGGGTGAAAAGCCCGCTCGCCGGAAGACCAAGGGTTCCTGTCCAACGTTAA | |
| | TCGGGGCAGGGTGAGTCGACCCCTAAGGCGAGGCCGAAAGGCGTAGTCGATGGGAAACAGG | |
| | TTAATATTCCTGTACTTGGTGTTACTGCGAAGGGGGGACGGAGAAGGCTATGTTGGCCGGG | |
| | CGACGGTTGTCCCGGTTTAAGCGTGTAGGCTGGTTTTCCAGGCAAATCCGGAAAATCAAGG | |
| | CTGAGGCGTGATGACGAGGCACTACGGTGCTGAAGCAACAAATGCCCTGCTTCCAGGAAAA | |
| | GCCTCTAAGCATCAGGTAACATCAAATCGTACCCCAAACCGACACAGGTGGTCAGGTAGAG | |
| | AATACCAAGGCGCTTGAGAGAACTCGGGTGAAGGAACTAGGCAAAATGGTGCCGTAACTTC | |
| | GGGAGAAGGCACGCTGATATGTAGGTGAGGTCCCTCGCGGATGGAGCTGAAATCAGTCGAA | |
| | GATACCAGCTGGCTGCAACTGTTTATTAAAAACACAGCACTGTGCAAACACGAAAGTGGAC | |
| | GTATACGGTGTGACGCCTGCCCGGTGCCGGAAGGTTAATTGATGGGGTTAGCGCAAGCGAA | |
| | GCTCTTGATCGAAGCCCCGGTAAACGGCGGCCGTAACTATAACGGTCCTAAGGTAGCGAAA | |
| | TTCCTTGTCGGGTAAGTTCCGACCTGCACGAATGGCGTAATGATGGCCAGGCTGTCTCCAC | |
| | CCGAGACTCAGTGAAATTGAACTCGCTGTGAAGATGCAGTGTACCCGCGGCAAGACGGAAA | |
| | GACCCCGTGAACCTTTACTATAGCTTGACACTGAACATTGAGCCTTGATGTGTAGGATAGG | |
| | TGGGAGGCTTTGAAGTGTGGACGCCAGTCTGCATGGAGCCGACCTTGAAATACCACCCTTT | |
| | AATGTTTGATGTTCTAACGTTGACCCGTAATCCGGGTTGCGGACAGTGTCTGGTGGGTAGT | |
| | TTGACTGGGGCGGTCTCCTCCTAAAGAGTAACGGAGGAGCACGAAGGTTGGCTAATCCTGG | |
| | TCGGACATCAGGAGGTTAGTGCAATGGCATAAGCCAGCTTGACTGCGAGCGTGACGGCGCG | |
| | AGCAGGTGCGAAAGCAGGTCATAGTGATCCGGTGGTTCTGAATGGAAGGGCCATCGCTCAA | |
| | CGGATAAAAGGTACTCCGGGGATAACAGGCTGATACCGCCCAAGAGTTCATATCGACGGCG | |
| | GTGTTTGGCACCTCGATGTCGGCTCATCACATCCTGGGGCTGAAGTAGGTCCCAAGGGTAT | |
| | GGCTGTTCGCCATTTAAAGTGGTACGCGAGCTGGGTTTAGAACGTCGTGAGACAGTTCGGT | |
| | CCCTATCTGCCGTGGGCGCTGGAGAACTGAGGGGGGCTGCTCCTAGTACGAGAGGACCGGA | |
| | GTGGACGCATCACTGGTGTTCGGGTTGTCATGCCAATGGCACTGCCCGGTAGCTAAATGCG | |
| | GAAGAGATAAGTGCTGAAAGCATCTAAGCACGAAACTTGCCCCGAGATGAGTTCTCCCTGA | |
| | CCCTTTAAGGGTCCTGAAGGAACGTTGAAGACGACGACGTTGATAGGCCGGGTGTGTAAGC | |
| | GCAGCGATGCGTTGAGCTAACCGGTACTAATGAACCGTGAGGCTTAACCTTACAACGCCGA | |
| | AGCTGTTTTGGCGGATGAGAGAAGATTTTCAGCCTGATACAGATTAAATCAGAACGCAGAA | |
| | GCGGTCTGATAAAACAGAATTTGCCTGGCGGCAGTAGCGCGGTGGTCCCACCTGACCCCAT | |
| | GCCGAACTCAGAAGTGAAACGCCGTAGCGCCGATGGTAGTGTGGGGTCTCCCCATGCGAGA | |
| | GTAGGGAACTGCCAGGCATCAAATAAAACGAAAGGCTCAGTCGAAAGACTGGGCCTTTCGT | |
| | TTTATCTGTTGTTTGTCGGTGAACGCTCTCCTGAGTAGGACAAATCCGCCGGGAGCGGATT | |
| | TGAACGTTGCGAAGCAACGGCCCGGAGGGTGGCGGGCAGGACGCCCGCCATAAACTGCCAG | |
| | GCATCAAATTAAGCAGAAGGCCATCCTGACGGATGGCCTTTTTGCGTTTCTACAAACTCTT | |
| | CCTGTCGTCATATCTACAAGCCGGCGCGCCGGGAAATGTGCGCGGAACCCCTATTTGTTTA | |
| | TTTTTCTAAATACATTCAAATATGTATCCGCTCATGAGACAATAACCCTGATAAATGCTTC | |
| | AATAATATTGAAAAAGGAAGAGTATGAGTATTCAACATTTCCGTGTCGCCCTTATTCCCTT | |
| | TTTTGCGGCATTTTGCCTTCCTGTTTTTGCTCACCCAGAAACGCTGGTGAAAGTAAAAGAT | |
| | GCTGAAGATCAGTTGGGTGCACGAGTGGGTTACATCGAACTGGATCTCAACAGCGGTAAGA | |
| | TCCTTGAGAGTTTTCGCCCCGAAGAACGTTTTCCAATGATGAGCACTTTTAAAGTTCTGCT | |
| | ATGTGGCGCGGTATTATCCCGTGTTGACGCCGGGCAAGAGCAACTCGGTCGCCGCATACAC | |
| | TATTCTCAGAATGACTTGGTTGAGTACTCACCAGTCACAGAAAAGCATCTTACGGATGGCA | |
| | TGACAGTAAGAGAATTATGCAGTGCTGCAATAACCATGAGTGATAACACTGCGGCCAACTT | |
| | ACTTCTGACAACGATCGGAGGACCGAAGGAGCTAACCGCTTTTTTGCACAACATGGGGGAT | |
| | CATGTAACTCGCCTTGATCGTTGGGAACCGGAGCTGAATGAAGCCATACCAAACGACGAGC | |
| | GTGACACCACGATGCCTGCAGCAATGGCAACAACGTTGCGCAAACTATTAACTGGCGAACT | |
| | ACTTACTCTAGCTTCCCGGCAACAATTAATAGACTGGATGGAGGCGGATAAAGTTGCAGGA | |
| | CCACTTCTGCGCTCGGCCCTTCCGGCTAGCTGGTTTATTGCTGATAAATCTGGAGCCGGTG | |
| | AGCGTGGGTCTCGCGGTATCATTGCAGCACTGGGGCCAGATGGTAAGCCCTCCCGTATCGT | |
| | AGTTATCTACACGACGGGGAGTCAGGCAACTATGGATGAACGAAATAGACAGATCGCTGAG | |
| | ATAGGTGCCTCACTGATTAAGCATTGGTAACTGCAGACCAAGTTTACTCATATATACTTTA | |
| | GATTGATTTAAAACTTCATTTTTAATTTAAAAGGATCTAGGTGAAGATCCTTTTTGATAAT | |
| | CTCATGACCAAAATCCCTTAACGTGAGTTTTCGTTCCACTGAGCGTCAGACCCCGTAGAAA | |
| | AGATCAAAGGATCTTCTTGAGATCCTTTTTTTCTGCGCGTAATCTGCTGCTTGCAAACAAA | |
| | AAAACCACCGCTACCAGCGGTGGTTTGTTTGCCGGATCAAGAGCTACCAACTCTTTTTCCG | |
| | AAGGTAACTGGCTTCAGCAGAGCGCAGATACCAAATACTGTCCTTCTAGTGTAGCCGTAGT | |
| | TAGGCCACCACTTCAAGAACTCTGTAGCACCGCCTACATACCTCGCTCTGCTAATCCTGTT | |
| | ACCAGTGGCTGCTGCCAGTGGCGATAAGTCGTGTCTTACCGGGTTGGACTCAAGACGATAG | |
| | TTACCGGATAAGGCGCAGCGGTCGGGCTGAACGGGGGGTTCGTGCACACAGCCCAGCTTGG | |
| | AGCGAACGACCTACACCGAACTGAGATACCTACAGCGTGAGCTATGAGAAAGCGCCACGCT | |
| | TCCCGAAGGGAGAAAGGCGGACAGGTATCCGGTAAGCGGCAGGGTCGGAACAGGAGAGCGC | |
| | ACGAGGGAGCTTCCAGGGGGAAACGCCTGGTATCTTTATAGTCCTGTCGGGTTTCGCCACC | |
| | TCTGACTTGAGCGTCGATTTTTGTGATGCTCGTCAGGGGGGCGGAGCCTATGGAAAAACGC | |
| | CAGCAACGCGGCCTTTTTACGGTTCCTGGCCTTTTGCTGG | |
| Δh26, 433 | GCGGCCGCGATCTCTCACCTACCAAACAATGCCCCCCTGCAAAAAATAAATTCATATAAAA | 112 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACTACTGGAAACGGTAGC | |
| | TAATACCGCATAACGTCGCAAGACCAAAGAGGGGGACCTTCGGGCCTCTTGCCATCGGATG | |
| | TGCCCAGATGGGATTAGCTAGTAGGTGGGGTAACGGCTCACCTAGGCGACGATCCCTAGCT | |
| | GGTCTGAGAGGATGACCAGCCACACTGGAACTGAGACACGGTCCAGACTCCTACGGGAGGC | |
| | AGCAGTGGGGAATATTGCACAATGGGCGCAAGCCTGATGCAGCCATGCCGCGTGTATGAAG | |
| | AAGGCCTTCGGGTTGTAAAGTACTTTCAGCGGGGAGGAAGGGAGTAAAGTTAATACCTTTG | |
| | CTCATTGACGTTACCCGCAGAAGAAGCACCGGCTAACTCCGTGCCAGCAGCCGCGGTAATA | |
| | CGGAGGGTGCAAGCGTTAATCGGAATTACTGGGCGTAAAGCGCACGCAGGCGGTTTGTTAA | |
| | GTCAGATGTGAAATCCCCGGGCTCAACCTGGGAACTGCATCTGATACTGGCAAGCTTGAGT | |
| | CTCGTAGAGGGGGGTAGAATTCCAGGTGTAGCGGTGAAATGCGTAGAGATCTGGAGGAATA | |
| | CCGGTGGCGAAGGCGGCCCCCTGGACGAAGACTGACGCTCAGGTGCGAAAGCGTGGGGAGC | |
| | AAACAGGATTAGATACCCTGGTAGTCCACGCCGTAAACGATGTCGACTTGGAGCTTGATTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTGGTCTTGACATCCGAGACAGGTGCTGCATGGCTGTCGTCAGCTCGTGTTGTGA | |
| | AATGTTGGGTTAAGTCCCGCAACGAGCGCAACCCTTATCCTTTGTTGCCAGCGGTCCGGCC | |
| | GGGAACTCAAAGGAGACTGCCAGTGATAAACTGGAGGAAGGTGGGGATGACGTCAAGTCAT | |
| | CATGGCCCTTACGACCAGGGCTACACACGTGCTACAATGGCGCATACAAAGAGAAGCGACC | |
| | TCGCGAGAGCAAGCGGACCTCATAAAGTGCGTCGTAGTCCGGATTGGAGTCTGCAACTCGA | |
| | CTCCATGAAGTCGGAATCGCTAGTAATCGTGGATCAGAATGCCACGGTGAATACGTTCCCG | |
| | GGCCTTGTACACACCGCCCGTCACACCATGGGAGTGGGTTGCAAAAGAAGTAGGTAGCTTA | |
| | ACCTTCGGGAGGGCGCTTACCACTTTGTGATTCATGACTGGGGTGAAGTCGTAACAAGGTA | |
| | ACCGTAGGGGAACCTGCGGTTGGATCAtgtggtTACCTTAAAGAAGCGTACTTTGTAGTGC | |
| | TCACACAGATTGTCTGATAGAAAGTGAAAAGCAAGGCGTTTACGCGTTGGGAGTGAGGCTG | |
| | AAGAGAATAAGGCCGTTCGCTTTCTATTAATGAAAGCTCACCCTACACGAAAATATCACGC | |
| | AACGCGTGATAAGCAATTTTCGTGTCCCCTTCGTCTAGAGGCCCAGGACACCGCCCTTTCA | |
| | CGGCGGTAACAGGGGTTCGAATCCCCTAGGGGACGCCACTTGCTGGTTTGTGAGTGAAAGT | |
| | CGCCGACCTTAATATCTCAAAACTCATCTTCGGGTGATGTTTGAGATATTTGCTCTTTAAA | |
| | AATCTGGATCAAGCTGAAAATTGAAACACTGAACAACGAGAGTTGTTCGTGAGTCTCTCAA | |
| | ATTTTCGCAACACGATGATGAATCGAAAGAAACATCTTCGGGTTGTGAGGTTAAGCGACTA | |
| | AGCGTACACGGTGGATGCCCTGGCAGTCAGAGGCGATGAAGGACGTGCTAATCTGCGATAA | |
| | GCGTCGGTAAGGTGATATGAACCGTTATAACCGGCGATTTCCGAATGGGGAAACCCAGTGT | |
| | GTTTCGACACACTATCATTAACTGAATCCATAGGTTAATGAGGCGAACCGGGGGAACTGAA | |
| | ACATCTAAGTACCCCGAGGAAAAGAAATCAACCGAGATTCCCCCAGTAGCGGCGAGCGAAC | |
| | GGGGAGCAGCCCAGAGCCTGAATCAGTGTGTGTGTTAGTGGAAGCGTCTGGAAAGGCGCGC | |
| | GATACAGGGTGACAGCCCCGTACACAAAAATGCACATGCTGTGAGCTCGATGAGTAGGGCG | |
| | GGACACGTGGTATCCTGTCTGAATATGGGGGGACCATCCTCCAAGGCTAAATACTCCTGAC | |
| | TGACCGATAGTGAACCAGTACCGTGAGGGAAAGGCGAAAAGAACCCCGGCGAGGGGAGTGA | |
| | AAAAGAACCTGAAACCGTGTACGTACAAGCAGTGGGAGCACGCTTAGGCGTGTGACTGCGT | |
| | ACCTTTTGTATAATGGGTCAGCGACTTATATTCTGTAGCAAGGTTAACCGAATAGGGGAGC | |
| | CGAAGGGAAACCGAGTCTTAACTGGGCGTTAAGTTGCAGGGTATAGACCCGAAACCCGGTG | |
| | ATCTAGCCATGGGCAGGTTGAAGGTTGGGTAACACTAACTGGAGGACCGAACCGACTAATG | |
| | TTGAAAAATTAGCGGATGACTTGTGGCTGGGGGTGAAAGGCCAATCAAACCGGGAGATAGC | |
| | TGGTTCTCCCCGAAAGCTATTTAGGTAGCGCCTCGTGAATTCATCTCCGGGGGTAGAGCAC | |
| | TGTTTCGGCAAGGGGGTCATCCCGACTTACCAACCCGATGCAAACTGCGAATACCGGAGAA | |
| | TGTTATCACGGGAGACACACGGCGGGTGCTAACGTCCGTCGTGAAGAGGGAAACAACCCAG | |
| | ACCGCCAGCTAAGGTCCCAAAGTCATGGTTAAGTGGGAAACGATGTGGGAAGGCCCAGACA | |
| | GCCAGGATGTTGGCTTAGAAGCAGCCATCATTTAAAGAAAGCGTAATAGCTCACTGGTCGA | |
| | GTCGGCCTGCGCGGAAGATGTAACGGGGCTAAACCATGCACCGAAGCTGCGGCAGCGACGC | |
| | TTATGCGTTGTTGGGTAGGGGAGCGTTCTGTAAGCCTGCGAAGGTGTGCTGTGAGGCATGC | |
| | TGGAGGTATCAGAAGTGCGAATGCTGACATAAGTAACGATAAAGCGGGTGAAAAGCCCGCT | |
| | CGCCGGAAGACCAAGGGTTCCTGTCCAACGTTAATCGGGGCAGGGTGAGTCGACCCCTAAG | |
| | GCGAGGCCGAAAGGCGTAGTCGATGGGAAACAGGTTAATATTCCTGTACTTGGTGTTACTG | |
| | CGAAGGGGGGACGGAGAAGGCTATGTTGGCCGGGCGACGGTTGTCCCGGTTTAAGCGTGTA | |
| | GGCTGGTTTTCCAGGCAAATCCGGAAAATCAAGGCTGAGGCGTGATGACGAGGCACTACGG | |
| | TGCTGAAGCAACAAATGCCCTGCTTCCAGGAAAAGCCTCTAAGCATCAGGTAACATCAAAT | |
| | CGTACCCCAAACCGACACAGGTGGTCAGGTAGAGAATACCAAGGCGCTTGAGAGAACTCGG | |
| | GTGAAGGAACTAGGCAAAATGGTGCCGTAACTTCGGGAGAAGGCACGCTGATATGTAGGTG | |
| | AGGTCCCTCGCGGATGGAGCTGAAATCAGTCGAAGATACCAGCTGGCTGCAACTGTTTATT | |
| | AAAAACACAGCACTGTGCAAACACGAAAGTGGACGTATACGGTGTGACGCCTGCCCGGTGC | |
| | CGGAAGGTTAATTGATGGGGTTAGCGCAAGCGAAGCTCTTGATCGAAGCCCCGGTAAACGG | |
| | CGGCCGTAACTATAACGGTCCTAAGGTAGCGAAATTCCTTGTCGGGTAAGTTCCGACCTGC | |
| | ACGAATGGCGTAATGATGGCCAGGCTGTCTCCACCCGAGACTCAGTGAAATTGAACTCGCT | |
| | GTGAAGATGCAGTGTACCCGCGGCAAGACGGAAAGACCCCGTGAACCTTTACTATAGCTTG | |
| | ACACTGAACATTGAGCCTTGATGTGTAGGATAGGTGGGAGGCTTTGAAGTGTGGACGCCAG | |
| | TCTGCATGGAGCCGACCTTGAAATACCACCCTTTAATGTTTGATGTTCTAACGTTGACCCG | |
| | TAATCCGGGTTGCGGACAGTGTCTGGTGGGTAGTTTGACTGGGGCGGTCTCCTCCTAAAGA | |
| | GTAACGGAGGAGCACGAAGGTTGGCTAATCCTGGTCGGACATCAGGAGGTTAGTGCAATGG | |
| | CATAAGCCAGCTTGACTGCGAGCGTGACGGCGCGAGCAGGTGCGAAAGCAGGTCATAGTGA | |
| | TCCGGTGGTTCTGAATGGAAGGGCCATCGCTCAACGGATAAAAGGTACTCCGGGGATAACA | |
| | GGCTGATACCGCCCAAGAGTTCATATCGACGGCGGTGTTTGGCACCTCGATGTCGGCTCAT | |
| | CACATCCTGGGGCTGAAGTAGGTCCCAAGGGTATGGCTGTTCGCCATTTAAAGTGGTACGC | |
| | GAGCTGGGTTTAGAACGTCGTGAGACAGTTCGGTCCCTATCTGCCGTGGGCGCTGGAGAAC | |
| | TGAGGGGGGCTGCTCCTAGTACGAGAGGACCGGAGTGGACGCATCACTGGTGTTCGGGTTG | |
| | TCATGCCAATGGCACTGCCCGGTAGCTAAATGCGGAAGAGATAAGTGCTGAAAGCATCTAA | |
| | GCACGAAACTTGCCCCGAGATGAGTTCTCCCTGACCCTTTAAGGGTCCTGAAGGAACGTTG | |
| | AAGACGACGACGTTGATAGGCCGGGTGTGTAAGCGCAGCGATGCGTTGAGCTAACCGGTAC | |
| | TAATGAACCGTGAGGCTTAACCTTACAACGCCGAAGCTGTTTTGGCGGATGAGAGAAGATT | |
| | TTCAGCCTGATACAGATTAAATCAGAACGCAGAAGCGGTCTGATAAAACAGAATTTGCCTG | |
| | GCGGCAGTAGCGCGGTGGTCCCACCTGACCCCATGCCGAACTCAGAAGTGAAACGCCGTAG | |
| | CGCCGATGGTAGTGTGGGGTCTCCCCATGCGAGAGTAGGGAACTGCCAGGCATCAAATAAA | |
| | ACGAAAGGCTCAGTCGAAAGACTGGGCCTTTCGTTTTATCTGTTGTTTGTCGGTGAACGCT | |
| | CTCCTGAGTAGGACAAATCCGCCGGGAGCGGATTTGAACGTTGCGAAGCAACGGCCCGGAG | |
| | GGTGGCGGGCAGGACGCCCGCCATAAACTGCCAGGCATCAAATTAAGCAGAAGGCCATCCT | |
| | GACGGATGGCCTTTTTGCGTTTCTACAAACTCTTCCTGTCGTCATATCTACAAGCCGGCGC | |
| | GCCGGGAAATGTGCGCGGAACCCCTATTTGTTTATTTTTCTAAATACATTCAAATATGTAT | |
| | CCGCTCATGAGACAATAACCCTGATAAATGCTTCAATAATATTGAAAAAGGAAGAGTATGA | |
| | GTATTCAACATTTCCGTGTCGCCCTTATTCCCTTTTTTGCGGCATTTTGCCTTCCTGTTTT | |
| | TGCTCACCCAGAAACGCTGGTGAAAGTAAAAGATGCTGAAGATCAGTTGGGTGCACGAGTG | |
| | GGTTACATCGAACTGGATCTCAACAGCGGTAAGATCCTTGAGAGTTTTCGCCCCGAAGAAC | |
| | GTTTTCCAATGATGAGCACTTTTAAAGTTCTGCTATGTGGCGCGGTATTATCCCGTGTTGA | |
| | CGCCGGGCAAGAGCAACTCGGTCGCCGCATACACTATTCTCAGAATGACTTGGTTGAGTAC | |
| | TCACCAGTCACAGAAAAGCATCTTACGGATGGCATGACAGTAAGAGAATTATGCAGTGCTG | |
| | CAATAACCATGAGTGATAACACTGCGGCCAACTTACTTCTGACAACGATCGGAGGACCGAA | |
| | GGAGCTAACCGCTTTTTTGCACAACATGGGGGATCATGTAACTCGCCTTGATCGTTGGGAA | |
| | CCGGAGCTGAATGAAGCCATACCAAACGACGAGCGTGACACCACGATGCCTGCAGCAATGG | |
| | CAACAACGTTGCGCAAACTATTAACTGGCGAACTACTTACTCTAGCTTCCCGGCAACAATT | |
| | AATAGACTGGATGGAGGCGGATAAAGTTGCAGGACCACTTCTGCGCTCGGCCCTTCCGGCT | |
| | AGCTGGTTTATTGCTGATAAATCTGGAGCCGGTGAGCGTGGGTCTCGCGGTATCATTGCAG | |
| | CACTGGGGCCAGATGGTAAGCCCTCCCGTATCGTAGTTATCTACACGACGGGGAGTCAGGC | |
| | AACTATGGATGAACGAAATAGACAGATCGCTGAGATAGGTGCCTCACTGATTAAGCATTGG | |
| | TAACTGCAGACCAAGTTTACTCATATATACTTTAGATTGATTTAAAACTTCATTTTTAATT | |
| | TAAAAGGATCTAGGTGAAGATCCTTTTTGATAATCTCATGACCAAAATCCCTTAACGTGAG | |
| | TTTTCGTTCCACTGAGCGTCAGACCCCGTAGAAAAGATCAAAGGATCTTCTTGAGATCCTT | |
| | TTTTTCTGCGCGTAATCTGCTGCTTGCAAACAAAAAAACCACCGCTACCAGCGGTGGTTTG | |
| | TTTGCCGGATCAAGAGCTACCAACTCTTTTTCCGAAGGTAACTGGCTTCAGCAGAGCGCAG | |
| | ATACCAAATACTGTCCTTCTAGTGTAGCCGTAGTTAGGCCACCACTTCAAGAACTCTGTAG | |
| | CACCGCCTACATACCTCGCTCTGCTAATCCTGTTACCAGTGGCTGCTGCCAGTGGCGATAA | |
| | GTCGTGTCTTACCGGGTTGGACTCAAGACGATAGTTACCGGATAAGGCGCAGCGGTCGGGC | |
| | TGAACGGGGGGTTCGTGCACACAGCCCAGCTTGGAGCGAACGACCTACACCGAACTGAGAT | |
| | ACCTACAGCGTGAGCTATGAGAAAGCGCCACGCTTCCCGAAGGGAGAAAGGCGGACAGGTA | |
| | TCCGGTAAGCGGCAGGGTCGGAACAGGAGAGCGCACGAGGGAGCTTCCAGGGGGAAACGCC | |
| | TGGTATCTTTATAGTCCTGTCGGGTTTCGCCACCTCTGACTTGAGCGTCGATTTTTGTGAT | |
| | GCTCGTCAGGGGGGCGGAGCCTATGGAAAAACGCCAGCAACGCGGCCTTTTTACGGTTCCT | |
| | GGCCTTTTGCTGG | |
| Δh26, Δ41 | GCGGCCGCGATCTCTCACCTACCAAACAATGCCCCCCTGCAAAAAATAAATTCATATAAAA | 113 |
| | AACATACAGATAACCATCTGCGGTGAtccctatcagtgatagagaTTGACAtccctatcag | |
| | tgatagagATACTGAGCACGGGTACCGGCCGCTGAGAAAAAGCGAAGCGGCACTGCTCTTT | |
| | AACAATTTATCAGACAATCTGTGTGGGCACTCGAAGATACGGATTCTTAACGTCGCAAGAC | |
| | GAAAAATGAATACCAAGTCTCAAGAGTGAACACGTAATTCATTACGAAGTTTAATTCTTTG | |
| | AGCGTCAAACTTTTAAATTGAAGAGTTTGATCATGGCTCAGATTGAACGCTGGCGGCAGGC | |
| | CTAACACATGCAAGTCGAACGGTAACAGGAAGAAGCTTGCTTCTTTGCTGACGAGTGGCGG | |
| | ACGGGTGAGTAATGTCTGGGAAACTGCCTGATGGAGGGGGATAACTACTGGAAACGGTAGC | |
| | TAATACCGCATAACGTCGCAAGACCAAAGAGGGGGACCTTCGGGCCTCTTGCCATCGGATG | |
| | TGCCCAGATGGGATTAGCTAGTAGGTGGGGTAACGGCTCACCTAGGCGACGATCCCTAGCT | |
| | GGTCTGAGAGGATGACCAGCCACACTGGAACTGAGACACGGTCCAGACTCCTACGGGAGGC | |
| | AGCAGTGGGGAATATTGCACAATGGGCGCAAGCCTGATGCAGCCATGCCGCGTGTATGAAG | |
| | AAGGCCTTCGGGTTGTAAAGTACTTTCAGCGGGGAGGAAGGGAGTAAAGTTAATACCTTTG | |
| | CTCATTGACGTTACCCGCAGAAGAAGCACCGGCTAACTCCGTGCCAGCAGCCGCGGTAATA | |
| | CGGAGGGTGCAAGCGTTAATCGGAATTACTGGGCGTAAAGCGCACGCAGGCGGTTTGTTAA | |
| | GTCAGATGTGAAATCCCCGGGCTCAACCTGGGAACTGCATCTGATACTGGCAAGCTTGAGT | |
| | CTCGTAGAGGGGGGTAGAATTCCAGGTGTAGCGGTGAAATGCGTAGAGATCTGGAGGAATA | |
| | CCGGTGGCGAAGGCGGCCCCCTGGACGAAGACTGACGCTCAGGTGCGAAAGCGTGGGGAGC | |
| | AAACAGGATTAGATACCCTGGTAGTCCACGCCGTAAACGATGTCGACTTGGAGCTTGATTC | |
| | CGGAGCTAACGCGTTAAGTCGACCGCCTGGGGAGTACGGCCGCAAGGTTAAAACTCAAATG | |
| | AATTGACGGGGGCCCGCACAAGCGGTGGAGCATGTGGTTTAATTCGATGCAACGCGAAGAA | |
| | CCTTACCTGGTCTTGACATCCACGGAAGTTTTCAGAGATGAGAATGTGCCTTCGGGAACCG | |
| | TGAGACAGGTGCTGCATGGCTGTCGTCAGCTCGTGTTGTGAAATGTTGGGTTAAGTCCCGC | |
| | AACGAGCGCAACCCTTATCCTTTGTTGCCAGCGGTCCGGCCGGGAACTCAAAGGAGACTGC | |
| | CAGTGATAAACTGGAGGAAGGTGGGGATGACGTCAAGTCATCATGGCCCTTACGACCAGGG | |
| | CTACACACGTGCTACAATGGCGCATACAAAGAGAGACCTCATAAAGTGCGTCGTAGTCCGG | |
| | ATTGGAGTCTGCAACTCGACTCCATGAAGTCGGAATCGCTAGTAATCGTGGATCAGAATGC | |
| | CACGGTGAATACGTTCCCGGGCCTTGTACACACCGCCCGTCACACCATGGGAGTGGGTTGC | |
| | AAAAGAAGTAGGTAGCTTAACCTTCGGGAGGGCGCTTACCACTTTGTGATTCATGACTGGG | |
| | GTGAAGTCGTAACAAGGTAACCGTAGGGGAACCTGCGGTTGGATCAtgtggtTACCTTAAA | |
| | GAAGCGTACTTTGTAGTGCTCACACAGATTGTCTGATAGAAAGTGAAAAGCAAGGCGTTTA | |
| | CGCGTTGGGAGTGAGGCTGAAGAGAATAAGGCCGTTCGCTTTCTATTAATGAAAGCTCACC | |
| | CTACACGAAAATATCACGCAACGCGTGATAAGCAATTTTCGTGTCCCCTTCGTCTAGAGGC | |
| | CCAGGACACCGCCCTTTCACGGCGGTAACAGGGGTTCGAATCCCCTAGGGGACGCCACTTG | |
| | CTGGTTTGTGAGTGAAAGTCGCCGACCTTAATATCTCAAAACTCATCTTCGGGTGATGTTT | |
| | GAGATATTTGCTCTTTAAAAATCTGGATCAAGCTGAAAATTGAAACACTGAACAACGAGAG | |
| | TTGTTCGTGAGTCTCTCAAATTTTCGCAACACGATGATGAATCGAAAGAAACATCTTCGGG | |
| | TTGTGAGGTTAAGCGACTAAGCGTACACGGTGGATGCCCTGGCAGTCAGAGGCGATGAAGG | |
| | ACGTGCTAATCTGCGATAAGCGTCGGTAAGGTGATATGAACCGTTATAACCGGCGATTTCC | |
| | GAATGGGGAAACCCAGTGTGTTTCGACACACTATCATTAACTGAATCCATAGGTTAATGAG | |
| | GCGAACCGGGGGAACTGAAACATCTAAGTACCCCGAGGAAAAGAAATCAACCGAGATTCCC | |
| | CCAGTAGCGGCGAGCGAACGGGGAGCAGCCCAGAGCCTGAATCAGTGTGTGTGTTAGTGGA | |
| | AGCGTCTGGAAAGGCGCGCGATACAGGGTGACAGCCCCGTACACAAAAATGCACATGCTGT | |
| | GAGCTCGATGAGTAGGGCGGGACACGTGGTATCCTGTCTGAATATGGGGGGACCATCCTCC | |
| | AAGGCTAAATACTCCTGACTGACCGATAGTGAACCAGTACCGTGAGGGAAAGGCGAAAAGA | |
| | ACCCCGGCGAGGGGAGTGAAAAAGAACCTGAAACCGTGTACGTACAAGCAGTGGGAGCACG | |
| | CTTAGGCGTGTGACTGCGTACCTTTTGTATAATGGGTCAGCGACTTATATTCTGTAGCAAG | |
| | GTTAACCGAATAGGGGAGCCGAAGGGAAACCGAGTCTTAACTGGGCGTTAAGTTGCAGGGT | |
| | ATAGACCCGAAACCCGGTGATCTAGCCATGGGCAGGTTGAAGGTTGGGTAACACTAACTGG | |
| | AGGACCGAACCGACTAATGTTGAAAAATTAGCGGATGACTTGTGGCTGGGGGTGAAAGGCC | |
| | AATCAAACCGGGAGATAGCTGGTTCTCCCCGAAAGCTATTTAGGTAGCGCCTCGTGAATTC | |
| | ATCTCCGGGGGTAGAGCACTGTTTCGGCAAGGGGGTCATCCCGACTTACCAACCCGATGCA | |
| | AACTGCGAATACCGGAGAATGTTATCACGGGAGACACACGGCGGGTGCTAACGTCCGTCGT | |
| | GAAGAGGGAAACAACCCAGACCGCCAGCTAAGGTCCCAAAGTCATGGTTAAGTGGGAAACG | |
| | ATGTGGGAAGGCCCAGACAGCCAGGATGTTGGCTTAGAAGCAGCCATCATTTAAAGAAAGC | |
| | GTAATAGCTCACTGGTCGAGTCGGCCTGCGCGGAAGATGTAACGGGGCTAAACCATGCACC | |
| | GAAGCTGCGGCAGCGACGCTTATGCGTTGTTGGGTAGGGGAGCGTTCTGTAAGCCTGCGAA | |
| | GGTGTGCTGTGAGGCATGCTGGAGGTATCAGAAGTGCGAATGCTGACATAAGTAACGATAA | |
| | AGCGGGTGAAAAGCCCGCTCGCCGGAAGACCAAGGGTTCCTGTCCAACGTTAATCGGGGCA | |
| | GGGTGAGTCGACCCCTAAGGCGAGGCCGAAAGGCGTAGTCGATGGGAAACAGGTTAATATT | |
| | CCTGTACTTGGTGTTACTGCGAAGGGGGGACGGAGAAGGCTATGTTGGCCGGGCGACGGTT | |
| | GTCCCGGTTTAAGCGTGTAGGCTGGTTTTCCAGGCAAATCCGGAAAATCAAGGCTGAGGCG | |
| | TGATGACGAGGCACTACGGTGCTGAAGCAACAAATGCCCTGCTTCCAGGAAAAGCCTCTAA | |
| | GCATCAGGTAACATCAAATCGTACCCCAAACCGACACAGGTGGTCAGGTAGAGAATACCAA | |
| | GGCGCTTGAGAGAACTCGGGTGAAGGAACTAGGCAAAATGGTGCCGTAACTTCGGGAGAAG | |
| | GCACGCTGATATGTAGGTGAGGTCCCTCGCGGATGGAGCTGAAATCAGTCGAAGATACCAG | |
| | CTGGCTGCAACTGTTTATTAAAAACACAGCACTGTGCAAACACGAAAGTGGACGTATACGG | |
| | TGTGACGCCTGCCCGGTGCCGGAAGGTTAATTGATGGGGTTAGCGCAAGCGAAGCTCTTGA | |
| | TCGAAGCCCCGGTAAACGGCGGCCGTAACTATAACGGTCCTAAGGTAGCGAAATTCCTTGT | |
| | CGGGTAAGTTCCGACCTGCACGAATGGCGTAATGATGGCCAGGCTGTCTCCACCCGAGACT | |
| | CAGTGAAATTGAACTCGCTGTGAAGATGCAGTGTACCCGCGGCAAGACGGAAAGACCCCGT | |
| | GAACCTTTACTATAGCTTGACACTGAACATTGAGCCTTGATGTGTAGGATAGGTGGGAGGC | |
| | TTTGAAGTGTGGACGCCAGTCTGCATGGAGCCGACCTTGAAATACCACCCTTTAATGTTTG | |
| | ATGTTCTAACGTTGACCCGTAATCCGGGTTGCGGACAGTGTCTGGTGGGTAGTTTGACTGG | |
| | GGCGGTCTCCTCCTAAAGAGTAACGGAGGAGCACGAAGGTTGGCTAATCCTGGTCGGACAT | |
| | CAGGAGGTTAGTGCAATGGCATAAGCCAGCTTGACTGCGAGCGTGACGGCGCGAGCAGGTG | |
| | CGAAAGCAGGTCATAGTGATCCGGTGGTTCTGAATGGAAGGGCCATCGCTCAACGGATAAA | |
| | AGGTACTCCGGGGATAACAGGCTGATACCGCCCAAGAGTTCATATCGACGGCGGTGTTTGG | |
| | CACCTCGATGTCGGCTCATCACATCCTGGGGCTGAAGTAGGTCCCAAGGGTATGGCTGTTC | |
| | GCCATTTAAAGTGGTACGCGAGCTGGGTTTAGAACGTCGTGAGACAGTTCGGTCCCTATCT | |
| | GCCGTGGGCGCTGGAGAACTGAGGGGGGCTGCTCCTAGTACGAGAGGACCGGAGTGGACGC | |
| | ATCACTGGTGTTCGGGTTGTCATGCCAATGGCACTGCCCGGTAGCTAAATGCGGAAGAGAT | |
| | AAGTGCTGAAAGCATCTAAGCACGAAACTTGCCCCGAGATGAGTTCTCCCTGACCCTTTAA | |
| | GGGTCCTGAAGGAACGTTGAAGACGACGACGTTGATAGGCCGGGTGTGTAAGCGCAGCGAT | |
| | GCGTTGAGCTAACCGGTACTAATGAACCGTGAGGCTTAACCTTACAACGCCGAAGCTGTTT | |
| | TGGCGGATGAGAGAAGATTTTCAGCCTGATACAGATTAAATCAGAACGCAGAAGCGGTCTG | |
| | ATAAAACAGAATTTGCCTGGCGGCAGTAGCGCGGTGGTCCCACCTGACCCCATGCCGAACT | |
| | CAGAAGTGAAACGCCGTAGCGCCGATGGTAGTGTGGGGTCTCCCCATGCGAGAGTAGGGAA | |
| | CTGCCAGGCATCAAATAAAACGAAAGGCTCAGTCGAAAGACTGGGCCTTTCGTTTTATCTG | |
| | TTGTTTGTCGGTGAACGCTCTCCTGAGTAGGACAAATCCGCCGGGAGCGGATTTGAACGTT | |
| | GCGAAGCAACGGCCCGGAGGGTGGCGGGCAGGACGCCCGCCATAAACTGCCAGGCATCAAA | |
| | TTAAGCAGAAGGCCATCCTGACGGATGGCCTTTTTGCGTTTCTACAAACTCTTCCTGTCGT | |
| | CATATCTACAAGCCGGCGCGCCGGGAAATGTGCGCGGAACCCCTATTTGTTTATTTTTCTA | |
| | AATACATTCAAATATGTATCCGCTCATGAGACAATAACCCTGATAAATGCTTCAATAATAT | |
| | TGAAAAAGGAAGAGTATGAGTATTCAACATTTCCGTGTCGCCCTTATTCCCTTTTTTGCGG | |
| | CATTTTGCCTTCCTGTTTTTGCTCACCCAGAAACGCTGGTGAAAGTAAAAGATGCTGAAGA | |
| | TCAGTTGGGTGCACGAGTGGGTTACATCGAACTGGATCTCAACAGCGGTAAGATCCTTGAG | |
| | AGTTTTCGCCCCGAAGAACGTTTTCCAATGATGAGCACTTTTAAAGTTCTGCTATGTGGCG | |
| | CGGTATTATCCCGTGTTGACGCCGGGCAAGAGCAACTCGGTCGCCGCATACACTATTCTCA | |
| | GAATGACTTGGTTGAGTACTCACCAGTCACAGAAAAGCATCTTACGGATGGCATGACAGTA | |
| | AGAGAATTATGCAGTGCTGCAATAACCATGAGTGATAACACTGCGGCCAACTTACTTCTGA | |
| | CAACGATCGGAGGACCGAAGGAGCTAACCGCTTTTTTGCACAACATGGGGGATCATGTAAC | |
| | TCGCCTTGATCGTTGGGAACCGGAGCTGAATGAAGCCATACCAAACGACGAGCGTGACACC | |
| | ACGATGCCTGCAGCAATGGCAACAACGTTGCGCAAACTATTAACTGGCGAACTACTTACTC | |
| | TAGCTTCCCGGCAACAATTAATAGACTGGATGGAGGCGGATAAAGTTGCAGGACCACTTCT | |
| | GCGCTCGGCCCTTCCGGCTAGCTGGTTTATTGCTGATAAATCTGGAGCCGGTGAGCGTGGG | |
| | TCTCGCGGTATCATTGCAGCACTGGGGCCAGATGGTAAGCCCTCCCGTATCGTAGTTATCT | |
| | ACACGACGGGGAGTCAGGCAACTATGGATGAACGAAATAGACAGATCGCTGAGATAGGTGC | |
| | CTCACTGATTAAGCATTGGTAACTGCAGACCAAGTTTACTCATATATACTTTAGATTGATT | |
| | TAAAACTTCATTTTTAATTTAAAAGGATCTAGGTGAAGATCCTTTTTGATAATCTCATGAC | |
| | CAAAATCCCTTAACGTGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCAAACAAAAAAACCAC | |
| | CGCTACCAGCGGTGGTTTGTTTGCCGGATCAAGAGCTACCAACTCTTTTTCCGAAGGTAAC | |
| | TGGCTTCAGCAGAGCGCAGATACCAAATACTGTCCTTCTAGTGTAGCCGTAGTTAGGCCAC | |
| | CACTTCAAGAACTCTGTAGCACCGCCTACATACCTCGCTCTGCTAATCCTGTTACCAGTGG | |
| | CTGCTGCCAGTGGCGATAAGTCGTGTCTTACCGGGTTGGACTCAAGACGATAGTTACCGGA | |
| | TAAGGCGCAGCGGTCGGGCTGAACGGGGGGTTCGTGCACACAGCCCAGCTTGGAGCGAACG | |
| | ACCTACACCGAACTGAGATACCTACAGCGTGAGCTATGAGAAAGCGCCACGCTTCCCGAAG | |
| | GGAGAAAGGCGGACAGGTATCCGGTAAGCGGCAGGGTCGGAACAGGAGAGCGCACGAGGGA | |
| | GCTTCCAGGGGGAAACGCCTGGTATCTTTATAGTCCTGTCGGGTTTCGCCACCTCTGACTT | |
| | GAGCGTCGATTTTTGTGATGCTCGTCAGGGGGGCGGAGCCTATGGAAAAACGCCAGCAACG | |
| | CGGCCTTTTTACGGTTCCTGGCCTTTTGCTGG | |
| dT7RNAP | aacacgattaacatcgctaagaacgacttctctgacatcgaactggctgctatcccgttca | 114 |
| sequence | acactctggctgaccattacggtgagcgtttagctcgcgaacagttggcccttgagcatga | |
| | gtcttacgagatgggtgaagcacgcttccgcaagatgtttgagcgtcaacttaaagctggt | |
| | gaggttgcggataacgctgccgccaagcctctcatcactaccctactccctaagatgattg | |
| | cacgcatcaacgactggtttgaggaagtgaaagctaagcgcggcaagcgcccgacagcctt | |
| | ccagttcctgcaagaaatcaagccggaagccgtagcgtacatcaccattaagaccactctg | |
| | gcttgcctaaccagtgctgacaatacaaccgttcaggctgtagcaagcgcaatcggtcggg | |
| | ccattgaggacgaggctcgcttcggtcgtatccgtgaccttgaagctaagcacttcaagaa | |
| | aaacgttgaggaacaactcaacaagcgcgtagggcacgtctacaagaaagcatttatgcaa | |
| | gttgtcgaggctgacatgctctctaagggtctactcggtggcgaggcgtggtcttcgtggc | |
| | ataaggaagactctattcatgtaggagtacgctgcatcgagatgctcattgagtcaaccgg | |
| | aatggttagcttacaccgccaaaatgctggcgtagtaggtcaagactctgagactatcgaa | |
| | ctcgcacctgaatacgctgaggctatcgcaacccgtgcaggtgcgctggctggcatctctc | |
| | cgatgttccaaccttgcgtagttcctcctaagccgtggactggcattactggtggtggcta | |
| | ttgggctaacggtcgtcgtcctctggcgctggtgcgtactcacagtaagaaagcactgatg | |
| | cgctacgaagacgtttacatgcctgaggtgtacaaagcgattaacattgcgcaaaacaccg | |
| | catggaaaatcaacaagaaagtcctagcggtcgccaacgtaatcaccaagtggaagcattg | |
| | tccggtcgaggacatccctgcgattgagcgtgaagaactcccgatgaaaccggaagacatc | |
| | gacatgaatcctgaggctctcaccgcgtggaaacgtgctgccgctgctgtgtaccgcaagg | |
| | acaaggctcgcaagtctcgccgtatcagccttgagttcatgcttgagcaagccaataagtt | |
| | tgctaaccataaggccatctggttcccttacaacatggactggcgcggtcgtgtttacgct | |
| | gtgtcaatgttcaacccgcaaggtaacgatatgaccaaaggactgcttacgctggcgaaag | |
| | gtaaaccaatcggtaaggaaggttactactggctgaaaatccacggtgcaaactgtgcggg | |
| | tgtcgataaggttccgttccctgagcgcatcaagttcattgaggaaaaccacgagaacatc | |
| | atggcttgcgctaagtctccactggagaacacttggtgggctgagcaagattctccgttct | |
| | gcttccttgcgttctgctttgagtacgctggggtacagcaccacggcctgagctataactg | |
| | ctcccttccgctggcgtttgacgggtcttgctctggcatccagcacttctccgcgatgctc | |
| | cgagatgaggtaggtggtcgcgcggttaacttgcttcctagtgaaaccgttcaggacatct | |
| | acgggattgttgctaagaaagtcaacgagattctacaagcagacgcaatcaatgggaccga | |
| | taacgaagtagttaccgtgaccgatgagaacactggtgaaatctctgagaaagtcaagctg | |
| | ggcactaaggcactggctggtcaatggctggcttacggtgttactcgcagtgtgactaagc | |
| | gttcagtcatgacgctggcttacgggtccaaagagttcggcttccgtcaacaagtgctgga | |
| | agataccattcagccagctattgattccggcaagggtctgatgttcactcagccgaatcag | |
| | gctgctggatacatggctaagctgatttgggaatctgtgagcgtgacggtggtagctgcgg | |
| | ttgaagcaatgaactggcttaagtctgctgctaagctgctggctgctgaggtcaaagataa | |
| | gaagactggagagattcttcgcaagcgttgcgctgtgcattgggtaactcctgatggtttc | |
| | cctgtgtggcaggaatacaagaagcctattcagacgcgcttgaacctgatgttcctcggtc | |
| | agttccgcttacagcctaccattaacaccaacaaagatagcgagattgatgcacacaaaca | |
| | ggagtctggtatcgctcctaactttgtacacagccaagacggtagccaccttcgtaagact | |
| | gtagtgtgggcacacgagaagtacggaatcgaatcttttgcactgattcacgGctccttcg | |
| | gtaccattccggctgacgctgcgaacctgttcaaagcagtgcgcgaaactatggttgacac | |
| | atatgagtcttgtgatgtactggctgatttctacgaccagttcgctgaccagttgcacgag | |
| | tctcaattggacaaaatgccagcacttccggctaaaggtaacttgaacctccgtgacatct | |
| | tagagtcggacttcgcgttcgcg | |

It is contemplated that variant or mutant forms of the sequences presented herein can also be employed in making and using nucleotide and/or protein constructs of the disclosure. Accordingly, the exemplary sequences presented herein can be modified to contain one, two, three, four, five or more variant residues, as compared to those disclosed herein, and still remain within the scope of the contemplated disclosure. Similarly, it is contemplated that a sequence at least 80% identical, at least 90% identical, at least 95% identical, at least 97% identical, at least 98% identical or at least 99% identical to one or more of the specific sequences recited herein can be employed in the compositions and methods of the instant disclosure.

### REFERENCES

Agarwal, D., Gregory, S. T. and O'Connor, M. (2011) ‘Error-prone and error-restrictive mutations affecting ribosomal protein S12’, Journal of molecular biology. 410 (1), pp. 1-9.
Agarwal, D., Kamath, D., Gregory, S. T. and O'Connor, M. (2015) ‘Modulation of decoding fidelity by ribosomal proteins S4 and S5’, Journal of bacteriology. 197 (6), pp. 1017-1025.
Aleksashin, N. A., Leppik, M., Hockenberry, A. J., Klepacki, D., Vazquez-Laslop, N., Jewett, M. C., Remme, J. and Mankin, A. S. (2019) ‘Assembly and functionality of the ribosome with tethered subunits’, Nature communications. 10 (1), pp. 930.
Aleksashin, N. A., Szal, T., D'Aquino, A. E., Jewett, M. C., Vazquez-Laslop, N. and Mankin, A. S. (2020) ‘A fully orthogonal system for protein synthesis in bacterial cells’, NatureCommunications. 11 (1).
Alksne, L. E., Anthony, R. A., Liebman, S. W. and Warner, J. R. (1993) ‘An accuracy center in the ribosome conserved over 2 billion years’, Proc Natl Acad Sci 90 (20), pp. 9538-9541.
An, W., and Chin, J. W. (2009). Synthesis of orthogonal transcription-translation networks. Proc Natl Acad Sci 106, 8477-8482.
Asai, T., Condon, C., Voulgaris, J., Zaporojets, D., Shen, B., Al-Omar, M., Squires, C. and Squires, C. L. (1999a) ‘Construction and initial characterization of Escherichia coli strains with few or no intact chromosomal rRNA operons’, Journal of bacteriology. 181 (12), pp. 3803-3809.
Asai, T., Zaporojets, D., Squires, C. and Squires, C. L. (1999b) ‘An Escherichia coli strain with all chromosomal rRNA operons inactivated: complete exchange of rRNA genes between bacteria’, Proceedings of the National Academy of Sciences. 96 (5), pp. 1971-1976.
Badran, A. H., Guzov, V. M., Huai, Q., Kemp, M. M., Vishwanath, P., Kain, W., Nance, A. M., Evdokimov, A., Moshiri, F., Turner, K. H., Wang, P., Malvar, T. and Liu, D. R. (2016) ‘Continuous evolution of Bacillus thuringiensis toxins overcomes insect resistance’, Nature. 533 (7601), pp. 58.
Badran, A. H. and Liu, D. R. (2015a) ‘Development of potent in vivo mutagenesis plasmids with broad mutational spectra’, Nature communications. 6, pp. 8425.
Badran, A. H. and Liu, D. R. (2015b) ‘Development of potent in vivo mutagenesis plasmids with broad mutational spectra’, Nat Commun. 6, pp. 8425.
Badran, A. H. and Liu, D. R. (2015c) ‘In vivo continuous directed evolution’, Current opinion in chemical biology. 24, pp. 1-10.
Basan, M., Zhu, M., Dai, X., Warren, M., Sevin, D., Wang, Y. P. and Hwa, T. (2015) ‘Inflating bacterial cells by increased protein synthesis’, Mol Syst Biol. 11 (10), pp. 836.
Bennett, N. J. and Rakonjac, J. (2006) ‘Unlocking of the filamentous bacteriophage virion during infection is mediated by the C domain of pIII’, J Mol Biol. 356 (2), pp. 266-73.
Bernier, C. R., Petrov, A. S., Waterbury, C. C., Jett, J., Li, F., Freil, L. E., Xiong, X., Wang, L., Migliozzi, B. L., Hershkovits, E., Xue, Y., Hsiao, C., Bowman, J. C., Harvey, S. C., Grover, M. A., Wartell, Z. J. and Williams, L. D. (2014) ‘Ribo Vision suite for visualization and analysis of ribosomes’, Faraday Discuss. 169, pp. 195-207.
Bjorkman, J., Samuelsson, P., Andersson, D. I. and Hughes, D. (1999) ‘Novel ribosomal mutations affecting translational accuracy, antibiotic resistance and virulence of Salmonella typhimurium’, Molecular Microbiology. 31 (1), pp. 53-58.
Boel, G., Letso, R., Neely, H., Price, W. N., Wong, K.-H., Su, M., Luff, J. D., Valecha, M., Everett, J. K., Acton, T. B., Xiao, R., Montelione, G. T., Aalberts, D. P. and Hunt, J. F. (2016) ‘Codon influence on protein expression in E. coli correlates with mRNA levels’, Nature. 529 (7586), pp. 358-363.
Bratulic, S. and Badran, A. H. (2017) ‘Modern methods for laboratory diversification of biomolecules’, Current Opinion in Chemical Biology. 41, pp. 50-60.
Brodersen, D. E., Clemons Jr, W. M., Carter, A. P., Wimberly, B. T. and Ramakrishnan, V. (2002) ‘Crystal structure of the 30 S ribosomal subunit from Thermus thermophilus: structure of 35 the proteins and their interactions with 16 S RNA’, Journal of molecular biology. 316 (3), pp. 725-768.
Brosius, J., Dull, T. J., Sleeter, D. D., and Noller, H. F. (1981). Gene organization and primary structure of a ribosomal RNA operon from Escherichia coli. J Mol Biol 148:107-127.
Bryson, D. I., Fan, C., Guo, L. T., Miller, C., Soll, D. and Liu, D. R. (2017) ‘Continuous directed evolution of aminoacyl-tRNA synthetases’, Nat Chem Biol. 13 (12), pp. 1253-1260.
Carlson, J. C., Badran, A. H., Guggiana-Nilo, D. A. and Liu, D. R. (2014a) ‘Negative selection and stringency modulation in phage-assisted continuous evolution’, Nat Chem Biol. 10 (3), pp. 216-22.
Carlson, J. C., Badran, A. H., Guggiana-Nilo, D. A. and Liu, D. R. (2014b) ‘Negative selection and stringency modulation in phage-assisted continuous evolution’, Nature chemical biology. 10 (3), pp. 216.
Chatterjee, A., Lajoie, M. J., Xiao, H., Church, G. M. and Schultz, P. G. (2014) ‘A Bacterial Strain with a Unique Quadruplet Codon Specifying Non-native Amino Acids’, ChemBioChem. 15 (12), pp. 1782-1786.
Cologgi, D. L., Lampa-Pastirk, S., Speers, A. M., Kelly, S. D. and Reguera, G. (2011) ‘Extracellular reduction of uranium via Geobacter conductive pili as a protective cellular mechanism’, Proceedings of the National Academy of Sciences. 108 (37), pp. 15248-15252.
Darlington, A. P., Kim, J., Jimenez, J. I. and Bates, D. G. (2018) ‘Dynamic allocation of orthogonal ribosomes facilitates uncoupling of co-expressed genes’, Nature communications. 9 (1), pp. 695.
Davis, J. H., Rubin, A. J. and Sauer, R. T. (2010) ‘Design, construction and characterization of a set of insulated bacterial promoters’, Nucleic acids research. 39 (3), pp. 1131-1141.
Dennis, P. P., Ehrenberg, M. and Bremer, H. (2004) ‘Control of rRNA Synthesis in Escherichia coli: a Systems Biology Approach’, Microbiology and Molecular Biology Reviews. 68 (4), pp. 639-668.
Esvelt, K. M., Carlson, J. C. and Liu, D. R. (2011) ‘A system for the continuous directed evolution of biomolecules’, Nature. 472 (7344), pp. 499.
Hatzenpichler, R., Scheller, S., Tavormina, P. L., Babin, B. M., Tirrell, D. A. and Orphan, V. J. (2014) ‘In situ visualization of newly synthesized proteins in environmental microbes using amino acid tagging and click chemistry’, Environmental Microbiology. 16 (8), pp. 2568-2590.
Hubbard, B. P., Badran, A. H., Zuris, J. A., Guilinger, J. P., Davis, K. M., Chen, L., Tsai, S. Q., Sander, J. D., Joung, J. K. and Liu, D. R. (2015) ‘Continuous directed evolution of DNA-binding proteins to improve TALEN specificity’, Nature methods. 12 (10), pp. 939.
Hui, S., Silverman, J. M., Chen, S. S., Erickson, D. W., Basan, M., Wang, J., Hwa, T. and Williamson, J. R. (2015) ‘Quantitative proteomic analysis reveals a simple strategy of global resource allocation in bacteria’, Molecular Systems Biology. 11 (2), pp. 784.
Killmann, H., Videnov, G., Jung, G., Schwarz, H. and Braun, V. (1995) ‘Identification of receptor binding sites by competitive peptide mapping: phages T1, T5, and phi 80 and colicin M bind to the gating loop of FhuA’, Journal of bacteriology. 177 (3), pp. 694-698.
Klumpp, S., Scott, M., Pedersen, S. and Hwa, T. (2013) ‘Molecular crowding limits translation and cell growth’, Proceedings of the National Academy of Sciences. 110 (42), pp. 16754-16759.
Kolber, N. S., Fattal, R., Bratulic, S., Carver, G. D. and Badran, A. H. (2021) ‘Orthogonal translation enables heterologous ribosome engineering in E. coli’, Nat Commun. 12 (1), pp. 599.
Kolber, N. S., Fattal R., Bratulic, S., & Badran A. H. (2020) ‘Orthogonal Translation Enables Heterologous Ribosome Engineering in E. coli’, In revision. Liu, C. C., Jewett, M. C., Chin, J. W. and Voigt, C. A. (2018) ‘Toward an orthogonal central dogma’, Nature chemical biology. 14 (2), pp. 103.
Lund, A. M., Kildegaard, H. F., Petersen, M. B. K., Rank, J., Hansen, B. G., Andersen, M. R. and Mortensen, U. H. (2014) ‘A versatile system for USER cloning-based assembly of expression vectors for mammalian cell engineering’, PloS one. 9 (5), pp. e96693.
McClory, S. P., Devaraj, A. and Fredrick, K. (2014) ‘Distinct functional classes of ram mutations in 16S rRNA’, RNA. 20 (4), pp. 496-504.
McDowell, J. C., Roberts, J. W., Jin, D. J., and Gross, C. (1994). Determination of intrinsic transcription termination efficiency by RNA polymerase elongation rate. Science 266, 822-825.
Moll, I., Grill, S., Gründling, A. and Blasi, U. (2002) ‘Effects of ribosomal proteins S1, S2 and the DeaD/CsdA DEAD-box helicase on translation of leaderless and canonical mRNAs in Escherichia coli’, Molecular microbiology. 44 (5), pp. 1387-1396.
Mordret, E., Dahan, O., Asraf, O., Rak, R., Yehonadav, A., Barnabas, G. D., Cox, J., Geiger, T., Lindner, A. B. and Pilpel, Y. (2019) ‘Systematic Detection of Amino Acid Substitutions in Proteomes Reveals Mechanistic Basis of Ribosome Errors and Selection for Translation Fidelity’, Molecular Cell. 75 (3), pp. 427-441.e5.
Neumann, H., Wang, K., Davis, L., Garcia-Alai, M. and Chin, J. W. (2010) ‘Encoding multiple unnatural amino acids via evolution of a quadruplet-decoding ribosome’, Nature. 464 (7287), pp. 441.
Novoa, E. M., Pavon-Eternod, M., Pan, T. and Ribas de Pouplana, L. (2012) ‘A role for tRNA modifications in genome structure and codon usage’, Cell. 149 (1), pp. 202-13.
Orelle, C., Carlson, E. D., Szal, T., Florin, T., Jewett, M. C. and Mankin, A. S. (2015) ‘Protein synthesis by ribosomes with tethered subunits’, Nature. 524 (7563), pp. 119.
O'Connor, M. and Dahlberg, A. E. (2001) ‘Enhancement of translation by the epsilon element is independent of the sequence of the 460 region of 16S rRNA’, Nucleic acids research. 29 (7), pp. 1420-1425.
Peterson, J. and Phillips, G. J. (2008) ‘New pSC101-derivative cloning vectors with elevated copy numbers’, Plasmid. 59 (3), pp. 193-201.
Polikanov, Y. S., Blaha, G. M. and Steitz, T. A. (2012) ‘How Hibernation Factors RMF, HPF, and YfiA Turn Off Protein Synthesis’, Science. 336 (6083), pp. 915-918.
Rackham, O. and Chin, J. W. (2005) ‘A network of orthogonal ribosome· mRNA pairs’, Nature chemical biology. 1 (3), pp. 159.
Recht, M. I. and Puglisi, J. D. (2001) ‘Aminoglycoside Resistance with Homogeneous and Heterogeneous Populations of Antibiotic-Resistant Ribosomes’, Antimicrobial Agents and Chemotherapy. 45 (9), pp. 2414-2419.
Reynolds, R., Bermúdez-Cruz, R. M., and Chamberlin, M. J. (1992). Parameters affecting transcription termination by Escherichia coli RNA polymerase: I. Analysis of 13 rho-independent terminators. J Mol Biol 224, 31-51.
Riba, A., Di Nanni, N., Mittal, N., Arhne, E., Schmidt, A. and Zavolan, M. (2019) ‘Protein synthesis rates and ribosome occupancies reveal determinants of translation elongation rates’, Proceedings of the National Academy of Sciences. 116 (30), pp. 15023-15032.
Roller, B. R., Stoddard, S. F. and Schmidt, T. M. (2016) ‘Exploiting rRNA operon copy number to investigate bacterial reproductive strategies’, Nat Microbiol. 1 (11), pp. 16160.
Saito, K., Green, R. and Buskirk, A. R. (2020) ‘Translational initiation in E. coli occurs at the correct sites genome-wide in the absence of mRNA-rRNA base-pairing’, eLife. 9.
Scott, M., Klumpp, S., Mateescu, E. M. and Hwa, T. (2014) ‘Emergence of robust growth laws from optimal regulation of ribosome synthesis’, Molecular Systems Biology. 10 (8), pp. 747.
Selmer, M., Dunham, C. M., Murphy, F. V., Weixlbaumer, A., Petry, S., Kelley, A. C., Weir, J. R. and Ramakrishnan, V. (2006) ‘Structure of the 70S ribosome complexed with mRNA and RNA’, Science. 313 (5795), pp. 1935-1942.
Serbanescu, D., Ojkic, N. and Banerjee, S. (2020) ‘Nutrient-Dependent Trade-Offs between Ribosomes and Division Protein Synthesis Control Bacterial Cell Size and Growth’, Cell Rep. 32 (12), pp. 108183.
Soisson, S. M., MacDougall-Shackleton, B., Schleif, R. and Wolberger, C. (1997) ‘The 1.6 A crystal structure of the AraC sugar-binding and dimerization domain complexed with D-fucose’, Journal of molecular biology. 273 (1), pp. 226-237.
Sprouffske, K. (Jul. 30, 2018) Using Growthcurver. Available at: cran.rproject.org/web/packages/growthcurver/vignettes/Growthcurver-vignette.html (Accessed: May 2019).
Taheri-Araghi, S., Bradde, S., Sauls, T., John, Hill, S., Norbert, Levin, A., Petra, Paulsson, J., Vergassola, M. and Jun, S. (2015) ‘Cell-Size Control and Homeostasis in Bacteria’, Current Biology. 25 (3), pp. 385-391.
Thuronyi, B. W., Koblan, L. W., Levy, J. M., Yeh, W.-H., Zheng, C., Newby, G. A., Wilson, C., Bhaumik, M., Shubina-Oleinik, O. and Holt, J. R. (2019) ‘Continuous evolution of base editors with expanded target compatibility and improved activity’, Nature Biotechnology, pp. 1.
Vallabhaneni, H. and Farabaugh, P. J. (2009) ‘Accuracy modulating mutations of the ribosomal protein S4-S5 interface do not necessarily destabilize the rps4-rps5 protein-protein interaction’, RNA. 15 (6), pp. 1100-9.
Vila-Sanjurjo, A., Ridgeway, W. K., Seymaner, V., Zhang, W., Santoso, S., Yu, K. and Cate, J. H. D. (2003) ‘X-ray crystal structures of the WT and a hyper-accurate ribosome from Escherichia coli’, Proceedings of the National Academy of Sciences. 100 (15), pp. 8682-8687.
Wang, H. H. and Church, G. M. (2011) ‘Multiplexed genome engineering and genotyping methods: applications for synthetic biology and metabolic engineering’, Methods in enzymology: Elsevier, pp. 409-426.
Wang, T., Badran, A. H., Huang, T. P. and Liu, D. R. (2018) ‘Continuous directed evolution of proteins with improved soluble expression’, Nature chemical biology. 14 (10), pp. 972.
Wilcox, G. (1974) ‘The interaction of L-arabinose and D-fucose with AraC protein’, Journal of Biological Chemistry. 249 (21), pp. 6892-6894.
Zaher, H. S. and Green, R. (2010) ‘Hyperaccurate and error-prone ribosomes exploit distinct mechanisms during tRNA selection’, Mol Cell. 39 (1), pp. 110-20.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the instant description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A synthetic variant 16S ribosomal RNA (rRNA) comprising one or more mutations selected from the group consisting of U409C, C440U, U904C, A906G, C1098U, G1415A and G1487A, wherein residue numbering is relative to the E. coli 16S rRNA sequence of SEQ ID NO: 40.

2. The synthetic variant 16S rRNA of claim 1, wherein the one or more mutations is present in a 16S rRNA sequence of a strain selected from the group consisting of E. coli, S. enterica, C. freundii, K. aerogenes, K. pneumoniae, K. oxytoca, E. cloacae, S. marcescens, P. mirabilis, P. stuartii, V. cholerae, A. macelodii, M. minitulum, P. aeruginosa, A. baumannii, A. faecalis, B. pertussis, B. cenocepacia, N. gonorrhoeae, M. ferrooxydans and C. crescentus.

3. The synthetic variant 16S rRNA of claim 1 comprising U409C and G1487A mutations.

4. A host cell comprising the synthetic variant 16S rRNA sequence of claim 1.

5. A composition selected from among:
A host cell comprising a nucleic acid sequence comprising a non-host cell 16S ribosomal RNA (rRNA) variant sequence comprising one or more mutations selected from the group consisting of U409C, C440U, U904C, A906G, C1098U, G1415A and G1487A, wherein residue numbering is relative to the E. coli 16S rRNA sequence of SEQ ID NO: 40;
A nucleic acid construct comprising an orthogonal anti-ribosome binding site (o-antiRBS) sequence selected from the group consisting of SEQ ID NOs: 8-10;
A nucleic acid construct comprising an orthogonal-ribosome binding site (o-RBS) sequence of SEQ ID NO: 7;
A first nucleic acid construct comprising an orthogonal anti-ribosome binding site (o-antiRBS) sequence selected from the group consisting of SEQ ID NOs: 8-10 and a second nucleic acid construct comprising an orthogonal-ribosome binding site (o-RBS) sequence of SEQ ID NO: 7;
A host cell comprising one or more nucleic acid constructs of SEQ ID NOs: 14-18, optionally wherein at least one of the one or more nucleic acid constructs comprises a non-host cell 16S rRNA sequence;
A host cell comprising a deletion or disruption of ribosome hibernation promoting factor (HPF) in the host cell genome and a nucleic acid sequence comprising a non-host cell ribosomal RNA (rRNA) sequence;
A nucleic acid construct comprising a truncated 16S ribosomal RNA (rRNA);
A nucleic acid construct comprising an orthogonal anti-ribosome binding site (o-antiRBS) and a 16S ribosomal RNA (rRNA) sequence, optionally further comprising 23S and/or 5S ribosomal RNA (rRNA) sequence, optionally further comprising phage genes, optionally wherein the nucleic acid construct comprises a sequence selected from the group consisting of SEQ ID NOs: 85-87;
A nucleic acid construct comprising an orthogonal-ribosome binding site (o-RBS) sequence, an intein sequence, and a gIII sequence, optionally wherein the intein sequence is selected from the group consisting of a GGS2 linker sequence (SEQ ID NO: 83), a MBP sequence (SEQ ID NO: 84) and a dT7RNAP sequence (SEQ ID NO: 114), optionally wherein the nucleic acid construct comprises SEQ ID NO: 93
A host cell comprising a nucleic acid construct comprising an orthogonal anti-ribosome binding site (o-antiRBS) and a 16S ribosomal RNA (rRNA) sequence, optionally further comprising 23S and/or 5S ribosomal RNA (rRNA) sequence, optionally further comprising phage genes, optionally wherein the nucleic acid construct comprises a sequence selected from the group consisting of SEQ ID NOs: 85-87; and/or
A host cell comprising a nucleic acid construct comprising an orthogonal-ribosome binding site (o-RBS) sequence, an intein sequence, and a gIII sequence, optionally wherein the intein sequence is selected from the group consisting of a GGS2 linker sequence (SEQ ID NO: 83), a MBP sequence (SEQ ID NO: 84) and a dT7RNAP sequence (SEQ ID NO: 114), optionally wherein the nucleic acid construct comprises SEQ ID NO: 93.

6. The composition of claim 5, wherein the non-host cell is selected from the group consisting of Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum mimitulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii and Shigella spp. (e.g., Shigella flexneri, Shigella dysenteriae, Shigella sonnei, Shigella boydii).

7. The composition of claim 5, wherein the non-host cell is a commensal microbe, optionally wherein the commensal microbe is of a phylum or phyla selected from the group consisting of Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria, and a combination of phyla thereof.

8. The composition of claim 6, wherein the nucleic acid sequence comprises a non-host cell 16S ribosomal RNA (rRNA) variant sequence further comprises intergenic sequences, optionally wherein the intergenic sequences are host cell intergenic sequences.

9. The composition of claim 6, wherein the non-host cell 16S rRNA variant sequence further comprises an o-antiRBS sequence.

10. The composition of claim 6, further comprising a nucleic acid sequence encoding for S20, S16, S1 and/or S15 r-protein(s) of the non-host cell.

11. The host cell of claim 4, wherein translational output of the host cell comprising the variant 16S rRNA sequence is increased as compared to a control host cell comprising a wild-type 16S rRNA, optionally wherein translational output is increased by at least 10%.

12. The host cell of claim 4, wherein the host cell is Escherichia coli, optionally an E. coli strain comprising a genomic deletion for rRNA sequences, optionally further comprising a counter-selectable plasmid comprising E. coli rRNA sequences, optionally wherein the E. coli strain is selected from the group consisting of S1021, S2057, S2060, S3300, S3301, S3302, S3303, S3314, S3317, S3318, S3319, S3320, S3322, S3485 and S3489.

13. The host cell of claim 4, wherein the host cell is Bacillus subtilis, optionally a B. subtilis strain comprising a genomic deletion for rRNA sequences, optionally further comprising a counter-selectable plasmid comprising B. subtilis rRNA sequence.

14. (canceled)

15. The composition of claim 5, wherein the nucleic acid construct comprises a 16S ribosomal RNA (rRNA) sequence, optionally wherein the nucleic acid construct further comprises 23S and/or 5S ribosomal RNA (rRNA) sequence, optionally wherein the nucleic acid construct further comprises phage genes, optionally wherein the nucleic acid construct comprises a sequence selected from the group consisting of SEQ ID NOs: 97 and 98.

16. (canceled)

17. The composition of claim 5, wherein the nucleic acid construct comprises gIII sequence, optionally wherein the nucleic acid construct comprises a sequence selected from the group consisting of SEQ ID NOs: 89, 91 and 92.

18-20. (canceled)

21. The composition of claim 5, further comprising a synthetic variant 16S ribosomal RNA (rRNA) selected from the group consisting of:
a synthetic variant 16S ribosomal RNA (rRNA) comprising one or more mutations selected from the group consisting of U409C, C440U, U904C, A906G, C1098U, G1415A and G1487A, wherein residue numbering is relative to the E. coli 16S rRNA sequence of SEQ ID NO: 40, optionally wherein the one or more mutations is present in a 16S rRNA sequence of a strain selected from the group consisting of E. coli, S. enterica, C. freundii, K. aerogenes, K. pneumoniae, K. oxytoca, E. cloacae, S. marcescens, P. mirabilis, P. stuartii, V. cholerae, A. macelodii, M. minitulum, P. aeruginosa, A. baumannii, A. faecalis, B. pertussis, B. cenocepacia, N. gonorrhoeae, M. ferrooxydans and C. crescentus; and
a synthetic variant 16S ribosomal RNA (rRNA) comprising U409C and G1487A mutations.

22. The composition of claim 5, wherein the host cell further comprises one or more nucleic acid construct(s) selected from the group consisting of:
a nucleic acid construct comprising an orthogonal anti-ribosome binding site (o-antiRBS) sequence selected from the group consisting of SEQ ID NOs: 8-10, optionally wherein the nucleic acid construct comprises a 16S ribosomal RNA (rRNA) sequence, optionally wherein the nucleic acid construct further comprises 23S and/or 5S ribosomal RNA (rRNA) sequence, optionally wherein the nucleic acid construct further comprises phage genes, optionally wherein the nucleic acid construct comprises a sequence selected from the group consisting of SEQ ID NOs: 97 and 98;
a nucleic acid construct comprising an orthogonal-ribosome binding site (o-RBS) sequence of SEQ ID NO: 7, optionally wherein the nucleic acid construct comprises a gIII sequence, optionally wherein the nucleic acid construct comprises a sequence selected from the group consisting of SEQ ID NOs: 89, 91 and 92;
a first nucleic acid construct comprising an orthogonal anti-ribosome binding site (o-antiRBS) sequence selected from the group consisting of SEQ ID NOs: 8-10 and a second nucleic acid construct comprising an orthogonal-ribosome binding site (o-RBS) sequence of SEQ ID NO: 7; and
SEQ ID NOs: 14-18, optionally wherein at least one of the one or more nucleic acid constructs comprises a non-host cell 16S rRNA sequence of claims 14-19.

23. The composition of claim 5, wherein:
the host cell is Escherichia coli, optionally wherein the E. coli strain is selected from the group consisting of S3300, S3314, S3317, S3322, S3485 and S3489;
propagation of an orthogonal translation system in the host cell is improved by at least 100-fold (optionally by at least 3000-fold), as compared to an appropriate control host cell having genomic ribosome hibernation promoting factor (HPF); and/or
the host cell comprises:
one or more nucleic acid constructs selected from the group consisting of SEQ ID NOs: 89, 91, 92, 97 and 98, optionally wherein the one or more nucleic acid constructs comprise a sequence selected from the group consisting of SEQ ID NOs: 105-113; and/or
a nucleic acid construct comprising a non-host cell truncated 16S ribosomal RNA (rRNA), optionally wherein the nucleic acid construct comprises an E. coli 16S rRNA, optionally wherein the host cell has o-rRNA-mediated translation activity that is retained or enhanced relative to an appropriate control host cell, optionally wherein the host cell possesses o-rRNA-mediated translation activity levels of at least 80% that of an appropriate control host cell (i.e., a corresponding host cell having a full-length 16S o-rRNA).

24-32. (canceled)

33. A method selected from among:
A method for identifying in a host cell a non-host cell ribosomal RNA (rRNA) possessing enhanced translation activity, the method comprising:
(a) performing phage-assisted continuous directed evolution upon a population of host cells, wherein each host cell comprises:
(i) a first nucleic acid construct comprising an orthogonal anti-ribosome binding site (o-antiRBS) and a 16S ribosomal RNA (rRNA) sequence, optionally further comprising 23S and/or 5S ribosomal RNA (rRNA) sequence, optionally further comprising phage genes, optionally wherein the nucleic acid construct comprises a sequence selected from the group consisting of SEQ ID NOs: 85-87; and
(ii) a second nucleic acid construct comprising an orthogonal-ribosome binding site (o-RBS) sequence, an intein sequence, and a gIII sequence, optionally wherein the intein sequence is selected from the group consisting of a GGS2 linker sequence, a maltose binding protein (MBP) sequence and a dT7RNAP sequence, optionally wherein the nucleic acid construct comprises SEQ ID NO: 93;
(b) selecting for host cells comprising increased phage titer, as compared to a starting host cell; and
(c) identifying a non-host cell ribosomal RNA (rRNA) sequence of a selected host cell of step (b),
thereby identifying in a host cell a non-host cell ribosomal RNA (rRNA) possessing enhanced translation activity; and/or
A method for enhancing non-host cell protein synthesis in a host cell, the method comprising introducing a non-host cell translation system comprising a 16S rRNA sequence selected from the group consisting of SEQ ID NOs: 13, 15, 17, 19, 21, 23, 27, 31, 34 and 41-82 to the host cell, wherein non-host cell protein synthesis is enhanced in the host cell relative to an appropriate control (i.e., a host cell having a non-host cell translation system that does not comprise a 16S rRNA sequence of SEQ ID NOs: 13, 15, 17, 19, 21, 23, 27, 31, 34 and 41-82), thereby enhancing non-host cell protein synthesis in the host cell.

34. (canceled)


SEQUENCE LISTING (114 sequences)

SEQ ID NO: 1 - RNA, 20 nt, synthetic construct
  misc_feature 1..20: note = synthetic
  source 1..20: mol_type = other RNA
tttccaaggagggatctatg

SEQ ID NO: 2 - RNA, 13 nt, synthetic construct
  misc_feature 1..13: note = synthetic
  source 1..13: mol_type = other RNA
aattcctccacta

SEQ ID NO: 3 - RNA, 20 nt, synthetic construct
  misc_feature 1..20: note = synthetic
  source 1..20: mol_type = other RNA
tttccaaccacagatctatg

SEQ ID NO: 4 - RNA, 12 nt, synthetic construct
  misc_feature 1..12: note = synthetic
  source 1..12: mol_type = other RNA
attggtgtacta

SEQ ID NO: 5 - RNA, 21 nt, synthetic construct
  misc_feature 1..21: note = synthetic
  variation 7..13: note = n is a, c, g, or u
  source 1..21: mol_type = other RNA
tttccannnnnnngatctatg

SEQ ID NO: 6 - skipped

SEQ ID NO: 7 - RNA, 21 nt, synthetic construct
  misc_feature 1..21: note = synthetic
  source 1..21: mol_type = other RNA
tttccaatatacagatctatg

SEQ ID NO: 8 - RNA, 12 nt, synthetic construct
  misc_feature 1..12: note = synthetic
  source 1..12: mol_type = other RNA
atatatgtacta

SEQ ID NO: 9 - RNA, 12 nt, synthetic construct
  misc_feature 1..12: note = synthetic
  source 1..12: mol_type = other RNA
atatgttcacta

SEQ ID NO: 10 - RNA, 12 nt, synthetic construct
  misc_feature 1..12: note = synthetic
  source 1..12: mol_type = other RNA
attatgtaacta

SEQ ID NO: 11 - DNA, 70 bp, synthetic construct
  misc_feature 1..70: note = synthetic
  source 1..70: mol_type = other DNA
gcagccatgccgcgtgtatgaagaaggccttcgggttgtaaagtactttcagcggggaggaagggagtaa

SEQ ID NO: 12 - DNA, 70 bp, synthetic construct
  misc_feature 1..70: note = synthetic
  source 1..70: mol_type = other DNA
ccagccatgccgcgtgtgtgaagaaggtcttcggattgtaaagcactttaagttgggaggaagggcagta

SEQ ID NO: 13 - DNA, 70 bp, synthetic construct
  misc_feature 1..70: note = synthetic
  source 1..70: mol_type = other DNA
ccagccatgccgcgtgtgtgaagaaggtcttcggattgtaaagcacttttagttgggaggaagggcagta

SEQ ID NO: 14 - DNA, 70 bp, synthetic construct
  misc_feature 1..70: note = synthetic
  source 1..70: mol_type = other DNA
gcagccatgccgcgtgtatgaagaaggccttcgggttgtaaagtactttcagtagggaggaaggtggtta

SEQ ID NO: 15 - DNA, 70 bp, synthetic construct
  misc_feature 1..70: note = synthetic
  source 1..70: mol_type = other DNA
gcagccatgccgcgtgtacgaagaaggccttcgggttgtaaagtactttcagtagggaggaaggtggtta

SEQ ID NO: 16 - DNA, 70 bp, synthetic construct
  misc_feature 1..70: note = synthetic
  source 1..70: mol_type = other DNA
taagtcgaccgcctggggagtacggccgcaaggttaaaactcaaatgaattgacgggggcccgcacaagc

SEQ ID NO: 17 - DNA, 70 bp, synthetic construct
  misc_feature 1..70: note = synthetic
  source 1..70: mol_type = other DNA
taagtcgaccgcctggggagtacggccgcaaggtcagaactcaaatgaattgacgggggcccgcacaagc

SEQ ID NO: 18 - DNA, 70 bp, synthetic construct; identical to SEQ ID NO: 16
  misc_feature 1..70: note = synthetic
  source 1..70: mol_type = other DNA

SEQ ID NO: 19 - DNA, 70 bp, synthetic construct
  misc_feature 1..70: note = synthetic
  source 1..70: mol_type = other DNA
taagtcgaccgcctggggagtacggccgcaaggttagaactcaaatgaattgacgggggcccgcacaagc

SEQ ID NO: 20 - DNA, 70 bp, synthetic construct
  misc_feature 1..70: note = synthetic
  source 1..70: mol_type = other DNA
taagtagaccgcctggggagtacggtcgcaagattaaaactcaaatgaattgacgggggcccgcacaagc

SEQ ID NO: 21 - DNA, 70 bp, synthetic construct
  misc_feature 1..70: note = synthetic
  source 1..70: mol_type = other DNA
taagtagaccgcctggggagtacggtcgcaagatcagaactcaaatgaattgacgggggcccgcacaagc

SEQ ID NO: 22 - DNA, 70 bp, synthetic construct
  misc_feature 1..70: note = synthetic
  source 1..70: mol_type = other DNA
gtcgtcagctcgtgttgtgaaatgttgggttaagtcccgcaacgagcgcaacccttatcctttgttgcca

SEQ ID NO: 23 - DNA, 70 bp, synthetic construct
  misc_feature 1..70: note = synthetic
  source 1..70: mol_type = other DNA
gtcgtcagctcgtgttgtgaaatgttgggttaagtcctgcaacgagcgcaacccttatcctttgttgcca

SEQ ID NO: 24 - DNA, 70 bp, synthetic construct
  misc_feature 1..70: note = synthetic
  source 1..70: mol_type = other DNA
gtcgtcagctcgtgtcgtgagatgttgggttaagtcccgtaacgagcgcaacccttgtccttagttacca

SEQ ID NO: 25 - DNA, 70 bp, synthetic construct
  misc_feature 1..70: note = synthetic
  source 1..70: mol_type = other DNA
gtcgtcagctcgtgttgtgaaatgttgggttaagtcccgcaacgagcgcaacccttatccttgtttgcca

SEQ ID NO: 26 - DNA, 70 bp, synthetic construct
  misc_feature 1..70: note = synthetic
  source 1..70: mol_type = other DNA
tcccgggccttgtacacaccgcccgtcacaccatgggagtgggttgcaaaagaagtaggtagcttaacct

SEQ ID NO: 27 - DNA, 70 bp, synthetic construct
  misc_feature 1..70: note = synthetic
  source 1..70: mol_type = other DNA
tcccgggccttgtacacaccgcccgtcacaccataggagtgggttgcaaaagaagtaggtagcttaacct

SEQ ID NO: 28 - DNA, 70 bp, synthetic construct
  misc_feature 1..70: note = synthetic
  source 1..70: mol_type = other DNA
tcccgggccttgtacacaccgcccgtcacaccatgggagtgggttgctccagaagtagctagtctaaccg

SEQ ID NO: 29 - DNA, 70 bp, synthetic construct
  misc_feature 1..70: note = synthetic
  source 1..70: mol_type = other DNA
tcccgggccttgtacacaccgcccgtcacaccatgggagtgggctgcaaaagaagcaggtagtttaacct

SEQ ID NO: 30 - DNA, 70 bp, synthetic construct
  misc_feature 1..70: note = synthetic
  source 1..70: mol_type = other DNA
tcgggagggcgcttaccactttgtgattcatgactggggtgaagtcgtaacaaggtaaccgtaggggaac

SEQ ID NO: 31 - DNA, 70 bp, synthetic construct
  misc_feature 1..70: note = synthetic
  source 1..70: mol_type = other DNA
tcgggagggcgcttaccactttgtgattcatgactgaggtgaagtcgtaacaaggtaaccgtaggggaac

SEQ ID NO: 32 - DNA, 70 bp, synthetic construct
  misc_feature 1..70: note = synthetic
  source 1..70: mol_type = other DNA
caagggggacggttaccacggagtgattcatgactagggtgaagtcgtaacaaggtagccgtaggggaac

SEQ ID NO: 33 - DNA, 70 bp, synthetic construct
  misc_feature 1..70: note = synthetic
  source 1..70: mol_type = other DNA
tcgggaggacgcttgccactttgtggttcatgactggggtgaagtcgtaacaaggtagcgctaggggaac

SEQ ID NO: 34 - DNA, 70 bp, synthetic construct
  misc_feature 1..70: note = synthetic
  source 1..70: mol_type = other DNA
tcgggaggacgcttgccactttgtggttcatgactgaggtgaagtcgtaacaaggtagcgctaggggaac

SEQ ID NO: 35 - DNA, 40 bp, synthetic construct
  misc_feature 1..40: note = synthetic
  source 1..40: mol_type = other DNA
atcgctagtaatcgtggatcagaatgccacggtgaatacg

SEQ ID NO: 36 - DNA, 40 bp, synthetic construct
  misc_feature 1..40: note = synthetic
  source 1..40: mol_type = other DNA
atcgctagtaatcgtgaatcagaatgtcacggtgaatacg

SEQ ID NO: 37 - DNA, 40 bp, synthetic construct
  misc_feature 1..40: note = synthetic
  source 1..40: mol_type = other DNA
atcgctagtaatcgcaaatcagaatgttgcggtgaatacg

SEQ ID NO: 38 - DNA, 18 bp, synthetic construct
  misc_feature 1..18: note = synthetic
  source 1..18: mol_type = other DNA
cggtggagcatgtggttt

SEQ ID NO: 39 - DNA, 20 bp, synthetic construct
  misc_feature 1..20: note = synthetic
  source 1..20: mol_type = other DNA
acgccttgcttttcactttc

SEQ ID NO: 40 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 41 - DNA, 1541 bp, synthetic construct
  misc_feature 1..1541: note = synthetic
  source 1..1541: mol_type = other DNA
aaattgaagagtttgatcatggctcagattgaacgctggcggcaggcctaacacatgcaagtcgaacggtaacaggaagaagcttgctctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 42 - DNA, 1541 bp, synthetic construct
  misc_feature 1..1541: note = synthetic
  source 1..1541: mol_type = other DNA
aaattgaagagtttgatcatggctcagattgaacgctggcggcaggcctaacacatgcaagtcgaacggtaacaggaagaagcttgctctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactagggtgaagtcgtaacaaggtaaccgtaggggaacctgcggttggatcacctcctta

SEQ ID NO: 43 - DNA, 1541 bp, synthetic construct
  misc_feature 1..1541: note = synthetic
  source 1..1541: mol_type = other DNA
aaattgaagagtttgatcatggctcagattgaacgctggcggcaggcctaacacatgcaagtcgaacggtaacaggaagaagcttgctctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactagggtgaagtcgtaacaaggtaaccgtaggggaacctgcggttggatcacctcctta

SEQ ID NO: 44 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtggctaataccgcataacgtcgcaagaccaaagagggggaccttcgggcctcttgccatcagatgtgcccagatgggattagctagttggtgaggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcggggaggaaggtgttgtggttaataaccgcagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtctacttggaggttgtgcccttgaggcgtggcttccggagctaacgcgttaagtagaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcgattaggtcgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactagggtgaagtcgtaacaaggtaaccgtaggggaacctgcggttggatcatgtggtta

SEQ ID NO: 45 - DNA, 1540 bp, synthetic construct
  misc_feature 1..1540: note = synthetic
  source 1..1540: mol_type = other DNA
aaattgaagagtttgatcatggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcgaggaggaaggcgttgtggttaataaccgcaacgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 46 - DNA, 1540 bp, synthetic construct
  misc_feature 1..1540: note = synthetic
  source 1..1540: mol_type = other DNA
aaattgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcgaggaggaaggcgttaaggttaataaccttggcgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 47 - DNA, 1540 bp, synthetic construct
  misc_feature 1..1540: note = synthetic
  source 1..1540: mol_type = other DNA
aaattgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgcgaagaaggccttcgggttgtaaagcactttcagcggggaggaaggcggtgaggttaataacctcatcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 48 - DNA, 1540 bp, synthetic construct
  misc_feature 1..1540: note = synthetic
  source 1..1540: mol_type = other DNA
aaattgaagagtttgatcatggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagcttgtaggtgaggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcggggaggaagggagtgaggttaataacctcattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctggagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 49 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagcagcttgctgcttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtacgaagaaggccttcgggttgtaaagtactttcagcggggaggaaggcgataaggttaataaccttgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 50 - DNA, 1542 bp, synthetic construct
  misc_feature 1..1542: note = synthetic
  source 1..1542: mol_type = other DNA
taattgaagagtttgatcatggctcagattgaacgctggcggcaggcttaacacatgcaagtcgagcggtagcacaggggagcttgctccctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgcgaagaaggccttcgggttgtaaagcactttcagcgaggaggaaggtggtgagcttaatacgctcatcaattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactagggtgaagtcgtaacaaggtaaccgtaggggaacctgcggttggatcatgtggtta

SEQ ID NO: 51 - DNA, 1540 bp, synthetic construct
  misc_feature 1..1540: note = synthetic
  source 1..1540: mol_type = other DNA
taattgaagagtttgatcatggctcagattgaacgctggcggcaggcctaacacatgcaagtcgagcggtaacaggggaagcttgcttctcgctgacgagcggcggacgggtgagtaatgtatggggatctgcccgatagagggggataactactggaaacggtggctaataccgcataatctcttaggagcaaagcaggggaccttcgggccttgcgctgtcggatgaacccatatgggattagctagtaggtaaggtaatggcttacctaggcgacgatccctagctggtctgagaggatgatcagccacactgggactgagacacggcccagactcctacgggaggcagcagtggggaatattgcacaatgggcgcaagcctgatgcagccatgccgcgtgtacgaagaaggccctagggttgtaaagtactttcagtcgggaggaaggcgttgatgttaataccatcaacgattgacgttaccgacagaagaagcaccggctaactccgtgccagcagccgcggtaatacggagggtgcaagcgttaatcggaattactgggcgtaaagcgcacgcaggcggttaattaagttagatgtgaaatccccgggcttaacctgggaatggcatctaagactggttagctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttggaggttgttccctagaggagtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgtatacaaagagaagcgacctcgcgagagcaagcggaactcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgatccatgactgaggtgaagtcgtaacaaggtaaccgtaggggaacctgcggttggatcatgtggtta

SEQ ID NO: 52 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtacgaagaaggccttagggttgtaaagtactttcagcggggaggaaggtgataaggttaatacccttattaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactgaggtgaagtcgtaacaaggtaaccgtaggggaacctgcggttggatcatgtggtta

SEQ ID NO: 53 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtacgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactgaggtgaagtcgtaacaaggtagcgctaggggaacctggcgctggatcatgtggtta

SEQ ID NO: 54 - DNA, 1533 bp, synthetic construct
  misc_feature 1..1533: note = synthetic
  source 1..1533: mol_type = other DNA
taattgaagagtttgatcatggctcagattgaacgctggcggcaggcctaacacatgcaagtcgaacggtaacatttctagcttgctagaagatgacgagtggcggacgggtgagtaatgcttgggaacttgcctttgcgagggggataacagttggaaacgactgctaataccgcataatgtcttcggaccaaacggggcttaggctccggcgcaaagagaggcccaagtgagattagctagttggtaaggtaacggcttaccaaggcgacgatctctagctgttctgagaggaagatcagccacactgggactgagacacggcccagactcctacgggaggcagcagtggggaatattgcacaatgggggaaaccctgatgcagccatgccgcgtgtgcgaagaaggccttcgggttgtaaagcactttcagttgtgaggaaaagttagtagttaatacctgctagccgtgacgttaacaacagaagaagcaccggctaactccgtgccagcagccgcggtaatacggagggtgcgagcgttaatcggaattactgggcgtaaagcgcacgcaggcggtttgttaagctagatgtgaaagccccgggctcaacctgggatggtcatttagaactggcagactagagtcttggagaggggagtggaattccaggtgtagcggtgaaatgcgtagatatctggaggaacatcagtggcgaaggcgactccctggccaaagactgacgctcatgtgcgaaagtgtgggtagcgaacaggattagataccctggtagtccacaccgtaaacgctgtctactagctgtgtgtgtctttaagacgtgcgtagcgaagctaacgcgctaagtagaccgcctggggagtacggccgcaaggttaaaactcaaatgaattgacgggggcccgcacaagcggtggagcatgtggtttaattcgatgcaacgcgaagaaccttacctacacttgacatgctgagaagttactagagatagtttcgtgccttcgggaactcagacacaggtgctgcatggctgtcgtcagctcgtgtcgtgagatgttgggttaagtcccgcaacgagcgcaacccttgtccttagttgccagccttaagttgggcactctaaggagactgccggtgacaaaccggaggaaggtggggacgacgtcaagtcatcatggcccttacgtgtagggctacacacgtgctacaatggcatttacagagggaagcgagacagtgatgtggagcggaccccttaaagaatgtcgtagtccggattggagtctgcaactcgactccatgaagtcggaatcgctagtaatcgcaggtcagaatactgcggtgaatacgttcccgggccttgtacacaccgcccgtcacaccatgggagtgggatgcaaaagaagtagttagtctaaccttcgggaggacgattaccactttgtgtttcatgactgaggtgaagtcgtaacaaggtaaccctaggggaacctggggttggatcatgtggtta

SEQ ID NO: 55 - DNA, 1535 bp, synthetic construct
  misc_feature 1..1535: note = synthetic
  source 1..1535: mol_type = other DNA
aaactgaagagtttgatcatggctcagattgaacgctggcggcaggcctaacacatgcaagtcgagcggtaacaggagaagcttgcttcttgctgacgagcggcggacgggtgagtaatacttgggtatctgcccgatagtgggggacaacacggggaaactcgtgctaataccgcatacgtcctacgggagaaagcaggcttaggcttgcgctatcggatgagcccaagtcggattagctagttggtgaggtaaaggctcaccaaggcgacgatccgtaactggtctgagaggatgatcagtcacaccgggactgagacacggcccggactcctacgggaggcagcagtggggaatattggacaatgggggcaaccctgatccagccatgccgcgtgtgcgaagaaggccttagggttgtaaagcactttcagtgaggaggaaaagttagtggttaatacccactagccgtgacgttactcacagaagaagcaccggcaaactccgtgccagcagccgcggtaatacggagggtgcgagcgttaatcggaattactgggcgtaaagcgcgcgtaggcggtttgttaagccggttgtgaaagccctaggctcaacctaggaactgcacccggaactggcaagctagagtacagtagaggaaggtggaattccacgtgtagcggtgaaatgcgtagatatgtggaggaacatcagtggcgaaggcggccttctggactgatactgacgctgaggtgcgaaagcgtggggagcaaacaggattagataccctggtagtccacgccgtaaacgatgtcaactagttgttggaacccttgaggttttagtaacgcagctaacgcattaagttgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgtaacgagcgcaacccctatccttatttgccagcgagttaagtcgggaactctaaggagactgccggtgacaaaccggaggaaggtggggatgacgtcaagtcatcatggcccttacgagtagggctacacacgtgctacaatggttggtacagcaggttgctaacccgcgagggggcgctaatccgtcaaaaccgatcgtagtccggatcgcagtctgcaactcgactgcgtgaagtcggaatcgctagtaatcgcggatcagaatgccgcggtgaatacgttcccgggccttgtacacaccgcccgtcacaccatgggagttgattgcaccagaagtagctagcttaaccttcgggagggcggttaccacggtgtggttaatgactgaggtgaagtcgtaacaaggtagccgtaggggaacctgcggctggatcatgtggtta

SEQ ID NO: 56 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgcgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactagggtgaagtcgtaacaaggtagccgtaggggaacctgcggctggatcatgtggtta

SEQ ID NO: 57 - DNA, 1538 bp, synthetic construct
  misc_feature 1..1538: note = synthetic
  source 1..1538: mol_type = other DNA
taactgaagagtttgatcatggctcagattgaacgctggcggcaggcttaacacatgcaagtcgagcgggggaaggtagcttgctaccggacctagcggcggacgggtgagtaatgcttaggaatctgcctattagtgggggacaacatctcgaaagggatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcgcaagcctgatccagccatgccgcgtgtgcgaagaaggccttatggttgtaaagcactttaagcgaggaggaggctactttagataatacctagagatagtggacgttactcgcagaataagcaccggctaactctgtgccagcagccgcggtaatacagagggtgcaagcgttaatcggatttactgggcgtaaagcgcgcgtaggcggctaattaagtcaaatgtgaaatccccgagcttaacttgggaattgcattcgatactggttagctagagtgtgggagaggatggtagaattccaggtgtagcggtgaaatgcgtagagatctggaggaataccgatggcgaaggcagccatctggcctaacactgacgctgaggtgcgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacacagcgatgtgatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtagctagcctaactgcaaagagggcggttaccacggtgtggccgatgactgaggtgaagtcgtaacaaggtagccgtaggggaacctgcggctggatcatgtggtat

SEQ ID NO: 58 - DNA, 1537 bp, synthetic construct
  misc_feature 1..1537: note = synthetic
  source 1..1537: mol_type = other DNA
aaactgaagagtttgatcctggctcagattgaacgctagcgggatgctttacacatgcaagtcgaacggcagcgcgagagagcttgctctcttggcggcgagtggcggacgggtgagtaatatatcggaacgtgcccagtagcgggggataactactcgaaagagtggctaataccgcatacgccctacgggggaaagggggggatcgcaagacctctcactattggagcggccgatatcggattagctagttggtggggtaaaggctcaccaaggcaacgatccgtagctggtttgagaggacgaccagccacactgggactgagacacggcccagactcctacgggaggcagcagtggggaattttggacaatgggggaaaccctgatccagccatcccgcgtgtacgatgaaggccttcgggttgtaaagtacttttggcagagaagaaaaggtacctcctaatacgaggtactgctgacggtatctgcagaataagcaccggctaactacgtgccagcagccgcggtaatacgtagggtgcaagcgttaatcggaattactgggcgtaaagcgtgtgtaggcggttcggaaagaaagatgtgaaatcccagggctcaaccttggaactgcatttttaactgccgagctagagtatgtcagaggggggtagaattccacgtgtagcagtgaaatgcgtagatatgtggaggaataccgatggcgaaggcagccccctgggataatactgacgctcagacacgaaagcgtggggagcaaacaggattagataccctggtagtccacgccctaaacgatgtcaactagctgttggggccgttaggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaaagccgaagagatttggccgtgctcgcaagagaaccggaacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatgggtagggcttcacacgtcatacaatggtcgggacagagggtcgccaacccgcgagggggagccaatctcagaaacccgatcgtagtccggatcgcagtctgcaactcgactgcgtgaagtcggaatcgctagtaatcgcggatcagaatgtcgcggtgaatacgttcccgggtcttgtacacaccgcccgtcacaccatgggagtgggtttcaccagaagtaggtagcctaaccgtaaggagggcgcttaccacggtgggattcatgactgaggtgaagtcgtaacaaggtagccgtatcggaaggtgcggctggatcatgtggttt

SEQ ID NO: 59 - DNA, 1531 bp, synthetic construct
  misc_feature 1..1531: note = synthetic
  source 1..1531: mol_type = other DNA
gaactgaagagtttgatcctggctcagattgaacgctggcgggatgctttacacatgcaagtcggacggcagcacgggcttcggcctggtggcgagtggcgaacgggtgagtaatgtatcggaacgtgcccagtagcgggggataactacgcgaaagcgtggctaataccgcatacgccctacgggggaaagcgggggaccttcgggcctcgcactattggagcggccgatatcggattagctagttggtggggtaacggcctaccaaggcgacgatccgtagctggtttgagaggacgaccagccacactgggactgagacacggcccagactcctacgggaggcagcagtggggaattttggacaatgggggcaaccctgatccagccatcccgcgtgtgcgatgaaggccttcgggttgtaaagcacttttggcaggaaagaaacggcacgggctaatatcctgtgcaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagggcttaaccttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagtgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatcccgaagagatttgggagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggttgccaacccgcgagggggagccaatcccagaaacccggtcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgcaaggggggcgattaccacggtaggattcatgactgaggtgaagtcgtaacaaggtagccgtatcggaaggtgcggctggatcatgtggttt

SEQ ID NO: 60 - DNA, 1533 bp, synthetic construct
  misc_feature 1..1533: note = synthetic
  source 1..1533: mol_type = other DNA
gaactgaagagtttgatcctggctcagattgaacgctggcggcatgccttacacatgcaagtcgaacggcagcacgggtgcttgcacctggtggcgagtggcgaacgggtgagtaatacatcggaacatgtcctgtagtgggggatagcccggcgaaagccggattaataccgcatacgatccatggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgaaagcctgatccagcaatgccgcgtgtgcgaagaaggccttcgggttgtaaagcacttttgtccggaaagaaatccttggctctaatacagtcgggggatgacggtaccggaagaataagcaccggctaactacgtgccagcagccgcggtaatacgtagggtgcgagcgttaatcggaattactgggcgtaaagcgtgcgcaggcggtttgctaagaccgatgtgaaatccccgggctcaacctgggaactgcattggtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatggtcggaatcctgctgagaggtgggagtgctcgaaagagaaccggcgcacaggtgctgcatggctgtcgtcagctcgtgtcgtgagatgttgggttaagtcccgcaacgagcgcaacccttgtccttagttgctacgcaagagcactctaaggagactgccggtgacaaaccggaggaaggtggggatgacgtcaagtcctcatggcccttatgggtagggcttcacacgtcatacaatggtcggaacagagggttgccaacccgcgagggggagctaatcccagaaaaccgatcgtagtccggattgcactctgcaactcgagtgcatgaagctggaatcgctagtaatcgcggatcagcatgccgcggtgaatacgttcccgggtcttgtacacaccgcccgtcacaccatgggagtgggttttaccagaagtggctagtctaaccgcaaggaggacggtcaccacggtaggattcatgactgaggtgaagtcgtaacaaggtagccgtatcggaaggtgcggctggatcatgtggttt

SEQ ID NO: 61 - DNA, 1541 bp, synthetic construct
  misc_feature 1..1541: note = synthetic
  source 1..1541: mol_type = other DNA
gaacataagagtttgatcctggctcagattgaacgctggcggcatgctttacacatgcaagtcggacggcagcacagggaagcttgcttctcgggtggcgagtggcgaacgggtgagtaacatatcggaacgtaccgggtagcgggggataactgatcgaaagatcagctaataccgcatacgtcttgagagggaaagcaggggaccttcgggccttgcgctatccgagcggccgatatctgattagctggttggcggggtaaaggcccaccaaggcgacgatcagtagcgggtctgagaggatgatccgccacactgggactgagacacggcccagactcctacgggaggcagcagtggggaattttggacaatgggcgcaagcctgatccagccatgccgcgtgtccgaagaaggccttcgggttgtaaaggacttttgtcagggaagaaaaggccgttgccaatatcggcggccgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacccgggaactgcgttctgaactgggtgactcgagtgtgtcagagggaggtggaattccacgtgtagcagtgaaatgcgtagagatgtggaggaataccgatggcgaaggcagcctcctgggataacactgacgttcatgtccgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggttttgacatgtgcggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcggttgggcactctaatgagactgccggtgacaagccggaggaaggtggggatgacgtcaagtcctcatggcccttatgaccagggcttcacacgtcatacaatggtcggtacagagggtagccaagccgcgaggcggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagtccgcttaccacggtatgcttcatgactcgggtgaagtcgtaacaaggtagccgtaggggaacctgcggctggatcatgtggttt

SEQ ID NO: 62 - DNA, 1531 bp, synthetic construct
  misc_feature 1..1531: note = synthetic
  source 1..1531: mol_type = other DNA
ggagagtttgatcctggctcagaacgaacgctggcggcgtgcctaacacatgcaagtcgaacggactttaagacttcggttttaaagttagtggcgcacgggtgagtaacgcgtggatatctgcctatcagtgggggacaactacaggaaactgtagctaataccgcatacgctgtacgcaggaaagcgggggatcttcggacctcgcgctgatagatgagtccgcgtctgattagctagttggtggggtaaaggcctaccaaggcgatgatcagtaactggtttgagaggatgatcagtcacactggaactgagacacggtccagactcctacgggaggcagcagtggggaatattgcacaatgggggaaaccctgatgcagcgacgccgcgtgcgcgaagaaggccttcgggttgtaaaccgctttcaattgggaagaagagatgagtactaatactgctctgatttgacggtacctttagaagaagcaccggctaatttcgtgccagcagccgcggtaatacgaaaggtgcaagcgttgttcggatttactgggcgtaaagagatcgtaggtggtttgttaagtcggatgtgaaatcccagggctcaaccctggaactgcattcgatactggcagactagagtttgggaggggtaagcggaattccgtgtgtagcagtgaaatgcgtagatatacggaggaacacctgaggcgaaggcggcttactggaccaatactgacactgaggatcgaaagcgtgggtagcaaacaggattagataccctggtagtccacgccctaaacgatgtcaactagccgtagcgggtatcgacccctgctgtggcgaagctaacgcgataagttgaccgcctggggagtacggtcgcaagattaaaactcaaaggaattgacgggggcccgcacaagcggtggagcatgtggtttaattcgacgcaacgcgaagaaccttacctgggtttgacatcctaagaatactttagagatagagtagtgccttcgggagcttagagacaggtgctgcatggctgtcgtcagctcgtgtcgtgagatgttgggttaagtcccgcaacgagcgcaacccctatcgttagttgccatcatttagttgggcactctagcgagactgccggtgacaaaccggaggaaggtggggacgacgtcaagtcatcatggcccttatgtccagggctacacacgtgctacaatggcgactacaacaagttgcgaacccgcgagggggagccaatcttataaaagtcgtctcagttcggattggagtctgcaactcgactccatgaagttggaatcgctagtaatcgcggatcagcatgccgcggtgaatacgttcccgggccttgtacacaccgcccgtcacaccatgggaattggttgcaccagaagccgccgggctaaccttcgggaggcaggcgtctacggtgtggtcggtaactgaggtgaagtcgtaacaaggtatccctaccggaaggtggggatggatcatgtggttt

SEQ ID NO: 63 - DNA, 1484 bp, synthetic construct
  misc_feature 1..1484: note = synthetic
  source 1..1484: mol_type = other DNA
caacctgagagtttgatcctggctcagagcgaacgctggcggcaggcctaacacatgcaagtcgaacggatccttcgggattagtggcggacgggtgagtaacacgtgggaacgtgccctttggttcggaacaactcagggaaacttgagctaataccggatgtgcccttcgggggaaagatttatcgccattggagcggcccgcgtctgattagctagttggtgaggtaaaggctcaccaaggcgacgatcagtagctggtctgagaggatgatcagccacattgggactgagacacggcccaaactcctacgggaggcagcagtggggaatcttgcgcaatgggcgaaagcctgacgcagccatgccgcgtgaacgatgaaggtcttaggattgtaaaattctttcaccggggacgataatgacggtacccggagaagaagccccggctaacttcgtgccagcagccgcggtaatacgaagggggctagcgttgctcggaattactgggcgtaaagggagcgtaggcggactgtttagtcagaggtgaaagcccagggctcaaccttggaattgcctttgatactggcagtcttgagtacggaagaggtatgtggaactccgagtgtagaggtgaaattcgtagatattcggaagaacaccagtggcgaaggcgacatactggtccgttactgacgctgaggctcgaaagcgtggggagcaaacaggattagataccctggtagtccacgccgtaaacgatgagtgctagttgtcggcatgcatgcatgtcggtgacgcagctaacgcattaagcactccgcctggggagtacggtcgcaagattaaaactcaaaggaattgacgggggcccgcacaagcggtggagcatgtggtttaattcgaagcaacgcgcagaaccttaccaccttttgacatgcctggaccgccacagagatgtggttttcccttcggggactgggacacaggtgctgcatggctgtcgtcagctcgtgtcgtgagatgttgggttaagtcccgcaacgagcgcaaccctcgtgattagttgccatcaggtttggctgggcactctaatcatactgccggagttaatccggaggaaggcggggatgacgtcaagtcctcatggcccttacaaggtgggctacacacgtgctacaatggcgactacagagggctgcaatcccgcgagggggagccaatccctaaaagtcgtctcagttcggattgttctctgcaactcgagagcatgaagttggaatcgctagtaatcgcggatcagcatgccgcggtgaatacgttcccgggccttgtacacaccgcccgtcacaccatgggagttggctttacccgaaggcgctgcgctaaccgcaagggggcaggcgaccacggtagggtcagcgactgaggtgaagtcgtaacaaggtagccgtaggggaacctgcggctggatcatgtggttt

SEQ ID NO: 64 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtacttttagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggtta

SEQ ID NO: 65 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggctaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggtta

SEQ ID NO: 66 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttgaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggtta

SEQ ID NO: 67 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcctgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggtta

SEQ ID NO: 68 - DNA, 1542 bp, synthetic construct
  misc_feature 1..1542: note = synthetic
  source 1..1542: mol_type = other DNA
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccataggagtgggttgcaaaagaagtaggtagcttaaccttcgggagggcgcttaccactttgtgattcatgactggggtgaagtcgtaacaaggtaaccgtaggggaacctgcggttggatcatgtggtta

SEQ ID NO: 69 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgcgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 70 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcacttttagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 71 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggctaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 72 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttgaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 73 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcctgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 74 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccataggagtgggttgctccagaagtagctagtctaaccgcaagggggacggttaccacggagtgattcatgactggggtgaagtcgtaacaaggtagccgtaggggaacctgcggctggatcatgtggtta

SEQ ID NO: 75 - DNA, 1536 bp, synthetic construct
  misc_feature 1..1536: note = synthetic
  source 1..1536: mol_type = other DNA
gaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactgaggtgaagtcgtaacaaggtagccgtaggggaacctgcggctggatcatgtggtta

SEQ ID NO: 76 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtacgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 77 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtacttttagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 78 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagatcaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 79 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattagaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 80 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcctgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 81 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccataggagtgggctgcaaaagaagcaggtagtttaaccttcgggaggacgcttgccactttgtggttcatgactggggtgaagtcgtaacaaggtagcgctaggggaacctggcgctggatcatgtggtta

SEQ ID NO: 82 - DNA, 1543 bp, synthetic construct
  misc_feature 1..1543: note = synthetic
  source 1..1543: mol_type = other DNA
taattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactgaggtgaagtcgtaacaaggtagcgctaggggaacctggcgctggatcatgtggtta

SEQ ID NO: 83 - protein, 6 aa, synthetic construct
  REGION 1..6: note = synthetic
GGSGGS

SEQ ID NO: 84 - protein, 522 aa, synthetic construct
  REGION 1..522: note = synthetic
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGGSGGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGGSGGSIKIATRKYLGKQNVYDIGVERDHNFALKNGFIASNCFN

SEQ ID NO: 85 - DNA, 10811 bp, synthetic construct
  misc_feature 1..10811: note = synthetic
  source 1..10811: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaggatactctctaatcctgacctgttggagtttgcttccggtctggttcgctttgaagctcgaattaaaacgagatatttgaagtctttcgggcttcctcttaatctttttgatgcaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatctctcacctaccaaacaatgcccccctgcaaaaaataaattcatataaaaaacatacagataaccatctgcggtgataaattatctctggcggtgttgacataaataccactggcggttatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacttgtataccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctccagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 86 - DNA, 10964 bp, synthetic construct
  misc_feature 1..10964: note = synthetic
  source 1..10964: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattgcaggggcataatgtttttggtacaaccgatttaggtttatgttctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaggatactctctaatcctgacctgttggagtttgcttccggtctggttcgctttgaagctcgaattaaaacgagatacttgaagtctttcgggcttcctcttaatctttttgatgcaatccgctttgcttctgactataatagtcagggtaaagacctgatttttgatttatggtcattctcgttttctgaactgtttaaagcatttgaagggaattcaatgaatatttatgacgattccgcagtattggccgctatccagtctaaacattttactattaccccctctggcaaaacttcttttgcaaaagcctctcgctattttggtttctatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatctctcacctaccaaacaatgcccctgcaaaaaataaattcatataaaaaacatacagataaccatctgcggtgataaattatctctggcggtgttgacataaatacactggcggttatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttgaactgaagagtttgatcatggctcagattgaacgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtaggggaacctgcggctggatcacttgtataccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggtcaagtgaagaagcgcatacggtggatgccttggcagtcagaggcgatgaaagacgtggtagcctgcgaaaagcttcggggagtcggcaaacagactttgatccggagatctctgaatgggggaacccacctaggataacctaggtatcttgtactgaatccataggtgcaagaggcgaaccaggggaactgaaacatctaagtaccctgaggaaaagaaatcaaccgagattcccttagtagtggcgagcgaacggggattagcccttaagcttcattgattttagcggaacgctctggaaagtgcggccatagtgggtgatagccccgtacgcgaaaggatctttgaagtgaaatcgagtaggacggagcacgagaaactttgtctgaacatggggggaccatcctccaaggctaaatactactgactgaccgatagtgaaccagtaccgtgagggaaaggcgaaaagaaccccggagaggggagtgaaatagaacctgaaaccgtatgcgtacaagcagtgggagcctacttgttaggtgactgcgtaccttttgtataatgggtcagcgacttatattcagtggcaagcttaaccgtatagggtaggcgtagcgaaagcgagtcttaatagggcgtttagtcgctgggtatagacccgaaaccgggcgatctatccatgagcaggttgaaggttaggtaacactgactggaggaccgaacccactcccgttgaaaaggtaggggatgacttgtggatcggagtgaaaggctaatcaagctcggagatagctggttctcctcgaaagctatttaggtagcgcctcatgtatcactctggggggtagagcactgtttcggctagggggtcatcccgacttaccaaaccgatgcaaactccgaatacccagaagtgccgagcatgggagacacacggcgggtgctaacgtccgtcgtgaaaagggaaacaacccagaccgccagctaaggtcccaaagttgtggttaagtggtaaacgatgtgggaaggcttagacagctaggaggttggcttagaagcagccaccctttaaagaaagcgtaatagctcactagtcgagtcggcctgcgcggaagatgtaacggggctcaaaccacacaccgaagctgcgggtgtcacgtaagtgacgcggtagaggagcgttctgtaagcctgtgaaggtgagttgagaagcttgctggaggtatcagaagtgcgaatgctgacatgagtaacgacaatgggtgtgaaaaacacccacgccgaaagaccaagggttcctgcgcaacgttaatcgacgcagggttagtcggttcctaaggcgaggctgaaaagcgtagtcgatgggaaacaggttaatattcctgtacttctggttactgcgatggagggacggagaaggctaggccagcttggcgttggttgtccaagtttaaggtggtaggctgaaatcttaggtaaatccggggtttcaaggccgagagctgatgacgagtcgtcttttagatgacgaagtggttgatgccatgcttccaagaaaagcttctaagcttcaggtaaccaggaaccgtaccccaaaccgacacaggtggtcgggtagagaataccaaggcgcttgagagaactcgggtgaaggaactaggcaaaatggcaccgtaacttcgggagaaggtgcgccggctagggtgaaggatttactccgtaagctctggctggtcgaagataccaggccgctgcgactgtttattaaaaacacagcactctgcaaacacgaaagtggacgtatagggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaacgatggcggcgctgtctccacccgagactcagtgaaattgaaatcgctgtgaagatgcagtgtatccgcggctagacggaaagaccccgtgaacctttactgtagctttgcactggactttgagcctgcttgtgtaggataggtgggaggctttgaagcgtggacgccagttcgcgtggagccatccttgaaataccaccctggcatgcttgaggttctaactctggtccgtaatccggatcgaggacagtgtatggtgggcagtttgactggggcggtctcctcctaaagagtaacggaggagtacgaaggtgcgctcagaccggtcggaaatcggtcgcagagtataaaggcaaaagcgcgcttgactgcgagacagacacgtcgagcaggtacgaaagtaggtcttagtgatccggtggttctgtatggaagggccatcgctcaacggataaaaggtactccggggataacaggctgataccgcccaagagttcatatcgacggcggtgtttggcacctcgatgtcggctcatcacatcctggggctgaagccggtcccaagggtatggctgttcgccatttaaagtggtacgcgagctgggtttagaacgtcgtgagacagttcggtccctatctgccgtggacgtttgagatttgagaggggctgctcctagtacgagaggaccggagtggacgaacctctggtgttccggttgtcacgccagtggcattgccgggtagctatgttcggaaaagataaccgctgaaagcatctaagcgggaaacttgcctcaagatgagatctcactgggaacttgattcccctgaagggccgtcgaagactacgacgttgataggctgggtgtgtaagcgttgtgaggcgttgagctaaccagtactaattgcccgtgaggcttgaccatacaacgccgaagctgttttggcggatgagagaagattttcagcctgatacagattaaatcagaacgcagaagcggtctgataaaacagaattttgcttgacgatcatagagcgttggaaccacctgatcccttcccgaactcagaagtgaaacgacgcatcgccgatggtagtgtggggtctccccatgtgagagtaggtcatcgtcaagctccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttttacatgctcgtaaattaggatgggatattatttttcttgttcaggacttatccattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccccactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattctgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccggtttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 87 - DNA, 10977 bp, synthetic construct
  misc_feature 1..10977: note = synthetic
  source 1..10977: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaggatactctctaatcctgacctgttggagtttgcttccggtctggttcgctttgaagctcgaattaaaacgagatatttgaagtctttcgggcttcctcttaatctttttgatgcaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatctctcacctaccaaacaatgcccccctgcaaaaaataaattcatataaaaaacatacagataaccatctgcggtgataaattatctctggcggtgttgacataaataccactggcggttatactgagcacgggtaccggccgctgagaaaaagcgaagcggcactgctcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaactttttaattgaagagtttgatcatggctcagattgaacgctggcggcaggcctaacacatgcaagtcgagcggcagcacagaggaacttgttccttgggtggcgagcggcggacgggtgagtaatgcctgggaaattgcccggtagagggggataaccattggaaacgatggctaataccgcataacctcgcaagagcaaagcaggggaccttcgggccttgcgctaccggatatgcccaggtgggattagctagttggtgaggtaagggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtatgaagaaggccttcgggttgtaaagtactttcagtagggaggaaggtggttaagttaataccttaatcatttgacgttacctacagaagaagcaccggctaactccgtgccagcagccgcggtaatacggagggtgcaagcgttaatcggaattactgggcgtaaagcgcatgcaggtggtttgttaagtcagatgtgaaagccctgggctcaacctaggaatcgcatttgaaactgacaagctagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtgacctagagtcgtggctttcggagctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaatctagcggagacgctggagtgccttcgggagctctgagacaggtgctgcatggctgtcgtcagctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgtatacagagggcagcgataccgcgaggtggagcgaatctcacaaagtacgtcgtagtccggattggagtctgcaactcgactccatgaagtcggaatcgctagtaatcgcaaatcagaatgttgcggtgaatacgttcccgggccttgtacacaccgcccgtcacaccatgggagtgggctgcaaaagaagcaggtagtttaaccttcgggaggacgcttgccactttgtggttcatgactggggtgaagtcgtaacaaggtagcgctaggggaacctggcgctggatcacttgtataccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagtgactaagcgtacacggtggatgcctgggcagtcagaggcgatgaaggacgtactaacttgcgataagcgcagataaggcagtaagagccgtttgagtctgcgatttccgaatggggaaacccaactgcataagcagttactgttaactgaatacataggttaacagagcaaaccgggggaactgaaacatctaagtaccccgaggagaagaaatcaaccgagattccggtagtagcggcgagcgaacctggattagcccttaagcactcggtgaagtaggtgaacaagctggaaagcttggcgatacagggtgatagccccgtaaccgacgcttcatcgagcgtgaaatcgagtagggcgggacacgtgatatcctgtctgaatatggggggaccatcctccaaggctaaatactcctgactgaccgatagtgaaccagtaccgtgagggaaaggcgaaaagaacccctgtgaggggagtgaaatagaacctgaaaccgtgtacgtacaagcagtaggagcaccttcgtggtgtgactgcgtaccttttgtataatgggtcagcgacttatattcagtggcaaggttaaccgtataggggagccgtagcgaaagcgagtcttaattgggcgctcagtctctggatatagacccgaaaccgggtgatctagccatgggcaggttgaaggttgagtaacatcaactggaggaccgaaccgactaatgttgaaaaattagcggatgacttgtggctaggggtgaaaggccaatcaaactcggagatagctggttctccccgaaagctatttaggtagcgcctcggacgaatactactgggggtagagcactgttaaggctagggggtcatcccgacttaccaaccctttgcaaactccgaataccagtaagtactatccgggagacacacggcgggtgctaacgtccgtcgtggagagggaaacaacccagaccgccagctaaggtcccaaagtattgctaagtgggaaacgatgtgggaaggctcagacagctaggatgttggcttagaagcagccatcatttaaagaaagcgtaatagctcactagtcgagtcggcctgcgcggaagatgtaacggggctaagcaatacaccgaagctgcggcaatgtcttttagatattgggtaggggagcgttctgtaagccgttgaaggtgaatcgtaaggtttgctggaggtatcagaagtgcgaatgctgacatgagtaacgacaaagggggtgaaaaacctcctcgccggaagaccaagggttcctgtccaacgttaatcggggcagggtgagtcgacccctaaggtgaggccgaaaggcgtaatcgatgggaaacgggttaatattcccgtacttctgactattgcgatggggggacggagaaggctaggtgggccaggcgacggttgtcctggttcaagtgcgtaggcttgagagttaggtaaatccggctctctttaaggctgagacacgacgtcgagctgctacggcagtgaagtcattgatgccatgcttccaggaaaagcctctaagcttcagatagtcaggaatcgtaccccaaaccgacacaggtggtcgggtagagaataccaaggcgcttgagagaactcgggtgaaggaactaggcaaaatggtaccgtaacttcgggagaaggtacgctcttgatggtgaagtccctcgcggatggagctgacgagagtcgcagataccaggtggctgcaactgtttattaaaaacacagcactgtgcaaaatcgcaagatgacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccacgctgtctccacccgagactcagtgaaattgaaatcgctgtgaagatgcagtgtacccgcggctagacggaaagaccccgtgaacctttactacagcttggcactgaacattgaacctacatgtgtaggataggtgggagtctatgaagacgtgacgccagttgcgttggagccgtccttgaaataccacccttgtatgtttgatgttctaacgttggcccctaatcggggttgcggacagtgcctggtgggtagtttgactggggcggtctcctcccaaagagtaacggaggagcacgaaggtgggctaatcacggttggacatcgtgaggttagtgcaatggcataagcccgcttaactgcgagaatgacggttcgagcaggtgcgaaagcaggtcatagtgatccggtggttctgtatggaagggccatcgctcaacggataaaaggtactccggggataacaggctgataccgcccaagagttcatatcgacggcggtgtttggcacctcgatgtcggctcatcacatcctggggctgaagtcggtcccaagggtatggctgttcgccatttaaagtggtacgcgagctgggtttagaacgtcgtgagacagttcggtccctatctgccgtgggcgttggaagattgaagggggctgctcctagtacgagaggaccggagtggacgaacctctggtgttcgggttgtgtcgccagacgcattgcccggtagctaagttcggaattgataagcgctgaaagcatctaagcgcgaagcgagccctgagatgagtcttccctgacggtttaaccgtcctaaagggttgttcgagactagaacgttgataggcagggtgtgtaagcgttgtgaggcgttgagctaacctgtactaattgcccgtgaggcttaaccatacaacgccgaagctgttttggcggatgagagaagattttcagcctgatacagattaaatcagaacgcagaagcggtctgataaaacagaattttgcttggcgaccatagcgttttggacccacctgactccatcccgaactcagaagtgaaacgaaacagcgtcgatggtagtgtggggtctccccatgtgagagtagaacatcgccaggcttcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 88 - DNA, 5855 bp, synthetic construct
  misc_feature 1..5855: note = synthetic
  source 1..5855: mol_type = other DNA
aacaccccttgtattactgtttatgtaagcagacagttttattgttcatgatgatatatttttatcttgtgcaatgtaacatcagagattttgagacacaacgtggctttccatcaaaaaaatattgacaacataaaaaactttgtgttatacttgtggaattgtgagcggataacaattctatatctgttattttttccaaccacagatctatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaagtcgaccggctgctaacaaagcccgcggccgctgaagatcgatctcgacgagtgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtcaccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacagagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcggatctgtatggtgcactctcagtacaatctgctctgatgccgcatagttaagccagtatacactccgctatcgctacgtgactgcctcgacctgcagcaattccaacgccatcaaaaataattcgcgtctggccttcctgtagccagctttcatcaacattaaatgtgagcgagtaacaacccgtcggattctccgtgggaacaaacggcggattgaccgtaatgggataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatccgtaatcatggtcatagctgtttcctgtgtgaaattgttatccgctcacaattccacacaacatacgagccggaagcataaagtgtaaagcctggggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattccatggtgtcggaattgctgcaggtcgagggggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 89 - DNA, 5855 bp, synthetic construct
  misc_feature 1..5855: note = synthetic
  source 1..5855: mol_type = other DNA
ttgagacacaacgtggctttccatcaaaaaaatattgacaacataaaaaactttgtgttatacttgtggaattgtgagcggataacaattctatatctgttattttttcatatacaagatctatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaagtcgaccggctgctaacaaagcccgcggccgctgaagatcgatctcgacgagtgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtcaccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacagagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcggatctgtatggtgcactctcagtacaatctgctctgatgccgcatagttaagccagtatacactccgctatcgctacgtgactgcctcgacctgcagcaattccaacgccatcaaaaataattcgcgtctggccttcctgtagccagctttcatcaacattaaatgtgagcgagtaacaacccgtcggattctccgtgggaacaaacggcggattgaccgtaatgggataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaatccgtaatcatggtcatagctgtttcctgtgtgaaattgttatccgctcacaattccacacaacatacgagccggaagcataaagtgtaaagcctggggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattccatggtgtcggaattgctgcaggtcgagggggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagatt

SEQ ID NO: 90 - DNA, 4603 bp, synthetic construct
  misc_feature 1..4603: note = synthetic
  source 1..4603: mol_type = other DNA
tcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaagaggacatccggtggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcgctgaggattctagagcacagctaacaccacgtcgtccctatctgctgccctaggtctatgagtggttgctggataactttacgggcatgcataaggctcgtatgatatattcagggagaccacaacggtttccctctacaaataattttgtttaactttatatctgttattttttcaaccacagatctatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaaacttaattaacggcactcctcagccaagtcaaaagcctccgaccggaggcttttgactacatgcccatggcgtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgaaggccaaataggccgt

SEQ ID NO: 91 - DNA, 4604 bp, synthetic construct
  misc_feature 1..4604: note = synthetic
  source 1..4604: mol_type = other DNA
tcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaagaggacatccggtggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcgctgaggattctagagcacagctaacaccacgtcgtccctatctgctgccctaggtctatgagtggttgctggataactttacgggcatgcataaggctcgtatgatatattcagggagaccacaacggtttccctctacaaataattttgtttaactttatatctgttattttttcatatacaagatctatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaaacttaattaacggcactcctcagccaagtcaaaagcctccgaccggaggcttttgactacatgcccatggcgtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgaaggccaaataggccgt

SEQ ID NO: 92 - DNA, 4291 bp, synthetic construct
  misc_feature 1..4291: note = synthetic
  source 1..4291: mol_type = other DNA
tcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaagaggacatccggtttgttcagaacgctcggtcttgcacaccgggcgttttttctttgtgagtccatagtacacttgaataaataaaaacagccgttgccagaaagaggcacggctgtttttattttagacttagggaccctcacagctaacaccacgtcgtccctatctgctgccctaggtctatgagtggttgctggataactttacgggcatgcataaggctcgtaatatatattcagggagaccacaacggtttccctctacaaataattttgtttaactttatatctgttattttttcatatacaagatctatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaaacttaattaacggcactcctcagccaagtcaaaagcctccgaccggaggcttttgactacatgcccatggcgtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgaaggccaaataggccgt

SEQ ID NO: 93 - DNA, 4729 bp, synthetic construct
  misc_feature 1..4729: note = synthetic
  source 1..4729: mol_type = other DNA
tcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaagaggacatccggtttgttcagaacgctcggtcttgcacaccgggcgttttttctttgtgagtccatagtacacttgaataaataaaaacagccgttgccagaaagaggcacggctgtttttattttagacttagggaccctcacagctaacaccacgtcgtccctatctgctgccctaggtctatgagtggttgctggataactttacgggcatgcataaggctcgtaatatatattcagggagaccacaacggtttccctctacaaataattttgtttaactttatatctgttattttttcatatacaagatctatgaaaaaattattattcgcaattcctttatgtctcagctacgaaaccgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaatggtggtagcggtggttctatcaaaattgccacgcgtaaatatttaggcaaacagaatgtttatgatatcggtgtcgagcgcgatcataatttcgcgctgaaaaacggctttatcgccagcaattgttttaatgttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaaacttaattaacggcactcctcagccaagtcaaaagcctccgaccggaggcttttgactacatgcccatggcgtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgaaggccaaataggccgt

SEQ ID NO: 94 - DNA, 10810 bp, synthetic construct
  misc_feature 1..10810: note = synthetic
  source 1..10810: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaaggtactctctaatcctgacctgttggagtttgcttccggtctggttcgctttgaggctcgaattaaaacgagatatttgaagtctttcgggcttcctcttaatctttttgatgcaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatctctcacctaccaaacaatgcccccctgcaaaaaataaattcatataaaaaacatacagataaccatctgcggtgataaattatctctggcggtgttgacataaataccactggcggttatactgagcacgggtaccgccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtatataccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctccagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 95 - DNA, 7126 bp, synthetic construct
  misc_feature 1..7126: note = synthetic
  source 1..7126: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaggatactctctaatcctgacctgttggagtttgcttccggtctggttcgctttgaagctcgaattaaaacgagatatttgaagtctttcgggcttcctcttaatctttttgatacaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtatataccttaaagaagcgtactttgtagtgctcacacagattgtctgatagaaagtgaaaagcaaggcgtttacgcgttgggagtgaggcagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 96 - DNA, 10810 bp, synthetic construct
  misc_feature 1..10810: note = synthetic
  source 1..10810: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaaggtactctctaatcctgacctgttggagtttgcttccggtctggttcgctttgaggctcgaattaaaacgagatatttgaagtctttcgggcttcctcttaatctttttgatgcaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatctctcacctaccaaacaatgcccccctgcaaaaaataaattcatataaaaaacatacagataaccatctgcggtgataaattatctctggcggtgttgacataaataccactggcggttatactgagcacgggtaccgccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctccagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 97 - DNA, 7126 bp, synthetic construct; identical to SEQ ID NO: 95
  misc_feature 1..7126: note = synthetic
  source 1..7126: mol_type = other DNA

SEQ ID NO: 98 - DNA, 10811 bp, synthetic construct; identical to SEQ ID NO: 85
  misc_feature 1..10811: note = synthetic
  source 1..10811: mol_type = other DNA

SEQ ID NO: 99 - DNA, 7126 bp, synthetic construct
  misc_feature 1..7126: note = synthetic
  misc_feature 3683..3688: note = n is a, c, g, or t
  source 1..7126: mol_type = other DNA
atgattgacatgctagttttacggttaccgttcatcgattctcttgtttgctccagactctcaggcaatgacctgatagcctttgtagatctctcaaaaatagctaccctctccggcatgaatttatcagctagaacggttgaatatcatgttgatggtgatttgactgtctccggcctttcccacccttttgaatctttacctacacattactcaggcattgcatttaaaatatatgagggttctaaaaatttttatccttgcgttgaaataaaggcttctcccgcaaaagtattacaggggcataatgtttttggtacaaccgatttaggtttatgctctgaggctttattgcttaattttgctaattctttgccttgcctgtatgatttattggatgttaacgctactactattagtagaattgatgccgccttttcagcccgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggaacaattaaggatactctctaatcctgacctgttggagtttgcttccggtctggttcgctttgaagctcgaattaaaacgagatatttgaagtctttcgggcttcctcttaatctttttgatacaatccgctttgcttctgactataatagtcagggtaaagacctgatttttgatttatggtcattctcgttttctgaactgtttaaagcatttgaggggaattcaatgaatatttatgacgattccgcagtattggccgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagttttgttacgttgatttgggtaatgaatatccggttcttgtcaagattactcttgatgaaggtcagccagcctatgcgccgggtctgtacaccgttcatctgtcctctttcaaagttggtcagttcggttcccttatgattgaccgtctgcgcctcgttccggctaagtaacatggagcaggtcgcggatttcgacataatttatcaggcgatgatacaaatctccgttgtactctgtttcgcgcttggtataatagctgggggtcaaagatgagtgttttagtgtattctttcgcctctttcgttttaggttggtgcctccgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagttgataaactgatacaattaaaggctccttttggagcctttttttttgatgcggccgcgatgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcannnnnntaccttaaagaagcgtactttgtagtgctcacacagattgtctgatagaaagtgaaaagcaaggcgtttacgcgttgggagtgaggcagaaggagattttcaacatgctccctcaatcggttgaatgtcgcccttttgtctttggcgctggtaaaccatatgaattttctattgattgtgacaaaataaacttattccgtggtgtctttgcgtttcttttatatgttgccacctttatgtatgtatcttctacgtttgctaacatactgcgtaataaggagtcttaatcatgccagttcttttgggtattccgttattattgcgtttcctcggtttccttctggtaactttgttcggctatctgcttacttttctcaaaaagggcttcggtaagatagctattgctatttcattgtttcttgctcttattattgggcttaactcaattcttgtgggttatctctctgatattagtgctcaattaccctctgactttgttcagggtgttcagttaattctcccgtctaatgcgcttccctgtttttatgttattctctctgtaaagactgctattttcatttttgacgttaaacaaaaaatcgtttcttatttggattgggacaaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggattgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatacgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcatcgtcttaatctaagctatcgctatgttttcaaggattctaagggaaaattaattaatagcgacgatttacagaagcaaggttattcacttacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttataacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcacctgataatcaggaatatgatgataattccgctccttctggtggtttctttgtcccgcaaaatgataatgttactcaaacttttaaaattaataacgttcgggcaaaggatttaatacgagttgtcgaattgtttgtaaagtctaatacttctaaatcctcaaatgtattatctattgacggatctaatctattagttgttagtgctcctaaagatattttagataacctttctcaattcctttcaactgttgatttgccaactgaccaggtattgattgagggtttgatatttgaggttcagcaaggtgatgctttagatttttcatttgctgctggctctcagcgtggcactgttgcaggcggagttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctattggccagaatgtcccttttattactggtcgtgtaactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaaccaaagaagtattgctacaacggttaatttgcgtgatggacagactcttctactcggtggcctcactgattataaaaacacttctcaggattctggcgtaccgttcctgtctaaaatccctttaatcggcctcctgtttagctcccgctctgattctaacgaggagagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagcgctaaatcgggggcccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgaactgatttaacaaaaatttaacgcgaattttaacaaaatattaacgtttacaatttaaatatttgcttatacaatcttcctgtcttgggggcttttcttattatcaaccggggtacat

SEQ ID NO: 100 - DNA, 8356 bp, synthetic construct
  misc_feature 1..8356: note = synthetic
  source 1..8356: mol_type = other DNA
atgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatgttgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgctagcagtgagcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgcccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataaccccccttgtattactgtttatgtaagcagacagttttattgttcatgatgatatatttttatcttgtgcaatgtaacatcagagattttgaaacacaacgtggctttcccccccccccccctgcaggtctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttcttattatcaaccggggtacat

SEQ ID NO: 101 - DNA, 2533 bp, synthetic construct
  misc_feature 1..2533: note = synthetic
  source 1..2533: mol_type = other DNA
tgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgttagccatgagggtttagttcgttaaacatgagagcttagtacgttaaacatgagagcttagtacgtgaaacatgagagcttagtacgttaaacatgagagcttagtacgtgaaacatgagagcttagtacgtactatcaacaggttgaactgctgatcttcagatcagagcggatccgtggccggccagccgctccaccgtcaaaagaatagcccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtgctgaggagacttagggaccctggtttcccgactggaaagcgggcagtgagcgcaacgcaattaatgtgagttagctcactcattaggcaccccaggctttacactttatgcttccggctcgtatgttgtgtggaattgtgagcggataacaatttcacacacctgcaggtgcagtagggagtccacaatggtcagtaaaggagaagaacttttcactggagttgtcccaattcttgttgaattagatggtgatgttaatgggcacaaattttctgtcagtggagagggtgaaggtgatgcaacatacggaaaacttacccttaaactgatttgcactactggaaaactacctgttccatggccaacacttgtcactactctgggctatggtctgcaatgctttgccagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcaccgcagacaaacaaaagaatggaatcaaagccaacttcaaaattagacacaacattgaagatggaggcgttcaactagcagaccattatcaacaaaatactccaattggcgatggccctgtccttttaccagacaaccattacctgtcctaccaatctgccctttcgaaagatcccaacgaaaagagagaccacatggtccttcttgagtttgtaacagctgctgggattacacatggcatggatgaactatacaaataaacttaattaacggcactcctcagccaagtcaaaagcctccgaccggaggcttttgactacatgcccatggcgtttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccaggccaaataggccgt

SEQ ID NO: 102 - DNA, 1766 bp, synthetic construct
  misc_feature 1..1766: note = synthetic
  source 1..1766: mol_type = other DNA
tttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtatataccttaaagaagcgtactttgtagtgctcacacagattgtctgatagaaagtgaaaagcaaggcgtttacgcgttgggagtgaggc

SEQ ID NO: 103 - DNA, 5310 bp, synthetic construct
  misc_feature 1..5310: note = synthetic
  source 1..5310: mol_type = other DNA
tttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacttgtataccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcc

SEQ ID NO: 104 - DNA, 5310 bp, synthetic construct
  misc_feature 1..5310: note = synthetic
  source 1..5310: mol_type = other DNA
tttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcaatgtattaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcc

SEQ ID NO: 105 - DNA, 7430 bp, synthetic construct
  misc_feature 1..7430: note = synthetic
  source 1..7430: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacttgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 106 - DNA, 7437 bp, synthetic construct
  misc_feature 1..7437: note = synthetic
  source 1..7437: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataacgctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 107 - DNA, 7435 bp, synthetic construct
  misc_feature 1..7435: note = synthetic
  source 1..7435: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggagcttgattccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 108 - DNA, 7410 bp, synthetic construct
  misc_feature 1..7410: note = synthetic
  source 1..7410: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 109 - DNA, 7429 bp, synthetic construct
  misc_feature 1..7429: note = synthetic
  source 1..7429: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagagacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 110 - DNA, 7414 bp, synthetic construct
  misc_feature 1..7414: note = synthetic
  source 1..7414: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacttgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggagcttgattccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 111 - DNA, 7421 bp, synthetic construct
  misc_feature 1..7421: note = synthetic
  source 1..7421: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataacgctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggagcttgattccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 112 - DNA, 7394 bp, synthetic construct
  misc_feature 1..7394: note = synthetic
  source 1..7394: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggagcttgattccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 113 - DNA, 7413 bp, synthetic construct
  misc_feature 1..7413: note = synthetic
  source 1..7413: mol_type = other DNA
gcggccgcgatctctcacctaccaaacaatgcccccctgcaaaaaataaattcatataaaaaacatacagataaccatctgcggtgatccctatcagtgatagagattgacatccctatcagtgatagagatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggagcttgattccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagagacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcatgtggttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaaccttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 114 - DNA, 2646 bp, synthetic construct
  misc_feature 1..2646: note = synthetic
  source 1..2646: mol_type = other DNA
aacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacggctccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcg